SEQUENCE LISTING (29 sequences)

SEQ ID NO: 1 - RNA, 25 nt, synthetic construct
  source 1..25: mol_type = other RNA
tatggaacctgtctgcacactgcac

SEQ ID NO: 2 - RNA, 20 nt, synthetic construct
  source 1..20: mol_type = other RNA
gacactcggagggcttcact

SEQ ID NO: 3 - RNA, 20 nt, synthetic construct
  source 1..20: mol_type = other RNA
ggccgctgcatatcgtcctg

SEQ ID NO: 4 - DNA, 20 bp, synthetic construct
  source 1..20: mol_type = other DNA
ggagagaagaggagccaggt

SEQ ID NO: 5 - DNA, 20 bp, synthetic construct
  source 1..20: mol_type = other DNA
gctgggggacaatgctaata

SEQ ID NO: 6 - DNA, 20 bp, synthetic construct
  source 1..20: mol_type = other DNA
gcctaacacagatgctgcaa

SEQ ID NO: 7 - DNA, 20 bp, synthetic construct
  source 1..20: mol_type = other DNA
tgctcttaaaccgaggctgt

SEQ ID NO: 8 - DNA, 20 bp, synthetic construct
  source 1..20: mol_type = other DNA
taaactggtggcctcggaac

SEQ ID NO: 9 - DNA, 20 bp, synthetic construct
  source 1..20: mol_type = other DNA
atccccatgcctgggatttg

SEQ ID NO: 10 - DNA, 20 bp, synthetic construct
  source 1..20: mol_type = other DNA
gtgaagtaccctgacctgcc

SEQ ID NO: 11 - DNA, 20 bp, synthetic construct
  source 1..20: mol_type = other DNA
agtcatgcattccttggcga

SEQ ID NO: 12 - DNA, 20 bp, synthetic construct
  source 1..20: mol_type = other DNA
gctgaaggaagctgccctat

SEQ ID NO: 13 - DNA, 20 bp, synthetic construct
  source 1..20: mol_type = other DNA
ttggtctgtaggagcctggt

SEQ ID NO: 14 - DNA, 19 bp, synthetic construct
  source 1..19: mol_type = other DNA
atgcacagcatgagtcggg

SEQ ID NO: 15 - DNA, 25 bp, synthetic construct
  source 1..25: mol_type = other DNA
ctggtatgcatctcaacatcaaaca

SEQ ID NO: 16 - DNA, 20 bp, synthetic construct
  source 1..20: mol_type = other DNA
tgtgcaacaaagagcaggtg

SEQ ID NO: 17 - DNA, 21 bp, synthetic construct
  source 1..21: mol_type = other DNA
tccagtatgatggcgcttgag

SEQ ID NO: 18 - DNA, 20 bp, synthetic construct
  source 1..20: mol_type = other DNA
aagcaccagaaattgcgacg

SEQ ID NO: 19 - DNA, 20 bp, synthetic construct
  source 1..20: mol_type = other DNA
acgagatgggagtgctggta

SEQ ID NO: 20 - DNA, 20 bp, synthetic construct
  source 1..20: mol_type = other DNA
caatctgctcatgccctacc

SEQ ID NO: 21 - DNA, 20 bp, synthetic construct
  source 1..20: mol_type = other DNA
cttgcttctgcgtgatgtcc

SEQ ID NO: 22 - DNA, 18 bp, synthetic construct
  source 1..18: mol_type = other DNA
ttcattgtgggagcagac

SEQ ID NO: 23 - DNA, 18 bp, synthetic construct
  source 1..18: mol_type = other DNA
cagcagtttctccagagc

SEQ ID NO: 24 - DNA, 19 bp, synthetic construct
  source 1..19: mol_type = other DNA
gcagctatcaaccagatcc

SEQ ID NO: 25 - DNA, 20 bp, synthetic construct
  source 1..20: mol_type = other DNA
gatgtaggcagctgtcattc

SEQ ID NO: 26 - DNA, 21 bp, synthetic construct
  source 1..21: mol_type = other DNA
cagaggtggtaccttgccaaa

SEQ ID NO: 27 - DNA, 18 bp, synthetic construct
  source 1..18: mol_type = other DNA
gccgacagtgcaagaccc

SEQ ID NO: 28 - DNA, 18 bp, synthetic construct
  source 1..18: mol_type = other DNA
aactacgcagcgcctccc

SEQ ID NO: 29 - DNA, 23 bp, synthetic construct
  source 1..23: mol_type = other DNA
attttcggttgttgctgatctgt

(19) United States
(12) Patent Application Publication
(10) Pub. No.: US 2024/0139149 A1
(43) Pub. Date: May 2, 2024

(54) THERAPEUTIC USES OF UROLITHIN DERIVATIVES

(71) Applicant: Vandria SA, Lausanne (CH)

(72) Inventors: Penelope Andreux, Eclepens (CH); Klaus Andreas Dugi, Lausanne (CH); Christopher L. Rinsch, Lausanne (CH); Florian Greten, Bad Homburg (DE); Dominic Denk, Frankfurt (DE)

(21) Appl. No.: 18/226,621

(22) Filed: Jul. 26, 2023

Related U.S. Application Data
(60) Provisional application No. 63/412,078, filed on Sep. 30, 2022.
(60) Provisional application No. 63/392,606, filed on Jul. 27, 2022.

(51) Int. Cl. A61K 31/366; A61K 31/352; A61K 31/365; A61K 31/38; A61K 31/382; A61K 31/397; A61K 31/436; A61K 31/453; A61K 31/473; A61K 31/496; A61K 31/5377; A61K 31/5415; A61K 31/55; A61P 9/04; A61P 21/00; A61P 35/00

(52) U.S. Cl. CPC A61K 31/366; A61K 31/352; A61K 31/365; A61K 31/38; A61K 31/382; A61K 31/397; A61K 31/436; A61K 31/453; A61K 31/473; A61K 31/496; A61K 31/5377; A61K 31/5415; A61K 31/55; A61P 9/04; A61P 21/00; A61P 35/00

(57) ABSTRACT

Disclosed are methods for treating a neuromuscular disorder, muscle disorder, heart disease, pulmonary fibrosis, liver disease, inflammatory bowel disease, or cancer. Also disclosed is a method of enhancing cancer immunotherapy.

### RELATED APPLICATIONS

This application claims the benefit of priority to U.S. Provisional Patent Application Nos. 63/412,078, filed Sep. 30, 2022; and 63/392,606, filed Jul. 27, 2022.

### REFERENCE TO A SEQUENCE LISTING XML

This application contains a Sequence Listing which has been submitted electronically in XML format. The Sequence Listing XML is incorporated herein by reference. Said XML file, created on Oct. 2, 2023, is named AZX-02601_SL.xml and is 26,536 bytes in size.

### BACKGROUND

Urolithins have potent effects on the improvement of a number of health conditions, and they have been shown to be highly biologically active in vitro and in vivo. Urolithins have been proposed as treatments of a variety of conditions including conditions related to inadequate mitochondrial activity, including obesity, memory decline, reduced metabolic rate, metabolic syndrome, diabetes mellitus, cardiovascular disease, hyperlipidemia, neurodegenerative diseases, cognitive disorder, mood disorder, stress, anxiety disorder, fatty liver disease and for improving liver function and weight management. In particular, urolithins have been shown to have beneficial effects in the enhancement of muscle function.

### SUMMARY

One aspect of the invention provides methods useful for treating a neuromuscular disorder (e.g. Charcot-Marie-Tooth disease), muscle disorder (e.g. hereditary inclusion body myositis, oculopharyngeal muscular dystrophy, inclusion body myopathy, Paget's disease of bone, frontotemporal gementia, or Duchenne muscular disorder), heart disease (e.g. heart failure), pulmonary fibrosis (e.g. idiopathic pulmonary fibrosis), liver disease (e.g. non-alcoholic steatohepatitis), inflammatory bowel disease (e.g. ulcerative colitis or Crohn's disease), cancer (e.g. bladder cancer, breast cancer, cervical cancer, colorectal cancer, esophageal cancer, head and neck cancer, kidney cancer, leukemia, liver cancer, lung cancer, lymphoma, melanoma, prostate cancer, or skin cancer), or cognitive impairment.

Accordingly, provided herein is a method of treating a a neuromuscular disorder (e.g. Charcot-Marie-Tooth disease), muscle disorder (e.g. hereditary inclusion body myositis, oculopharyngeal muscular dystrophy, inclusion body myopathy, Paget's disease of bone, frontotemporal gementia, or Duchenne muscular disorder), heart disease (e.g. heart failure), pulmonary fibrosis (e.g. idiopathic pulmonary fibrosis), liver disease (e.g. non-alcoholic steatohepatitis), inflammatory bowel disease (e.g. ulcerative colitis or Crohn's disease), cancer (e.g. bladder cancer, breast cancer, cervical cancer, colorectal cancer, esophageal cancer, head and neck cancer, kidney cancer, leukemia, liver cancer, lung cancer, lymphoma, melanoma, prostate cancer, or skin cancer) or cognitive impairment in a subject in need thereof, comprising administering to the subject an effective amount of a compound of Formula (Ia), Formula (Ic), Formula (Id), Formula (Ie), Formula (If), Formula (Ih), Formula (Ij), or Formula (Ik).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1A: Schematic representation of AOM model and treatment regimen. FIG. 1B: Tumor incidence of AOM-induced tumors receiving a UA-containing diet or control diet. Data are mean±SD, n=4/5, *p<0.05 by two-sided t-test. One of two independent experiments shown. FIG. 1C: average lesion size of AOM-induced tumors receiving a UA-containing diet or control diet. Data are mean±SD, n=4/5, *p<0.05 by two-sided t-test. One of two independent experiments shown. FIG. 1D: Representative images of swiss-roll sections from AOM-induced colon tumors. Scale bar=2 mm. FIG. 1E: Representative images of CD3+ T cell staining of colons from AOM treated mice as depicted in (A, D). Scale bar=100 m. FIG. 1F: Relative number of CD45+CD3+ T cells in colons of AOM-treated mice at week 24, Data are mean±SD, n=7/8, **p<0.01 by two-sided t-test. FIG. 1G: Organoid treatment scheme. APTK-organoids were incubated in the presence of UA or DMSO for the indicated time points, prior to flow cytometric analysis. FIG. 1H: Quantification of lysosome formation as assessed by lysotracker MFI via flow cytometry after 24 h incubation at the presence of various UA concentrations. Data are mean±SD, n=5/4/4, *p<0.05, **p<0.01 by one-way ANOVA followed by Tukey's multiple comparison test. Representative results from one of two intendent experiments are shown. FIG. 1I: Mitotracker signal of APTK organoids incubated for 24 h at the presence of UA in vitro (n=4/4/4). FIG. 1J: Antigen presentation via MHC-I molecules after 48 h of treatment (n=5/4/4). Data for FIGS. 1I and IJ are mean±SD, **p<0.01 by one-way ANOVA followed by Tukey's multiple comparison test. Results from one of two independent experiments are shown. FIG. 1K: Experimental setup of oral UA administration in mice with established APTK-s.c. tumors. Treatment diet was initiated eleven days after tumor injection and maintained until the end of the experiment. FIG. 1L: Growth curve of mice subcutaneously transplanted with APTK tumors, receiving either UA-containing or control food. Data are mean±SD, n=7, ***p<0.001 by two-sided t-test. One out of two independent experiments are shown. FIG. 1M: End point tumor weight. FIG. 1N: CD8+ T cell infiltration in APTK-s.c. tumors assessed by flow cytometry, normalizing total number of CD8+ T cells to tumor weight. Data are mean±SD, n=7, *p<0.05 and ***p<0.001 by Mann-Whitney test. FIG. 1O: Size of subcutaneous APTK tumors in Rag1−/− mice receiving UA-containing or control food. Treatment was initiated as depicted in FIG. 1K. Data are mean±SD, n=7, depicting one out of two independent experiments. FIG. 1P: CD8+ T cell depleting or isotype control antibodies were applied every two days starting three days after s.c. injection of APTK organoids in C57BL/6 mice. FIG. 1Q: Effect of CD8+ T cell depletion on size of subcutaneous APTK tumors in C57BL/6 mice receiving UA-containing or control food. Data are mean±SEM, isotype n=5, UA food+α-CD8 n=9, other groups n=10, ****p<0.0001 by one-way ANOVA followed by Tukey's multiple comparison test. Data shown represents pooled data from two experiments with comparable results. FIG. 1R: α-PD-1 or isotype antibodies were injected every three days starting five days after s.c. injection of APTK organoids in C57BL/6 mice. FIG. 1S: Effect of (α-PD-1 treatment on size of subcutaneous APTK tumors in C57BL/6 mice receiving UA-containing or control food. Data are mean±SEM, isotype n=5; control food+α-PD-1 n=9; other groups n=10; *p<0.05, **p<0.01 and ****p<0.0001 by one-way ANOVA followed by Tukey's multiple comparison test. Data shown represents pooled data from two experiments with comparable results.

FIG. 2A: Scheme of CD3+ T cell activation, treatment and analysis. FIG. 2B: Granzyme B expression in αCD3/αCD28 stimulated CD3+ T cells in the presence of UA or DMSO after 48 h. Data are mean±SD, n=4, **p<0.01 and ****p<0.0001 by one-way ANOVA followed by Tukey's multiple comparison test. FIG. 2C: IFNγ expression in αCD3/αCD28 stimulated CD3+ T cells in the presence of UA or DMSO after 48 h. Data are mean±SD, n=4, **p<0.01 and ****p<0.0001 by one-way ANOVA followed by Tukey's multiple comparison test. FIG. 2D: Quantification of CD44-CD62L+Sca1Hi T_{SCM} 24 h, 48 h and 72 h after αCD3/αCD28 stimulation. Data are mean±SD, n=4 from four independent experiments, **p<0.01; p***<0.001 by two-way ANOVA followed by Tukey's multiple comparison test. FIG. 2E: Frequency of CD62L+CD44-CD8+ T cells with low mitochondrial membrane potential (TMRMLo) after 48 h in the presence or absence of UA (25 μM). Data are mean±SD, n=5; ****p<0.0001 by two-sided t-test. FIG. 2F: Frequency of CD95HiCD62L+CD44-CD8+ T cells 48 hours after in αCD3/αCD28 stimulation in the presence or absence of UA (25 μM). Data are mean±SD; n=5; ****p<0,0001 by two-sided t-test. (FIG. 2G) Frequency of T_{SCM} (n=7), (FIG. 2H) TCF1 (n=7), (FIG. 2I) PD1Hi (n=6), (FIG. 2J) Tim3Hi (n=6) and (FIG. 2K) CTLA4Hi (n=6) within CD8+TIL of APTK-induced tumors receiving control or UA-containing diet. Data are mean±SD, *p<0.05; **p<0.01; ***p<0,001 by two-sided t-test. (FIG. 2L) TNFα and (FIG. 2M) IFNγ expression in CD8+TIL of APTK-induced tumors receiving control or UA-containing diet upon re-stimualtion with PMA/ionomycin. Data are mean±SD, n=7 per group, **p<0.01 and ***p<0,001 by two-sided t-test.

FIG. 3A: Experimental setup of adoptive cell transfer into Rag1−/− mice: CD3+ T cells from OT-1 donor mice were stimulated for 48 h with αCD3/αCD28 in the presence of UA (25 μM; TUA) or DMSO (TDMSO) prior to transfer. FIG. 3B: Number of splenic CD8+ T cells in Rag1−/− mice one week after adoptive transfer of either UA-treated or control T cells. Data are mean±SD with n=7 per group, *p<0.05 by two-sided t-test. FIG. 3C: Experimental setup of adoptive cell transfer of OT-1 CD3+ T cells in mice bearing APTK-OVA s.c. tumors. Ex vivo stimulation prior to transfer was performed as depicted in FIG. 3A. FIG. 3D: Growth curve of s.c. APTK-OVA tumors treated as depicted in (C), UA: n=6, DMSO: n=7, Data are mean±SD, ***p<0,001 by two-sided t-test. FIG. 3E: Final tumor weight of s.c. transplanted APTK-OVA tumors in Rag1−/− mice transplanted with TUA or TDMSO. Data are mean±SD, **p<0.01 by two-sided t-test. Analysis of TIL of ACT treated Rag1−/− mice carrying APTK-OVA tumors: (FIG. 3F) Expression of CD44, (FIG. 3G) TCF1 and (FIG. 3H) CD62L in CD8+TIL from APTK-OVA induced tumors that had received UA-treated OT-I T cells (n=6) or DMSO treated OT-I T cells (n=7), Data are mean±SD, *p<0.05 by two-sided t-test, n.s. not significant. FIG. 3I: Frequency of exhausted Tim3HiPD-1Hi CD8+TIL from APTKOVA induced tumors that had received UA-treated OT-I T cells (n=6) or DMSO treated OT-I T cells (n=7). Data are mean±SD, *p<0.05 by two-sided t-test.

FIG. 4A: Scheme of CD3+ T cell activation, treatment and analysis. FIG. 4B: Frequency of CD8+ T cells with low mitochondrial membrane potential (TMRMLo) after six hour stimulation with αCD3/αCD28 in the absence or presence of UA (50 μM). Data are mean±SD, n=4; *p<0.05 by two-sided t-test. Data shown represents one of two independent experiments. FIG. 4C: Quantification of lysosome formation in CD8+ T cells after 6 h stimulation with αCD3/αCD28 in the absence or presence of UA (50 μM). Data are mean±SD, n=4; **p<0.01 by two-sided t-test. Data shown represents one of two independent experiments. FIG. 4D: Frequency of MitoTracker Red staining of CD8+ T cells after 24 h stimulation with αCD3/αCD28 in the absence or presence of UA. Data are mean±SD, n=6 (UA) or n=4 (DMSO), **p<0.01, ***p<0.001, ****p<0.0001 by one-way ANOVA followed by Tukey's multiple comparison test. FIG. 4E: MitoTracker expression in T cell subsets 24 h after stimulation with αCD3/αCD28 in the absence or presence of UA. Data are mean±SD, n=5, **p<0.01, ***p<0.001, ****p<0.0001 by one-way ANOVA followed by Tukey's multiple comparison test. FIG. 4F: qPCR analysis of various autophagy/mitophagy associated genes in T cells 24 h after stimulation with αCD3/αCD28 in the absence or presence of UA (50 μM), Data are ±SEM, n=3; *p<0.05,**p<0.01 by two-sided t-test. FIG. 4G: Immunoblot analysis of indicated proteins in T cells 24 h after stimulation with αCD3/αCD28 in the absence or presence of UA (50 μM). FIG. 4H: Quantification of lysosome formation in Pink1−/− CD8+ T cells after 6 h stimulation with αCD3/αCD28 in the absence or presence of UA (50 μM). Data are mean±SD, n=4, n.s., not significant by two-sided t-test. Data shown represents one of two independent experiments. FIG. 4I: Frequency of MitotrackerHi Pink1−/− CD8+ T cells after 48 h stimulation with αCD3/αCD28 in the absence or presence of UA (50 μM). Data are mean±SD, n=4, n.s. not significant by two-sided t-test. Data shown represents one of two independent experiments. FIG. 4J: Frequency of T_{SCM} in Pink1−/− CD8+ T cells after 48 h stimulation with αCD3/αCD28 in the absence or presence of UA (25 and 50 μM), Data are mean±SD, n=4, ***p<0.001 by two-sided t-test. FIG. 4K: Expression of TCF1 in CD62L+CD44-CD8+ cells from Pink1−/− mice after 48 h stimulation with αCD3/αCD28 in the absence or presence of UA (50 μM), Data are mean±SD, n=4, ***p<0.001 by two-sided t-test. FIG. 4L: Growth curve of Pink1−/− mice s.c. injected with APTK-organoids, fed UA-containing or control diet as depicted in FIG. 1K. Data are mean±SD, n=4/group. (FIG. 4M) Frequency of TCFHi, (FIG. 4N) PDHi, (FIG. 4O) Tim3Hi CD8+TIL in APTK-induced tumors in Pink1−/− mice fed either UA or control diet. Data are mean±SD, n=4/group. FIG. 4P: Expression of IFNγ in CD8+TIL from Pink1−/− restimulated ex vivo with PMA/Ionomycin in the presence of Brefeldin A for three hours. Data are mean±SD, n=4. FIG. 4Q: Expression of TNFα in CD8+TIL from Pink1−/− restimulated ex vivo with PMA/Ionomycin in the presence of Brefeldin A for three hours. Data are mean±SD, n=4.

FIG. 5A: Enhanced volcano plot of RNAseq of T cells stimulated for 48 h with αCD3/αCD28 in the absence or presence of UA (50 μM). A log 2 fold change of 1 and a p-value of p<0.05 were considered significant (red, significantly upregulated in UA treated cells; green, significantly upregulated in DMSO treated cells). FIG. 5B: Heatmap of differentially expressed genes associated with immune checkpoints, effector molecules and genes coding for leukocyte migration. FIG. 5C: Heatmap of differentially expressed genes associated with T cell memory vs effector fate decisions. Data underwent z-score normalization for display (n=3 per group). FIG. 5D: Frequency of TCF1Hi expressing CD8+ T cells after 48 h stimulation with αCD3/αCD28 in the presence of UA (50 μM) or the TCF1 inhibitor ICG001 (10 μM) in comparison to DMSO control. Data are mean±SD, n=4, ***p<0.001; ****p<0.0001 by one-way ANOVA followed by Tukey's multiple comparison test. FIG. 5E: Frequency of T_{SCM} after 48 h stimulation with αCD3/αCD28 in the presence of UA (50 μM) and the TCF1 inhibitor ICG001 (10 μM) in comparison to DMSO control. Data are mean±SD, n=4: DMSO, UA, ICG001, n=3: UA+ICG001, *p<0.05, p***<0,001; n.s. not significant by one-way ANOVA followed by Tukey's multiple comparison test. One of two independent experiments are shown. FIG. 5F: Immunoblot analysis of phospho-β-catenin in T cells stimulated for 6 h with αCD3/αCD28 in the absence or presence of UA (50 μM). FIG. 5G: Immunoblot analysis of fractionated T cells stimulated for 6 h with αCD3/αCD28 in the absence or presence of UA (50 μM); c=cytosolic, m=mitochondrial fraction. Experiment was repeated twice. FIG. 5H: Immunofluorescence of PGAM5 (green) in T cells stimulated for 6 h with αCD3/αCD28 in the absence or presence of UA (50 μM). Cells were stained with MitoTracker Red to visualize mitochondria (m, mitochondrial localization; c, cytoplasmatic localization). Scale bar=5 μm. Frequency of (FIG. 5I) T_{SCM}, (FIG. 5J) CD95+, (FIG. 5K) TCF1 expressing Pgam5−/− CD8+ T cells after 48 h stimulation with αCD3/αCD28 in the absence or presence of UA. Data are mean±SD, n=4/group, *p<0.05, n.s. not significant by one-way ANOVA followed by Tukey's multiple comparison test. FIG. 5L: PGC-1α expression in CD8+ T cells after 48 h stimulation with αCD3/αCD28 in the absence or presence of UA (50 μM). Data are mean±SD, n=3, p**<0.01 by two-sided t-test. One of two independent experiments are shown. FIG. 5M: Mitochondrial content of CD8+TIL from APTK-induced tumors from wt mice fed UA or control diet as depicted in FIG. 1K. Data are mean±SD, n=6/group, *p<0.05 by two-sided t-test. PGC-1α expression in CD8+ T cells derived from Pink1−/− KO mice (FIG. 5N) or Pgam5−/− mice (FIG. 5O) after 48 h stimulation with αCD3/αCD28 in the absence or presence of UA (50 μM). Data are mean±SD, n=4. FIG. 5P: Frequency of T_{SCM} after 48 h stimulation with αCD3/αCD28 in the presence of UA (50 μM) or the PGC-1α inhibitor (PGC-1αi, 10 μM). Data are mean±SD, n=4, p**<0.01, ***p<0.001, ****p<0.0001 n.s. not significant by one-way ANOVA followed by Tukey's multiple comparison test.

FIG. 6A: Human PBMC were isolated from healthy donors, T cells were purified and stimulated ex vivo with αCD3/αCD28 in the presence of UA (50 μM) or DMSO control. FIG. 6B: Frequency of human T_{SCM} 48 h after stimulation as shown in FIG. 6A. Data are mean±SD, p****0.0001 by oneway ANOVA followed by Tukey's multiple comparison test. FIG. 6C: Quantification of human TMRMloCD8+ T cells 48 h after stimulation in the presence of UA or DMSO control, Data are mean±SD, p**0.01 by two-sided t-test. FIG. 6D: TCF1 expression in human CD8+ T cells 48 h after stimulation in the presence of UA or DMSO control, Data are mean±SD, p*0.05 by two-sided t-test. In FIG. 6b, representative data of one out of five donors with comparable outcome are shown. FIG. 6E: Experimental layout of CD19-CAR T cell generation and expansion. PBMCS from healthy donors were expanded in the presence of IL-7/IL-15 for three days, followed by VSV-LV transduction. After three days of incubation, killing of Nalm-6 cells is assessed upon 24 h coculture FIG. 6F: Frequency of T_{SCM} within CD19-CAR+CD8+ cells after generation as depicted in FIG. 6E. Data are mean±SD, p****0.0001 by two-sided t-test. Data pooled from three independent experiments. FIG. 6G: Killing potential of CD19-CAR-T cells. Percentage of dead NALM-6 cells upon 24 h co-culture with untransduced or CAR-transduced T cells ±UA/DMSO is shown. Data are mean±SD, n=5/3 (transduced/untransduced); p*0.05, p**<0.01, p****0.0001 by two-way ANOVA followed Sidak's multiple comparison test. FIG. 6H: Experimental layout of CEA-CAR T cell experiments. Following CAR gene transduction (upper panel), CAR T cells were frozen for subsequent experiments after thawing (lower panel). FIG. 6I: Frequency of T_{SCM} within CAR+CD8+ cells specific for CEA. Data are mean±SD, n=4, p****0.0001 by two-sided t-test. Data was pooled from two independent experiments. FIG. 6J: Killing potential of CEA-specific CAR-T cells. Percentage of dead CEA-expressing human CRC organoids upon 72 h co-culture with untransduced or CAR-transduced T cells ±UA/DMSO is shown. Data are mean±SD, n=3; ***p<0.001, p****0.0001, n.s. not significant by two-way ANOVA followed Sidak's multiple comparison test.

FIG. 7A: Representative histograms pertaining to FIGS. 1H-I. Right panel, lysosome formation as assessed by lysotracker MFI via flow cytometry after 24 h. Left panel, Mitotracker signal of APTK organoids incubated for 24 h at the presence of UA in vitro after 24 h incubation at the presence of various UA concentrations. FIG. 7B: Representative gating strategy for identifying naïve T cell (TN; CD44−CD62L+), effector memory cell (TEM; CD44−CD62L−), central memory cell (TCM; CD44+CD62L+) and memory stem cell (Tsc_{M}; CD44+CD62L+Sca1+) subsets within stimulated CD8+ or CD4+ T cells. Quantification of TN (FIG. 7C), TCM (FIG. 7D) and TEM (FIG. 7E) within CD8+ T cells that were stimulated with αCD3/αCD28 for 48 h in the presence of different doses of UA. Data are mean±SD, n=4, p*<0.05, p**<0.01; ***p<0,001; n.s. not significant by twoway ANOVA followed by Tukey's multiple comparison test. Complete subset analysis of CD4+ cells, showing the quantification of TN (FIG. 7F), TCM (FIG. 7G), TEM (FIG. 7H) and T_{SCM} (FIG. 7I) within CD4+ T cells that were stimulated with αCD3/αCD28 for 48 h in the presence of different doses of UA. Data are mean±SD, n=4, p*<0.05, p**<0.01; n.s. not significant by two-way ANOVA followed by Tukey's multiple comparison test.

FIG. 8A: Representative gating strategy for identifying dead cells within murine T cells. FIG. 8B: Analysis of murine T cell death upon stimulation at various concentrations of UA at the indicated time points. Data are mean±SD, n=8-11, ****p<0.0001; n.s. not significant by two-way ANOVA followed by Tukey's multiple comparison test. FIG. 8C: UA restricts T cell proliferation. Representative analysis of T cell proliferation over 72 h in response to UA. FIG. 8D: Quantification of proliferation data obtained from FIG. 8C. Proportion in proliferative generation as assessed via FlowJo Software is shown (Undiv., undivided cells; Gen1, divided once). Data are mean±SD, n=3, p**<0.01; ****p<0.0001; n.s. not significant by two-wayANOVA followed by Tukey's multiple comparison test. One of two independent experiments is shown. FIG. 8E: Representative analysis of cyclin D1 expression after 48 h of αCD3/αCD28 stimulation in the absence of presence of UA (50 μM). Data are mean±SD, n=4, p*<0.05 by two-sided t-test. One of two independent experiments is shown. FIG. 8F: Frequency of T_{SCM} after 48 h of αCD3/αCD28 stimulation in the absence or presence of UA (50 μM). Data are mean±SD, n=5/group, ***p<0.001, ****p<0.0001 by one-way ANOVA followed by Tukey's multiple comparison test. FIG. 8G: Immunoblot analysis of T cells stimulated with αCD3/αCD28 for 6 h in the absence or presence of UA (50 μM). FIG. 8H Representative gating strategy. Quantification of (FIG. 8I) TAM, (FIG. 8J) M-MDSC, (FIG. 8K) PMN-MDSC and (FIG. 8L) DC in APTK-tumors of mice receiving control or UA-containing diet (treatment regimen in FIG. 1K). Data are mean±SD, n=7/group. Statistical analysis was performed by two-sided t-test. FIG. 8M: Frequency of T_{SCM} within CD4+TIL of APTK-induced tumors receiving control or UA-containing diet. Data are mean±SD, n=7, n.s. not significant by two-sided t-test.

FIG. 9A: Scheme of OT-1 CD3+ T cell activation, treatment, and analysis. FIG. 9B: Quantification of OT-1 CD44-CD62L+Sca1Hi T_{SCM} 48 h after αCD3/αCD28 stimulation. Data are mean±SD, n=4 *p<0.05, by two-sided t-test. Data from one out of two experiments are shown. FIG. 9C: Quantification and gating strategy of CD95 expression on OT-1 CD8+ cells 48 h after αCD3/αCD28 stimulation. Data are mean±SD, n=4, *****p<0.0001, by two-sided t-test. Data from one out of two experiments are shown.

FIG. 10A: Ingenuity pathway upstream regulator analysis (IPA) of UA-treated T cells. Assessed from RNAseq data. Plausible upregulation is depicted by z score, significance is shown on the overlying dot spot. Genes with a log 2 fold change of 1 and p<0.05 were initially considered. FIG. 10B: qPCR analysis of selected Wnt target genes 24 h after stimulation. Data are mean±SEM, n=4, *p<0.05, **p<0.01, ***p<0.001 by two-sided t-test. FIG. 10C: Representative histogram for data depicted in FIG. 5K. FIG. 10D: Flow cytometric analysis of lysosome formation in Pgam5−/−CD8+ Tcells after 6 h stimulation with αCD3/αCD28 in the absence or presence of UA (50 μM), Data are mean±SD, *p<0.05, by two-sided t-test. FIG. 10E: Mitotracker Red staining after 24 h stimulation Pgam5−/−CD8+ T-cells with αCD3/αCD28 in the absence or presence of UA (50 μM). Data are mean±SD, n=4, ***p<0.001 by two-sided t-test. FIG. 10F: Immunoblot analysis of cell fractionations of Pink1−/− T cells 6 h stimulation with αCD3/αCD28 in the absence or presence of UA (50 μM). One of two independent experiments is shown (c=cytosolic fraction, m=mitochondrial fraction). FIG. 10G: Representative histograms for data depicted in FIG. 5N. FIG. 10H: Representative histograms for data depicted in FIG. 5M.

FIG. 11A: Representative gating strategy for identification of human T_{SCM} (CD45RA+CCR7HiCD62L+CD95+CD8+). FIG. 11B: Representative flow cytogram, displaying a dose-dependent increase of CD95hiCD62L+ cells within the CD45RA+CCR7hiCD8+ population.

FIG. 12A: Representative gating strategy for assessment of CAR expression and identification of human T_{SCM} in CAR T cell experiments. FIG. 12B: Quantification of CD19 CAR-expression on CD8+ cells after VSV-LV aided gene transduction at the presence of DMSO or UA (25M) for 72 h. Data are mean±SD, n=5, n.s. not significant by two-sided t-test. FIG. 12C: Frequency of exhausted Tim3HiPD-1Hi CD8+CAR+ after activation and CD19 CAR transduction as indicated in FIG. 6E. Data was acquired three days after transduction. Data are mean±SD, n=6, n.s. not significant by two-sided t-test.

FIG. 13A: Graph showing the effect of oral administration of 66 on cardiac muscle in a rat model of heart failure. Ejection fraction, expressed as the difference between Month 2 and day 0 of treatment. FIG. 13B: Graph showing the effect of oral administration of 66 on cardiac muscle in a rat model of heart failure. Fraction shortening, expressed as the difference between Month 2 and day 0 of treatment. Differences in mean delta LV function were assessed between Sham/Vehicle and MI/Vehicle using an unpaired t-test followed by the Welch's correction test. Then, differences between MI/Vehicle group and MI/66 were assessed using an unpaired t-test followed by the Welch's correction test. n per group=10-19. A value of p<0.05 was considered statistically significant. Sham/Vehicle vs MI/Vehicle: **p<0.01; ***p<0.001. MI/Vehicle vs MI/66: #p<0.05.

FIG. 14: Graphs showing the effects of 66, 77, and 77A on the induction of mitophagy in human T lymphocytes. Results are represented as bar graph.

FIG. 15: Graphs showing the effect of 66 on the percentage of T memory stem cells (Tscm). Results are represented as bar graphs.

FIG. 16A: Graph showing the effect of 33 treatment on cortical neurons injured with a chronic application of Aβ1-42. Survival of cortical neurons, as measured with MAP-2 immunostaining. FIG. 16B: Graph showing the effect of 33 treatment on cortical neurons injured with a chronic application of Aβ1-42Neurite network of cortical neurons, as measured with MAP-2 immunostaining. FIG. 16C: Graph showing the effect of 33 treatment on cortical neurons injured with a chronic application of Aβ1-42Microglial activation, as measured with OX-1 immunostaining. Results are expressed as a percentage of control condition (n=4-6). BDNF: brain-derived neurotrophic factor. *p<0.05 after One-way ANOVA followed by Fisher's LSD test

FIG. 17A: Graph showing the effect of 117A treatment on cortical neurons injured with a chronic application of Aβ1-42. Survival of cortical neurons, as measured with MAP-2 immunostaining. FIG. 17B: Graph showing the effect of 117A treatment on cortical neurons injured with a chronic application of Aβ1-42. Neurite network of cortical neurons, as measured with MAP-2 immunostaining. FIG. 17C: Graph showing the effect of 117A treatment on cortical neurons injured with a chronic application of A 1-42. Microglial activation, as measured with OX-1 immunostaining. Results are expressed as a percentage of control condition (n=4-6). BDNF: brain-derived neurotrophic factor. *p<0.05 after One-way ANOVA followed by Fisher's LSD test.

FIG. 18A: The effect of oral administration of 33 on the short-term spatial memory deficit induced by Aβ1-42 intrahippocampal injection (Y-maze test) in aged mice. Graph showing the total distance travelled during Y-maze experiment. FIG. 18B: The effect of oral administration of 33 on the short-term spatial memory deficit induced by Aβ1-42 intrahippocampal injection (Y-maze test) in aged mice. Graph showing the time spent in the new arm (test session). Results are represented as boxplots (n=8-11/group). *p<0.05 after a One-way Anova test followed by fisher's test.

FIG. 19A: The effect of oral administration of 33 on the neurodegeneration and neuroinflammation induced by Aβ1-42 intrahippocampal injection in aged mice. Graphs showing the neuronal survival. Results are represented as boxplots (n=5/group). *p<0.05 after a One-way ANOVA test followed by Fisher's test versus Aβ1-42 group. FIG. 19B: The effect of oral administration of 33 on the neurodegeneration and neuroinflammation induced by Aβ1-42 intrahippocampal injection in aged mice. Graphs showing the hyperphosphorylation of Tau. Results are represented as boxplots (n=5/group). *p<0.05 after a One-way ANOVA test followed by Fisher's test versus Aβ1-42 group. FIG. 19C: The effect of oral administration of 33 on the neurodegeneration and neuroinflammation induced by Aβ1-42 intrahippocampal injection in aged mice. Graphs showing the microglial activation. Results are represented as boxplots (n=5/group). *p<0.05 after a One-way ANOVA test followed by Fisher's test versus Aβ1-42 group.

FIG. 20A: The effect of oral administration of 66 on cardiac muscle in a rat model of heart failure. Ejection fraction, expressed as the difference between Month 2 and day 0 of treatment. FIG. 20B: The effect of oral administration of 66 on cardiac muscle in a rat model of heart failure. Fraction shortening, expressed as the difference between Month 2 and day 0 of treatment. Differences in mean delta LV function were assessed between Sham/Vehicle and MI/Vehicle using an unpaired t-test followed by the Welch's correction test. Then, differences between MI/Vehicle group and MI/66 were assessed using an unpaired t-test followed by the Welch's correction test. n per group=10-19. A value of p<0.05 was considered statistically significant. Sham/Vehicle vs MI/Vehicle: **p<0.01; ***p<0.001. MI/Vehicle vs MI/VNA-052: #p<0.05

FIG. 21: Graph which shows the mitochondrial content of activated mouse T lymphocytes after 72 h of treatment with either DMSO or NCEs at 2 μM. Results are represented as bargraph.

FIG. 22: Graph showing the percentage of mouse T memory stem cells (Tscm) in CD8+T lymphocytes after 72 h of treatment with either DMSO or NCEs at 2 μM. Results are represented as bargraph.

FIG. 23: Graph showing the percentage of human T memory stem cells (Tscm) in CD8+T lymphocytes after 72 h of treatment with either DMSO or 77 at 400 nM. Results are represented as bargraph with mean+/−SEM. ***P<0.001 after unpaired one-sided t-test.

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. “Geometric isomer” means isomers that differ in the orientation of substituent atoms in relationship to a carbon-carbon double bond, to a cycloalkyl ring, or to a bridged bicyclic system.

Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, which contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to an cycloalkyl group as hereinbefore defined substituted with at least one halogen.

“Cycloheteroalkyl” or “heterocycloalkyl” refers to an cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl or heterocycloalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, arylalkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an arylalkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

Methods of Treatment

One aspect of the invention relates to a method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (Ia),

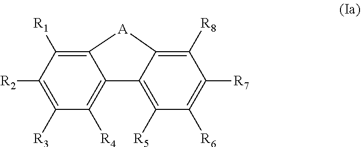

wherein
A is

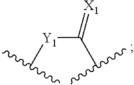

X₁ is selected from O and S;
Y₁ is O;
R₁, R₄, R₅ and R₈ are independently selected from H and halogen;
R₃ and R₆ are independently selected from H, CN, OH, CF₃, halogen, and alkyl; one of R₂ and R₇ is H, OH, or OAc and the other of R₂ and R₇ is halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, C(O)-alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

In some embodiments, the compound provided that if X₁ and Y₁ are each 0, R₂ is OH, and R₁, R₃, R₄, R₅, R₆ and R₈ are each H, then R₇ is not OBn, and if X₁ and Y₁ are each 0, R₇ is OH, and R₁, R₃, R₄, R₅, R₆ and R₈ are each H, then R₂ is not OCH₂C(O)NH₂.

In some embodiments, A is

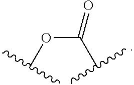 [SMILES: *OC(*)=O]

In some embodiments, R₂ is H. In other embodiments, R₂ is OH. In other embodiments, R₂ is OAc.

In some embodiments, the compound wherein R₂ is selected from haloalkyl, substituted cycloalkyl, alkynyl-R₉, OR₁₀, and C(O)NR₁₁R₁₂; R₉ is selected from OH, substituted cycloalkyl and heterocycloalkyl; R₁₀ is selected from alkyl, substituted cycloalkyl, heterocycloalkyl and alkyl-heterocycloalkyl; and Ru is H and R₁₂ is alkyl-heterocycloalkyl.

In some embodiments, R₇ is H. In other embodiments, R₇ is OH. In other embodiments, R₇ is OAc.

In some embodiments, the compound wherein R₇ is selected from haloalkyl, substituted cycloalkyl, alkynyl-R₉, OR₁₀, and C(O)NR₁₁R₁₂; R₉ is selected from OH, substituted cycloalkyl and heterocycloalkyl; R₁₀ is selected from alkyl, substituted cycloalkyl, heterocycloalkyl and alkyl-heterocycloalkyl; and Ru is H and R₁₂ is alkyl-heterocycloalkyl.

In some embodiments, each occurrence of substituted cycloalkyl is independently substituted with OH, halogen, or hydroxyalkyl.

In some embodiments, R₁, R₃, R₄, R₅, R₆, and R₈ are each H. In other embodiments, one of R₁, R₃, R₄, R₅, R₆, and R₈ is not H. In other embodiments, two of R₁, R₃, R₄, R₅, R₆, and R₈ are not H.

In some embodiments, one of R₁, R₃, R₄, R₅, R₆, and R₈ is alkyl or halogen. In other embodiments, two of R₁, R₃, R₄, R₅, R₆, and R₈ are independently alkyl or halogen.

In some embodiments, the compound of Formula (Ia) is selected from:

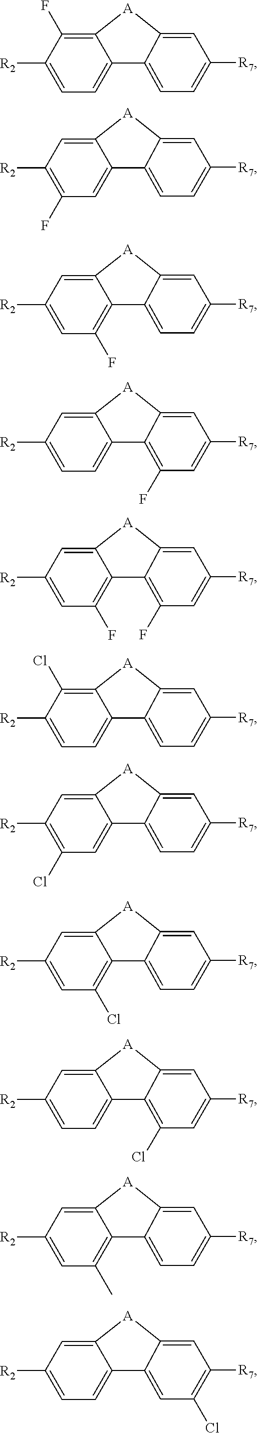

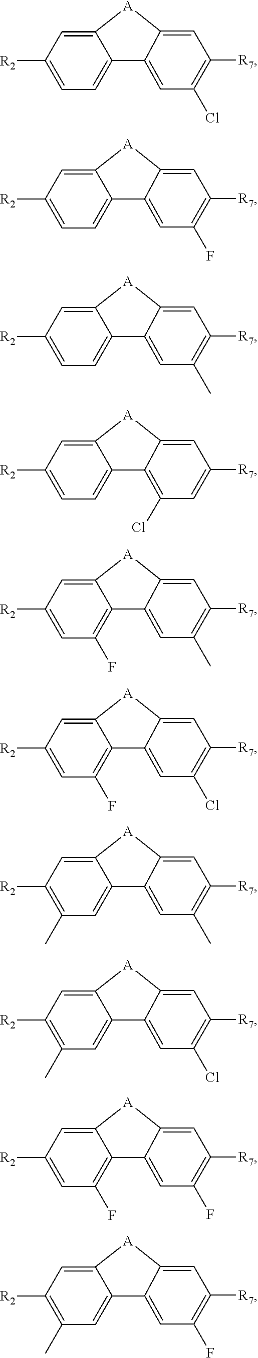

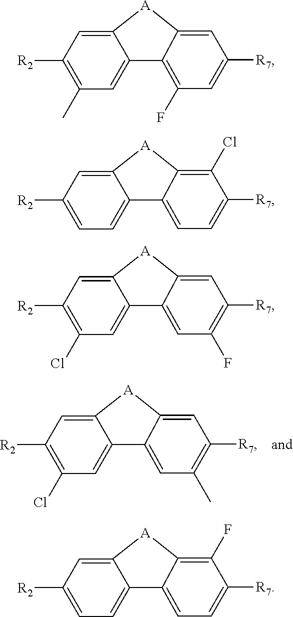

In some embodiments, the compound of Formula (Ia) is selected from:

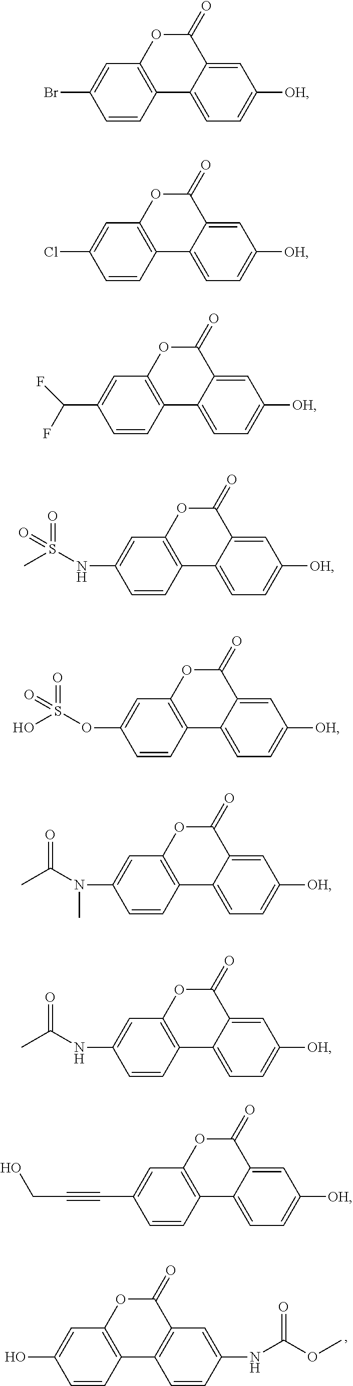

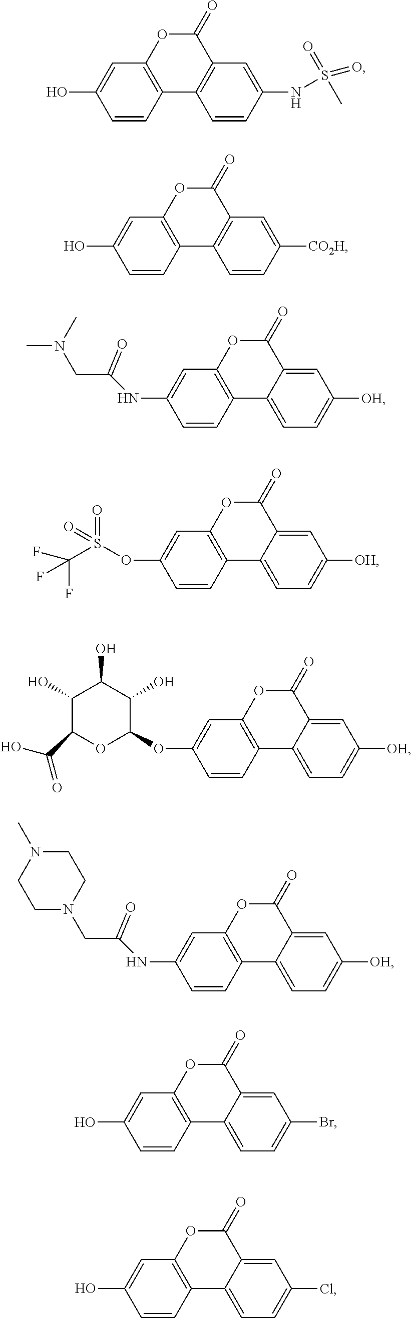

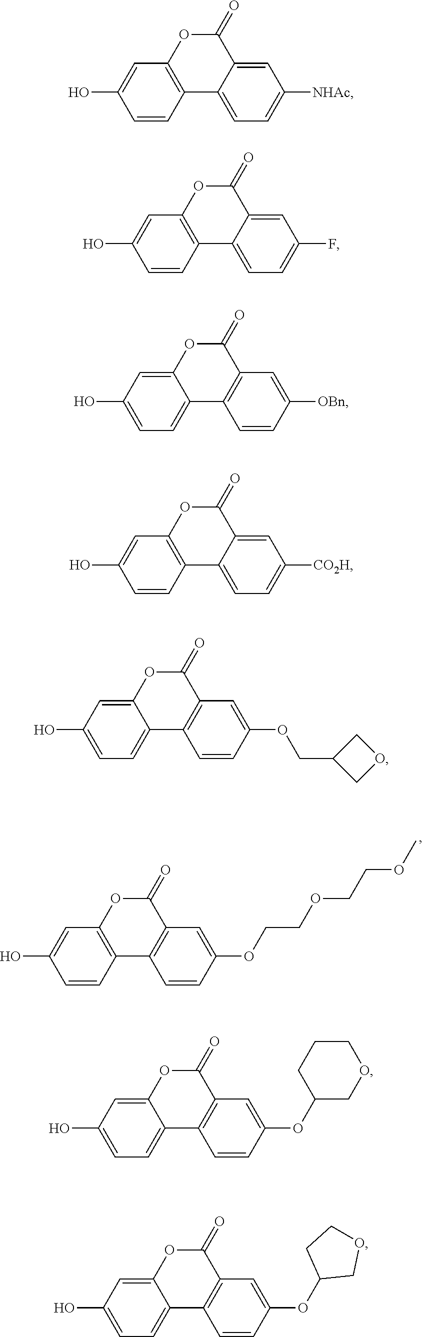

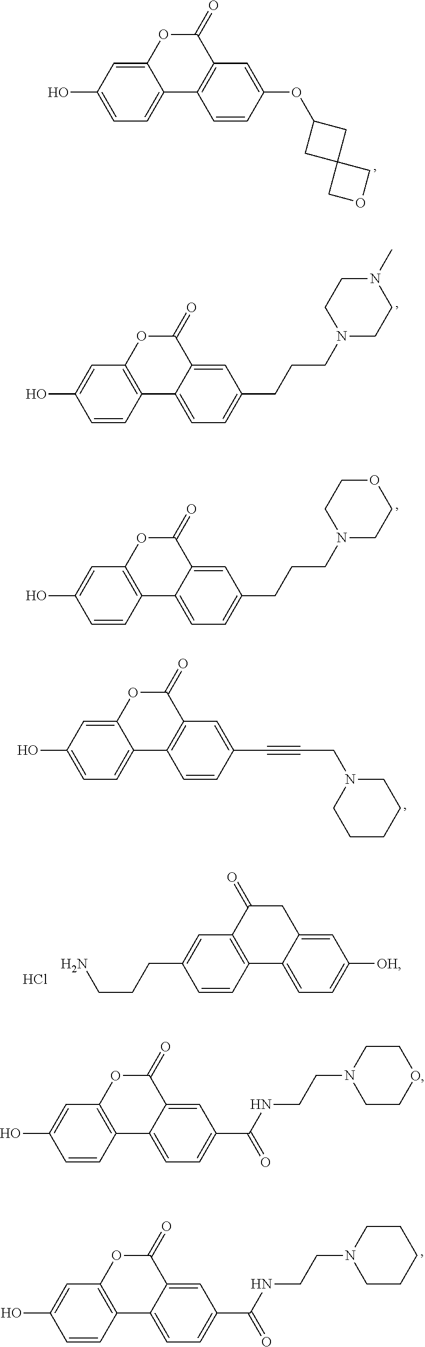

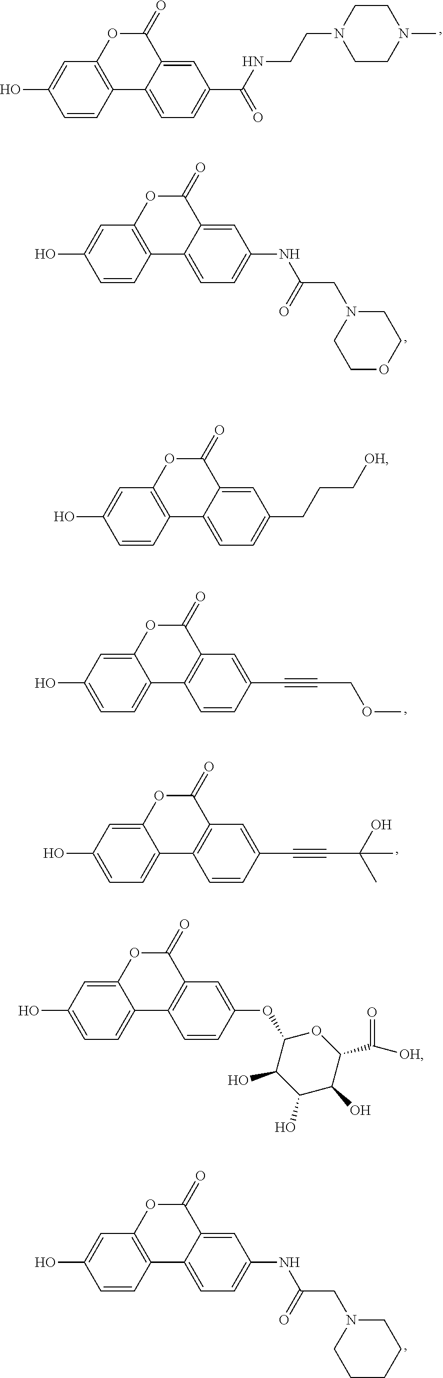

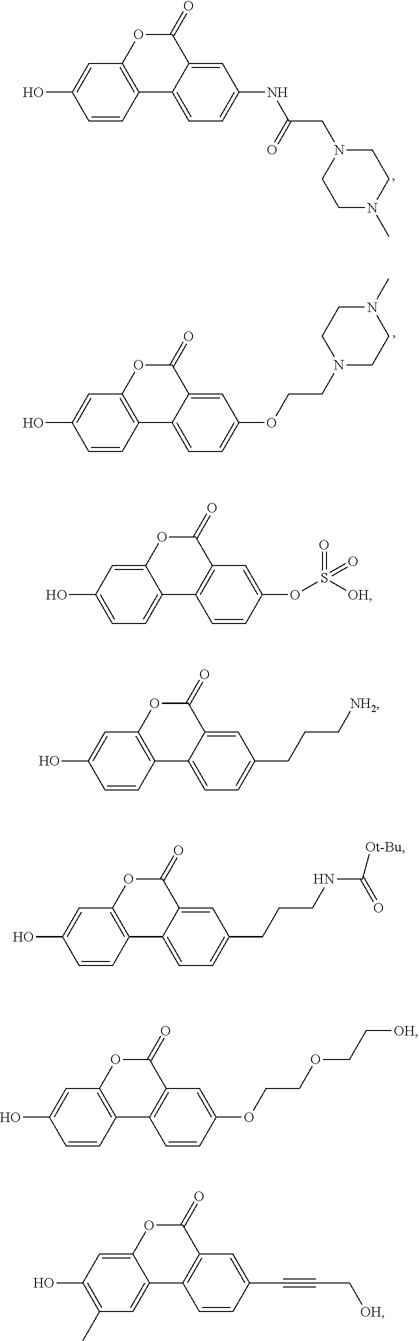

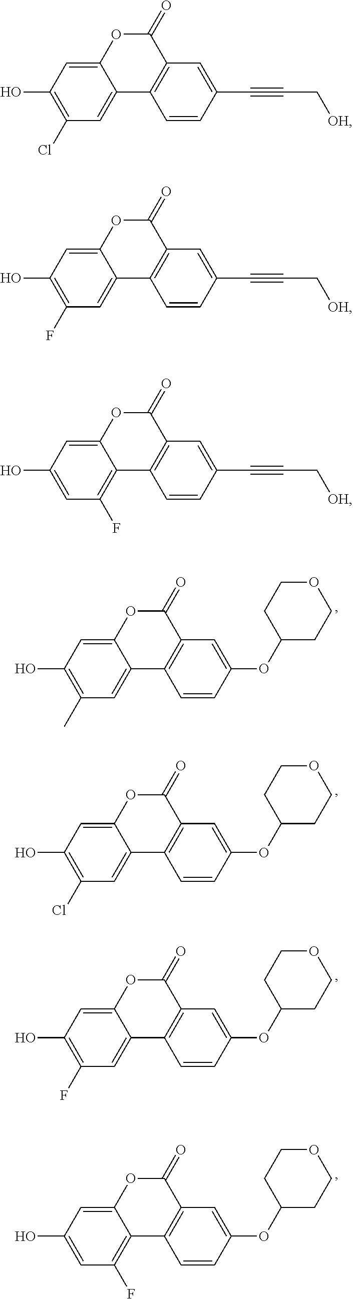

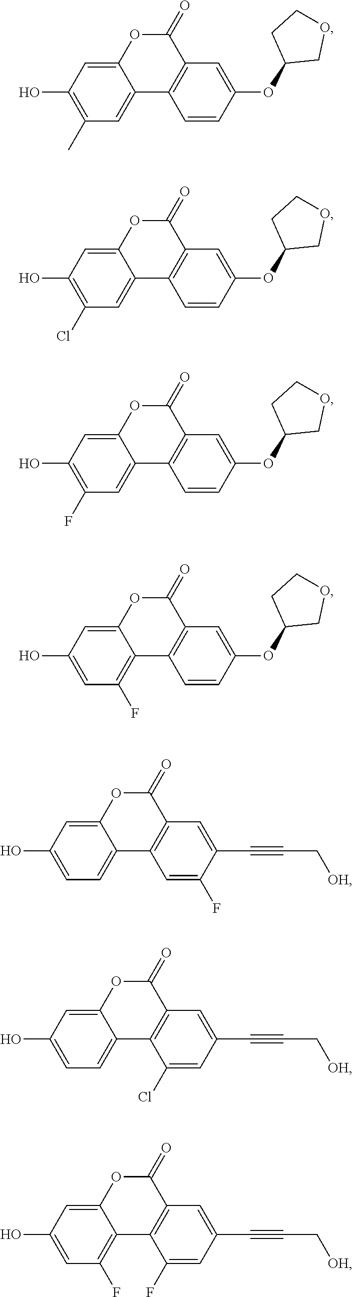

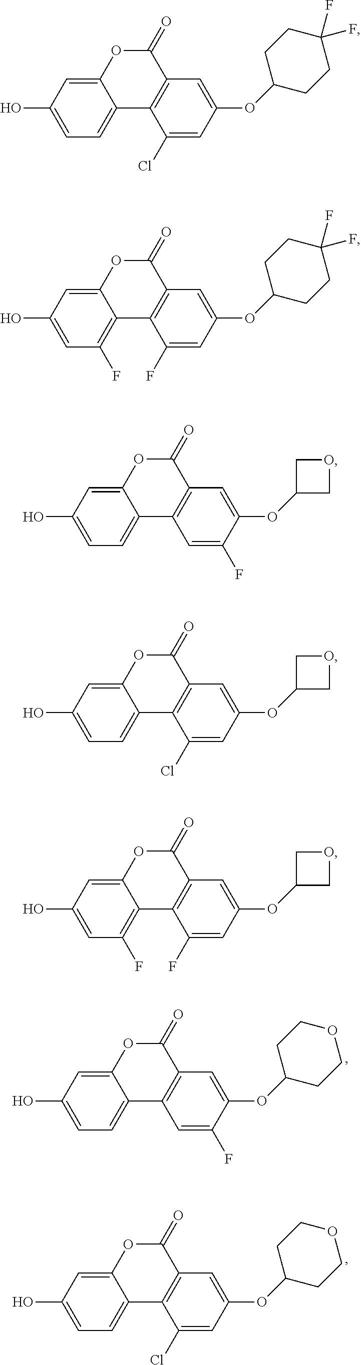

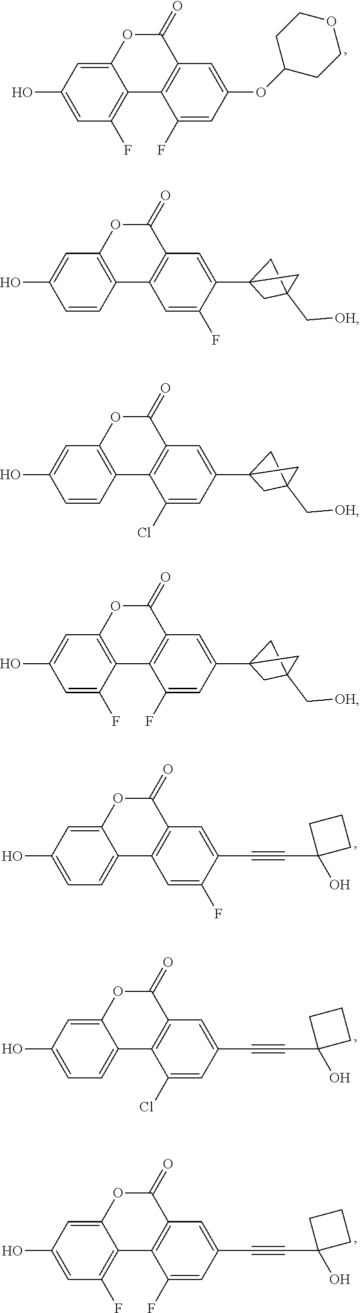

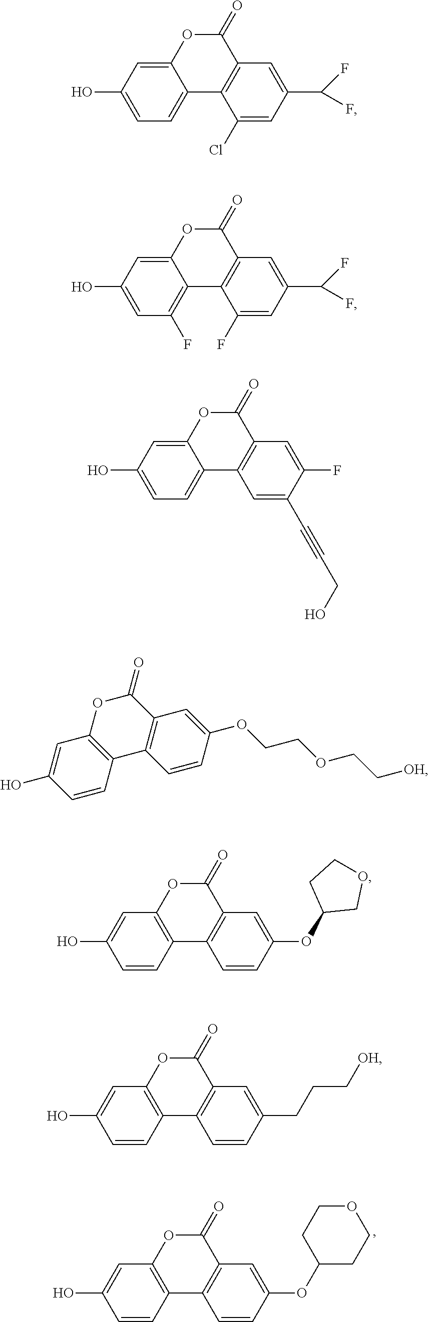

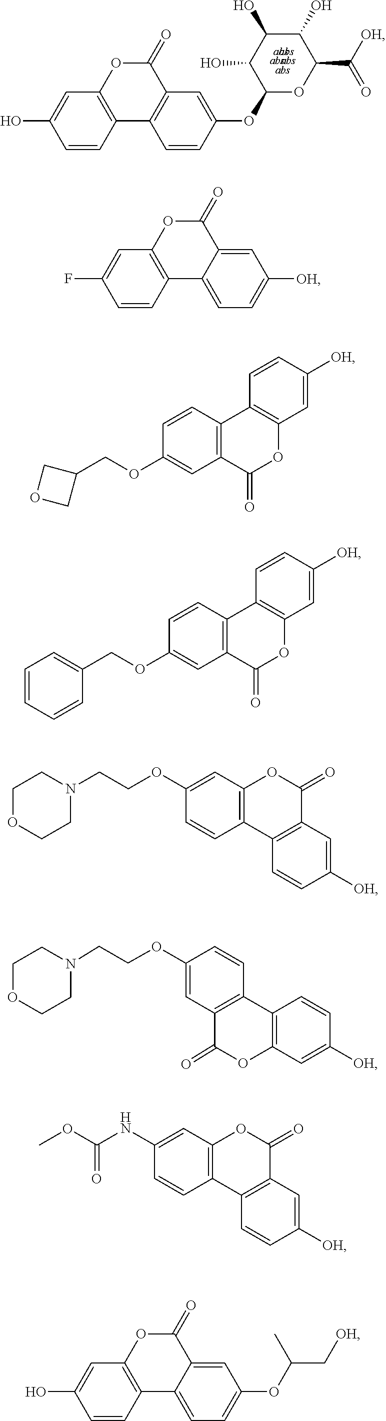

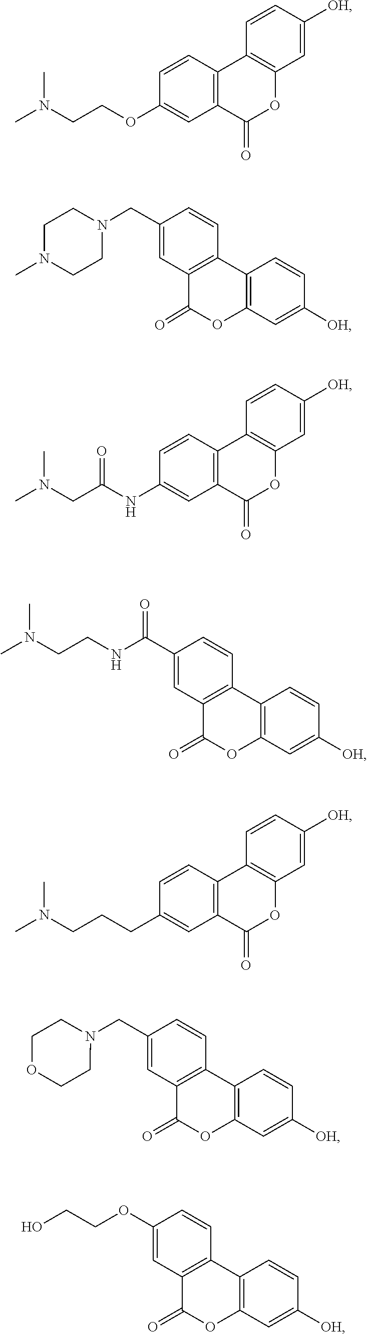

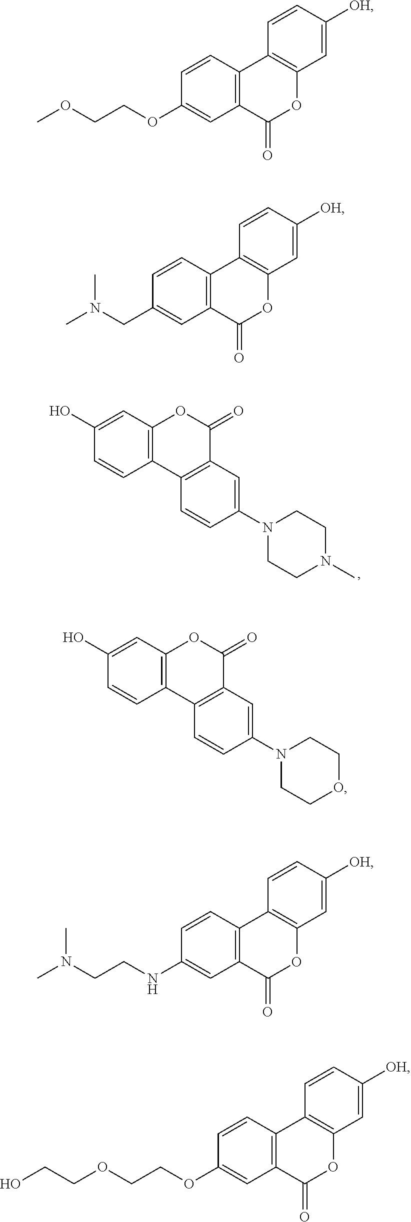

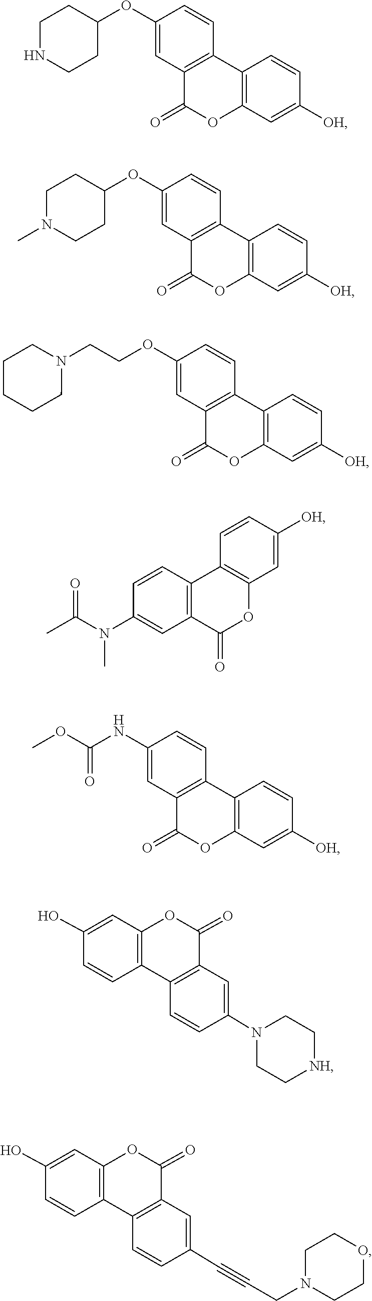

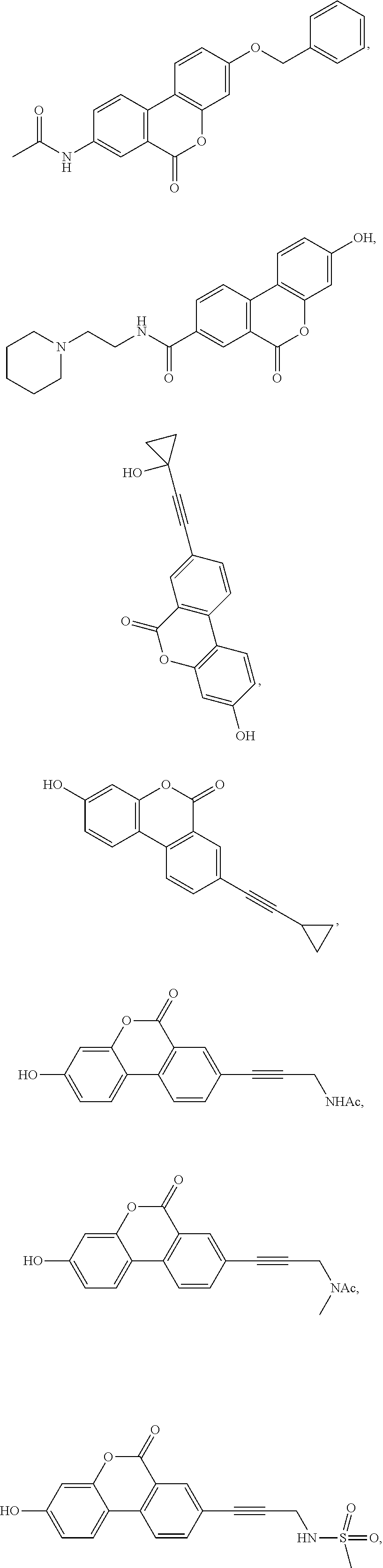

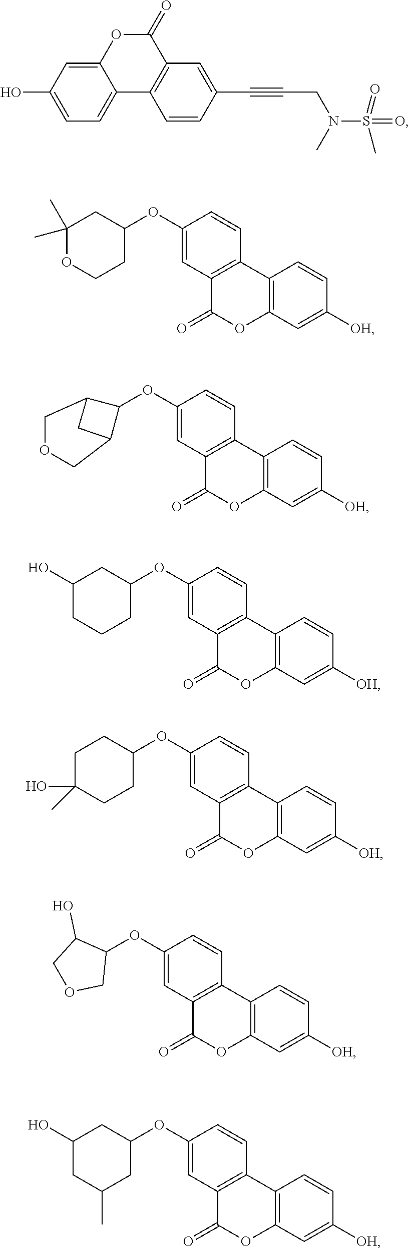

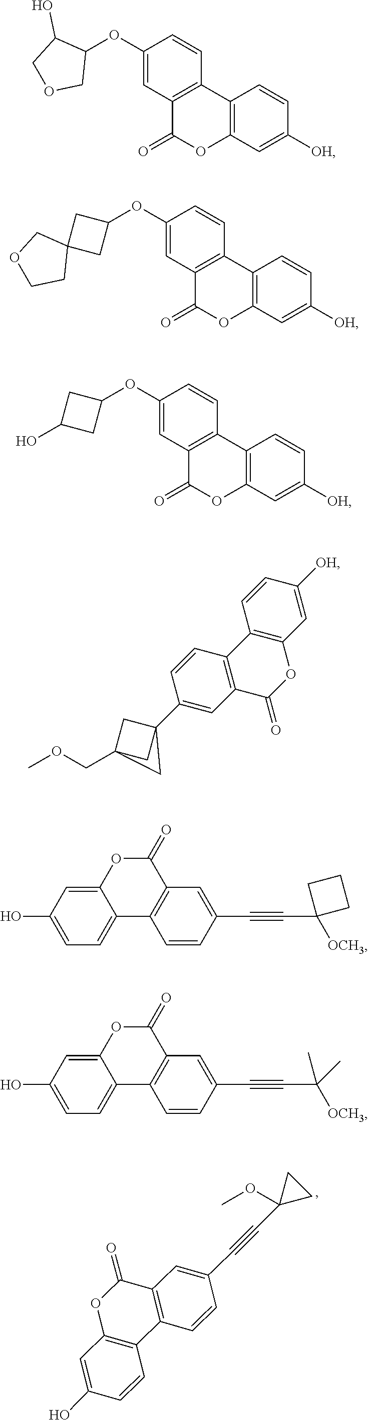

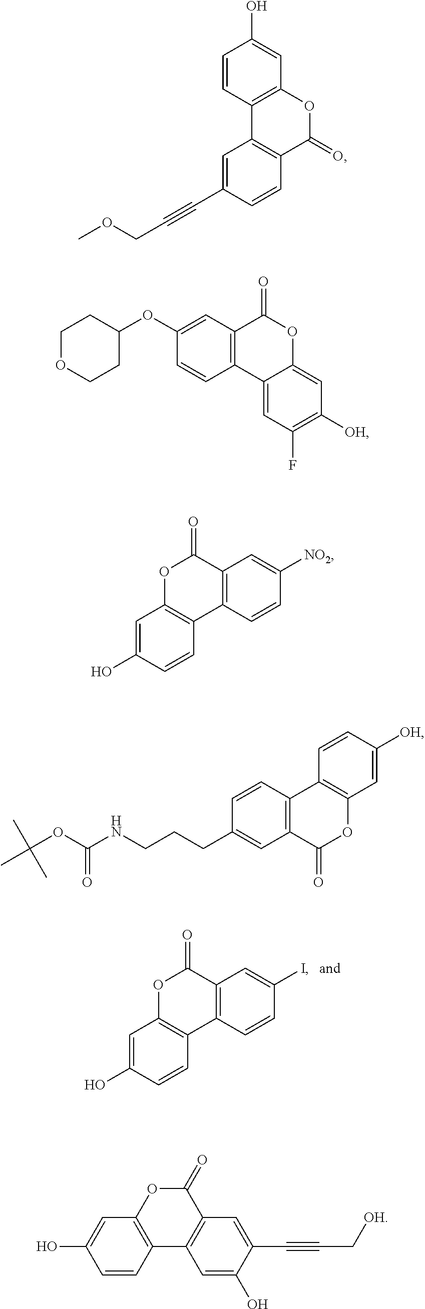

In some embodiments, the compound of Formula (Ia) is selected from:

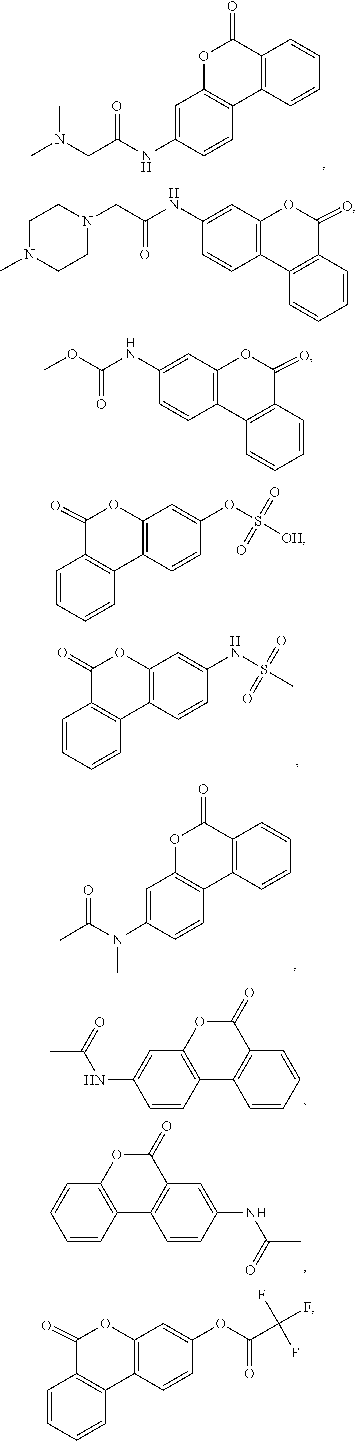

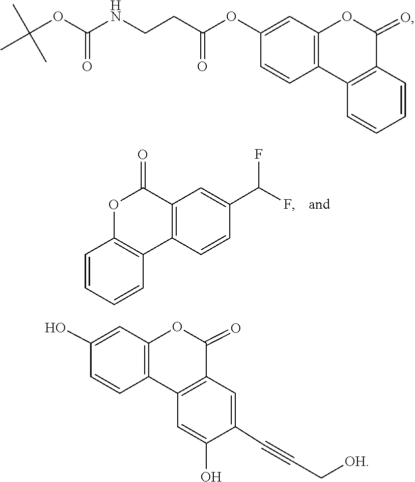

In some embodiments, the compound of Formula (Ia) is selected from:

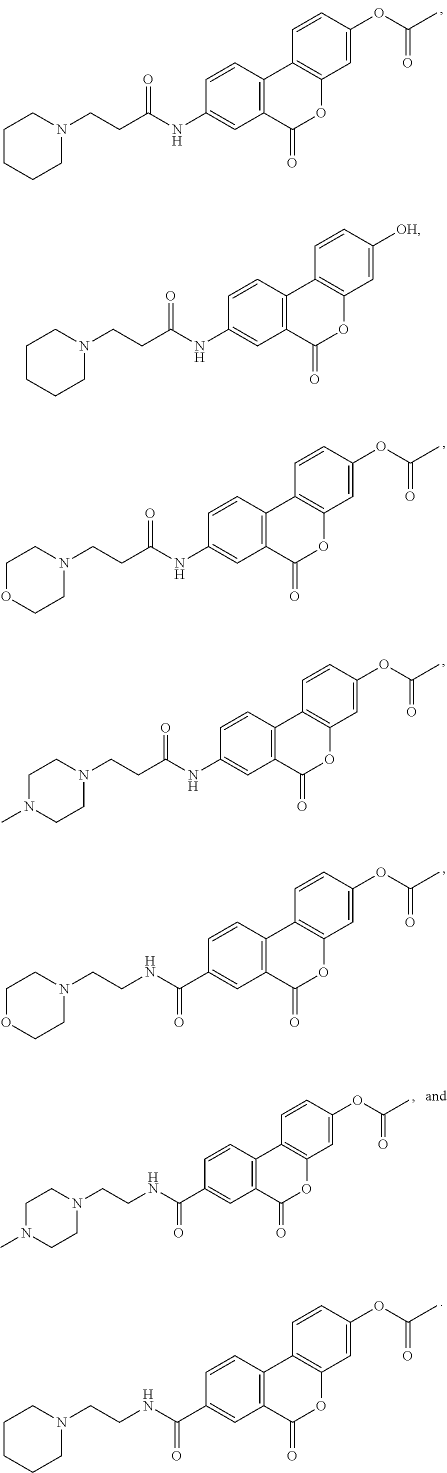

In some embodiments, the compound of Formula (Ia) is selected from:

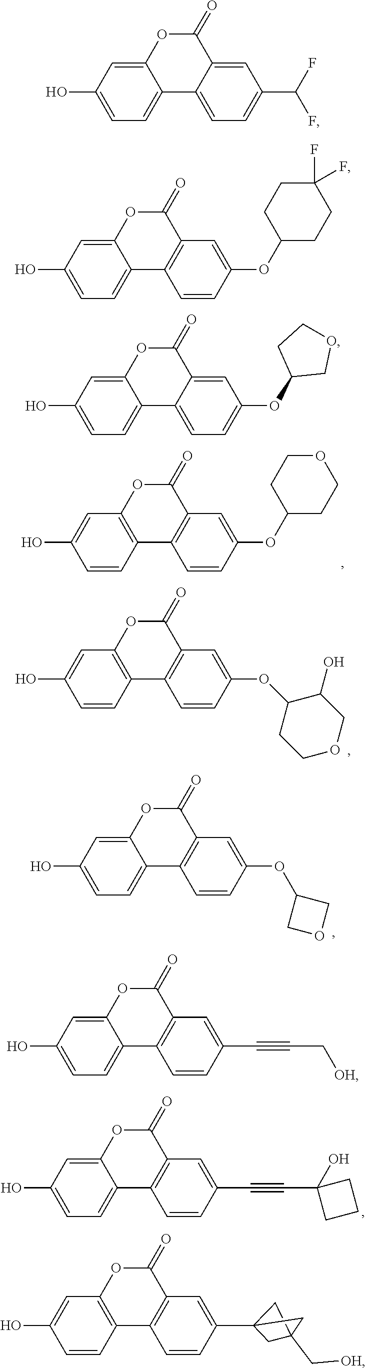

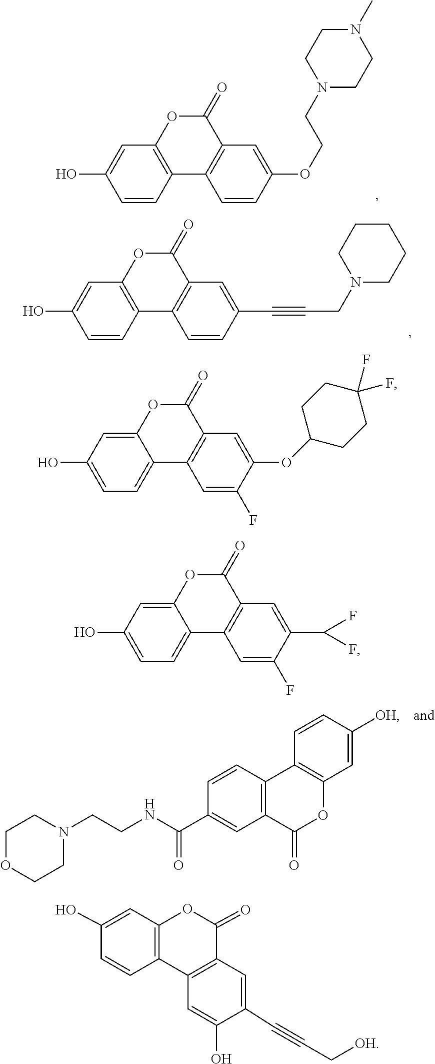

In some embodiments, the compound of Formula (Ia) is selected from:

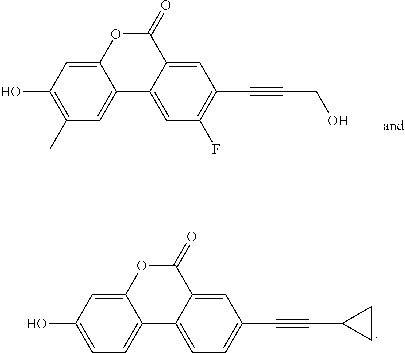 [SMILES: Cc1cc2c(cc1O)oc(=O)c1cc(C#CCO)c(F)cc12 ; O=c1oc2cc(O)ccc2c2ccc(C#CC3CC3)cc12]

Another aspect of the invention relates to method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (Ic),

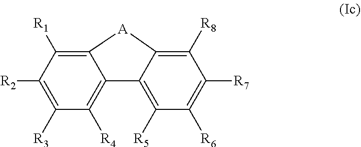

wherein
A is

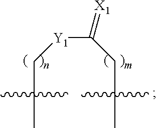

one of n and m is 0; and the other of n and m is 1;
X₁ and Y₁ are each 0;
R₁, R₂, R₃, R₆, R₇, and R₈ are independently selected from H, OH, OCH₃, OAc, NH₂, halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂; R₄ and R₅ are independently selected from H, halogen and alkyl;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of Ru is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

In some embodiments, A is selected from

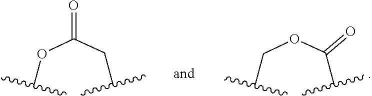 [SMILES: *CC(=O)O* ; *COC(*)=O]

In some embodiments, R₂ and R₇ are each OH. In other embodiments, R₂ and R₇ are each O-alkyl. In other embodiments, R₂ is OH; and R₇ is H or O-alkyl. In other embodiments, R₂ is H or O-alkyl; and R₇ is OH.

In some embodiments, wherein R₁, R₃, R₄, R₅, R₆, and R₈ are each H. In other embodiments, one of R₁, R₃, R₄, R₅, R₆, and R₈ is not H. In other embodiments, two of R₁, R₃, R₄, R₅, R₆, and R₈ are not H. In other embodiments, one of R₁, R₃, R₄, R₅, R₆, and R₈ is alkyl or halogen. In other embodiments, two of R₁, R₃, R₄, R₅, R₆, and R₈ are alkyl or halogen.

In some embodiments, the compound Formula (Ic) is selected from:

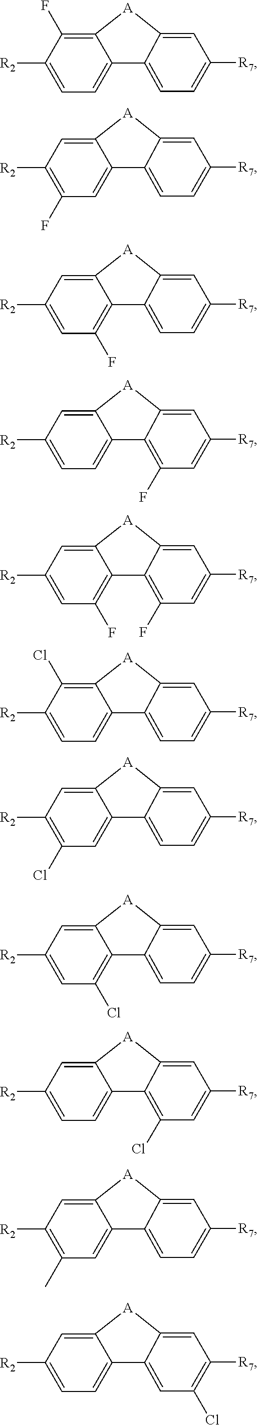

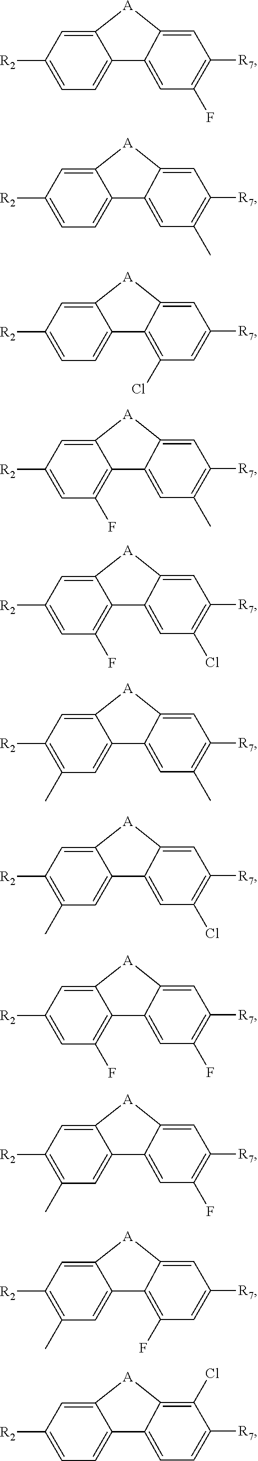

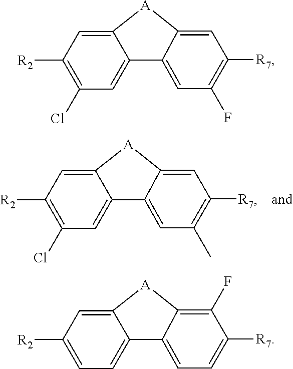

In some embodiments, the compound of Formula (Ic) is selected from:

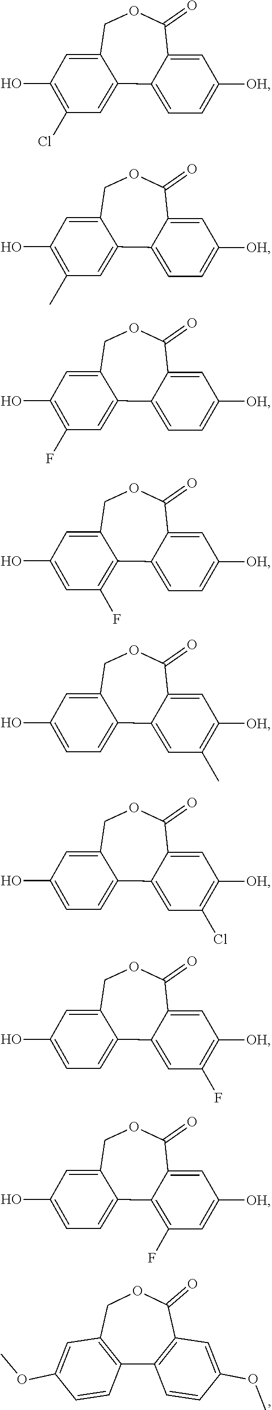

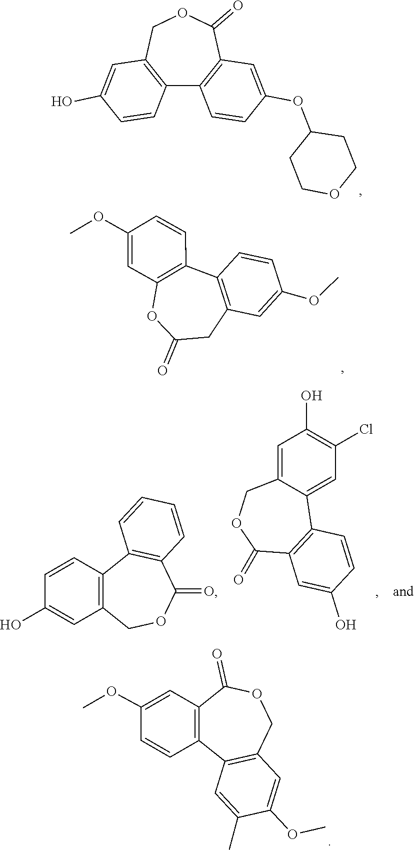

In some embodiments, the compound Formula (Ic) is selected from:

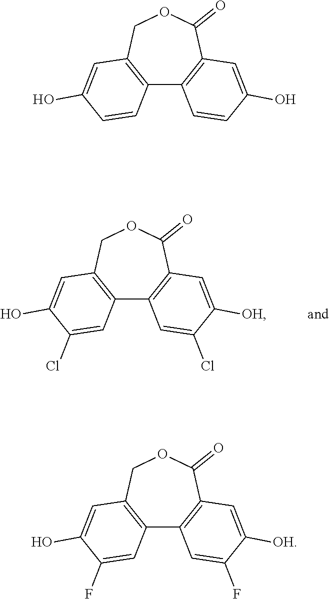 [SMILES: O=C1OCc2cc(O)ccc2-c2ccc(O)cc21 ; O=C1OCc2cc(O)c(Cl)cc2-c2cc(Cl)c(O)cc21 ; "and" ; O=C1OCc2cc(O)c(F)cc2-c2cc(F)c(O)cc21]

Another aspect of the invention relates to a method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (Id),

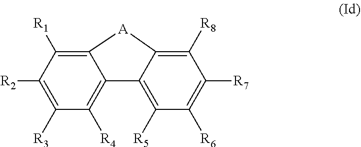

wherein
A is

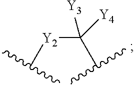

Y₂ is O;
Y₃ and Y₄ are independently selected from H, halogen and alkyl; or together with the carbon to which they are bonded combine to form a cycloalkyl or heterocycloalkyl;
R₁, R₄, R₅, and R₈ are independently selected from H and halogen;
R₂, R₃, R₆, and R₇ are independently selected from H, OH, OCH₃, OAc, NH₂, halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

In some embodiments the compound provided that when Y₂ is 0, R₂ and R₇ are each OH, and R₁, R₃, R₄, R₅, R₆, and R₈ are each H, then Y₃ and Y₄ are not both halogen.

In some embodiments, A is selected from

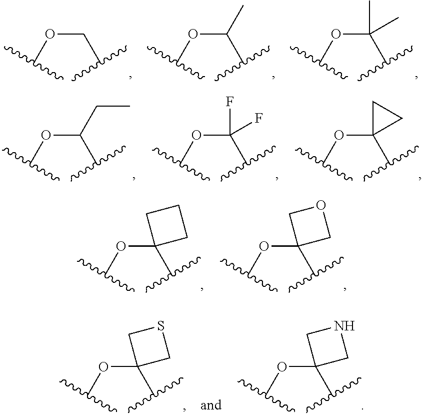

In some embodiments, R₂ and R₇ are each OH. In other embodiments, one of R₂ and R₇ is OH and the other of R₂ and R₇ is O-alkyl. In other embodiments, one of R₁, R₃, R₄, R₅, R₆, and R₈ is alkyl or halogen. In other embodiments, two of R₁, R₃, R₄, R₅, R₆, and R₈ are alkyl or halogen

In some embodiments, the compound Formula (Id) is selected from:

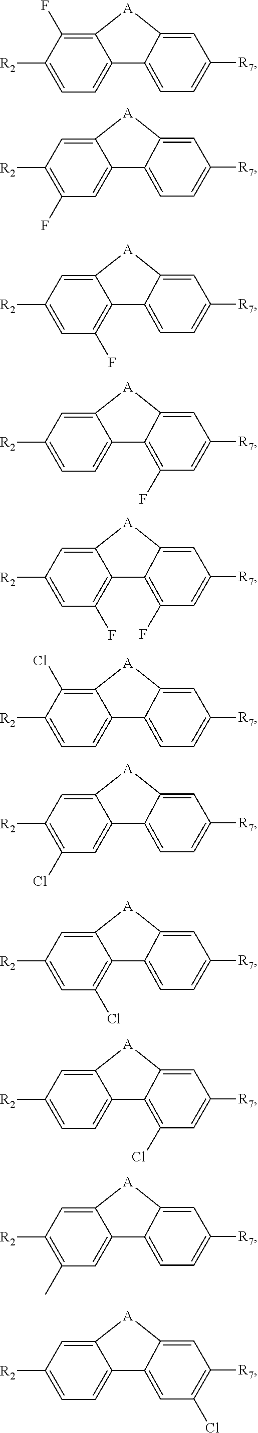

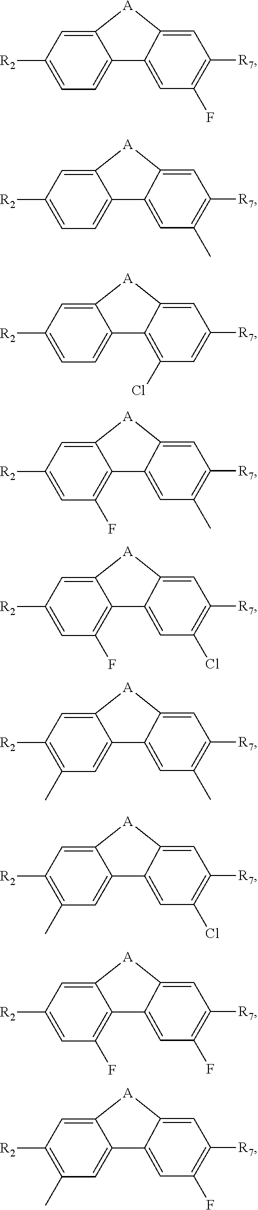

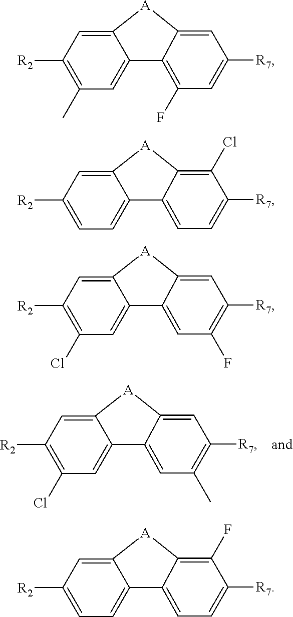

In some embodiments, the compound Formula (Id) is selected from:

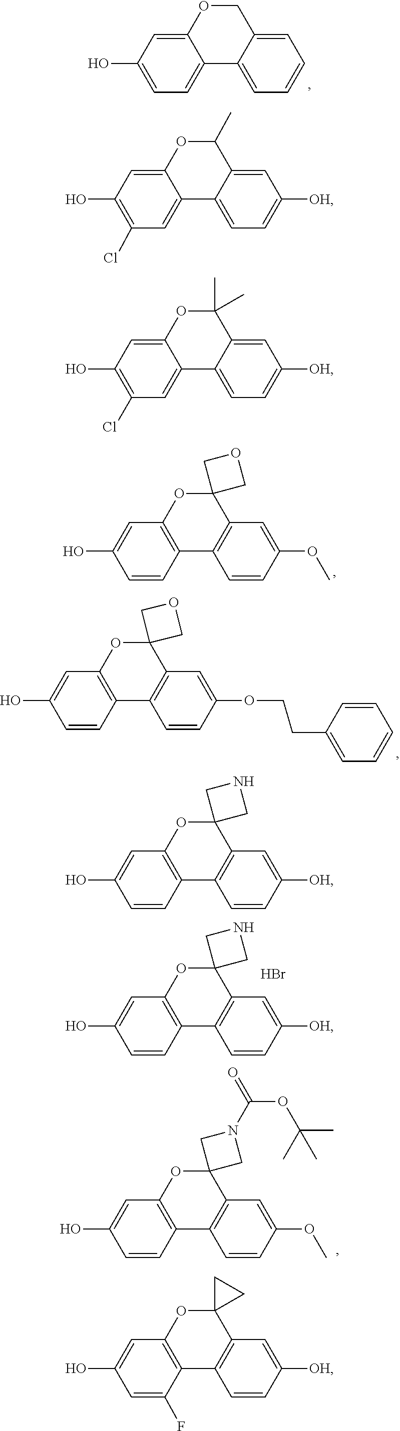

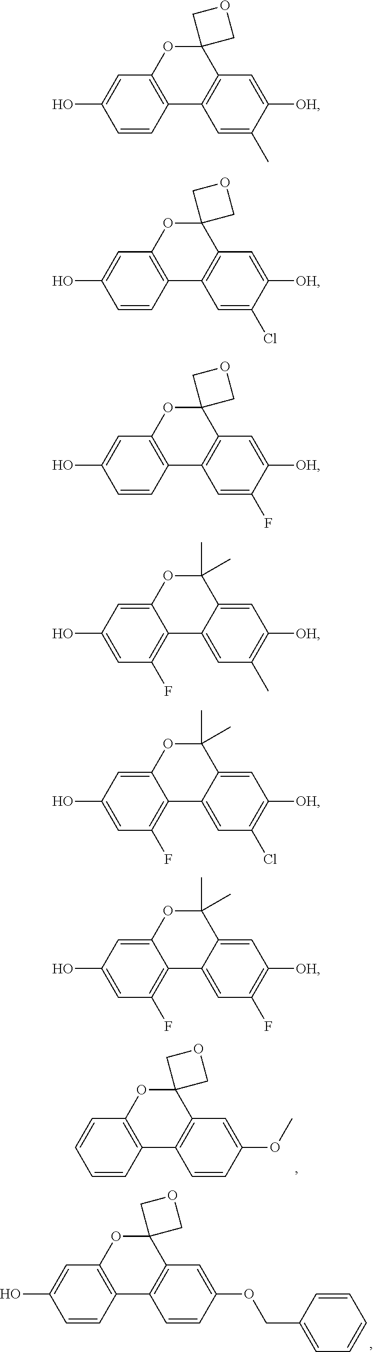

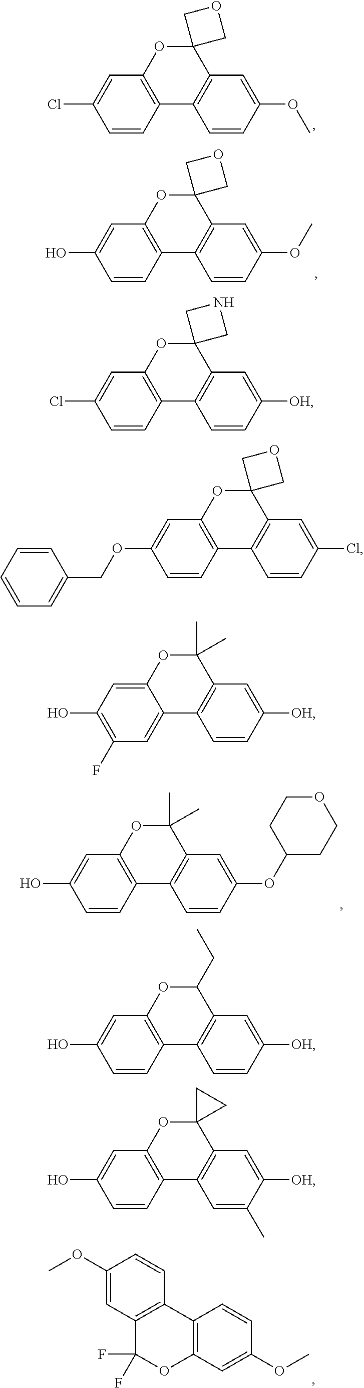

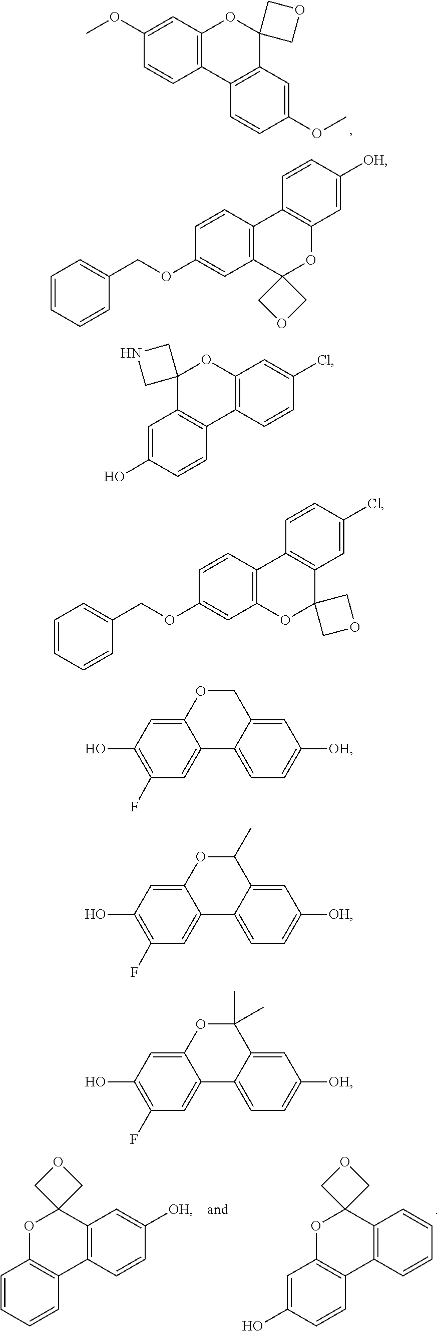

In some embodiments, the compound Formula (Id) is selected from:

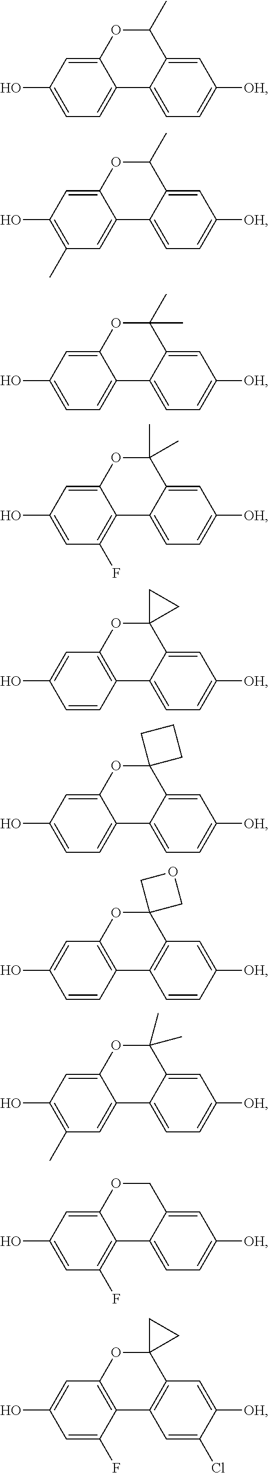

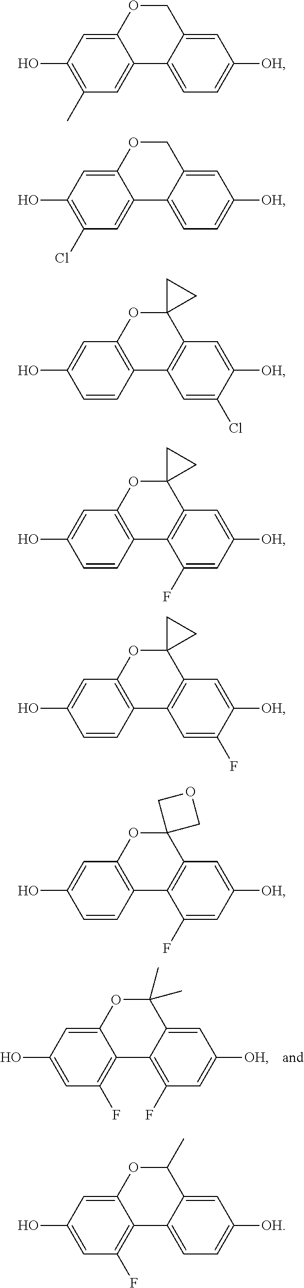

In some embodiments, the compound of Formula (Id) is selected from:

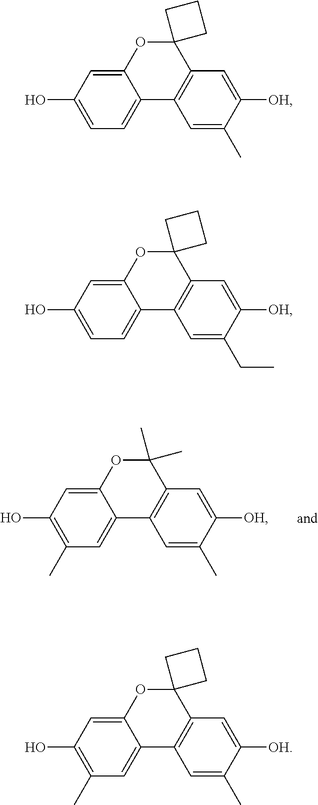 [SMILES: Cc1cc2c(cc1O)C1(CCC1)Oc1cc(O)ccc1-2 ; CCc1cc2c(cc1O)C1(CCC1)Oc1cc(O)ccc1-2 ; Cc1cc2c(cc1O)OC(C)(C)c1cc(O)c(C)cc1-2 ; "and" ; Cc1cc2c(cc1O)OC1(CCC1)c1cc(O)c(C)cc1-2]

Another aspect of the invention relates to a method of treating a neuromuscular disorder, a muscle disorder, heart disease, pulmonary fibrosis, liver disease, inflammatory bowel disease, cancer, or cognitive impairment, comprising administering to a subject in need thereof an effective amount of a compound of Formula (Ie),

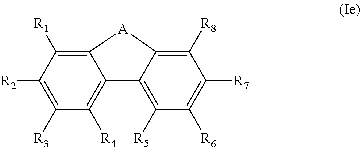

wherein
A is

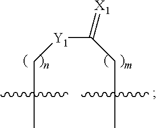

n and m are both 0; or one of n and m is 0, and the other of n and m is 1;
X₁ is O;
Y₁ is selected from NH, N—CH₃, N-t-Bu, N-cycloalkyl, and N-heterocycloalkyl;
R₁, R₂, R₃, R₆, R₇, and R₈ are independently selected from H, OH, OCH₃, OAc, NH₂, halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
R₄ and R₅ are independently selected from H, alkyl, and halogen;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of Ru is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

In some embodiments, the compound provided that no more than two of R₁, R₂, R₃, R₆, R₇, and R₈ are OH or OCH₃,
if A is

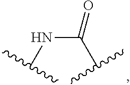 [SMILES: *NC(*)=O]

and R₁, R₃, R₄, R₅, R₆, and R₈ are each H, then R₂ and R₇ are not both OH, both OCH₃ or both OR₁₀, and
if A is

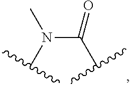 [SMILES: *C(=O)N(*)C]

and R₁, R₃, R₄, R₅, R₆, and R₈ are each H, then R₂ and R₇ are not both OR₁₀.

In some embodiments, wherein n and m are both 0. In other embodiments, one of n and m is 0, and the other of n and m is 1.

In some embodiments, A is selected from

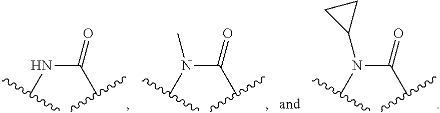

In other embodiments, A is selected from

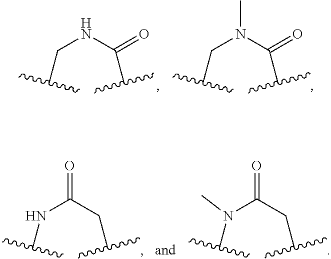

In some embodiments, wherein R₂ and R₇ are each OH.

In some embodiments, wherein one of R₂ and R₇ is OH and the other of R₂ and R₇ is OH is not OH. In other embodiments, R₂ and R₇ are each O-alkyl. In other embodiments, R₂ is OH and R₇ is O-alkyl; or R₂ is O-alkyl and R₇ is OH.

In some embodiments, wherein R₁, R₃, R₄, R₅, R₆, and R₈ are each H. In other embodiments, one of R₁, R₃, R₄, R₅, R₆, and R₈ is not H. In other embodiments, a compound two of R₁, R₃, R₄, R₅, R₆, and R₈ are not H. In other embodiments, one of R₁, R₃, R₄, R₅, R₆, and R₈ is alkyl or halogen. In other embodiments, two of R₁, R₃, R₄, R₅, R₆, and R₈ are alkyl or halogen.

In some embodiments, the compound of Formula (Ie) is selected from:

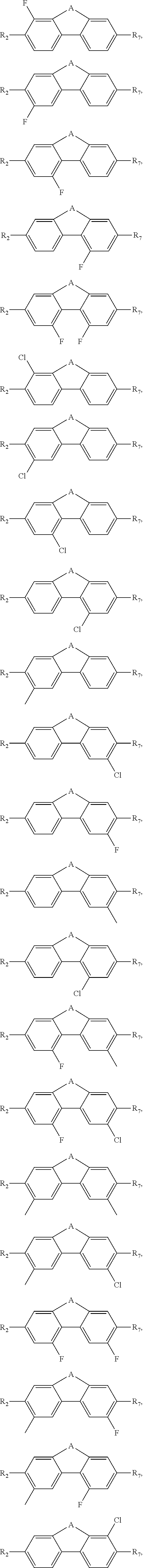

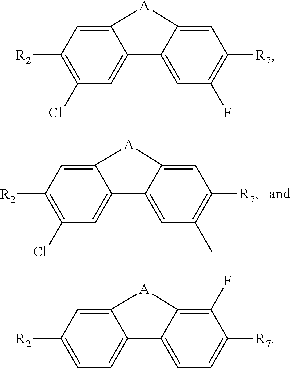

In some embodiments, the compound of Formula (Ie) is selected from:

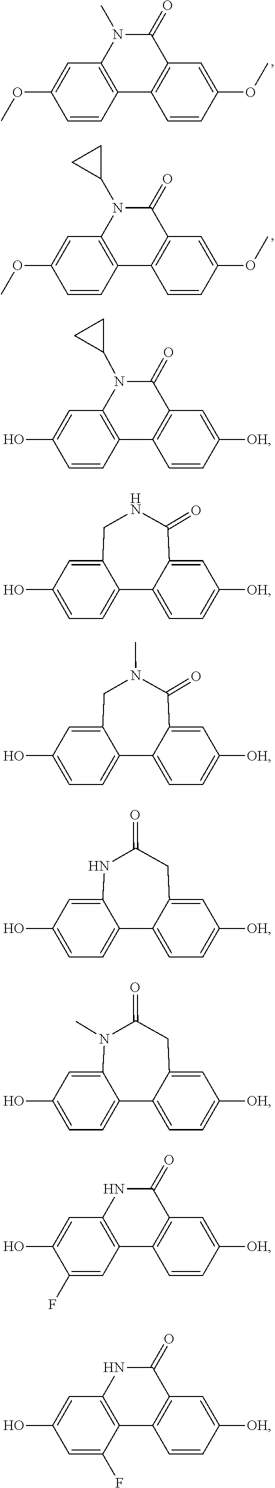

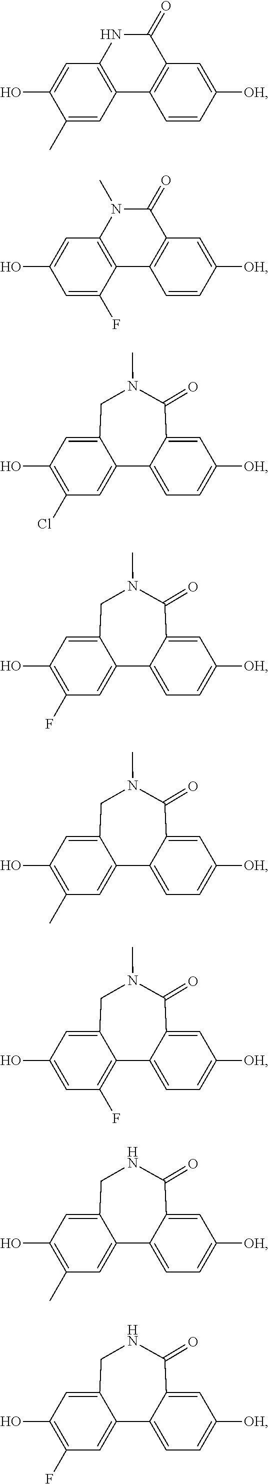

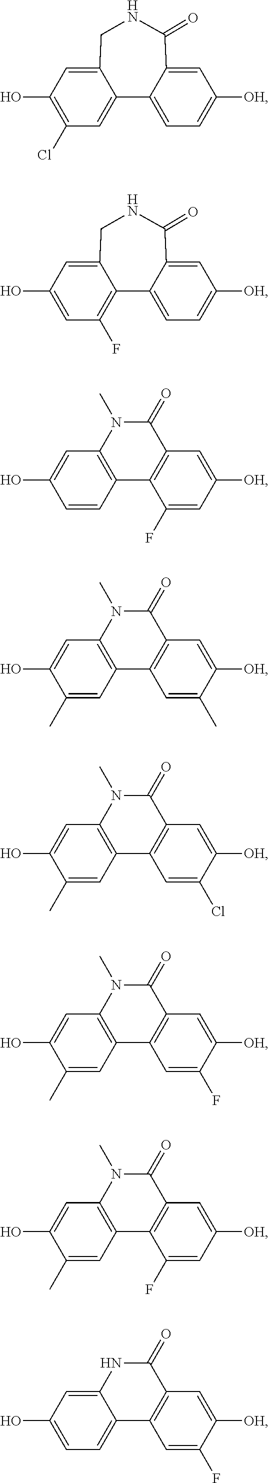

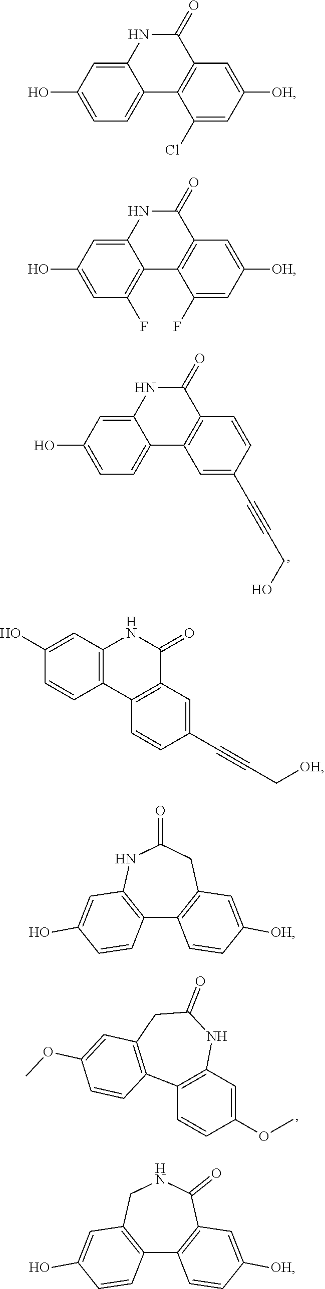

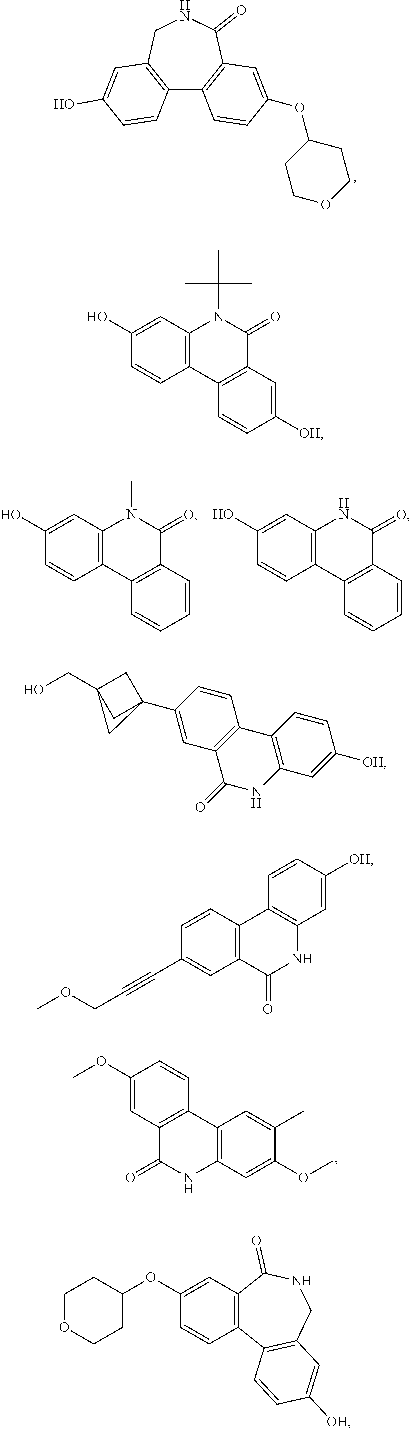

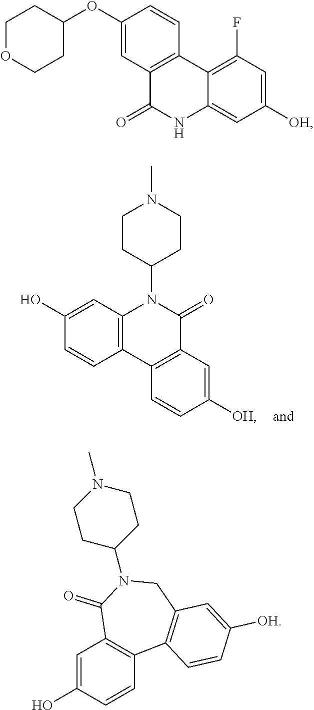 [SMILES: O=c1[nH]c2cc(O)cc(F)c2c2ccc(OC3CCOCC3)cc12 ; CN1CCC(n2c(=O)c3cc(O)ccc3c3ccc(O)cc32)CC1 ; CN1CCC(N2Cc3cc(O)ccc3-c3ccc(O)cc3C2=O)CC1]

In some embodiments, the compound of Formula (Ie) is selected from:

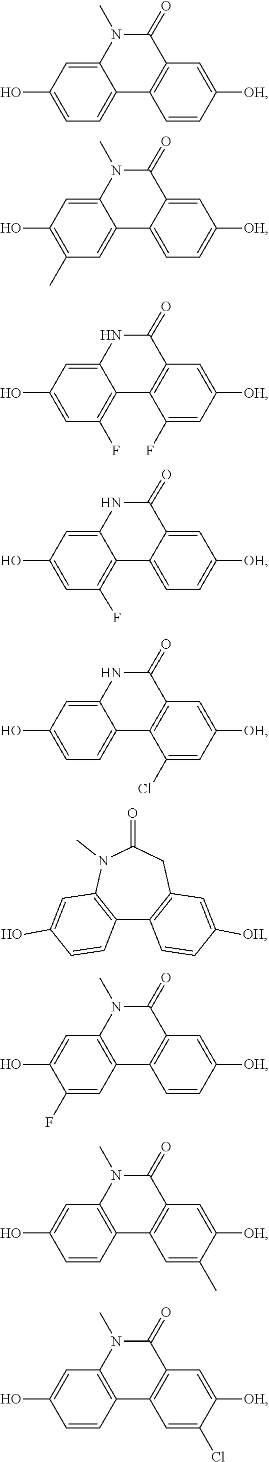

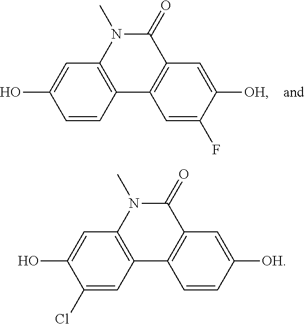

In one embodiment, the compound of Formula (Ie) is selected from:

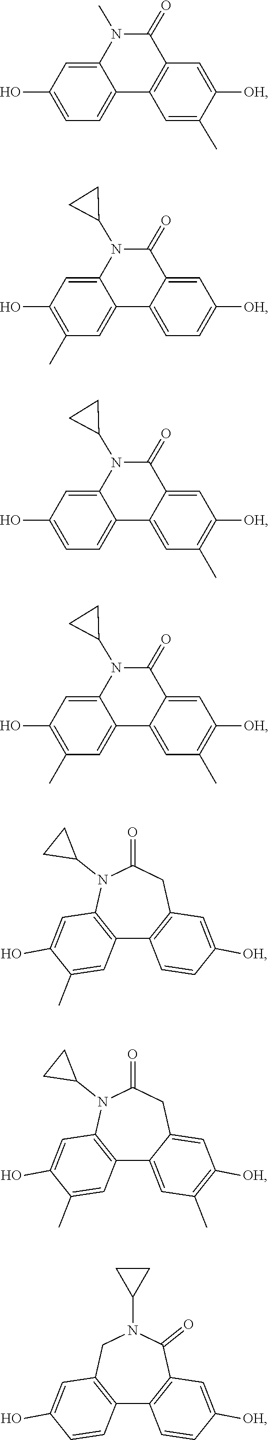

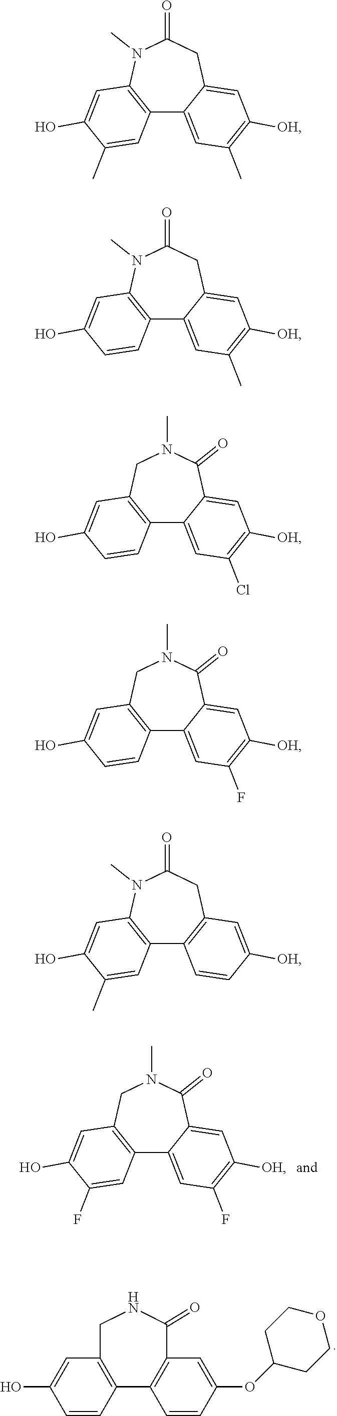

Another aspect of the invention relates to a method of treating a neuromuscular disorder, a muscle disorder, heart disease, pulmonary fibrosis, liver disease, inflammatory bowel disease, cancer, or cognitive impairment, comprising administering to a subject in need thereof an effective amount of a compound of Formula (If),

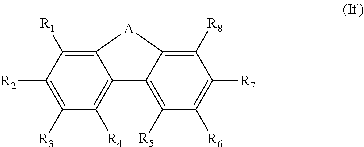

wherein
A is selected from

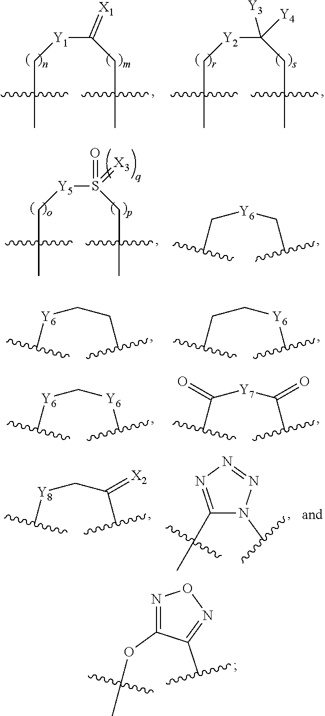

n and m are both 0; or one of n and m is 0 and the other of n and m is 1;
and p are both 0; or one of o and p is 0 and the other of o and p is 1;
q is 0 or 1;
r and s are both 0; or one of r and s is 0 and the other of r and s is 1;
X₁ and X₂ are each O;
X₃ is O or N(alkyl);
Y₁ is S;
Y₂ is selected from O, CH₂, NH, N-alkyl, S, S(O), and SO₂;
Y₃ and Y₄ are independently selected from H, halogen, OH, and alkyl, or together with the carbon to which they are bonded combine to form a cycloalkyl or cycloheteroalkyl;
Y₅ is selected from CH₂, NH, N-alkyl, N-arylalkyl, N-cycloalkyl, and N-heterocycloalkyl;
Each occurrence of Y₆ is independently selected from O, S, S(O), SO₂, NH, N-alkyl, N-alkylaryl, and N-cycloalkyl;
Y₇ is selected from O, NH and N-alkyl;
Y₈ is selected from O and S;
R₁, R₂, R₃, R₆, R₇, and R₈ are independently selected from H, OH, OCH₃, OAc, NH₂, halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁SO₂R₁₂,
R₄ and R₅ are independently selected from H, alkyl, and halogen;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

In some embodiments, the compound provided that if Y₂ is CH₂, one of Y₃ or Y₄ is not H, or Y₃ or Y₄ together with the carbon to which they are bonded combine to form a cycloalkyl or heterocycloalkyl, and if Y₂ is O, then one of r and s is 0 and the other of r and s is 1.

In some embodiments, A is

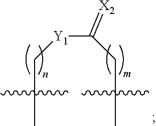

and n and m are both 0. In other embodiments, A is

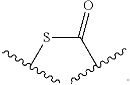 [SMILES: *SC(*)=O]

In some embodiments, A is

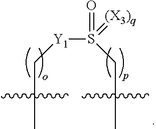

In other embodiments, A is selected from

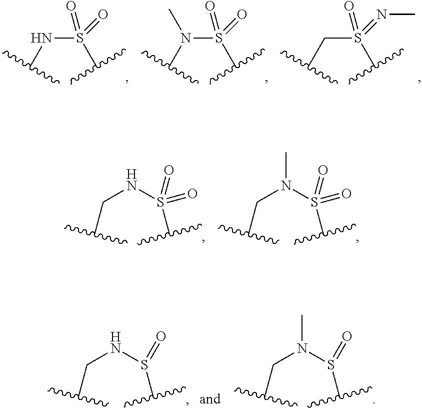

In some embodiments, A is

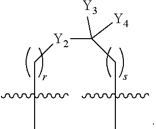

In other embodiments, A is selected from

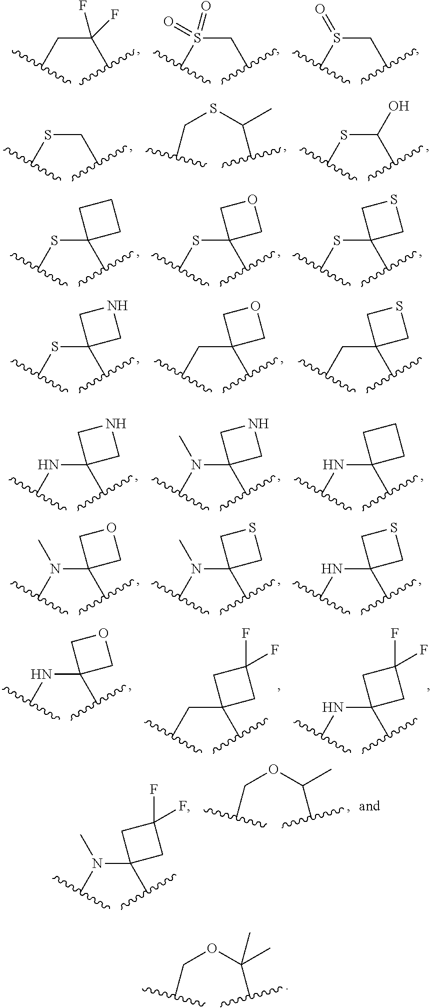

In some embodiments, A is selected from

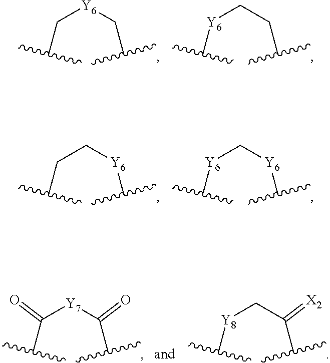

In other embodiments, A is selected from

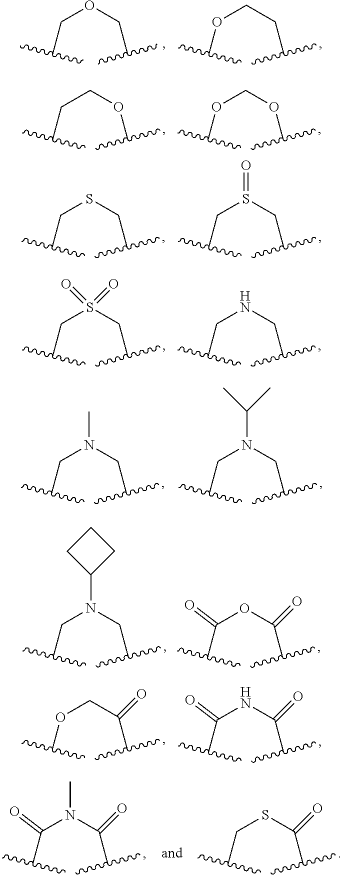

In some embodiments, R₂ and R₇ are each OH. In other embodiments, one of R₂ and R₇ is OH and the other of R₂ and R₇ is OH is not OH. In other embodiments, R₂ and R₇ are each O-alkyl. In other embodiments, R₂ is OH and R₇ is O-alkyl; or R₂ is O-alkyl and R₇ is OH.

In some embodiments, R₁, R₃, R₄, R₅, R₆, and R₈ are each H. In other embodiments, one of R₁, R₃, R₄, R₅, R₆, and R₈ is not H. In other embodiments, two of R₁, R₃, R₄, R₅, R₆, and R₈ are not H. In other embodiments, one of R₁, R₃, R₄, R₅, R₆, and R₈ is alkyl or halogen. In other embodiments, two of R₁, R₃, R₄, R₅, R₆, and R₈ are alkyl or halogen.

In some embodiments, the compound of Formula (If) is selected from:

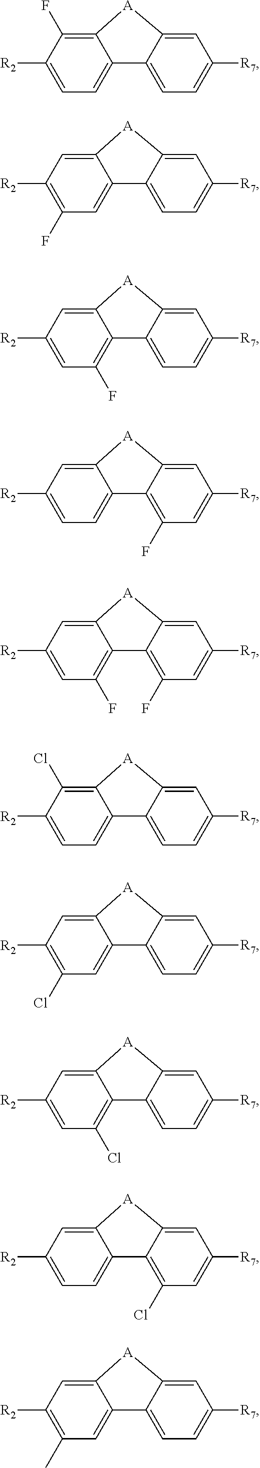

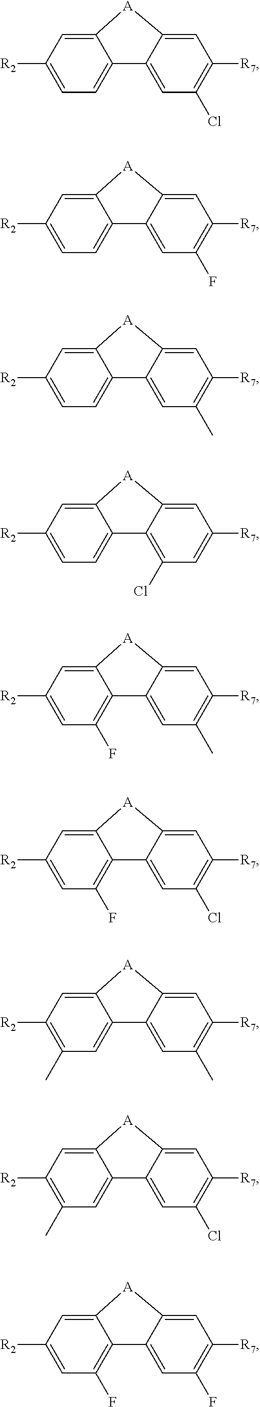

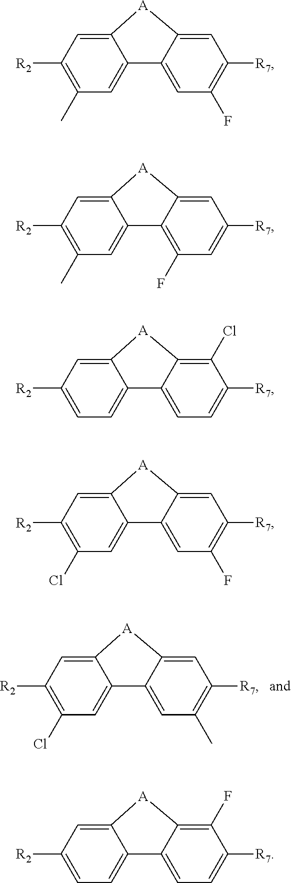

In some embodiments, the compound of Formula (If) is selected from:

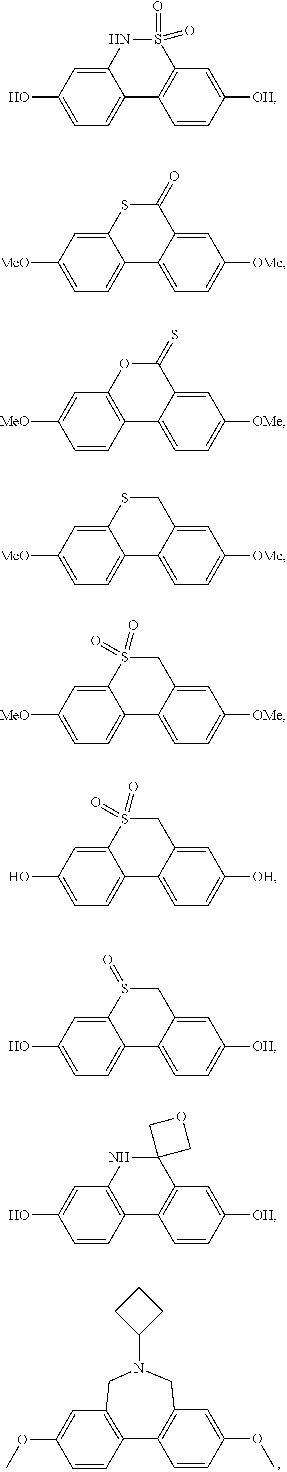

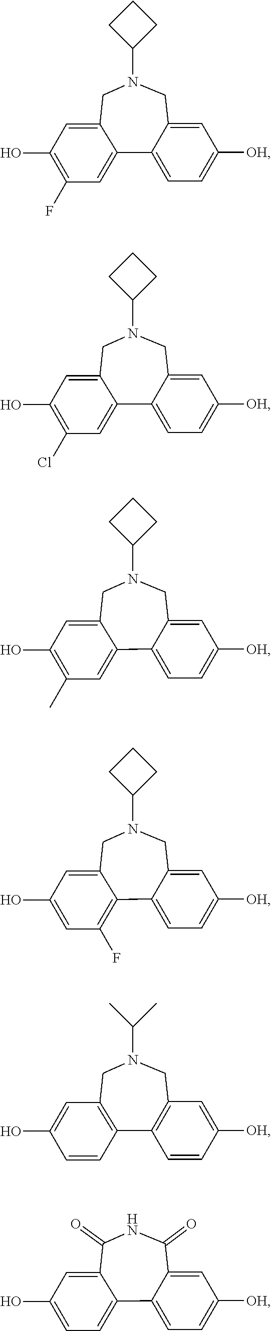

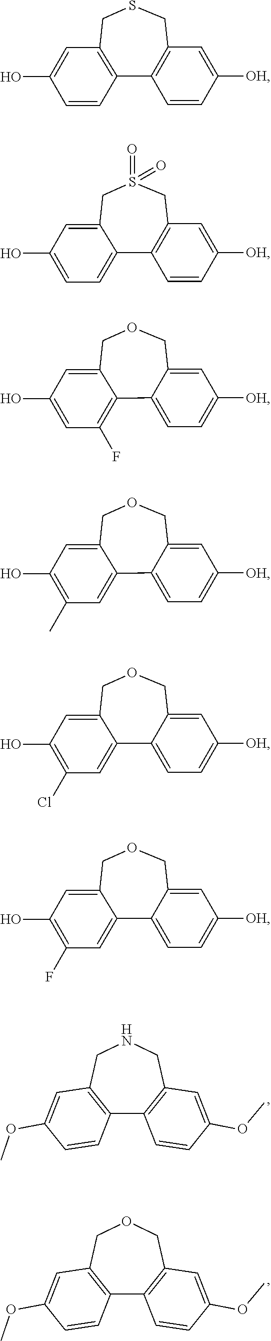

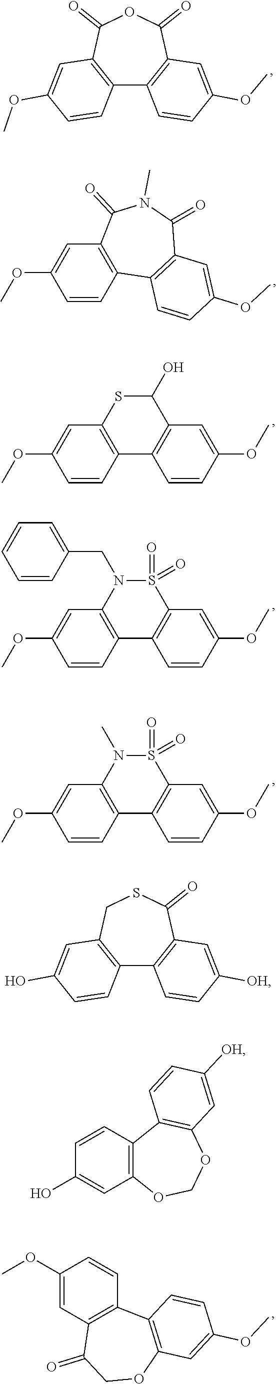

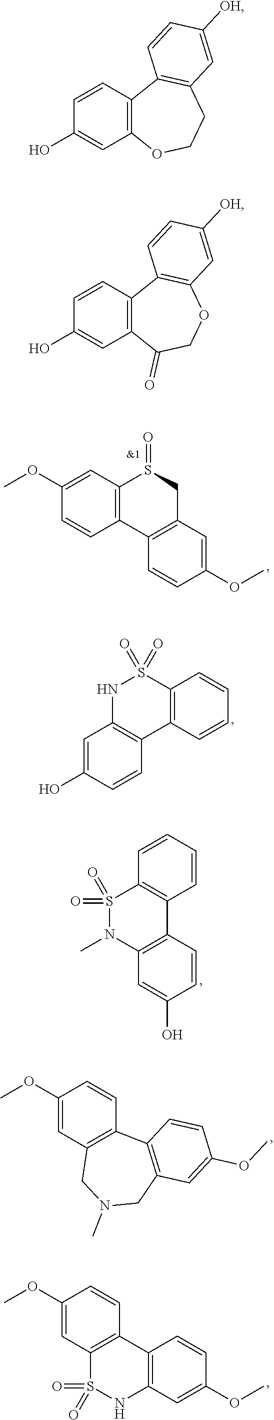

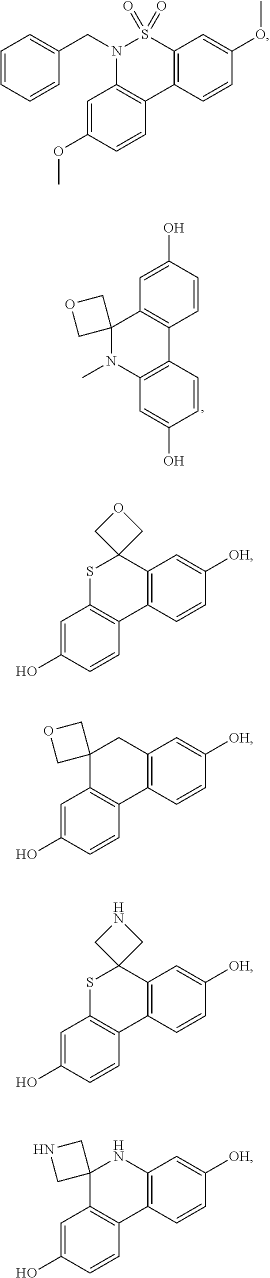 [SMILES: COc1ccc2c(c1)N(Cc1ccccc1)S(=O)(=O)c1cc(OC)ccc1-2 ; CN1c2cc(O)ccc2-c2ccc(O)cc2C12COC2 ; Oc1ccc2c(c1)SC1(COC1)c1cc(O)ccc1-2 ; Oc1ccc2c(c1)CC1(COC1)c1cc(O)ccc1-2 ; Oc1ccc2c(c1)SC1(CNC1)c1cc(O)ccc1-2 ; Oc1ccc2c(c1)NC1(CNC1)c1cc(O)ccc1-2]

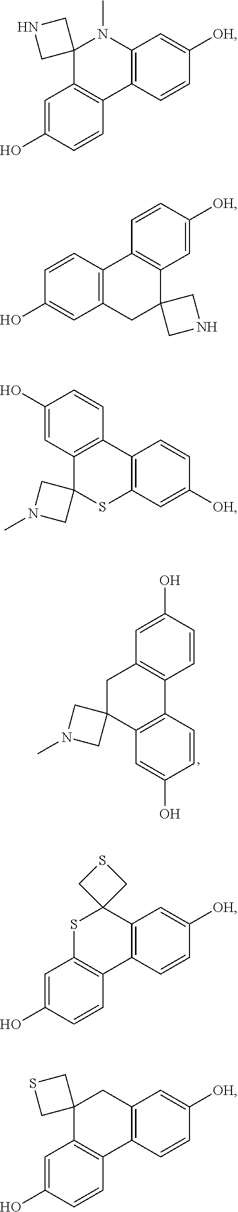 [SMILES: CN1c2cc(O)ccc2-c2ccc(O)cc2C12CNC2 ; Oc1ccc2c(c1)CC1(CNC1)c1cc(O)ccc1-2 ; CN1CC2(C1)Sc1cc(O)ccc1-c1ccc(O)cc12 ; CN1CC2(Cc3cc(O)ccc3-c3ccc(O)cc32)C1 ; Oc1ccc2c(c1)SC1(CSC1)c1cc(O)ccc1-2 ; Oc1ccc2c(c1)CC1(CSC1)c1cc(O)ccc1-2]

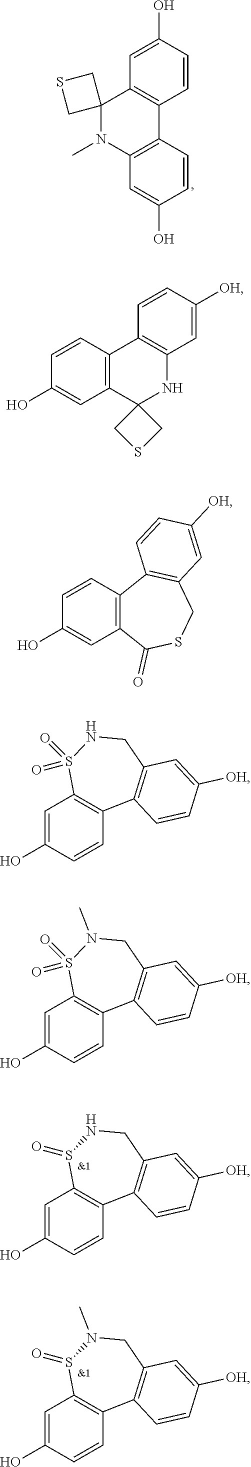

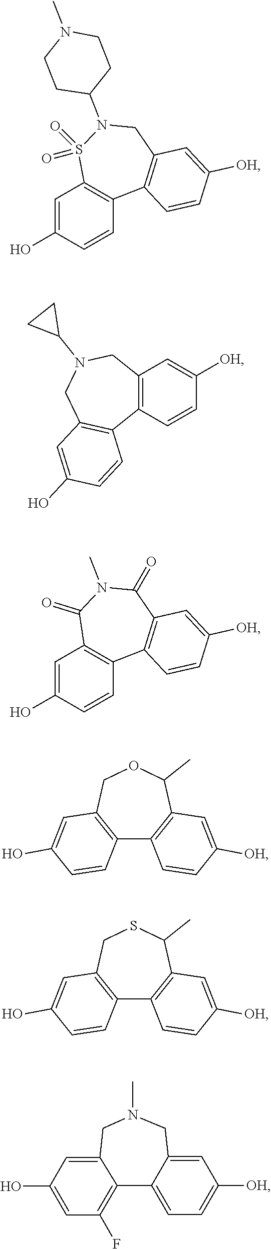

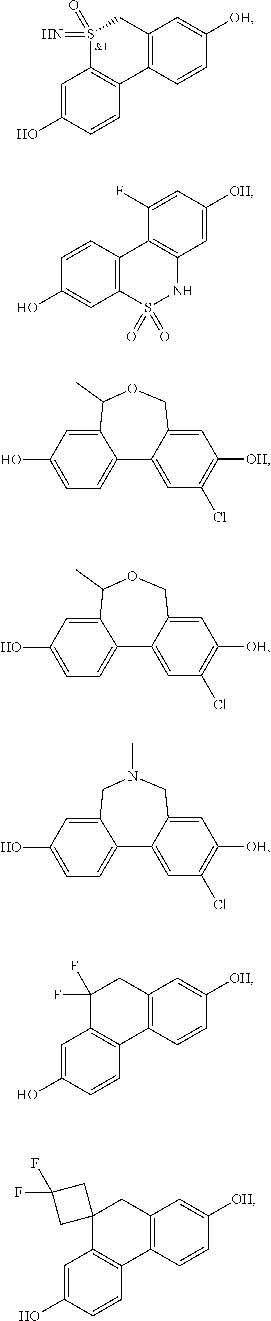

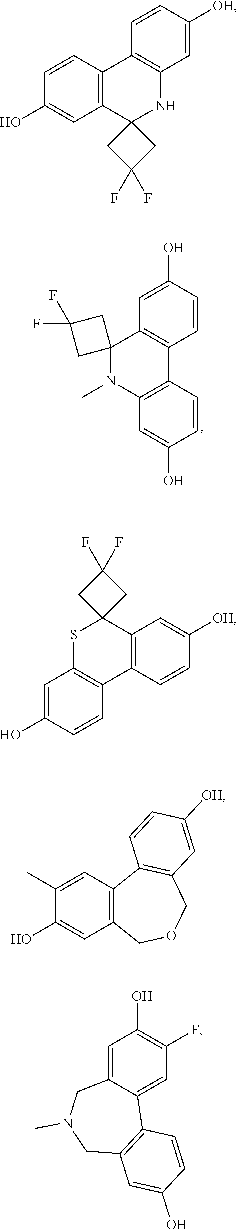 [SMILES: Oc1ccc2c(c1)NC1(CC(F)(F)C1)c1cc(O)ccc1-2 ; CN1c2cc(O)ccc2-c2ccc(O)cc2C12CC(F)(F)C2 ; Oc1ccc2c(c1)SC1(CC(F)(F)C1)c1cc(O)ccc1-2 ; Cc1cc2c(cc1O)COCc1cc(O)ccc1-2 ; CN1Cc2cc(O)ccc2-c2cc(F)c(O)cc2C1]

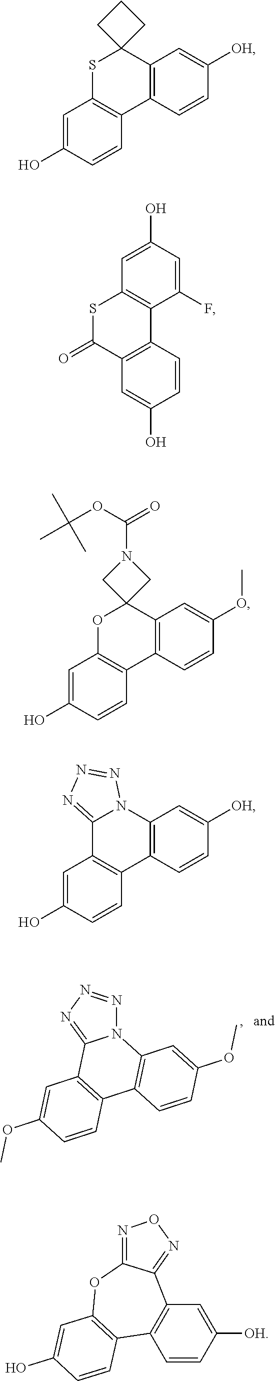

In some embodiments, the compound of Formula (If) is selected from:

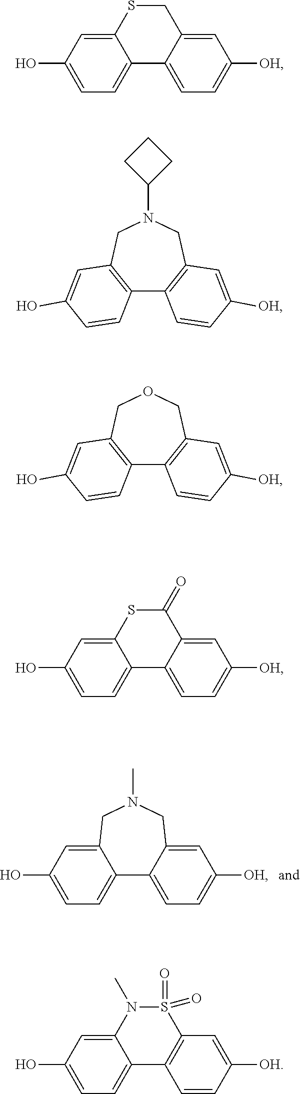 [SMILES: Oc1ccc2c(c1)CSc1cc(O)ccc1-2 ; Oc1ccc2c(c1)CN(C1CCC1)Cc1cc(O)ccc1-2 ; Oc1ccc2c(c1)COCc1cc(O)ccc1-2 ; O=c1sc2cc(O)ccc2c2ccc(O)cc12 ; CN1Cc2cc(O)ccc2-c2ccc(O)cc2C1 ; CN1c2cc(O)ccc2-c2ccc(O)cc2S1(=O)=O]

Another aspect of the invention relates to a method of treating a neuromuscular disorder, a muscle disorder, heart disease, pulmonary fibrosis, liver disease, inflammatory bowel disease, cancer, or cognitive impairment, comprising administering to a subject in need thereof an effective amount of a compound of Formula (Ih),

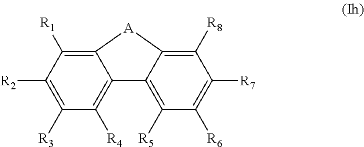

wherein
A is selected from

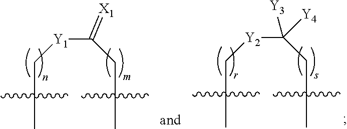

n and m are both 0; or one of n and m is 0 and the other of n and m is 1;
r and s are both 0; or one of r and s is 0 and the other of r and s is 1;
X₁ is O;
Y₁ is selected from O, NH, N-alkyl, and N-cycloalkyl;
Y₂ is O;
Y₃ and Y₄ are independently selected from H, halogen, and alkyl, or together with the carbon to which they are bonded combine to form a cycloalkyl or cycloheteroalkyl;
R₁, R₄, R₅ and R₈ are independently selected from H and halogen;
R₃ and R₆ are independently selected from H, CN, OH, CF₃, halogen, and alkyl; one of R₂ and R₇ is NH₂, NHCH₃, and N(CH₃)₂ and the other of R₂ and R₇ is H, halogen, OCH₃, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof

In some embodiments, the compound provided that if A is

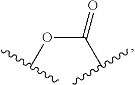 [SMILES: *OC(*)=O]

R₁, R₃, R₄, R₅, R₆, and R₈ are each H, and R₇ is NH₂, then R₂ is not OH.

In some embodiments, Y₁ is selected from O, NH, and N-alkyl.

In some embodiments, A is

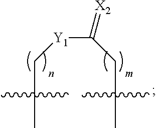

and n and m are both 0. In other embodiments, A is

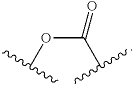 [SMILES: *OC(*)=O]

In other embodiments, A is

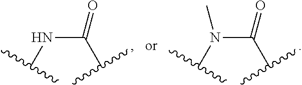

In some embodiments, A is

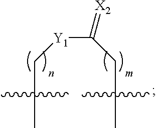

and one of n or m is 0 and the other of n or m is 1. In other embodiments, A is

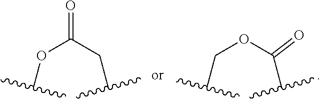 [SMILES: *CC(=O)O* ; *COC(*)=O]

In other embodiments, A is selected from

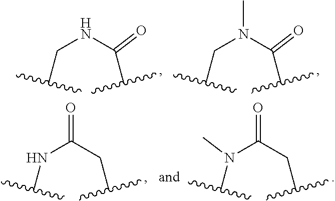

In some embodiments, A is

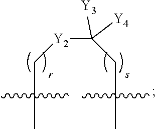

and r and s are both 0. In other embodiments, A is selected from,

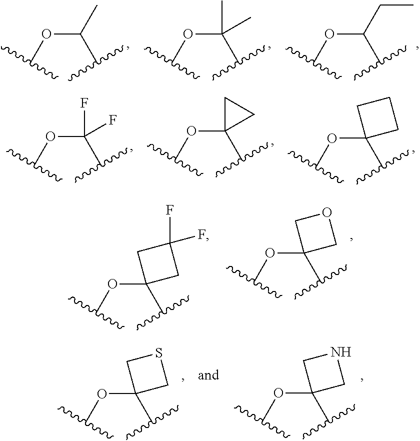

In other embodiments, A is selected from

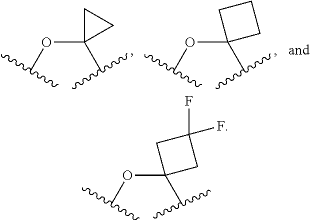

In some embodiments, wherein R₂ is selected from NH₂, NHCH₃, and N(CH₃)₂.

In some embodiments, R₇ is selected from H, OH, halogen, O-alkyl, and haloalkyl.

In some embodiments, R₇ is selected from alkynyl-R₉ and OR₁₀; R₉ is OH; and R₁₀ is alkyl-heterocycloalkyl.

In some embodiments, wherein R₁, R₃, R₄, R₅, R₆, and R₈ are each H. In other embodiments, one of R₁, R₃, R₄, R₅, R₆, and R₈ is not H. In other embodiments, two of R₁, R₃, R₄, R₅, R₆, and R₈ are not H. In other embodiments, one of R₁, R₃, R₄, R₅, R₆, and R₈ is alkyl or halogen. In other embodiments, two of R₁, R₃, R₄, R₅, R₆, and R₈ are independently alkyl or halogen.

In some embodiments, the compound of Formula (Ih) is selected from:

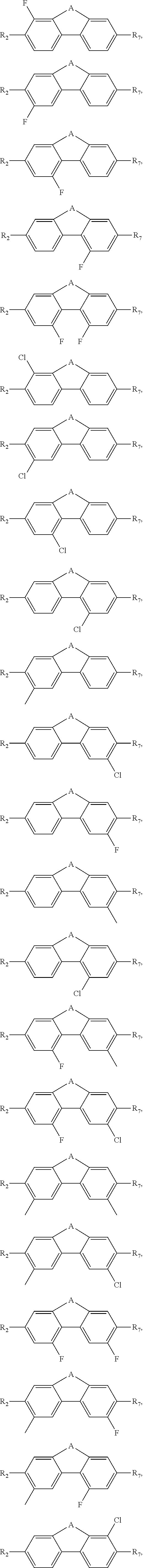

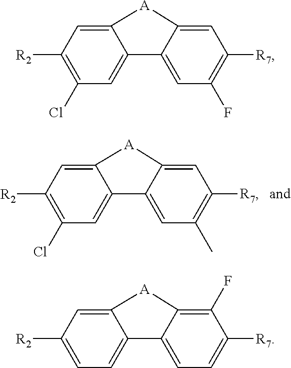

In some embodiments, the compound of Formula (Ih) is selected from:

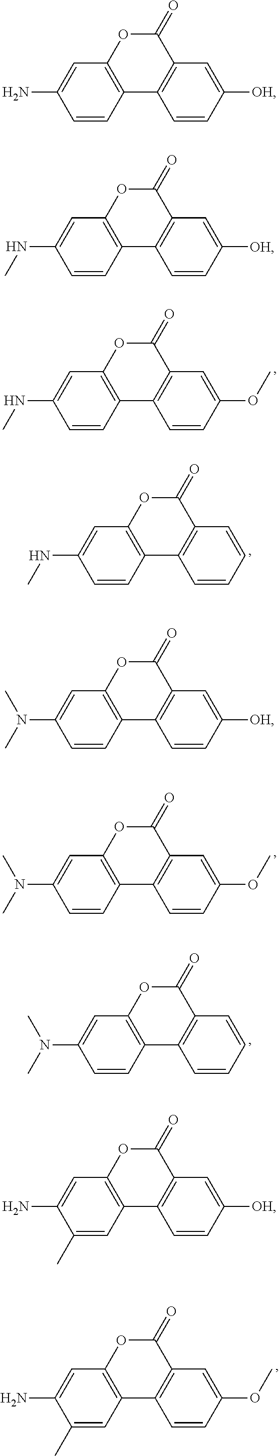

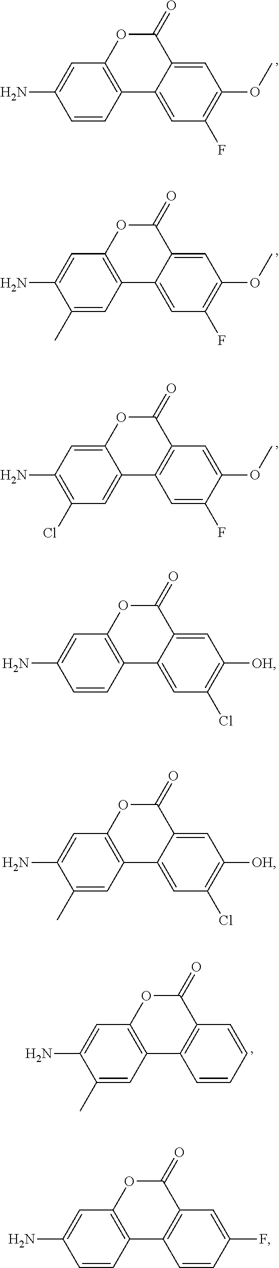

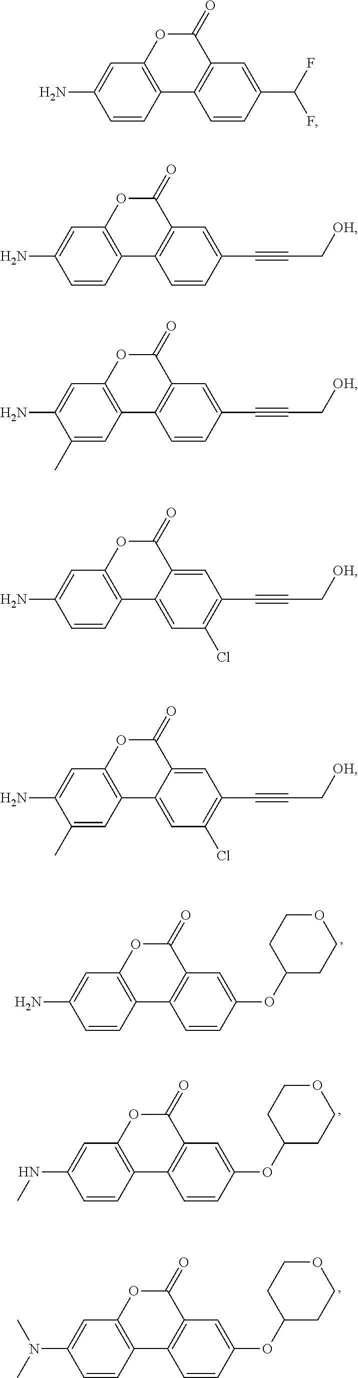

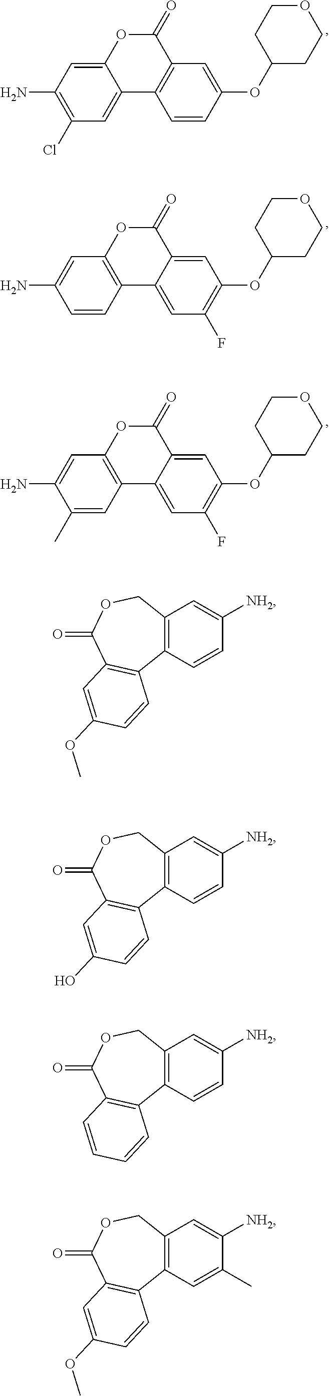

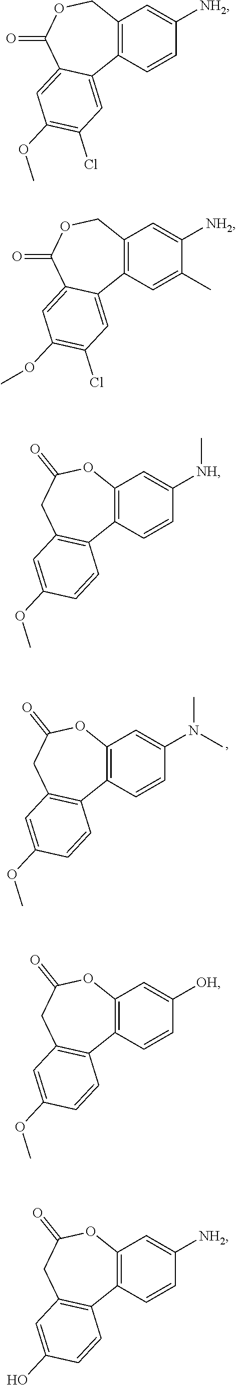

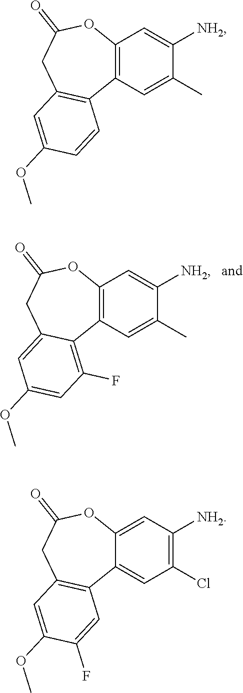

In some embodiments, the compound of Formula (Ih) is selected from:

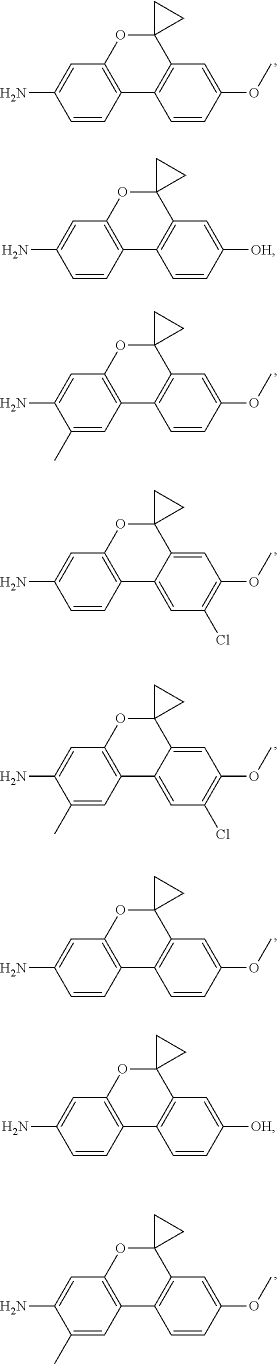

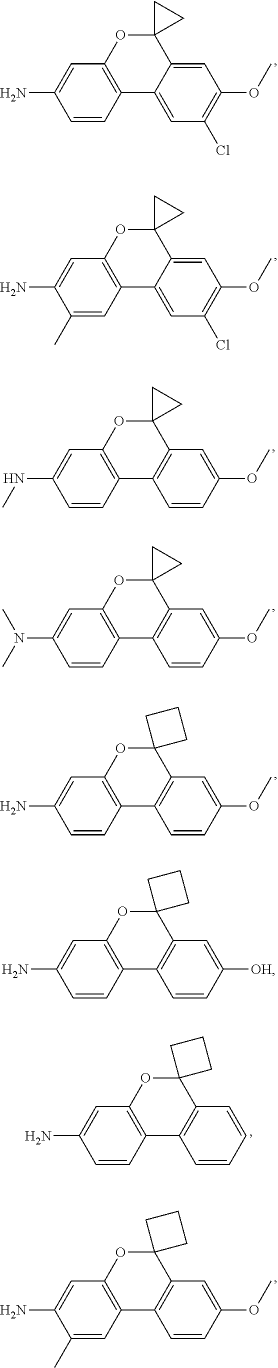

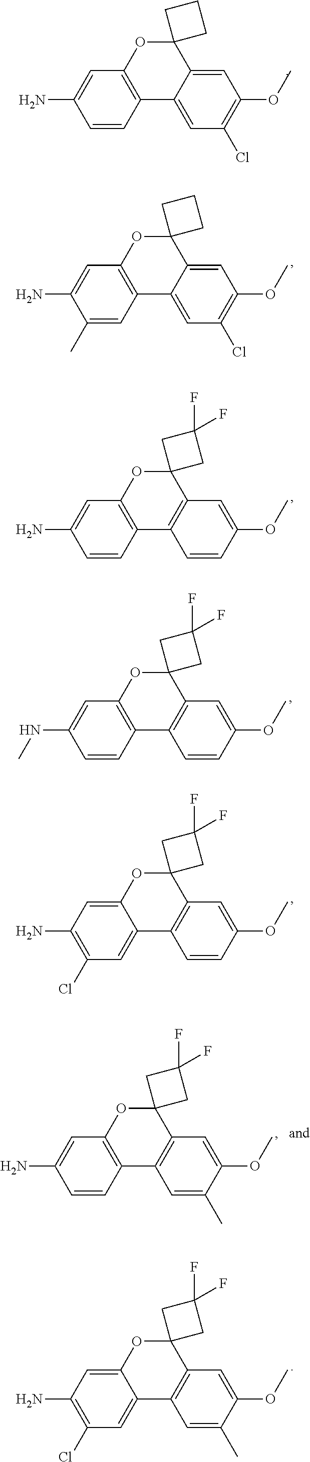

In some embodiments, the compound of Formula (Ih) is selected from:

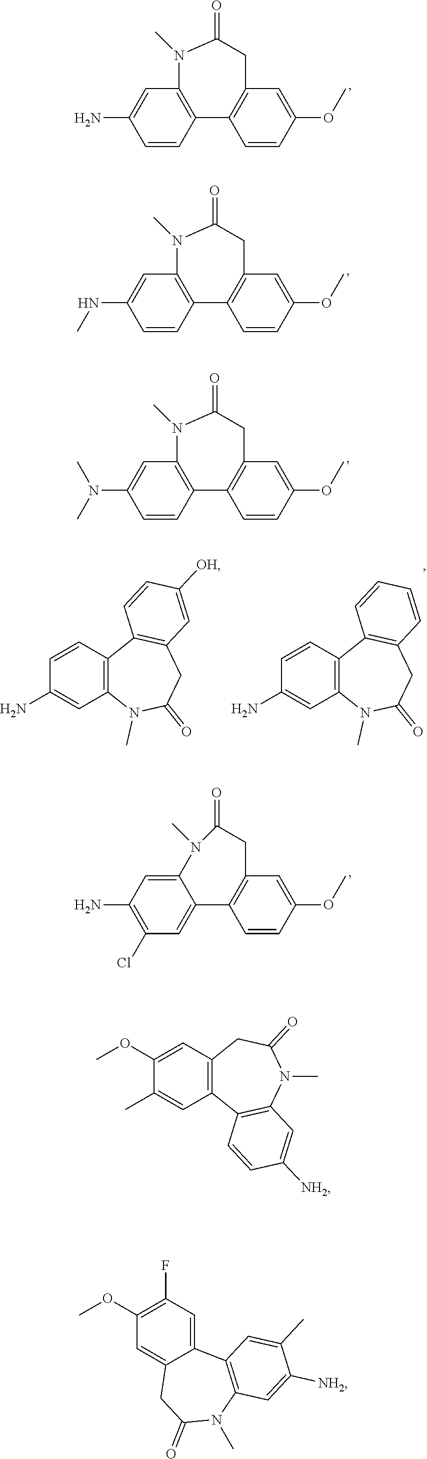

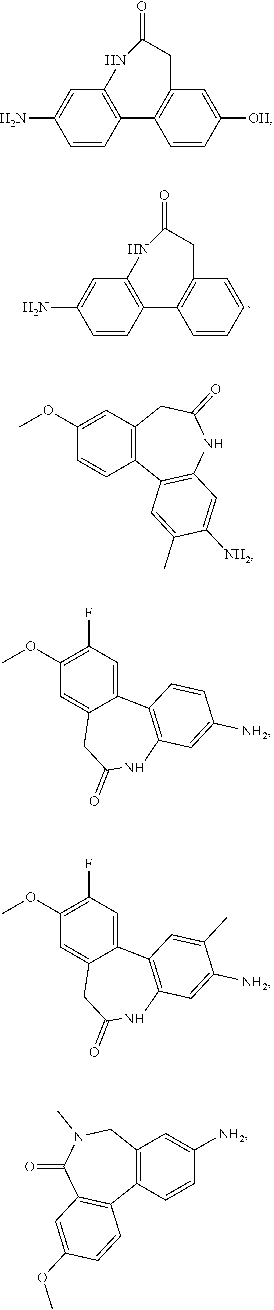 [SMILES: Nc1ccc2c(c1)NC(=O)Cc1cc(O)ccc1-2 ; Nc1ccc2c(c1)NC(=O)Cc1ccccc1-2 ; COc1ccc2c(c1)CC(=O)Nc1cc(N)c(C)cc1-2 ; COc1cc2c(cc1F)-c1ccc(N)cc1NC(=O)C2 ; COc1cc2c(cc1F)-c1cc(C)c(N)cc1NC(=O)C2 ; COc1ccc2c(c1)C(=O)N(C)Cc1cc(N)ccc1-2]

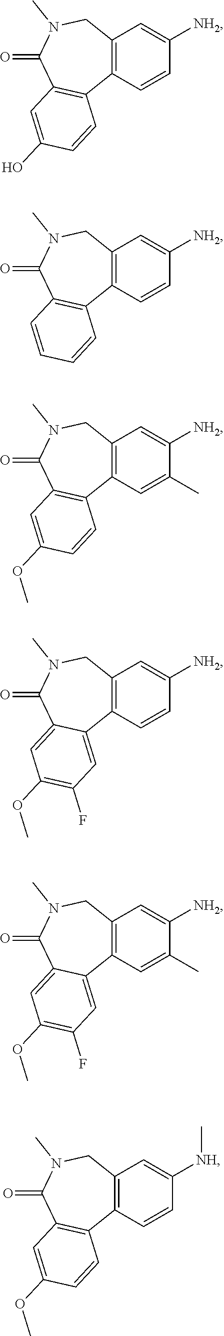

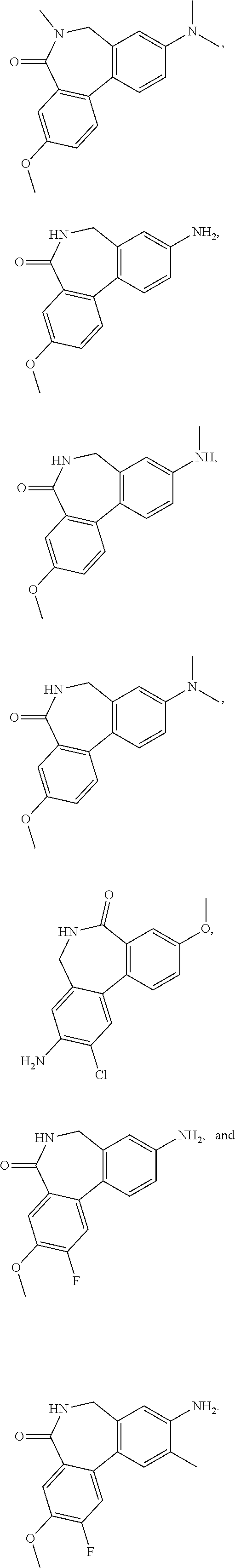

In some embodiments, the compound Formula (Ih) is selected from:

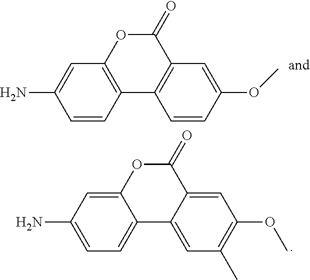

Another aspect of the invention relates to a method of treating a neuromuscular disorder, a muscle disorder, heart disease, pulmonary fibrosis, liver disease, inflammatory bowel disease, cancer, or cognitive impairment, comprising administering to a subject in need thereof an effective amount of a compound having the structure:

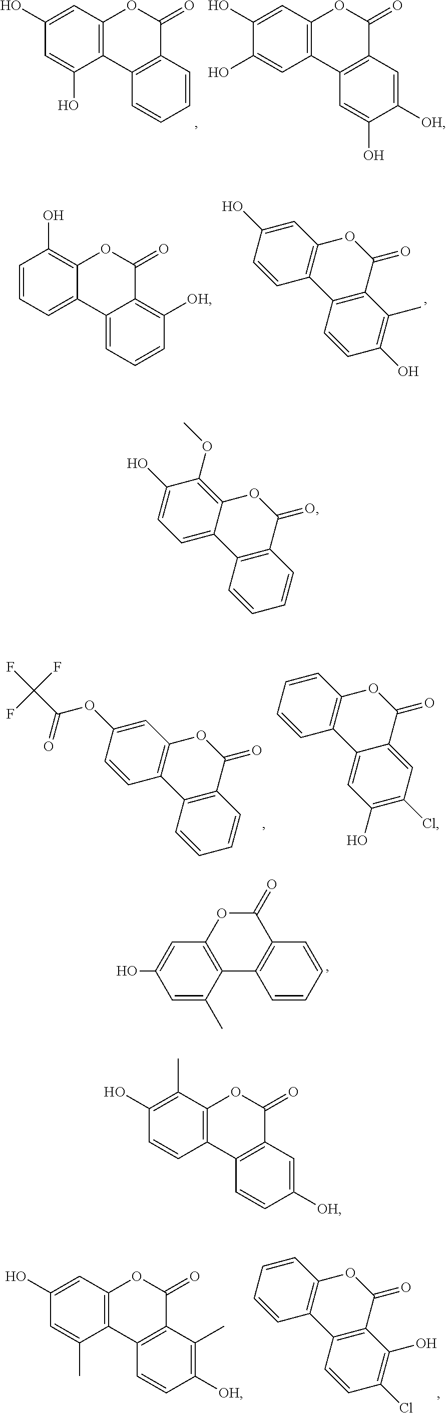

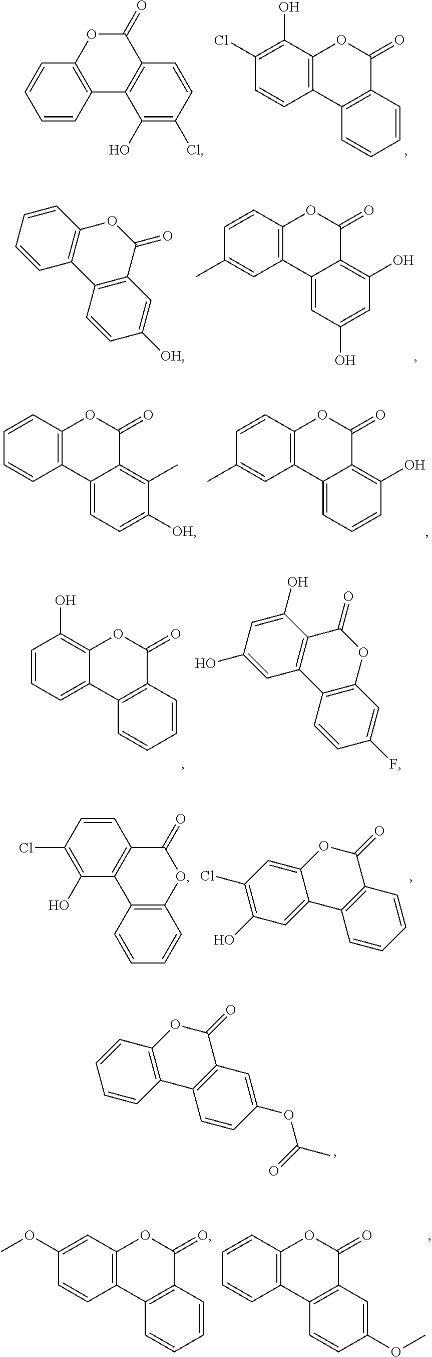

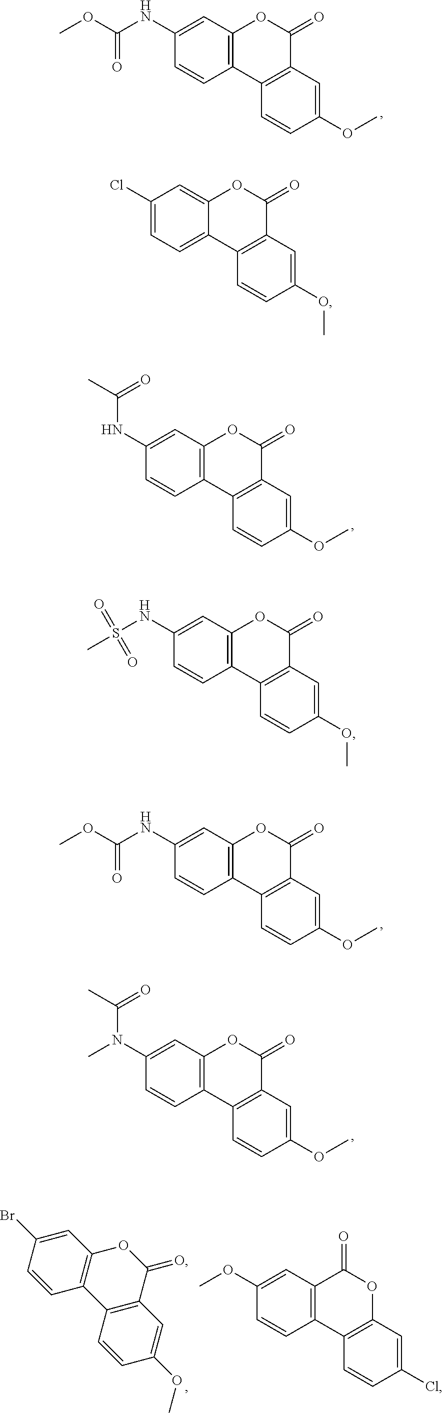

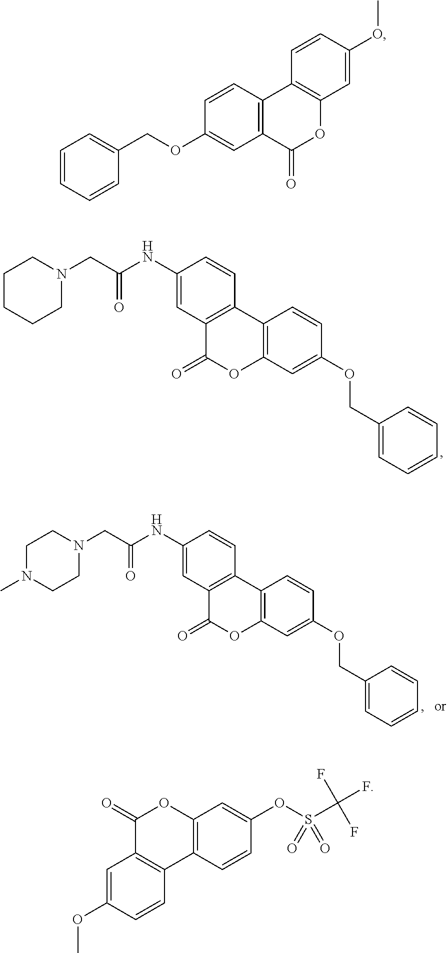

In some embodiments of any of the disclosed methods, the compound wherein R₂ and R₇ are each OH. In other embodiments, R₂ is OH and R₇ is not OH. In other embodiments, R₂ is OH and R₇ is not OCH₃. In other embodiments, R₂ is OH and R₇ is not H.

In some embodiments of any of the disclosed compounds, R₂ is OH and R₇ is OCH₃. In other embodiments, R₂ is OH and R₇ is H. In other embodiments, R₂ is OH and R₇ is alkynyl-R₉. In other embodiments, R₂ is OH and R₇ is OR₁₀. In other embodiments, R₂ is OH and R₇ is OR₁₀.

In some embodiments of any of the disclosed methods, the compound wherein R₂ is OH and R₇ is

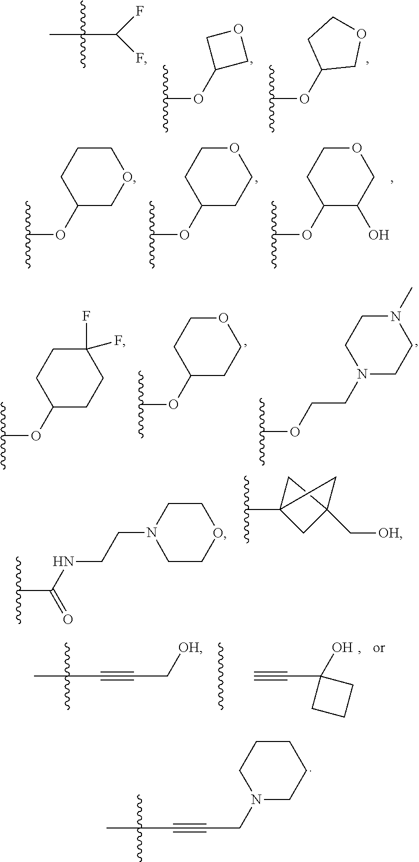

In some embodiments of any of the disclosed compounds, the compound is selected from Table 1.

TABLE 1

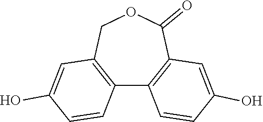 [SMILES: O=C1OCc2cc(O)ccc2-c2ccc(O)cc21]

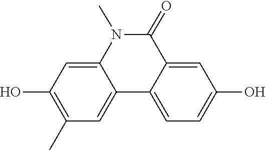 [SMILES: Cc1cc2c3ccc(O)cc3c(=O)n(C)c2cc1O]

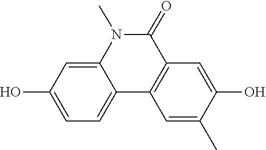 [SMILES: Cc1cc2c(cc1O)c(=O)n(C)c1cc(O)ccc21]

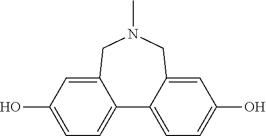 [SMILES: CN1Cc2cc(O)ccc2-c2ccc(O)cc2C1]

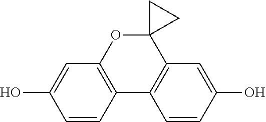 [SMILES: Oc1ccc2c(c1)OC1(CC1)c1cc(O)ccc1-2]

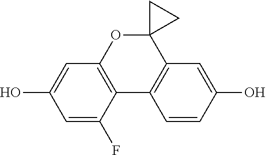 [SMILES: Oc1cc(F)c2c(c1)OC1(CC1)c1cc(O)ccc1-2]

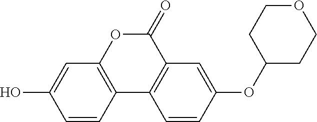 [SMILES: O=c1oc2cc(O)ccc2c2ccc(OC3CCOCC3)cc12]

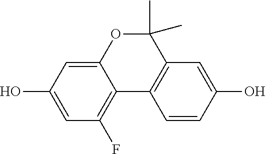 [SMILES: CC1(C)Oc2cc(O)cc(F)c2-c2ccc(O)cc21]

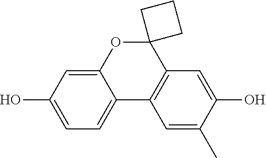 [SMILES: Cc1cc2c(cc1O)C1(CCC1)Oc1cc(O)ccc1-2]

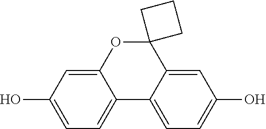 [SMILES: Oc1ccc2c(c1)OC1(CCC1)c1cc(O)ccc1-2]

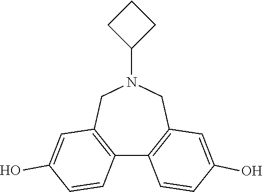 [SMILES: Oc1ccc2c(c1)CN(C1CCC1)Cc1cc(O)ccc1-2]

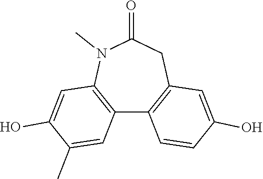 [SMILES: Cc1cc2c(cc1O)N(C)C(=O)Cc1cc(O)ccc1-2]

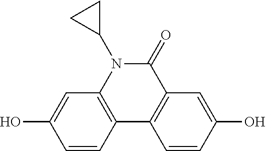 [SMILES: O=c1c2cc(O)ccc2c2ccc(O)cc2n1C1CC1]

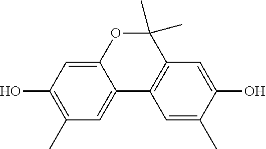 [SMILES: Cc1cc2c(cc1O)OC(C)(C)c1cc(O)c(C)cc1-2]

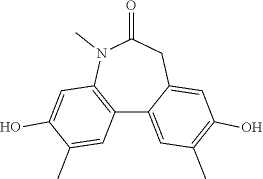 [SMILES: Cc1cc2c(cc1O)CC(=O)N(C)c1cc(O)c(C)cc1-2]

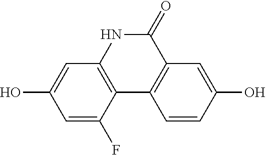 [SMILES: O=c1[nH]c2cc(O)cc(F)c2c2ccc(O)cc12]

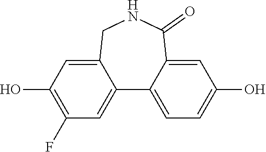 [SMILES: O=C1NCc2cc(O)c(F)cc2-c2ccc(O)cc21]

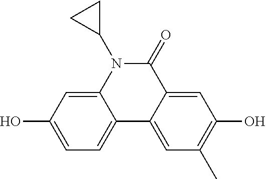 [SMILES: Cc1cc2c(cc1O)c(=O)n(C1CC1)c1cc(O)ccc21]

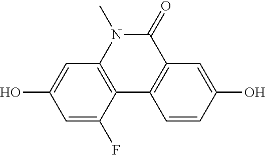 [SMILES: Cn1c(=O)c2cc(O)ccc2c2c(F)cc(O)cc21]

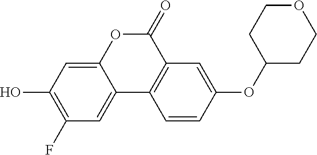 [SMILES: O=c1oc2cc(O)c(F)cc2c2ccc(OC3CCOCC3)cc12]

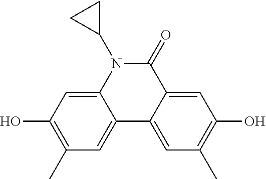 [SMILES: Cc1cc2c(cc1O)c(=O)n(C1CC1)c1cc(O)c(C)cc21]

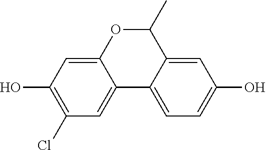 [SMILES: CC1Oc2cc(O)c(Cl)cc2-c2ccc(O)cc21]

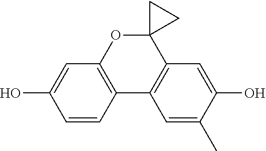 [SMILES: Cc1cc2c(cc1O)C1(CC1)Oc1cc(O)ccc1-2]

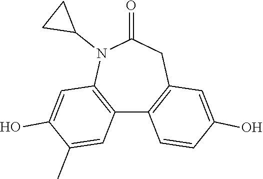 [SMILES: Cc1cc2c(cc1O)N(C1CC1)C(=O)Cc1cc(O)ccc1-2]

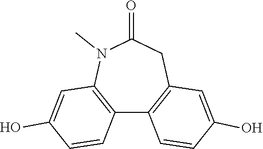 [SMILES: CN1C(=O)Cc2cc(O)ccc2-c2ccc(O)cc21]

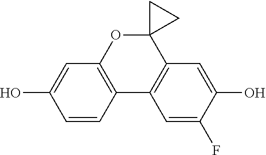 [SMILES: Oc1ccc2c(c1)OC1(CC1)c1cc(O)c(F)cc1-2]

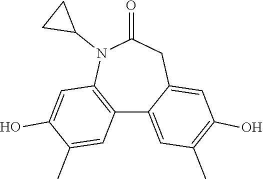 [SMILES: Cc1cc2c(cc1O)CC(=O)N(C1CC1)c1cc(O)c(C)cc1-2]

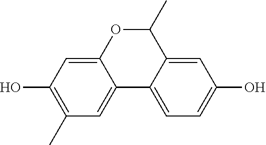 [SMILES: Cc1cc2c(cc1O)OC(C)c1cc(O)ccc1-2]

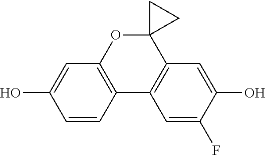 [SMILES: Oc1ccc2c(c1)OC1(CC1)c1cc(O)c(F)cc1-2]

In some embodiments of any of the disclosed methods, the compound are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O—(C₁-C₄)alkyl can be suitably deuterated (e.g., -CD₃, -OCD₃).

Any compound of the invention can also be radiolabed for the preparation of a radiopharmaceutical agent.

In one embodiment of any one of the above methods, a neuromuscular disorder, a muscle disorder, heart disease, pulmonary fibrosis, liver disease, inflammatory bowel disease, or cancer, is treated. In one embodiment of any one of the above methods, a neuromuscular disorder is treated.

In one embodiment of any one of the above methods, a muscle disorder is treated.

In one embodiment of any one of the above methods, a heart disease is treated.

In one embodiment of any one of the above methods, a pulmonary fibrosis is treated.

In one embodiment of any one of the above methods, a liver disease is treated.

In one embodiment of any one of the above methods, an inflammatory bowel disease is treated.

In one embodiment of any one of the above methods, a cancer is treated.

In one embodiment of any one of the above methods, cognitive impairment is treated.

In one embodiment of any one of the above methods, the neuromuscular disorder is Charcot-Marie-Tooth disease.

In one embodiment of any one of the above methods, the muscle disorder is hereditary inclusion body myositis, oculopharyngeal muscular dystrophy, inclusion body myopathy, Paget's disease of bone, frontotemporal gementia, or Duchenne muscular disorder.

In one embodiment of any one of the above methods, the heart disease is heart failure.

In one embodiment, the compound reduces heart failure following myocardial infarction in the subject.

In one embodiment, the compound reduces heart failure when administered to the subject following myocardial infarction in the subject.

In one embodiment, the compound reduces left ventricular systolic dysfunction following myocardial infarction in the subject.

In one embodiment, the compound reduces left ventricular systolic dysfunction when administered to the subject following myocardial infarction in the subject.

In one embodiment, ejection fraction, i.e. percentage of the total amount of blood in your heart that is pumped out with each heartbeat, in the subject is increased.

In one embodiment, fractional shortening, i.e. percentage of size reduction of the left ventricle during systole, in the subject is increased.

In one embodiment of any one of the above methods, the heart disease is a myocardial infarction, coronary artery disease (CAD), congestive heart failure (CHF), angina, stroke, arrhythmia, fibrillation, peripheral arterial disease (PAD), or a cardiac or arterial disorder.

In one embodiment of any one of the above methods, the liver disease is non-alcoholic steatohepatitis.

In one embodiment of any one of the above methods, the inflammatory bowel disease is ulcerative colitis or Crohn's disease.

In one embodiment of any one of the above methods, the cancer is responsive to immunotherapy.

In one embodiment of any one of the above methods, the compound suppresses tumor growth.

In one embodiment of any one of the above methods, the subject is concurrently be treated with cancer immunotherapy.

In one embodiment of any one of the above methods, the compound enhances the effectiveness of the cancer immunotherapy.

In one embodiment of any one of the above methods, the compound enhances the anti-tumor response of the cancer immunotherapy in the subject.

In one embodiment of any one of the above methods, the compound enhances the immune response to tumor cells in the subject.

In one embodiment of any one of the above methods, the compound promotes formation of T memory stem cells (T_{SCM}).

In one embodiment of any one of the above methods, the compound promotes formation of chimeric antigen receptor (CAR) T memory stem cells (T_{SCM}).

In one embodiment of any one of the above methods, the compound promotes anti-tumor CD8+ T cell immunity.

In one embodiment of any one of the above methods, the compound promotes anti-tumor function upon adoptive cell transfer. In one embodiment of any one of the above methods, the cancer is bladder cancer, breast cancer, cervical cancer, colorectal cancer, esophageal cancer, head and neck cancer, kidney cancer, leukemia, liver cancer, lung cancer, lymphoma, melanoma, prostate cancer, or skin cancer.

In one embodiment of any one of the above methods, the subject is concurrently being treated with an immune checkpoint inhibitor.

In one embodiment of any one of the above methods, the cancer is colorectal cancer.

In one embodiment of any one of the above methods, the subject is concurrently being treated with pembrolizumab, nivolumab, or ipilimumab.

Also provided here is a method of enhancing the effectiveness of cancer immunotherapy in a subject in need thereof, comprising administering a compound of Formula (Ia), Formula (Ic), Formula (Id), Formula (Ie), Formula (If), Formula (Ih), Formula (Ij), or Formula (Ik).

Also provided here is a method of enhancing the effectiveness of cancer immunotherapy in a subject in need thereof, comprising administering a compound of Formula (Ia), Formula (Ic), Formula (Id), Formula (Ie), Formula (If), Formula (Ih), Formula (Ij), or Formula (Ik) to the subject who is already being treated with the cancer immunotherapy.

Pharmaceutical Compositions, Routes of Administration, and Dosing

In certain embodiments, the invention is directed to a method of treating a neuromuscular disorder, a muscle disorder, heart disease, pulmonary fibrosis, liver disease, inflammatory bowel disease, or cancer, comprising administering to a subject in need thereof an effective amount of a pharmaceutical composition comprising a compound of Formula (Ia), Formula (Ic), Formula (Id), Formula (Ie), Formula (If), or Formula (Ih) and a pharmaceutically acceptable carrier.

In one embodiment of the above method, a neuromuscular disorder is treated.

In one embodiment of the above method, a muscle disorder is treated.

In one embodiment of the above method, a heart disease is treated.

In one embodiment of the above method, a pulmonary fibrosis is treated.

In one embodiment of the above method, a liver disease is treated.

In one embodiment of the above method, an inflammatory bowel disease is treated.

In one embodiment of the above method, a cancer is treated.

In one embodiment of the above method, the neuromuscular disorder is Charcot-Marie-Tooth disease.

In one embodiment of the above method, the muscle disorder is hereditary inclusion body myositis, oculopharyngeal muscular dystrophy, inclusion body myopathy, Paget's disease of bone, frontotemporal gementia, or Duchenne muscular disorder.

In one embodiment of the above method, the heart disease is heart failure.

In one embodiment of the above method, the liver disease is non-alcoholic steatohepatitis.

In one embodiment of the above method, the inflammatory bowel disease is ulcerative colitis or Crohn's disease.

In one embodiment of the above method, the cancer is bladder cancer, breast cancer, cervical cancer, colorectal cancer, esophageal cancer, head and neck cancer, kidney cancer, leukemia, liver cancer, lung cancer, lymphoma, melanoma, prostate cancer, or skin cancer

In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention.

The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### Example 1: Synthesis of Representative Compounds Employed in the Method of the Invention

All reactions were performed with oven-dried glassware and under an inert atmosphere (nitrogen) unless otherwise stated. All solvents were used as purchased unless otherwise stated. Commercial reagents were used as purchased without further purification. Organic solutions were concentrated under reduced pressure on a Büchi rotary evaporator.

Thin-layer chromatography was carried out using Merck Kieselgel 60 F254 (230-400 mesh) fluorescent treated silica and were visualized under UV light (254 and 366 nm) and/or by staining with aqueous potassium permanganate solution. 1H NMR spectra were recorded in deuterated solvents on Bruker spectrometer at 400 MHz or Nanalysis NMReady-60PRO spectrometer at 60 MHz, with residual protic solvent as the internal standard. 13C NMR spectra were recorded in deuterated solvents on Bruker spectrometer at 100 MHz, with the central peak of the deuterated solvent as the internal standard. Chemical shifts (δ) are given in parts per million (ppm) and coupling constants (J) are given in Hertz (Hz) rounded to the nearest 0.1 Hz. The 1H NMR spectra are reported as δ/ppm downfield from tetramethylsilane (multiplicity, number of protons, coupling constant J/Hz). The ¹³C NMR spectra are reported as δ/ppm. TLC-MS data was obtained on Advion Expression CMS coupled with Plate Express TLC-plate Reader. Medium pressure liquid chromatography (MPLC) was performed on a Biotage Isolera Four with built-in UV-detector and fraction collector with Interchim silica gel columns.

### 1. Synthesis of 6-Membered Urolithin a Analogues

### A) Ester “A” group analogues via the Hurtley reaction

General Procedure 1A (GP1a)

General procedure for cyclisation using NaOH and CuSO₄ (GP1a) using the synthesis of 3-hydroxy-8-methoxy-6H-benzo[c]chromen-6-one (1) as a generic example.

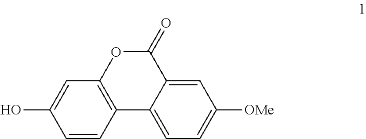 [SMILES: COc1ccc2c(c1)c(=O)oc1cc(O)ccc12]

A mixture of 2-bromo-5-methoxybenzoic acid (0.500 g, 2.16 mmol, 1.0 eq), resorcinol (0.477 g, 4.33 mmol, 2.0 eq) and sodium hydroxide (0.2 g, 4.98 mmol, 2.4 eq) in water (10 mL) was heated under reflux for 30 minutes. After the addition of copper sulphate (5% aqueous solution, 2.5 mL), the mixture was refluxed again o/n, a precipitate was formed which was filtered off and washed with 1M HCl, then dried under vacuum to afford 3-hydroxy-8-methoxy-6H-benzo[c]chromen-6-one (300 mg, 1.24 mmol 57%). ¹H NMR (400 MHz, DMSO) δ 8.27 (d, J=8.9 Hz, 1H), 8.14 (d, J=8.8 Hz, 1H), 7.67 (d, J=2.8 Hz, 1H), 7.56 (dd, J=8.8, 2.9 Hz, 1H), 6.88 (dd, J=8.7, 2.4 Hz, 1H), 6.80 (d, J=2.4 Hz, 1H), 3.95 (s, 3H).

General Procedure 1B (GP1b)

General procedure for cyclisation using Na₂CO₃ and CuI (GP1b) using the synthesis of 3-hydroxy-6-oxo-6H-benzo[c]chromen-8-carboxylic acid (2) as a generic example.

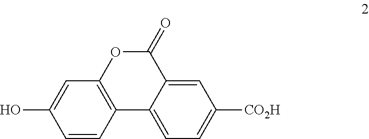 [SMILES: O=C(O)c1ccc2c(c1)c(=O)oc1cc(O)ccc12]

Resorcinol (8.9 g, 81.6 mmol, 2.0 eq) was dissolved in water and sodium carbonate (8.60 g, 81.6 mmol, 2.0 eq) was added and the mixture heated to 50° C. until everything had dissolved. Then, the acid (1000 g., 40.8 mmol, 1.0 eq) was added and stirring at 50° C. was continued for 1 h. Afterwards, CuI (0.77 g, 4.08 mmol) was added in one portion and the reaction was stirred o.n. A precipitate was formed which was filtered and washed with 1M HCl twice to get 3-hydroxy-6-oxo-6H-benzo[c]chromene-8-carboxylic acid (4.45 g, 17.4 mmol, 43%) as a beige solid. ¹H NMR (400 MHz, DMSO) δ 13.31 (s, 1H), 10.52 (1H), 8.65 (s, 1H), 8.35 (d, J=5.9 Hz, 1H), 8.29 (s, 1H), 8.18 (d, J=8.7 Hz, 1H), 6.85 (dd, J=8.7, 2.3 Hz, 1H), 6.75 (d, J=2.2 Hz, 1H).

### Synthesis of 8-bromo-3-hydroxy-6H-benzo[c]chromen-6-one (3)

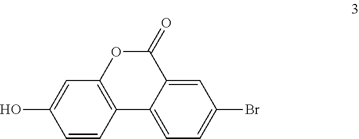 [SMILES: O=c1oc2cc(O)ccc2c2ccc(Br)cc12]

The compound was prepared according to GP1a starting from resorcinol (3.93 g, 35.7 mmol) and 2,5-dibromobenzoic acid (5.00 g, 17.9 mmol) to afford of 8-bromo 3-hydroxy-6-benzo[c]chromen-6-one (2.14 g, 42%) as a brownish solid. ¹H NMR (400 MHz, DMSO) δ 10.44 (s, 1H), 8.21 (d, J=2.2 Hz, 1H), 8.18 (d, J=8.8 Hz, 1H), 8.12 (d, J=8.8 Hz, 1H), 8.01 (dd, J=8.7, 2.2 Hz, 1H), 6.84 (dd, J=8.7, 2.4 Hz, 1H), 6.74 (d, J=2.4 Hz, 1H).

### Synthesis of N-(3-hydroxy-6-oxo-6H-benzo[c]chromen-8-yl)acetamide (4)

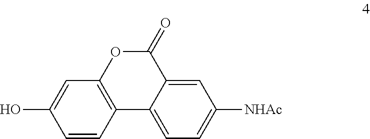 [SMILES: CC(=O)Nc1ccc2c(c1)c(=O)oc1cc(O)ccc12]

The compound was prepared according to GP1b starting from resorcinol (1.40, 12.8 mmol) and 5-acetamido-2-bromobenzoic acid (1.00 g, 3.87 mmol) to afford N-(3-hydroxy-6-oxo-6H-benzo[c]chromen-8-yl)acetamide (620 mg. 29%) as a beige solid. ¹H NMR (400 MHz, DMSO) δ 10.32 (s, 1H), 10.27 (s, 1H), 8.50 (d, J=2.2 Hz, 1H), 8.20 (d, J=8.8 Hz, 1H), 8.07 (d, J=8.7 Hz, 1H), 7.99 (dd, =8.8, 2.3 Hz, 1H), 6.83 (dd, J=8.7, 2.4 Hz, 1H), 6.74 (d, J=2.3 Hz, 1H), 2.10 s, 3H).

### Deprotection of 4 Afforded 8-amino-3-hydroxy-6H-benzo[c]chromen-6-one (5)

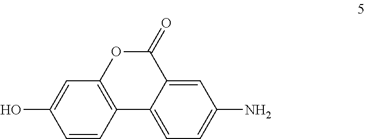 [SMILES: Nc1ccc2c(c1)c(=O)oc1cc(O)ccc12]

### Synthesis of 8-fluoro-3-hydroxy-6H-benzo[c]chromen-6-one (6)

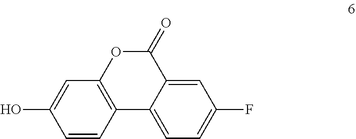 [SMILES: O=c1oc2cc(O)ccc2c2ccc(F)cc12]

The compound was prepared according to GP1a starting from resorcinol (2.01 g, 18.3 mmol) and 2-bromo-5-fluorobenzoic acid (2.00 g, 9.13 mmol) to afford of 8-fluoro-3-hydroxy-6H-benzo[c]chromen-6-one (1.00 g, 48%) as a brownish solid. ¹H NMR (400 MHz, DMSO) δ 10.54 (s, 1H), 8.31 (d, J=2.2 Hz, 1H), 8.28 (d, J=8.8 Hz, 1H), 8.32 (d, J=8.8 Hz, 1H), 8.21 (dd, J=8.7, 2.2 Hz, 1H), 7.04 (dd, J=8.7, 2.4 Hz, 1H), 6.94 (d, J=2.4 Hz, 1H).

### B) Amide “A” Group Analogues

### Synthesis of 3,8-dihydroxyphenanthridin-6(5H)-one (18)

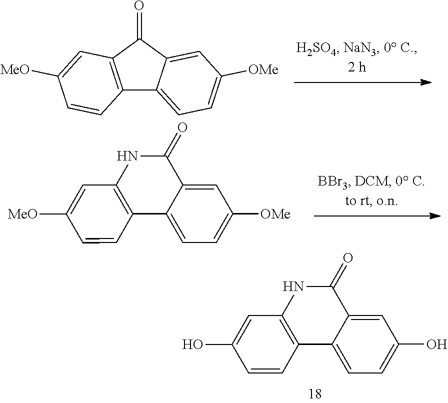

### Step 1: Synthesis of 3,8-dimethoxyphenanthridin-6(5H)-one

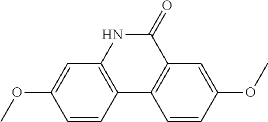

To cold sulfuric acid (10 mL) was added at 0° C. 2,7-dimethoxy-9H-fluoren-9-one (1.10 g, 4.57 mmol), and then carefully sodium azide (387 mg, 5.95 mmol). The reaction mixture was stirred at 0° C. for 3 h. EtOAc (10 mL) was added and the mixture was poured into ice water and stirred for 1 h. The brownish precipitate was filtered and the aqueous phase was extracted with EtOAc 3 times. The organic phase was dried over sodium sulfate and evaporated under vacuum. The crude was purified by MPLC (SiO₂, EtOAc/cyclohexane from 0% to 80%) to afford 3,8-dimethoxyphenanthridin-6(5H)-one (150 mg, 13%) as a brown solid. R_{f}=0.4 (EtOAc/hexane 50%). ¹H NMR (400 MHz, DMSO) δ 11.60 (s, 1H), 8.32 (d, J=8.9 Hz, 1H), 8.20 (d, J=8.7 Hz, 1H), 7.70 (d, J=2.8 Hz, 1H), 7.40 (dd, J=8.9, 2.9 Hz, 1H), 6.91-6.81 (m, 2H), 3.89 (s, 3H), 3.81 (s, 3H).

### Step 2: Synthesis of 3,8-dihydroxyphenanthridin-6(5H)-one

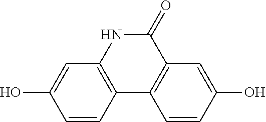 [SMILES: O=c1[nH]c2cc(O)ccc2c2ccc(O)cc12]

18 was prepared according to GP2 from 3,8-dimethoxyphenanthridin-6(5H)-one (90 mg, 0.35 mmol) and BBr₃ (1M in THF, 2.10 ml, 2.10 mmol) to afford after purification by MPLC (SiO₂, MeOH/DCM 0% to 10%) 3,8-dihydroxyphenanthridin-6(5H)-one (70 mg, 87%) as a brownish solid. R_{f}=0.2 (MeOH in DCM 10%). ¹H NMR (400 MHz, DMSO) δ 11.91-11.20 (m, 1H), 10.34-9.57 (m, 2H), 8.08 (d, J=49.2 Hz, 2H), 7.82-7.48 (m, 1H), 7.23 (s, 1H), 6.70 (d, J=27.5 Hz, 2H).

### Synthesis of 3,8-dihydroxy-5-methylphenanthridin-6(5H)-one (20)

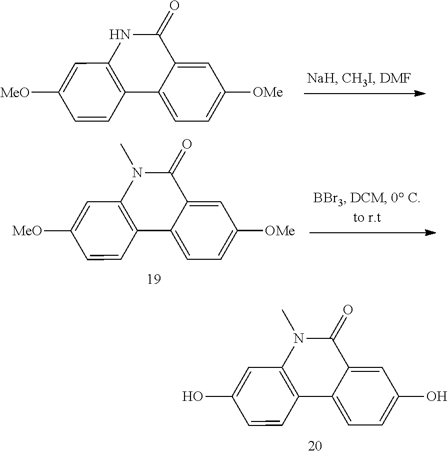

### Step 1: Synthesis of 3,8-dimethoxy-5-methylphenanthridin-6(5H)-one (19)

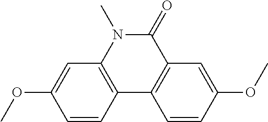

NaH (60% mineral oil dispersion, 59 mg, 1.5 mmol) was added to a solution of 3,8-dimethoxyphenanthridin-6(5H)-one (250 mg, 0.98 mmol) in DMF (10 mL) at 0° C. and the mixture was stirred for 30 min at 0° C. Then, Mel (0.122 ml, 1.96 mmol) was added and stirring continued at rt for 2 h. The reaction mixture was poured into a sat. aq. Solution of NH₄Cl and extracted with EtOAc 3 times. The combined organic phases were dried over sodium sulfate and concentrated under vacuum. The crude product was purified by MPLC (SiO₂, EtOAc/cyclohexane 0% to 40%) to afford 3,8-dimethoxy-5-methylphenanthridin-6(5H)-one (176 mg, 67%). R_{f}=0.3 eluent (EtOAc/hexane 50%). ¹H NMR (400 MHz, CDCl₃) δ 8.10 (dd, J=9.2, 8.0 Hz, 2H), 7.93 (d, J=2.8 Hz, 1H), 7.32 (dd, J=8.9, 2.9 Hz, 1H), 6.93-6.86 (m, 2H), 3.95 (s, 3H), 3.93 (s, 3H), 3.80 (s, 3H).

### Step 2: Synthesis of 3,8-dihydroxy-5-methylphenanthridin-6(5H)-one (20)

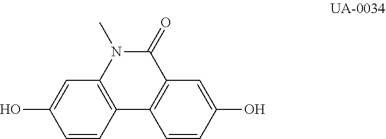 [SMILES: Cn1c(=O)c2cc(O)ccc2c2ccc(O)cc21]

20 was prepared according to GP2 starting from 3,8-dimethoxy-5-methylphenanthridin-6(5H)-one (150 mg, 0.550 mmol) to afford after purification by MPLC (SiO₂, MeOH/DCM 0% to 10%) 3,8-dihydroxy-5-methylphenanthridin-6(5H)-one (120 mg, 89%) as a beige solid. R_{f}=0.8 (MeOH/DCM 10/90). ¹H NMR (400 MHz, DMSO) δ 9.92 (s, 2H), 8.18 (d, J=8.9 Hz, 1H), 8.13 (d, J=8.8 Hz, 1H), 7.64 (d, J=2.7 Hz, 1H), 7.22 (dd, J=8.8, 2.7 Hz, 1H), 6.85 (d, J=2.3 Hz, 1H), 6.77 (dd, J=8.7, 2.3 Hz, 1H), 3.63 (s, 3H).

### Synthesis of 5-cyclopropyl-3,8-dihydroxyphenanthridin-6(5H)-one (21)

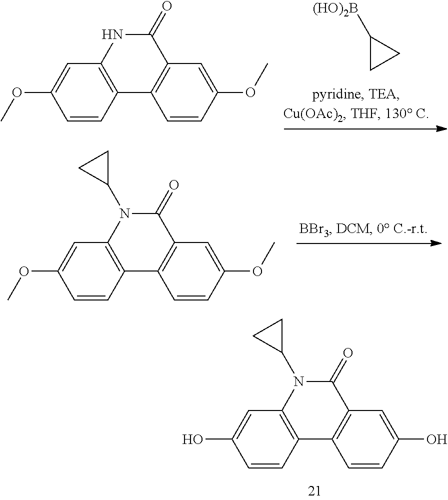

### Step 1: Synthesis of 5-cyclopropyl-3,8-dimethoxyphenanthridin-6(5H)-one

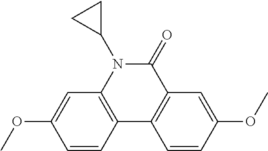

A microwave vial was charged with 3,8-dimethoxyphenanthridin-6(5H)-one (120 mg, 0.470 mmol, 1.0 eq.), cyclopropylboronic acid (121 mg, 1.41 mmol, 3.0 eq.), pyridine (355 mg, 4.23 mmol, 9.0 eq.), triethylamine (285 mg, 2.82 mmol, 6.0 eq.) and THF (2.0 mL) and the resulting mixture was degassed with a N₂ balloon for 10 min at r.t. Then, Cu(OAc)₂ (171 mg, 0.940 mmol, 2.0 eq.) was added in one portion and the vial was closed and put into a preheated 130° C. oil-bath for 2 h. Upon complete consumption of the starting material the reaction was allowed to cool to r.t., and subsequently quenched with water, extracted with EtOAc, dried over Na₂SO₄, and concentrated under vacuum. The crude product was purified by MPLC (SiO₂, 25 g, EtOAc in Hex 0-50%) to afford 5-cyclopropyl-3,8-dimethoxyphenanthridin-6(5H)-one (50 mg, 36%) as a brown solid. ¹H NMR (400 MHz, CDCl₃) δ 8.02 (dd, J=8.9, 2.1 Hz, 2H), 7.87 (d, J=2.8 Hz, 1H), 7.39 (d, J=2.5 Hz, 1H), 7.29 (dd, J=8.9, 2.8 Hz, 1H), 6.87 (dd, J=8.8, 2.5 Hz, 1H), 3.93 (d, J=3.2 Hz, 6H), 3.05-2.99 (m, 1H), 1.45-1.36 (m, 2H), 0.97-0.90 (m, 2H).

### Step 2: Synthesis of 5-cyclopropyl-3,8-dihydroxyphenanthridin-6(5H)-one

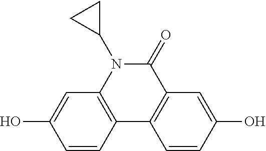 [SMILES: O=c1c2cc(O)ccc2c2ccc(O)cc2n1C1CC1]

5-cyclopropyl-3,8-dimethoxyphenanthridin-6(5H)-one (20 mg, 0.070 mmol, 1.0 eq.) was dissolved in DCM (1 mL) and cooled down to 0° C. in an ice-bath and stirring was continued for 5 min. Then, BBr₃ (0.20 ml, 1M in DCM, 0.020 mmol, 3.0 eq.) was added dropwise to the reaction mixture. Upon complete addition the mixture was left in the ice bath and was allowed to warm to r.t. over the course of 2 h. When no more starting material could be observed (TLC), the reaction mixture was dropwise added into 0° C. cold methanol (10 mL) and stirred for an additional 10 min. Then, the mixture was concentrated and loaded on silica to be purified by MPLC (SiO₂, 12 g, MeOH in DCM 0-5%) to afford 5-cyclopropyl-3,8-dihydroxyphenanthridin-6(5H)-one (13 mg, 0.050 mmol, 71%) as white solid. MS (ESI+): m/z=268. ¹H NMR (400 MHz, DMSO) δ 9.86 (d, J=9.3 Hz, 2H), 8.12 (d, J=8.9 Hz, 1H), 8.06 (d, J=8.8 Hz, 1H), 7.57 (d, J=2.7 Hz, 1H), 7.27 (d, J=2.3 Hz, 1H), 7.18 (dd, J=8.7, 2.8 Hz, 1H), 6.73 (dd, J=8.7, 2.3 Hz, 1H), 2.94 (dt, J=7.0, 3.1 Hz, 1H), 1.35-1.18 (m, 2H), 0.74 (p, J=5.4, 5.0 Hz, 2H).

### C) Sulfonamide “A” Group Analogues

### Synthesis of 3,8-dihydroxy-6H-dibenzo[c,e][1,2]thiazine 5,5-dioxide (22)

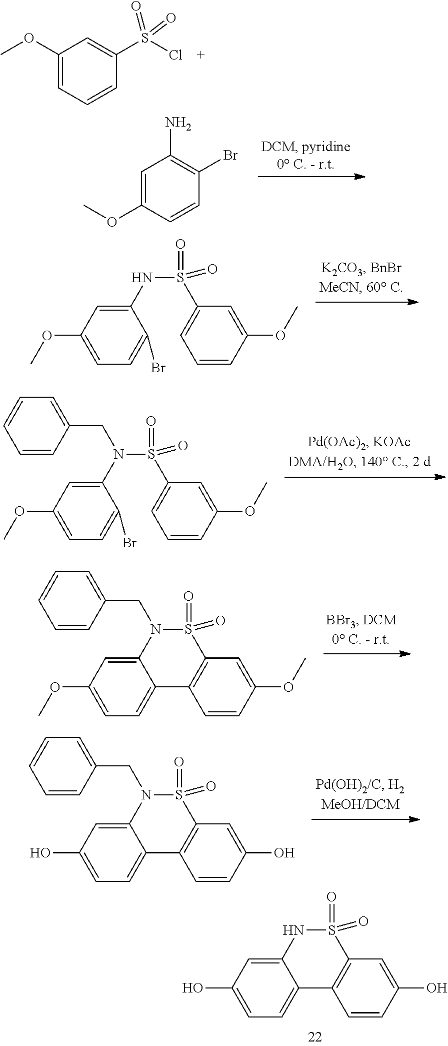

### Step 1: Synthesis of N-(2-bromo-5-methoxyphenyl)-3-methoxybenzenesulfonamide

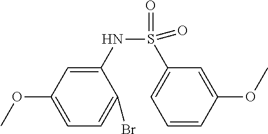

3-methoxybenzenesulfonyl chloride (2.00 g, 9.68 mmol, 1.3 eq.) was slowly added to a solution of 2-bromo-5-methoxyaniline (1.79 g, 8.81 mmol, 1.0 eq.) and pyridine (2.79 g, 35.2 mmol, 4.0 eq.) in DCM (20 mL) at 0° C. Upon warming up to r.t. no more starting material could be observed by TLC, and the reaction mixture was concentrated under vacuum. The reaction mixture was diluted with EtOAc and washed with 1N aqueous HCl. The organic phase was dried over Na₂SO₄ and concentrated in vacuum to afford N-(2-bromo-5-methoxyphenyl)-3-methoxybenzenesulfonamide (3.28 g, 99%) as a brown oil. ¹H NMR (400 MHz, CDCl₃) δ 7.41-7.21 (m, 4H), 7.07 (ddd, J=7.7, 2.5, 1.5 Hz, 1H), 6.94 (s, 1H), 6.55 (dd, J=8.9, 3.0 Hz, 1H), 3.78 (s, 3H), 3.77 (s, 3H).

### Step 2: Synthesis of N-benzyl-N-(2-bromo-5-methoxyphenyl)-3-methoxybenzenesulfonamide

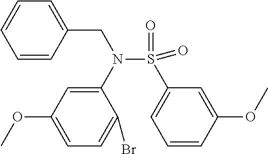

N-(2-bromo-5-methoxyphenyl)-3-methoxybenzenesulfonamide (5.90 g, 18.9 mmol, 1.0 eq.) was dissolved in MeCN (53 mL) and K₂CO₃ (6.57 g, 47.6 mmol, 3.0 eq.) was added in one portion. At r.t. benzyl bromide (2.98 g, 17.4 mmol, 1.1 eq.) was added dropwise and upon complete addition the reaction mixture was heated to 60° C. in an oil bath for 3 h. After complete consumption of the starting material (as indicated by TLC) the reaction mixture was allowed to cool down to r.t. and filtered. The filtrate was concentrated under vacuum and loaded on silica to be purified by MPLC (SiO₂, 240 g, EtOAc in Hex 0-10%) to afford N-benzyl-N-(2-bromo-5-methoxyphenyl)-3-methoxybenzenesulfonamide (6.83 g, 93%) as a light brown solid. ¹H NMR (400 MHz, CDCl₃) δ 7.42-7.34 (m, 3H), 7.28-7.18 (m, 6H), 7.15-7.10 (m, 1H), 6.69 (dd, J=8.9, 3.0 Hz, 1H), 6.48 (d, J=3.0 Hz, 1H), 4.89 (d, J=14.4 Hz, 1H), 4.66 (d, J=14.3 Hz, 1H), 3.79 (s, 3H), 3.59 (s, 3H).

### Step 3: Synthesis of 6-benzyl-3,8-dimethoxy-6H-dibenzo[c,e][1,2]thiazine 5,5-dioxide

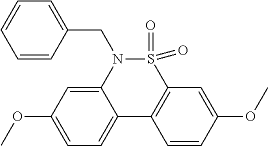

N-benzyl-N-(2-bromo-5-methoxyphenyl)-3-methoxybenzenesulfonamide (2.00 g, 4.33 mmol, 1.0 eq.) was dissolved in a mixture of DMA (20 mL) and water (5 mL) and thereupon were added Pd(OAc)₂ (291 mg, 1.30 mmol, 0.3 eq.) and KOAc (1.69 g, 17.3 mmol, 4.0 eq.). Upon complete dissolution of the reagents, the flask was put into a 140° C. oil bath and stirring was continued over a period of 48 h. Afterwards the reaction mixture was concentrated to complete dryness using a rotary evaporator at 90° C. The reaction mixture was loaded on silica and purified by MPLC (SiO₂, 80 g, EtOAc in Hex 0-15%) to afford 6-benzyl-3,8-dimethoxy-6H-dibenzo[c,e][1,2]thiazine 5,5-dioxide (560 mg, 34%) as a white solid. ¹H NMR (400 MHz, DMSO) δ 8.00 (t, J=8.5 Hz, 2H), 7.39 (d, J=2.7 Hz, 1H), 7.34 (dd, J=8.8, 2.7 Hz, 1H), 7.25-7.09 (m, 5H), 6.95 (d, J=2.5 Hz, 1H), 6.91 (dd, J=8.8, 2.5 Hz, 1H), 5.16 (s, 2H), 3.91 (s, 3H), 3.75 (s, 3H).

### Step 4: Synthesis of 6-benzyl-3,8-dihydroxy-6H-dibenzo[c,e][1,2]thiazine 5,5-dioxide

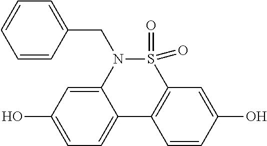 [SMILES: O=S1(=O)c2cc(O)ccc2-c2ccc(O)cc2N1Cc1ccccc1]

6-benzyl-3,8-dimethoxy-6H-dibenzo[c,e][1,2]thiazine 5,5-dioxide (180 mg, 0.470 mmol, 1.0 eq.) was dissolved in DCM (2 mL) and cooled down to 0° C. in an ice-bath and stirring was continued for 5 min. Then, BBr₃ (1.89 ml, 1M in DCM, 1.88 mmol, 4.0 eq.) was added dropwise to the reaction mixture. Upon complete addition the mixture was left in the ice bath and was allowed to warm to r.t. over the course of 2 h. When no more starting material could be observed (TLC), the reaction mixture was dropwise added into 0° C. cold methanol (20 mL) and stirred for an additional 10 min. Then, the mixture was concentrated, loaded on silica, and purified by MPLC (SiO₂, 20 g, MeOH in DCM 0-3%) to afford 6-benzyl-3,8-dihydroxy-6H-dibenzo[c,e][1,2]thiazine 5,5-dioxide (100 mg, 60%) as a light yellow solid. ¹H NMR (400 MHz, DMSO) δ 10.33 (s, 1H), 9.94 (s, 1H), 7.90-7.80 (m, 2H), 7.39-7.09 (m, 7H), 6.83-6.62 (m, 2H), 5.04 (s, 2H).

### Step 5: Synthesis of 3,8-dihydroxy-6H-dibenzo[c,e][1,2]thiazine 5,5-dioxide

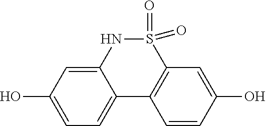 [SMILES: O=S1(=O)Nc2cc(O)ccc2-c2ccc(O)cc21]

6-benzyl-3,8-dihydroxy-6H-dibenzo[c,e][1,2]thiazine 5,5-dioxide (100 mg, 0.370 mmol, 1.0 eq.) was dissolved in MeOH (10 mL) and Pd(OH)₂/C (26 mg) was added in one portion. Then, the reaction mixture was evacuated and backfilled with N₂ three times before putting it under hydrogen atmosphere (balloon). The reaction mixture was stirred for 4 h and upon complete consumption of starting material (as indicated by TLC) filtered over silica and concentrated under vacuum. The crude product was loaded on silica, and purified by MPLC (SiO₂, 12 g, EtOAc in Hex 0-50%) to give 3,8-dihydroxy-6H-dibenzo[c,e][1,2]thiazine 5,5-dioxide (65 mg, 67%) as a white solid. ¹H NMR (400 MHz, DMSO) δ 10.24 (s, 1H), 9.87 (s, 1H), 7.88 (d, J=8.8 Hz, 1H), 7.83 (d, J=8.5 Hz, 1H), 7.17 (d, J=2.6 Hz, 1H), 7.11 (dd, J=8.7, 2.6 Hz, 1H), 6.64 (dd, J=8.7, 2.5 Hz, 1H), 6.55 (d, J=2.5 Hz, 1H).

### D) Ether “A” Group Analogues

### Synthesis of 6H-benzo[c]chromene-3,8-diol (23)

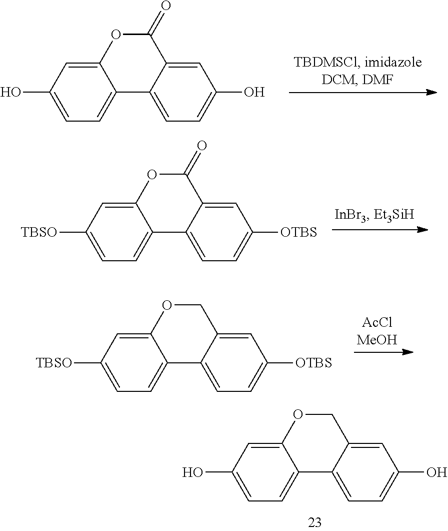

### Step 1: Synthesis of 3,8-bis((teri-butyldimethylsily)oxy)-6H-benzo[c]chromen-6-one

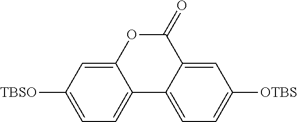 [SMILES: CC(C)(C)[Si](C)(C)Oc1ccc2c(c1)oc(=O)c1cc(O[Si](C)(C)C(C)(C)C)ccc12]

Urolithin A (12 g 53 mmol) was added to a solution of imidazole (9.0 g, 0.13 mol) in DCM (100 mL). stirred for 1 h. No reaction took place, therefore DMF (20 mL) was added and stirring continued overnight. DCM was removed in vacuum. Water was added and the mixture was extracted with Et₂O (3*), the organic layers were washed successively with water twice and brine, dried with Na₂SO₄, filtered over silica and concentrated. The crude product was purified by MPLC (SiO₂, EtOAc/Cyclohexane 0 to 20%) to afford 3,8-bis((tert-butyldimethylsilyl)oxy)-6H-benzo[c]chromen-6-one (20 g, 96%) as a white solid. ¹H NMR (400 MHz, CDCl₃) δ 7.89 (d, J=8.8 Hz, 1H), 7.85-7.80 (m, 1H), 7.76 (d, J=2.6 Hz, 1H), 7.29 (dd, J=8.7, 2.7 Hz, 1H), 6.86-6.80 (m, 2H), 1.02 (s, 9H), 0.98 (s, 9H), 0.26 (s, 6H), 0.24 (s, 6H).

### Step 2: Synthesis of ((6H-benzo[c]chromene-3,8-diyl)bis(oxy))bis(tert-butyldimethylsilane)

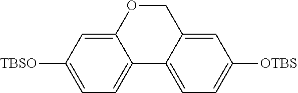 [SMILES: CC(C)(C)[Si](C)(C)Oc1ccc2c(c1)COc1cc(O[Si](C)(C)C(C)(C)C)ccc1-2]

InBr₃ (142 mg, 0.400 mmol) was added to a solution of 3,8-bis((tert-butyldimethylsilyl)oxy)-6H-benzo[c]chromen-6-one (1.8 g, 4.0 mmol) in toluene (20 ml) and the reaction mixture was heated at 70° C. for 1 hour. The reaction mixture was cooled down to room temperature and filtered. The solvent was evaporated under vacuum and the crude was purified by MPLC (SiO₂, cyclohexane/dichloromethane from 0% to 10%) to afford ((6H-benzo[c]chromene-3,8-diyl)bis(oxy))bis(tert-butyldimethylsilane), 81 mg, 88%) as a white solid. ¹H NMR (400 MHz, CDCl₃) δ 7.48 (t, J=8.5 Hz, 2H), 6.81 (dd, J=8.4, 2.5 Hz, 1H), 6.60 (d, J=2.4 Hz, 1H), 6.53 (dd, J=8.4, 2.5 Hz, 1H), 6.47 (d, J=2.4 Hz, 1H), 5.02 (s, 2H), 1.00 (s, 9H), 0.98 (s, 9H), 0.22 (s, 6H), 0.20 (s, 6H).

### Step 3: Synthesis of 6H-benzo[c]chromene-3,8-diol

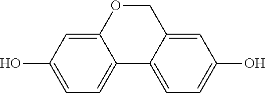 [SMILES: Oc1ccc2c(c1)COc1cc(O)ccc1-2]

Acetyl chloride (0.105 ml, 1.40 mmol) was added to a solution of ((6H-benzo[c]chromene-3,8-diyl)bis(oxy))bis(tert-butyldimethylsilane) (421 mg, 0.950 mmol) in methanol (10 ml) at room temperature and stirred overnight. The reaction mixture was concentrated under vacuum and purified by MPLC (SiO₂, EtOAc in Hex 0-100%) to afford 6H-benzo[c]chromene-3,8-diol (203 mg, 0.950 mmol, 99%) as a white solid. ¹H NMR (400 MHz, DMSO) δ 9.50 (s, 1H), 9.48 (s, 1H), 7.49 (dd, J=12.5, 8.4 Hz, 2H), 6.74 (dd, J=8.4, 2.6 Hz, 1H), 6.60 (d, J=2.5 Hz, 1H), 6.45 (dd, J=8.4, 2.4 Hz, 1H), 6.32 (d, J=2.4 Hz, 1H), 4.96 (s, 2H).

### Synthesis of 6-methyl-6H-benzo[c]chromene-3,8-diol (24)

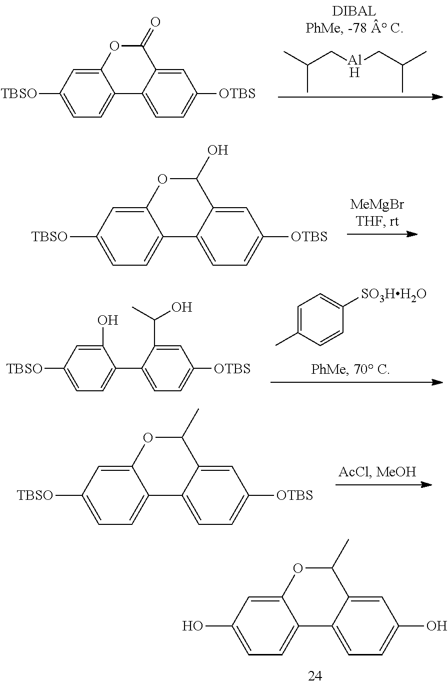

### Step 1: Synthesis of 3,8-bis((tert-butyldimethylsilyl)oxy)-6H-benzo[c]chromen-6-ol

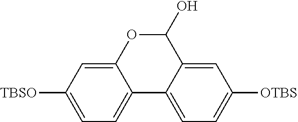 [SMILES: CC(C)(C)[Si](C)(C)Oc1ccc2c(c1)OC(O)c1cc(O[Si](C)(C)C(C)(C)C)ccc1-2]

DIBAL-H (2.10 mL, 2.10 mmol) was added slowly along the side of the flask to a solution of 3,8-bis((tert-butyldimethylsilyl)oxy)-6H-benzo[c]chromen-6-one (912 mg, 2.00 mmol) in toluene (20 ml) under nitrogen at −78° C. The reaction was monitored by TLC eluent (Cyclohexane/DCM 1:1). The reaction was complete within 1 hour stirring. After a Fieser work-up the product was used in the step without further purification. ¹H NMR (400 MHz, CDCl₃) δ 7.60 (dd, J=8.9, 6.9 Hz, 2H), 6.93 (dd, J=8.5, 2.5 Hz, 1H), 6.83 (d, J=2.6 Hz, 1H), 6.62-6.58 (m, 2H), 6.26 (s, 1H), 1.00 (s, 9H), 0.98 (s, 9H), 0.25-0.18 (m, 12H).

### Step 2: Synthesis of 4,4′-bis((tert-butyldimethylsilyl)oxy)-2′-(1-hydroxyethyl)-[1,1′-biphenyl]-2-ol

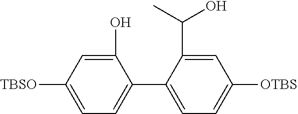 [SMILES: CC(O)c1cc(O[Si](C)(C)C(C)(C)C)ccc1-c1ccc(O[Si](C)(C)C(C)(C)C)cc1O]

To a solution of 3,8-bis((tert-butyldimethylsilyl)oxy)-6H-benzo[c]chromen-6-ol (456 mg, 1.00 mmol, 1.0 eq.) in anhydrous THF (10 mL) was slowly added at 0° C. MeMgr (3M in Et₂O, 1.0 mL, 3.0 mmol, 3.0 eq.) under a nitrogen atmosphere. The reaction was complete within 1 hour. The reaction mixture was diluted with ether, filtered over a pad of silica, with ether washings, and concentrated to afford the title product as a thick colourless oily 60:40 mixture of rota/diastereomers (474 mg, quant.), which was used in the next step without further purification. ¹H NMR (400 MHz, CDCl₃) δ 7.15 (d, J=2.6 Hz, 0.4H), 7.12 (d, J=2.6 Hz, 0.6H), 7.07 (s, 0.4H), 7.04 (s, 0.6H), 6.96 (d, J=8.1 Hz, 0.4H), 6.90 (d, J=8.5 Hz, 0.6H), 6.86-6.78 (m, 1H), 6.52-6.43 (m, 2H), 4.79 (q, J=6.4 Hz, 0.4H), 4.73 (q, J=6.5 Hz, 0.6H), 1.36 (d, J=6.4 Hz, 1.2H), 1.30 (d, J=6.4 Hz, 1.8H), 1.01 (s, 7.2H), 1.00 (s, 10.8H), 0.25 (s, 4.8H), 0.24 (s, 7.2H).

### Step 3: ((6-methyl-6H-benzo[c]chromene-3,8-diyl)bis(oxy))bis(tert-butyldimethylsilane)

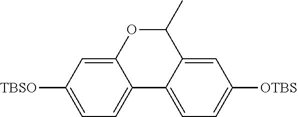 [SMILES: CC1Oc2cc(O[Si](C)(C)C(C)(C)C)ccc2-c2ccc(O[Si](C)(C)C(C)(C)C)cc21]

A solution of 4-methylbenzenesulfonic acid hydrate (19 mg, 0.19 mmol) and 4,4′-bis((tert-butyldimethylsilyl)oxy)-2′-(1-hydroxyethyl)-[1,1′-biphenyl]-2-ol (474 mg, 1.00 mmol) in toluene (10 mL) was heated at 80° C. overnight. TLC (Cyclohexane/dichlorometahne 9:1) showed no more starting material. The reaction mixture was concentrated under vacuum and purified by column (SiO₂, CyH/DCM) to afford ((6-methyl-6H-benzo[c]chromene-3,8-diyl)bis(oxy))bis(tert-butyldimethylsilane) (411 mg, 90%) as a white solid. ¹H NMR (400 MHz, CDCl₃) δ 7.48 (dd, J=8.5, 4.1 Hz, 2H), 6.80 (dd, J=8.4, 2.5 Hz, 1H), 6.61 (dd, J=2.4, 0.8 Hz, 1H), 6.52 (dd, J=8.4, 2.4 Hz, 1H), 6.47 (d, J=2.4 Hz, 1H), 5.17 (q, J=6.5 Hz, 1H), 1.00 (s, 9H), 0.98 (s, 9H) 0.92-0.84 (m, 3H), 0.21 (s, 6H), 0.20 (s, 6H).

### Step 4: 6-methyl-6H-benzo[c]chromene-3,8-diol

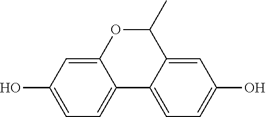 [SMILES: CC1Oc2cc(O)ccc2-c2ccc(O)cc21]

Acetylchloride (0.100 ml, 1.40 mmol) was added to a solution of ((6H-benzo[c]chromene-3,8-diyl)bis(oxy))bis(tert-butyldimethylsilane) (411 mg, 0.900 mmol) in methanol (10 ml) at room temperature and stirred overnight. The reaction mixture was concentrated under vacuum and purified by MPLC (SiO₂, EtOAc in Hex 0-100%) to afford the 6-methyl-6H-benzo[c]chromene-3,8-diol (202 mg, 98%) as a white solid. ¹H NMR (400 MHz, DMSO) δ 9.47 (s, 1H), 9.45 (s, 1H), 7.49 (t, J=8.7 Hz, 2H), 6.73 (dd, J=8.4, 2.5 Hz, 1H), 6.60 (d, J=2.4 Hz, 1H), 6.43 (dd, J=8.4, 2.4 Hz, 1H), 6.30 (d, J=2.4 Hz, 1H), 5.14 (q, J=6.5 Hz, 1H), 1.44 (d, J=6.5 Hz, 3H).

### Synthesis of 6,6-dimethyl-6H-benzo[c]chromene-3,8-diol (25)

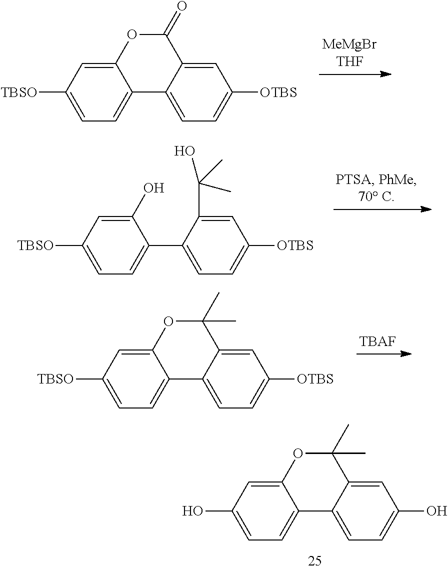

### Step 1: Synthesis of 4,4′-bis((tert-butyldimethylsilyl)oxy)-2′-(2-hydroxypropan-2-yl)-[1,1′-biphenyl]-2-ol

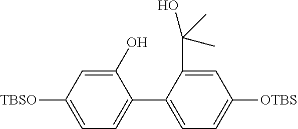 [SMILES: CC(C)(O)c1cc(O[Si](C)(C)C(C)(C)C)ccc1-c1ccc(O[Si](C)(C)C(C)(C)C)cc1O]

To a solution of 3,8-bis((tert-butyldimethylsilyl)oxy)-6H-benzo[c]chromen-6-one (456 mg, 1.00 mmol, 1.0 eq.) in anhydrous THF (10 mL) was slowly added at 0° C. MeMgBr (3M in Et₂O, 1.00 mL, 3.00 mmol, 3.0 eq.) under a nitrogen atmosphere. The reaction was complete within 1 hour. The reaction mixture was diluted with ether, filtered over a pad of silica, with ether washings, and concentrated to afford the synthesis of 4,4′-bis((tert-butyldimethylsilyl)oxy)-2′-(2-hydroxypropan-2-yl)-[1,1′-biphenyl]-2-ol as a thick colourless oil (489 mg, quant.), which was used in the next step without further purification. ¹H NMR (400 MHz, CDCl₃) δ 7.13 (d, J=2.5 Hz, 1H), 6.96 (d, J=1.0 Hz, 1H), 6.94 (d, J=1.1 Hz, 1H), 6.76 (dd, J=8.2, 2.6 Hz, 1H), 6.49 (d, J=2.4 Hz, 1H), 6.45 (dd, J=8.2, 2.4 Hz, 1H), 1.52 (s, 3H), 1.40 (s, 3H), 1.01 (s, 9H), 1.00 (s, 9H), 0.25 (s, 6H), 0.23 (s, 6H).

### Step 2: Synthesis of ((6,6-dimethyl-6H-benzo[c]chromene-3,8-diyl)bis(oxy))bis(tert-butyldimethylsilane)

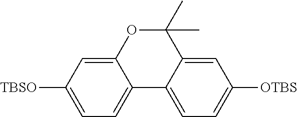 [SMILES: CC1(C)Oc2cc(O[Si](C)(C)C(C)(C)C)ccc2-c2ccc(O[Si](C)(C)C(C)(C)C)cc21]

A solution of 4-methylbenzenesulfonic acid hydrate (19 mg, 0.19 mmol) and 4,4′-bis((tert-butyldimethylsilyl)oxy)-2′-(1-hydroxyethyl)-[1,1′-biphenyl]-2-ol (489 mg, 1.00 mmol) in toluene (10 ml) was heated at 80° C. overnight. TLC (Cyclohexane/dichloromethane 9:1) showed no more starting material. The reaction mixture was concentrated under vacuum and purified by MPLC (SiO₂, CyH/DCM) to afford ((6-methyl-6H-benzo[c]chromene-3,8-diyl)bis(oxy))bis(tert-butyldimethylsilane) (446 mg, 95%) as a white solid. ¹H NMR (400 MHz, CDCl₃) δ 7.49 (dd, J=8.5, 3.4 Hz, 2H), 6.79 (dd, J=8.4, 2.4 Hz, 1H), 6.69 (d, J=2.4 Hz, 1H), 6.50 (dd, J=8.4, 2.4 Hz, 1H), 6.45 (d, J=2.4 Hz, 1H), 1.58 (s, 6H), 1.00 (s, 9H), 0.99 (s, 9H), 0.22 (s, 6H), 0.21 (s, 6H).

### Step 3: Synthesis 6,6-dimethyl-6H-benzo[c]chromene-3,8-diol

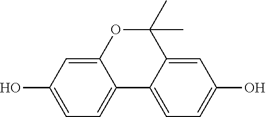 [SMILES: CC1(C)Oc2cc(O)ccc2-c2ccc(O)cc21]

Acetyl chloride (0.100 mL, 1.40 mmol) was added to a solution of 4,4′-bis((tert-butyldimethylsilyl)oxy)-2′-(2-hydroxypropan-2-yl)-[1,1′-biphenyl]-2-ol (446 mg, 0.900 mmol) in methanol (10 ml) at room temperature and the solution was stirred overnight. The reaction mixture was concentrated under vacuum and the residue purified by MPLC (SiO₂, EtOAc in Hex 0-100%) to afford the 6,6-dimethyl-6H-benzo[c]chromene-3,8-diol (228 mg, 98%) as a white solid. MS (ESI+): m/z=243. ¹H NMR (400 MHz, DMSO) δ 9.44 (s, 1H), 9.42 (s, 1H), 7.49 (dd, J=8.5, 4.2 Hz, 2H), 6.72 (dd, J=8.4, 2.5 Hz, 1H), 6.67 (d, J=2.4 Hz, 1H), 6.40 (dd, J=8.4, 2.4 Hz, 1H), 6.26 (d, J=2.4 Hz, 1H), 1.49 (s, 6H).

### E) Ester “A” Group Analogues with Pyridine Ring

### Synthesis of 3,8-dihydroxy-6H-isochromeno[4,3-b]pyridin-6-one (27)

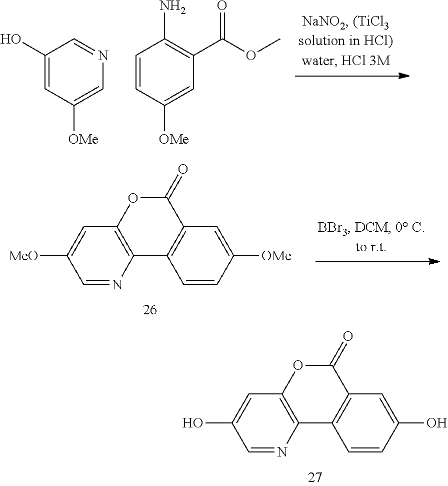

### Step 1: Synthesis of 3,8-dimethoxy-6H-isochromeno[4,3-b]pyridin-6-one (26)

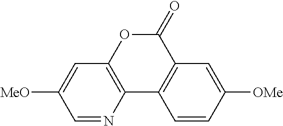 [SMILES: COc1cnc2c(c1)oc(=O)c1cc(OC)ccc12]

Sodium nitrite (130 mg, 1.88 mmol) was added to a solution of methyl 2-amino-5-methoxybenzoate (341 mg, 1.88 mmol) in water (1 mL) and HCl (3N, 1 mL) at 0° C. The reaction mixture was stirred 15 min at 0° C., and this solution was added dropwise to a solution of 5-methoxypyridin-3-ol (1.18 g, 9.42 mmol) in water (1 mL) and HCl (3N, 1 mL) and TiCl₃ (0.25 ml, 1.88 mmol) at 0° C., and stirring continued o.n. at rt. A saturated solution of Na₂CO₃ was added. After extraction with EtOAc 3 times the combined organic phases were dried over sodium sulfate and concentrated under vacuum. The crude product was purified by MPLC (SiO₂, EtOAc/cyclohexane 0% to 30%) to afford 3,8-dimethoxy-6H-isochromeno[4,3-b]pyridin-6-one (85 mg, 18%) as a white solid. R_{f}=0.25 (EtOAc/hexane 20%). ¹H NMR (400 MHz, CDCl₃) δ 8.47 (d, J=8.8 Hz, 1H), 8.33 (d, J=2.6 Hz, 1H), 7.74 (d, J=2.7 Hz, 1H), 7.45 (dd, J=8.8, 2.7 Hz, 1H), 7.14 (d, J=2.6 Hz, 1H), 3.95 (s, 3H), 3.93 (s, 3H).

### Step 2: Synthesis of 3,8-dihydroxy-6H-isochromeno[4,3-b]pyridin-6-one (27)

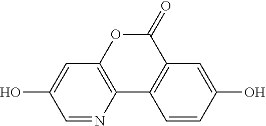 [SMILES: O=c1oc2cc(O)cnc2c2ccc(O)cc12]

27 was prepared according to GP2 starting from 3,8-dimethoxy-6H-isochromeno[4,3-b]pyridin-6-one 26 (120 mg, 0.460 mmol) to afford after purification by MPLC (SiO₂, EtOAc/cyclohexane 5% to 90%) 3,8-dihydroxy-6H-isochromeno[4,3-b]pyridin-6-one (20 mg, 19%) as a white solid. R_{f}=0.1 (EtOAc/hexane 80%). ¹H NMR (400 MHz, DMSO) δ 10.60 (s, 1H), 10.39 (s, 1H), 8.30 (d, J=8.7 Hz, 1H), 8.19 (d, J=2.4 Hz, 1H), 7.50 (d, J=2.6 Hz, 1H), 7.38 (dd, J=8.7, 2.6 Hz, 1H), 7.15 (d, J=2.4 Hz, 1H).

### Synthesis of 3,8-dihydroxy-5H-chromeno[4,3-b]pyridin-5-one (28)

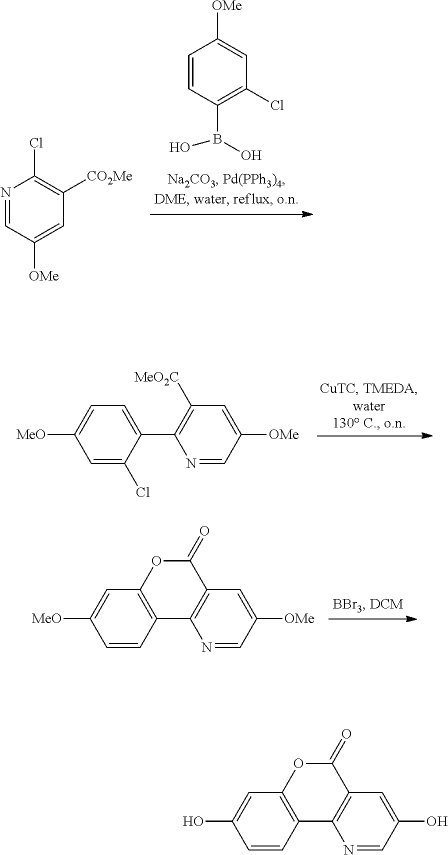 [SMILES: COC(=O)c1cc(OC)cnc1Cl ; COc1ccc(B(O)O)c(Cl)c1 ; COC(=O)c1cc(OC)cnc1-c1ccc(OC)cc1Cl ; COc1ccc2c(c1)oc(=O)c1cc(OC)cnc12 ; O=c1oc2cc(O)ccc2c2ncc(O)cc12]

### Step 1: Synthesis of methyl 2-(2-chloro-4-methoxyphenyl)-5-methoxynicotinate

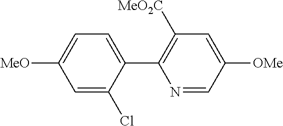 [SMILES: COC(=O)c1cc(OC)cnc1-c1ccc(OC)cc1Cl]

Water (1 ml) was added to a mixture of (2-hydroxy-4-methoxyphenyl)boronic acid (144 mg, 0.774 mmol), methyl 2-chloro-5-methoxynicotinate (120 mg, 0.595 mmol), cesium carbonate (170 mg, 1.61 mmol) and palladium tetrakis(triphenylphosphine)palladium (35 mg, 0.029 mmol) in DME (5 ml) and the mixture was refluxed for 3 h. TLC showed complete conversion of the starting material. A saturated solution of NH₄Cl was added and the aqueous phase was extracted with EtOAc 3 times. The combined organic phases were dried over sodium sulfate and concentrated under vacuum. The crude product was purified by MPLC (SiO₂, EtOAc/hexane 0% to 60%) to afford methyl 2-(2-chloro-4-methoxyphenyl)-5-methoxynicotinate (160 mg, 87%) as a colorless oil. R_{f}=0.3 (EtOAc/hexane 50%). ¹H NMR (400 MHz, CDCl₃) δ 8.49 (d, J=3.0 Hz, 1H), 7.77 (d, J=3.0 Hz, 1H), 7.33 (d, J=8.5 Hz, 1H), 6.96 (d, J=2.5 Hz, 1H), 6.90 (dd, J=8.5, 2.5 Hz, 1H), 3.95 (s, 3H), 3.84 (s, 3H), 3.74 (s, 3H).

### Step 2: Synthesis of 3,8-dimethoxy-5H-chromeno[4,3-b]pyridin-5-one

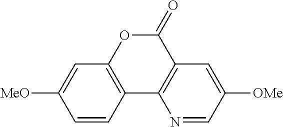 [SMILES: COc1ccc2c(c1)oc(=O)c1cc(OC)cnc12]

In a microwave vessel, to a mixture of methyl 2-(2-chloro-4-methoxyphenyl)-5-methoxynicotinate (900 mg, 2.92 mmol, 1.0 eq.), Copper(I) thiophene-2-carboxylate (278 mg, 1.46 mmol, 0.5 eq.), Cs₂CO₃ (476 mg, 1.46 mmol, 0.5 eq.) in deionized water (10 mL) was added TMEDA (339 mg, 2.92 mmol, 1.0 eq.) via micro-syringe. The mixture was allowed to stir at room temperature for 15 min and then refluxed at 130° C. overnight. The reaction mixture was cooled down to r.t. and extracted with EtOAc and with a saturated solution of NH₄Cl. The organic phases were dried over sodium sulfate and concentrated under vacuum. The crude product was purified by MPLC (SiO₂, EtOAc/cyclohexane 0% to 30%) to afford 3,8-dimethoxy-5H-chromeno[4,3-b]pyridin-5-one (120 mg, 16%) as a white solid. R_{f}=0.4 (EtOAc/hexane 80%). ¹H NMR (400 MHz, CDCl₃) δ 8.69 (d, J=3.1 Hz, 1H), 8.37 (d, J=8.8 Hz, 1H), 7.93 (d, J=3.1 Hz, 1H), 6.96 (dd, J=8.8, 2.5 Hz, 1H), 6.87 (d, J=2.4 Hz, 1H), 3.96 (s, 3H), 3.89 (s, 3H).

### Step 3: Synthesis of 3,8-dihydroxy-5H-chromeno[4,3-b]pyridin-5-one

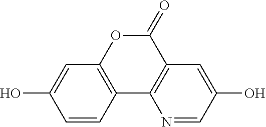 [SMILES: O=c1oc2cc(O)ccc2c2ncc(O)cc12]

28 was prepared according to GP2 starting from 3,8-dimethoxy-5H-chromeno[4,3-b]pyridin-5-one (120 mg, 0.460 mmol) to afford after purification by MPLC (SiO₂, MeOH/DCM 0% to 10%) 3,8-dihydroxy-5H-chromeno[4,3-b]pyridin-5-one (26 mg, 56%) as a white solid. R_{f}=0.1 (EtOAc/hexane 80%). ¹H NMR (400 MHz, DMSO) δ 10.55 (s, 1H), 10.50 (s, 1H), 8.62 (d, J=2.9 Hz, 1H), 8.19 (dd, J=8.6, 1.5 Hz, 1H), 7.74 (d, J=2.9 Hz, 1H), 6.86 (dd, J=8.7, 2.3 Hz, 1H), 6.76 (d, J=2.3 Hz, 1H).

### F) Ester “A” Ring Analogues with Ether Substitution Prepared by Mitsunobu Reaction

The Mitsunobu targets were achieved starting from two common intermediates (CI1 and CI2) which are described below.

### Synthesis of CI1

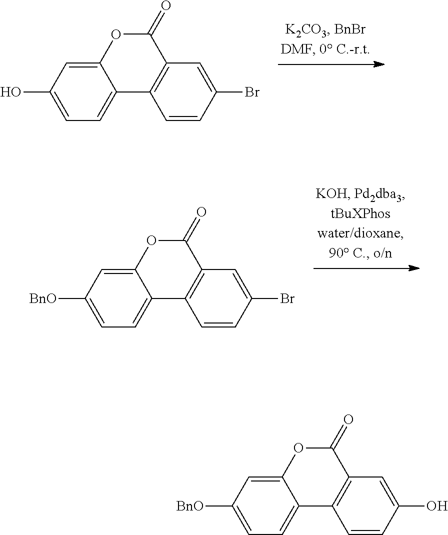 [SMILES: O=c1oc2cc(O)ccc2c2ccc(Br)cc12 ; O=c1oc2cc(OCc3ccccc3)ccc2c2ccc(Br)cc12 ; O=c1oc2cc(OCc3ccccc3)ccc2c2ccc(O)cc12]

### Step 1: Synthesis of 3-(benzyloxy)-8-bromo-6H-benzo[c]chromen-6-one

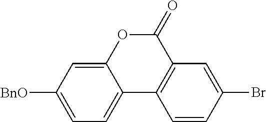 [SMILES: O=c1oc2cc(OCc3ccccc3)ccc2c2ccc(Br)cc12]

To a suspension of 3 (synthesis vide supra) (500 mg, 1.72 mmol, 1.0 eq.) in DMF (5 mL) was added in one portion K₂CO₃ (522 mg, 3.78 mmol, 2.2 eq.). Following the suspension was cooled to 0° C. in an ice-bath and stirred for 5 min. Benzyl bromide (323 mg, 1.89 mmol, 1.2 eq.) was added dropwise over a period of 1 min and upon complete addition the reaction mixture was stirred at 0° C. for 10 min before being allowed to warm up to room temperature overnight. After the complete consumption of starting material (as indicated by TLC) the reaction mixture was quenched with half-saturated aqueous sodium bicarbonate solution. The precipitate was filtered over a Buchner funnel, washed with hexanes and dried to obtain 3-(benzyloxy)-8-bromo-6H-benzo[c]chromen-6-one (400 mg, 61%) as a light brown solid. ¹H NMR (400 MHz, CDCl3) δ 10.27 (s, 1H), 8.18 (d, J=8.8 Hz, 1H), 8.14 (d, J=8.9 Hz, 1H), 7.53 (d, J=2.7 Hz, 1H), 7.51-7.47 (m, 2H), 7.44-7.39 (m, 2H), 7.37-7.32 (m, 1H), 7.07 (d, J=2.5 Hz, 1H), 7.04 (dd, J=8.7, 2.5 Hz, 1H), 5.21 (s, 2H).

### Step 2: Synthesis of 3-(benzyloxy)-8-hydroxy-6H-benzo[c]chromen-6-one CI1

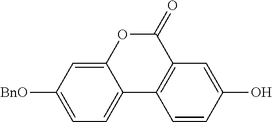 [SMILES: O=c1oc2cc(OCc3ccccc3)ccc2c2ccc(O)cc12]

3-(benzyloxy)-8-bromo-6H-benzo[c]chromen-6-one (700 mg, 1.84 mmol, 1.0 eq.) was suspended in 1,4-dioxane (7 mL) in a 20 mL Biotage MW vial. To this suspension was added Pd₂dba₃ (43 mg, 0.18 mmol, 0.1 eq.) followed by ₜBuXPhos (175 mg, 0.370 mmol, 0.2 eq.). Subsequently the MW vial was sealed and degassed with nitrogen for 10 min. Then, a solution of KOH (412 mg, 7.34 mmol, 4.4 eq.) in H₂O (3 mL) was added slowly to the reaction mixture, which was then stirred in a pre-heated oil bath at 90° C. for 3 h. Upon complete consumption of starting material (as indicated by TLC) the reaction mixture was cooled to 0° C. and the pH was adjusted to 1 with 6M aqueous HCl. The mixture was extracted with ethyl acetate (3×10 mL) and the combined organic phases were dried over anhydrous Na₂SO₄ and concentrated under vacuum. The crude material was purified by MPLC (SiO₂, 40 g, EtOAc in Hexanes 0-30%) to afford 3-(benzyloxy)-8-hydroxy-6H-benzo[c]chromen-6-one (390 mg, 67%) as a light yellow solid. ¹H NMR (400 MHz, DMSO) δ 10.27 (s, 1H), 8.18 (d, J=8.8 Hz, 1H), 8.14 (d, J=8.9 Hz, 1H), 7.53 (d, J=2.7 Hz, 1H), 7.51-7.47 (m, 2H), 7.44-7.39 (m, 2H), 7.37-7.32 (m, 2H), 7.07 (d, J=2.5 Hz, 1H), 7.04 (dd, J=8.7, 2.5 Hz, 1H), 5.21 (s, 2H).

### Synthesis of CI2

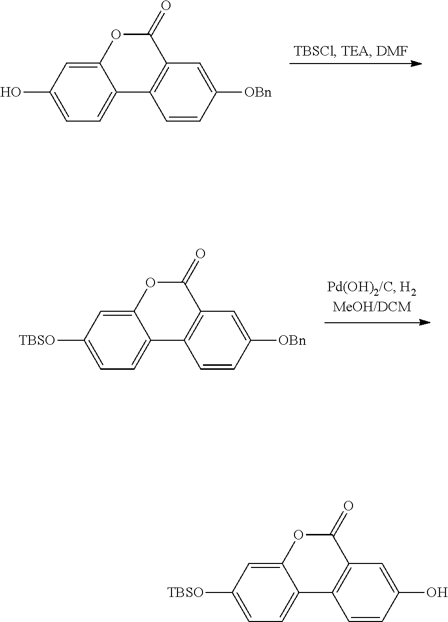 [SMILES: O=c1oc2cc(O)ccc2c2ccc(OCc3ccccc3)cc12 ; CC(C)(C)[Si](C)(C)Oc1ccc2c(c1)oc(=O)c1cc(OCc3ccccc3)ccc12 ; CC(C)(C)[Si](C)(C)Oc1ccc2c(c1)oc(=O)c1cc(O)ccc12]

### Step 1: Synthesis of 8-(benzyloxy)-3-((tert-butyldimethylsilyl)oxy)-6H-benzo[c]chromen-6-one

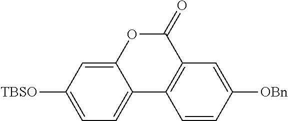 [SMILES: CC(C)(C)[Si](C)(C)Oc1ccc2c(c1)oc(=O)c1cc(OCc3ccccc3)ccc12]

8-(benzyloxy)-3-hydroxy-6H-benzo[c]chromen-6-one (1.46 g, 4.59 mmol) was dissolved in dry THF 12 ml. Triethylamine (1.92 ml, 13.8 mmol) was added dropwise at room temperature and stirred for 15 minutes then tert-butylchlorodimethylsilane (832 mg, 5.51 mmol) was added and stirring continued for 3 hours at room temperature. TLC showed no more starting material. The reaction mixture was extracted with EtOAc and HCl (1M) twice. The organic phases were washed successively with water and brine then dried over sodium sulfate to afford 8-(benzyloxy)-3-((tert-butyldimethylsilyl)oxy)-6H-benzo[c]chromen-6-one (1.83 g, 92%) as a brownish solid. ¹H NMR (400 MHz, CDCl₃) δ 7.94 (d, J=8.9 Hz, 1H), 7.88 (d, J=2.8 Hz, 1H), 7.86-7.81 (m, 1H), 7.50-7.34 (m, 6H), 6.86-6.80 (m, 2H), 5.18 (s, 2H), 1.00 (s, 9H), 0.25 (s, 6H).

### Step 2: Synthesis of 3-((tert-butyldimethylsilyl)oxy)-8-hydroxy-6H-benzo[c]chromen-6-one C2

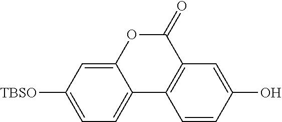 [SMILES: CC(C)(C)[Si](C)(C)Oc1ccc2c(c1)oc(=O)c1cc(O)ccc12]

8-(benzyloxy)-3-((tert-butyldimethylsilyl)oxy)-6H-benzo[c]chromen-6-one (1.83 g, 4.23 mmol, 1.0 eq) was dissolved in Methanol (20 ml) and dichloromethane (10 ml), Pd(OH)₂/C (368 mg, 0.5 mmol, 0.12 eq) was added and the reaction mixture was hydrogenated under atmospheric pressure o.n. The mixture was filtered over a pad of celite and the solvent evaporated under vacuum to afford 3-((tert-butyldimethylsilyl)oxy)-8-hydroxy-6H-benzo[c]chromen-6-one (1.3 g, 3.8 mmol, 90%) as a beige solid. ¹H NMR (400 MHz, CDCl₃) δ 7.95 (d, J=8.8 Hz, 1H), 7.91 (d, J=2.7 Hz, 1H), 7.89-7.83 (m, 1H), 7.39 (dd, J=8.7, 2.8 Hz, 1H), 6.92-6.83 (m, 2H), 6.21 (s, 1H), 1.03 (s, 9H), 0.28 (s, 6H).

### Synthesis of 3-hydroxy-8-(oxetan-3-ylmethoxy)-6H-benzo[c]chromen-6-one (29)

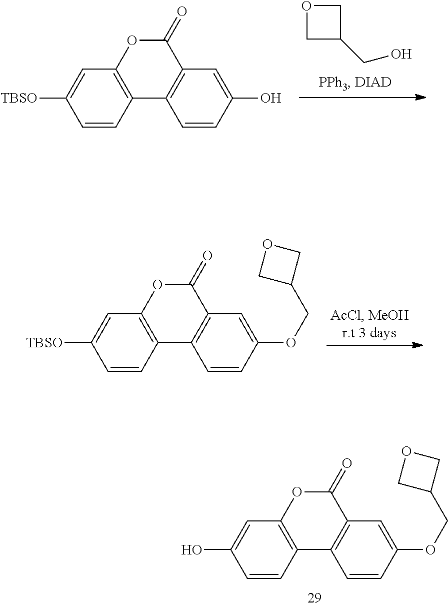 [SMILES: CC(C)(C)[Si](C)(C)Oc1ccc2c(c1)oc(=O)c1cc(O)ccc12 ; OCC1COC1 ; CC(C)(C)[Si](C)(C)Oc1ccc2c(c1)oc(=O)c1cc(OCC3COC3)ccc12 ; O=c1oc2cc(O)ccc2c2ccc(OCC3COC3)cc12]

### Step 1: Synthesis of 3-((tert-butyldimethylsilyl)oxy)-8-(oxetan-3-ylmethoxy)-6H-benzo[c]chromen-6-one

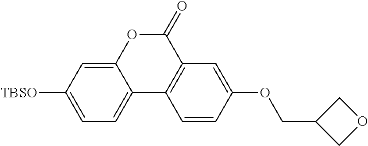 [SMILES: CC(C)(C)[Si](C)(C)Oc1ccc2c(c1)oc(=O)c1cc(OCC3COC3)ccc12]

In a sealed tube, DIAD (0.187 ml, 0.960 mmol) was added to a solution of a solution of 3-((tert-butyldimethylsilyl)oxy)-8-hydroxy-6H-benzo[c]chromen-6-one (150 mg, 0.430 mmol) and oxetan-3-ylmethanol (58 mg, 0.65 mmol) in THF (2 mL) at 0° C. and stirring continued overnight at room temperature. TLC indicated complete conversion of the starting material. The reaction mixture was loaded on silica gel and purified by MPLC (SiO₂, EtOAc/cyclohexane 0% to 30%) to afford 280 mg of a mixture of 3-((tert-butyldimethylsilyl)oxy)-8-(oxetan-3-ylmethoxy)-6H-benzo[c]chromen-6-one and reduced DIAD. R_{f}=0.3 (EtOAc/hexane 20/80). After purification there was a significant amount of reduced DIAD present in the NMR therefore it is not further described since it was used crude in the next step.

### Step 2: Synthesis of 3-hydroxy-8-(oxetan-3-ylmethoxy)-6H-benzo[c]chromen-6-one

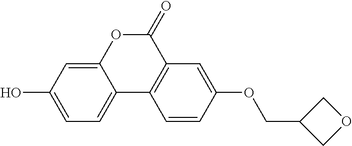 [SMILES: O=c1oc2cc(O)ccc2c2ccc(OCC3COC3)cc12]

KHF₂ (108 mg, 1.38 mmol) was added in one portion at r.t. to a solution of 3-((tert-butyldimethylsilyl)oxy)-8-(oxetan-3-ylmethoxy)-6H-benzo[c]chromen-6-one (285 mg, 0.690 mmol) (crude mixture of PPh₃0 and reduced DIAD) in MeOH (5 ml) and was stirred for 4 h. The formed white precipitate was filtered and dried under vacuum to afford 3-hydroxy-8-(oxetan-3-ylmethoxy)-6H-benzo[c]chromen-6-one (65 mg, 32%) as a white solid. ¹H NMR (400 MHz, DMSO) δ 10.27 (s, 1H), 8.26-8.06 (m, 2H), 7.64-7.52 (m, 1H), 7.52-7.27 (m, 1H), 7.08-6.96 (m, 1H), 6.85-6.71 (m, 1H), 4.73 (ddd, J=7.6, 6.0, 1.4 Hz, 2H), 4.46 (dt, J=11.9, 6.1 Hz, 2H), 4.33 (dd, J=18.2, 6.7 Hz, 2H), 3.43 (tt, J=6.8, 6.8 Hz, 1H).

### Synthesis of 3-hydroxy-8-(2-(4-methylpiperazin-1-yl)ethoxy)-6H-benzo[c]chromen-6-one (30)

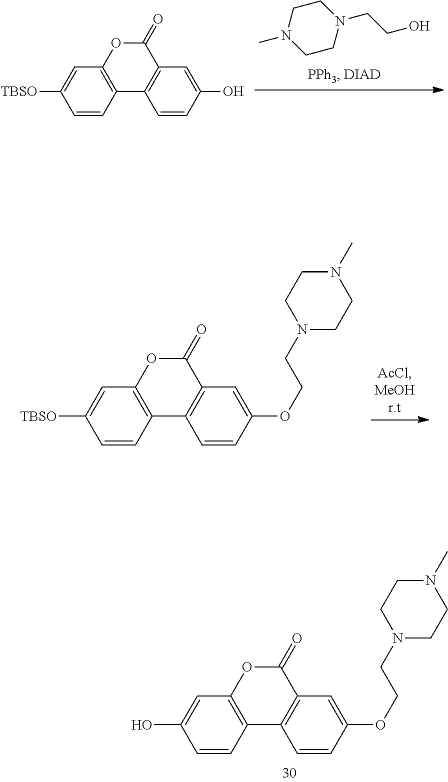

### Step 1: Synthesis of 3-((tert-butyldimethylsilyl)oxy)-8-(2-(4-methylpiperazin-1-yl)ethoxy)-6H-benzo[c]chromen-6-one)

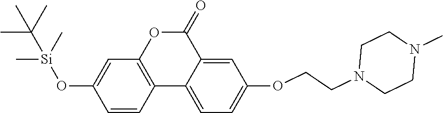 [SMILES: CN1CCN(CCOc2ccc3c(c2)c(=O)oc2cc(O[Si](C)(C)C(C)(C)C)ccc23)CC1]

3-((tert-butyldimethylsilyl)oxy)-8-(2-(4-methylpiperazin-1-yl)ethoxy)-6H-benzo[c]chromen-6-one was prepared from 3-((tert-butyldimethylsilyl)oxy)-8-hydroxy-6H-benzo[c]chromen-6-one (80 mg, 0.23 mmol) and 2-(4-Methyl-piperazin-1-yl)-ethanol (34 mg, 0.23 mmol) (according to the synthesis of 29) to afford after MPLC purification (SiO₂, MeOH/DCM 0% to 20%) 3-((tert-butyldimethylsilyl)oxy)-8-(2-(4-methylpiperazin-1-yl)ethoxy)-6H-benzo[c]chromen-6-one (60 mg, 55%) as yellowish oil. The NMR still showed significant amounts of reduced DIAD, but the impure/crude material was carried forward to the next step. R_{f}=0.4 (20% MeOH/DCM).

### Step 2: Synthesis of 3-hydroxy-8-(2-(4-methylpiperazin-1-yl)ethoxy)-6H-benzo[c]chromen-6-one

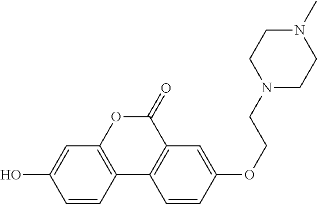 [SMILES: CN1CCN(CCOc2ccc3c(c2)c(=O)oc2cc(O)ccc23)CC1]

Acetyl chloride (0.046 ml, 0.64 mmol, 5.0 eq) was added to a solution of 3-((tert-butyldimethylsilyl)oxy)-8-(2-(4-methylpiperazin-1-yl)ethoxy)-6H-benzo[c]chromen-6-one (60 mg, 0.13 mmol, 1.0 eq) in MeOH (2 ml) at r.t. and the reaction mixture was stirred overnight. Methanol was evaporated under vacuum, the crude product was diluted with EtOAc and washed with a saturated solution of sodium carbonate. The aqueous layer was extracted with EtOAc, and the combined organic phases were dried over sodium sulfate. The crude product was purified by MPLC (SiO₂, MeOH/DCM 0% to 30%) to afford 3-hydroxy-8-(2-(4-methylpiperazin-1-yl)ethoxy)-6H-benzo[c]chromen-6-one (17 mg, 0.048 mmol, 37%). ¹H NMR (400 MHz, DMSO) δ 10.23 (br, 1H), 8.25-8.06 (m, 2H), 7.53 (d, J=2.7 Hz, 1H), 7.50 (dd, J=8.8, 2.9 Hz, 1H), 7.02-6.94 (m, 1H), 6.86-6.71 (m, 1H), 4.18 (dt, J=18.4, 5.7 Hz, 3H), 2.76-2.65 (m, 6H), 2.34-2.32 (m, 3H), 2.14 (s, 3H).

### Synthesis of (S)-3-hydroxy-8-((tetrahydrofuran-3-yl)oxy)-6H-benzo[c]chromen-6-one (31)

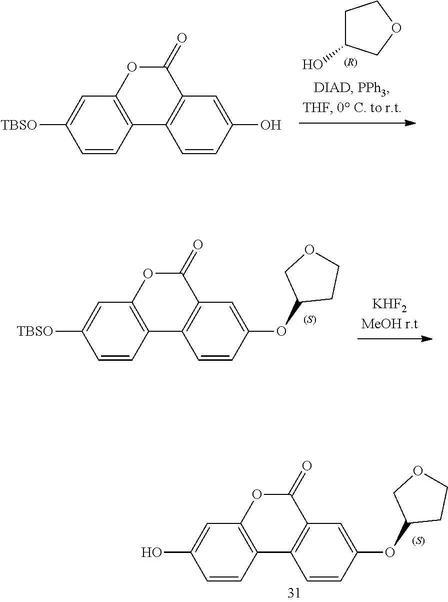

### Step 1: Synthesis of (S)-3-((tert-butyldimethylsilyl)oxy)-8-((tetrahydrofuran-3-yl)oxy)-6H-benzo[c]chromen-6-one

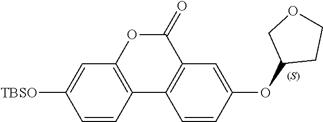

(S)-3-((tert-butyldimethylsilyl)oxy)-8-((tetrahydrofuran-3-yl)oxy)-6H-benzo[c]chromen-6-one was prepared (according to synthesis of 29) starting from C₂ (80 mg, 0.23 mmol) and (R)-tetrahydrofuran-3-ol (31 mg, 0.35 mmol) to afford (S)-3-((tert-butyldimethylsilyl)oxy)-8-((tetrahydrofuran-3-yl)oxy)-6H-benzo[c]chromen-6-one (45 mg, 47%) as yellowish oil. R_{f}=0.6 (EtOAc/hexane 1/1). ¹H NMR (400 MHz, CDCl₃) δ 8.00-7.80 (m, 2H), 7.74 (dd, J=23.0, 2.7 Hz, 1H), 7.33 (ddd, J=26.5, 8.8, 2.7 Hz, 1H), 6.90-6.80 (m, 2H), 5.09-4.95 (m, 1H), 4.13-3.88 (m, 4H), 2.43-2.07 (m, 2H), 1.02 (s, J=3.8 Hz, 9H), 0.27 (s, 3H), 0.25 (s, 3H).

### Step 2: Synthesis of (S)-3-hydroxy-8-((tetrahydrofuran-3-yl)oxy)-6H-benzo[c]chromen-6-one

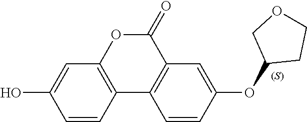

31 was prepared according to synthesis of 29 starting from (S)-3-((tert-butyldimethylsilyl)oxy)-8-((tetrahydrofuran-3-yl)oxy)-6H-benzo[c]chromen-6-one (40 mg, 0.097 mmol) and KHF₂ (27 mg, 0.34 mmol) to give (S)-3-hydroxy-8-((tetrahydrofuran-3-yl)oxy)-6H-benzo[c]chromen-6-one (22 mg, 76%) as a white solid. ¹H NMR (400 MHz, CDCl₃) δ 7.92-7.76 (m, 2H), 7.69 (s, 1H), 7.29 (ddd, J=11.3, 8.8, 2.8 Hz, 1H), 6.88-6.70 (m, 2H), 5.41-5.35 (m, 1H), 4.01-3.82 (m, 4H), 2.36-2.04 (m, 2H).

### Synthesis of 3-hydroxy-8-(2-(2-methoxyethoxy)ethoxy)-6H-benzo[c]chromen-6-one (32)

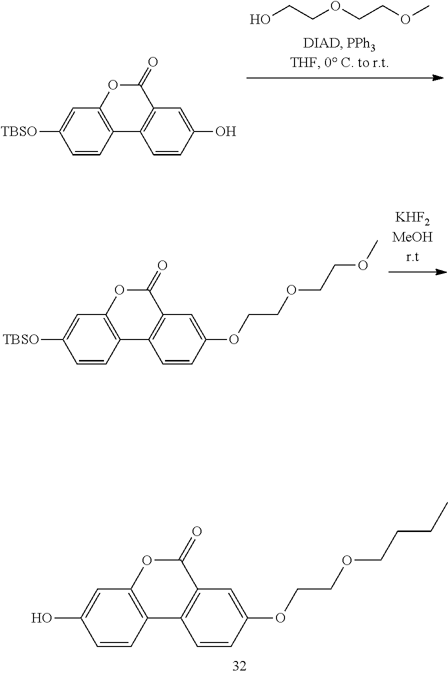

### Step 1: Synthesis of 3-((tert-butyldimethylsilyl)oxy)-8-(2-(2-methoxyethoxy)ethoxy)-6H-benzo[c]chromen-6-one

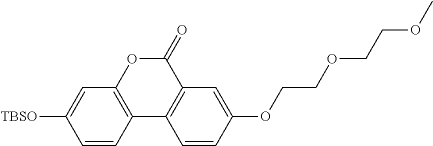 [SMILES: COCCOCCOc1ccc2c(c1)c(=O)oc1cc(O[Si](C)(C)C(C)(C)C)ccc12]

3-((tert-butyldimethylsilyl)oxy)-8-(2-(2-methoxyethoxy)ethoxy)-6H-benzo[c]chromen-6-one was prepared (according to synthesis of 29) starting from C2 (100 mg, 0.29 mmol) and 2-(2-methoxyethoxy)ethan-1-ol (42 mg, 0.35 mmol) to afford 3-((tert-butyldimethylsilyl)oxy)-8-(2-(2-methoxyethoxy)ethoxy)-6H-benzo[c]chromen-6-one (125 mg, 47%), which is contaminated with reduced DIAD as yellowish oil. R_{f}=0.5 (EtOAc/hexane 1/1). ¹H NMR (400 MHz, CDCl₃) δ 7.91-7.66 (m, 3H), 7.32-7.24 (m, 1H), 6.88-6.74 (m, 2H), 4.22-4.11 (m, 2H), 3.87-3.81 (m, 2H), 3.74-3.63 (m, 2H), 3.59-3.49 (m, 2H), 3.33 (s, 3H), 0.95-0.93 (m, 9H), 0.20 (s, 3H).

### Step 2: Synthesis of 3-hydroxy-8-(2-(2-methoxyethoxy)ethoxy)-6H-benzo[c]chromen-6-one

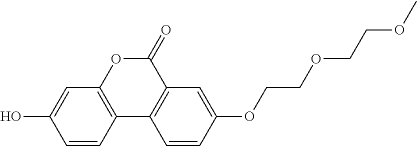 [SMILES: COCCOCCOc1ccc2c(c1)c(=O)oc1cc(O)ccc12]

32 was prepared according to synthesis of 29 starting from (100 mg, 0.220 mmol) and KHF₂ (70 mg, 0.90 mmol) to afford after purification by MPLC (SiO₂, EtAOc/hexane from 0% to 30%) 3-hydroxy-8-(2-(2-methoxyethoxy)ethoxy)-6H-benzo[c]chromen-6-one (24 mg, 32%) as a white solid as mixture of two compounds. ¹H NMR (400 MHz, CDCl₃) δ 7.75-7.56 (m, 2H), 7.34 (dd, J=42.4, 2.8 Hz, 1H), 7.21-6.48 (m, 3H), 4.05 (dt, J=14.8, 4.4 Hz, 2H), 3.92-3.85 (m, 2H), 3.80 (dt, J=6.1, 2.5 Hz, 2H), 3.70 (ddd, J=4.4, 3.5, 1.5 Hz, 2H), 3.46 (d, J=1.4 Hz, 3H).

### Synthesis of 3-hydroxy-8-((tetrahydro-2H-pyran-4-yl)oxy)-6H-benzo[c]chromen-6-one (33)

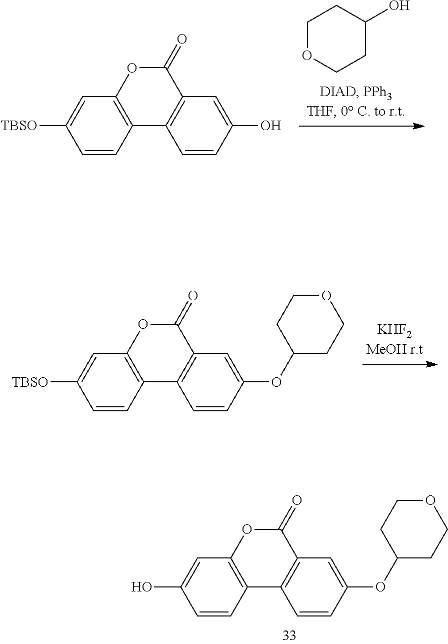 [SMILES: CC(C)(C)[Si](C)(C)Oc1ccc2c(c1)oc(=O)c1cc(O)ccc12 ; OC1CCOCC1 ; CC(C)(C)[Si](C)(C)Oc1ccc2c(c1)oc(=O)c1cc(OC3CCOCC3)ccc12 ; O=c1oc2cc(O)ccc2c2ccc(OC3CCOCC3)cc12]

### Step 1: Synthesis of 3-((tert-butyldimethylsilyl)oxy)-8-((tetrahydro-2H-pyran-4-yl)oxy)-6H-benzo[c]chromen-6-one

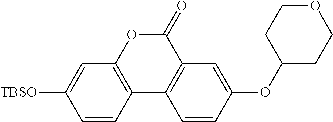 [SMILES: CC(C)(C)[Si](C)(C)Oc1ccc2c(c1)oc(=O)c1cc(OC3CCOCC3)ccc12]

Compound was prepared according to synthesis of 29 starting from C2 (100 mg, 0.29 mmol) and tetrahydro-2H-pyran-4-ol (36 mg, 0.35 mmol) to afford 3-((tert-butyldimethylsilyl)oxy)-8-((tetrahydro-2H-pyran-4-yl)oxy)-6H-benzo[c]chromen-6-one (64 mg, 51%) as yellowish oil. R_{f}=0.67 (EtOAc/hexane 4/6). ¹H NMR (400 MHz, CDCl₃) δ 7.98-7.81 (m, 2H), 7.77 (dd, J=7.3, 2.7 Hz, 1H), 7.42-7.27 (m, 1H), 6.96-6.80 (m, 2H), 4.61 (dtt, J=44.2, 7.8, 3.9 Hz, 1H), 4.01 (ddd, J=10.4, 5.9, 3.9 Hz, 2H), 3.62 (ddt, J=11.9, 7.8, 3.7 Hz, 2H), 2.06 (d, J=12.6 Hz, 2H), 1.83 (dtd, J=12.5, 8.2, 3.9 Hz, 2H), 1.01 (d, J=3.7 Hz, 9H), 0.32-0.20 (m, 6H).

### Step 2: Synthesis of 3-hydroxy-8-((tetrahydro-2H-pyran-4-yl)oxy)-6H-benzo[c]chromen-6-one

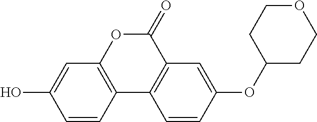 [SMILES: O=c1oc2cc(O)ccc2c2ccc(OC3CCOCC3)cc12]

33 was prepared starting from 3-((tert-butyldimethylsilyl)oxy)-8-((tetrahydro-2H-pyran-4-yl)oxy)-6H-benzo[c]chromen-6-one (60 mg, 0.14 mmol) and KHF₂ (38 mg, 0.49 mmol) to afford 3-hydroxy-8-((tetrahydro-2H-pyran-4-yl)oxy)-6H-benzo[c]chromen-6-one (29 mg, 66%) as a white solid. MS (ESI+): m/z=313. ¹H NMR (400 MHz, DMSO) δ 10.30-10.11 (m, 1H), 8.23-7.98 (m, 2H), 7.66-7.28 (m, 2H), 7.09-6.68 (m, 2H), 4.74 (dtt, J=25.7, 8.6, 4.0 Hz, 1H), 3.86 (dt, J=10.3, 4.2 Hz, 2H), 3.52 (tdd, J=11.6, 8.9, 2.7 Hz, 2H), 2.01 (dd, J=13.2, 3.5 Hz, 2H), 1.62 (dtt, J=14.1, 9.1, 4.6 Hz, 2H).

### Synthesis of 3-hydroxy-8-((tetrahydro-2H-pyran-3-yl)oxy)-6H-benzo[c]chromen-6-one (34)

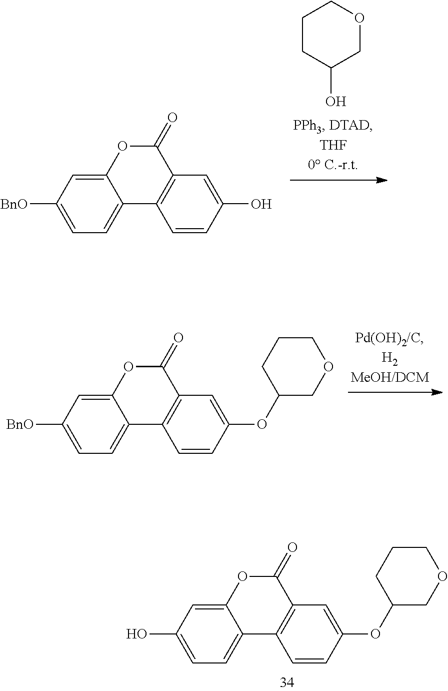 [SMILES: OC1CCCOC1 ; O=c1oc2cc(OCc3ccccc3)ccc2c2ccc(O)cc12 ; O=c1oc2cc(OCc3ccccc3)ccc2c2ccc(OC3CCCOC3)cc12 ; O=c1oc2cc(O)ccc2c2ccc(OC3CCCOC3)cc12]

### Step 1: Synthesis of 3-(benzyloxy)-8-((tetrahydro-2H-pyran-3-yl)oxy)-6H-benzo[c]chromen-6-one

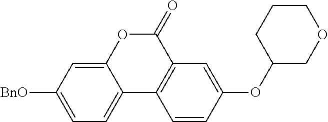 [SMILES: O=c1oc2cc(OCc3ccccc3)ccc2c2ccc(OC3CCCOC3)cc12]

3-(benzyloxy)-8-hydroxy-6H-benzo[c]chromen-6-one (64 mg, 0.20 mmol, 1.0 eq.) was dissolved in THF (0.7 mL) in a 10 mL Biotage MW vial. Subsequently PPh₃ (79 mg, 0.30 mmol, 1.5 eq.) and Tetrahydro-2H-pyran-3-ol (31 mg, 0.30 mmol, 1.5 eq.) were added and the reaction mixture was cooled to 0° C. in an ice-bath in which it was stirred for 5 min. Then a solution of Di-tert-butyl-diazene-1,2-dicarboxylate (69 mg, 0.30 mmol, 1.5 eq.) (DTAD) in THF (0.1 mL) was added dropwise to the reaction mixture. Upon complete addition the reaction mixture turned dark yellow and stirring was continued overnight at r.t. After overnight stirring starting material was still present, therefore PPh₃ (79 mg, 0.30 mmol, 1.5 eq.), Tetrahydro-2H-pyran-3-ol (31 mg, 0.30 mmol, 1.5 eq.) and a solution of Di-tert-butyl-diazene-1,2-dicarboxylate (DTAD) in THF (0.1 mL) were added to reaction mixture to bring the reaction to completion. After an additional 2 h of stirring at room temperature the reaction mixture was concentrated under vacuum and loaded on silica to be purified by MPLC (SiO₂, 12 g, EtOAc in Hex 0-35%) to afford 3-(benzyloxy)-8-((tetrahydro-2H-pyran-3-yl)oxy)-6H-benzo[c]chromen-6-one (50 mg, 62%) as a light yellow solid. ¹H NMR (400 MHz, CDCl₃) δ 7.92 (d, J=8.9 Hz, 1H), 7.86 (d, J=8.9 Hz, 1H), 7.77 (d, J=2.8 Hz, 1H), 7.46-7.31 (m, 5H), 6.97 (dd, J=8.8, 2.6 Hz, 1H), 6.92 (d, J=2.5 Hz, 1H), 5.12 (s, 2H), 4.47 (tt, J=6.8, 3.5 Hz, 1H), 3.95 (ddd, J=11.6, 3.2, 1.2 Hz, 1H), 3.75 (ddd, J=10.6, 6.2, 3.9 Hz, 1H), 3.70-3.60 (m, 2H), 2.12 (tt, J=11.8, 6.0 Hz, 1H), 1.89 (dddt, J=31.0, 17.3, 8.0, 3.9 Hz, 3H), 1.70-1.59 (m, 1H).

### Step 2: Synthesis of 3-hydroxy-8-((tetrahydro-2H-pyran-3-yl)oxy)-6H-benzo[c]chromen-6-one

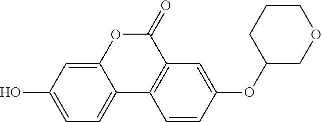 [SMILES: O=c1oc2cc(O)ccc2c2ccc(OC3CCCOC3)cc12]

3-(benzyloxy)-8-((tetrahydro-2H-pyran-3-yl)oxy)-6H-benzo[c]chromen-6-one (50 mg, 0.12 mmol, 1.0 eq.) was dissolved in MeOH/DCM (5 mL, 10/1) and Pd(OH)₂/C (20 mg) was added in one portion. Then the reaction mixture was evacuated and backfilled with N₂ three times before putting it under hydrogen atmosphere (balloon). The reaction mixture was stirred for 2 h and upon complete consumption of starting material (as indicated by TLC) filtered over silica and concentrated under vacuum to afford the crude product which was loaded on silica and purified by MPLC (SiO₂, 12 g, EtOAc in Hex 0-50%) to obtain 3-hydroxy-8-((tetrahydro-2H-pyran-3-yl)oxy)-6H-benzo[c]chromen-6-one (33 mg, 0.11 mmol, 89%) as a white solid. ¹H NMR (400 MHz, DMSO) δ 10.21 (s, 1H), 8.20 (d, J=9.0 Hz, 1H), 8.09 (d, J=8.8 Hz, 1H), 7.61 (d, J=2.8 Hz, 1H), 7.53 (dd, J=8.9, 2.8 Hz, 1H), 6.82 (dd, J=8.7, 2.4 Hz, 1H), 6.74 (d, J=2.4 Hz, 1H), 4.57 (dt, J=6.2, 3.2 Hz, 1H), 3.84 (dd, J=11.6, 2.1 Hz, 1H), 3.64 (ddd, J=10.8, 6.5, 3.7 Hz, 1H), 3.56 (dd, J=11.7, 5.6 Hz, 2H), 2.05 (dd, J=8.9, 4.9 Hz, 1H), 1.87-1.68 (m, 2H), 1.63-1.48 (m, 1H).

### Synthesis of 3-hydroxy-8-((tetrahydrofuran-3-yl)oxy)-6H-benzo[c]chromen-6-one (35)

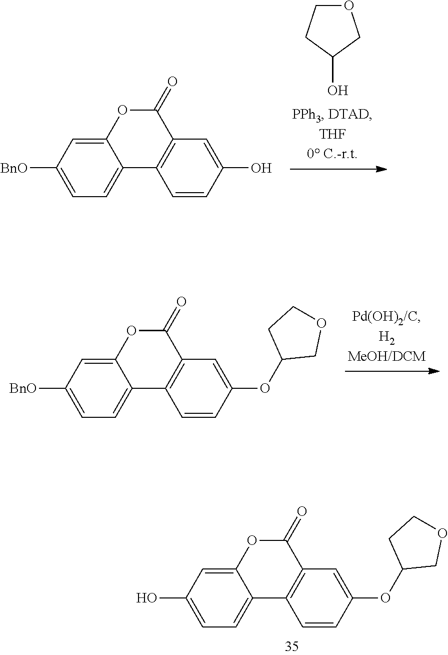 [SMILES: O=c1oc2cc(OCc3ccccc3)ccc2c2ccc(O)cc12 ; OC1CCOC1 ; O=c1oc2cc(OCc3ccccc3)ccc2c2ccc(OC3CCOC3)cc12 ; O=c1oc2cc(O)ccc2c2ccc(OC3CCOC3)cc12]

### Step 1: Synthesis of 3-(benzyloxy)-8-((tetrahydrofuran-3-yl)oxy)-6H-benzo[c]chromen-6-one

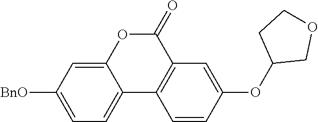 [SMILES: O=c1oc2cc(OCc3ccccc3)ccc2c2ccc(OC3CCOC3)cc12]

3-(benzyloxy)-8-hydroxy-6H-benzo[c]chromen-6-one (100 mg, 0.31 mmol, 1.0 eq.) was dissolved in THF (1.1 mL). Subsequently PPh₃ (124 mg, 0.470 mmol, 1.5 eq.) and Tetrahydrofuran-3-ol (42 mg, 0.47 mmol, 1.5 eq.) were added and the reaction mixture was cooled to 0° C. in an ice-bath in which it was stirred for 5 min. Then a solution of Di-tert-butyl-diazene-1,2-dicarboxylate (109 mg, 0.470 mmol, 1.5 eq.) (DTAD) in THF (0.2 mL) was added dropwise to the reaction mixture. Upon complete addition the reaction mixture turned dark yellow and stirring was continued overnight at r.t. After overnight stirring starting material was still present, therefore PPh₃ (124 mg, 0.470 mmol, 1.5 eq.), Tetrahydrofuran-3-ol (42 mg, 0.47 mmol, 1.5 eq.) and a solution of Di-tert-butyl-diazene-1,2-dicarboxylate (109 mg, 0.470 mmol, 1.5 eq.) (DTAD) in THF (0.2 mL) were added to reaction mixture to bring the reaction to completion. After an additional 2 h of stirring at room temperature the reaction mixture was concentrated under vacuum and loaded on silica to be purified by MPLC (SiO₂, 12 g, EtOAc in Hex 0-35%) to afford 3-(benzyloxy)-8-((tetrahydrofuran-3-yl)oxy)-6H-benzo[c]chromen-6-one (100 mg, 82%) as a light yellow solid. The NMR after purification still showed a significant amount of reduced DTAD but the reaction was taken to the next step crude, therefore the NMR is not reported here.

### Step 2: Synthesis of 3-hydroxy-8-((tetrahydrofuran-3-yl)oxy)-6H-benzo[c]chromen-6-one

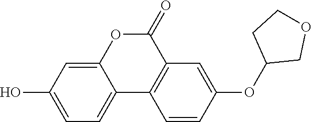 [SMILES: O=c1oc2cc(O)ccc2c2ccc(OC3CCOC3)cc12]

3-(benzyloxy)-8-((tetrahydrofuran-3-yl)oxy)-6H-benzo[c]chromen-6-one (100 mg, 0.260 mmol, 1.0 eq.) was dissolved in MeOH/DCM (7 mL, 10/1) and Pd(OH)₂/C (60 mg) was added in one portion. Then the reaction mixture was evacuated and backfilled with N₂ three times before putting it under hydrogen atmosphere with the use of a balloon. The reaction mixture was stirred for 2 h and upon complete consumption of starting material (as indicated by TLC) filtered over silica and concentrated under reduced pressure to give the crude product which was loaded on silica and purified by flash column chromatography (SiO₂, 12 g, EtOAc in Hex 0-50%) to give 3-hydroxy-8-((tetrahydrofuran-3-yl)oxy)-6H-benzo[c]chromen-6-one (65 mg, 72%) as a white solid. ¹H NMR (400 MHz, DMSO) δ 10.21 (s, 1H), 8.22 (d, J=8.8 Hz, 1H), 8.09 (d, J=8.8 Hz, 1H), 7.57 (d, J=2.7 Hz, 1H), 7.49 (dd, J=8.9, 2.8 Hz, 1H), 6.83 (dd, J=8.7, 2.4 Hz, 1H), 6.75 (d, J=2.4 Hz, 1H), 5.25-5.19 (m, 1H), 3.92 (dd, J=10.2, 4.4 Hz, 1H), 3.88-3.83 (m, 2H), 3.78 (td, J=8.4, 4.6 Hz, 1H), 2.36-2.20 (m, 1H), 2.02 (dd, J=14.2, 7.5 Hz, 1H).

### Synthesis of 3-hydroxy-8-(oxetan-3-yloxy)-6H-benzo[c]chromen-6-one (36)

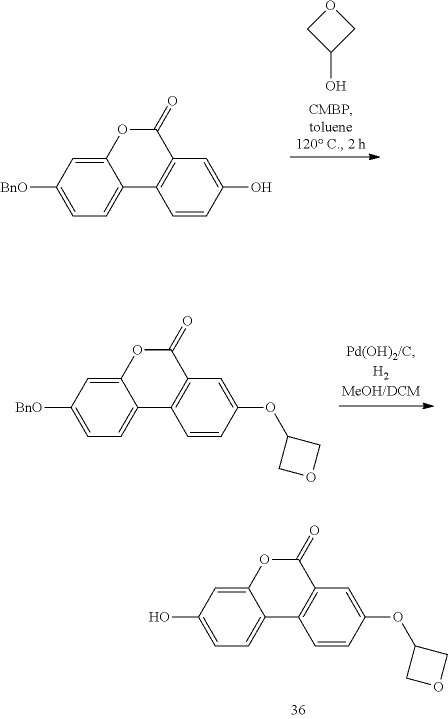 [SMILES: OC1COC1 ; O=c1oc2cc(OCc3ccccc3)ccc2c2ccc(O)cc12 ; O=c1oc2cc(OCc3ccccc3)ccc2c2ccc(OC3COC3)cc12 ; O=c1oc2cc(O)ccc2c2ccc(OC3COC3)cc12]

### Step 1: Synthesis of 3-(benzyloxy)-8-(oxetan-3-yloxy)-6H-benzo[c]chromen-6-one

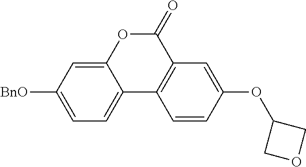 [SMILES: O=c1oc2cc(OCc3ccccc3)ccc2c2ccc(OC3COC3)cc12]

Cyanomehtylenetributylphosphorane (150 mg, 0.630 mmol, 2.5 eq.) was added at r.t. to a solution of 3-(benzyloxy)-8-hydroxy-6H-benzo[c]chromen-6-one (80 mg, 0.25 mmol, 1.0 eq.) and oxetan-3ol (56 mg, 0.75 mmol, 3.0 eq.) in toluene (1.3 mL) in one portion and the reaction mixture was heated to 120° C. for 2 h in a sealed vial. After the full conversion of starting material, the reaction mixture was allowed to cool down to r.t., concentrated and loaded on silica to be purified by MPLC (SiO₂, 12 g, EtOAc in Hex 0-30%) to afford 3-(benzyloxy)-8-(oxetan-3-yloxy)-6H-benzo[c]chromen-6-one (74 mg, 79%) as a light yellow foam. ¹H NMR (400 MHz, CDCl³) δ 7.96 (d, J=8.8 Hz, 1H), 7.88 (d, J=8.9 Hz, 1H), 7.48-7.34 (m, 7H), 6.99 (dd, J=8.8, 2.6 Hz, 1H), 6.94 (d, J=2.5 Hz, 1H), 5.39-5.29 (m, 1H), 5.14 (s, 2H), 5.07 (ddd, J=7.1, 6.0, 0.9 Hz, 2H), 4.79 (ddd, J=7.4, 5.0, 1.0 Hz, 2H).

### Step 2: Synthesis of 3-hydroxy-8-(oxetan-3-yloxy)-6H-benzo[c]chromen-6-one

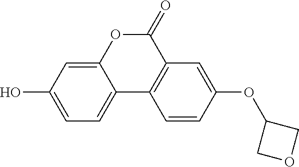 [SMILES: O=c1oc2cc(O)ccc2c2ccc(OC3COC3)cc12]

3-(benzyloxy)-8-(oxetan-3-yloxy)-6H-benzo[c]chromen-6-one (70 mg, 0.19 mmol, 1.0 eq.) was dissolved in MeOH/DCM (5 mL, 10/1) and Pd(OH)₂/C (15 mg) was added in one portion. Then the reaction mixture was evacuated and backfilled with N₂ three times before putting it under hydrogen atmosphere with the use of a balloon. The reaction mixture was stirred for 4 h and upon complete consumption of starting material (as indicated by TLC) filtered over silica and concentrated under reduced pressure to give the crude product which was loaded on silica and purified by flash column chromatography (SiO₂, 12 g, EtOAc in Hex 0-50%) to afford 3-hydroxy-8-(oxetan-3-yloxy)-6H-benzo[c]chromen-6-one (25 mg, 0.09 mmol, 47%) as a white solid. ¹H NMR (400 MHz, DMSO) δ 10.24 (s, 1H), 8.24 (d, J=8.9 Hz, 1H), 8.10 (d, J=8.8 Hz, 1H), 7.45 (dd, J=8.8, 2.8 Hz, 1H), 7.35 (d, J=2.8 Hz, 1H), 6.83 (dd, J=8.7, 2.4 Hz, 1H), 6.75 (d, J=2.4 Hz, 1H), 5.47 (q, J=5.4, 4.8 Hz, 1H), 4.98 (t, J=7.0 Hz, 2H), 4.59 (dd, J=7.7, 5.1 Hz, 2H).

### Synthesis of 8-((2-oxaspiro[3.3]heptan-6-yl)oxy)-3-hydroxy-6H-benzo[c]chromen-6-one (37)

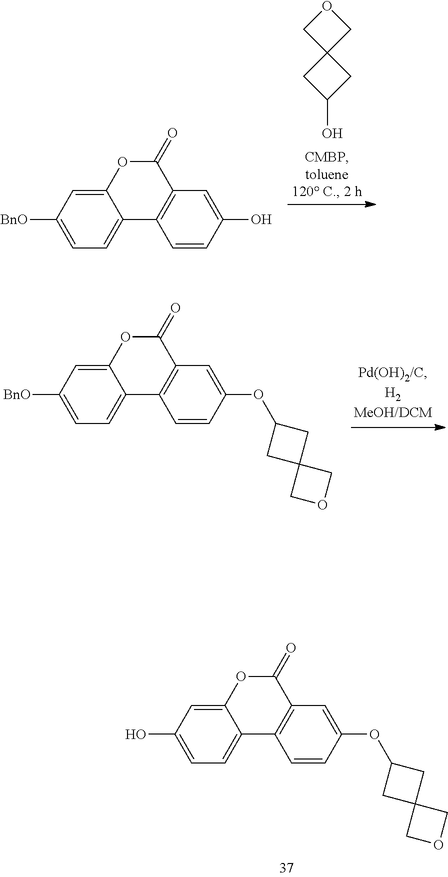 [SMILES: O=c1oc2cc(OCc3ccccc3)ccc2c2ccc(O)cc12 ; OC1CC2(COC2)C1 ; O=c1oc2cc(OCc3ccccc3)ccc2c2ccc(OC3CC4(COC4)C3)cc12 ; O=c1oc2cc(O)ccc2c2ccc(OC3CC4(COC4)C3)cc12]

### Step 1: Synthesis of 8-((2-oxaspiro[3.3]heptan-6-yl)oxy)-3-(benzyloxy)-6H-benzo[c]chromen-6-one

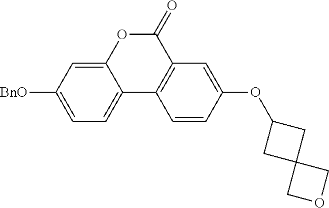 [SMILES: O=c1oc2cc(OCc3ccccc3)ccc2c2ccc(OC3CC4(COC4)C3)cc12]

Cyanomehtylenetributylphosphorane (95 mg, 0.39 mmol, 2.5 eq.) was added at r.t. to a solution of 3-(benzyloxy)-8-hydroxy-6H-benzo[c]chromen-6-one (50 mg, 0.16 mmol, 1.0 eq.) and 2-oxaspiro[3.3]heptan-6-ol (39 mg, 0.35 mmol, 2.2 eq.) in toluene (3.0 mL) in one portion and the reaction mixture was heated to 120° C. for 2 h in a sealed vial. After the full conversion of starting material, the reaction mixture was allowed to cool down to r.t., concentrated and loaded on silica to be purified by flash column chromatography (SiO₂, 12 g, EtOAc in Hex 0-30%) to afford 8-((2-oxaspiro[3.3]heptan-6-yl)oxy)-3-(benzyloxy)-6H-benzo[c]chromen-6-one (40 mg, 0.10 mmol, 61%) as a light yellow solid. ¹H NMR (400 MHz, DMSO) δ 8.26 (d, J=8.9 Hz, 1H), 8.20 (d, J=8.9 Hz, 1H), 7.54-7.32 (m, 7H), 7.09 (d, J=2.5 Hz, 1H), 7.06 (dd, J=8.7, 2.6 Hz, 1H), 5.22 (s, 2H), 4.77 (p, J=6.8 Hz, 1H), 4.66 (s, 2H), 4.55 (s, 2H), 2.88-2.78 (m, 2H), 2.33-2.24 (m, 2H).

### Step 2: Synthesis of 8-((2-oxaspiro[3.3]heptan-6-yl)oxy)-3-hydroxy-6H-benzo[c]chromen-6-one

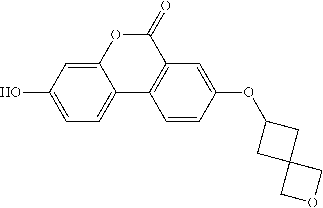 [SMILES: O=c1oc2cc(O)ccc2c2ccc(OC3CC4(COC4)C3)cc12]

8-((2-oxaspiro[3.3]heptan-6-yl)oxy)-3-(benzyloxy)-6H-benzo[c]chromen-6-one (40 mg, 0.10 mmol, 1.0 eq.) was dissolved in MeOH/DCM (5 mL, 10/1) and Pd(OH)₂/C (14 mg) was added in one portion. Then the reaction mixture was evacuated and backfilled with N₂ three times before putting it under hydrogen atmosphere with the use of a balloon. The reaction mixture was stirred for 4 h and upon complete consumption of starting material (as indicated by TLC) filtered over silica and concentrated under vacuum to afford the crude product which was loaded on silica and purified by flash column chromatography (SiO₂, 12 g, EtOAc in Hex 0-50%) to give 8-((2-oxaspiro[3.3]heptan-6-yl)oxy)-3-hydroxy-6H-benzo[c]chromen-6-one (26 mg, 0.08 mmol, 83%) as a white solid. MS (ESI+): m/z=325. ¹H NMR (400 MHz, DMSO) δ 10.23 (s, 1H), 8.19 (d, J=8.9 Hz, 1H), 8.08 (d, J=8.8 Hz, 1H), 7.46 (d, J=2.8 Hz, 1H), 7.41 (dd, J=8.8, 2.8 Hz, 1H), 6.82 (dd, J=8.7, 2.4 Hz, 1H), 6.74 (d, J=2.4 Hz, 1H), 4.76 (q, J=6.8 Hz, 1H), 4.66 (s, 2H), 4.55 (s, 2H), 2.87-2.76 (m, 2H), 2.32-2.18 (m, 2H).

In a similar fashion, the 9-substituted analogue 38 was prepared according to the scheme below:

### Synthesis of 3-hydroxy-9-((tetrahydro-2H-pyran-4-yl)oxy)-6H-benzo[c]chromen-6-one (38)

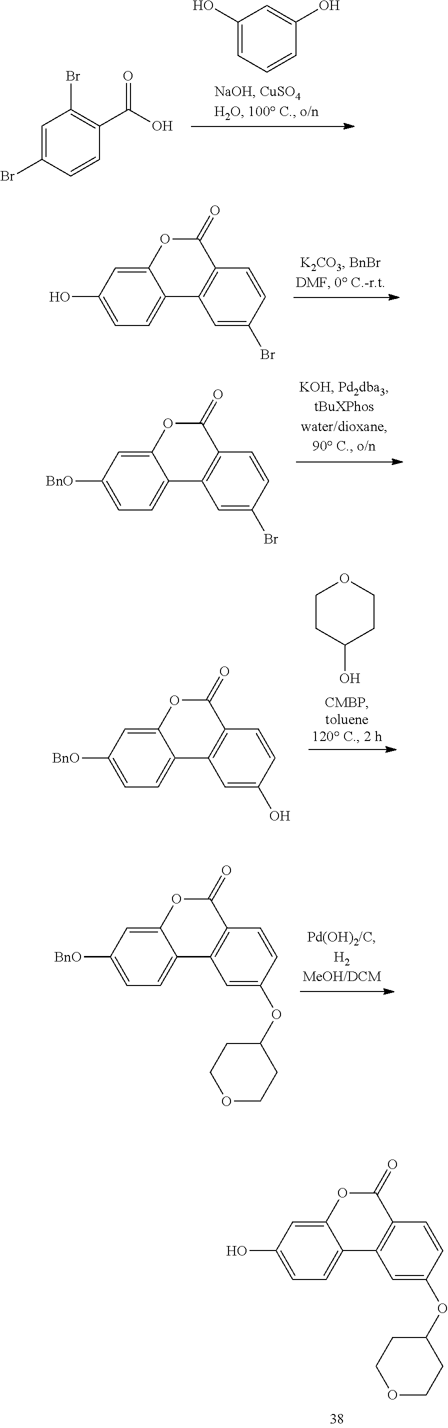

### Step 1: Synthesis of 9-bromo-3-hydroxy-6H-benzo[c]chromen-6-one

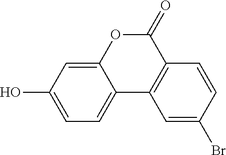 [SMILES: O=c1oc2cc(O)ccc2c2cc(Br)ccc12]

A mixture of 2,4-dibromobenzoic acid (5.00 g, 17.9 mmol, 1.0 eq.), resorcinol (3.93 g, 35.7 mmol, 2.0 eq.) and sodium hydroxide (1.71 g, 42.9 mmol, 2.4 eq.) in water (15 ml) was heated under reflux for 60 minutes. After the addition of copper sulfate (5% aqueous solution, 10 mL), the mixture was refluxed again overnight and a precipitate was formed which was filtered off and washed with HCl (1M) then dried under vacuum to give 9-bromo-3-hydroxy-6H-benzo[c]chromen-6-one (2.91 g, 56%) as an ochrey solid. ¹H NMR (400 MHz, DMSO) δ 10.44 (s, 1H), 8.47 (s, 1H), 8.19 (d, J=8.7 Hz, 1H), 8.04 (d, J=8.4 Hz, 1H), 7.69 (d, J=8.4 Hz, 1H), 6.86-6.78 (m, 1H), 6.73 (s, 1H).

### Step 2: Synthesis of 3-(benzyloxy)-9-bromo-6H-benzo[c]chromen-6-one

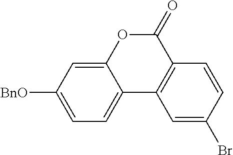 [SMILES: O=c1oc2cc(OCc3ccccc3)ccc2c2cc(Br)ccc12]

To a suspension of 9-bromo-3-hydroxy-6H-benzo[c]chromen-6-one (2.00 mg, 6.87 mmol, 1.0 eq.) in DMF (35 mL) was added in one portion K₂CO₃ (2.09 g, 15.1 mmol, 2.2 eq.). The suspension was cooled to 0° C. and stirred for 5 min. Benzyl bromide (1.41 g, 8.24 mmol, 1.2 eq.) was added dropwise over a period of 5 min and upon complete addition the reaction mixture was stirred at 0° C. for 10 min before being allowed to warm up to room temperature over 2 h. After the complete consumption of starting material (as indicated by TLC) the reaction mixture was quenched with half-saturated aqueous sodium bicarbonate solution. The precipitate was filtered over a Buchner funnel, washed with hexanes and dried to afford 3-(benzyloxy)-9-bromo-6H-benzo[c]chromen-6-one (1.49 g, 61%) as a light brown solid. ¹H NMR (400 MHz, CDCl₃) δ 8.20 (d, J=8.7 Hz, 1H), 7.78 (d, J=8.9 Hz, 1H), 7.41-7.28 (m, 6H), 6.91-6.88 (m, 2H), 6.85 (d, J=2.5 Hz, 1H), 5.07 (s, 2H).

### Step 3: Synthesis of 3-(benzyloxy)-9-hydroxy-6H-benzo[c]chromen-6-one

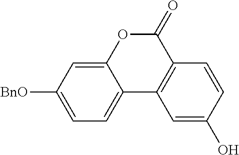 [SMILES: O=c1oc2cc(OCc3ccccc3)ccc2c2cc(O)ccc12]

3-(benzyloxy)-9-bromo-6H-benzo[c]chromen-6-one (800 mg, 2.10 mmol, 1.0 eq) was suspended in 1,4-dioxane (7 mL) in a 20 mL Biotage MW vial. To this suspension was added Pd₂dba₃ (49 mg, 0.21 mmol, 0.1 eq.) followed by tBuXPhos (200 mg, 0.42 mmol, 0.2 eq.). Subsequently the MW vial was sealed and degassed with nitrogen for 10 min. Then, a solution of KOH (471 mg, 8.39 mmol, 4.4 eq.) in H₂O (3 mL) was added slowly to the reaction mixture, and put in a pre-heated oil bath at 90° C. for 3 h. Upon complete consumption of starting material (as indicated by TLC) the reaction mixture was cooled to 0° C. and the pH adjusted to 1 with 6M aqueous HCl. The mixture was extracted with ethyl acetate (3×10 mL) and the combined organic phases were dried over anhydrous Na₂SO₄ and concentrated under vacuum. The crude material was purified by MPLC (SiO₂, 40 g, EtOAc in Hexanes 0-30%) to afford 3-(benzyloxy)-9-hydroxy-6H-benzo[c]chromen-6-one (225 mg, 37%) as a light yellow solid. ¹H NMR (400 MHz, CDCl₃) δ 8.20 (d, J=8.7 Hz, 1H), 7.78 (d, J=8.9 Hz, 1H), 7.41-7.28 (m, 6H), 6.91-6.88 (m, 2H), 6.85 (d, J=2.5 Hz, 1H), 5.07 (s, 2H).

### Step 4: Synthesis of 3-(benzyloxy)-9-((tetrahydro-2H-pyran-4-yl)oxy)-6H-benzo[c]chromen-6-one

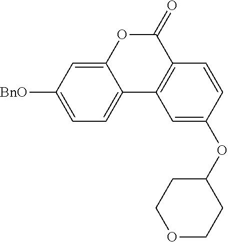 [SMILES: O=c1oc2cc(OCc3ccccc3)ccc2c2cc(OC3CCOCC3)ccc12]

Cyanomehtylenetributylphosphorane (227 mg, 0.940 mmol, 2.5 eq.) was added at r.t. to a solution of 3-(benzyloxy)-9-hydroxy-6H-benzo[c]chromen-6-one (120 mg, 0.380 mmol, 1.0 eq.) and tetrahydro-2H-pyran-4-ol (77 mg, 0.71 mmol, 2.0 eq.) in toluene (3.8 mL) in one portion and the reaction mixture was heated to 120° C. for 2 h in a sealed vial. After the full conversion of starting material, the reaction mixture was allowed to cool down to r.t., concentrated and loaded on silica to be purified by MPLC (SiO₂, 12 g, EtOAc in Hex 0-30%) to afford 3-(benzyloxy)-9-((tetrahydro-2H-pyran-4-yl)oxy)-6H-benzo[c]chromen-6-one (135 mg, 89%) as a light yellow foam. ¹H NMR (400 MHz, CDCl₃) δ 8.28 (d, J=8.8 Hz, 1H), 7.86 (d, J=8.9 Hz, 1H), 7.51-7.32 (m, 6H), 7.03 (dd, J=8.9, 2.4 Hz, 1H), 6.97 (dd, J=8.8, 2.6 Hz, 1H), 6.91 (d, J=2.5 Hz, 1H), 5.13 (s, 2H), 4.73 (tt, J=7.7, 3.8 Hz, 1H), 4.02 (ddd, J=11.8, 6.3, 3.8 Hz, 2H), 3.65 (ddd, J=11.5, 8.1, 3.3 Hz, 2H), 2.17-2.05 (m, 2H), 1.94-1.82 (m, 2H).

### Step 5: Synthesis of 3-hydroxy-9-((tetrahydro-2H-pyran-4-yl)oxy)-6H-benzo[c]chromen-6-one

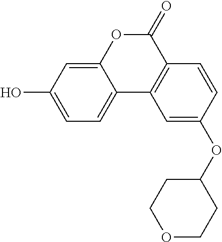 [SMILES: O=c1oc2cc(O)ccc2c2cc(OC3CCOCC3)ccc12]

3-(benzyloxy)-9-((tetrahydro-2H-pyran-4-yl)oxy)-6H-benzo[c]chromen-6-one (135 mg, 0.340 mmol, 1.0 eq.) was dissolved in MeOH/DCM (10 mL, 10/1) and Pd(OH)₂/C (70 mg) was added in one portion. Then the reaction mixture was evacuated and backfilled with N₂ three times before putting it under hydrogen atmosphere (balloon). The reaction mixture was stirred for 4 h and upon complete consumption of starting material (as indicated by TLC) filtered over silica and concentrated under vacuum to afford the crude product which was loaded on silica and purified by MPLC (SiO₂, 12 g, EtOAc in Hex 0-50%) to afford 3-hydroxy-9-((tetrahydro-2H-pyran-4-yl)oxy)-6H-benzo[c]chromen-6-one (40 mg, 34%) as a white solid. ¹H NMR (400 MHz, DMSO) δ 10.33 (s, 1H), 8.25 (d, J=8.9 Hz, 1H), 8.11 (d, J=8.9 Hz, 1H), 7.72 (d, J=2.4 Hz, 1H), 7.17 (dd, J=8.9, 2.4 Hz, 1H), 6.83 (dd, J=8.7, 2.4 Hz, 1H), 6.73 (d, J=2.4 Hz, 1H), 4.97 (tt, J=8.6, 4.1 Hz, 1H), 3.90 (dt, J=11.7, 4.3 Hz, 2H), 3.56 (ddd, J=11.8, 9.6, 2.7 Hz, 2H), 2.07 (dd, J=11.3, 7.7 Hz, 2H), 1.66 (ddt, J=13.7, 9.1, 4.6 Hz, 2H).

### G) Ester “A” ring analogues with alkynyl substitution prepared by Sonogashira reaction

### Synthesis of 3-hydroxy-8-(3-hydroxyprop-1-yn-1-yl)-6H-benzo[c]chromen-6-one (39)

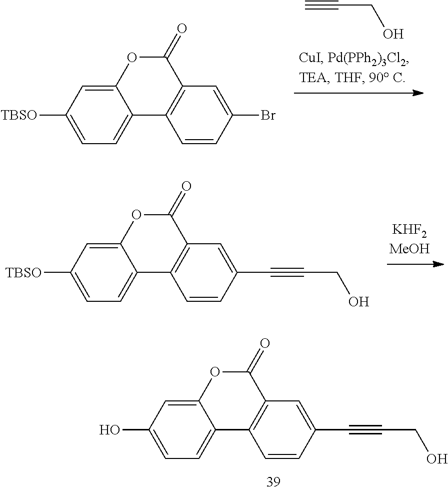

### Step 1: Synthesis of 3-((tert-butyldimethylsilyl)oxy)-8-(3-hydroxyprop-1-yn-1-yl)-6H-benzo[c]chromen-6-one

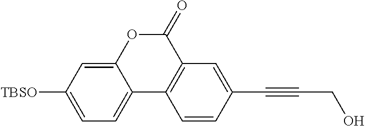 [SMILES: CC(C)(C)[Si](C)(C)Oc1ccc2c(c1)oc(=O)c1cc(C#CCO)ccc12]

To a solution of S8-bromo-3-((tert-butyldimethylsilyl)oxy)-6H-benzo[c]chromen-6-one (1.45 g, 3.58 mmol, 1.0 eq.) in THF (50 mL) in a 250 mL flask was subsequently added propargyl alcohol (501 mg, 8.94 mmol, 2.5 eq.), Pd(PPh₃)₂Cl₂ (251 mg, 0.360 mmol, 0.1 eq.) and CuI (68 mg, 0.36 mmol, 0.1 eq.) and the reaction was degassed at r.t. with N₂ for 10 min. Triethylamine (724 mg, 7.15 mmol, 2.0 eq.) was added in one portion and the reaction mixture was put into a pre-heated oil-bath at 90° C. Upon full conversion of the starting material (as indicated by TLC) the reaction mixture was allowed to cool to r.t. and quenched with water and extracted with EtOAc (2×100 mL). The combined organic layers were dried over anhydrous Na₂SO₄ and concentrated under vacuum. The crude product was purified by MPLC (SiO₂, 80 g, EtOAc in Hex 0-40%) to afford 3-((tert-butyldimethylsilyl)oxy)-8-(3-hydroxyprop-1-yn-1-yl)-6H-benzo[c]chromen-6-one (490 mg, 36%) as a brownish solid. ¹H NMR (400 MHz, CDCl₃) δ 8.41 (d, J=1.8 Hz, 1H), 7.94 (d, J=8.4 Hz, 1H), 7.88 (d, J=8.5 Hz, 1H), 7.79 (dd, J=8.3, 1.8 Hz, 1H), 6.94-6.80 (m, 2H), 4.54 (d, J=6.1 Hz, 2H), 1.00 (s, 9H), 0.26 (s, 6H).

### Step 2: Synthesis of 3-hydroxy-8-(3-hydroxyprop-1-yn-1-yl)-6H-benzo[c]chromen-6-one

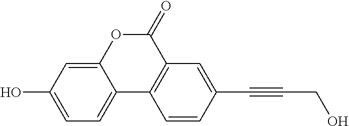 [SMILES: O=c1oc2cc(O)ccc2c2ccc(C#CCO)cc12]

3-((tert-butyldimethylsilyl)oxy)-8-(3-hydroxyprop-1-yn-1-yl)-6H-benzo[c]chromen-6-one (160 mg, 0.420 mmol, 1.0 eq.) was dissolved in MeOH (2 mL) and cooled to r.t. in an ice-bath and the resulting yellow solution was stirred for 10 min. Then KHF₂ (66 mg, 0.82 mmol, 2.0 eq.) was added in on portion and the reaction was stirred at room temperature overnight. Upon complete consumption of the starting material (as indicated by TLC), the reaction mixture was filtered over a glass frit (Por.4) and the filter residue was washed with MeOH and dried under vacuum to afford 3-hydroxy-8-(3-methoxyprop-1-yn-1-yl)-6H-benzo[c]chromen-6-one (112 mg, 0.420 mmol, 99%) as a pale brown solid. ¹H NMR (400 MHz, DMSO) δ 10.45 (s, 1H), 8.26 (d, J=8.4 Hz, 1H), 8.15 (d, J=8.8 Hz, 1H), 8.12 (d, J=1.8 Hz, 1H), 7.87 (dd, J=8.4, 1.9 Hz, 1H), 6.85 (dd, J=8.7, 2.4 Hz, 1H), 6.75 (d, J=2.4 Hz, 1H), 5.41 (s, 1H), 4.35 (s, 2H).

Additionally, the hydrogenation of the above compound was carried out as described below:

### Step 3: Synthesis of 3-hydroxy-8-(3-hydroxypropyl)-6H-benzo[c]chromen-6-one (40)

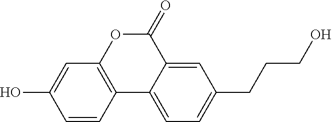 [SMILES: O=c1oc2cc(O)ccc2c2ccc(CCCO)cc12]

3-hydroxy-8-(3-methoxyprop-1-yn-1-yl)-6H-benzo[c]chromen-6-one (86 mg, 0.32 mmol, 1.0 eq.) and Pd(OH)₂/C (9 mg, 0.07 mmol. 0.2 eq.) in MeOH (5 ml) was hydrogenated under atmospheric pressure overnight. The reaction mixture was filtered over a pad of celite and the solvent was evaporated concentrate under vacuum to afford 3-hydroxy-8-(3-hydroxypropyl)-6H-benzo[c]chromen-6-one (70 mg, 80%) as a white solid. MS (ESI+): m/z=271. ¹H NMR (400 MHz, DMSO) δ 8.13 (d, J=8.3 Hz, 1H), 8.04 (dd, J=8.8, 2.2 Hz, 1H), 7.97 (d, J=1.9 Hz, 1H), 7.70 (dd, J=8.3, 2.0 Hz, 1H), 6.76 (dd, J=8.7, 2.4 Hz, 1H), 6.64 (d, J=2.5 Hz, 1H), 4.52 (s, 1H), 3.43 (t, J=6.4 Hz, 2H), 2.81-2.71 (m, 2H), 1.86-1.73 (m, 2H).

### Synthesis of 3-hydroxy-8-(3-methoxyprop-1-yn-1-yl)-6H-benzo[c]chromen-6-one (41)

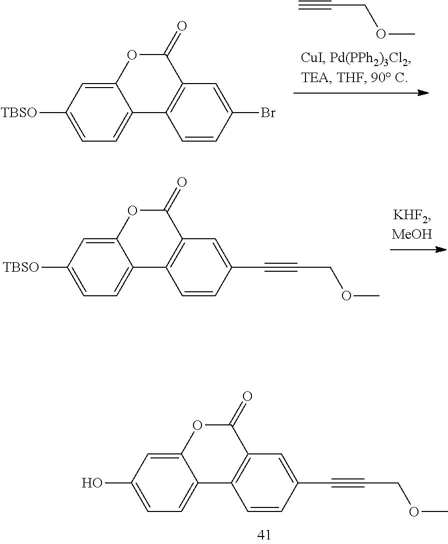

### Step 1: Synthesis of 3-((tert-butyldimethylsilyl)oxy)-8-(3-methoxyprop-1-yn-1-yl)-6H-benzo[c]chromen-6-one

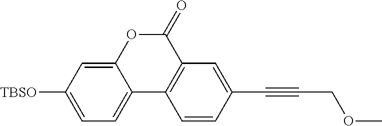 [SMILES: COCC#Cc1ccc2c(c1)c(=O)oc1cc(O[Si](C)(C)C(C)(C)C)ccc12]

To a solution of 8-bromo-3-((tert-butyldimethylsilyl)oxy)-6H-benzo[c]chromen-6-one (370 mg, 0.910 mmol, 1.0 eq.) in THF (3.04 mL) in a 20 mL Biotage MW vial was subsequently added 3-methoxyprop-1-yne (224 mg, 3.19 mmol, 3.5 eq.), Pd(PPh₃)₂Cl₂ (64 mg, 0.09 mmol, 0.1 eq.) and CuI (17 mg, 0.09 mmol, 0.1 eq.) and the reaction was degassed at r.t. with N₂ for 10 min. Triethylamine (277 mg, 2.74 mmol, 3.0 eq.) was added in one portion and the reaction mixture was put into a pre-heated oil-bath at 90° C. Upon full conversion of the starting material (as indicated by TLC) the reaction mixture was allowed to cool to r.t. and quenched with water and extracted with EtOAc (2×25 mL). The combined organic layers were dried over anhydrous Na₂SO₄ and concentrated under vacuum. The crude product was purified by MPLC (SiO₂, 40 g, EtOAc in Hex 0-40%) to afford 3-((tert-butyldimethylsilyl)oxy)-8-(3-methoxyprop-1-yn-1-yl)-6H-benzo[c]chromen-6-one (160 mg 44%) as a brownish solid. ¹H NMR (400 MHz, CDCl₃) δ 8.42 (d, J=1.8 Hz, 1H), 7.94 (d, J=8.4 Hz, 1H), 7.87 (d, J=8.4 Hz, 1H), 7.80 (dd, J=8.4, 1.8 Hz, 1H), 6.86-6.81 (m, 2H), 4.35 (s, 2H), 3.48 (s, 3H), 1.00 (s, 9H), 0.26 (s, 6H).

### Step 2: Synthesis of 3-hydroxy-8-(3-methoxyprop-1-yn-1-yl)-6H-benzo[c]chromen-6-one

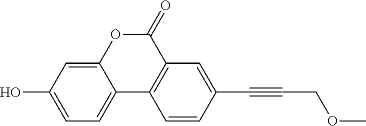 [SMILES: COCC#Cc1ccc2c(c1)c(=O)oc1cc(O)ccc12]

3-((tert-butyldimethylsilyl)oxy)-8-(3-methoxyprop-1-yn-1-yl)-6H-benzo[c]chromen-6-one (160 mg, 0.410 mmol, 1.0 eq.) was dissolved in MeOH (2 mL) and cooled to r.t. in an ice-bath and the resulting yellow solution was stirred for 10 min. Then KHF₂ (63 mg, 0.81 mmol, 2.0 eq.) was added in on portion and the reaction was stirred overnight. Upon complete consumption of the starting material (as indicated by TLC) the reaction mixture was filtered over a glass frit (Por.4) and the filter residue was washed with MeOH and dried under vacuum to afford 3-hydroxy-8-(3-methoxyprop-1-yn-1-yl)-6H-benzo[c]chromen-6-one (85 mg, 75%) as a pale brown solid. ¹H NMR (400 MHz, DMSO) δ 10.45 (s, 1H), 8.28 (d, J=8.5 Hz, 1H), 8.20-8.13 (m, 2H), 7.91 (dd, J=8.4, 1.9 Hz, 1H), 6.86 (dd, J=8.7, 2.4 Hz, 1H), 6.76 (d, J=2.4 Hz, 1H), 4.38 (s, 2H), 3.36 (s, 3H).

### Synthesis of 3-hydroxy-8-(3-hydroxy-3-methylbut-1-yn-1-yl)-6H-benzo[c]chromen-6-one (42)

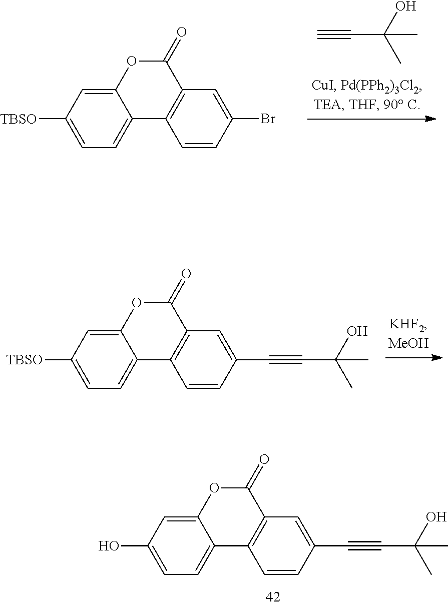

### Step 1: Synthesis of 3-((tert-butyldimethylsilyl)oxy)-8-(3-hydroxy-3-methylbut-1-yn-1-yl)-6H-benzo[c]chromen-6-one

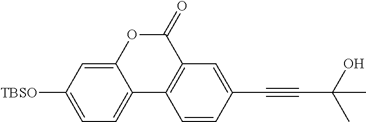 [SMILES: CC(C)(O)C#Cc1ccc2c(c1)c(=O)oc1cc(O[Si](C)(C)C(C)(C)C)ccc12]

To a solution of 8-bromo-3-((tert-butyldimethylsilyl)oxy)-6H-benzo[c]chromen-6-one (370 mg, 0.910 mmol, 1.0 eq.) in THF (3.0 mL) in a 20 mL Biotage MW vial was subsequently added 2-methylbut-3-yn-2-ol (269 mg, 3.19 mmol, 3.5 eq.), Pd(PPh₃)₂Cl₂ (64 mg, 0.090 mmol, 0.1 eq.) and CuI (17 mg, 0.090 mmol, 0.1 eq.) and the reaction was degassed at r.t. with N₂ for 10 min. Triethylamine (277 mg, 2.74 mmol, 3.0 eq.) was added in one portion and the reaction mixture was put into a pre-heated oil-bath at 90° C. Upon full conversion of the starting material (as indicated by TLC) the reaction mixture was allowed to cool to r.t. and quenched with water and extracted with EtOAc (2×25 mL). The combined organic layers were dried over anhydrous Na₂SO₄ and concentrated under vacuum. The crude product was purified by MPLC (SiO₂, 40 g, EtOAc in Hex 0-40%) to afford 3-((tert-butyldimethylsilyl)oxy)-8-(3-hydroxy-3-methylbut-1-yn-1-yl)-6H-benzo[c]chromen-6-one (233 mg, 0.570 mmol, 63%) as a yellowish solid. ¹H NMR (400 MHz, CDCl₃) δ 8.41 (d, J=1.8 Hz, 1H), 7.94 (d, J=8.4 Hz, 1H), 7.88 (d, J=8.3 Hz, 1H), 7.78 (dd, J=8.4, 1.9 Hz, 1H), 6.88-6.82 (m, 2H), 1.65 (s, 6H), 1.00 (s, 9H), 0.26 (s, 6H).

### Step 2: Synthesis of 3-hydroxy-8-(3-hydroxy-3-methylbut-1-yn-1-yl)-6H-benzo[c]chromen-6-one

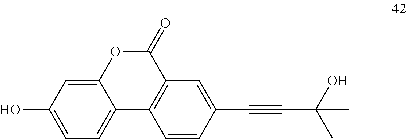 [SMILES: CC(C)(O)C#Cc1ccc2c(c1)c(=O)oc1cc(O)ccc12]

3-((tert-butyldimethylsilyl)oxy)-8-(3-hydroxy-3-methylbut-1-yn-1-yl)-6H-benzo[c]chromen-6-one (233 mg, 0.570 mmol, 1.0 eq.) was dissolved in MeOH (3 mL) and cooled to r.t. in an ice-bath and the resulting yellow solution was stirred for 10 min. Then KHF₂ (89 mg, 1.1 mmol, 2.0 eq.) was added in on portion and the reaction was stirred overnight. Upon complete consumption of the starting material (as indicated by TLC) the reaction mixture was filtered over a glass frit (Por.4) and the filter residue was washed with MeOH and dried under vacuum to afford 3-hydroxy-8-(3-hydroxy-3-methylbut-1-yn-1-yl)-6H-benzo[c]chromen-6-one (120 mg, 0.410 mmol, 72%) as a pale brown solid. ¹H NMR (400 MHz, DMSO) δ 10.44 (s, 1H), 8.25 (d, J=8.5 Hz, 1H), 8.16 (d, J=8.9 Hz, 1H), 8.10 (d, J=1.8 Hz, 1H), 7.83 (dd, J=8.4, 1.9 Hz, 1H), 6.86 (dd, J=8.8, 2.4 Hz, 1H), 6.76 (d, J=2.4 Hz, 1H), 5.54 (s, 1H), 3.32 (s, 6H).

### Synthesis of 3-hydroxy-8-((1-hydroxycyclobutyl)ethynyl)-6H-benzo[c]chromen-6-one (43)

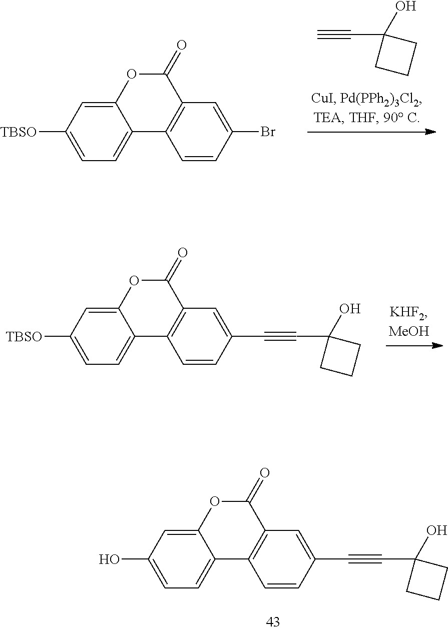

### Step 1: Synthesis of 3-((tert-butyldimethylsilyl)oxy)-8-((1-hydroxycyclobutyl)ethynyl)-6H-benzo[c]chromen-6-one

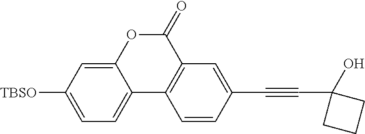 [SMILES: CC(C)(C)[Si](C)(C)Oc1ccc2c(c1)oc(=O)c1cc(C#CC3(O)CCC3)ccc12]

To a solution of 8-bromo-3-((tert-butyldimethylsilyl)oxy)-6H-benzo[c]chromen-6-one (250 mg, 0.620 mmol, 1.0 eq.) in THF (2.06 mL) in a 20 mL Biotage MW vial was subsequently added 1-ethynylcyclobutan-1-ol (208 mg, 2.16 mmol, 3.5 eq.), Pd(PPh₃)₂Cl₂ (43 mg, 0.060 mmol, 0.1 eq.) and CuI (12 mg, 0.060 mmol, 0.1 eq.) and the reaction was degassed at r.t. with N₂ for 10 min. Triethylamine (187 mg, 1.85 mmol, 3.00 eq.) was added in one portion and the reaction mixture was put into a pre-heated oil-bath at 90° C. Upon full conversion of the starting material (as indicated by TLC) the reaction mixture was allowed to cool to r.t., quenched with water and extracted with EtOAc (2×20 mL). The combined organic layers were dried over anhydrous Na₂SO₄ and concentrated under vacuum. The crude product was purified by MPLC (SiO₂, 40 g, EtOAc in Hex 0-40%) to afford 3-((tert-butyldimethylsilyl)oxy)-8-((1-hydroxycyclobutyl)ethynyl)-6H-benzo[c]chromen-6-one (195 mg, 75%) as a yellowish solid. ¹H NMR (400 MHz, CDCl₃) δ 8.38 (d, J=1.8 Hz, 1H), 7.90 (d, J=8.4 Hz, 1H), 7.85 (d, J=8.6 Hz, 1H), 7.76 (dd, J=8.4, 1.9 Hz, 1H), 6.86-6.78 (m, 2H), 2.61-2.52 (m, 2H), 2.36 (td, J=9.3, 2.8 Hz, 2H), 2.06-1.79 (m, 2H), 1.00 (s, 9H), 0.25 (s, 6H).

### Step 2: Synthesis of 3-hydroxy-8-((1-hydroxycyclobutyl)ethynyl)-6H-benzo[c]chromen-6-one

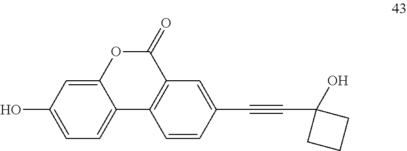 [SMILES: O=c1oc2cc(O)ccc2c2ccc(C#CC3(O)CCC3)cc12]

3-((tert-butyldimethylsilyl)oxy)-8-((1-hydroxycyclobutyl)ethynyl-6H-benzo[c]chromen-6-one (195 mg, 0.460 mmol, 1.0 eq.) was dissolved in MeOH (2 mL) and cooled to r.t. in an ice-bath and the resulting yellow solution was stirred for 10 min. Then KHF₂ (72 mg, 0.93 mmol, 2.0 eq.) was added in on portion and the reaction was stirred overnight. Upon complete consumption of the starting material (as indicated by TLC) the reaction mixture was filtered over a glass frit (Por.4) and the filter residue was washed with MeOH and dried under vacuum to afford 3-hydroxy-8-((1-hydroxycyclobutyl)ethynyl)-6H-benzo[c]chromen-6-one (100 mg, 70%) as a white solid. ¹H NMR (400 MHz, DMSO) δ 10.44 (s, 1H), 8.26 (d, J=8.4 Hz, 1H), 8.17 (d, J=8.9 Hz, 1H), 8.13 (d, J=1.8 Hz, 1H), 7.87 (dd, J=8.4, 1.9 Hz, 1H), 6.86 (dd, J=8.7, 2.4 Hz, 1H), 6.76 (d, J=2.4 Hz, 1H), 5.95 (s, 1H), 2.41 (ddd, J=9.2, 7.6, 4.4 Hz, 2H), 2.25 (td, J=9.3, 2.7 Hz, 2H), 1.84-1.76 (m, 2H).

### Synthesis of 3-hydroxy-9-(3-hydroxyprop-1-yn-1-yl)-6H-benzo[c]chromen-6-one (44)

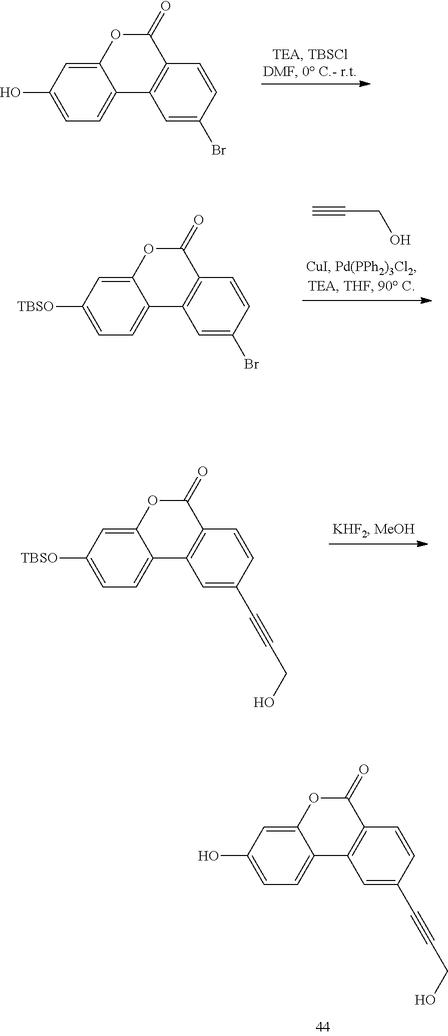

### Step 1: Synthesis of 9-bromo-3-((tert-butyldimethylsilyl)oxy)-6H-benzo[c]chromen-6-one

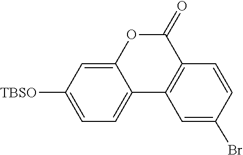 [SMILES: CC(C)(C)[Si](C)(C)Oc1ccc2c(c1)oc(=O)c1ccc(Br)cc12]

9-bromo-3-hydroxy-6H-benzo[c]chromen-6-one (610 mg, 2.10 mmol, 1.0 eq.) was suspended in DMF (10 mL) and triethylamine (636 mg, 6.29 mmol, 3.0 eq.) was added in one portion. The reaction mixture was cooled to 0° C. in an ice-bath and stirred at this temperature for 10 min. Subsequently, TBSCl (411 mg, 2.72 mmol, 1.3 eq.) was added in one portion and the reaction mixture was allowed to warm to r.t. and stirred for an additional 2 h. Upon full conversion of the starting material (as indicated by TLC) the reaction mixture was quenched with half-saturated aqueous sodium bicarbonate solution, extracted with ethyl acetate and the combined organic phases dried over anhydrous Na₂SO₄. The crude product was purified by MPLC (SiO₂, 80 g, EtOAc in Hex 0-15%) to afford 9-bromo-3-((tert-butyldimethylsilyl)oxy)-6H-benzo[c]chromen-6-one (566 mg, 67%) as a light brown solid. ¹H NMR (400 MHz, CDCl₃) δ 8.10 (d, J=8.4 Hz, 1H), 8.04 (d, J=1.8 Hz, 1H), 7.75 (d, J=8.5 Hz, 1H), 7.52 (dd, J=8.5, 1.8 Hz, 1H), 6.84-6.67 (m, 2H), 0.90 (s, 9H), 0.17 (s, 6H).

### Step 2: Synthesis of 3-((tert-butyldimethylsilyl)oxy)-9-(3-hydroxyprop-1-yn-1-yl)-6H-benzo[c]chromen-6-one

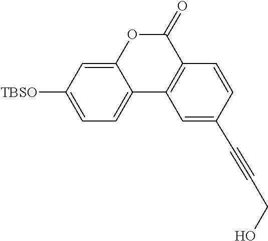 [SMILES: CC(C)(C)[Si](C)(C)Oc1ccc2c(c1)oc(=O)c1ccc(C#CCO)cc12]

To a solution of 9-bromo-3-((tert-butyldimethylsilyl)oxy)-6H-benzo[c]chromen-6-one (250 mg, 0.620 mmol, 1.0 eq.) in THF (2.0 mL) in a 20 mL Biotage MW vial was subsequently added propargyl alcohol (208 mg, 2.16 mmol, 3.5 eq.), Pd(PPh₃)₂Cl₂ (43 mg, 0.060 mmol, 0.1 eq.) and CuI (12 mg, 0.060 mmol, 0.1 eq.) and the reaction was sparged at r.t. with N₂ for 10 min. Triethylamine (187 mg, 1.85 mmol, 3.0 eq.) was added in one portion and the reaction mixture was put into a pre-heated oil-bath at 90° C. Upon full conversion of the starting material (as indicated by TLC) the reaction mixture was allowed to cool to r.t., quenched with water and extracted with EtOAc (2×20 mL). The combined organic layers were dried over anhydrous Na₂SO₄ and concentrated under vacuum. The crude product was purified by MPLC (SiO₂, 40 g, EtOAc in Hex 0-40%) to afford 3-((tert-butyldimethylsilyl)oxy)-9-(3-hydroxyprop-1-yn-1-yl)-6H-benzo[c]chromen-6-one (176 mg, 75%) as a yellowish solid. ¹H NMR (500 MHz, DMSO) δ 11.36 (s, 1H), 8.28 (s, 1H), 8.19 (dd, J=8.8, 1.6 Hz, 1H), 8.13 (d, J=8.2 Hz, 1H), 7.51 (d, J=8.2 Hz, 1H), 6.80 (dd, J=8.8, 2.4 Hz, 1H), 6.71 (d, J=2.4 Hz, 1H), 5.63 (d, J=124.9 Hz, 1H), 4.39 (s, 2H), 0.90 (s, 9H), 0.17 (s, 6H).

### Step 2: Synthesis of 3-hydroxy-9-(3-hydroxyprop-1-yn-1-yl)-6H-benzo[c]chromen-6-one

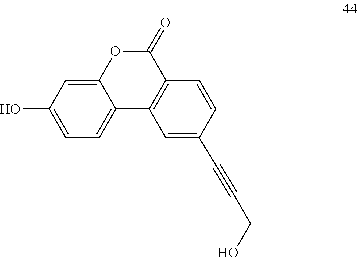 [SMILES: O=c1oc2cc(O)ccc2c2cc(C#CCO)ccc12]

3-((tert-butyldimethylsilyl)oxy)-9-(3-hydroxyprop-1-yn-1-yl)-6H-benzo[c]chromen-6-one (176 mg, 0.460 mmol, 1.0 eq.) was dissolved in MeOH (2 mL) and the resulting yellow solution was stirred for 10 min. Then KHF₂ (72 mg, 0.93 mmol, 2.0 eq.) was added in on portion and the reaction was stirred overnight. Upon complete consumption of the starting material (as indicated by TLC) the reaction mixture was filtered over a glass frit (Por.4) and the filter residue was washed with MeOH and dried under vacuum to yield 3-hydroxy-8-((1-hydroxycyclobutyl)ethynyl)-6H-benzo[c]chromen-6-one (90 mg, 0.34 mmol, 73%) as a white solid. MS (ESI+): m/z=267. ¹H NMR (500 MHz, DMSO) δ 11.36 (s, 1H), 8.28 (s, 1H), 8.19 (dd, J=8.8, 1.6 Hz, 1H), 8.13 (d, J=8.2 Hz, 1H), 7.51 (d, J=8.2 Hz, 1H), 6.80 (dd, J=8.8, 2.4 Hz, 1H), 6.71 (d, J=2.4 Hz, 1H), 5.63 (d, J=124.9 Hz, 1H), 4.39 (s, 2H).

### Synthesis of 3-((tert-butyldimethylsilyl)oxy)-8-(3-(4-methylpiperazin-1-yl)prop-1-yn-1-yl)-6H-benzo[c]chromen-6-one

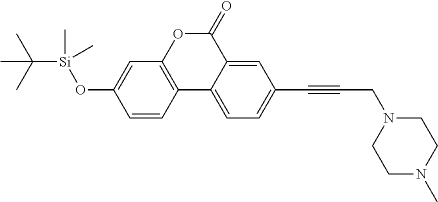 [SMILES: CN1CCN(CC#Cc2ccc3c(c2)c(=O)oc2cc(O[Si](C)(C)C(C)(C)C)ccc23)CC1]

Mesyl Chloride (0.037 mL, 0.47 mmol) was added to a solution of 3-((tert-butyldimethylsilyl)oxy)-8-(3-hydroxyprop-1-yn-1-yl)-6H-benzo[c]chromen-6-one (140 mg, 0.360 mmol) and NEt₃ (0.150 ml, 1.10 mmol) in THF (5 mL) at 0° C. and the reaction mixture was stirred at rt for 1 h. TLC showed complete conversion of the starting material. N-methylpiperazine (111 mg, 1.10 mmol) was added and the mixture was heated at 60° C. overnight. A saturated solution of Ammonium chloride was added and the aqueous layer was extracted with EtOAc 3 times. The combined organic layers were dried over sodium sulfate and concentrated under vacuum. The crude product was purified by MPLC (SiO₂, MeOH/DCM 0% to 20%) to afford 3-((tert-butyldimethylsilyl)oxy)-8-(3-(4-methylpiperazin-1-yl)prop-1-yn-1-yl)-6H-benzo[c]chromen-6-one, which was used without further purification in the next step.

### Synthesis of 3-((tert-butyldimethylsilyl)oxy)-8-(3-(4-methylpiperazin-1-yl)propyl)-6H-benzo[c]chromen-6-one

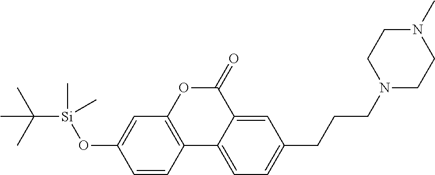 [SMILES: CN1CCN(CCCc2ccc3c(c2)c(=O)oc2cc(O[Si](C)(C)C(C)(C)C)ccc23)CC1]

A suspension 3-((tert-butyldimethylsilyl)oxy)-8-(3-(4-methylpiperazin-1-yl)prop-1-yn-1-yl)-6H-benzo[c]chromen-6-one 10 (67 mg, 0.14 mmol) and Pd(OH)₂/C (20 mg, 0.030 mmol) in MeOH (5 ml) was hydrogenated under atmospheric pressure overnight. The reaction mixture was filtered over a pad of celite and the solvent was evaporated concentrate under vacuum to afford 3-((tert-butyldimethylsilyl)oxy)-8-(3-(4-methylpiperazin-1-yl)propyl)-6H-benzo[c]chromen-6-one (64 mg, 95%) as yellowish oil, which was used without further purification in the next step.

### Synthesis of 3-hydroxy-8-(3-(4-methylpiperazin-1-yl)propyl)-6H-benzo[c]chromen-6-one (45)

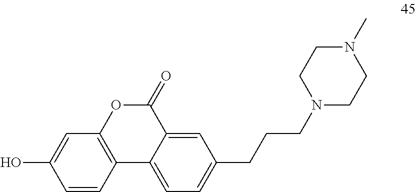 [SMILES: CN1CCN(CCCc2ccc3c(c2)c(=O)oc2cc(O)ccc23)CC1]

45 was prepared starting from 3-((tert-butyldimethylsilyl)oxy)-8-(3-(4-methylpiperazin-1-yl)propyl)-6H-benzo[c]chromen-6-one (65 mg, 0.14 mmol) and KHF₂ (22 mg, 0.28 mmol) to afford after purification by MPLC (SiO₂, MeOH/DCM 5% to 30%) 3-hydroxy-8-(3-(4-methylpiperazin-1-yl)propyl)-6H-benzo[c]chromen-6-one (36 mg, 73%) as a yellowish solid. R_{f}=0.4 (MeOH/DCM 30/70). ¹H NMR (400 MHz, CDCl₃) δ 8.04 (s, 1H), 7.73 (d, J=8.5 Hz, 1H), 7.66 (d, J=8.8 Hz, 1H), 7.51 (d, J=6.5 Hz, 1H), 6.61 (d, J=9.1 Hz, 1H), 6.53 (s, 1H), 2.86-2.48 (m, 12H), 2.39 (s, 3H), 1.99 (s, 2H).

### Synthesis of 3-((tert-butyldimethylsilyl)oxy)-8-(3-morpholinoprop-1-yn-1-yl)-6H-benzo[c]chromen-6-one

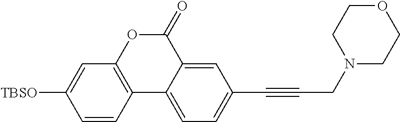 [SMILES: CC(C)(C)[Si](C)(C)Oc1ccc2c(c1)oc(=O)c1cc(C#CCN3CCOCC3)ccc12]

Mesyl Chloride (0.04 ml, 0.51 mmol) was added to a solution of 3-((tert-butyldimethylsilyl)oxy)-8-(3-hydroxyprop-1-yn-1-yl)-6H-benzo[c]chromen-6-one (150 mg, 0.39 mmol) and NEt₃ (0.160 ml, 1.18 mmol) in THF (5 mL) at 0° C. and the reaction mixture was stirred at rt for 1 h. TLC showed complete conversion of the starting material. Morpholine (0.100 ml, 1.18 mmol) was added and the mixture was heated at 60° C. overnight. A saturated solution of Ammonium chloride was added and the reaction mixture was extracted with EtOAc 3 times. The combined organic layers were dried over sodium sulfate and concentrated under vacuum. The crude product was purified by MPLC (SiO₂, MeOH/DCM 0% to 20%) to afford 3-((tert-butyldimethylsilyl)oxy)-8-(3-morpholinoprop-1-yn-1-yl)-6H-benzo[c]chromen-6-one (101 mg, 57%), which was used without further purification in the next step.

### Synthesis of 3-((tert-butyldimethylsilyl)oxy)-8-(3-morpholinopropyl)-6H-benzo[c]chromen-6-one

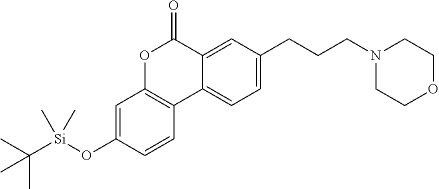 [SMILES: CC(C)(C)[Si](C)(C)Oc1ccc2c(c1)oc(=O)c1cc(CCCN3CCOCC3)ccc12]

3-(3-((tert-butyldimethylsilyl)oxy)-6-oxo-6H-benzo[c]chromen-8-yl)prop-2-yn-1-yl methanesulfonate OTBS-morpholine (100 mg, 0.220 mmol) and Pd(OH)₂/C (31 mg, 0.22 mmol) in MeOH (5 ml) was hydrogenated under atmospheric pressure overnight. The reaction mixture was filtered over a pad of celite and the solvent was concentrated under vacuum to afford 3-((tert-butyldimethylsilyl)oxy)-8-(3-morpholinopropyl)-6H-benzo[c]chromen-6-one (70 mg, 69%) as a yellowish oil. ¹H NMR (400 MHz, CDCl₃) δ 8.16 (d, J=1.9 Hz, 1H), 7.91 (d, J=8.3 Hz, 1H), 7.87 (d, J=9.4 Hz, 1H), 7.61 (dd, J=8.2, 2.0 Hz, 1H), 6.82 (h, J=2.4 Hz, 2H), 3.71 (t, J=4.7 Hz, 4H), 2.76 (d, J=7.8 Hz, 2H), 2.43 (t, J=4.6 Hz, 4H), 2.36 (dd, J=8.4, 6.4 Hz, 2H), 1.87 (h, J=7.4, 6.8 Hz, 2H), 0.99 (s, 9H), 0.24 (s, 6H).

### Synthesis of 3-hydroxy-8-(3-morpholinopropyl)-6H-benzo[c]chromen-6-one (46)

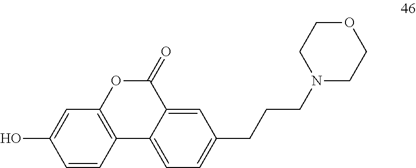 [SMILES: O=c1oc2cc(O)ccc2c2ccc(CCCN3CCOCC3)cc12]

46 was prepared starting from 3-((tert-butyldimethylsilyl)oxy)-8-(3-morpholinopropyl)-6H-benzo[c]chromen-6-one (70 mg, 0.15 mmol) and KHF₂ (24 mg, 0.31 mmol) to afford after purification by MPLC (SiO₂, MeOH/DCM 5% to 30%) 3-hydroxy-8-(3-morpholinopropyl)-6H-benzo[c]chromen-6-one (70 mg, 69%) as a yellowish solid. R_{f}=0.4 (MeOH/DCM 30/70). ¹H NMR (400 MHz, DMSO) δ 10.31 (s, 1H), 8.18 (d, J=8.3 Hz, 1H), 8.13 (d, J=8.9 Hz, 1H), 8.01 (d, J=1.9 Hz, 1H), 7.75 (dd, J=8.3, 2.0 Hz, 1H), 6.83 (dd, J=8.7, 2.4 Hz, 1H), 6.74 (d, J=2.4 Hz, 1H), 3.57 (t, J=4.7 Hz, 4H), 2.74 (t, J=7.6 Hz, 2H), 2.35-2.31 (m, 4H), 2.28 (t, J=7.2 Hz, 2H), 1.78 (p, J=7.4 Hz, 2H).

### Synthesis of 3-((tert-butyldimethylsilyl)oxy)-8-(3-(piperidin-1-yl)prop-1-yn-1-yl)-6H-benzo[c]chromen-6-one

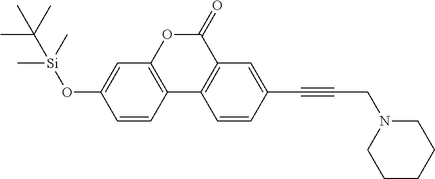 [SMILES: CC(C)(C)[Si](C)(C)Oc1ccc2c(c1)oc(=O)c1cc(C#CCN3CCCCC3)ccc12]

Mesyl Chloride (0.0980 ml, 1.26 mmol) was added to a solution of 3-((tert-butyldimethylsilyl)oxy)-8-(3-hydroxyprop-1-yn-1-yl)-6H-benzo[c]chromen-6-one (240 mg, 0.630 mmol) and NEt₃ (0.260 ml, 1.89 mmol) in THF (10 mL) at 0° C. and the reaction mixture was stirred at rt for 1 h. TLC showed complete conversion of the starting material. Piperidine (0.081 ml, 0.82 mmol) was added and the mixture was heated at 60° C. overnight. A saturated solution of Ammonium chloride was added and the reaction mixture was extracted with EtOAc 3 times. The combined organic layers were dried over sodium sulfate and concentrated under vacuum. The crude product was purified by MPLC (SiO₂, MeOH/DCM 0% to 20%) to afford 3-((tert-butyldimethylsilyl)oxy)-8-(3-(piperidin-1-yl)prop-1-yn-1-yl)-6H-benzo[c]chromen-6-one (66 mg, 23%). ¹H NMR (400 MHz, CDCl₃) δ 8.41 (d, J=1.8 Hz, 1H), 7.93 (d, J=8.4 Hz, 1H), 7.88 (d, J=8.3 Hz, 1H), 7.79 (dd, J=8.3, 1.8 Hz, 1H), 6.85 (d, J=8.2 Hz, 2H), 3.53 (s, 2H), 2.61 (s, 4H), 1.70-1.45 (m, 6H), 1.00 (s, 9H), 0.26 (s, 6H).

### Synthesis of 3-hydroxy-8-(3-(piperidin-1-yl)prop-1-yn-1-yl)-6H-benzo[c]chromen-6-one (47)

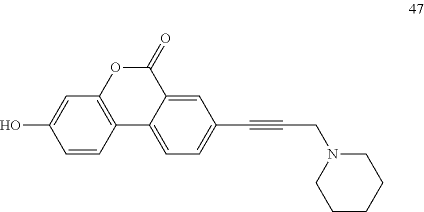 [SMILES: O=c1oc2cc(O)ccc2c2ccc(C#CCN3CCCCC3)cc12]

47 was prepared starting from 3-((tert-butyldimethylsilyl)oxy)-8-(3-(piperidin-1-yl)prop-1-yn-1-yl)-6H-benzo[c]chromen-6-one (60 mg, 0.13 mmol) and KHF₂ (21 mg, 0.27 mmol) to afford after purification by MPLC (SiO₂, EtOAc/cyclohexane 0% to 80%) to afford 3-hydroxy-8-(3-(piperidin-1-yl)prop-1-yn-1-yl)-6H-benzo[c]chromen-6-one (37 mg, 83%) as a yellowish solid. R_{f}=0.4 (EtOAc/hexane 40%). ¹H NMR (400 MHz, CDCl₃) δ 7.70-7.62 (m, 2H), 7.59 (d, J=1.7 Hz, 1H), 7.54 (dd, J=8.4, 1.8 Hz, 1H), 6.82 (dd, J=8.7, 2.4 Hz, 1H), 6.55 (d, J=2.4 Hz, 1H), 3.36 (s, 2H), 2.77 (s, 4H), 1.80 (q, J=5.7 Hz, 4H), 1.58 (b, 2H).

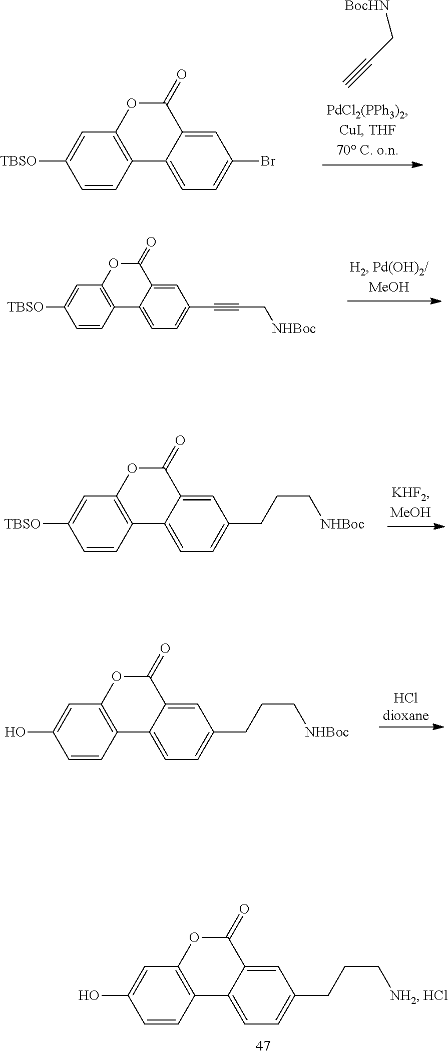

### Synthesis of tert-butyl (3-(3-((tert-butyldimethylsilyl)oxy)-6-oxo-6H-benzo[c]chromen-8-yl)prop-2-yn-1-yl)carbamate

To a well degassed solution of Pd(PPh₃)₂Cl₂ (41.8 mg, 0.059 mmol, 0.1 eq) and CuI (11.3 mg, 0.059 mmol, 0.1 eq) in THF (10 ml) and 8-bromo-3-((dimethyl(tert-butyl)silyl)oxy)-6H-benzo[c]chromen-6-one (250 mg, 0.590 mmol) and Prop-2-ynyl-carbamic acid tert-butyl ester (277 mg, 1.79 mmol, 3.0 eq) was added NEt₃ (0.330 ml, 2.38 mmol, 4.0 eq) and the mixture was heated at 70° C. overnight. The reaction mixture was diluted with a saturated solution of NH₄Cl, and extracted with EtOAc. The organic layers was dried over sodium sulfate and concentrated under vacuum. The crude product was purified by MPLC (SiO₂, EtOAc/hexane 0% to 20%) to afford tert-butyl (3-(3-((tert-butyldimethylsilyl)oxy)-6-oxo-6H-benzo[c]chromen-8-yl)prop-2-yn-1-yl)carbamate (190 mg, 0.39 mmol, 66%) as a yellowish foam. R_{f}=0.4 (EtOAc/hexane 20%). ¹H NMR (400 MHz, CDCl₃) δ 8.39 (d, J=1.7 Hz, 1H), 7.93 (d, J=8.4 Hz, 1H), 7.87 (dd, J=8.5, 0.8 Hz, 1H), 7.77 (dd, J=8.3, 1.8 Hz, 1H), 6.89-6.81 (m, 2H), 4.79 (s, 1H), 4.19 (d, J=5.6 Hz, 2H), 1.48 (s, 9H), 1.00 (s, 9H), 0.26 (s, 6H).

### Synthesis of tert-butyl (3-(3-((tert-butyldimethylsilyl)oxy)-6-oxo-6H-benzo[c]chromen-8-yl)propyl)carbamate

A suspension of tert-butyl (3-(3-((tert-butyldimethylsilyl)oxy)-6-oxo-6H-benzo[c]chromen-8-yl)prop-2-yn-1-yl)carbamate (190 mg, 0.390 mmol) and Pd(OH)₂/C 20% (56 mg, 0.79 mmol) was hydrogenated under atmospheric pressure in methanol, and stirred overnight. The reaction mixture was filtered over a pad of celite, and the solvent was evaporated under vacuum. The crude product was purified by MPLC (SiO₂, EtOAc/cyclohexane 0% to 20%) to afford tert-butyl (3-(3-((tert-butyldimethylsilyl)oxy)-6-oxo-6H-benzo[c]chromen-8-yl)propyl)carbamate (175 mg, 91%) as a yellowish oil. R_{f}=0.4 (EtOAc/hexane 20%). ¹H NMR (400 MHz, CDCl₃) δ 8.17 (d, J=1.9 Hz, 1H), 7.93 (d, J=8.3 Hz, 1H), 7.88 (d, J=9.3 Hz, 1H), 7.62 (dd, J=8.2, 2.0 Hz, 1H), 6.84 (dq, J=4.5, 2.4 Hz, 2H), 4.57 (s, 1H), 3.18 (d, J=7.0 Hz, 2H), 2.89-2.71 (m, 2H), 1.88 (p, J=7.3 Hz, 2H), 1.45 (s, 9H), 1.00 (s, 9H), 0.26 (s, 6H).

### Synthesis of tert-butyl (3-(3-hydroxy-6-oxo-6H-benzo[c]chromen-8-yl)propyl)carbamate (48)

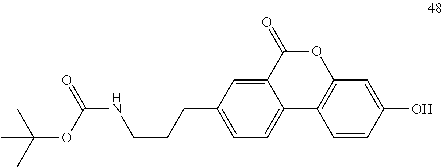 [SMILES: CC(C)(C)OC(=O)NCCCc1ccc2c(c1)c(=O)oc1cc(O)ccc12]

48 was prepared starting from tert-butyl (3-(3-((tert-butyldimethylsilyl)oxy)-6-oxo-6H-benzo[c]chromen-8-yl)propyl)carbamate (170 mg, 0.350 mmol) and KHF₂ (55 mg, 0.70 mmol) to afford after purification by MPLC (SiO₂, EtOAc/cyclohexane 0% to 20%) tert-butyl (3-(3-hydroxy-6-oxo-6H-benzo[c]chromen-8-yl)propyl)carbamate (108 mg, 0.290 mmol, 83%) as a white solid. R_{f}=0.4 (EtOAc/hexane 20/100). ¹H NMR (400 MHz, DMSO) δ 10.27 (s, 1H), 8.15 (dd, J=21.4, 8.5 Hz, 2H), 8.00 (d, J=1.9 Hz, 1H), 7.73 (dd, J=8.3, 2.0 Hz, 1H), 6.87 (t, J=5.4 Hz, 1H), 6.82 (dd, J=8.7, 2.4 Hz, 1H), 6.73 (d, J=2.3 Hz, 1H), 2.94 (q, J=6.6 Hz, 2H), 2.70 (t, J=7.6 Hz, 2H), 1.72 (p, J=7.3 Hz, 2H), 1.36 (s, 9H).

### Synthesis of 8-(3-aminopropyl)-3-hydroxy-6H-benzo[c]chromen-6-one hydrochloride (49)

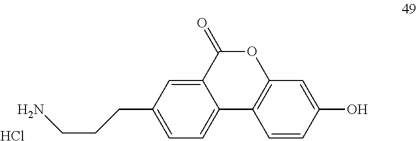 [SMILES: Cl ; NCCCc1ccc2c(c1)c(=O)oc1cc(O)ccc12]

HCl (4M in Dioxane, 1.35 mL, 5.4 mmol) was added to a solution of tert-butyl (3-(3-hydroxy-6-oxo-6H-benzo[c]chromen-8-yl)propyl)carbamate (100 mg, 0.270 mmol) in dioxane (0.5 ml) at r.t. and the reaction mixture was stirred at r.t. overnight, and a precipitate was formed. The solvent was concentrated under vacuum and the crude product was triturated in Et₂O, filtered, and dried to afford (3-aminopropyl)-3-hydroxy-6H-benzo[c]chromen-6-one hydrochloride (70 mg, 86%) as a white solid. MS (ESI+): m/z=270. ¹H NMR (400 MHz, DMSO) δ 10.36 (s, 1H), 8.22 (d, J=8.3 Hz, 1H), 8.14 (d, J=8.8 Hz, 1H), 8.05 (d, J=1.9 Hz, 1H), 7.83 (s, 3H), 7.76 (dd, J=8.3, 2.0 Hz, 1H), 6.85 (dd, J=8.7, 2.4 Hz, 1H), 6.76 (dd, J=2.4, 1.2 Hz, 1H), 2.81 (q, J=7.7, 6.4 Hz, 4H), 1.98-1.85 (m, 2H).

### H) Spirocycle (oxetane & azetidine) “A” ring analogies

### Synthesis of spiro[benzo[c]chromene-6,3′-oxetane]-3,8-diol (50)

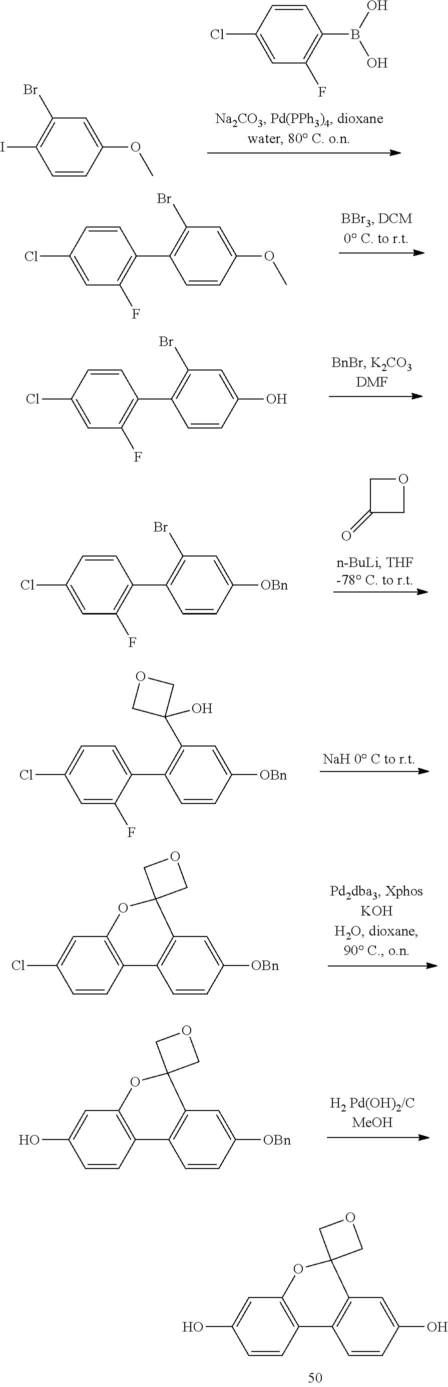

### Step 1: Synthesis of 2-bromo-4′-chloro-2′-fluoro-4-methoxy-1,1′-biphenyl

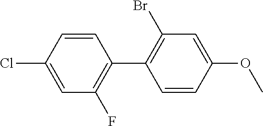 [SMILES: COc1ccc(-c2ccc(Cl)cc2F)c(Br)c1]

2-bromo-1-iodo-4-methoxybenzene (4.00 g, 12.8 mmol) and (4-chloro-2-fluorophenyl) boronic acid (1.01 g, 23.0 mmol) were dissolved in Dioxane (80 ml). Tetrakis(triphenylphosphine)palladium(0) (738 mg, 0.640 mmol) was added followed by a solution of Na₂CO₃ (2.70 g, 25.6 mmol) and the reaction mixture was heated at 80° C. overnight. The reaction mixture was diluted with a saturated solution of sodium carbonate, and extracted with EtOAc twice. The combined organic layers were dried over sodium sulfate, and concentrated in vacuum. The crude product was purified by MPLC (SiO₂, 0 to 8% DCM/cyclohexane) to afford 2-bromo-4′-chloro-2′-fluoro-4-methoxy-1,1′-biphenyl (1.80 g, 45%) as a colorless oil. R_{f}=0.2 (DCM/cyclohexane 3%). ¹H NMR (400 MHz, CDCl3) δ 7.24-7.14 (m, 5H), 6.92 (dd, J=8.5, 2.6 Hz, 1H), 3.84 (s, 3H).

### Step 2: Synthesis of 2-bromo-4′-chloro-2′-fluoro-[1,1′-biphenyl]-4-ol

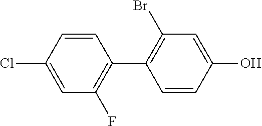 [SMILES: Oc1ccc(-c2ccc(Cl)cc2F)c(Br)c1]

BBr₃ (1M in DCM, 6.97 ml, 6.97 mmol) was added at 0° C. to a solution of 2-bromo-4′-chloro-2′-fluoro-4-methoxy-1,1′-biphenyl (1.10 g, 3.48 mmol) in DCM (5 mL) and the reaction mixture was allowed to warm to r.t. overnight. Methanol (10 ml) was added at 0° C. and the solvent was evaporated under vacuum. The crude product was diluted with a saturated solution of sodium bicarbonate and extracted with EtOAc. The combined organic layers were dried over sodium sulfate and concentrate under vacuum to afford 2-bromo-4′-chloro-2′-fluoro-[1,1′-biphenyl]-4-ol (1.10 g), which was used in the next step without further purification. ¹H NMR (400 MHz, CDCl₃) δ 7.23-7.01 (m, 5H), 6.79 (dd, J=8.4, 2.6 Hz, 1H).

### Step 3: Synthesis of 4-(benzyloxy)-2-bromo-4′-chloro-2′-fluoro-1,1′-biphenyl

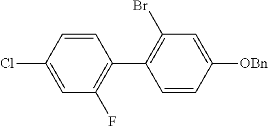 [SMILES: Fc1cc(Cl)ccc1-c1ccc(OCc2ccccc2)cc1Br]

Benzyl bromide (0.470 ml, 3.98 mmol) was added to a solution of 2-bromo-4′-chloro-2′-fluoro-[1,1′-biphenyl]-4-ol (1.00 g, 3.31 mmol) and potassium carbonate (0.916 g, 6.63 mmol) in ACN (10 ml) and the mixture was heated at 60° C. overnight. The crude was cooled to room temperature and extracted with Ethyl acetate from bicarbonate saturated solution. The combined organic layers were dried over sodium sulfate and concentrated under vacuum. The crude product was purified by MPLC (25 g silica cartridge, EtOAc/cyclohexane 0% to 10%) to afford 4-(benzyloxy)-2-bromo-4′-chloro-2′-fluoro-1,1′-biphenyl (1.10 g, 85%) as colorless oil. ¹H NMR (400 MHz, CDCl₃) δ 7.48-7.35 (m, 5H), 7.32 (d, J=2.6 Hz, 1H), 7.23-7.15 (m, 4H), 6.99 (dd, J=8.5, 2.6 Hz, 1H), 5.09 (s, 2H).

### Step 4: Synthesis of 3-(4-(benzyloxy)-4′-chloro-2′-fluoro-[1,1′-biphenyl]-2-yl)oxetan-3-ol

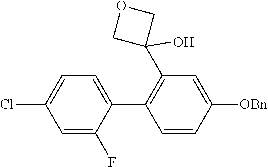 [SMILES: OC1(c2cc(OCc3ccccc3)ccc2-c2ccc(Cl)cc2F)COC1]

nBuLi (1.6M in hexane, 2.58 ml, 4.13 mmol) was added dropwise at −78° c. to a solution of 4-(benzyloxy)-2-bromo-4′-chloro-2′-fluoro-1,1′-biphenyl (900 mg, 2.29 mmol) in dry THF (8 ml). The red pale solution was stirred at −78° C. for 45 min then a solution of oxetan-3-one (662 mg, 9.19 mmol) was added dropwise and the reaction was allowed to warm to room temperature over 5 h. The reaction mixture was quenched with NH₄Cl saturated solution and extracted with Ethyl acetate. The organic layers were dried overs sodium sulfate. The crude product was purified by MPLC (25 g silica cartridge, EtOAc/cyclohexane 0% to 50%) to afford 3-(4-(benzyloxy)-4′-chloro-2′-fluoro-[1,1′-biphenyl]-2-yl)oxetan-3-ol (383 mg, 85%) as colorless oil. R_{f}=0.3 (EtOAc/hexane 50/50). ¹H NMR (400 MHz, CDCl3) δ 7.55-7.28 (m, 6H), 7.19-7.14 (m, 3H), 7.00 (dd, J=8.5, 2.6 Hz, 1H), 6.85 (d, J=2.6 Hz, 1H), 5.11 (s, 2H), 4.82 (s, 2H), 4.36 (s, 2H), 2.77 (s, 1H).

### Step 5: Synthesis of 8-(benzyloxy)-3-chlorospiro[benzo[c]chromene-6,3′-oxetane

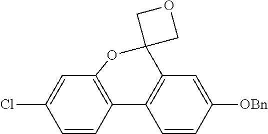 [SMILES: Clc1ccc2c(c1)OC1(COC1)c1cc(OCc3ccccc3)ccc1-2]

NaH (70.5 mg, 1.76 mmol, 60% dispersion in mineral oil) was added at 0° c. to a solution of 3-(4-(benzyloxy)-4′-chloro-2′-fluoro-[1,1′-biphenyl]-2-yl)oxetan-3-ol (377 mg, 0.980 mmol) in DMF 4 ml and the reaction was allowed to warm to room temperature overnight. The crude was extracted with 1/2 saturated solution of bicarbonate and ethyl acetate. The organic phase was dried over sodium sulfate and evaporated under vacuum. The crude product was purified by MPLC (25 g silica cartridge, EtOAc/cyclohexane 0% to 5%) to afford 8-(benzyloxy)-3-chlorospiro[benzo[c]chromene-6,3′-oxetane] (290 mg, 81%) as a yellow solid. R_{f}=0.3 (EtOAc/hexane 10%). ¹H NMR (400 MHz, CDCl₃) δ 7.63 (d, J=8.7 Hz, 1H), 7.54 (d, J=8.3 Hz, 1H), 7.49-7.35 (m, 5H), 7.32 (d, J=2.5 Hz, 1H), 7.08 (d, J=2.1 Hz, 1H), 7.04 (dd, J=8.7, 2.6 Hz, 1H), 7.01 (dd, J=8.3, 2.1 Hz, 1H), 5.17 (s, 2H), 5.08-5.01 (m, 2H), 4.90-4.78 (m, 2H).

### Step 6: Synthesis of 8-(benzyloxy)spiro[benzo[c]chromene-63′-oxetan]-3-ol

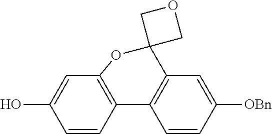 [SMILES: Oc1ccc2c(c1)OC1(COC1)c1cc(OCc3ccccc3)ccc1-2]

t-BuXPhos (9 mg, 0.020 mmol) was added to a suspension of Pd₂dba₃ (2.3 mg, 0.099 mmol) in Dioxane (1 ml), degassed and stirred for 5 minutes. 8-(benzyloxy)-3-chlorospiro[benzo[c]chromene-6,3′-oxetane] (45 mg, 0.12 mmol) was added followed by a solution of KOH (15 mg, 0.27 mmol) in water (0.3 ml) at rt and the mixture was heated at 90° C. overnight. Water was added and the mixture was extracted with EtOAc 3 times and the combined organic layers was dried over sodium sulfate, filtered and evaporated under vacuum. The crude product was purified by MPLC (25 g silica cartridge, EtOAc/cyclohexane 0% to 30%) to afford 8-(benzyloxy)spiro[benzo[c]chromene-6,3′-oxetan]-3-ol (30 mg, 0.87 mmol, 70%) as a white solid. R_{f}=0.3 (EtOAc/hexane 20%). MS (ESI+): m/z=347. ¹H NMR (400 MHz, DMSO) δ 9.72 (s, 1H), 7.69 (d, J=8.7 Hz, 1H), 7.60 (d, J=8.3 Hz, 1H), 7.53-7.36 (m, 6H), 7.09 (dd, J=8.6, 2.6 Hz, 1H), 6.56-6.43 (m, 2H), 5.21 (s, 2H), 4.86-4.80 (m, 4H).

### Step 7: Synthesis spiro[benzo[c]chromene-6,3′-oxetane]-3,8-diol

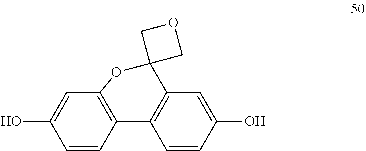 [SMILES: Oc1ccc2c(c1)OC1(COC1)c1cc(O)ccc1-2]

A suspension of 8-(benzyloxy)spiro[benzo[c]chromene-6,3′-oxetan]-3-ol (40 mg, 0.12 mmol) and Pd(OH)₂/C (16 mg, 0.23 mmol) in methanol (4 ml) was hydrogenated under atmospheric pressure o.n. The reaction mixture was filtered over a pad of celite the solvent was evaporated and the product further purified by filtration over a pad of silica using DCM/methanol 10% to afford spiro[benzo[c]chromene-6,3′-oxetane]-3,8-diol (23 mg, 0.09 mmol, 78%) as a light yellow solid. MS (ESI+): m/z=257. ¹H NMR (400 MHz, DMSO) δ 9.66 (d, J=19.6 Hz, 2H), 7.57 (d, J=8.5 Hz, 1H), 7.54 (d, J=8.4 Hz, 1H), 7.09 (d, J=2.4 Hz, 1H), 6.84 (dd, J=8.4, 2.4 Hz, 1H), 6.51-6.44 (m, 2H), 4.83 (d, J=7.3 Hz, 2H), 4.74 (d, J=7.2 Hz, 2H).

### Synthesis of spiro[azetidine-3,6′-benzo[c]chromene]-3′,8′-diol (51)

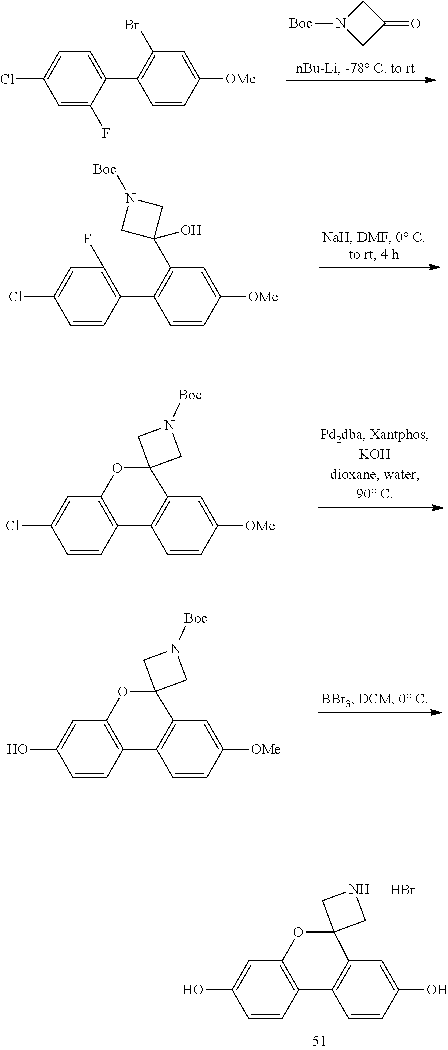

### Step 1: Synthesis of tert-butyl 3-(4′-chloro-2′-fluoro-4-methoxy-[1,1′-biphenyl]-2-yl)-3-hydroxyazetidine-1-carboxylate

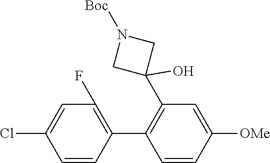 [SMILES: COc1ccc(-c2ccc(Cl)cc2F)c(C2(O)CN(C(=O)OC(C)(C)C)C2)c1]

nBuLi (1.6M in hexane, 2.69 ml, 4.31 mmol) was added dropwise at −78° c. to a solution of 4-(benzyloxy)-2-bromo-4′-chloro-2′-fluoro-1,1′-biphenyl (900 mg, 2.29 mmol) in dry THF (8 ml). The red pale solution was stirred at −78° C. for 45 min then a solution of tert-butyl 3-oxoazetidine-1-carboxylate (1.84 g, 10.8 mmol) in dry THF (5 ml) was added dropwise and the reaction was allowed to warm to room temperature over 5 hours. The reaction mixture was quenched with NH₄Cl saturated solution and extracted with Ethyl acetate. The organic phases were dried overs sodium sulfate. The crude product was purified by MPLC (80 g silica cartridge, EtOAc/cyclohexane 0% to 50%) to afford tert-butyl 3-(4′-chloro-2′-fluoro-4-methoxy-[1,1′-biphenyl]-2-yl)-3-hydroxyazetidine-1-carboxylate (400 mg, 36%) as a mixture of two compounds as a colorless oil. R_{f}=0.3 (EtOAc/hexane 50/50). ¹H NMR (400 MHz, CDCl₃) δ 7.32 (t, J=8.2 Hz, 1H), 7.21-7.12 (m, 3H), 6.93 (dd, J=8.5, 2.7 Hz, 1H), 6.86 (d, J=2.6 Hz, 1H), 4.18-3.97 (m, 1H), 3.95-3.87 (m, 1H), 3.86 (s, 3H), 3.73 (s, 2H), 2.70 (d, J=14.5 Hz, 1H), 1.39 (s, 9H).

### Step 2: Synthesis of tert-butyl 3′-chloro-8′-methoxyspiro[azetidine-3,6′-benzo[c]chromene]-1-carboxylate

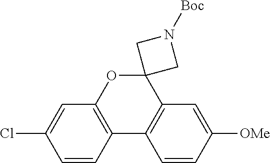 [SMILES: COc1ccc2c(c1)C1(CN(C(=O)OC(C)(C)C)C1)Oc1cc(Cl)ccc1-2]

NaH (12 mg, 0.30 mmol) was added at 0° C. to a solution of tert-butyl 3-(4′-chloro-2′-fluoro-4-methoxy-[1,1′-biphenyl]-2-yl)-3-hydroxyazetidine-1-carboxylate (67 mg, 0.16 mmol) in DMF 3 ml and the reaction mixture was stirred for 3 h. NH4Cl saturated solution was added and the aqueous phase was extracted twice with Ethyl acetate. The combined organic phases were dried over sodium sulfate, filtered and concentrated under vacuum. The crude was purified by MPLC (EtOAc/cyclohexane 0% to 8%) to give tert-butyl 3′-chloro-8′-methoxyspiro[azetidine-3,6′-benzo[c]chromene]-1-carboxylate (30 mg, 47%) as a yellowish solid. ¹H NMR (400 MHz, CDCl₃) δ 7.63 (d, J=8.5 Hz, 1H), 7.55 (d, J=8.3 Hz, 1H), 7.09-6.93 (m, 4H), 4.31 (d, J=9.5 Hz, 2H), 4.19 (s, 2H), 3.88 (s, 3H), 1.47 (s, 9H).

### Step 3: Synthesis of tert-butyl 3′-hydroxy-8′-methoxyspiro[azetidine-3,6′-benzo[c]chromene]-1-carboxylate

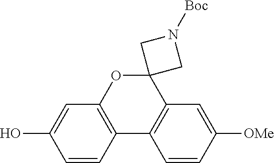 [SMILES: COc1ccc2c(c1)C1(CN(C(=O)OC(C)(C)C)C1)Oc1cc(O)ccc1-2]

3′-chloro-8′-methoxyspiro[azetidine-3,6′-benzo[c]chromene]-1-carboxylate (155 mg, 0.400 mmol, 1.0 eq.) was dissolved in 1,4-dioxane (1.5 mL) and Pd₂dba₃ (9 mg, 0.04 mmol, 0.1 eq.) as well as tBuXPhos (38 mg, 0.080 mmol, 0.2 eq.) were added to the solution. Following the mixture was degassed using a N₂ balloon for 10 min. Subsequently a solution of KOH (67 mg, 1.2 mmol, 3.0 eq.) in water (0.3 mL) was added in on portion before putting the reaction mixture in a pre-heated oil-bath at 90° C. Stirring was continued overnight and then the reaction was allowed to cool to r.t., quenched with water, the aq. phase extracted with ethyl acetate (3×10 mL) and the combined organic layers dried over Na₂SO₄ and concentrated under reduced pressure. The crude material was purified by flash column chromatography (SiO₂, 20 g, EtOAc in Hex 0-30%) to yield tert-butyl 3′-hydroxy-8′-methoxyspiro [azetidine-3,6′-benzo[c]chromene]-1-carboxylate (120 mg, 0.330 mmol, 81%) as a light yellow solid. R_{f}=0.3 (EtOAc/hexane 20%) as yellowish solid. ¹H NMR (400 MHz, DMSO) δ 9.70 (s, 1H), 7.68 (d, J=8.6 Hz, 1H), 7.61 (d, J=8.5 Hz, 1H), 7.05 (d, J=2.6 Hz, 1H), 7.00 (dd, J=8.6, 2.6 Hz, 1H), 6.51 (dd, J=8.5, 2.4 Hz, 1H), 6.45 (d, J=2.4 Hz, 1H), 4.19 (d, J=9.6 Hz, 2H), 4.10 (d, J=9.7 Hz, 2H), 3.83 (s, 3H), 1.41 (s, 9H).

### Step 4: Synthesis of spiro[azetidine-3,6′-benzo[c]chromene]-3′,8′-diol hydrobromide

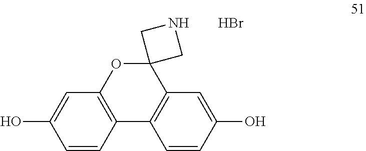 [SMILES: Br ; Oc1ccc2c(c1)OC1(CNC1)c1cc(O)ccc1-2]

BBr₃ (0.54 ml, 0.54 mmol, 2.0 eq) was added to a solution of tert-butyl 3′-hydroxy-8′-methoxyspiro[azetidine-3,6′-benzo[c]chromene]-1-carboxylate (100 mg, 0.270 mmol, 1.0 eq.) in DCM (5 mL) at 0° C. and the mixture was allowed to warm to room temperature overnight. Methanol was added to the mixture at 0° C. was concentrated under vacuum and loaded on silica then purified by FC eluent MeOH/DCM 0% to 8% to give spiro[azetidine-3,6′-benzo[c]chromene]-3′,8′-diol hydrobromide (40 mg, 44%) as a white solid. MS (ESI+): m/z=256. ¹H NMR (400 MHz, DMSO) δ 9.76 (d, J=22.3 Hz, 2H), 9.42 (s, 1H), 8.91 (s, 1H), 7.59 (t, J=8.7 Hz, 2H), 7.06 (d, J=2.4 Hz, 1H), 6.95-6.83 (m, 1H), 6.54 (dd, J=8.4, 2.4 Hz, 1H), 6.49 (d, J=2.3 Hz, 1H), 4.38 (dt, J=12.6, 6.8 Hz, 2H), 4.24 (ddd, J=12.2, 7.4, 4.0 Hz, 2H).

### I) Ester “A” ring analogues with peptide substitution

### Synthesis of 3-acetoxy-6-oxo-6H-benzo[c]chromene-8-carboxylic acid as the common intermediate

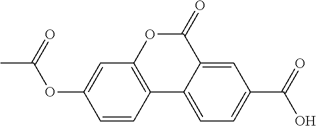 [SMILES: CC(=O)Oc1ccc2c(c1)oc(=O)c1cc(C(=O)O)ccc12]

Acetylchlorid (0.36 ml, 5.2 mmol) was added at 0° C. to a suspension of 3-hydroxy-6-oxo-6H-benzo[c]chromene-8-carboxylic acid (2 (600 mg, 2.34 mmol) in THF (8 mL) and the reaction mixture was allowed to warm to room temperature overnight. The reaction mixture is still a suspension (nothing solubilises). HCl 1M was added to the suspension and stirred 30 minutes at room temperature. The white suspension was filtered off and the solid was washed with cooled water and dried under vacuum to give (17) as a white solid (400 mg, 57%). ¹H NMR (400 MHz, CDCl₃) δ 8.72 (d, J=1.8 Hz, 1H), 8.53 (d, J=8.5 Hz, 1H), 8.47 (d, J=8.8 Hz, 1H), 8.39 (dd, J=8.4, 1.9 Hz, 1H), 7.34 (d, J=2.2 Hz, 1H), 7.26 (dd, J=8.7, 2.3 Hz, 1H), 2.33 (s, 3H).

### General procedure peptide coupling using FDPP and deprotection using potassium carbonate

### Synthesis of 8-((2-morpholinoethyl)carbamoyl)-6-oxo-6H-benzo[c]chromen-3-yl acetate (52)

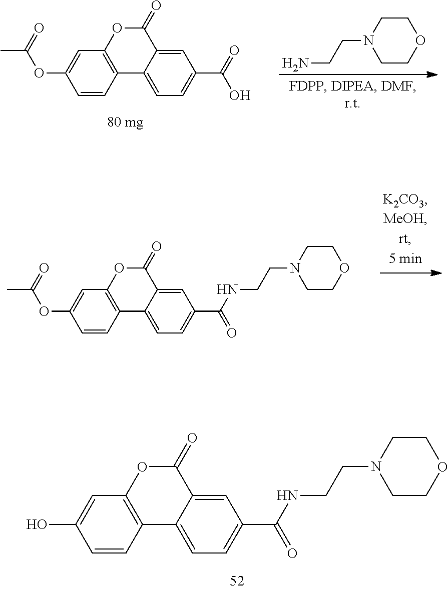 [SMILES: CC(=O)Oc1ccc2c(c1)oc(=O)c1cc(C(=O)O)ccc12 ; NCCN1CCOCC1 ; CC(=O)Oc1ccc2c(c1)oc(=O)c1cc(C(=O)NCCN3CCOCC3)ccc12 ; O=C(NCCN1CCOCC1)c1ccc2c(c1)c(=O)oc1cc(O)ccc12]

### Step 1: Synthesis of 8-((2-morpholinoethyl)carbamoyl)-6-oxo-6H-benzo[c]chromen-3-yl acetate

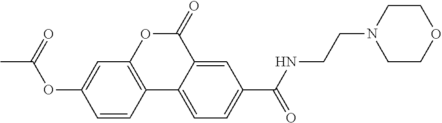 [SMILES: CC(=O)Oc1ccc2c(c1)oc(=O)c1cc(C(=O)NCCN3CCOCC3)ccc12]

DIPEA (0.15 ml, 0.86 mmol) was added to a solution of 3-acetoxy-6-oxo-6H-benzo[c]chromene-8-carboxylic acid (80 mg, 0.21 mmol) in DMF (2 mL) followed by Pentafluorophenyldiphenylphosphinate (91 mg, 0.24 mmol) and the mixture was stirred 15 min then add 2-morpholinoethan-1-amine (28 mg, 0.21 mmol) dropwise and stirring continued for 1 h. The reaction mixture was extracted with EtOAc and bicarbonate 1/2 saturated solution 3 times. The combined organic phases were dried over sodium sulfate and concentrated under vacuum. The crude was purified by MPLC (SiO₂, MeOH/DCM from 0% to 10%) to afford 8-((2-morpholinoethyl)carbamoyl)-6-oxo-6H-benzo[c]chromen-3-yl acetate (45 mg, 51%) R_{f}=0.3 (10% MeOH/DCM). ¹H NMR (400 MHz, CDCl₃) δ 8.69 (d, J=1.9 Hz, 1H), 8.39 (dd, J=8.4, 2.0 Hz, 1H), 8.17 (d, J=8.4 Hz, 1H), 8.10 (d, J=8.7 Hz, 1H), 7.20 (d, J=2.2 Hz, 1H), 7.17 (dd, J=8.6, 2.3 Hz, 1H), 7.00 (s, 1H), 3.78 (t, J=4.6 Hz, 4H), 3.63 (q, J=5.6 Hz, 2H), 2.68 (d, J=4.6 Hz, 2H), 2.57 (s, 4H), 2.36 (s, 3H).

### Step 2: Synthesis of 3-hydroxy-N-(2-morpholinoethyl)-6-oxo-6H-benzo[c]chromene-8-carboxamide

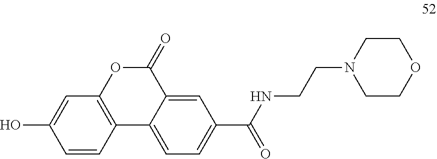 [SMILES: O=C(NCCN1CCOCC1)c1ccc2c(c1)c(=O)oc1cc(O)ccc12]

Potassium carbonate (36 mg, 0.26 mmol) was added at rt to solution of 8-((2-morpholinoethyl)carbamoyl)-6-oxo-6H-benzo[c]chromen-3-yl acetate (36 mg, 0.088 mmol) in MeOH and the reaction mixture was stirred at room temperature 10 min. The mixture was loaded on silica gel and purified by MPLC (SiO₂, MeOH/dichloromethane 0% to 10%) to afford 3-hydroxy-N-(2-morpholinoethyl)-6-oxo-6H-benzo[c]chromene-8-carboxamide UA0350 (23 mg, 71%). R_{f}=0.2 (10% MeOH/DCM). ¹H NMR (400 MHz, DMSO) δ 10.48 (s, 1H), 8.76 (t, J=5.6 Hz, 1H), 8.68 (d, J=1.9 Hz, 1H), 8.36 (d, J=8.6 Hz, 1H), 8.29 (dd, J=8.5, 1.9 Hz, 1H), 8.24-8.19 (m, 1H), 6.90-6.84 (m, 1H), 6.78 (d, J=2.4 Hz, 1H), 3.58 (t, J=4.6 Hz, 4H), 3.43 (q, J=6.5 Hz, 2H), 2.43 (s, 4H) (2 missing protons are overshadowed by solvent).

### Synthesis of 3-hydroxy-6-oxo-N-(2-(piperidin-1-yl)ethyl)-6H-benzo[c]chromene-8-carboxamide (53)

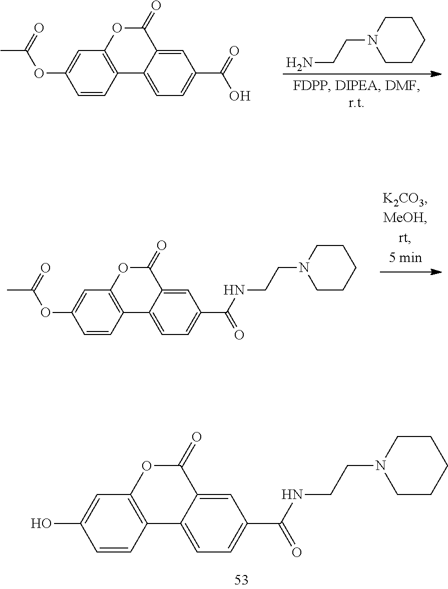 [SMILES: CC(=O)Oc1ccc2c(c1)oc(=O)c1cc(C(=O)O)ccc12 ; NCCN1CCCCC1 ; CC(=O)Oc1ccc2c(c1)oc(=O)c1cc(C(=O)NCCN3CCCCC3)ccc12 ; O=C(NCCN1CCCCC1)c1ccc2c(c1)c(=O)oc1cc(O)ccc12]

### Step 1: Synthesis of 6-oxo-8-((2-(piperidin-1-yl)ethyl)carbamoyl)-6H-benzo[c]chromen-3-yl acetate

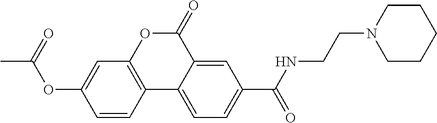 [SMILES: CC(=O)Oc1ccc2c(c1)oc(=O)c1cc(C(=O)NCCN3CCCCC3)ccc12]

Compound was prepared according to general procedure starting from 3-acetoxy-6-oxo-6H-benzo[c]chromene-8-carboxylic acid (120 mg, 0.320 mmol), Pentafluorophenyl-diphenylphosphinate (136 mg, 0.35 mmol), 2-(piperidin-1-yl)ethan-1-amine (41 mg, 0.32 mmol) and DIPEA (0.224 ml, 1.29 mmol) to afford after purification by MPLC (SiO₂, MeOH/DCM 0% to 10%) 6-oxo-8-((2-(piperidwiin-1-yl)ethyl)carbamoyl)-6H-benzo[c]chromen-3-yl acetate 19 (65 mg, 49%) as a white solid. R_{f}=0.3 (MeOH/DCM 10%). ¹H NMR (400 MHz, CDCl₃) δ 8.74 (d, J=1.9 Hz, 1H), 8.40 (dd, J=8.4, 1.9 Hz, 1H), 8.16 (d, J=8.5 Hz, 1H), 8.09 (d, J=8.7 Hz, 1H), 7.54-7.36 (m, 1H), 7.22-7.13 (m, 2H), 3.63 (q, J=5.5 Hz, 2H), 2.68 (t, J=5.8 Hz, 2H), 2.56 (s, 4H), 2.36 (s, 3H), 1.75-1.60 (m, 4H), 1.51 (s, 2H).

### Step 2: Synthesis of 3-hydroxy-6-oxo-N-(2-(piperidin-1-yl)ethyl)-6H-benzo[c]chromene-8-carboxamide

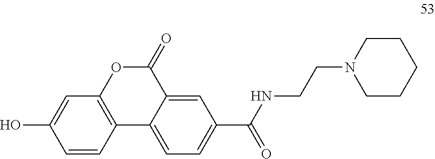 [SMILES: O=C(NCCN1CCCCC1)c1ccc2c(c1)c(=O)oc1cc(O)ccc12]

3-hydroxy-6-oxo-N-(2-(piperidin-1-yl)ethyl)-6H-benzo[c]chromene-8-carboxamide was prepared according to GP5 starting from 6-oxo-8-((2-(piperidin-1-yl)ethyl)carbamoyl)-6H-benzo[c]chromen-3-yl acetate 19 (49 mg, 0.12 mmol) and potassium carbonate (50 mg, 0.36 mmol) to afford after purification by MPLC (SiO₂, MeOH/DCM 5% to 35%) 3-hydroxy-6-oxo-N-(2-(piperidin-1-yl)ethyl)-6H-benzo[c]chromene-8-carboxamide 53 (15 mg, 34%) as a white solid. R_{f}=0.3 (MeOH/DCM 20%). ¹H NMR (400 MHz, DMSO) δ 10.52 (s, 1H), 8.83 (s, 1H), 8.68 (d, J=1.9 Hz, 1H), 8.36 (d, J=8.6 Hz, 1H), 8.29 (dd, J=8.5, 1.9 Hz, 1H), 8.22 (d, J=8.8 Hz, 1H), 6.87 (dd, J=8.7, 2.4 Hz, 1H), 6.78 (d, J=2.4 Hz, 1H), 3.47 (d, J=21.5 Hz, 5H), 1.65-1.19 (m, 9H).

### Synthesis of-hydroxy-N-(2-(4-methylpiperazin-1-yl)ethyl)-6-oxo-6H-benzo[c]chromene-8-carboxamide (54)

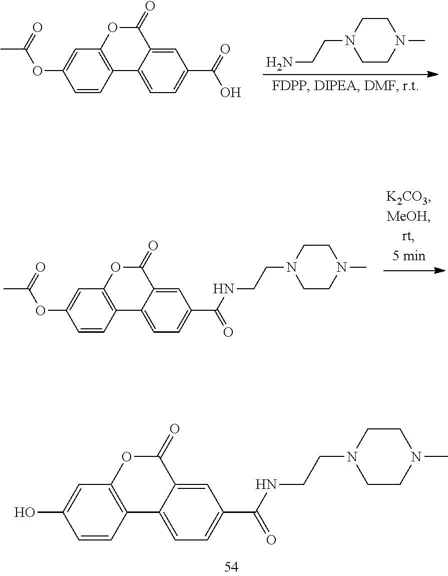

### Step 1: Synthesis of 8-((2-(4-methylpiperazin-1-yl)ethyl)carbamoyl)-6-oxo-6H-benzo[c]chromen-3-yl acetate

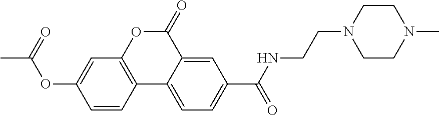 [SMILES: CC(=O)Oc1ccc2c(c1)oc(=O)c1cc(C(=O)NCCN3CCN(C)CC3)ccc12]

Compound was prepared according to GP4 starting from 3-acetoxy-6-oxo-6H-benzo[c]chromene-8-carboxylic acid (120 mg, 0.260 mmol) Pentafluorophenyldiphenylphosphinate (113 mg, 0.290 mmol) and DIPEA (0.187 ml, 1.070 mmol) to afford after purification by MPLC (SiO₂, MeOH/DCM 0% to 10%) 8-((2-(4-methylpiperazin-1-yl)ethyl)carbamoyl)-6-oxo-6H-benzo[c]chromen-3-yl acetate 20 (73 mg, 59%) as a white solid. R_{f}=0.3 eluent (MeOH/DCM 10%). ¹NMR (400 MHz, CDCl₃) δ 8.68 (d, J=1.9 Hz, 1H), 8.39 (dd, J=8.4, 2.0 Hz, 1H), 8.16 (d, J=8.5 Hz, 1H), 8.09 (d, J=8.7 Hz, 1H), 7.19 (d, J=2.2 Hz, 1H), 7.16 (dd, J=8.6, 2.3 Hz, 1H), 7.11 (s, 1H), 3.62 (q, J=5.6 Hz, 2H), 2.73-2.58 (m, 10H), 2.37 (d, J=5.2 Hz, 6H).

### Step 2: Synthesis of-hydroxy-N-(2-(4-methylpiperazin-1-yl)ethyl)-6-oxo-6H-benzo[c]chromene-8-carboxamide

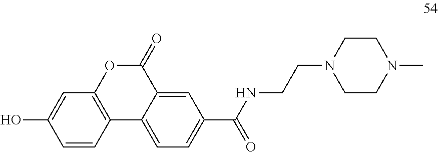 [SMILES: CN1CCN(CCNC(=O)c2ccc3c(c2)c(=O)oc2cc(O)ccc23)CC1]

3-hydroxy-N-(2-(4-methylpiperazin-1-yl)ethyl)-6-oxo-6H-benzo[c]chromene-8-carboxamide was prepared according to GP5 starting from 8-((2-(4-methylpiperazin-1-yl)ethyl)carbamoyl)-6-oxo-6H-benzo[c]chromen-3-yl acetate 20 (60 mg, 0.14 mmol) and potassium carbonate (39 mg, 0.28 mmol) to afford after purification by MPLC (RP-C₁₈, MeOH/water 0% to 95%) 3-hydroxy-N-(2-(4-methylpiperazin-1-yl)ethyl)-6-oxo-6H-benzo[c]chromene-8-carboxamide (27 mg, 51%). R_{f}=0.1 eluent (MeOH/DCM 30%). ¹H NMR (400 MHz, DMSO) δ 8.75 (t, J=5.6 Hz, 1H), 8.68 (d, J=1.8 Hz, 1H), 8.36 (d, J=8.6 Hz, 1H), 8.29 (dd, J=8.5, 1.9 Hz, 1H), 8.22 (d, J=8.9 Hz, 1H), 8.18 (s, 1H), 6.88 (dd, J=8.7, 2.4 Hz, 1H), 6.78 (d, J=2.4 Hz, 1H), 3.47-3.40 (m, 2H), 2.48-2.30 (m, 10H), 2.20 (s, 3H).

### J) Ester “A” group analogs with inverse amide substitution

The syntheses of inverse amides was based on a common intermediate that is described below.

### Synthesis of N-(3-(benzyloxy)-6-oxo-6H-benzo[c]chromen-8-yl)-2-chloroacetamide

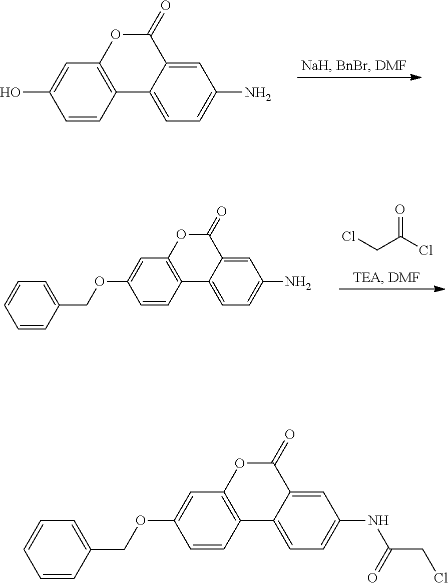 [SMILES: Nc1ccc2c(c1)c(=O)oc1cc(O)ccc12 ; Nc1ccc2c(c1)c(=O)oc1cc(OCc3ccccc3)ccc12 ; O=C(Cl)CCl ; O=C(CCl)Nc1ccc2c(c1)c(=O)oc1cc(OCc3ccccc3)ccc12]

### Step 1: Synthesis of 8-amino-3-(benzyloxy)-6H-benzo[c]chromen-6-one

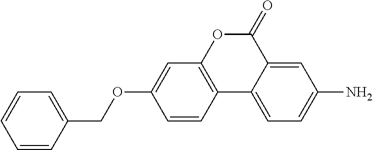 [SMILES: Nc1ccc2c(c1)c(=O)oc1cc(OCc3ccccc3)ccc12]

8-amino-3-hydroxy-6H-benzo[c]chromen-6-one 15 (864 mg, 3.80 mmol) was dissolved in DMF (13 mL) then cooled to 0° C. Subsequently NaH (152 mg, 3.80 mmol) was added in one portion. Upon stirring for 15 min benzyl chloride (0.44 ml, 3.80 mmol) was added dropwise and the reaction mixture was allowed to warm to r.t. and stirring overnight was continued. Following the reaction was quenched with half-saturated NaHCO₃ solution and extracted with ethyl acetate (3×25 ml). The combined organic layers were dried over Na₂SO₄ and concentrated under reduced pressure. The crude product was purified by MPLC (SiO₂, ethyl acetate/Hex 0-50%) to afford 8-amino-3-(benzyloxy)-6H-benzo[c]chromen-6-one (738 mg, 61%) as an ocher colored solid. ¹H NMR (400 MHz, DMSO) δ 8.02 (dd, J=17.2, 8.7 Hz, 2H), 7.51-7.34 (m, 6H), 7.14 (dd, J=8.7, 2.6 Hz, 1H), 7.06-6.95 (m, 2H), 5.79 (s, 2H), 5.19 (s, 2H).

### Step 2: Synthesis of N-(3-(benzyloxy)-6-oxo-6H-benzo[c]chromen-8-yl)-2-chloroacetamide

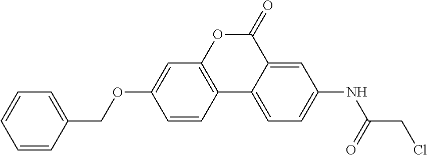 [SMILES: O=C(CCl)Nc1ccc2c(c1)c(=O)oc1cc(OCc3ccccc3)ccc12]

8-amino-3-(benzyloxy)-6H-benzo[c]chromen-6-one (738 mg, 2.33 mmol) was added to a solution of DMF (16 ml) containing TEA (0.324 ml, 2.56 mmol). The mixture was stirred for 10 min at room temperature. Chloroacetylchloride (0.205 ml, 2.33 mmol) was added to the above mixture, maintaining the temperature between 0 and 5° C. The obtained solution was then stirred at room temperature for 4-6 h. The completion of reaction was monitored with TLC. The solution was then added onto crushed ice and the separated precipitates were filtered and dried under vacuum. The product was recrystallized from methanol to afford N-(3-(benzyloxy)-6-oxo-6H-benzo[c]chromen-8-yl)-2-chloroacetamide (833 mg, 91%) as a lightly yellowish solid. ¹H NMR (400 MHz, DMSO) δ 10.72 (s, 1H), 8.52 (d, J=2.4 Hz, 1H), 8.31 (d, J=8.8 Hz, 1H), 8.19 (d, J=8.8 Hz, 1H), 8.03 (dd, J=8.8, 2.4 Hz, 1H), 7.48-7.35 (m, 5H), 7.10-7.06 (m, 2H), 5.22 (s, 2H), 4.32 (s, 2H).

### Synthesis of N-(3-(hydroxy)-6-oxo-6H-benzo[c]chromen-8-yl)-2-morpholinoacetamide (55)

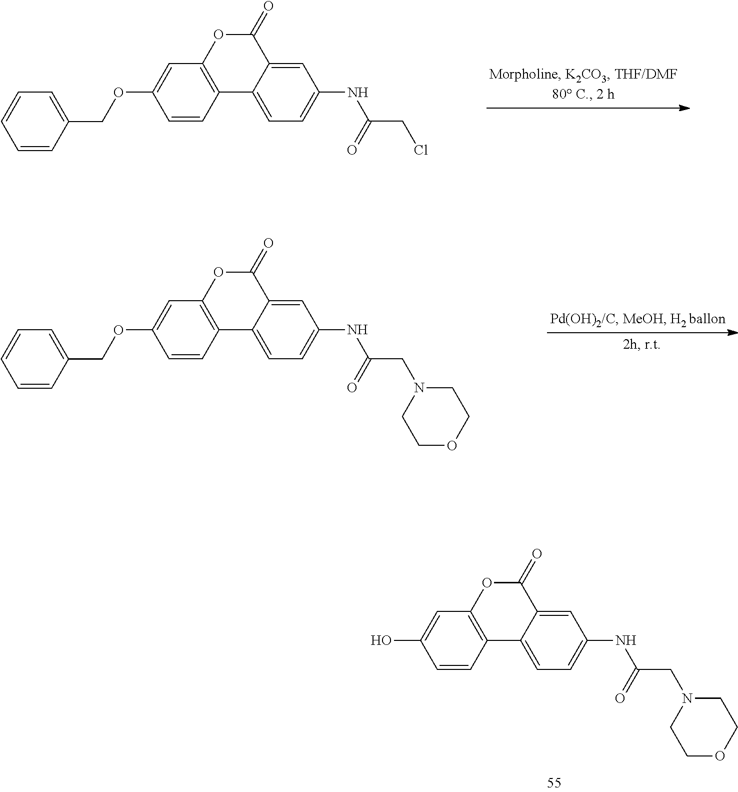 [SMILES: O=C(CCl)Nc1ccc2c(c1)c(=O)oc1cc(OCc3ccccc3)ccc12 ; O=C(CN1CCOCC1)Nc1ccc2c(c1)c(=O)oc1cc(OCc3ccccc3)ccc12 ; O=C(CN1CCOCC1)Nc1ccc2c(c1)c(=O)oc1cc(O)ccc12]

### Step 1: Synthesis of N-(3-(benzyloxy)-6-oxo-6H-benzo[c]chromen-8-yl)-2-morpholinoacetamide

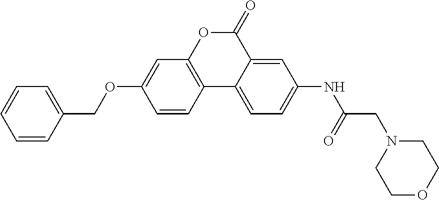 [SMILES: O=C(CN1CCOCC1)Nc1ccc2c(c1)c(=O)oc1cc(OCc3ccccc3)ccc12]

N-(3-(benzyloxy)-6-oxo-6H-benzo[c]chromen-8-yl)-2-chloroacetamide (60 mg, 0.15 mmol) was suspended in THF (5 ml) and the potassium carbonate (42 mg, 0.30 mmol) was added in one portion. The minimum amount of DMF (2-3 ml) were added dropwise in order to solubilize the suspension. Then morpholine (0.014 mL, 0.17 mmol) was added via syringe and the reaction was heated to 80° C. for 2 h. Upon complete consumption of the starting material (as indicated by TLC) the reaction was allowed to cool down to r.t. and then the mixture was concentrated under reduced pressure. The crude product was purified by MPLC (SiO₂, MeOH in DCM 0-10%) to afford N-(3-(benzyloxy)-6-oxo-6H-benzo[c]chromen-8-yl)-2-morpholinoacetamide (46 mg, 0.10 mmol, 68%) as a white solid. ¹H NMR (400 MHz, DMSO) δ 10.17 (s, 1H), 8.60 (d, J=2.3 Hz, 1H), 8.29 (d, J=8.9 Hz, 1H), 8.21 (d, J=8.7 Hz, 1H), 8.13 (dd, J=8.8, 2.4 Hz, 1H), 7.50-7.35 (m, 5H), 7.11-7.06 (m, 2H), 5.23 (s, 2H), 3.66 (t, J=4.7 Hz, 4H), 3.19 (s, 2H), 2.55-2.52 (m, 4H).

### Step 2: Synthesis of N-(3-hydroxy-6-oxo-6H-benzo[c]chromen-8-yl)-2-morpholinoacetamide

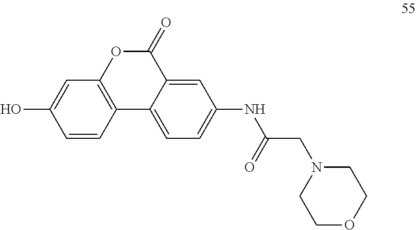 [SMILES: O=C(CN1CCOCC1)Nc1ccc2c(c1)c(=O)oc1cc(O)ccc12]

A solution of N-(3-(benzyloxy)-6-oxo-6H-benzo[c]chromen-8-yl)₂-morpholinoacetamide (40 ng, 0.090 mmol) and Pd(OH)₂/C (7 mg, 0.009 mmol) in MeOH (2n) and DCM (2 ml) was stirred under hydrogen at atmospheric pressure overnight. The reaction mixture was filtered over a pad of celite and the solvent was evaporated under vacuum to give N-(3-hydroxy-6-oxo-6H-benzo[c]chromen-8-yl)-2-morpholinoacetamide (25 mg, 78%) as an off-white solid. H NMR (400 MHz, DMSO) δ 10.26 (s, 11-1) 10.14 (s, 1H), 8.57 (d, J=2.3 Hz, 1H), 8.23 (d, J=9.0 Hz, 11H), 8.10 (d, J=8.8 Hz. 2H). 6.83 (dd, J=8.7, 2.5 Hz, 1H), 6.75 (d, J=2.4 Hz, 1H), 3.65 (t, J=4.8 Hz, 4H), 3.18 (s, 2H) (clean, but 4 aliphatic protons are overshadowed by solvent)

### Synthesis of N-(3-(hydroxy)-6-oxo-6H-benzo[c]chromen-8-yl)-2-(piperidin-1-yl)acetamide (56)

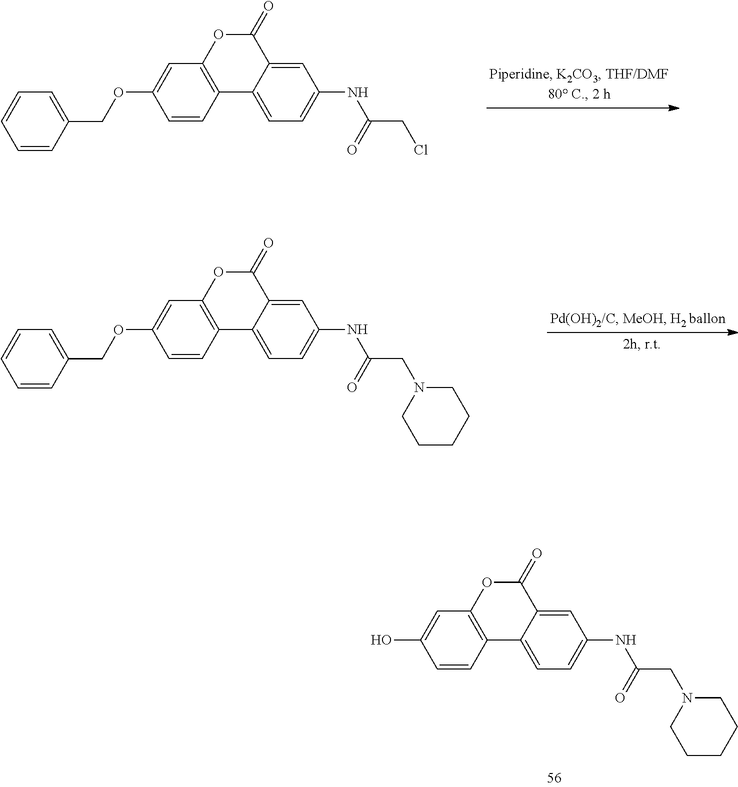 [SMILES: O=C(CCl)Nc1ccc2c(c1)c(=O)oc1cc(OCc3ccccc3)ccc12 ; O=C(CN1CCCCC1)Nc1ccc2c(c1)c(=O)oc1cc(OCc3ccccc3)ccc12 ; O=C(CN1CCCCC1)Nc1ccc2c(c1)c(=O)oc1cc(O)ccc12]

### Step 1: Synthesis of N-(3-benzyloxy)-6-oxo-6H-benzo[c]chromen-8-yl)-2-(piperidin-1-yl)acetamide

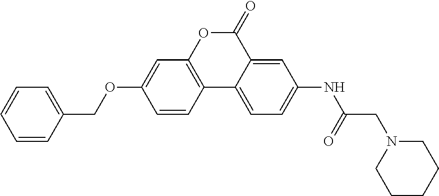 [SMILES: O=C(CN1CCCCC1)Nc1ccc2c(c1)c(=O)oc1cc(OCc3ccccc3)ccc12]

N-(3-(benzyloxy)-6-oxo-6H-benzo[c]chromen-8-yl)-2-chloroacetamide (200 mg, 0.510 mmol) was suspended in THF (5 ml) and the potassium carbonate (140 mg, 1.02 mmol) was added in one portion. The minimum amount of DMF (5-6 ml) were added in order to solubilize the suspension. Then piperidine (0.055 mL, 0.56 mmol) was added dropwise via syringe and the reaction was heated to 80° C. for 2 h. Upon complete consumption of the starting material (as indicated by TLC) the reaction was allowed to cool down to r.t. and then the mixture was concentrated under reduced pressure. The crude product was purified by flash column chromatography (MeOH in DCM 0-10%) to obtain N-(3-(benzyloxy)-6-oxo-6H-benzo[c]chromen-8-yl)-2-(piperidin-1-yl acetamide (154 mg, 0.51 mmol, 69%) as a white solid. ¹H NMR (400 MHz, DMSO) δ 10.10 (s, 1H), 8.61 (d, J=2.3 Hz, 1H), 8.28 (d, J=8.9 Hz, 1H), 8.20 (d, J=8.8 Hz, 1H), 8.12 (dd, J=8.8, 2.4 Hz, 1H), 7.53-7.37 (m, 5H), 7.13-7.05 (m, 2H), 5.23 (s, 2H), 3.13 (s, 2H), 2.47 (d, J=5.0 Hz, 4H), 1.58 (p, J=5.6 Hz, 4H), 1.41 (q, J=6.0 Hz, 2H).

### Step 2: Synthesis N-(3-hydroxy-6-oxo-6H-benzo[c]chromen-8-yl)-2-(piperidin-1-yl)acetamide

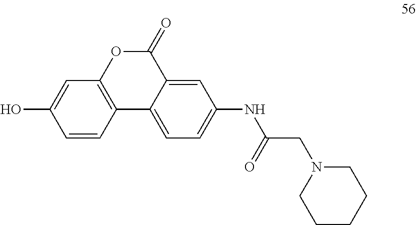 [SMILES: O=C(CN1CCCCC1)Nc1ccc2c(c1)c(=O)oc1cc(O)ccc12]

A solution of N-(3-(benzyloxy)-6-oxo-6H-benzo[c]chromen-8-yl)-2-(piperidin-1-yl)acetamide (154 mg, 0.350 mmol) and Pd(OH)₂/C (34 mg, 0.035 mmol) in MeOH (3 ml) and DCM (3 ml) was stirred under hydrogen at atmospheric pressure overnight. The reaction mixture was filtered over a pad of celite and the solvent was evaporated under vacuum to give N-(3-hydroxy-6-oxo-6H-benzo[c]chromen-8-yl)-2-(piperidin-1-yl)acetamide (95 mg, 77%) as a dark yellow solid. ¹H NMR (400 MHz, DMSO) δ 10.07 (s, 1H), 8.58 (d, J=2.3 Hz, 1H), 8.21 (d, J=8.9 Hz, 1H), 8.09 (dd, J=8.8, 2.8 Hz, 2H), 6.83 (dd, J=8.7, 2.4 Hz, 1H), 6.74 (d, J=2.4 Hz, 1H), 3.12 (s, 2H), 2.47 (d, J=5.6 Hz, 4H), 1.59 (q, J=5.6 Hz, 4H), 1.41 (q, J=6.2 Hz, 2H).

MS (ESI+): m/z=353

### Synthesis of N-(3-hydroxy)-6-oxo-6H-benzo[c]chromen-8-yl)-2-(4-methylpiperazin-1-yl)acetamide (57)

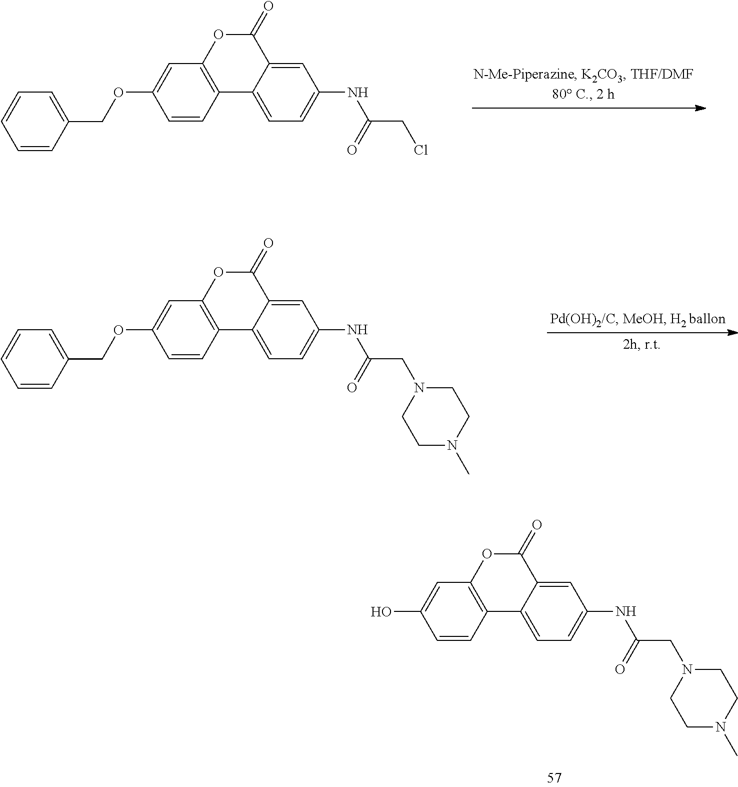 [SMILES: O=C(CCl)Nc1ccc2c(c1)c(=O)oc1cc(OCc3ccccc3)ccc12 ; CN1CCN(CC(=O)Nc2ccc3c(c2)c(=O)oc2cc(OCc4ccccc4)ccc23)CC1 ; CN1CCN(CC(=O)Nc2ccc3c(c2)c(=O)oc2cc(O)ccc23)CC1]

### Step 1: Synthesis of N-(3-(benzyloxyl)-6-oxo-61-H-benzo[c]chromen-8-yl)-2-(piperidin-1-yl)acetamide

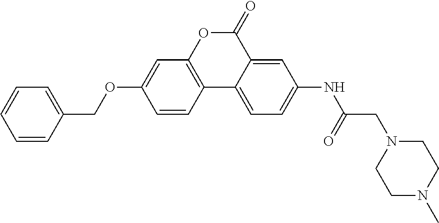 [SMILES: CN1CCN(CC(=O)Nc2ccc3c(c2)c(=O)oc2cc(OCc4ccccc4)ccc23)CC1]

N-(3-(benzyloxy)-6-oxo-6H-benzo[c]chromen-8-yl)-2-chloroacetamide (200 mg, 0.510 mmol) was suspended in THF (5 ml) and the potassium carbonate (140 mg, 1.02 mmol) was added in one portion. The minimum amount of DMF (5-6 ml) were added in order to solubilize the suspension. Then 1-methylpiperazine (0.062 mL, 0.56 mmol) was added dropwise via syringe and the reaction was heated to 80° C. for 2 h. Upon complete consumption of the starting material (as indicated by TLC) the reaction was allowed to cool down to r.t. and then the mixture was concentrated under reduced pressure. The crude product was purified by flash column chromatography (MeOH in DCM 0-20%) to obtain N-(3-(benzyloxy)-6-oxo-6H-benzo[c]chromen-8-yl)-2-(piperidin-1-yl)acetamide (148 mg, 64%) as a white solid. ¹H NMR (400 MHz, DMSO) δ 10.21 (s, 1H), 8.59 (d, J=2.3 Hz, 1H), 8.29 (d, J=8.9 Hz, 1H), 8.20 (d, J=8.8 Hz, 1H), 8.11 (dd, J=8.8, 2.3 Hz, 1H), 7.55-7.30 (m, 5H), 7.14-7.03 (m, 2H), 5.23 (s, 2H), 3.17 (s, 2H), 2.68-2.66 (m, 8H), 2.39 (s, 3H).

### Step 2: Synthesis of N-(3-hydroxy-6-oxo-6H-benzo[c]chromen-8-yl)-2-(4-methylpiperazin-1-yl)acetamide

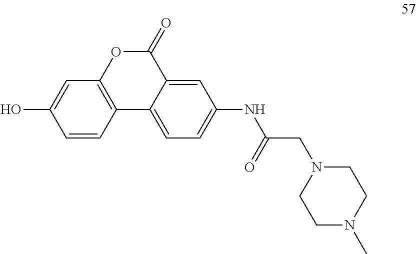 [SMILES: CN1CCN(CC(=O)Nc2ccc3c(c2)c(=O)oc2cc(O)ccc23)CC1]

A solution of N-(3-(benzyloxy)-6-oxo-6H-benzo[c]chromen-8-yl)-2-(piperidin-1-yl)acetamide (148 mg, 0.320 mmol) Pd(OH)₂/C (40 mg, 0.032 mmol) in MeOH (3 ml) and DCM (3 ml was stirred under hydrogen at atmospheric pressure overnight. The reaction mixture was filtered over a pad of celite and the solvent was evaporated under vacuum to give N-(3-hydroxy-6-oxo-6H-benzo[c]chromen-8-yl)-2-(4-methy piperazin-1-yl)acetamide (69 mg, 58%) as a pale yellow solid. MS (ESI+): m/z=368.

### K) Thionoester “A” group analogues

### Synthesis of 3,8-dimethoxy-6H-benzo[c]chromene-6-thione (58)

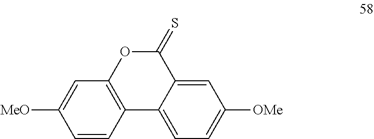 [SMILES: COc1ccc2c(c1)oc(=S)c1cc(OC)ccc12]

A mixture of 3,8-dimethoxy-6H-benzo[c]chromen-6-one (previously described above) (140 mg, 0.154 mmol) and lawesson's reagent (552 mg, 1.34 mmol) were refluxed in toluene o.n. The reaction was monitored by TLC which showed that reaction is not complete so lawesson's reagent (884 mg, 2.19 mmol) were added and reflux continued overnight. The reaction mixture was filtered off and the solvent was evaporated under vacuum. The crude was purified by MPLC (SiO₂, EtOAc/cyclohexane 0% to 25%) to afford 3,8-dimethoxy-6H-benzo[c]chromene-6-thione (110 mg, 74%) as a yellow solid. R_{f}=0.4 (EtOAc/hexane 20%) yellow solid. ¹H NMR (400 MHz, CDCl3) δ 8.21 (d, J=2.8 Hz, 1H) 7.92 (dd, J=8.9, 6.7 Hz, 2H), 7.39 (dd J=8.9, 2.8 Hz, 1H), 7.03 (d, J=2.6 Hz. 1H), 6.99-6.95 (dd, 1H), 3.96 (s, 3H), 3.88 (s, 3H).

### Synthesis of 3,8-dihydroxy-6H-benzo c thiochromen-6-one (9)

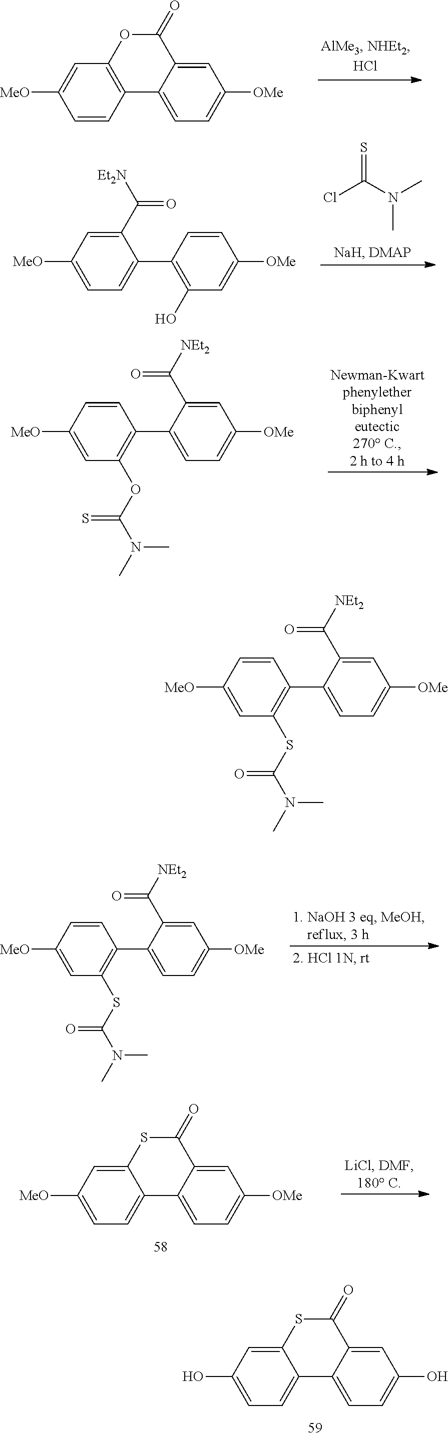

58 was prepared in 4 steps from 17 according to a described procedure in Org. Lett., Vol. 7, No. 3, 2005, 411-414. The product was obtained as a white solid. The analytical data fully matched to the one that was previously reported in the literature.

### Step 5: Synthesis of 3,8-dihydroxy-6H-benzo[c]thiochromen-6-one

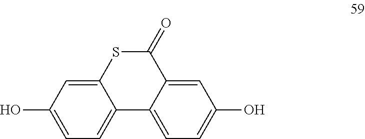 [SMILES: O=c1sc2cc(O)ccc2c2ccc(O)cc12]

In a sealed tube, a mixture of Lithium Chloride (65 mg, 1.5 mmol) and 3,8-dimethoxy-6H-benzo[c]thiochromen-6-one (70 mg, 0.26 mmol) in DMF (1 mL) was heated at 130° C. for 2 days. The solvent was evaporated under vacuum and the crude was loaded on silica gel and was purified by MPLC (SiO₂, Methanol/Dichloromethane 0% to 10%) to afford 3,8-dihydroxy-6H-benzo[c]thiochromen-6-one (28 mg, 45%). as a yellow solid. ¹H NMR (400 MHz, DMSO) δ 10.19 (s, 2H), 8.32 (dd, J=15.6, 9.1 Hz, 2H), 7.53 (d, J=2.8 Hz, 1H), 7.31 (dd, J=8.9, 2.9 Hz, 1H), 6.92 (dd, J=8.9, 2.6 Hz, 1H), 6.87 (d, J=2.5 Hz, 1H).

### Synthesis of 3,8-dihydroxy-6H-benzo[c]thiochromene 5,5-dioxide (62)

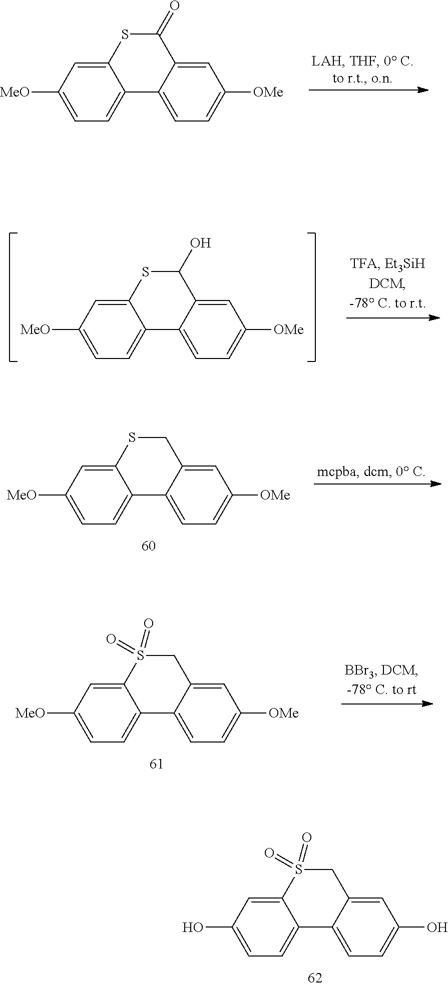

### Step 1: Synthesis of 3,8-dimethoxy-6H-benzo[c]thiochromene (60)

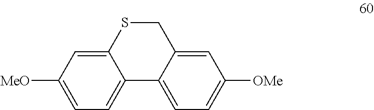 [SMILES: COc1ccc2c(c1)CSc1cc(OC)ccc1-2]

LAH (35 mg, 0.91 mmol) was added to solution of 3,8-dimethoxy-6H-benzo[c]thiochromen-6-one (250 mg, 0.910 mmol) in DCM (10 ml) at 0° C. and the mixture was stirred overnight at room temperature. workup: add 10 ml Et₂O followed by 0.05 ml of MeOH, NaOH 1N 0.025 ml then water 3 drops and stirring continued for 15 min. Na₂SO₄ was added and the reaction mixture was filtered off and concentrated under vacuum. The crude was dissolved in DCM (5 ml) and cooled down to −78° C., TFA (0.354 mL, 4.59 mmol) was added dropwise and stirred 60 min at −78° C., then EtSi₃H (0.290 ml, 1.84 mmol) was added and the reaction was allowed to warm to room temperature overnight. The reaction mixture was washed with Na₂CO₃ saturated solution and the organic layer was dried over sodium sulfate and concentrated under vacuum to afford 230 mg of crude material, which was triturated in Et₂O to afford 3,8-dimethoxy-6H-benzo[c]thiochromene (160 mg, 67%) as a white solid. ¹H NMR (400 MHz, CDCl₃) δ 7.64 (d, J=8.7 Hz, 1H), 7.54 (d, J=8.6 Hz, 1H), 6.94 (d, J=2.7 Hz, 1H), 6.89 (dd, J=8.6, 2.7 Hz, 1H), 6.81 (dd, J=8.7, 2.7 Hz, 1H), 6.77 (d, J=2.7 Hz, 1H), 3.84 (s, 3H), 3.82 (s, 3H), 3.81 (s, 2H).

### Step 2: Synthesis of 3,8-dimethoxy-6H-benzo[c]thiochromene 5,5-dioxide (61)

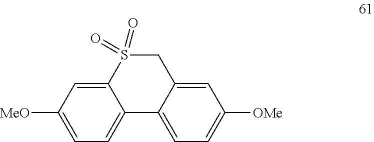 [SMILES: COc1ccc2c(c1)CS(=O)(=O)c1cc(OC)ccc1-2]

m-CPBA (150 mg, 0.62 mmol) was added to a solution of 3,8-dimethoxy-6H-benzo[c]thiochromene (80 mg, 0.31 mmol) in dichloromethane (4 ml) at 0° C. and the mixture was allowed to warm to room temperature over 2 h. 1M Na₂S₂O₃ solution was added to the reaction mixture. The aqueous phase was extracted with EtOAc and the organic phase was washed with bicarbonate saturated solution twice. The organic phase was dried over sodium sulfate. The organic phase was concentrated under vacuum and filtered over a pad of celite using EtOAc then concentrated to afford 3,8-dimethoxy-6H-benzo[c]thiochromene 5,5-dioxide (66 mg, 73%) as a yellowish solid. ¹H NMR (400 MHz, CDCl₃) δ 7.71 (dd, J=8.7, 4.4 Hz, 2H), 7.52 (d, J=2.8 Hz, 1H), 7.20 (dd, J=8.8, 2.7 Hz, 1H), 7.01 (dd, J=8.7, 2.7 Hz, 1H), 6.83 (d, J=2.7 Hz, 1H), 4.36 (s, 2H), 3.91 (s, 3H), 3.86 (s, 3H).

### Step 3: Synthesis of 3,8-dihydroxy-6H-benzo[c]thiochromene 5,5-dioxide (62)

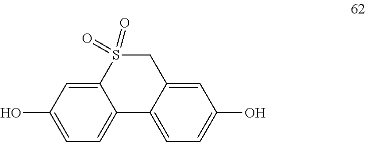 [SMILES: O=S1(=O)Cc2cc(O)ccc2-c2ccc(O)cc21]

BBr₃ (0.76 ml, 0.76 mmol) was added to a solution of 3,8-dimethoxy-6H-benzo[c]thiochromene 5,5-dioxide (55 mg, 0.19 mmol) in DCM 2 ml at −70° C. and the mixture was allowed to warm to room temperature overnight. TLC showed 2 spots. Methanol was added to the mixture at 0° C. was concentrated under vacuum and loaded on silica then purified by MPLC (SiO₂, MeOH/DCM 0% to 8%) to afford 3,8-dihydroxy-6H-benzo[c]thiochromene 5,5-dioxide (23 mg, 46%) as a yellowish solid. ¹H NMR (400 MHz, DMSO) δ 10.29 (s, 1H), 9.87 (s, 1H), 7.79 (d, J=8.8 Hz, 1H), 7.71 (d, J=8.6 Hz, 1H), 7.24 (d, J=2.6 Hz, 1H), 7.11 (dd, J=8.6, 2.7 Hz, 1H), 6.86 (dd, J=8.5, 2.6 Hz, 1H), 6.82 (d, J=2.6 Hz, 1H), 4.65 (s, 2H).

### Synthesis of 3,8-dihydroxy-6H-benzo[c]thiochromene 5-oxide (64)

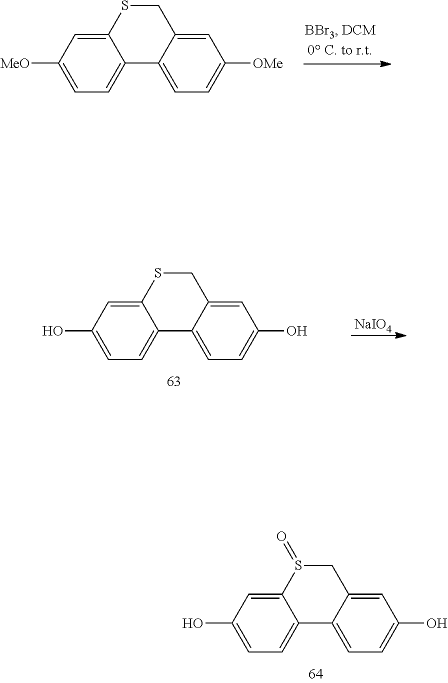 [SMILES: COc1ccc2c(c1)CSc1cc(OC)ccc1-2 ; Oc1ccc2c(c1)CSc1cc(O)ccc1-2 ; "NaIO4" ; O=S1Cc2cc(O)ccc2-c2ccc(O)cc21]

### Step 1: Synthesis of 6H-benzo[c]thiochromene-3 (8-diol (63)

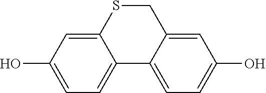 [SMILES: Oc1ccc2c(c1)CSc1cc(O)ccc1-2]

BBr₃ (0.81 ml, 0.81 mmol) was added at 0° C. to a solution of 3,8-dimethoxy-6H-benzo[c]thiochromene (70 mg, 0.27 mmol) in DCM 4 ml and allowed to warm to room temperature overnight. The reaction mixture was poured into methanol at 0° C. and stirred for 10 minutes then the solvent was evaporated under vacuum. The crude was filtered over a pad of silica to afford 6H-benzo[c]thiochromene-3,8-diol (40 mg, 64%) as a grey solid. R_{f}=0.75 (EtOAc/hexane 50/50). ¹H NMR (400 MHz, DMSO) δ 9.56 (s, 1H), 9.50 (s, 1H), 7.56 (d, J=8.6 Hz, 1H), 7.44 (d, J=8.5 Hz, 1H), 6.75-6.64 (m, 4H), 5.76 (s, 1H), 3.78 (s, 2H).

### Step 2: Synthesis of 3,8-dihydroxy-6H-benzo[c]thiochromene 5-oxide (64

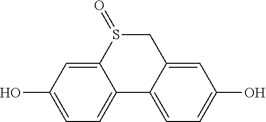 [SMILES: O=S1Cc2cc(O)ccc2-c2ccc(O)cc21]

A solution of NaIO4 (26 mg 0.12 mmol) in water 0.3 mL was added to a solution of 6H-benzo[c]thiochromene-3,8-diol (28 mg, 0.12 mmol) in MeOH 1.5 ml at r.t and the mixture stirred o.n. A precipitate was formed. TLC showed still starting material. Hence, 0.2 eq of NaIO₄ dissolved in water 0.2 mL was added and stirring continued; reaction not complete but stopped.

DCM was added to dissolve the precipitate and the crude was loaded on silica and purified by MPLC (SiO₂, MeOH/DCM 0% to 8%) to afford 3,8-dihydroxy-6H-benzo[c]thiochromene 5-oxide (16 mg, 53%) as a grey solid. R_{f}=0.3 (MeOH/DCM 5%). ¹H NMR (400 MHz, DMSO) δ 10.05 (s, 1H), 9.72 (s, 1H), 7.64 (dd, J=32.7, 8.5 Hz, 2H), 7.11 (d, J=2.6 Hz, 1H), 7.00 (dd, J=8.5, 2.6 Hz, 1H), 6.83 (d, J=6.7 Hz, 2H), 4.21 (dd, J=90.8, 14.2 Hz, 2H).

### 1) Ester “A” group with bicyclopentane substitution

### Synthesis of 3-hydroxy-8-(3-(hydroxymethyl)bicyclo[1.1.1]pentan-1-yl)-6H-benzo[c]chromen-6-one (65)

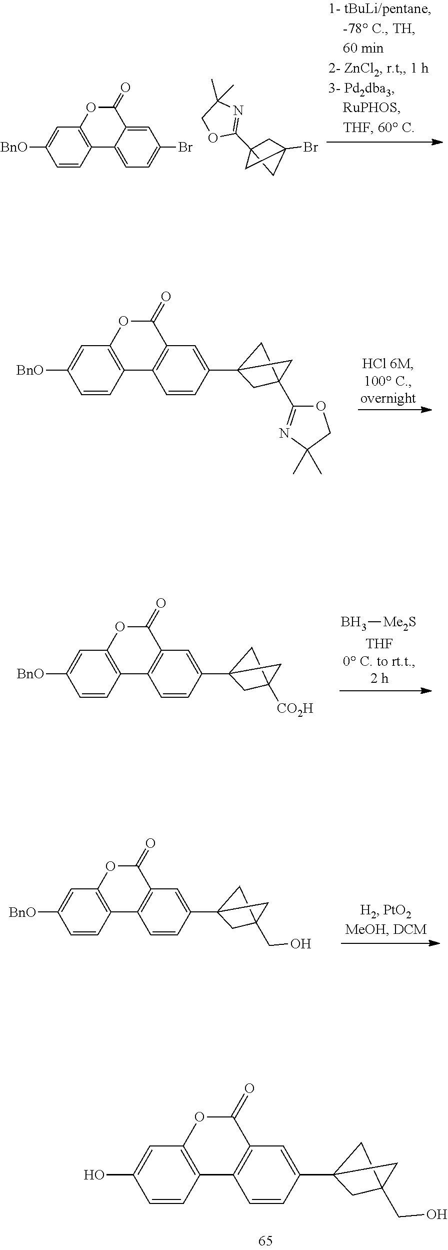

### Step 1: Synthesis of 3-(benzyloxy)-8-(3-(4,4-dimethyl-4,5-dihydrooxazol-2-yl)bicyclo[1.1.1]pentan-1-yl)-6H-benzo[c]chromen-6-one

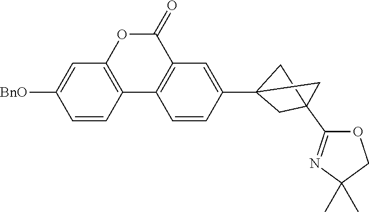 [SMILES: CC1(C)COC(C23CC(c4ccc5c(c4)c(=O)oc4cc(OCc6ccccc6)ccc45)(C2)C3)=N1]

To a cooled −78° C. solution of 2-(3-bromobicyclo[1.1.1]pentan-1-yl)-4,4-dimethyl-4,5-dihydrooxazole (192 mg, 0.788 mmol) in anhydrous 2.7 mL was added carefully dropwise tert-butyllithium (1.7 M in pentane, 0.95 ml, 1.63 mmol). The reaction mixture was stirred at −78° C. for 60 min. A solution of ZnCl2 [0.5M in THF] (1.78 ml, 0.89 mmol) was added dropwise. The reaction mixture was allowed to reach room temperature for 60 min. The resulting zincate solution was slowly added dropwise to a mixture of 3-(benzyloxy)-8-bromo-6H-benzo[c]chromen-6-one (200 mg, 0.525 mmol), RuPhos (49 mg, 0.105 mmol) and Tris(dibezylideneacetone)dipalladium (48 mg, 0.052 mmol) under N₂ atmosphere at room temperature. The reaction vessel was sealed and heated at 60° C. for 12 hrs. The reaction mixture was concentrated under reduced pressure and the resulting residue was absorbed on SiO2. Purification of the residue by MPLC (SiO₂, EtOAc/cyclohexane 0% to 20%) to give 3-(benzyloxy)-8-(3-(4,4-dimethyl-4,5-dihydrooxazol-2-yl)bicyclo[1.1.1]pentan-1-yl)-6H-benzo[c]chromen-6-one (90 mg, 0.19 mmol, 37%). ¹H NMR (500 MHz, CDCl₃) δ 8.18 (d, J=1.9 Hz, 1H), 7.94 (t, J=8.4 Hz, 2H), 7.64 (dd, J=8.2, 1.9 Hz, 1H), 7.45-7.35 (m, 5H), 6.99 (dd, J=8.8, 2.6 Hz, 1H), 6.93 (d, J=2.6 Hz, 1H), 5.14 (s, 2H), 3.97 (s, 2H), 2.40 (s, 6H), 1.31 (s, 6H).

### Step 2: Synthesis of 3-(3-(benzyloxy)-6-oxo-6H-benzo[c]chromen-8-yl)bicyclo[1.1.1]pentane-1-carboxylic acid

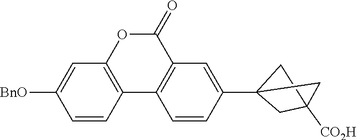 [SMILES: O=C(O)C12CC(c3ccc4c(c3)c(=O)oc3cc(OCc5ccccc5)ccc34)(C1)C2]

A suspension of 3-(benzyloxy)-8-(3-(4,4-dimethyl-4,5-dihydrooxazol-2-yl)bicyclo[1.1.1]pentan-1-yl)-6H-benzo[c]chromen-6-one (110 mg, 0.236 mmol) in 6M HCl was heated at 100° C. o.n. in a sealed tube. The reaction mixture was cooled down to r.t. then filtered and washed with water ad dried under high vacuum. the crude was purified by FC eluent MeOH/DCM 0% to 8% to give 3-(3-(benzyloxy)-6-oxo-6H-benzo[c]chromen-8-yl)bicyclo[1.1.1]pentane-1-carboxylic acid (70 mg, 72%) as a beige solid. LCMS no mass TLC/MS 413. R_{f}=0.5 (10% MeOH/DCM). ¹H NMR (400 MHz, DMSO) δ 12.45 (s, 1H), 8.29 (dd, J=13.3, 8.6 Hz, 2H), 8.00 (d, J=1.9 Hz, 1H), 7.81 (dd, J=8.3, 1.9 Hz, 1H), 7.53-7.32 (m, 5H), 7.13-7.06 (m, 2H), 5.24 (s, 2H), 2.33 (s, 6H).

### Step 3: Synthesis of 3-(benzyloxy)-8-(3-(hydroxymethyl)bicyclo[1.1.1]pentan-1-yl)-6H-benzo[c]chromen-6-one

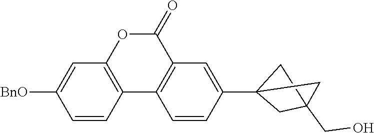 [SMILES: O=c1oc2cc(OCc3ccccc3)ccc2c2ccc(C34CC(CO)(C3)C4)cc12]

Borane dimethyl sulfide complex (0.22 ml, 0.44 mmol, 2 M in THF, 3.0 eq) was added to a solution of (3-(3-(benzyloxy)-6-oxo-6H-benzo[c]chromen-8-yl)bicyclo[1.1.1]pentane-1-carboxylic acid (60 mg, 0.15 mmol, 1.0 eq) at 0° C. in THF 2 ml and stirring continued for 2 h from 0° C. to rt. MeOH was added and the crude was loaded on silica and purified by FC eluent MeOH/DCM 0% to 5% to give 3-(benzyloxy)-8-(3-(hydroxymethyl)bicyclo[1.1.1]pentan-1-yl)-6H-benzo[c]chromen-6-one (47 mg, 0.12 mmol, 81%) as a beige solid. R_{f}=0.6 (MeOH/DCM 5%). ¹H NMR (400 MHz, CDCl₃) δ 8.19 (d, J=1.9 Hz, 1H), 7.94 (dd, J=8.6, 3.3 Hz, 2H), 7.65 (dd, J=8.3, 1.9 Hz, 1H), 7.42 (dtdd, J=14.5, 8.7, 6.9, 1.8 Hz, 5H), 6.99 (dd, J=8.8, 2.6 Hz, 1H), 6.94 (d, J=2.5 Hz, 1H), 5.30 (s, 1H), 5.14 (s, 2H), 3.74 (s, 2H), 2.07 (s, 6H).

### Step 4: Synthesis of 3-hydroxy-8-(3-(hydroxymethyl)bicyclo[1.1.1]pentan-1-yl)-6H-benzo[c]chromen-6-one

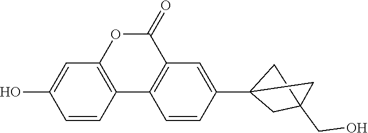 [SMILES: O=c1oc2cc(O)ccc2c2ccc(C34CC(CO)(C3)C4)cc12]

3-(benzyloxy)-8-(3-(hydroxymethyl)bicyclo[1.1.1]pentan-1-yl)-6H-benzo[c]chromen-6-one (45 mg, 0.11 mmol) was dissolved in MeOH 3 ml and DCM 1 ml. PtO₂ (6.4 mg, 0.023 mmol) was added and the mixture was hydrogenated under atmospheric pressure for 5 h. The reaction mixture was filtered over a pad of celite and concentrated under vacuum. The crude was purified by FC MeOH/DCM 0% to 10% to give 3-hydroxy-8-(3-(hydroxymethyl)bicyclo[1.1.1]pentan-1-yl)-6H-benzo[c]chromen-6-one (1.5 mg, 0.069 mmol, 62%) as a white solid. R_{f}=0.3 (EtOAc/hexane 50%); R_{f}=0.5 (MeOH/DCM 10%). ¹H NMR (400 MHz, DMSO) δ 10.31 (s, 1H), 8.21 (d, J=8.3 Hz, 1H), 8.14 (d, J=8.8 Hz, 1H), 7.94 (d, J=1.9 Hz, 1H), 7.74 (dd, J=8.2, 1.9 Hz, 1H), 6.84 (dd, J=8.7, 2.4 Hz, 1H), 6.75 (d, J=2.4 Hz, 1H), 4.58 (t, J=5.5 Hz, 1H), 3.48 (d, J=5.6 Hz, 2H), 1.96 (s, 6H).

### 2. Synthesis of 7-membered Urolithin A analogues

### A) Lactones and ethers “A” group analogues

### Synthesis of 3,9-dihydroxydibenzo[c,e]oxepin-5(7H)-one (66)

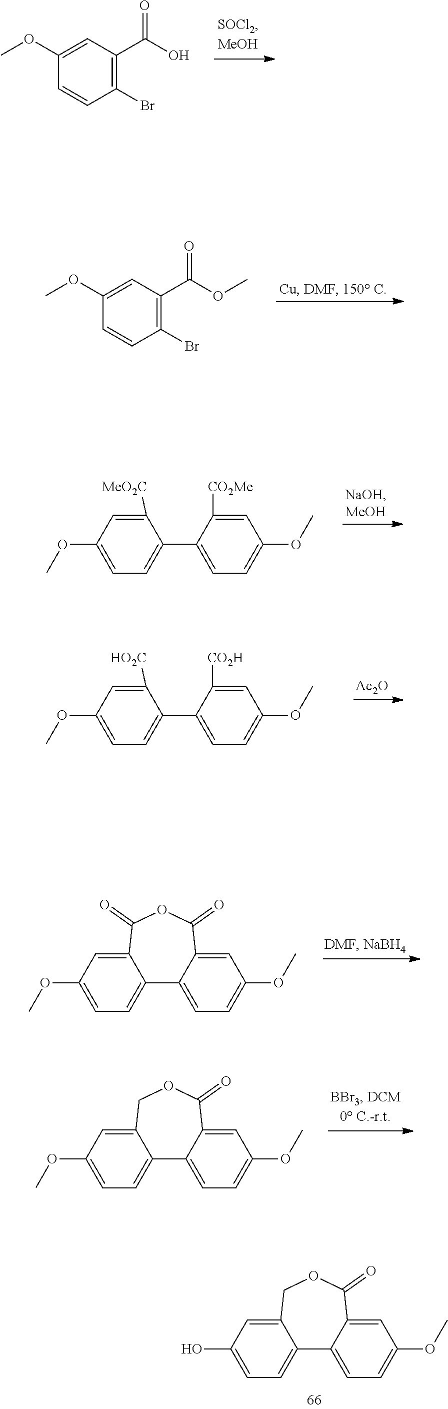

### Step 1: Synthesis of 2-bromo-5-methoxybenzoate

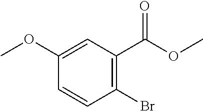

2-bromo-5-methoxybenzoic acid (11.6 g, 50.0 mmol, 1.00 eq.) was dissolved in MeOH (250 mL) and the resulting solution was cooled down to 0° C. in an ice-bath. Stirring at 0° C. was continued for 10 min and then SOCl₂ (17.8 g, 150 mmol, 3.00 eq.) was added dropwise via a dropping funnel. The reaction was allowed to warm up to r.t. and as soon as no more starting material could be observed (overnight stirring) all the volatiles were evaporated and the crude residue taken up in diethyl ether and filtered through silica. The filtrate was concentrated under vacuo to afford pure methyl 2-bromo-5-methoxybenzoate (12.3 g, 49.9 mmol, 99%) as a colorless oil that solidified upon storage. NMR matched precedent literature.

### Step 2: Synthesis of dimethyl 4,4′-dimethoxy-[1,1′-biphenyl]-2,2′-dicarboxylate

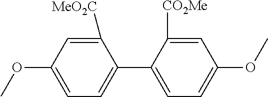

2-bromo-5-methoxybenzoate (12.3 g, 50 mmol, 1.00 eq.) was dissolved in DMF (60 mL) and copper powder (12.7 g, 200 mmol, 4.00 eq.) was added to the solution in one portion. Subsequently the reaction mixture was heated to 150° C. overnight. After overnight stirring the reaction was allowed to cool to r.t. and diluted with a copious amount of water and extracted with diethyl ether (3×100 mL). The combined organic layers were washed with water and brine, dried over Na₂SO₄, filtered through silica and concentrated in vacuo. The crude product was purified by flash column chromatography (SiO₂, 330 g, EtOAc in Hex 0-30%) to give dimethyl 4,4′-dimethoxy-[1,1′-biphenyl]-2,2′-dicarboxylate (7.5 g, 23 mmol, 91%) as a colorless oil. ¹H NMR (400 MHz, CDCl₃) δ 7.49 (d, J=2.7 Hz, 2H), 7.11 (d, J=8.3 Hz, 2H), 7.06 (dd, J=8.4, 2.7 Hz, 2H), 3.88 (s, 6H), 3.63 (s, 6H).

### Step 3: Synthesis of 4,4′-dimethoxy-[1,1′-biphenyl]-2,2′-dicarboxylic acid

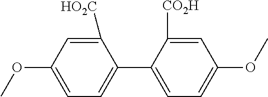

dimethyl 4,4′-dimethoxy-[1,1′-biphenyl]-2,2′-dicarboxylate (7.5 g, 23 mmol, 1.0 eq.) was dissolved in MeOH (90 mL) and a 2M aq. solution of NaOH (57 mL, 110 mmol, 5.0 eq.) was added dropwise via an addition funnel. The reaction was refluxed over the weekend before being allowed to cool to r.t. upon which the reaction mixture was concentrated under vacuo. The remaining organic layer was slightly diluted with water and washed with DCM to remove all organic impurities. The layers were separated and the aqueous layer was transferred into a conical flask and with stirring acidified to pH1 with 2M KHSO₄. Stirring was continued for 30 min and the formed precipitate was filtered, washed with water and dried under high vacuum to obtain 4,4′-dimethoxy-[1,1′-biphenyl]-2,2′-dicarboxylic acid (6.64 g, 22.0 mmol, 97%) as a free-flowing white solid. ¹H NMR (400 MHz, DMSO) δ 12.43 (s, 2H), 7.33 (d, J=2.6 Hz, 2H), 7.14-7.01 (m, 4H), 3.82 (s, 6H).

### Step 4: Synthesis of 3,9-dimethoxydibenzo[c,e]oxepine-5,7-dione

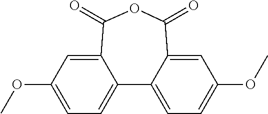

4,4′-dimethoxy-[1,1′-biphenyl]-2,2′-dicarboxylic acid (2.60 g, 10.1 mmol, 1.0 eq.) was suspended in Ac₂O (50 mL) and the suspension was stirred overnight. The reaction was monitored by LCMS and after overnight stirring the starting material completely disappeared. Then the reaction mixture was filtered and washed with diethyl ether to facilitate drying. The filter cake was dried under high vacuum to yield 3,9-dimethoxydibenzo[c,e]oxepine-5,7-dione (2.87 g, 10.1 mmol, 99%). The NMR matched with the one reported in the literature.

### Step 5: Synthesis of 3,9-dimethoxydibenzo[c,e]oxepin-5(7H)-one

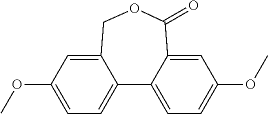

3,9-dimethoxydibenzo[c,e]oxepine-5,7-dione (150 mg, 0.530 mmol, 1.0 eq.) was suspended in DMF (5 mL) and cooled to 0° C. before sodium borohydride (20 mg, 0.53 mmol, 1.0 eq.) was added slowly. After two hours the reaction mixture was poured into aq. HCl (6M, 5 mL) which was then subsequently diluted with water (10 mL) and stirred overnight. The product was precipitated overnight and was filtered before being taken up in DCM (25 mL) and washed with water (3×10 mL). The organic layer was dried over anhydrous Na₂SO₄, filtered, concentrated in vacuo, filtered through basic alumina with DCM and dried to afford 3,9-dimethoxydibenzo[c,e]oxepin-5(7H)-one (85 mg, 0.31 mmol, 60%) as a white solid. ¹H NMR (400 MHz, CDCl₃) δ 7.52 (d, J=8.6 Hz, 1H), 7.50-7.44 (m, 2H), 7.19 (dd, J=8.7, 2.8 Hz, 1H), 7.05 (dd, J=8.6, 2.7 Hz, 1H), 6.97 (d, J=2.7 Hz, 1H), 4.98 (d, J=28.5 Hz, 2H), 3.90 (s, 3H), 3.87 (s, 3H).

### Step 5: Synthesis of 3,9-dihydroxydibenzo[c,e]oxepin-5(7H)-one

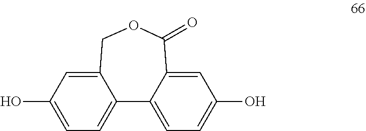 [SMILES: O=C1OCc2cc(O)ccc2-c2ccc(O)cc21]

3,9-dimethoxydibenzo[c,e]oxepin-5(7H)-one (75 mg, 0.28 mmol, 1.0 eq.) was dissolved in DCM (6 mL) and cooled down to 0° C. in an ice-bath and stirring was continued for 5 min. Then BBr₃ (0.83 ml, 1M in DCM, 0.83 mmol, 3.00 eq.) was added dropwise to the reaction mixture. Upon complete addition the mixture was left in the ice bath and was allowed to warm to r.t. over the course of 2 h. When no more starting material could be observed the reaction mixture was dropwise added into 0° C. cold methanol (10 mL) and stirred for an additional 10 min. Then the mixture was concentrated and loaded on silica to be purified by flash column chromatography (SiO₂, 12 g, MeOH in DCM 0-5%) 3,9-dihydroxydibenzo[c,e]oxepin-5(7H)-one (19 mg, 0.8 mmol, 28%) as white solid. ¹H NMR (400 MHz, MeOD) δ 7.49 (dd, J=8.5, 7.3 Hz, 2H), 7.28 (d, J=2.7 Hz, 1H), 7.14 (dd, J=8.6, 2.7 Hz, 1H), 6.97 (dd, J=8.4, 2.6 Hz, 1H), 6.93 (d, J=2.6 Hz, 1H), 4.96 (d, J=19.5 Hz, 2H).

### Synthesis of 5,7-dihydrodibenzo[c,e]oxepine-3,9-diol (67)

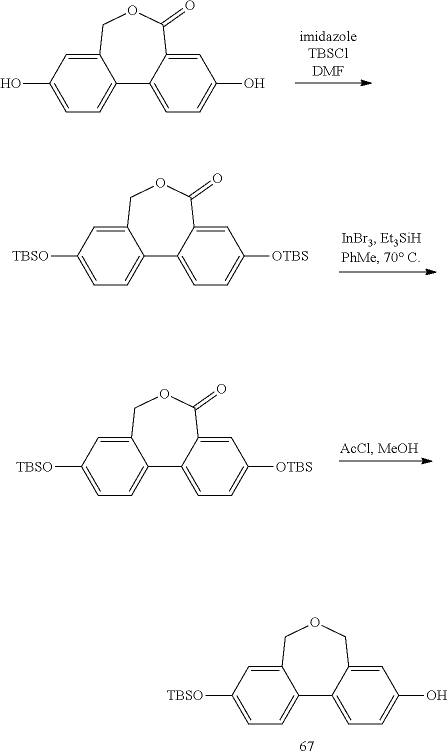 [SMILES: O=C1OCc2cc(O)ccc2-c2ccc(O)cc21 ; CC(C)(C)[Si](C)(C)Oc1ccc2c(c1)COC(=O)c1cc(O[Si](C)(C)C(C)(C)C)ccc1-2 ; CC(C)(C)[Si](C)(C)Oc1ccc2c(c1)COC(=O)c1cc(O[Si](C)(C)C(C)(C)C)ccc1-2 ; CC(C)(C)[Si](C)(C)Oc1ccc2c(c1)COCc1cc(O)ccc1-2]

### Step 1: Synthesis of 3,9-bis((tert-butyldimethylsilyl)oxy)dibenzo[c,e]oxepin-5(7H)-one

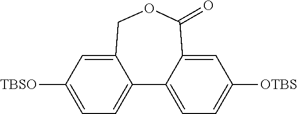 [SMILES: CC(C)(C)[Si](C)(C)Oc1ccc2c(c1)COC(=O)c1cc(O[Si](C)(C)C(C)(C)C)ccc1-2]

TBSCl (174 mg, 1.15 mmol, 2.2 eq.) was dissolved in DCM (9 mL) and the resulting solution was cooled to 0° C. in an ice-bath and stirred for 5 min. Then imidazole (89 mg, 1.3 mmol, 2.5 eq.) was slowly added in portions and upon complete addition stirring was continued for 15 min. Subsequently 3,9-dihydroxydibenzo[c,e]oxepin-5(7H)-one (127 mg, 0.520 mmol, 1.0 eq.) was added to the reaction mixture which became heterogenous upon addition of the substrate. Therefore DMF (1 mL) was added in order to homogenize the mixture. Stirring at r.t. was continued overnight before the DCM was removed at the rotary evaporator and the remaining DMF solution was quenched with copious amounts of water and extracted with diethyl ether (3×10 mL). The combined organic layers were washed with water and brine, dried over anhydrous Na₂SO₄, filtered and concentrated to give the crude product which was further purified by MPLC (SiO₂, 40 g, EtOAc in Hex 0-20%) to afford 3,9-bis((tert-butyldimethylsilyl)oxy)dibenzo[c,e]oxepin-5(7H)-one (199 mg, 0.42 mmol 82%) as a white solid. ¹H NMR (400 MHz, CDCl₃) δ 7.47-7.38 (m, 3H), 7.10 (dd, J=8.6, 2.6 Hz, 1H), 6.97 (dd, J=8.4, 2.6 Hz, 1H), 6.90 (d, J=2.6 Hz, 1H), 4.88 (d, 2H), 1.01 (d, J=1.9 Hz, 18H), 0.25 (d, J=8.8 Hz, 12H).

### Step 2: Synthesis of 3,9-bis((tert-butyldimethylsilyl)oxy)-5,7-dihydrodibenzo[c,e]oxepine

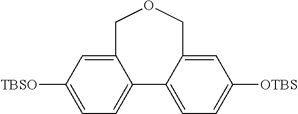 [SMILES: CC(C)(C)[Si](C)(C)Oc1ccc2c(c1)COCc1cc(O[Si](C)(C)C(C)(C)C)ccc1-2]

3,9-bis((tert-butyldimethylsilyl)oxy)dibenzo[c,e]oxepin-5(7H)-one (200 mg, 0.430 mmol, 1.0 eq.) was dissolved in toluene (5 mL) and Et₃SiH (0.27 ml, 1.7 mmol, 4.0 eq.) was added in one portion. The reaction mixture was heated to 70° C. in a pre-heated oil-bath. Upon stirring for 5 min at 70° C. InBr₃ (15 mg, 0.04 mmol, 0.10 eq.) was added in one portion. A quick color change to orange as well as the evolution of gas could be observed and stirring was continued for 1 h und the TLC did not show any more starting material. The reaction mixture was cooled down, filtered and the precipitate washed with DCM. The filtrate was loaded on silica and the crude was purified by flash column chromatography (SiO₂, 25 g, DCM in Hex 0-10%) to yield 3,9-bis((tert-butyldimethylsilyl)oxy)-5,7-dihydrodibenzo[c,e]oxepine (194 mg, 0.430 mmol, 99%) as a white solid. ¹H NMR (400 MHz, CDCl³) δ 7.36 (d, J=8.3 Hz, 2H), 6.94 (dd, J=8.3, 2.5 Hz, 2H), 6.90 (d, J=2.5 Hz, 2H), 4.31 (s, 4H), 1.01 (s, 18H), 0.24 (s, 12H).

### Step 3: Synthesis of 5,7-dihydrodibenzo[c,e]oxepine-3,9-diol

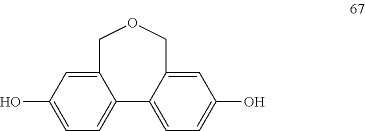 [SMILES: Oc1ccc2c(c1)COCc1cc(O)ccc1-2]

3,9-bis((tert-butyldimethylsilyl)oxy)-5,7-dihydrodibenzo[c,e]oxepine (194 mg, 0.430 mmol, 1.0 eq.) was dissolved in MeOH (12 mL) and the reaction mixture was cooled to 0° C. and AcCl (167 mg, 2.12 mmol, 5.0 eq.) were added dropwise via syringe. Upon complete addition, the reaction mixture was allowed to r.t. and stirring was continued over the weekend. The reaction was quenched with water and extracted into diethyl ether (3×15 mL) and the combined organic layers were washed with NaHCO₃ and brine, dried over Na₂SO₄ and filtered through silica with diethyl ether washings and then concentrated to give pure 5,7-dihydrodibenzo[c,e]oxepine-3,9-diol (71 mg, 0.31 mmol, 73%) as a white solid. ¹H NMR (400 MHz, DMSO) δ 9.55 (s, 2H), 7.30 (d, J=8.2 Hz, 2H), 6.87 (dd, J=8.2, 2.6 Hz, 2H), 6.84 (d, J=2.5 Hz, 2H), 4.13 (s, 4H).

### B) Amine “A” group analogues

### Synthesis of 6-methyl-6,7-dihydro-5H-dibenzo[c,e]azepine-3,9-diol (68)

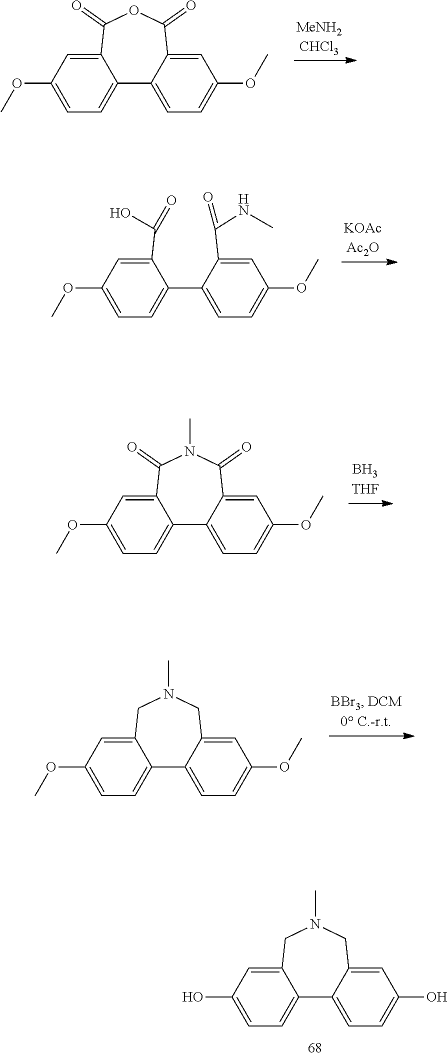

### Step 1: Synthesis of 4,4′-dimethoxy-2′-(methylcarbamoyl)-[1,1′-biphenyl]-2-carboxylic acid

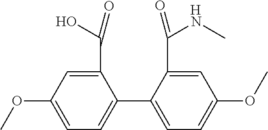

3,9-dimethoxydibenzo[c,e]oxepine-5,7-dione (569 mg, 2.00 mmol, 1.0 eq.) was dissolved in CHCl₃ (20 mL) and to the resulting solution a 2M solution of MeNH₂ (1.20 mL, 2.40 mmol, 1.2 eq.) was added in one portion. Upon addition of the MeNH₂ a precipitate formed and the complete disappearance of starting material could be observed via LCMS. The precipitate was filtered over a glass frit (Por.4) and the filter residue was dried under vacuo to afford pure 4,4′-dimethoxy-2′-(methylcarbamoyl)-[1,1′-biphenyl]-2-carboxylic acid (631 mg, 2.00 mmol, 99%) as a light brown solid. LCMS showed clean product which was carried on further to the next step.

### Step 2: Synthesis of 3,9-dimethoxy-6-methyl-5H-dibenzo[c,e]azepine-5,7(6H)-dione

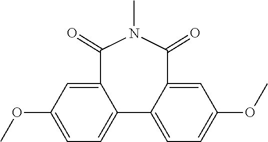

4,4′-dimethoxy-2′-(methylcarbamoyl)-[1,1′-biphenyl]-2-carboxylic acid (631 mg, 2.00 mmol, 1.00 eq.) were suspended in Ac₂O (20 mL) and KOAc (393 mg, 4.00 mmol, 2.00 eq.) were added in one portion. The reaction was stirred overnight and LCMS showed the complete conversion of starting material, therefore the suspension was filtered and the filter residue was dried under high vacuum to afford 3,9-dimethoxy-6-methyl-5H-dibenzo[c,e]azepine-5,7(6H)-dione (595 mg, 2.00 mmol, 99%). ¹H NMR (400 MHz, CDCl₃) δ 7.51 (d, J=8.7 Hz, 2H), 7.38 (d, J=2.8 Hz, 2H), 7.16 (dd, J=8.7, 2.8 Hz, 2H), 3.90 (s, 6H), 3.54 (s, 3H).

### Step 3: Synthesis of 3,9-dimethoxy-6-methyl-6,7-dihydro-5H-dibenzo[c,e]azepine

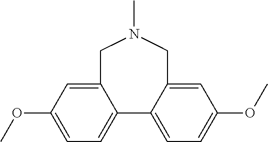

3,9-dimethoxy-6-methyl-5H-dibenzo[c,e]azepine-5,7(6H)-dione (541 mg, 1.82 mmol, 1.0 eq.) was suspended in THF (15 mL) and at r.t. BH₃*THF (7.28 mL, 7.28 mmol, 1M, 4.0 eq.) was added dropwise over the course of 5 min. Upon complete addition, the reaction was heated to reflux and stirred overnight. Following the reaction was quenched with MeOH (200 mL) and stirring at 50° C. was continued for 30 min. Subsequently the volatiles were evaporated and the crude material was purified by MPLC (SiO₂, 40 g, MeOH in EtOAc 0-50%) to obtain 3,9-dimethoxy-6-methyl-6,7-dihydro-5H-dibenzo[c,e]azepine (485 mg, 1.80 mmol, 99%) as an orange-brown solid. ¹H NMR (400 MHz, CDCl₃) δ 7.38 (d, J=8.4 Hz, 2H), 6.97 (dd, J=8.4, 2.7 Hz, 2H), 6.91 (d, J=2.7 Hz, 2H), 3.86 (s, 6H), 3.37 (s, 4H), 2.48 (s, 3H).

### Step 4: Synthesis of 6-methyl-6,7-dihydro-5H-dibenzo[c,e]azepine-3,9-diol

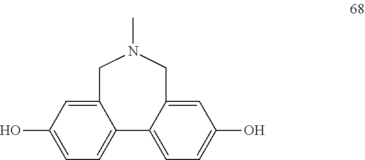 [SMILES: CN1Cc2cc(O)ccc2-c2ccc(O)cc2C1]

3,9-dimethoxy-6-methyl-6,7-dihydro-5H-dibenzo[c,e]azepine (376 mg, 1.40 mmol, 1.0 eq.) was dissolved in DCM (10 mL) and cooled down to 0° C. in an ice-bath and stirring was continued for 5 min. Then BBr₃ (6.28 ml, 1M in DCM, 6.28 mmol, 4.5 eq.) was added dropwise to the reaction mixture. Upon complete addition the mixture was left in the ice bath and was allowed to warm to r.t. over the course of 2 h. When no more starting material could be observed the reaction mixture was dropwise added into 0° C. cold methanol (10 mL) and stirred for an additional 10 min. Then the mixture was concentrated and loaded on silica to be purified by flash column chromatography (SiO₂, 40 g, MeOH in DCM 0-5%) 6-methyl-6,7-dihydro-5H-dibenzo[c,e]azepine-3,9-diol (190 mg, 0.790 mmol, 56%) as pale orange solid. ¹H NMR (400 MHz, DMSO) δ 10.92-10.62 (m, 2H), 7.37 (d, J=8.2 Hz, 2H), 7.10-6.93 (m, 4H), 3.16 (s, 4H), 2.83 (d, J=4.6 Hz, 3H).

### Synthesis of 2,2′-bis(bromomethyl)-4,4′-dimethoxy-1,1′-biphenyl as a common intermediate

### Step 1: Synthesis of (4,4′-dimethoxy-[1,1′-biphenyl]-2,2′-diyl)dimethanol

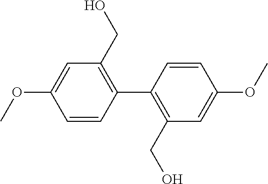

LiAlH₄ (251 mg, 6.61 mmol) was added carefully to a solution of 4,4′-dimethoxy-[1,1′-biphenyl]-2,2′-dicarboxylic acid (previously described above) (1.00 g, 3.30 mmol) in THF (8 ml) at 0° c. then refluxed for 4 h (reaction monitored by TLC). After Fieser workup 850 mg of (4,4′-dimethoxy-[1,1′-biphenyl]-2,2′-diyl)dimethanol (810 mg, 2.90 mmol, 89%) as a white solid. ¹H NMR (400 MHz, CDCl₃) δ 7.08-7.03 (m, 4H), 6.87 (dd, J=8.3, 2.8 Hz, 2H), 4.40-4.28 (m, 4H), 3.86 (s, 6H), 2.20 (s, 2H).

### Step 2: Synthesis of 2,2′-bis(bromomethyl)-4,4′-dimethoxy-1,1′-biphenyl

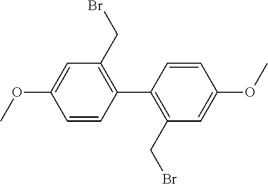

To a solution of (4,4′-dimethoxy-[1,1′-biphenyl]-2,2′-diyl)dimethanol (0.800 g, 2.92 mmol) and CBr₄ (4.84 g, 14.6 mmol) in CH₂C₁₂ (40 mL) cooled to 0° C. under an argon atmosphere was added portion-wise a solution of PPh₃ (3.06 g, 11.7 mmol) in CH2Cl2 (20 mL). The reaction was stirred at room temp for 48 hrs, then concentrated and the crude product purified by MPLC on silica gel (EtOAc/hexane: 0% to 10%) to give 2,2′-bis(bromomethyl)-4,4′-dimethoxy-1,1′-biphenyl (0.88 g, 2.20 mmol, 75%) as colorless oil. R_{f}=0.5 (EtOAc/cyclohexane 10%). ¹H NMR (400 MHz, CDCl₃) δ 7.17 (d, J=8.4 Hz, 2H), 7.05 (d, J=2.7 Hz, 2H), 6.91 (dd, J=8.4, 2.7 Hz, 2H), 4.31 (d, J=10.0 Hz, 2H), 4.17 (d, J=10.0 Hz, 2H), 3.87 (s, 6H).

### Synthesis of 6-cyclobutyl-6,7-dihydro-5H-dibenzo[c,e]azepine-3,9-diol 69)

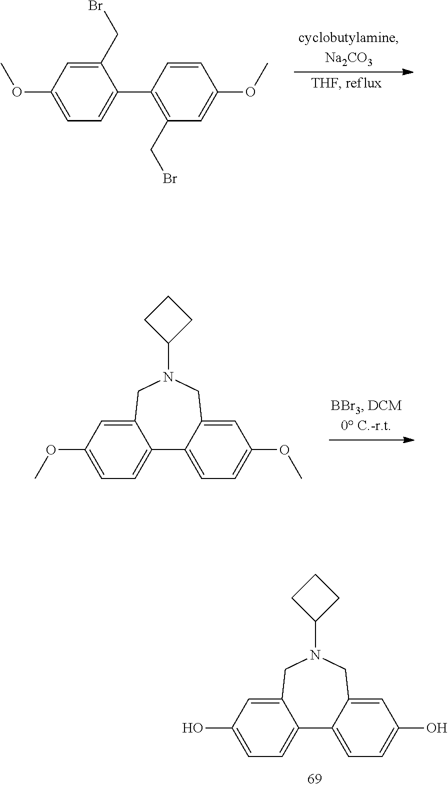

### Step 1: Synthesis of 6-cyclobutyl-3,9-dimethoxy-6,7-dihydro-5H-dibenzo[c,e]azepine

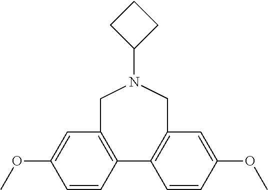

Cyclobutylamine (28 mg, 0.39 mmol) was added to a suspension of 2,2′-bis(bromomethyl)-4,4′-dimethoxy-1,1′-biphenyl (130 mg, 0.325 mmol) and sodium carbonate (138 mg, 130 mmol) in THF 2 mL and the mixture was refluxed for 3 h in THF. The reaction mixture was filtered off and the solvent was removed under vacuum to give 6-cyclobutyl-3,9-dimethoxy-6,7-dihydro-5H-dibenzo[c,e]azepine (100 mg, 0.323 mmol, 99%) as colorless oil. R_{f}=0.3 (EtOAc). ¹H NMR (400 MHz, CDCl₃) δ 7.36 (d, J=8.4 Hz, 2H), 6.95 (dd, J=8.4, 2.7 Hz, 2H), 6.87 (d, J=2.7 Hz, 2H), 3.86 (s, 6H), 3.28 (s, 4H), 3.12 (p, J=8.0 Hz, 1H), 2.21-2.12 (m, 2H), 2.05 (d, J=9.6 Hz, 2H), 1.82-1.65 (m, 2H).

### Step 2: Synthesis of 6-cyclobutyl-6,7-dihydro-5H-dibenzo[c,e]azepine-3,9-diol

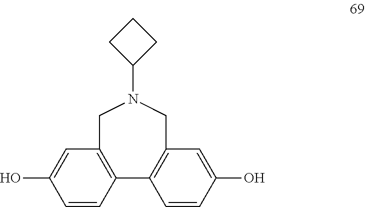 [SMILES: Oc1ccc2c(c1)CN(C1CCC1)Cc1cc(O)ccc1-2]

BBr₃ (0.87 ml, 0.87 mmol, 1.0M in DCM) was added to a solution of 6-cyclobutyl-3,9-dimethoxy-6,7-dihydro-5H-dibenzo[c,e]azepine (90 mg, 0.29 mmol) in dry DCM 3 ml at 0° c. and stirring continued overnight. Methanol 2 ml was added at 0° c. and the mixture was evaporated under vacuum. The crude product purified by flash chromatography on silica gel (Methanol/DCM: 0% to 10%) to give 6-cyclobutyl-6,7-dihydro-5H-dibenzo[c,e]azepine-3,9-diol hydrobromide (35 mg, 0.97 mmol, 33%) as a beige solid. R_{f}=0.3 (MeOH/DCM 8%). MS (ESI+): m/z=282. ¹H NMR (400 MHz, DMSO) δ 10.72 (s, 1H), 9.85 (s, 2H), 7.37 (d, J=9.0 Hz, 2H), 6.99 (dd, J=5.9, 2.8 Hz, 4H), 3.89 (s, 2H), 3.74 (d, J=8.7 Hz, 1H), 3.51 (s, 2H), 2.37-2.21 (m, 4H), 1.75 (dt, J=28.5, 10.0 Hz, 2H).

### Synthesis of 6-isopropyl-6,7-dihydro-5H-dibenzo[c,e]azepine-3,9-diol (70)

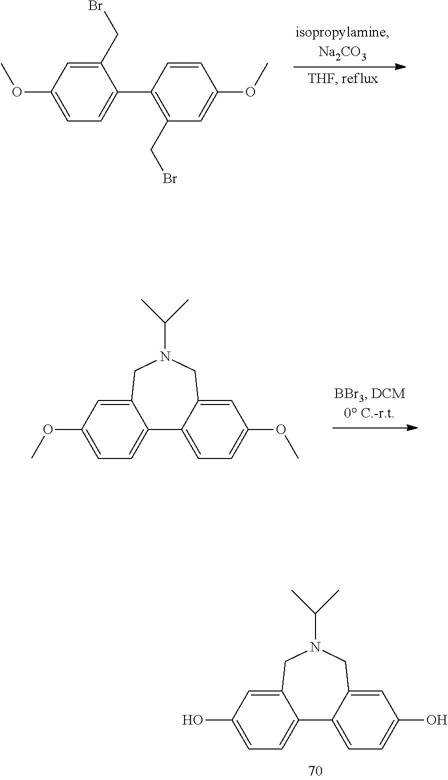

### Step 1: Synthesis 6-isopropyl-3,9-dimethoxy-6,7-dihydro-5H-dibenzo[c.e]azepine

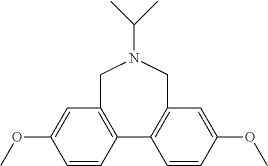

Isopropylamine (27 mg, 0.45 mmol) was added to a suspension of 2,2′-bis(bromomethyl)-4,4′-dimethoxy-1,1′-biphenyl (150 mg, 0.375 mmol) and sodium carbonate (159 mg, 1.50 mmol) in THF (2 mL) and the mixture was refluxed for 3 h in THF. The reaction mixture was filtered off and the solvent was removed under vacuum to 6-isopropyl-3,9-dimethoxy-6,7-dihydro-5H-dibenzo[c,e]azepine (110 mg, 0.323 mmol, 99%) as colourless oil. ¹H NMR (400 MHz, CDCl₃) δ 7.44-7.33 (m, 2H), 6.99 (d, J=7.3 Hz, 4H), 3.87 (s, 6H), 3.65 (s, 4H), 3.14-3.00 (m, 1H), 1.38 (d, J=6.4 Hz, 6H).

### Step 2: Synthesis of 6-isopropyl-6,7-dihydro-5H-dibenzo[c,e]azepine-3,9-diol

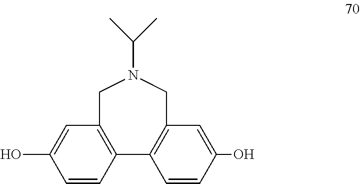 [SMILES: CC(C)N1Cc2cc(O)ccc2-c2ccc(O)cc2C1]

BBr₃ (1.87 ml, 1.87 mmol, 1.0M in DCM) was added to a solution of 6-isopropyl-3,9-dimethoxy-6,7-dihydro-5H-dibenzo[c,e]azepine (111 mg, 0.370 mmol) in dry DCM 3 ml at 0° C. and stirring continued overnight. Methanol 2 ml was added at 0° C. and the mixture was evaporated under vacuum. The crude product purified by flash chromatography on silica gel (Methanol/DCM: 0% to 10%) to give 6-6-isopropyl-6,7-dihydro-5H-dibenzo[c,e]azepine-3,9-diol (35 mg, 0.97 mmol, 35%) as a beige solid. R_{f}=0.3 (MeOH/DCM 8%). ¹H NMR (400 MHz, DMSO) δ 10.19 (s, 1H), 9.84 (s, 2H), 7.37 (d, J=8.3 Hz, 2H), 7.05 (d, J=2.6 Hz, 2H), 6.99 (dd, J=8.4, 2.5 Hz, 2H), 3.92 (s, J=4.3 Hz, 4H), 3.63-3.51 (m, 1H), 1.40 (d, J=6.5 Hz, 6H).

### C) Imide “A” group analogues

### Synthesis of 3,9-dihydroxy-5H-dibenzo[c,e]azepine-5,7(6H)-dione (71)

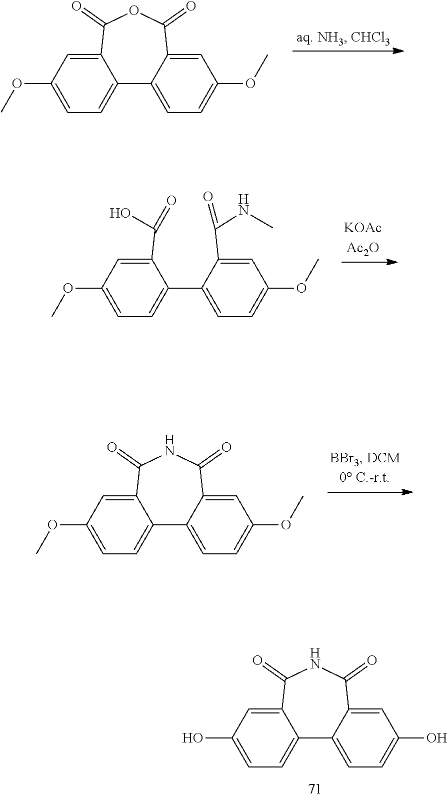

### Step 1: Synthesis of 2′-carbamoyl-4,4′-dimethoxy-[1,1′-biphenyl]-2-carboxylic acid

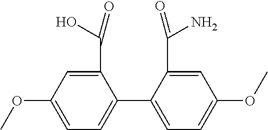

3,9-dimethoxydibenzo[c,e]oxepine-5,7-dione (100 mg, 0.350 mmol, 1.0 eq.) was suspended in 25% aq. NH₃ solution (0.70 mL, 0.42 mmol, 1.2 eq.) for 30 min until the complete disappearance of the starting material was confirmed by LCMS (too polar to be monitored by TLC). The reaction mixture was filtered over a glass frit (Por.4) and the filter residue was dried under vacuo to afford pure 2′-carbamoyl-4,4′-dimethoxy-[1,1′-biphenyl]-2-carboxylic acid (106 mg, 0.350 mmol, 99%) as a white solid. LCMS showed clean product after filtration and the product was used without further purification in the next step.

### Step 2: Synthesis of 3,9-dimethoxy-5H-dibenzo[c,e]azepine-5,7(6H)-dione

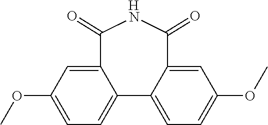

2′-carbamoyl-4,4′-dimethoxy-[1,1′-biphenyl]-2-carboxylic acid (106 mg, 0.350 mmol, 1.0 eq.) was suspended in Ac₂O (4 mL) and KOAc (69 mg, 0.70 mmol, 2.0 eq.) was added in one portion. The reaction mixture was stirred at r.t. overnight before being filtered over a small glass frit (Por.4). The precipitate was dried under vacuo to afford 3,9-dimethoxy-5H-dibenzo[c,e]azepine-5,7(6H)-dione (65 mg, 0.23 mmol, 65%) as a white solid. ¹H NMR (400 MHz, DMSO) δ 11.69 (s, 1H), 7.71 (dd, J=8.7, 1.5 Hz, 2H), 7.40-7.36 (m, 2H), 7.31 (dt, J=8.8, 2.4 Hz, 2H), 3.86 (s, 6H).

### Step 3: Synthesis of 3,9-dihydroxy-5H-dibenzo[c,e]azepine-5,7(6H)-dione

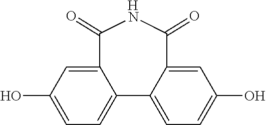

3,9-dimethoxy-5H-dibenzo[c,e]azepine-5,7(6H)-dione (100 mg, 0.350 mmol, 1.0 eq.) was dissolved in DCM (2 mL) and cooled down to 0° C. in an ice-bath and stirring was continued for 5 min. Then BBr₃ (1.41 ml, 1M in DCM, 1.41 mmol, 4.0 eq.) was added dropwise to the reaction mixture. Upon complete addition the mixture was left in the ice bath and was allowed to warm to r.t. over the course of 2 h. When no more starting material could be observed the reaction mixture was dropwise added into 0° C. cold methanol (10 mL) and stirred for an additional 10 min. Then the mixture was concentrated and loaded on silica to be purified by flash column chromatography (SiO₂, 12 g, MeOH in DCM 0-5%) 3,9-dihydroxy-5H-dibenzo[c,e]azepine-5,7(6H)-dione (56 mg, 0.22 mmol, 62%) as white solid. ¹H NMR (400 MHz, DMSO) δ 9.66 (s, 2H), 7.13 (d, J=2.6 Hz, 1H), 7.03 (s, 1H), 7.00 (d, J=8.3 Hz, 1H), 6.93 (s, 1H), 6.90 (dd, J=8.3, 2.6 Hz, 1H), 6.87-6.84 (m, 2H), 6.77 (dd, J=8.2, 2.6 Hz, 1H).

### D) Thioether and sulfone “A” group analogues

### Synthesis 5,7-dihydrodibenzo[c,e]thieine-3,9-diol 721

### Step 1: Synthesis 3,9-dimethoxy-5,7-dihydrodibenzo[c,e]thiepine

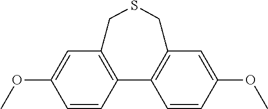

A mixture of 2,2′-bis(bromomethyl)-4,4′-dimethoxy-1,1′-biphenyl (procedure described above) (220 mg, 0.55 mmol) and sodium sulphidehydrate (69 mg, 0.71 mmol) in DMF (3 mL) was heated at 100° C. for 20 min. After cooling, the mixture was poured into water (10 mL), and the precipitate was filtered and washed with water (2×3 mL). The precipitate was taken up in CHCl₃ (15 mL), the solution was dried over Na₂SO₄, and the solvent was evaporated in vacuo to give 3,9-dimethoxy-5,7-dihydrodibenzo[c,e]thiepine (140 mg, 0.510 mmol, 93%) as a yellowish solid. ¹H NMR (400 MHz, CDCl₃) δ 7.19 (d, J=8.4 Hz, 2H), 6.91 (dd, J=8.3, 2.7 Hz, 2H), 6.87 (d, J=2.6 Hz, 2H), 3.86 (s, 6H), 3.56 (d, J=12.7 Hz, 2H), 3.27 (s, 2H).

### Step 2: Synthesis 5,7-dihydrodibenzo[c,e]thiepine-3,9-diol

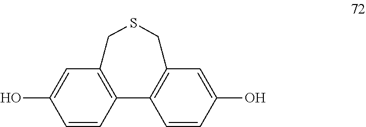 [SMILES: Oc1ccc2c(c1)CSCc1cc(O)ccc1-2]

A solution of BBr₃ (0.59 ml, 0.59 mmol, 1M in DCM) was added at −78° C. to a solution of 3,9-dimethoxy-5,7-dihydrodibenzo[c,e]thiepine (54 mg, 0.20 mmol) in DCM 2 ml dcm and stirring continued overnight at room temperature. Methanol (5 ml) was added at 0° C. and the solvent was removed in vacuo. The crude was purified by MPLC (SiO₂, MeOH/DCM from 0% to 8%) to afford 5,7-dihydrodibenzo[c,e]thiepine-3,9-diol (18 mg, 0.074 mmol, 37%) as a beige solid. R_{f}=0.3 (MeOH/DCM 5%). ¹H NMR (400 MHz, DMSO) δ 9.46 (s, 2H), 7.09-6.91 (m, 2H), 6.78-6.67 (m, 4H), 3.28 (s, 4H).

### Synthesis of 3,9-dihydroxy-5,7-dihydrodibenzo[c,e]thiepine 6,6-dioxide 73

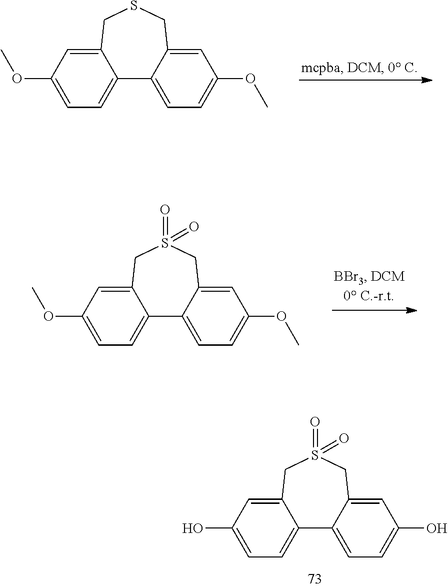

### Step 1: Synthesis of 3,9-dimethoxy-5,7-dihydrodibenzo[c,e]thiepine 6,6-dioxide

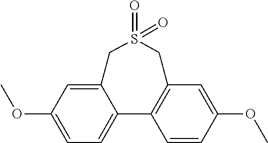

MCPBA (170 mg, 0.690 mmol) was added to 3,9-dimethoxy-5,7-dihydrodibenzo[c,e]thiepine (90 mg, 0.33 mmol) in DCM (2 ml) at 0° c. and the reaction mixture was stirred at room temperature overnight. Na₂S₂O₃ 1M solution was added and the mixture stirred for 10 minutes, then NaHCO₃ saturated solution was added and the mixture was extracted twice with NaHCO₃ saturated solution. The organic phase was dried over sodium sulfate, filtered and evaporated under vacuum. The crude was purified by MPLC (SiO₂, EtOAc/cyclohexane 0% to 30%) to afford 3,9-dimethoxy-5,7-dihydrodibenzo[c,e]thiepine 6,6-dioxide (90 mg, 0.30 mmol, 89%) as a white solid. ¹H NMR (400 MHz, CDCl₃) δ 7.38 (d, J=8.4 Hz, 2H), 7.03 (dd, J=8.4, 2.6 Hz, 2H), 6.99 (d, J=2.6 Hz, 2H), 4.07-3.93 (q, 4H), 3.88 (s, 6H).

### Step 2: Synthesis of 3,9-dihydroxy-5,7 cdihydrodibenzo[c,e]thiepine 6,6-dioxide

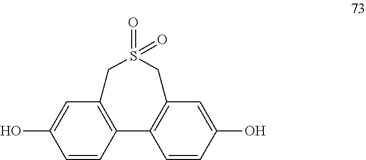 [SMILES: O=S1(=O)Cc2cc(O)ccc2-c2ccc(O)cc2C1]

A solution of BBr₃ (1.0 ml, 1.0 mmol, 1M in DCM, 3.5 eq.) was added at 0° C. to a solution 3,9-dimethoxy-5,7-dihydrodibenzo[c,e]thiepine 6,6-dioxide (90 mg, 0.30 mmol, 1.0 eq.) in DCM (2 mL) and stirring continued overnight at room temperature. Methanol (5 mL) was added at 0° C. and the solvent was removed in vacuo. The crude was purified by MPLC (EtOAc/Hex from 0% to 70%) to afford 1,9-dihydroxy-5,7-dihydrodibenzo[c,e]thiepine 6,6-dioxide (46 mg, 0.17 mmol, 56%) as a beige solid. R_{f}=0.3 (MeOH/DCM 5%). ¹H NMR (400 MHz, DMSO) δ 9.76 (s, 2H), 7.26 (d, J=1H z, 21), 6.94-6.83 (m, 4H), 4, 4.29 (d, J=13.7 Hz, 2H), 3.73 (d, J=13.7 Hz, 2-1)

### E) Amides “A” group analogues

### Synthesis of 3,9-dihydroxy-6,7-dihydro-5H-dibenzo[c,e]azepin-5-one (74)

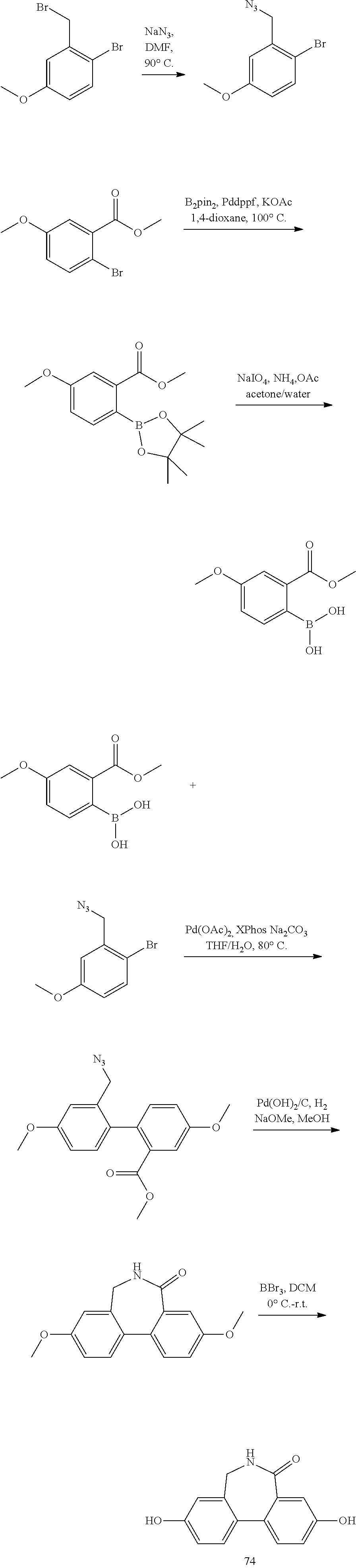

### Step 1: Synthesis of 2-(azidomethyl)-1-bromo-4-methoxybenzene

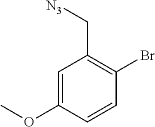

1-bromo-2-(bromomethyl)-4-methoxybenzene (5.00 g, 17.9 mmol, 1.0 eq.) was dissolved in DMF (60 mL) and NaN₃ (5.81 g, 89.3 mmol, 5.0 eq.) were added in one portion. Then the reaction mixture was heated to 90° C. and stirring was continued overnight. After overnight stirring the reaction mixture was allowed to cool to r.t., quenched with water (300 mL) and extracted with cyclohexane (3×75 mL). The combined organic layers were dried over anhydrous Na₂SO₄ and concentrated under vacuo to afford pure 2-(azidomethyl)-1-bromo-4-methoxybenzene (4.32 g, 17.8 mmol, 99%) as a colorless oil. ¹H NMR (400 MHz, CDCl₃) δ 7.47 (d, J=8.8 Hz, 1H), 6.95 (d, J=3.0 Hz, 1H), 6.76 (dd, J=8.8, 3.0 Hz, 1H), 4.45 (s, 2H), 3.81 (s, 3H).

### Step 2: Synthesis of 5-methoxy-2-(4,4,5,5-tetramethyl-1,3,2-dioxaborolan-2-yl)benzoate

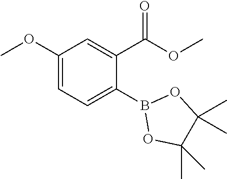

Methyl 2-bromo-5-methoxybenzoate (10.0, 40.8 mmol, 1.0 eq.) was dissolved in 1,4-dioxane (140 mL). To this solution was added B₂pin₂ (11.4 g, 44.9 mmol, 1.1 eq.), Pd(dppf)Cl₂ (1.49 g, 2.04 mmol, 0.1 eq.) and KOAc (12.0 g, 122 mmol, 3.0 eq.) and the reaction mixture was thoroughly degassed for 10 min using a N₂ balloon before putting the reaction mixture in a pre-heated oil-bath to 85° C. for overnight stirring. Upon complete consumption of the starting material, the reaction mixture was allowed to cool to r.t. and then quenched with water. The layers were separated and the aq. phase was extracted with ethyl acetate (2×100 mL). The combined organic layers were dried over anhydrous Na₂SO₄ and concentrated under vacuo. The crude product was purified by MPLC (SiO₂, 240 g, EtOAc in Hex 0-15%) to yield methyl 5-methoxy-2-(4,4,5,5-tetramethyl-1,3,2-dioxaborolan-2-yl)benzoate (9.51 g, 32.6 mmol, 78%) as a pale yellow oil. ¹H NMR (400 MHz, CDCl₃) δ 7.27 (d, J=6.1 Hz, 1H), 7.09 (s, 1H), 6.88 (dd, J=8.1, 2.6 Hz, 1H), 3.73 (s, 3H), 3.67 (s, 3H), 1.23 (s, 12H).

### Step 3: Synthesis of (4-methoxy-2-(methoxycarbonyl)phenyl)boronic acid

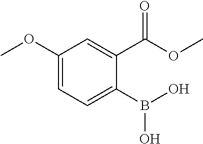

5-methoxy-2-(4,4,5,5-tetramethyl-1,3,2-dioxaborolan-2-yl)benzoate (1.33 g, 4.55 mmol, 1.0 eq.) was dissolved in a mixture of acetone (23 mL) and water (23 mL) and NH₄OAc (1.05 g, 13.7 mmol, 3.0 eq.) as well as NaIO₄ (2.92, 13.7 mmol, 3.0 eq.) were added in one portion. Upon complete addition the mixture warmed up slightly and stirring was continued overnight. After the disappearance of the starting material (as indicated by TLC) the reaction mixture was filtered and the white precipitate was washed with acetone and the mother liquor concentrated to give pure (4-methoxy-2-(methoxycarbonyl)phenyl)boronic acid (590 mg, 2.81 mmol, 62%) as a white solid. Analytical data matched with the literature.

### Step 4: Synthesis of 2′-(azidomethyl)-4,4′-dimethoxy-[1,1′-biphenyl]-2-carboxylate

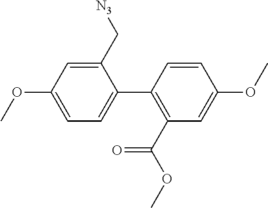

A 20 mL Biotage MW vial was charged with (4-methoxy-2-(methoxycarbonyl)phenyl)boronic acid (563 mg, 2.68 mmol, 1.10 eq.), 2-(azidomethyl)-1-bromo-4-methoxybenzene (590 mg, 2.44 mmol, 1.0 eq.), Pd(OAc)₂ (27 mg, 0.12 mmol, 0.05 eq.), XPhos (116 mg, 0.24 mmol, 0.1 eq.) and all reagents were dissolved in THF (15 mL). The reaction mixture was degassed by using a N₂ balloon for 10 min and afterwards a solution of Na₂CO₃ (775 mg, 7.31 mmol, 3.0 eq.) in water (5 mL) was added dropwise at r.t. Upon complete addition the reaction mixture was heated to 80° C. in an oil-bath and stirring was continued overnight. After overnight stirring the reaction mixture was allowed to cool to r.t. and quenched with water, the layers were separated and the aqueous layer was extracted with ethyl acetate (2×10 mL). The combined organic layers were dried over anhydrous Na₂SO₄ and concentrated under vacuo. The crude product was purified by MPLC (SiO₂, 25 g, EtOAc in Hex 0-20%) to give methyl 2′-(azidomethyl)-4,4′-dimethoxy-[1,1′-biphenyl]-2-carboxylate (367 mg, 1.12 mmol, 46%) as a colorless oil. ¹H NMR (400 MHz, CDCl₃) δ 7.47 (d, J=2.7 Hz, 1H), 7.15 (d, J=8.4 Hz, 1H), 7.08 (dd, J=8.4, 2.8 Hz, 1H), 7.04 (d, J=8.4 Hz, 1H), 6.96 (d, J=2.6 Hz, 1H), 6.87 (dd, J=8.4, 2.7 Hz, 1H), 4.09 (d, J=3.1 Hz, 2H), 3.89 (s, 3H), 3.86 (s, 3H), 3.63 (s, 3H).

### Step 5: Synthesis of 3,9-dimethoxy-6,7-dihydro-5H-dibenzo[c,e]azepin-5-one

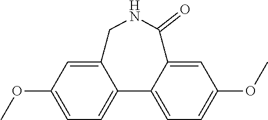

2′-(azidomethyl)-4,4′-dimethoxy-[1,1′-biphenyl]-2-carboxylate (50 mg, 0.15 mmol, 1.0 eq.) was dissolved in MeOH (8 mL) and Pd(OH)₂/C (16 mg, 0.02 mmol, 0.15 eq.) as well as NaOMe (33 mg, 0.15 mmol, 1.0 eq.) were added to the solution which was degassed with N₂ three times followed by a hydrogen atmosphere exchange for three times. The reaction mixture was stirred overnight at r.t. before being filtered over a pad of celite and purified by MPLC (SiO₂, EtOAc in Hex 0-30%) to afford 3,9-dimethoxy-6,7-dihydro-5H-dibenzo[c,e]azepin-5-one (22 mg, 0.08 mmol, 53%) as a white solid. ¹H NMR (400 MHz, DMSO) δ 8.53 (s, 1H), 7.45 (t, J=8.4 Hz, 2H), 7.28 (d, J=2.8 Hz, 1H), 7.11 (dd, J=8.6, 2.7 Hz, 1H), 6.94 (dd, J=12.2, 3.8 Hz, 2H), 3.84 (dd, J=9.5, 3.6 Hz, 1H), 3.82 (s, 3H), 3.78 (s, 3H), 3.18 (d, J=14.8 Hz, 1H).

### Step 6: Synthesis of 3,9-dihydroxy-6,7-dihydro-5H-dibenzo[c,e]azepin-5-one

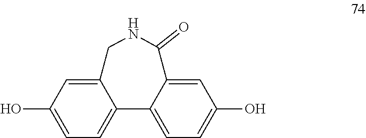 [SMILES: O=C1NCc2cc(O)ccc2-c2ccc(O)cc21]

3,9-dimethoxy-6,7-dihydro-5H-dibenzo[c,e]azepin-5-one (64 mg, 0.24 mmol, 1.0 eq.) was dissolved in DCM (2 mL) and cooled down to 0° C. in an ice-bath and stirring was continued for 5 min. Then BBr₃ (0.95 ml, 1M in DCM, 0.95 mmol, 4.0 eq.) was added dropwise to the reaction mixture. Upon complete addition the mixture was left in the ice bath and was allowed to warm to r.t. over the course of 2 h. When no more starting material could be observed the reaction mixture was dropwise added into 0° C. cold methanol (10 mL) and stirred for an additional 10 min. Then the mixture was concentrated and loaded on silica to be purified by flash column chromatography (SiO₂, 12 g, MeOH in DCM 0-5%) to afford 3,9-dihydroxy-6,7-dihydro-5H-dibenzo[c,e]azepin-5-one (25 mg, 0.10 mmol, 44%) as an orange solid. ¹H NMR (400 MHz, DMSO) δ 8.39 (t, J=6.1 Hz, 1H), 7.33 (d, J=8.4 Hz, 2H), 7.15 (d, J=2.6 Hz, 1H), 6.97 (dd, J=8.6, 2.6 Hz, 1H), 6.80 (dd, J=8.4, 2.3 Hz, 1H), 6.71 (d, J=2.3 Hz, 1H), 3.80 (ddd, J=35.6, 14.6, 6.1 Hz, 2H).

### Synthesis of 3,9-dihydroxy-6-methyl-6,7-dihydro-5H-dibenzo[c,e]azepin-5-one (75)

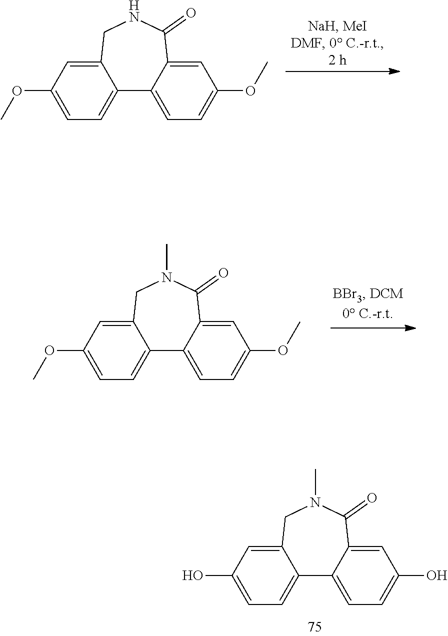

### Step 1: Synthesis of 3,9-dimethoxy-6-methyl-6,7-dihydro-5H-dibenzo[c,e]azepin-5-one

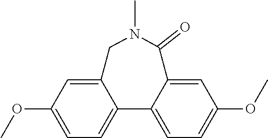

3,9-dimethoxy-6,7-dihydro-5H-dibenzo[c,e]azepin-5-one (80 mg, 0.30 mmol, 1.0 eq.) was dissolved in DMF (3.0 mL) and the solution was cooled to 0° C. in an ice-bath and stirring was continued for 10 min before adding 60% NaH in petroleum oil (14 mg, 0.36 mmol, 1.2 eq.) in one portion. The reaction was stirred until the evolution of hydrogen gas completely ceased upon which Mel (0.13 g, 0.89 mmol, 3.0 eq.) was added dropwise. Afterwards the reaction was allowed to warm up to r.t. and stirred for 3 h until the starting material disappeared (as indicated by TLC). The reaction was quenched with ice-water (10 mL) and the aqueous solution was extracted with diethyl ether (3×10 mL) and the organic layers were washed with water and brine, dried over Na₂SO₄ and concentrated to afford 3,9-dimethoxy-6-methyl-6,7-dihydro-5H-dibenzo[c,e]azepin-5-one (84 mg, 0.30 mmol, 99%) as a white solid. ¹H NMR (400 MHz, CDCl₃) δ 7.35 (dd, J=16.3, 8.4 Hz, 2H), 7.14 (d, J=2.7 Hz, 1H), 6.96 (dd, J=8.5, 2.7 Hz, 1H), 6.90-6.81 (m, 2H), 4.10-3.75 (m, 2H), 3.10 (s, 3H), 3.00 (s, 3H), 2.90 (s, 3H).

### Step 2: Synthesis of 3,9-dihydroxy-6-methyl-6,7-dihydro-5H-dibenzo[c,e]azepin-5-one

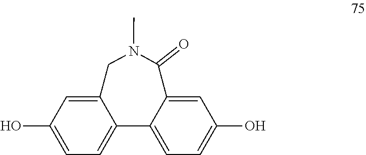 [SMILES: CN1Cc2cc(O)ccc2-c2ccc(O)cc2C1=O]

3,9-dimethoxy-6-methyl-6,7-dihydro-5H-dibenzo[c,e]azepin-5-one (84 mg, 0.84 mmol, 1.0 eq.) was dissolved in DCM (1 mL) and cooled down to 0° C. in an ice-bath and stirring was continued for 5 min. Then BBr₃ (1.20 ml, 1M in DCM, 1.20 mmol, 4.0 eq.) was added dropwise to the reaction mixture. Upon complete addition the mixture was left in the ice bath and was allowed to warm to r.t. over the course of 2 h. When no more starting material could be observed the reaction mixture was dropwise added into 0° C. cold methanol (10 mL) and stirred for an additional 10 min. Then the mixture was concentrated and loaded on silica to be purified by flash column chromatography (SiO₂, 12 g, MeOH in DCM 0-5%) to afford 3,9-dihydroxy-6-methyl-6,7-dihydro-5H-dibenzo[c,e]azepin-5-one (40 mg, 0.16 mmol, 52%) as light orange solid. ¹H NMR (400 MHz, DMSO) δ 9.65 (s, 2H), 7.35 (dd, J=16.3, 8.4 Hz, 2H), 7.14 (d, J=2.7 Hz, 1H), 6.96 (dd, J=8.5, 2.7 Hz, 1H), 6.90-6.81 (m, 2H), 4.20-3.85 (m, 2H), 3.02 (s, 3H).

### Synthesis of 3,9-dihydroxy-5,7-dihydro-6H-dibenzo[b,d]azepin-6-one (76)

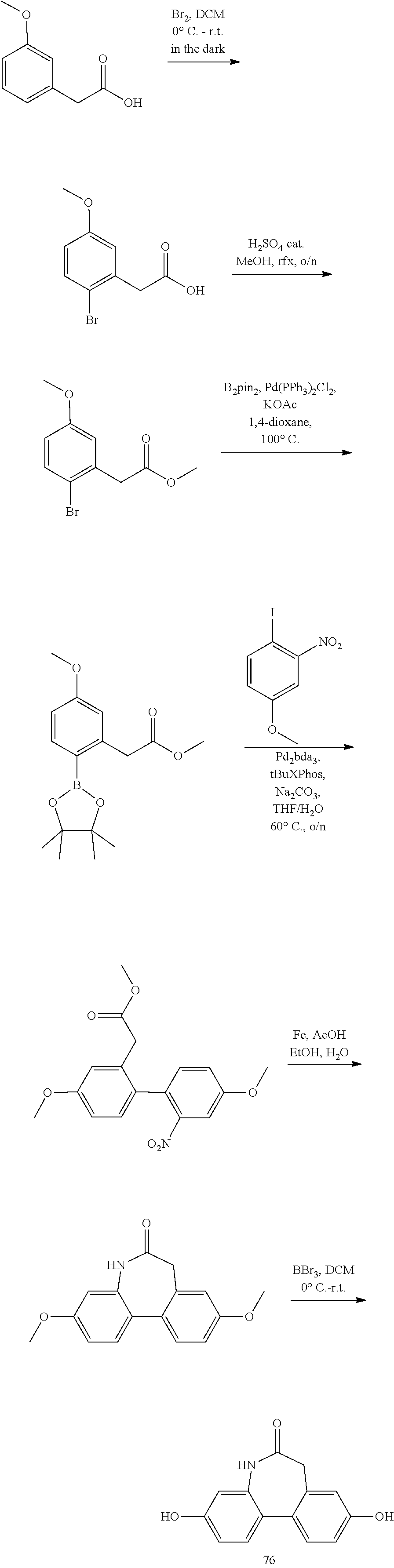

### Step 1: Synthesis of 2-(2-bromo-5-methoxyphenyl)acetic acid

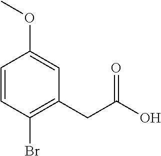

Bromine (1.92 g, 12.0 mmol, 1.0 eq.) was added dropwise to a solution of 2-(3-methoxyphenyl)acetic acid (2.00 g, 12.0 mmol, 1.0 eq.) in DCM (40 mL) at 0° C. Upon complete addition of the bromine, the reaction was allowed to warm to r.t. and stirred overnight while being covered from light using aluminum foil. The dark red solution was discolored with sodium thiosulfate solution (1M), washed with water (50 mL) and separated. The aqueous layer was extracted into DCM (2×25 mL) and the combined organic layers dried over Na₂SO₄, filtered and evaporated to dryness to afford 2-(2-bromo-5-methoxyphenyl)acetic acid (2.80 g, 11.0 mmol, 95%) as a pale red solid.

¹H NMR (400 MHz, CDCl₃) δ 10.07 (s, 1H), 7.45 (d, J=8.8 Hz, 1H), 6.85 (d, J=3.0 Hz, 1H), 6.72 (dd, J=8.8, 3.0 Hz, 1H), 3.79 (s, 2H), 3.78 (s, 3H).

### Step 2: Synthesis of 2-(2-bromo-5-methoxyphenyl)acetate

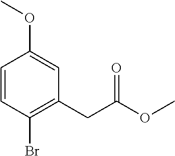

2-(2-bromo-5-methoxyphenyl)acetic acid (6.63 g, 27.1 mmol, 1.0 eq.) was dissolved in MeOH (90 mL) and a catalytic amount of concentrated sulfuric acid (0.2 mL) was added to the mixture which was then refluxed for 4 h before being cooled to r.t., quenched with water and extracted into ethyl acetate (3×100 mL). The organic layers were washed with sat. sodium bicarbonate solution and brine and dried with Na₂SO₄ and concentrated under vacuo. The crude product was purified by MPLC (SiO₂, 240 g, EtOAc in Hex 0-20%) to afford methyl 2-(2-bromo-5-methoxyphenyl)acetate (6.44 g, 24.9 mmol, 92%) as a colorless oil. ¹H NMR (400 MHz, CDCl₃) δ 7.47 (d, J=8.8 Hz, 1H), 6.87 (d, J=3.0 Hz, 1H), 6.74 (dd, J=8.8, 3.0 Hz, 1H), 3.81 (s, 3H), 3.78 (s, 2H), 3.75 (s, 3H).

### Step 3: Synthesis of 2-(5-methoxy-2-(4,4,5,5-tetramethyl-1,3,2-dioxaborolan-2-yl)phenyl)acetate

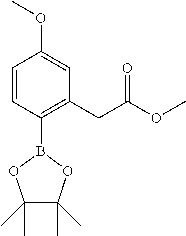

Methyl 2-(2-bromo-5-methoxyphenyl)acetate (2.00 g, 7.72 mmol, 1.0 eq.) was dissolved in 1,4-dioxane (150 mL) and there to was added B₂pin₂ (3.53 g, 13.9 mmol, 1.8 eq.), Pd(PPh₃)₂Cl₂ (542 mg, 0.770 mmol, 0.1 eq.) and KOAc (3.03 g, 30.9 mmol, 4.0 eq.). The resulting reaction mixture was degassed for 10 min using a N₂ balloon before being put into a pre-heated oil-bath at 100° C. overnight. After overnight stirring the mixture was allowed to cool to r.t. and quenched with sat. aq. NH₄Cl solution which was extracted into ethyl acetate (3×75 mL). The combined organic layers were dried over anhydrous Na₂SO₄, concentrated under vacuo and the crude purified by MPLC (SiO₂, 120 g, EtOAc in Hex 0-20%) to afford methyl 2-(5-methoxy-2-(4,4,5,5-tetramethyl-1,3,2-dioxaborolan-2-yl)phenyl)acetate (1.32 g, 4.31 mmol, 56%) as a colorless oil. ¹H NMR (400 MHz, CDCl₃) δ 7.78 (d, J=8.3 Hz, 1H), 6.80 (dd, J=8.3, 2.5 Hz, 1H), 6.74 (d, J=2.5 Hz, 1H), 3.96 (s, 2H), 3.81 (s, 3H), 3.66 (s, 3H), 1.30 (s, 12H).

### Step 4: Synthesis of 2-(4,4′-dimethoxy-2′-nitro-[1,1′-biphenyl]-2-yl)acetate

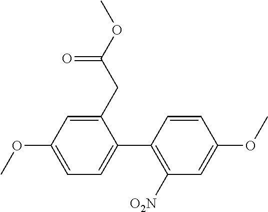

Methyl 2-(5-methoxy-2-(4,4,5,5-tetramethyl-1,3,2-dioxaborolan-2-yl)phenyl)acetate (670 mg, 2.19 mmol, 1.0 eq.) and 1-iodo-4-methoxy-2-nitrobenzene (733 mg, 2.63 mmol, 1.2 eq.) were dissolved in THF (2 mL) and to this solution was added Pd₂dba₃ (100 mg, 0.110 mmol, 0.05 eq.) as well as tBuXPhos (93 mg, 0.22 mmol, 0.1 eq.). The resulting mixture was degassed for 10 min using a N₂ balloon before the dropwise addition of a solution of Na₂CO₃ (696 mg, 6.56 mmol, 3.0 eq.) in water (4 mL). Following the reaction mixture was heated to 60° C. overnight (until starting material completely disappeared on TLC) before being allowed to cool to r.t., quenched with sat. aq. NH₄Cl solution, extracted with ethyl acetate (3×50 mL), dried over anhydrous Na₂SO₄ and concentrated under vacuo. The resulting crude material was purified by MPLC (SiO₂, 40 g, EtOAc in Hex 0-35%) to afford methyl 2-(4,4′-dimethoxy-2′-nitro-[1,1′-biphenyl]-2-yl)acetate (490 mg, 1.48 mmol, 68%) as a green oil. ¹H NMR (400 MHz, CDCl₃) δ 7.48 (d, J=2.7 Hz, 1H), 7.23 (d, J=8.5 Hz, 1H), 7.14 (dd, J=8.5, 2.7 Hz, 1H), 7.03 (d, J=8.4 Hz, 1H), 6.92 (d, J=2.6 Hz, 1H), 6.83 (dd, J=8.4, 2.7 Hz, 1H), 3.91 (s, 3H), 3.84 (s, 3H), 3.59 (s, 3H), 3.48-3.33 (m, 2H).

### Step 5: Synthesis of 3,9-dimethoxy-5,7-dihydro-6H-dibenzo[b,d]azepin-6-one

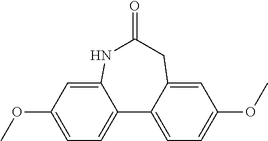

Methyl 2-(4,4′-dimethoxy-2′-nitro-[1,1′-biphenyl]-2-yl)acetate (485 mg, 1.46 mmol, 1.0 eq.) was dissolved in H₂O (3 mL), AcOH (2 mL) and EtOH (3 mL) and powdered Fe (818 mg, 14.6 mmol, 10.0 eq.) was added to the mixture, which was stirred for 2 h until the TLC showed no more starting material. Then reaction mixture was filtered over a pad of celite and concentrated under reduced pressure (AcOH was removed by azeotropic distillation with cyclohexane) and purified by MPLC (SiO₂, 40 g, EtOAc in Hex 0-85%) to afford 3,9-dimethoxy-5,7-dihydro-6H-dibenzo[b,d]azepin-6-one (150 mg, 0.560 mmol, 38%) as a white solid. ¹H NMR (400 MHz, DMSO) δ 9.93 (s, 1H), 7.50 (d, J=8.7 Hz, 1H), 7.44 (d, J=9.0 Hz, 1H), 7.00-6.94 (m, 2H), 6.85 (dd, J=8.7, 2.6 Hz, 1H), 6.72 (d, J=2.6 Hz, 1H), 3.80 (s, 3H), 3.78 (s, 3H).

### Step 6: Synthesis of 3,9-dihydroxy-5,7-dihydro-6H-dibenzo[b,d]azepin-6-one

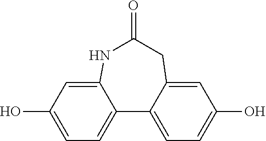 [SMILES: O=C1Cc2cc(O)ccc2-c2ccc(O)cc2N1]

3,9-dimethoxy-5,7-dihydro-6H-dibenzo[b,d]azepin-6-one (70 mg, 0.26 mmol, 1.0 eq.) was dissolved in DCM (2 mL) and cooled down to 0° C. in an ice-bath and stirring was continued for 5 min. Then BBr₃ (1.30 ml, 1M in DCM, 1.30 mmol, 5.0 eq.) was added dropwise to the reaction mixture. Upon complete addition the mixture was left in the ice bath and was allowed to warm to r.t. over the course of 2 h. When no more starting material could be observed the reaction mixture was dropwise added into 0° C. cold methanol (10 mL) and stirred for an additional 10 min. Then the mixture was concentrated and loaded on silica to be purified by flash column chromatography (SiO₂, 12 g, MeOH in DCM 0-5%) to afford 3,9-dihydroxy-5,7-dihydro-6H-dibenzo[b,d]azepin-6-one (35 mg, 0.15 mmol, 56%) as a light yellow solid. ¹H NMR (400 MHz, DMSO) δ 9.82 (s, 1H), 9.56 (s, 2H), 7.33 (d, J=8.5 Hz, 1H), 7.27 (d, J=8.4 Hz, 1H), 6.77 (dd, J=8.4, 2.5 Hz, 1H), 6.69 (d, J=2.5 Hz, 1H), 6.63 (dd, J=8.5, 2.5 Hz, 1H), 6.55 (d, J=2.5 Hz, 1H), 3.20 (s, 2H).

### Synthesis of 3,9-dihydroxy-5-methyl-5,7-dihydro-6H-dibenzo[b,d]azepin-6-one (77)

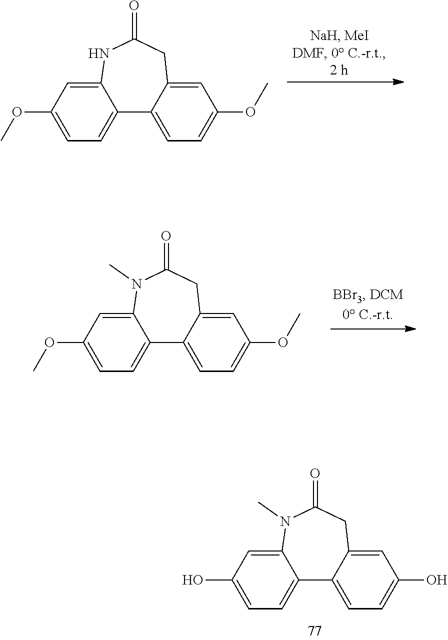

### Step 1: Synthesis of 3,9-dimethoxy-5-methyl-5,7-dihydro-6H-dibenzo[b,d]azepin-6-one

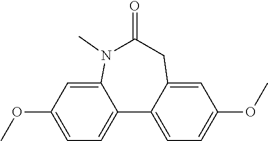

3,9-dimethoxy-5,7-dihydro-6H-dibenzo[b,d]azepin-6-one (85 mg, 0.32 mmol, 1.0 eq.) was dissolved in DMF (3.2 mL) and the solution was cooled to 0° C. in an ice-bath and stirring was continued for 10 min before adding 60% NaH in petroleum oil (14 mg, 0.36 mmol, 1.2 eq.) in one portion. The reaction was stirred until the evolution of hydrogen gas completely ceased upon which Mel (0.060 g, 0.38 mmol, 1.2 eq.) was added dropwise. Afterwards the reaction was allowed to warm up to r.t. and stirred for 3 h until the starting material disappeared (as indicated by TLC). The reaction was quenched with ice-water (10 mL) and the aqueous solution was extracted with diethyl ether (3×10 mL) and the organic layers were washed with water and brine, dried over Na₂SO₄ and concentrated to afford 3,9-dimethoxy-5-methyl-5,7-dihydro-6H-dibenzo[b,d]azepin-6-one (60 mg, 0.21 mmol, 67%) as a light yellow solid. ¹H NMR (400 MHz, CDCl₃) δ 7.46 (dd, J=8.4, 5.6 Hz, 2H), 6.98-6.85 (m, 4H), 3.89 (s, 3H), 3.86 (s, 3H), 3.56-3.39 (dd, 2H), 3.33 (s, 3H).

### Step 1: Synthesis of 3,9-dihydroxy-5-methyl-5,7-dihydro-6H-dibenzo[b,d]azepin-6-one

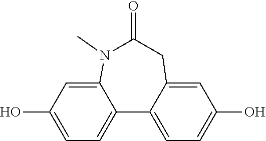 [SMILES: CN1C(=O)Cc2cc(O)ccc2-c2ccc(O)cc21]

3,9-dimethoxy-5,7-dihydro-6H-dibenzo[b,d]azepin-6-one (58 mg, 0.20 mmol, 1.0 eq.) was dissolved in DCM (2 mL) and cooled down to 0° C. in an ice-bath and stirring was continued for 5 min. Then BBr₃ (0.82 ml, 1M in DCM, 0.82 mmol, 4.0 eq.) was added dropwise to the reaction mixture. Upon complete addition the mixture was left in the ice bath and was allowed to warm to r.t. over the course of 2 h. When no more starting material could be observed the reaction mixture was dropwise added into 0° C. cold methanol (10 mL) and stirred for an additional 10 min. Then the mixture was concentrated and loaded on silica to be purified by flash column chromatography (SiO₂, 12 g, MeOH in DCM 0-5%) to afford 3,9-dihydroxy-5-methyl-5,7-dihydro-6H-dibenzo[b,d]azepin-6-one (30 mg, 0.12 mmol, 57%) as light orange solid. ¹H NMR (400 MHz, DMSO) δ 9.74 (s, 1H), 9.57 (s, 1H), 7.31 (dd, J=8.4, 2.3 Hz, 2H), 6.82-6.71 (m, 4H), 3.31-3.20 (m, 2H), 3.15 (s, 3H).

Compound 77A was prepared by employing the appropriate methyl substituted iodophenyl intermediate in the Pd coupling step of the above synthesis of 76, which provides the methyl substituted analog of 76. The remaining steps are analogous to those employed to provide compound 77, i.e. amide methylation followed by deprotection.

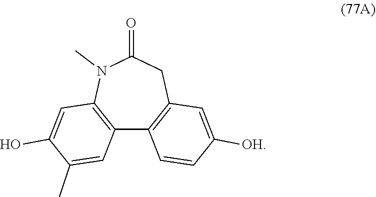 [SMILES: Cc1cc2c(cc1O)N(C)C(=O)Cc1cc(O)ccc1-2]

### Example 2: Synthesis of additional representative compounds

Reactions were not carried out under an inert atmosphere unless specified, and all solvents and commercial reagents were used as received.

Purification by chromatography refers to purification using the COMBIFLASH® Companion purification system or the Biotage SP1 purification system. Where products were purified using an Isolute® SPE Si II cartridge, ‘Isolute SPE Si cartridge’ refers to a pre-packed polypropylene column containing unbonded activated silica with irregular particles with average size of 50 μm and nominal 60 Å porosity. Fractions containing the required product (identified by TLC and/or LCMS analysis) were pooled, the organic fraction recovered by evaporation, to give the final product. Where thin layer chromatography (TLC) has been used, it refers to silica-gel TLC plates, typically 3×6 cm silica-gel on aluminium foil plates with a fluorescent indicator (254 nm), (e.g. Fluka 60778). Microwave experiments were carried out using a Biotage Initiator 60™ which uses a single-mode resonator and dynamic field tuning. Temperatures from 40-250° C. can be achieved, and pressures of up to 30 bar can be reached.

NMR spectra were obtained on a Bruker Avance 400 MHz, 5 mm QNP probe H, C, F, P, single Z gradient, two channel instrument running TopSpin 2.1 or on a Bruker Avance III 400 MHz, 5 mm BBFO Plus probe, single Z gradient, two channel instrument running TopSpin 3.0.

Analytical LC-MS Conditions

Method 1: Experiments were performed on a Waters Acquity SQD2 mass spectrometer linked to a Waters Acquity UPLC binary pump/PDA detector. The spectrometer had an electrospray source operating in positive and negative ion mode. Additional detection was achieved using an Acquity UPLC HSS C18 1.7 μm, 100×2.1 mm column maintained at 40° C. and a 0.4 mL/minute flow rate. The initial solvent system was 95% water containing 0.1% formic acid (solvent A) and 5% MeCN containing 0.1% formic acid (solvent B) for the first 0.4 minute followed by a gradient up to 5% solvent A and 95%.

Method 2: Experiments were performed on a Waters Acquity SQD2 mass spectrometer linked to a Waters Acquity UPLC binary pump/PDA detector. The spectrometer had an electrospray source operating in positive and negative ion mode. Additional detection was achieved using an Acquity UPLC BEH Shield RP18 1.7 μm 100×2.1 mm. column maintained at 40° C. and a 0.4 mL/minute flow rate. The initial solvent system was 95% water containing 0.03% aqueous ammonia (solvent A) and 5% MeCN containing 0.03% aqueous ammonia (solvent B) for the first 0.4 minute followed by a gradient up to 5% solvent A and 95% solvent B over the next 5.4 min. The final solvent system was held constant for a further 0.8 min.

Method 3: Experiments were performed on a Waters Acquity ZQ mass spectrometer linked to a Waters Acquity UPLC binary pump/PDA detector. The spectrometer had an electrospray source operating in positive and negative ion mode. Additional detection was achieved using an Acquity UPLC BEH C18 1.7 μm, 100×2.1 mm column maintained at 40° C. and a 0.4 mL/minute flow rate. The initial solvent system was 95% water containing 0.1% formic acid (solvent A) and 5% MeCN containing 0.1% formic acid (solvent B) for the first 0.4 minute followed by a gradient up to 5% solvent A and 95% solvent B over the next 5.6 min. The final solvent system was held constant for a further 0.8 min.

Method 4: Experiments were performed on a Waters Acquity ZQ mass spectrometer linked to a Waters Acquity UPLC binary pump/PDA detector. The spectrometer had an electrospray source operating in positive and negative ion mode. Additional detection was achieved using an Acquity UPLC BEH C8 1.7 μm, 100×2.1 mm column maintained at 40° C. and a 0.4 mL/minute flow rate. The initial solvent system was 95% water containing 0.03% aqueous ammonia (solvent A) and 5% MeCN containing 0.03% aqueous ammonia (solvent B) for the first 0.4 minute followed by a gradient up to 5% solvent A and 95% solvent B over the next 4 min. The final solvent system was held constant for a further 0.8 min.

Method 5: Experiments were performed on a Waters Acquity ZQ mass spectrometer linked to a Waters Acquity H-class UPLC with DAD detector and QDa. The spectrometer had an electrospray source operating in positive and negative ion mode. Additional detection was achieved using an Acquity UPLC CSH 1.7 μm, 50×2.1 mm column maintained at 40° C. and a 1.0 mL/minute flow rate. The initial solvent system was 97% water containing 0.1% formic acid (solvent A) and 3% MeCN containing 0.1% formic acid (solvent B) for the first 0.4 minute followed by a gradient up to 1% solvent A and 99% solvent B over the next 1.4 min. The final solvent system was held constant for a further 0.5 min.

Method 6: Experiments were performed on a Waters Acquity ZQ mass spectrometer linked to a Waters Acquity H-class UPLC with DAD detector and QDa. The spectrometer had an electrospray source operating in positive and negative ion mode. Additional detection was achieved using an Acquity BEH UPLC 1.7 μm, 50×2.1 mm column maintained at 40° C. and a 0.8 mL/minute flow rate. The initial solvent system was 97% of 7.66 mM ammonia in water (solvent A) and 3% of 7.66 mM ammonia in MeCN containing (solvent B) for the first 0.4 minutes followed by a gradient up to 3% solvent A and 97% solvent B over the next 1.6 min. The final solvent system was held constant for a further 0.5 min.

### A) Ester “A” group analogues

### General Procedure B

### N-(8-Methoxy-6-oxo-6H-benzo[c]chromen-3-yl)methanesulfonamide (79)

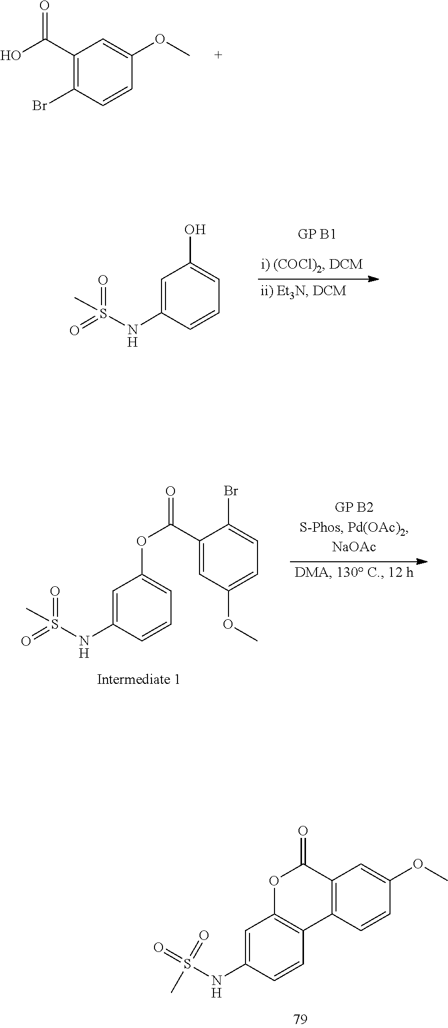 [SMILES: COc1ccc(Br)c(C(=O)O)c1 ; CS(=O)(=O)Nc1cccc(O)c1 ; COc1ccc(Br)c(C(=O)Oc2cccc(NS(C)(=O)=O)c2)c1 ; COc1ccc2c(c1)c(=O)oc1cc(NS(C)(=O)=O)ccc12]

### GP B1

### 3-(Methylsulfonamido)phenyl 2-bromo-5-methoxybenzoate (Intermediate 1)

To a suspension of 2-bromo-5-methoxybenzoic acid (642 mg, 2.78 mmol) in DCM (10 mL) was added oxalyl chloride (0.27 mL, 3.06 mmol) dropwise and 1 drop of DMF. The solution was stirred at RT for 1 h and the solvent was removed in vacuo. The resultant mixture was re-dissolved in DCM (5 mL) and a suspension of N-(3-hydroxyphenyl)methanesulfonamide (520 mg, 2.78 mmol) in DCM (5 mL) was added followed by TEA (0.58 mL, 4.17 mmol). The resulting mixture was stirred for 4 h, then diluted with DCM and washed with saturated aq. NH₄Cl. The organic extracts were filtered through PTFE and concentrated in vacuo and the crude product was purified by chromatography on silica (ISCO 12 g) using 0-50% EtOAc in cyclohexane as eluant to give the product 3-(methylsulfonamido)phenyl 2-bromo-5-methoxybenzoate as colourless oil (1 g, 90%). LCMS (Method 5): Rₜ 1.43 min; m/z 398.0/400.0 [M−H]⁻. ¹H NMR (400 MHz, CDCl₃) δ 7.61 (1H, d, J=8.9 Hz), 7.52 (1H, d, J=3.1 Hz), 7.41 (1H, t, J=8.1 Hz), 7.19-7.08 (3H, m), 6.98 (1H, dd, J=8.9, 3.1 Hz), 6.77 (1H, s), 3.87 (3H, s), 3.07 (6H, s).

### GP B2

### N-(8-Methoxy-6-oxo-6H-benzo[c]chromen-3-yl)methanesulfonamide (79)

A mixture of 3-(methylsulfonamido)phenyl 2-bromo-5-methoxybenzoate (Intermediate 1) (900 mg, 2.26 mmol), SPhos (92 mg, 0.225 mmol), palladium(II)acetate (50 mg, 0.225 mmol) and sodium acetate (369 mg, 4.5 mmol) in DMA (45 mL) was placed in a sealed tube, degassed and purged with argon (×3). The mixture was heated to 130° C. for 3 h then cooled and diluted with water (400 mL) and extracted into DCM (3×50 mL) and the combined organic extracts were washed with brine and evaporated in vacuo at 80° C. to remove residual DMA. The crude mixture was recrystallised from MeCN to give the product N-(8-methoxy-6-oxo-6H-benzo[c]chromen-3-yl)methanesulfonamide as a cream solid (200 mg, 27%). LCMS (Method 3): Rₜ=3.85 min; m/z=320.0 [M+H]⁺. ¹H NMR (400 MHz: DMSO-d₆) δ 10.22 (1H, s), 8.30 (1H, d, J=8.6 Hz), 8.25 (1H, d, J=9.2 Hz), 7.65 (1H, d, J=2.8 Hz), 7.54 (1H, dd, J=8.9, 2.8 Hz), 7.22-7.19 (2H, m), 3.92 (3H, s), 3.11 (3H, s).

### 3-Chloro-8-hydroxy-6H-benzo[c]chromen-6-one (80)

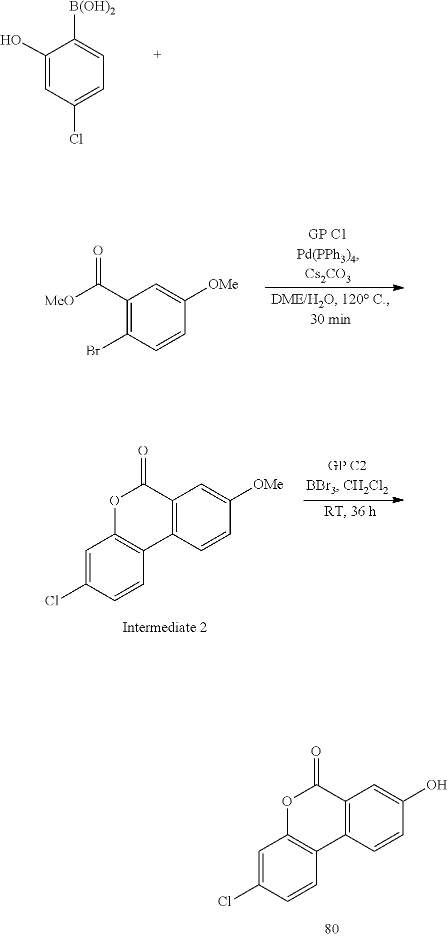 [SMILES: OB(O)c1ccc(Cl)cc1O ; COC(=O)c1cc(OC)ccc1Br ; COc1ccc2c(c1)c(=O)oc1cc(Cl)ccc12 ; O=c1oc2cc(Cl)ccc2c2ccc(O)cc12]

### GP C1

### 3-Chloro-8-methoxy-6H-benzo[c]chromen-6-one (Intermediate 2)

To a solution of 4-chloro-2-hydroxyphenylboronic acid (253 mg, 1.47 mmol) in DME (8.0 mL) and water (2.0 mL) was added methyl 2-bromo-5-methoxybenzoate (300 mg) and cesium carbonate (1.60 g, 4.90 mmol) followed by tetrakis(triphenylphosphine)palladium(0) (141 mg, 0.122 mmol). The reaction mixture was heated at 120° C. in a microwave for 30 mins. The mixture was diluted with EtOAc (100 mL) and washed with water (10 mL) and brine (10 mL). The organic layer was passed through a phase separator and concentrated in vacuo. Purification of the residue by chromatography on silica eluting with 5-15% EtOAc in cyclohexane followed by trituration in MeOH and drying in a vacuum oven afforded the title compound as a white solid (112 mg, 35%). LCMS (Method 1). Rₜ=5.51 min; m/z=261.0, 263.1 [M+H]⁺. ¹H NMR (400 MHz: CDCl₃) δ 7.99 (1H, d, J=8.8 Hz), 7.91 (1H, d, J=8.3 Hz), 7.81 (1H, d, J=2.8 Hz), 7.44-7.36 (2H, m), 7.31 (1H, dd, J=8.6, 2.0 Hz), 3.95 (3H, s);

### GP C2

### 3-Chloro-8-hydroxy-6H-benzo[c]chromen-6-one (80)

To a solution of 3-chloro-8-methoxy-6H-benzo[c]chromen-6-one (Intermediate 2) (70 mg, 0.268 mmol) in dry DCM (10 mL) under nitrogen was added dropwise a solution of boron tribromide in DCM (1.0 M, 5.4 mL, 5.36 mmol). The reaction mixture was stirred at RT for 3 days. Water (20 mL) was added and the mixture was diluted with DCM (10 mL). The mixture was stirred at RT for 10 mins. The resulting precipitate was filtered off and the aqueous layer was extracted with DCM (2×50 mL). The combined organic layers were passed through a phase separation cartridge and concentrated under reduced pressure. The precipitate was dissolved in MeOH/DCM and concentrated in vacuo. Purification of the combined residues by chromatography on silica eluting with 2-4% MeOH in DCM afforded the title compound as a white solid (28 mg, 42%). LCMS (Method 1): Rₜ=4.55 min; m/z=247.1, 249.0 [M+H]⁺. ¹H NMR (400 MHz: DMSO-d6) δ 10.54 (1H, s), 8.35-8.31 (2H, m), 7.63-7.61 (2H, m), 7.51-7.42 (2H, m).

### General Procedure D

### 2-(Dimethylamino)-N-(6-oxo-6H-benzo[c]chromen-3-yl)acetamide (81)

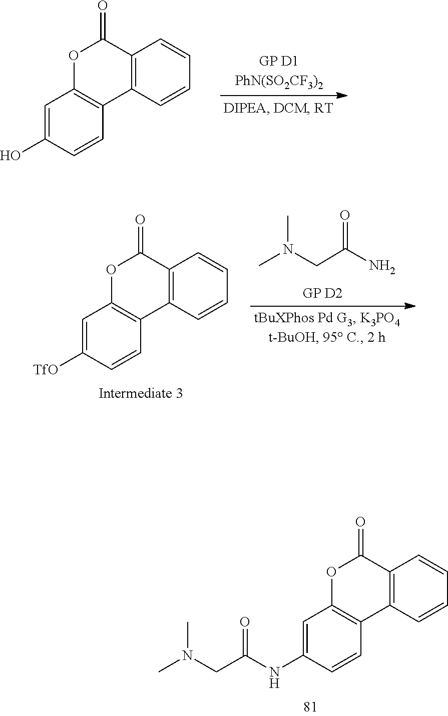

### GP D1

### 6-Oxo-6H-benzo[c]chromen-3-yl trifluoromethanesulfonate (Intermediate 3)

A mixture of 6-hydroxy-6H-benzo[c]chromen-6-one (2.50 g, 11.78 mmol), N-phenyl-bis(trifluoromethanesulfonimide) (5.05 g, 14.1 mmol) and DIPEA (4.1 mL, 23.6 mmol) in DCM (50 mL) was stirred under nitrogen at RT. A catalytic amount of DMAP was added and the mixture stirred for 48 h. The resulting red solution was washed with 1M HCl (50 mL) and the DCM layer was dried (PTFE frit) and evaporated. The crude residue was recrystallised from DCM/cyclohexane to give the product as a cream solid. The mother liquors were purified by chromatography on silica using 20-100% DCM in cyclohexane as eluant. This afforded additional product 1.22 g (total yield 2.86 g, 71%). LCMS (Method 5): Rₜ=1.60 min (no m/z detected—poor ionization). ¹H NMR (CDCl₃) δ 8.43 (1H, dd, J=1.3, 8.0 Hz), 8.16 (1H, d, J=8.9 Hz), 8.11 (1H, d, J=8.0 Hz), 7.92-7.87 (1H, m), 7.69-7.64 (1H, m), 7.34 (1H, d, J=2.3 Hz), 7.30 (1H, dd, J=2.5, 8.9 Hz).

### GP D2

### 2-(Dimethylamino)-N-(6-oxo-6H-benzo[c]chromen-3-yl)acetamide (81)

A mixture of 6-oxo-6H-benzo[c]chromen-3-yl trifluoromethanesulfonate (Intermediate 3) (344 mg, 1.0 mmol), 2-(dimethylamino)acetamide (153 mg, 1.5 mmol), tBuXPhos-Pd-G3 (24 mg, 0.03 mmol) and potassium phosphate tribasic (318 mg, 1.5 mmol) in a septum-sealed vial was degassed (evacuation and flush with argon 3 cycles). Warm degassed (argon sparged) tert-butanol (8.5 mL) was added via syringe and the mixture was then heated at 95° C. for 2 h. The cooled mixture was diluted with water (15 mL) and the resulting mixture was filtered and dried in vacuo to afford a grey solid. This was taken into DCM (15 mL) and filtered through a 2 g flash Si (II) cartridge which was then further eluted with 2% MeOH in DCM to give the title compound (125 mg, 42%) a white solid. LCMS (Method 3): Rₜ=2.72 min; m/z=296.9 [M+H]+.

¹H NMR (400 MHz, DMSO-d₆) δ 10.14 (1H, s), 8.37 (1H, d, J=8.1 Hz), 8.29 (1H, d, J=8.8 Hz), 8.23 (1H, dd, J=1.1, 7.9 Hz), 7.96-7.90 (1H, m), 7.88 (1H, d, J=2.1 Hz), 7.68 (1H, dd, J=2.1, 8.7 Hz), 7.66-7.60 (1H, m), 3.13 (2H, s), 2.30 (6H, s).

### General Procedure E

### Methyl (6-oxo-6H-benzo[c]chromen-3-yl)carbamate (82)

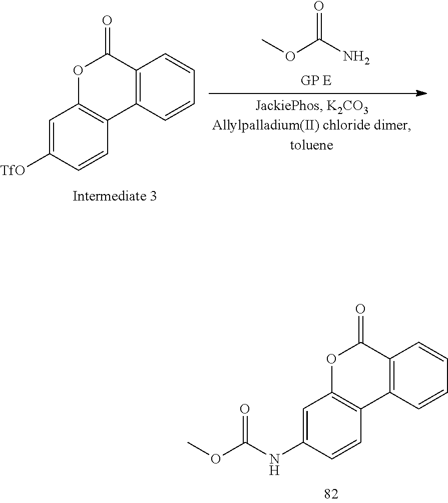

A mixture of 6-oxo-6H-benzo[c]chromen-3-yl trifluoromethanesulfonate (Intermediate 3) (250 mg, 0.73 mmol), methyl carbamate (82 mg, 1.09 mmol), allylpalladium(II)chloride dimer (2.7 mg, 0.007 mmol), JackiePhos (29 mg, 0.036 mmol) and K₂CO₃ (301 mg, 2.18 mmol) in toluene (6.0 mL) was sparged with argon for 5 min. The reaction vessel was then sealed, and the mixture was heated at 110° C. for 1 h. The cooled reaction mixture was diluted with DCM (20 mL) and water (20 mL), which gave a suspension in the aqueous phase. The organic phase was separated, and the aqueous phase was washed with DCM (20 mL). The aqueous phase was filtered and the dark solid recovered was taken into 6% MeOH in DCM. This solution was filtered through a 5 g flash Si (II) cartridge which was then further eluted with 6% MeOH in DCM to afford the title compound (129 mg, 65%) as a white solid. LCMS (Method 3): Rₜ=4.05 min; m/z=269.9 [M+H]⁺. ¹H NMR (400 MHz, DMSO-d₆) δ 10.12 (1H, s), 8.32 (1H, d, J=8.1 Hz), 8.28 (1H, d, J=8.8 Hz), 8.22 (1H, dd, J=1.1, 7.9 Hz), 7.95-7.89 (1H, m), 7.65-7.58 (2H, m), 7.45 (1H, dd, J=2.1, 8.7 Hz), 3.72 (3H, s).

### General Procedure F

### 3-Bromo-8-methoxy-6H-benzo[c]chromen-6-one (84)

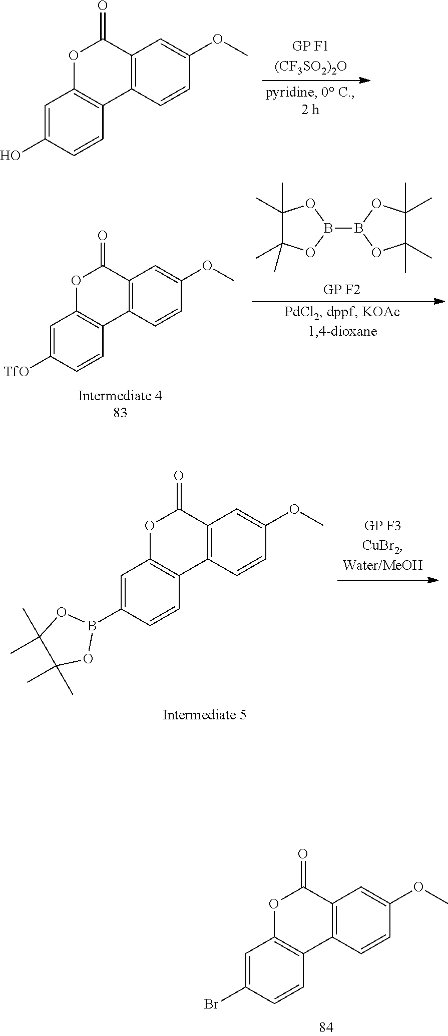

### GP F1

### 8-Methoxy-6-oxo-6H-benzo[c]chromen-3-yl trifluoromethanesulfonate (83) (Intermediate 4)

3-Hydroxy-8-methoxy-6H-benzo[c]chromen-6-one (1 g, 4.13 mmol) was dissolved in pyridine (10 mL) and the mixture was cooled in ice-water. Trifluoromethanesulfonic anhydride (1 mL, 6.19 mmol) was added dropwise and the resulting brown mixture was stirred at 0° C. to RT for 2 h. The mixture was concentrated in vacuo and the residue was dissolved in DCM and washed with 1M HCl, brine, dried (PTFE frit) and concentrated in vacuo. The resultant residue was passed through a silica pad (12 g) and the product was eluted with 50-100% DCM in cyclohexane to give the compound as white crystals (1.2 g, 80%). ¹H NMR (400 MHz, CDCl₃) δ 8.56 (1H, d, J=9.1 Hz), 8.48 (1H, d, J=8.8 Hz), 7.80 (1H, d, J=2.5 Hz), 7.75 (1H, d, J=2.8 Hz), 7.65 (1H, dd, J=2.8, 8.8 Hz), 7.59 (1H, dd, J=2.7, 9.0 Hz), 4.00 (3H, s); LCMS (Method 1): Rₜ=5.64 min; m/z=375.0 [M+H]⁺.

### GP F2

### 8-Methoxy-3-(4,4,5,5-tetramethyl-1,3,2-dioxaborolan-2-yl)-6H-benzo[c] chromen-6-one (Intermediate 5)

A mixture of 8-methoxy-6-oxo-6H-benzo[c]chromen-3-yl trifluoromethanesulfonate (Intermediate 4) (1.0 g, 2.67 mmol), potassium acetate (393 mg, 4.0 mmol), [1,1-bis(diphenylphosphino)ferrocene] dichloropalladium(II) complex with DCM (65 mg, 0.08 mmol), 1,1-bis(diphenylphosphino)ferrocene (44 mg, 0.08 mmol) and dioxane (20 mL) was sparged with argon. Bis(pinacolato)diboron (746 mg, 2.94 mmol) was added and after a further period of degassing, the mixture was heated at 90° C. under argon for 19 h. The cooled mixture was partitioned between ether (25 mL) and water (25 mL) and the phases were separated, and the aqueous phase extracted with ether (2×25 mL). The combined organic extract was washed with saturated brine, dried (Na₂SO₄) and concentrated in vacuo. The residue was purified by flash chromatography on a 20 g Si-(II) cartridge eluting with DCM then 10% EtOAc in DCM. The product obtained was triturated with cyclohexane (10 mL) then dried in vacuo to afford the title compound (0.76 g, 81%) as an off white solid. LCMS (Method 5): Rₜ=1.65 min; m/z=353.1 [M+H]⁺ and Rₜ=1.11 min; m/z=271.1 [M−Pin+H]⁺.

### GP F3

### 3-Bromo-8-methoxy-6H-benzo[c]chromen-6-one (84)

A suspension of 8-methoxy-3-(4,4,5,5-tetramethyl-1,3,2-dioxaborolan-2-yl)-6H-benzo[c]chromen-6-one (Intermediate 5) (352 mg, 1.0 mmol) in MeOH (10 mL) was treated with a solution of copper(II)bromide (670 mg, 3.0 mmol) in water (10 mL). The resulting mixture was heated at reflux for 16 h then cooled. The cold mixture was extracted with ether (2×25 mL) then DCM (2×25 mL) and the combined organic phase was filtered through a hydrophobic frit then concentrated in vacuo. The residue was purified by flash chromatography on a 5 g Si-(II) cartridge and eluted with [1:1] DCM/cyclohexane then DCM to afford the title compound (240 mg, 78%) as a white solid. 1H NMR (400 MHz, CDCl₃) δ 8.00 (1H, d, J=8.9 Hz), 7.84 (1H, d, J=8.7 Hz), 7.80 (1H, d, J=3.0 Hz), 7.53 (1H, d, J=1.9 Hz), 7.46-7.39 (2H, m), 3.95 (3H, s); LCMS (Method 5), Rₜ=1.53 min; m/z=304.8, 306.8 [M+H]⁺.

### Procedure G

### 8-(Difluoromethyl)-3-(methoxymethoxy)-6H-benzo[c]chromen-6-one (85)

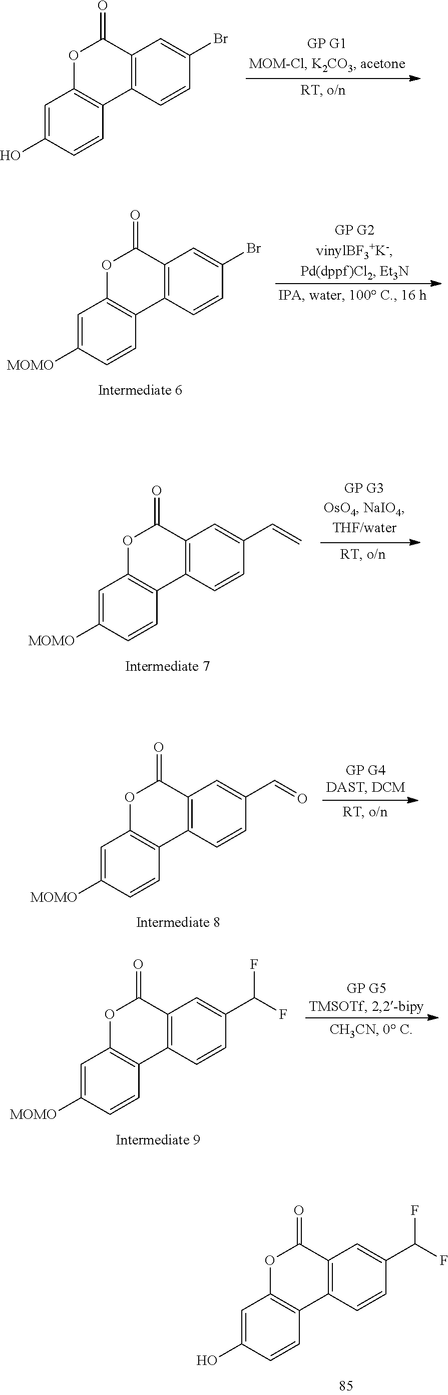

### GP G1

### 8-Bromo-3-(methoxymethoxy)-6H-benzo[c]chromen-6-one (Intermediate 6)

8-Bromo-3-hydroxy-6H-benzo[c]chromen-6-one (1.0 g, 3.44 mmol), K₂CO₃ (1.42 g, 10.31 mmol) and chloromethyl methyl ether (0.39 mL, 5.15 mmol) were suspended in acetone (10 mL) and the mixture was stirred for 3 h. An additional aliquot of chloromethyl methyl ether (0.39 mL, 5.15 mmol) was added and the mixture stirred for 2 h. The mixture was concentrated in vacuo and dispersed between DCM and water. The DCM layer was washed with brine, dried (PTFE frit) and evaporated to give the product as a white solid (1 g, 86%). ¹H NMR (400 MHz, CDCl₃) δ 8.50 (1H, s), 7.9-7.88 (3H, m), 7.07-7.02 (2H, m), 5.25-5.24 (2H, m), 3.51 (3H, s).

### GP G2

### 3-(Methoxymethoxy)-8-vinyl-6H-benzo[c]chromen-6-one (Intermediate 7)

A mixture of 8-bromo-3-(methoxymethoxy)-6H-benzo[c]chromen-6-one (Intermediate 6) (1 g, 2.98 mmol), potassium vinyltrifluoroborate (520 mg, 3.88 mmol), TEA (1.2 mL, 8.95 mmol) and [1,1′-bis(diphenylphosphino)ferrocene]dichloropalladium(II) complex with DCM (122 mg, 0.15 mmol) in isopropanol (20 mL) and water (10 mL) was placed in a sealed tube, evacuated and purged with argon (×3). The mixture was heated at 90° C. under argon for 2 h. The cooled mixture was concentrated in vacuo and the residue dispersed between EtOAc and water. The EtOAc layer was washed with brine, dried (PTFE frit) and concentrated in vacuo. The resultant residue was purified by chromatography on silica using 0-50% DCM in cyclohexane as eluant to give the product as a white solid (705 mg, 71%). ¹H NMR (400 MHz, CDCl₃) δ 8.37 (1H, d, J=1.9 Hz), 7.98 (1H, d, J=8.5 Hz), 7.94 (1H, d, J=8.7 Hz), 7.84 (1H, dd, J=2.0, 8.4 Hz), 7.07-7.02 (2H, m), 6.81 (1H, dd, J=10.9, 17.6 Hz), 5.91 (1H, d, J=17.6 Hz), 5.40 (1H, d, J=11.0 Hz), 5.25 (2H, s), 3.51 (3H, s).

### GP G3

### 3-(Methoxymethoxy)-6-oxo-6H-benzo[c]chromene-8-carbaldehyde (Intermediate 8)

To a solution of 3-(methoxymethoxy)-8-vinyl-6H-benzo[c]chromen-6-one (Intermediate 7) (700 mg, 2.48 mmol) in THF (40 mL) was added osmium tetroxide (0.25 mL, 0.025 mmol) followed by a solution of sodium periodate (1.59 g, 7.44 mmol), and the resulting solution was stirred for 18 h, which gave a white suspension. The mixture was concentrated in vacuo and the residue portioned between DCM and water, and the DCM layer was washed with aqueous sodium sulfite, brine then dried (PTFE frit) to give a white solid (700 mg, quant.). ¹H NMR (400 MHz, CDCl₃) δ 10.11 (1H, s), 8.83 (1H, d, J=1.8 Hz), 8.30 (1H, dd, J=1.8, 8.4 Hz), 8.16 (1H, d, J=8.4 Hz), 8.04-8.00 (1H, m), 7.10-7.07 (2H, m), 5.27 (2H, s), 3.52 (3H, s); LCMS (Method 6): Rₜ=1.41 min; m/z=284.2 [M+1]⁺.

### GP G4

### 8-(Difluoromethyl)-3-(methoxymethoxy)-6H-benzo[c]chromen-6-one (Intermediate 9)

A suspension of 3-(methoxymethoxy)-6-oxo-6H-benzo[c]chromene-8-carbaldehyde (Intermediate 8) (190 mg, 0.67 mmol) in DCM (3 mL) was placed under argon. DAST (0.26 mL, 2.01 mmol) was added dropwise and the resulting mixture was stirred for 18 h at RT. The resulting solution was neutralised with saturated aqueous NaHCO₃ and the DCM layer was washed with brine, dried (PTFE frit) and concentrated in vacuo. The residue was purified by chromatography on silica using 0-70% DCM in cyclohexane as eluant to give the product as a pale-yellow solid (175 mg, 85%). ¹H NMR (400 MHz, CDCl₃, 258114) δ 8.49 (1H, d, J=1.1 Hz), 8.12 (1H, d, J=8.4 Hz), 7.98 (1H, d, J=8.5 Hz), 7.96-7.92 (1H, m), 7.09-7.05 (2H, m), 6.76 (1H, t, J=56.1 Hz), 5.26 (2H, s), 3.51 (3H, s); LCMS (Method 6): Rₜ=1.55 min (no m/z detected—poor ionization).

### GP G5

### 8-(Difluoromethyl)-3-hydroxy-6H-benzo[c]chromen-6-one (85)

A solution of 8-(difluoromethyl)-3-(methoxymethoxy)-6H-benzo[c]chromen-6-one (Intermediate 9) (65 mg, 0.21 mmol) and 2,2′-bipyridyl in MeCN was placed in a sealed tube under argon and cooled in ice-water. Trifluoromethyl trifluoromethane sulfonate (0.08 mL, 0.42 mmol) was added and the solution was stirred for 18 h. The resultant mixture was stirred with water (0.5 mL) for 30 min. then concentrated in vacuo, and the residue was partitioned between EtOAc and water. The EtOAc layer was washed with brine, dried (PTFE frit) and concentrated in vacuo. The crude residue was purified by chromatography on silica using 0-5% MeOH in DCM as eluant to give the product as a pale-yellow solid. The product was further purified by chromatography on silica using 0-50% EtOAc in cyclohexane as eluant to give the title compound as a white solid (25 mg, 45% yield). ¹H NMR (400 MHz, DMSO-d₆) δ 10.50 (1H, s), 8.41 (1H, d, J=8.5 Hz), 8.35 (1H, d, J=1.1 Hz), 8.21 (1H, d, J=8.9 Hz), 8.04 (1H, d, J=8.4 Hz), 7.21 (1H, t, J=55.6 Hz), 6.88 (1H, dd, J=2.4, 8.7 Hz), 6.78 (1H, d, J=2.4 Hz). LCMS (Method 3): Rₜ=4.03 min; m/z=260.9 [M−H]⁻.

### Procedure H

### 3-Amino-8-methoxy-6H-benzo[c]chromen-6-one (86)

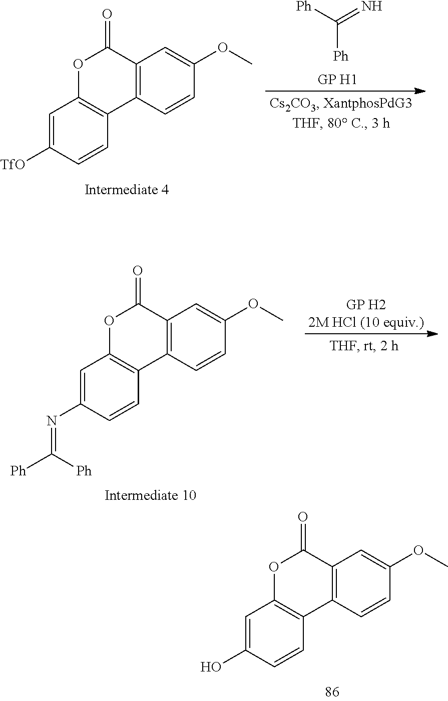 [SMILES: COc1ccc2c(c1)c(=O)oc1cc(OS(=O)(=O)C(F)(F)F)ccc12 ; N=C(c1ccccc1)c1ccccc1 ; COc1ccc2c(c1)c(=O)oc1cc(N=C(c3ccccc3)c3ccccc3)ccc12 ; COc1ccc2c(c1)c(=O)oc1cc(O)ccc12]

### GP H1

### 3-((Diphenylmethylene)amino)-8-methoxy-6H-benzo[c]chromen-6-one (Intermediate 10)

A glass vial was charged with a mixture of 8-methoxy-6-oxo-6H-benzo[c]chromen-3-yl trifluoromethanesulfonate (Intermediate 4) (300 mg, 0.802 mmol), benzophenone imine (0.20 mL, 1.20 mmol), cesium carbonate (392 mg, 1.20 mmol) and XPhos-Pd-G3 (76 mg, 0.080 mmol) in THF (4.0 mL). The reaction mixture was evacuated and purged with nitrogen (×3) and was heated at 80° C. for 2 h. The cooled mixture was partitioned between EtOAc (×2) and water and the combined organic extract was washed with brine, dried (MgSO₄) and concentrated in vacuo. The resultant residue was purified by chromatography on silica using 5-95% EtOAc in cyclohexane as eluant to give the product as a white solid (250 mg, 77%). ¹H NMR (400 MHz, DMSO-d₆) δ 8.23 (1H, d, J=9.0 Hz), 8.05 (1H, d, J=8.5 Hz), 7.61 (1H, d, J=2.8 Hz), 7.72-7.66 (2H, m), 7.53-7.46 (4H, m), 7.36-7.31 (2H, m), 7.33 (1H, ob. s), 7.27-7.21 (2H, m), 6.79 (1H, d, J=2.0 Hz), 6.73 (1H, dd, J=2.0, 8.4 Hz), 3.89 (3H, s). LCMS (Method 5): Rₜ=1.94 min; m/z=406.3 [M+H]⁺.

### GP H1

### 3-Amino-8-methoxy-6H-benzo[c]chromen-6-one (86)

A solution of 3-((diphenylmethylene)amino)-8-methoxy-6H-benzo[c]chromen-6-one (Intermediate 10) (250 mg, 0.617 mmol) in THF (3.0 mL) was treated with 2 M HCl (3.1 mL) and allowed to stir at RT for 10 mins. A precipitate was collected by filtration, which was then dissolved in MeOH and applied to a MeOH-equilibrated SCX-2 cartridge; after washing with MeOH/DCM the title compound was eluted using 7M NH₃ in MeOH to give the product as a beige solid (50 mg, 34%). ¹H NMR (400 MHz, DMSO-d₆) δ 8.15 (1H, d, J=8.8 Hz), 7.94 (1H, d, J=8.6 Hz), 7.61 (1H, d, J=2.8 Hz), 7.49 (1H, dd, J=2.8, 8.8 Hz), 6.67 (1H, dd, J=2.3, 8.6 Hz), 6.54 (11H, d, J=2.3 Hz), 5.82 (21H, s), 3.92 (31H, s); LCMS (Method 1): Rₜ=3.91 min; m/z=242.3 [M+H]⁺.

The following examples in Table A were prepared using similar methods those described above by utilizing the general procedures (GP) indicated.

TABLE A

| | | | | LCMS | HPLC Rₜ |
|---|---|---|---|---|---|
| | | General | ¹H NMR data | m/z | (min)/QC |
| Ex. | Structure | procedure | (400 MHz, DMSO-d₆) δ | (M + H) | Method |
| (87) | 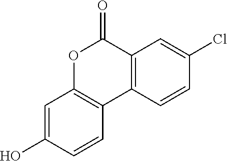 [SMILES: O=c1oc2cc(O)ccc2c2ccc(Cl)cc12] | A | 10.48 (1H, s), 8.36 (1H, d, J = 8.8 Hz), 8.23 (1H, d, J = 8.8 Hz), 8.17 (1H, d, J = 2.3 Hz), 7.98 (1H, dd, J = 2.4, 8.7 Hz), 6.91 (1H, dd, J = 2.4, 8.7 Hz), 6.82 (1H, d, J = 2.3 Hz) | 245.1, 247.1 | 4.52/1 |
| (92) | 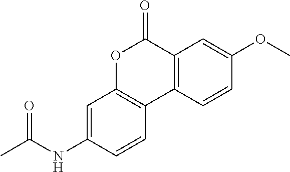 [SMILES: COc1ccc2c(c1)c(=O)oc1cc(NC(C)=O)ccc12] | A/D | 10.29 (1H, s), 8.24 (2H, dd, J = 8.9, 28.4 Hz), 7.78 (1H, d, J = 2.0 Hz), 7.64 (1H, d, J = 2.7 Hz), 7.55-7.46 (2H, m), 3.91 (3H, s), 2.10 (3H, s) | 284.0 | 3.72/3 |
| (94) | 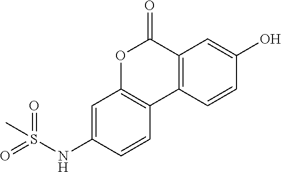 [SMILES: CS(=O)(=O)Nc1ccc2c(c1)oc(=O)c1cc(O)ccc12] | A/D/C2 | 10.38-10.35 (1H, m), 10.15 (1H, s), 8.21-8.17 (2H, m), 7.55 (1H, d, J = 2.6 Hz), 7.36 (1H, dd, J = 2.7, 8.7 Hz), 7.20-7.17 (2H, m), 3.09 (3H, s) | 305.8 | 3.06/3 |
| (99) | 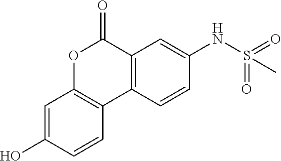 [SMILES: CS(=O)(=O)Nc1ccc2c(c1)c(=O)oc1cc(O)ccc12] | A/D2 | 10.29-10.19 (2H, m), 8.25 (1H, d, J = 8.7 Hz), 8.10 (1H, d, J = 8.9 Hz), 8.00 (1H, d, J = 2.4 Hz), 7.69 (1H, dd, J = 2.4, 8.7 Hz), 6.84 (1H, dd, J = 2.4, 8.7 Hz), 6.76-6.75 (1H, m), 3.07-3.06 (3H, m) | 305.9 | 3.07/3 |
| (100) | 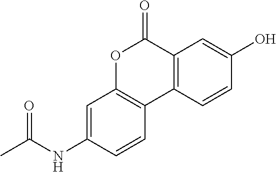 [SMILES: CC(=O)Nc1ccc2c(c1)oc(=O)c1cc(O)ccc12] | A/D/C2 | 10.27 (1H, s), 8.19-8.12 (2H, m), 7.75 (1H, d, J = 2.0 Hz), 7.54 (1H, d, J = 2.6 Hz), 7.46 (1H, dd, J = 2.0, 8.7 Hz), 7.35 (1H, dd, J = 2.7, 8.7 Hz), 2.09 (3H, s) | 269.9 | 2.94/3 |
| (101) | 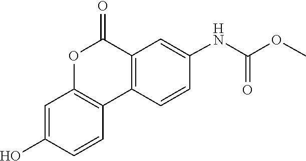 [SMILES: COC(=O)Nc1ccc2c(c1)c(=O)oc1cc(O)ccc12] | A/E | 10.26 (1H, s), 10.07 (1H, s), 8.35 (1H, d, J = 2.3 Hz), 8.20 (1H, d, J = 8.9 Hz), 8.07 (1H, d, J = 8.9 Hz), 7.92 (1H, dd, J = 2.4, 8.8 Hz), 6.83 (1H, dd, J = 2.4, 8.7 Hz), 6.74 (1H, d, J = 2.4 Hz), 3.71 (3H, s) | 285.9 | 3.38/3 |
| (102) | 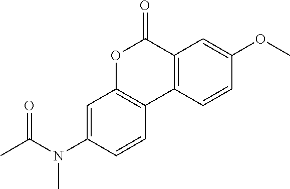 [SMILES: COc1ccc2c(c1)c(=O)oc1cc(N(C)C(C)=O)ccc12] | A/E | 8.41 (1H, d, J = 8.9 Hz), 8.35 (1H, d, J = 8.5 Hz), 7.68 (1H, d, J = 2.8 Hz), 7.57 (1H, dd, J = 2.8, 8.9 Hz), 7.50 (1H, s), 7.38 (1H, dd, J = 2.1, 8.5 Hz), 3.93 (3H, s), 3.24 (3H, s), 1.92 (3H, s) | 297.9 | 3.84/3 |
| (103) | 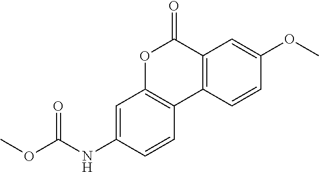 | A/E | 10.06 (1H, s), 8.27 (1H, d, J = 8.9 Hz), 8.20 (1H, d, J = 8.8 Hz), 7.64 (1H, d, J = 2.8 Hz), 7.58 (1H, d, J = 2.0 Hz), 7.53 (1H, dd, J = 2.8, 8.9 Hz), 7.42 (1H, dd, J = 2.1, 8.7 Hz), 3.91 (3H, s), 3.72 (3H, s) | 299.8 | 4.18/3 |
| (104) | 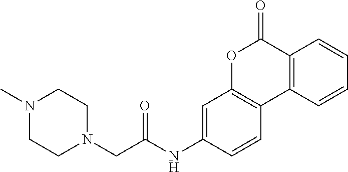 [SMILES: CN1CCN(CC(=O)Nc2ccc3c(c2)oc(=O)c2ccccc23)CC1] | A/D | 10.09 (1H, s), 8.35 (1H, d, J = 8.1 Hz), 8.30 (1H, d, J = 8.8 Hz), 8.23 (1H, dd, J = 1.0, 7.9 Hz), 7.96-7.91 (1H, m), 7.85 (1H, d, J = 2.0 Hz), 7.66-7.60 (2H, m), 3.17 (2H, s), 2.54 (br s, part obscured by solvent), 2.39 (4H, br s), 2.18 (3H, s) | 352.0 | 3.76/4 |
| (105) | 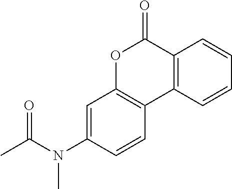 [SMILES: CC(=O)N(C)c1ccc2c(c1)oc(=O)c1ccccc12] | E | 8.46 (1H, d, J = 8.1 Hz), 8.42 (1H, d, J = 8.3 Hz), 8.27 (1H, dd, J = 1.2, 7.9 Hz), 8.01-7.95 (1H, m), 7.74-7.68 (1H, m), 7.52 (1H, d, J = 1.7 Hz), 7.41 (1H, dd, J = 2.2, 8.5 Hz), 3.25 (3H, s), 1.94 (3H, br S) | 268.0 | 3.77/4 |
| (106) | 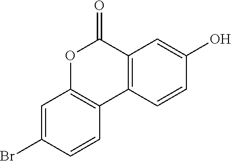 [SMILES: O=c1oc2cc(Br)ccc2c2ccc(O)cc12] | F/C2 | 10.5 (1H, s), 8.29 (1H, d, J = 8.8 Hz), 8.19 (1H, d, J = 8.6 Hz), 7.68 (1H, d, J = 2.0 Hz), 7.57 (1H, d, J = 2.7 Hz), 7.55 (1H, dd, J = 2.0, 8.6 Hz), 7.38 (1H, dd, J = 2.7, 8.8 Hz) | 288.9 | 4.02/4 |
| (107) | 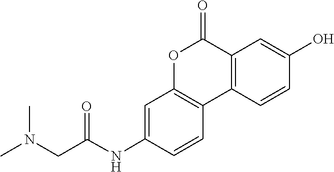 [SMILES: CN(C)CC(=O)Nc1ccc2c(c1)oc(=O)c1cc(O)ccc12] | F/D2 (from bromide Ex 35) | 10.34 (1H, s), 10.06 (1H, s), 8.19 (1H, d, J = 8.9 Hz), 8.15 (1H, d, J = 8.8 Hz), 7.84 (1H, d, J = 1.9 Hz), 7.63 (1H, dd, J = 1.9, 8.6 Hz), 7.55 (1H, d, J = 2.6 Hz), 7.35 (1H, dd, J = 2.6, 8.7 Hz), 4.34 (1H, t, J = 5.0 Hz), 3.48-3.40 (1H, m), 3.12 (2H, s), 2.29 (6H, s), 1.06 (1.8 H, t, J = 7.0 Hz) | 313.0 | 2.99/4 |
| (108) | 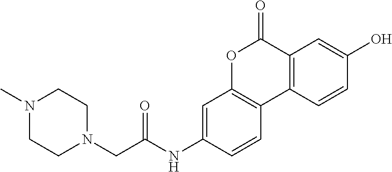 [SMILES: CN1CCN(CC(=O)Nc2ccc3c(c2)oc(=O)c2cc(O)ccc23)CC1] | F/D2 (from bromide Ex 35) | 10.34 (1H, s), 10.01 (1H, s), 8.20 (1H, d, J = 8.9 Hz), 8.16 (1H, d, J = 8.9 Hz), 7.81 (1H, d, J = 2.1 Hz), 7.59-7.53 (2H, m), 7.35 (1H, dd, J = 2.7, 8.8 Hz), 3.16 (2H, s), 2.53 (br s obscured by solvent), 2.40 (4H, br s), 2.18 (3H, s) | 368.0 | 2.27/3 |
| (108)* | 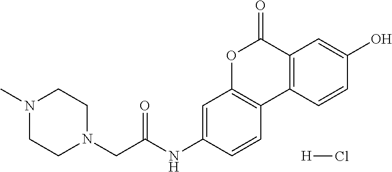 [SMILES: CN1CCN(CC(=O)Nc2ccc3c(c2)oc(=O)c2cc(O)ccc23)CC1 ; Cl] | F/D2 (from bromide Ex 35) | 11.30-10.20 (3H, m), 8.25-8.16 (2H, m), 7.82 (1H, d, J = 1.9 Hz), 7.58-7.53 (2H, m), 7.38, 1H, dd, J = 2.7, 8.8 Hz), 4.15-3.0 (br m, signals obscured by water), 2.80 (3H, s) | 368.0 | 2.80/4 |
| (111) | 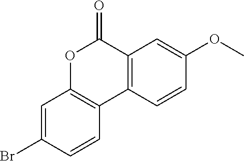 [SMILES: COc1ccc2c(c1)c(=O)oc1cc(Br)ccc12] | F | (CDCl₃) 8.00 (1H, d, J = 8.9 Hz), 7.84 (1H, d, J = 8.7 Hz), 7.80 (1H, d, J = 2.8 Hz), 7.53 (1H, d, J = 1.9 Hz), 7.46-7.39 (2H, m), 3.95 (3H, s) | 304.8, 306.8 | 5.30/3 |
| (85) | 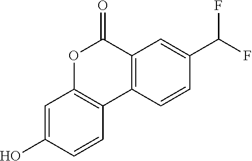 [SMILES: O=c1oc2cc(O)ccc2c2ccc(C(F)F)cc12] | A/G | (CDCl₃) 10.50 (1H, s), 8.41 (1H, d, J = 8.7 Hz), 8.35 (1H, d, J = 0.8 Hz), 8.21 (1H, d, J = 8.9 Hz), 8.06-8.02 (1H, m), 7.21 (1H, t, J = 55.5 Hz), 6.88 (1H, dd, J = 2.4, 8.7 Hz), 6.78 (1H, d, J = 2.4 Hz) | 260.9 | 4.03/3 |
| (112) | 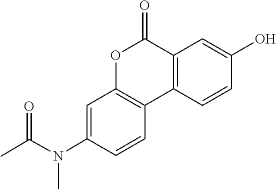 [SMILES: CC(=O)N(C)c1ccc2c(c1)oc(=O)c1cc(O)ccc12] | A/E | 10.46 (s, 1H), 8.32-8.26 (m, 2H), 7.58 (d, J = 2.6 Hz, 1H), 7.46 (s, 1H), 7.41-7.33 (m, 2H), 3.22 (s, 3H), 1.90 (s, 3H). | 283.9 | 3.06/3 |
| (113) | 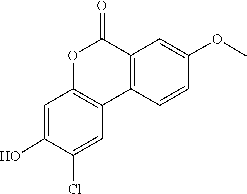 [SMILES: COc1ccc2c(c1)c(=O)oc1cc(O)c(Cl)cc12] | A | 11.05 (1H, br s), 8.35 (1H, s), 8.33 (1H, ob d J = 8.4 Hz), 7.64 (1H, d J = 2.8 Hz), 7.53 (1H, dd J = 2.8, 8.8 Hz), 6.97 (1H, s), 3.94 (3H, s) | 276.9 | 3.00/4 |

* = salt was prepared following treatment with 1.1 eq aqueous HCl and lyophilization. NMR spectra were obtained in d₆-DMSO unless otherwise stated.

### B) Ether and amide “A” group analogues

### Procedure I

### 2-Chloro-3,8-dihydroxy-6H-benzo[c]chromen-6-one (114) and 2-Chloro-6H-benzo[c]chromene-3,8-diol (115)

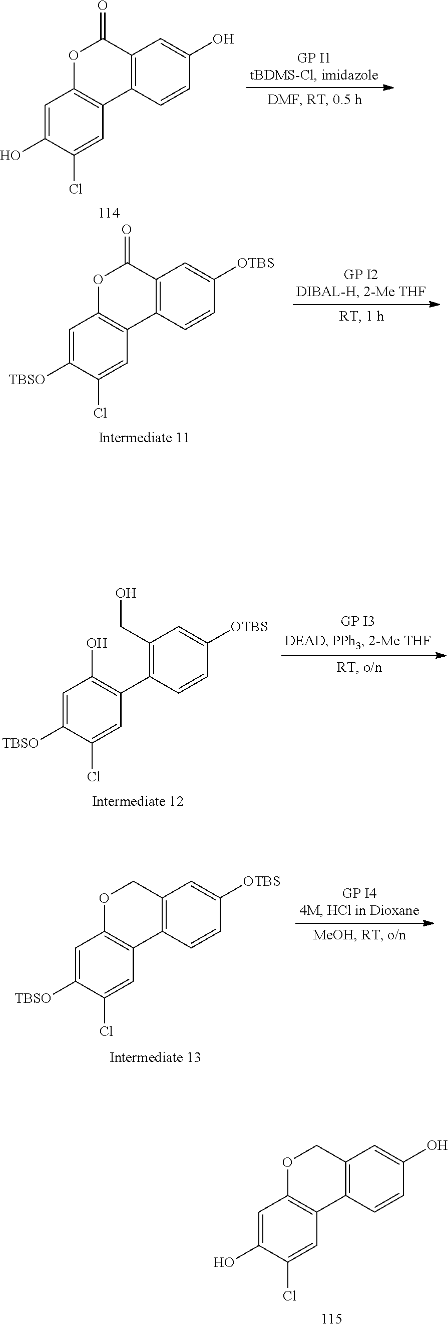 [SMILES: O=c1oc2cc(O)c(Cl)cc2c2ccc(O)cc12 ; CC(C)(C)[Si](C)(C)Oc1ccc2c(c1)c(=O)oc1cc(O[Si](C)(C)C(C)(C)C)c(Cl)cc12 ; CC(C)(C)[Si](C)(C)Oc1ccc(-c2cc(Cl)c(O[Si](C)(C)C(C)(C)C)cc2O)c(CO)c1 ; CC(C)(C)[Si](C)(C)Oc1ccc2c(c1)COc1cc(O[Si](C)(C)C(C)(C)C)c(Cl)cc1-2 ; Oc1ccc2c(c1)COc1cc(O)c(Cl)cc1-2]

### 2-Chloro-3,8-dihydroxy-6H-benzo[c]chromen-6-one (114)

2-Chloro-3,8-dihydroxy-6H-benzo[c]chromen-6-one was prepared from 113 using General Procedure C2. ¹H NMR (400 MHz, DMSO-d₆) δ 10.25 (1H, br s), 8.24 (1H, s), 8.19 (1H, d J=8.8 Hz), 7.51 (1H, d J=2.6 Hz), 7.31 (1H, dd J=2.7, 8.7 Hz), 6.91 (1H, s); LCMS (Method 3): Rₜ=3.52 min; m/z=260.9 [M−H]⁻.

### GP I1

### 3,8-bis((Tert-butyldimethylsilyl)oxy)-2-chloro-6H-benzo[c]chromen-6-one (Intermediate 11)

A suspension of 2-chloro-3,8-dihydroxy-6H-benzo[c]chromen-6-one (114) (2.37 g, 9.04 mmol) in DMF (15 mL) was treated with imidazole (2.46 g, 36.14 mmol) then TBDMSCl and the resulting mixture stirred at RT for 18 h. The reaction was partitioned between EtOAc (×3) and water and the combined organic extract was washed with brine, dried (PTFE frit) and concentrated in vacuo. The resultant residue was purified by chromatography on silica using 0-25% DCM in cyclohexane as eluant to give the product as a white solid (2.0 g, 45%). ¹H NMR (400 MHz, CDCl₃) δ 7.94 (1H, s), 7.85 (1H, d J=8.7 Hz), 7.76 (1H, d J=2.6 Hz), 7.31 (1H, dd J=2.7, 8.7 Hz), 6.89 (1H, s), 1.05 (9H, s), 1.01 (9H, s), 0.28 (6H, s), 0.26 (6H, s).

### GP I2

### 4,4′-bis((Tert-butyldimethylsilyl)oxy)-5-chloro-2′-(hydroxymethyl)-[1,1′-biphenyl]-2-ol (Intermediate 12)

To a solution of 3,8-bis((tert-butyldimethylsilyl)oxy)-2-chloro-6H-benzo[c]chromen-6-one (Intermediate 11) (385 mg, 0.784 mmol) in 2-Me THF (10 mL) was added dropwise, DIBAL-H (1.0 M in THF; 1.60 mL, 1.60 mmol) and the resulting solution was stirred at RT for 1 h. The mixture was cooled in an ice bath then quenched by addition of 15% aqueous NaOH (0.1 mL) followed by water (0.16 mL). After stirring for 30 mins Na₂SO₄ was added and the resultant mixture stirred for 18 h at RT. The mixture was filtered through Celite® and the pad washed with DCM, and the combined organic layer concentrated in vacuo to give a yellow solid (388 mg, quant.). ¹H NMR (400 MHz, CDCl₃) δ 7.24 (1H, s), 7.04 (1H, d J=8.3 Hz), 6.98 (1H, s), 6.88 (1H, s), 6.79 (1H, d J=7.7 Hz), 6.42 (1H, s), 4.27 (2H, m), 1.23 (1H, m), 1.02 (9H, s), 0.98 (9H, s), 0.20 (6H, s), 0.19 (6H, s).

### GP I3

### ((2-Chloro-6H-benzo[c]chromene-3,8-diyl)bis(oxy))bis(tert-butyldimethylsilane) (Intermediate 13)

To a solution of 4,4′-bis((tert-butyldimethylsilyl)oxy)-5-chloro-2′-(hydroxymethyl)-[1,1′-biphenyl]-2-ol (Intermediate 12) (388 mg, 0.783 mmol) and triphenylphosphine (308 mg, 1.17 mmol) in 2-Me THF (5.0 mL) was added dropwise DEAD (0.18 mL), and the mixture was stirred for 30 min at RT. The resulting solution was concentrated in vacuo and purified by chromatography on silica using 0-50% EtOAc in cyclohexane as eluant to give the semi-pure product. LCMS analysis gave desired product plus ˜70% of the fully de-protected diol. The crude reaction mixture was taken on to the next stage without purification.

### GP I4

### 2-Chloro-6H-benzo[c]chromene-3,8-diol (115)

A solution of crude ((2-chloro-6H-benzo[c]chromene-3,8-diyl)bis(oxy))bis(tert-butyl dimethyl silane) (Intermediate 13) (0.783 mmol) in MeOH (5.0 mL) was treated with 4 M HCl in dioxane (1.96 mL, 7.83 mmol) and the reaction was allowed to stir at RT for 18 h. The resultant mixture was concentrated in vacuo, and the residue was partitioned between DCM (×2) and water. The combined organic extract was washed with brine, dried (Na₂SO₄) and concentrated in vacuo and the crude residue was purified by chromatography on silica using 0-50% EtOAc in cyclohexane as eluant to give the title compound as a pale-yellow solid (60 mg, 31% yield). ¹H NMR (400 MHz, DMSO-d₆) δ 10.22 (1H, br s), 9.60 (1H, br s), 7.66 (1H, s), 7.54 (1H, d J=8.3 Hz), 6.74 (1H, d J=7.5 Hz), 6.62 (1H, s), 6.53 (1H, s), 4.99 (2H, s); LCMS (Method 3): Rₜ=3.57 min; m/z=246.9 [M−H]⁻.

### Procedure J

### 3,8-Dihydroxy-2-methyl-6H-benzo[c]chromen-6-one (116) and 2,6,6-Trimethyl-6H-benzo[c]chromene-3,8-diol (117)

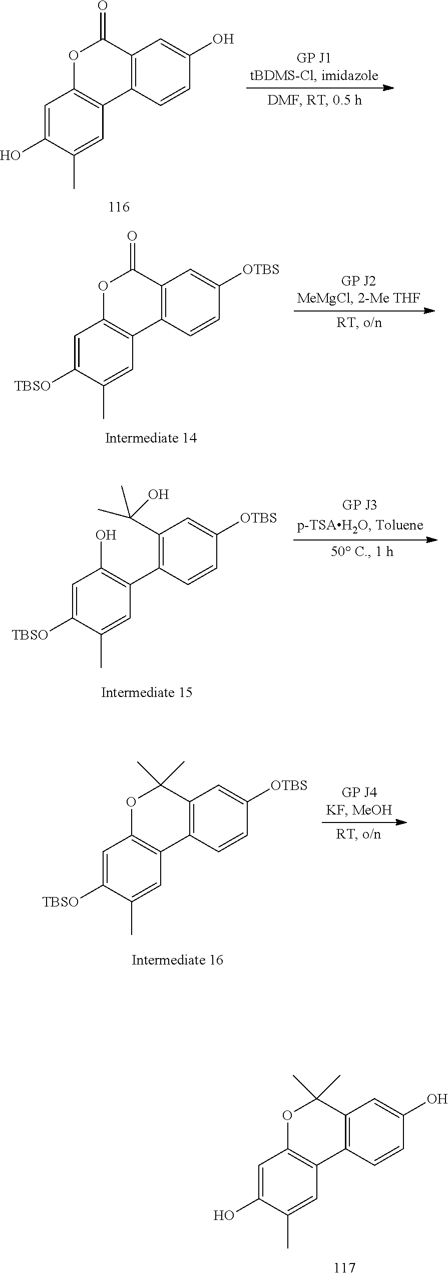 [SMILES: Cc1cc2c(cc1O)oc(=O)c1cc(O)ccc12 ; Cc1cc2c(cc1O[Si](C)(C)C(C)(C)C)oc(=O)c1cc(O[Si](C)(C)C(C)(C)C)ccc12 ; Cc1cc(-c2ccc(O[Si](C)(C)C(C)(C)C)cc2C(C)(C)O)c(O)cc1O[Si](C)(C)C(C)(C)C ; Cc1cc2c(cc1O[Si](C)(C)C(C)(C)C)OC(C)(C)c1cc(O[Si](C)(C)C(C)(C)C)ccc1-2 ; Cc1cc2c(cc1O)OC(C)(C)c1cc(O)ccc1-2]

### 3,8-Dihydroxy-2-methyl-6H-benzo[c]chromen-6-one (116)

3,8-Dihydroxy-2-methyl-6H-benzo[c]chromen-6-one was prepared using General Procedures A and C2. ¹H NMR (400 MHz, DMSO-d₆) δ 10.15 (2H, br s), 8.11 (1H, d J=8.9 Hz), 7.92 (1H, s), 7.50 (1H, d J=2.7 Hz), 7.31 (1H, dd J=2.7, 8.7 Hz), 6.74 (1H, s), 2.21 (3H, s); LCMS (Method 3): Rₜ=3.45 min; m/z=242.9 [M+1]⁺.

### GP J1

### 3,8-bis((Tert-butyldimethylsilyl)oxy)-2-methyl-6H-benzo[c]chromen-6-one (Intermediate 14)

3,8-bis((tert-butyldimethylsilyl)oxy)-2-methyl-6H-benzo[c]chromen-6-one was prepared from 3,8-dihydroxy-2-methyl-6H-benzo[c]chromen-6-one (120) using General Procedure I1 ¹H NMR (400 MHz, CDCl₃) δ 7.89 (1H, d J=8.7 Hz), 7.76 (1H, d J=2.7 Hz), 7.71 (1H, s), 7.28 (1H, dd J=2.6, 8.8 Hz), 6.78 (1H, s), 2.29 (3H, s), 1.03 (9H, s), 1.01 (9H, s), 0.27 (6H, s), 0.26 (6H, s).

### GP J2

### 4,4′-bis((Tert-butyldimethylsilyl)oxy)-2′-(2-hydroxypropan-2-yl)-5-methyl-[1,1′-biphenyl]-2-ol (Intermediate 15)

To a solution of 3,8-bis((tert-butyldimethylsilyl)oxy)-2-methyl-6H-benzo[c]chromen-6-one (Intermediate 14) (300 mg, 0.637 mmol) in 2-Me THF (6.0 mL) was added MeMgCl (3.0 M in THF; 0.64 mL, 1.92 mmol) and the resulting solution was stirred at RT for 18 h. The reaction mixture was quenched with saturated aqueous NH₄Cl and extracted with EtOAc (×2) prior to drying (Na₂SO₄) and concentrating in vacuo to give the title compound as a colourless oil (320 mg, quant.). ¹H NMR (400 MHz, CDCl₃) δ 7.10 (1H, d J=2.5 Hz), 6.94 (1H, d J=8.2 Hz), 6.86 (1H, s), 6.75 (1H, dd J=2.5, 8.2 Hz), 6.43 (1H, s), 5.08 (1H, s), 2.13 (3H, s), 2.04 (1H, s), 1.53 (3H, s), 1.42 (3H, s), 1.03 (9H, s), 1.01 (9H, s), 0.26 (3H, s), 0.25 (3H, s), 0.24 (6H, s).

### GP J3

### ((2,6,6-Trimethyl-6H-benzo[c]chromene-3,8-diyl)bis(oxy))bis(tert-butyldimethylsilane) (Intermediate 16)

A solution of 4,4′-bis((tert-butyldimethylsilyl)oxy)-2′-(2-hydroxypropan-2-yl)-5-methyl-[1,1′-biphenyl]-2-ol (Intermediate 15) (320 mg, 0.637 mmol) in toluene (5.0 mL) was treated with PTSA·H₂O and the resulting mixture was heated for 1 h at 50° C. The resulting solution was purified directly by chromatography on silica using DCM as eluant to give the product as a colourless oil (280 mg, 90%). ¹H NMR (400 MHz, CDCl₃) δ 7.48 (1H, d J=8.4 Hz), 6.38 (1H, s), 6.77 (1H, dd J=2.4, 8.4 Hz), 6.68 (1H, d J=2.4 Hz), 6.39 (1H, s), 2.19 (3H, s), 1.57 (6H, s), 1.02 (9H, s), 0.99 (9H, s), 0.23 (6H, s), 0.21 (6H, s). LCMS (Method 3): Rₜ=3.57 min; m/z=246.9 [M−H]⁻.

### GP J4

### 2,6,6-Trimethyl-6H-benzo[c]chromene-3,8-diol (117)

A suspension of ((2,6,6-trimethyl-6H-benzo[c]chromene-3,8-diyl)bis(oxy))bis(tert-butyl dimethylsilane) (Intermediate 16) (270 mg, 0.557 mmol) in MeOH (5.0 mL) was treated with solid KF (97 mg, 1.67 mmol) and the resulting suspension stirred at RT for 18 h. The resultant mixture was absorbed on to HMN and purified by chromatography on silica using 0-30% EtOAc in cyclohexane as eluant to give semi-pure product as a pale-yellow oil (121 mg). Further purification by trituration from a mixture of DCM and n-pentane gave the title compound as a white solid (91 mg, 64%). ¹H NMR (400 MHz, DMSO-d₆) δ 9.40 (1H, s), 9.32 (1H, s), 7.47 (1H, d J=8.4 Hz), 7.38 (1H, s), 6.71 (1H, dd J=2.3, 8.4 Hz), 6.66 (1H, d J=2.3 Hz), 6.31 (1H, s), 2.09 (3H, s), 1.50 (6H, s); LCMS (Method 3): Rₜ=3.80 min; m/z=257.1 [M+H]⁺.

Compound 117A was prepared by the below method.

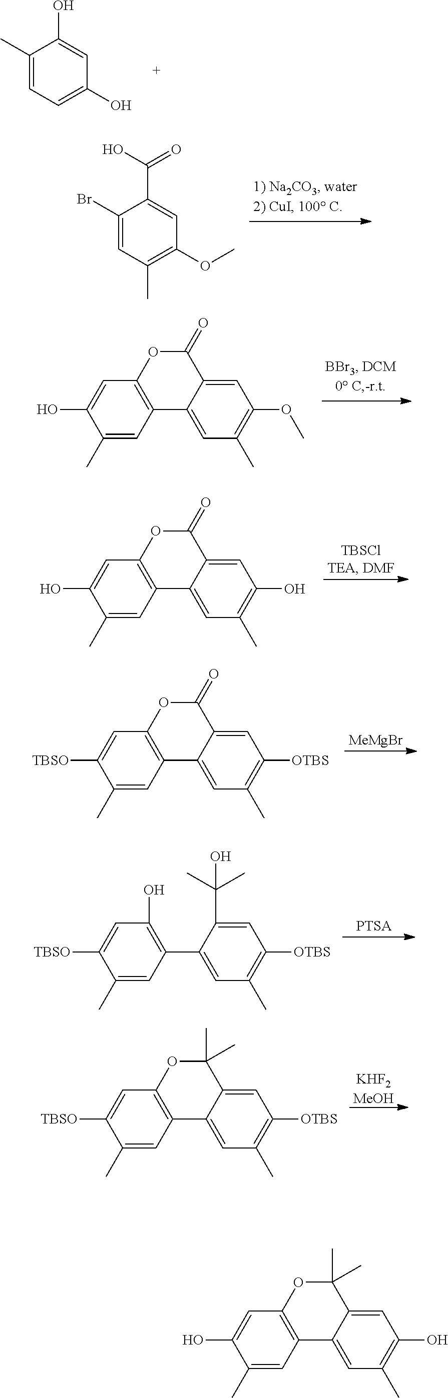

### Step 1: Synthesis of 3-hydroxy-8-methoxy-2,9-dimethyl-6H-benzo[c]chromen-6-one

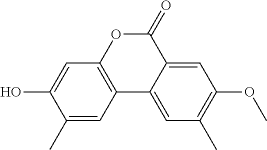 [SMILES: COc1cc2c(=O)oc3cc(O)c(C)cc3c2cc1C]

4-methylbenzene-1,3-diol (2.03 g, 16.3 mmol, 2.00 eq.) as well as Na₂CO₃ (2.60 g, 24.5 mmol, 3.00 eq.) were dissolved in water (10 mL) and upon complete dissolution 2-bromo-5-methoxy-4-methylbenzoic acid (2.00 g, 8.16 mmol, 1.00 eq.) was added in one portion and the mixture was heated to 60° C. in an oil bath for 1 h before adding CuI (777 mg, 4.08 mmol, 0.50 eq.) in one portion. Stirring at 60° C. was continued overnight before the reaction was allowed to cool to room temperature and filtered. The filter cake was suspended in 1M aq. HCl and filtered again. The remaining filter cake was dried at the high vacuum overnight to afford 3-hydroxy-8-methoxy-2,9-dimethyl-6H-benzo[c]chromen-6-one (1.27 g, 4.70 mmol, 58%) as a grey solid. R_{f}=0.30 (EtOAc/Cyclohexane 40%). ¹H NMR (400 MHz, DMSO) δ 10.12 (s, 1H), 8.08 (s, 1H), 7.95 (s, 1H), 7.52 (s, 1H), 6.73 (s, 1H), 3.90 (s, 3H), 2.33 (s, 3H), 2.21 (s, 3H).

### Step 2: Synthesis of 3,8-dihydroxy-2,9-dimethyl-6H-benzo[c]chromen-6-one

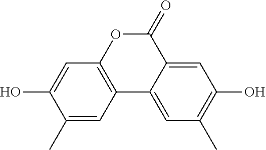 [SMILES: Cc1cc2c(cc1O)oc(=O)c1cc(O)c(C)cc12]

3-hydroxy-8-methoxy-2,9-dimethyl-6H-benzo[c]chromen-6-one (1.17 g, 4.30 mmol, 1.00 eq.) was suspended in DCM (44 mL) and cooled to 0° C. in an ice-bath. BBr₃ (13.0 mL, 13.0 mmol, 4.00 eq.) as a 1M solution in DCM was added dropwise. Upon complete addition the reaction was stirred at 0° C. for an additional 30 min before being allowed to warm up to room temperature. The reaction was stopped upon complete consumption of the starting material as indicated by TLC by quenching with methanol at 0° C. The methanol solution was concentrated under reduced pressure and the crude product was purified using flash column chromatography (0-10% MeOH in DCM) to afford 3,8-dihydroxy-2,9-dimethyl-6H-benzo[c]chromen-6-one (520 mg, 2.03 mmol, 47%) as a grey solid. R_{f}=0.2 (MeOH/DCM 10%). ¹H NMR (400 MHz, DMSO) δ 10.15 (s, 1H), 10.04 (s, 1H), 8.02 (s, 1H), 7.93 (s, 1H), 7.51 (s, 1H), 6.72 (s, 1H), 2.31 (s, 3H), 2.21 (s, 3H).

### Step 3: Synthesis 3,8-bis((tert-butyldimethylsilyl)oxy)-2,9-dimethyl-6H-benzo[c]chromen-6-one

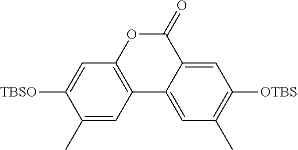 [SMILES: Cc1cc2c(cc1O[Si](C)(C)C(C)(C)C)oc(=O)c1cc(O[Si](C)(C)C(C)(C)C)c(C)cc12]

3,8-dihydroxy-2,9-dimethyl-6H-benzo[c]chromen-6-one (520 mg, 2.03 mmol, 1.00 eq.) was suspended in DMF (10 mL) and TBSCl (765 mg, 5.07 mmol, 2.50 eq.) were added in one portion. Following TEA (1.40 mL, 10.1 mmol, 5.00 eq.) was added dropwise and the reaction was stirred for 3 h. After that time the reaction was monitored by TLC and it was observed that due to solubility issues the reaction stalled and could not be pushed further. Therefore water was added to the reaction mixture and it was extracted with EtOAc (3×). The combined organic layers were dried over anhydrous Na₂SO₄, filtered and concentrated under vacuo. The crude was purified by flash column chromatography (0-10% EA in Hex) to afford 3,8-bis((tert-butyldimethylsilyl)oxy)-2,9-dimethyl-6H-benzo[c]chromen-6-one (290 mg, 0.60 mmol, 30%) as a white solid. R_{f}=0.4 (EtOAc/Cyclohexane 10%). ¹H NMR (400 MHz, CDCl₃) δ 7.78 (s, 1H), 7.71 (s, 1H), 7.66 (s, 1H), 6.78 (s, 1H), 2.39 (s, 3H), 2.29 (s, 3H), 1.04 (s, 9H), 1.03 (s, 9H), 0.30 (s, 6H), 0.27 (s, 6H).

### Step 4: Synthesis of 4,4′-bis((tert-butyldimethylsilyl)oxy)-2′-(2-hydroxypropan-2-yl)-5,5′-dimethyl-[1,1′-biphenyl]-2-ol

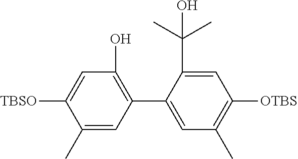 [SMILES: Cc1cc(-c2cc(C)c(O[Si](C)(C)C(C)(C)C)cc2C(C)(C)O)c(O)cc1O[Si](C)(C)C(C)(C)C]

3,8-bis((tert-butyldimethylsilyl)oxy)-2,9-dimethyl-6H-benzo[c]chromen-6-one (290 mg, 0.60 mmol, 1.00 eq.) was dissolved in THF (5 mL) and the reaction was cooled to 0° C. in an ice-bath. Following MeMgBr (0.60 mL, 1.79 mmol, 3.00 eq.) (3M in Et₂O) was added in one portion. The reaction was stirred at 0° C. for 10 min before being allowed to warm to room temperature. Stirring at room temperature was continued for 1 h before the reaction was quenched with water and extracted with EtOAc (3×).). The combined organic layers were dried over anhydrous Na₂SO₄, filtered and concentrated under vacuo. The crude was purified by flash column chromatography (0-20% EA in Hex) to afford 4,4′-bis((tert-butyldimethylsilyl)oxy)-2′-(2-hydroxypropan-2-yl)-5,5′-dimethyl-[1,1′-biphenyl]-2-ol (260 mg, 0.50 mmol, 84%) as a colorless oil which was directly used in the next step despite being an inseparable mixture with an undesired product.

### Step 5: Synthesis of ((2,6,6,9-tetramethyl-6H-benzo[c]chromene-3,8-diyl)bis(oxy))bis(tert-butyldimethylsilane)

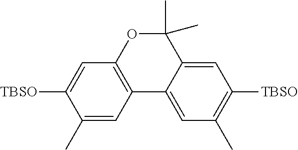 [SMILES: Cc1cc2c(cc1O[Si](C)(C)C(C)(C)C)OC(C)(C)c1cc(O[Si](C)(C)C(C)(C)C)c(C)cc1-2]

4,4′-bis((tert-butyldimethylsilyl)oxy)-2′-(2-hydroxypropan-2-yl)-5,5′-dimethyl-[1,1′-biphenyl]-2-ol (260 mg, 0.50 mmol, 1.00 eq.) was dissolved in toluene (5 mL) and PTSA (9.6 mg, 0.05 mmol, 10 mol %) were added. The mixture was heated to 70° C. for 10 min after which the starting material was completely converted. The mixture was concentrated under reduced pressure and the residue was directly subjected to flash column chromatography (0-10% EtOAc/Cyclohexane) to afford the desired ((2,6,6,9-tetramethyl-6H-benzo[c]chromene-3,8-diyl)bis(oxy))bis(tert-butyldimethylsilane) (175 mg, 0.35 mmol, 70%) as a yellowish solid. R_{f}=0.4 (EtOAc/Cyclohexane 10%). ¹H NMR (400 MHz, CDCl₃) δ 7.39 (d, J=6.2 Hz, 2H), 6.61 (s, 1H), 6.38 (s, 1H), 2.23 (s, 3H), 2.20 (s, 3H), 1.56 (s, 6H), 1.02 (s, 9H), 1.01 (s, 9H), 0.23 (s, 6H), 0.22 (s, 6H).

### Step 6: Synthesis of 2,6,6,9-tetramethyl-6H-benzo[c]chromene-3,8-diol

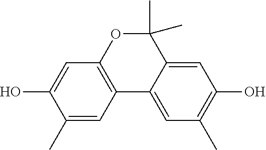 [SMILES: Cc1cc2c(cc1O)OC(C)(C)c1cc(O)c(C)cc1-2]

((2,6,6,9-tetramethyl-6H-benzo[c]chromene-3,8-diyl)bis(oxy))bis(tert-butyldimethylsilane) (175 mg, 0.35 mmol, 1.00 eq.) was dissolved in MeOH (5 mL) and 0.5 ml of DCM were added in order to completely dissolve the mixture. Then KHF₂ (110 mg, 1.40 mmol, 4.00 eq.) was added in one portion and the reaction was stirred overnight. After complete consumption of the stating material as indicated by TLC the reaction mixture was concentrated under reduced pressure and directly subjected to purification by flash column chromatography (0-5% MeOH in DCM) to yield 2,6,6,9-tetramethyl-6H-benzo[c]chromene-3,8-diol UA-0845 (40 mg, 0.15 mmol, 42%) as an orange solid. R_{f}=0.27 (MeOH/DCM 6%). ¹H NMR (400 MHz, DMSO) δ 9.28 (s, 1H), 9.25 (s, 1H), 7.38 (s, 2H), 6.65 (s, 1H), 6.29 (s, 1H), 2.14 (s, 3H), 2.09 (s, 3H), 1.45 (s, 6H). MS (APCI+): m/z=271.1.

### Procedure K

### 1-Fluoro-3,8-dihydroxyphenanthridin-6(5H)-one (118)

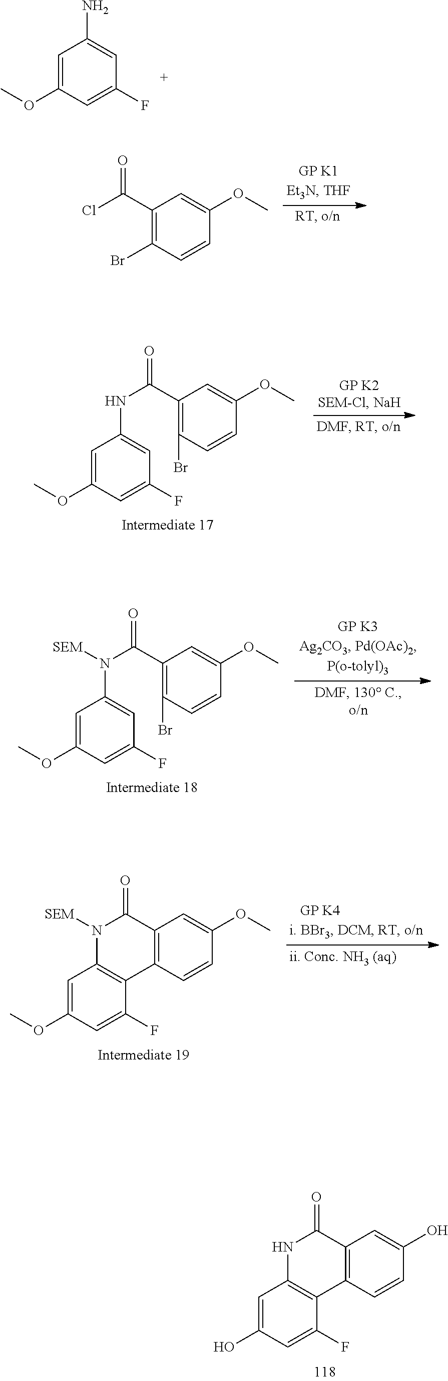

### GP K1

### 2-Bromo-N-(3-fluoro-5-methoxyphenyl)-5-methoxybenzamide (Intermediate 17)

To a solution of 2-bromo-5-methoxybenzoyl chloride (694 mg, 2.78 mmol) in THF (15 mL) was added triethylamine (0.58 mL, 4.17 mmol) followed by 5-methoxyaniline (393 mg, 2.78 mmol). The resulting solution was stirred at RT for 18 h then partitioned between water and DCM (×2) and the combined organic extract was dried and concentrated in vacuo. The residue was purified by chromatography on silica using 0-100% DCM in cyclohexane as eluant to give the title compound as a pale-yellow solid (700 mg, 71% yield). LCMS (Method 6): Rₜ=1.53 min; m/z=354.2/356.2 [M+H]⁺.

### GP K2

### 2-Bromo-N-(3-fluoro-5-methoxyphenyl)-5-methoxy-N-((2-(trimethylsilyl)ethoxy)methyl) benzamide (Intermediate 18)

A solution of 2-bromo-N-(3-fluoro-5-methoxyphenyl)-5-methoxybenzamide (Intermediate 17) (300 mg, 1.09 mmol) in dry DMF (3.0 mL) was treated with NaH (60 wt %; 87 mg, 2.18 mmol) and stirred at RT for 30 mins until gas evolution had ceased. SEM-Cl (0.72 mL, 3.27 mmol) was added to the reaction mixture and stirring was continued for 18 h at RT. The resulting solution was diluted with water (100 mL) then extracted with EtOAc (×3) and the combined organic extract was washed with brine then dried and concentrated in vacuo. The residue was purified by chromatography on silica using 0-30% EtOAc in cyclohexane as eluant to give the title compound as a pale-yellow solid (430 mg, 97% yield). LCMS (Method 6): Rₜ=1.89 min; m/z=482.3/484.3 [M+H]⁺.

### GP K3

### 1-Fluoro-3,8-dimethoxy-5-((2-(trimethylsilyl)ethoxy)methyl)phenanthridin-6(5H)-one (Intermediate 19)

A microwave vial was charged with a solution of 2-bromo-N-(3-fluoro-5-methoxyphenyl)-5-methoxy-N-((2-(trimethylsilyl)ethoxy)methyl) benzamide (Intermediate 18) (830 mg, 1.71 mmol) in dry DMF (8.0 mL). AgCO₃ (945 mg, 3.43 mmol), Pd(OAc)₂ (58 mg, 0.257 mmol) and tri(o-tolyl)phosphine (156 mg, 0.514 mmol) was added and the resultant mixture de-gassed under Argon prior to being heated for 18 h at 130° C. The cooled reaction mixture was filtered through Celite® then partitioned between water and EtOAc (×3) and the combined organic extract was washed with brine then dried and concentrated in vacuo. The resultant residue was purified by chromatography on silica using 0-50% EtOAc in cyclohexane as eluant to give the semi-pure product (270 mg). Further purification by trituration from a mixture of DCM and n-pentane gave purer material but an additional purification by chromatography on silica using 0-20% EtOAc in cyclohexane as eluant was required to give pure title compound as a white solid (186 mg, 27%). ¹H NMR (400 MHz, CDCl₃) δ 8.52 (1H, dd J=2.5, 9.1 Hz), 7.98 (1H, d J=3.0 Hz), 7.33 (1H, m), 7.04 (1H, m), 6.66 (1H, dd J=2.5, 14.9 Hz), 5.83 (2H, s), 3.95 (3H, s), 3.90 (3H, s), 3.76 (2H, t J=7.9 Hz), 0.97 (2H, t, J=8.1 Hz), −0.02 (9H, s).

### GP K4

### 1-Fluoro-3,8-dihydroxyphenanthridin-6(5H)-one (118)

A solution of 1-fluoro-3,8-dimethoxy-5-((2-(trimethylsilyl)ethoxy)methyl)phenanthridin-6(5H)-one (Intermediate 19) (86 mg, 0.213 mmol) in DCM (2.0 mL) was treated dropwise with BBr₃ and the resulting mixture was stirred for 18 h at RT. The reaction mixture was quenched carefully with water then azeotroped with MeOH (×4) which gave the intermediate hydroxy methyl amide, from partial de-protection of the SEM protecting group, as a yellow solid. LCMS (Method 6): Rₜ=1.09 min; m/z=276.0 [M+1]⁺.

The intermediate hydroxymethyl amide was treated with concentrated aqueous ammonia (3.0 mL) and the resultant turbid solution stirred for 3 h at RT then azeotroped with MeOH (×4) which gave the crude product as a grey solid. Further purification by reverse-phase chromatography on C₁₈-silica using 3-97% MeCN in water (+0.1% formic acid) gave pure title compound as a grey-white solid following lyophilisation (52 mg, 84%). ¹H NMR (400 MHz, DMSO-d₆) δ 11.60 (1H, br s), 10.05 (2H, br s), 8.28 (1H, d J=9.2 Hz), 7.66 (1H, d J=2.3 Hz), 7.25 (1H, m), 6.64 (1H, s), 6.49 (1H, d J=15.3 Hz); LCMS (Method 3): Rₜ=2.98 min; m/z=246.0 [M+1]⁺.

### Procedure L

### 6-Methyl-6H-benzo[c]chromene-3,8-diol (119)

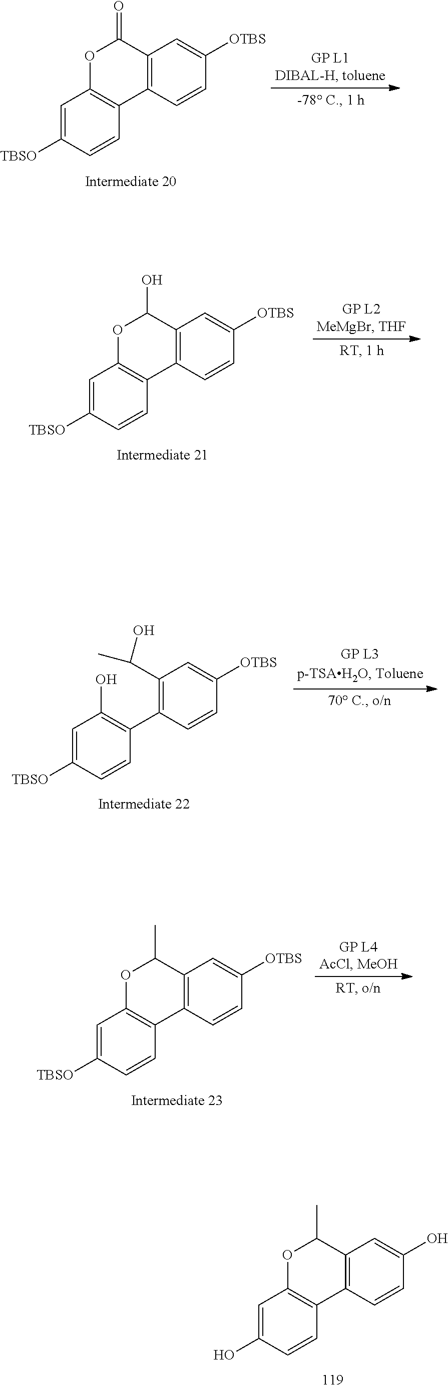 [SMILES: CC(C)(C)[Si](C)(C)Oc1ccc2c(c1)oc(=O)c1cc(O[Si](C)(C)C(C)(C)C)ccc12 ; CC(C)(C)[Si](C)(C)Oc1ccc2c(c1)OC(O)c1cc(O[Si](C)(C)C(C)(C)C)ccc1-2 ; CC(O)c1cc(O[Si](C)(C)C(C)(C)C)ccc1-c1ccc(O[Si](C)(C)C(C)(C)C)cc1O ; CC1Oc2cc(O[Si](C)(C)C(C)(C)C)ccc2-c2ccc(O[Si](C)(C)C(C)(C)C)cc21 ; CC1Oc2cc(O)ccc2-c2ccc(O)cc21]

### GP L1

### 3,8-bis((Tert-butyldimethylsilyl)oxy)-6H-benzo[c]chromen-6-one (Intermediate 20)

3,8-bis((Tert-butyldimethylsilyl)oxy)-6H-benzo[c]chromen-6-one was prepared using General Procedure I1. ¹H NMR (400 MHz, CDCl₃) δ 7.89 (d, J=8.8 Hz, 1H), 7.85-7.80 (m, 1H), 7.76 (d, J=2.6 Hz, 1H), 7.29 (dd, J=8.7, 2.7 Hz, 1H), 6.86-6.80 (m, 2H), 1.02 (s, 9H), 0.98 (s, 9H), 0.26 (s, 6H), 0.24 (s, 6H).

### GP L2

### 3,8-bis((Tert-butyldimethylsilyl)oxy)-6H-benzo[c]chromen-6-ol (Intermediate 21)

To a solution of 3,8-bis((tert-butyldimethylsilyl)oxy)-6H-benzo[c]chromen-6-one (Intermediate 20) (912 mg, 2.0 mmol) in toluene (20 mL) under inert atmosphere and at −78° C. was very slowly added DIBAL-H (1M in toluene, 2.10 mL, 2.10 mmol). Stirring was continued at −78° C. for 1 h. The reaction mixture was quenched by addition of water at −78° C. according to the Fieser work-up followed by the regular Fieser work-up. Filtration through silica and removal of the solvents in vacuo afforded the desired lactol as a white solid, which was used in the next step without further purification (921 mg, quant.). ¹H NMR (400 MHz, CDCl₃) δ 7.60 (dd, J=8.9, 6.9 Hz, 2H), 6.93 (dd, J=8.5, 2.5 Hz, 1H), 6.83 (d, J=2.6 Hz, 1H), 6.62-6.58 (m, 2H), 6.26 (s, 1H), 1.00 (s, 9H), 0.98 (s, 9H), 0.25-0.18 (m, 12H).

### GP L3

### 4,4′-bis((Tert-butyldimethylsilyl)oxy)-2′-(1-hydroxyethyl)-[1,1′-biphenyl]-2-ol (Intermediate 22)

To a solution of 3,8-bis((tert-butyldimethylsilyl)oxy)-6H-benzo[c]chromen-6-ol (Intermediate 21) (458 mg, 1 mmol) in dry THF (10 mL) under inert atmosphere and at 0° C. was added MeMgBr (3M in Et₂O, 1.0 mL). Stirring was continued at 0° C. for 1 h. The reaction mixture was quenched with water (100 mL) and the mixture extracted with Et₂O; the ethereal extracts were dried over Na₂SO₄, filtered through silica with Et₂O washings and then concentrated in vacuo. The crude residue was used in the next step without further purification (475 mg, quant.). ¹H NMR (400 MHz, CDCl₃) δ 7.15 (d, J=2.6 Hz, 0.4H), 7.12 (d, J=2.6 Hz, 0.6H), 7.07 (s, 0.4H), 7.04 (s, 0.6H), 6.96 (d, J=8.1 Hz, 0.4H), 6.90 (d, J=8.5 Hz, 0.6H), 6.86-6.78 (m, 1H), 6.52-6.43 (m, 2H), 4.79 (q, J=6.4 Hz, 0.4H), 4.73 (q, J=6.5 Hz, 0.6H), 1.36 (d, J=6.4 Hz, 1.2H), 1.30 (d, J=6.4 Hz, 1.8H), 1.01 (s, 7.2H), 1.00 (s, 10.8H), 0.25 (s, 4.8H), 0.24 (s, 7.2H).

### GP L4

### ((6-Methyl-6H-benzo[c]chromene-3,8-diyl)bis(oxy))bis(tert-butyldimethylsilane) (Intermediate 23)

To a solution of 4,4′-bis((tert-butyldimethylsilyl)oxy)-2′-(1-hydroxyethyl)-[1,1′-biphenyl]-2-ol (Intermediate 22) (470 mg, 0.98 mmol) in toluene (10 mL) was added PTSA monohydrate (19.0 mg, 0.19 mmol) and the resulting mixture was heated at 70° C. overnight. The reaction mixture was filtered through silica with DCM washings and concentrated in vacuo. The residue was purified by column chromatography (silica, 0-25% DCM/cyclohexane) to afford the desired ether as a white solid (411 mg, 90%). ¹H NMR (400 MHz, CDCl₃) δ 7.48 (dd, J=8.5, 4.1 Hz, 2H), 6.80 (dd, J=8.4, 2.5 Hz, 1H), 6.61 (dd, J=2.4, 0.8 Hz, 1H), 6.52 (dd, J=8.4, 2.4 Hz, 1H), 6.47 (d, J=2.4 Hz, 1H), 5.17 (q, J=6.5 Hz, 1H), 1.00 (s, 9H), 0.98 (s, 9H) 0.92-0.84 (m, 3H), 0.21 (s, 6H), 0.20 (s, 6H).

### GP L5

### 6-Methyl-6H-benzo[c]chromene-3,8-diol (119)

To a solution of ((6-methyl-6H-benzo[c]chromene-3,8-diyl)bis(oxy))bis(tert-butyldimethylsilane) (Intermediate 23) (411 mg, 0.90 mmol) in dry MeOH (10 mL) at 0° C. was slowly added acetyl chloride (96 mL, 1.35 mmol) and the resulting mixture was stirred overnight. The solvents were removed in vacuo and the residue purified by column chromatography (silica, 0-100% EtOAc/cyclohexane) to provide the title compound as a white solid (202 mg, 98%). ¹H NMR (400 MHz, DMSO) δ 9.47 (s, 1H), 9.45 (s, 1H), 7.49 (t, J=8.7 Hz, 2H), 6.73 (dd, J=8.4, 2.5 Hz, 1H), 6.60 (d, J=2.4 Hz, 1H), 6.43 (dd, J=8.4, 2.4 Hz, 1H), 6.30 (d, J=2.4 Hz, 1H), 5.14 (q, J=6.5 Hz, 1H), 1.44 (d, J=6.5 Hz, 3H).

The following examples in Table 2 were prepared using similar methods to those described above by utilizing the general procedures (GP) indicated.

TABLE 2

| | | | | | HPLC Rₜ |
|---|---|---|---|---|---|
| | | General | ¹H NMR data | LCMS m/z | (min)/QC |
| Ex. | Structure | procedure | (400 MHz, DMSO-d₆) δ | (M + H) | Method |
| (120) | 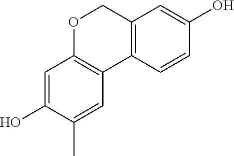 [SMILES: Cc1cc2c(cc1O)OCc1cc(O)ccc1-2] | I | 9.42 (2H, br s), 7.46 (1H, d J = 8.4 Hz), 7.39 (1H, s), 6.72 (1H, m), 6.59 (1H, s), 6.36 (1H, s), 4.91 (2H, s), 2.10 (3H, s) | | 3.46/3 |
| (121) | 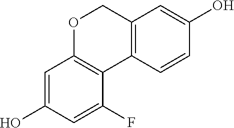 [SMILES: Oc1ccc2c(c1)COc1cc(O)cc(F)c1-2] | I | 9.79 (2H, br s), 7.60 (1H, d J = 8.1 Hz), 6.77 (1H, dd J = 2.5, 8.5 Hz), 6.67 (1H, d J = 2.4 Hz), 6.30 (1H, dd J = 2.3, 13.5 Hz), 6.24 (1H, m), 4.95 (2H, s) | 231.1 [M − H]⁻ | 3.41/3 |
| (122) | 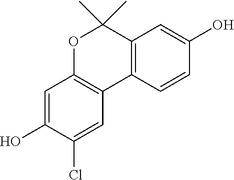 [SMILES: CC1(C)Oc2cc(O)c(Cl)cc2-c2ccc(O)cc21] | J | analytical data in progress | | |
| (123) | 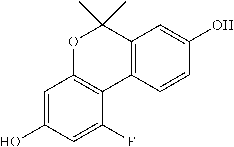 [SMILES: CC1(C)Oc2cc(O)cc(F)c2-c2ccc(O)cc21] | J | 9.76 (2H, v br s), 7.64 (1H, d J = 8.2 Hz), 6.78-6.72 (1H, obs m), 6.74 (1H, s), 6.27 (1H, dd J = 2.3, 13.7 Hz), 6.18 (1H, m), 1.50 (6H, s) | 259.0 [M − H]⁻ | 3.78/3 |
| (124) | 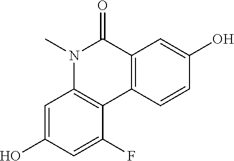 [SMILES: Cn1c(=O)c2cc(O)ccc2c2c(F)cc(O)cc21] | K | 10.18 (2H, br s), 8.32 (1H, d J = 9.2 Hz), 7.73 (1H, d J = 2.4 Hz), 7.25 (1H, m), 6.75 (1H, s), 6.62 (1H, d J = 15.3 Hz), 3.64 (3H, s) | 260.0 | 2.32/4 |
| (125) | 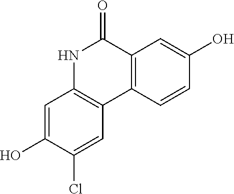 [SMILES: O=c1[nH]c2cc(O)c(Cl)cc2c2ccc(O)cc12] | K | 11.51 (1H, br s), 10.58 (1H, br s), 10.04 (1H, br s), 8.21 (1H, d J = 8.9 Hz), 8.19 (1H, s), 7.58 (1H, d J = 2.7 Hz), 7.21 (1H, d J = 2.7, 8.8 Hz), 6.95 (1H, s) | 259.9 [M − H]⁻ | 3.12/3 |
| (126) | 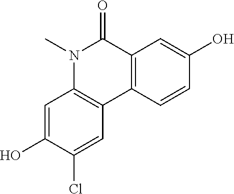 [SMILES: Cn1c(=O)c2cc(O)ccc2c2cc(Cl)c(O)cc21] | K | 10.52 (1H, v br s), 10.07 (1H, br s), 8.30 (1H, s), 8.26 (1H, d J = 8.9 Hz), 7.64 (1H, d J = 2.6 Hz), 7.22 (1H, d J = 2.6, 8.7 Hz), 7.04 (1H, s), 3.62 (3H, s) | 276.0 | 3.34/3 |
| (127) | 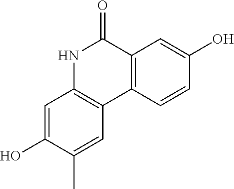 [SMILES: Cc1cc2c(cc1O)[nH]c(=O)c1cc(O)ccc12] | K | 11.34 (1H, s), 9.81-9.76 (2H, br s), 8.15 (1H, d, J = 9.0 Hz), 7.90 (1H, s), 7.57 (1H, d, J = 2.7 Hz), 7.20 (1H, dd, J = 2.7, 8.7 Hz), 6.77 (1H, s), 2.19 (3H, s) | 242.0 | 3.05/3 |
| (128) | 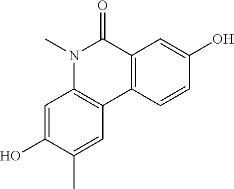 [SMILES: Cc1cc2c3ccc(O)cc3c(=O)n(C)c2cc1O] | K | 9.87 (2H, br s), 8.19 (1H, d J = Hz), 8.03 (1H, s), 7.64 (1H, d J = 2.7 Hz), 7.21 (1H, d J = 2.7, 8.8 Hz), 6.88 (1H, s), 3.62 (3H, s), 2.24 (3H, s) | 256.0 | 3.26/3 |
| (129) | 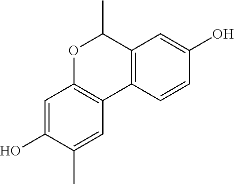 [SMILES: Cc1cc2c(cc1O)OC(C)c1cc(O)ccc1-2] | L | 9.57-9.26 (2H, br s), 9.59 (1H, br s), 7.47 (1H, d J = 8.4 Hz), 7.39 (1H, s), 6.73 (1H, dd J = 2.4, 8.4 Hz), 6.59 (1H, d J = 2.3 Hz), 6.35 (1H, s), 5.09 (1H, q J = 6.5 Hz), 2.10 (3H, s), 1.43 (1H, d J = 6.5 Hz) | 242.0 | 3.63/4 |
| (130) | 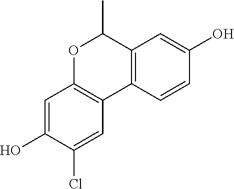 [SMILES: CC1Oc2cc(O)c(Cl)cc2-c2ccc(O)cc21] | L | 10.18 (1H, br s), 9.59 (1H, br s), 7.66 (1H, s), 7.55 (1H, d J = 8.5 Hz), 6.74 (1H, dd J = 2.5, 8.4 Hz), 6.61 (1H, d J = 2.4 Hz), 6.51 (1H, s), 5.18 (1H, q J = 6.5 Hz), 1.43 (1H, d J = 6.5 Hz) | 263.9 | 3.76/3 |
| (131) | 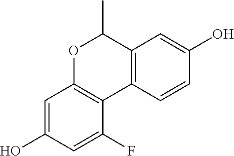 [SMILES: CC1Oc2cc(O)cc(F)c2-c2ccc(O)cc21] | L | 10.31-9.25 (2H, 2 x br s), 7.62 (1H, d J = 8.3 Hz), 6.76 (1H, dd J = 2.5, 8.5 Hz), 6.66 (1H, d J = 2.5 Hz), 6.28 (1H, dd J = 2.3, 13.6 Hz), 6.23-6.20 (1H, m), 5.18 (1H, q J = 6.5 Hz), 1.45 (1H, d J = 6.5 Hz) | 245.0 [M − H]⁻ | 2.87/4 |

NMR spectra were obtained in DMSO-d₆ unless otherwise stated.

### Procedure M

### 10-fluoro-3,9-dihydroxydibenzo[c,e]oxepin-5(7H)-one (132)

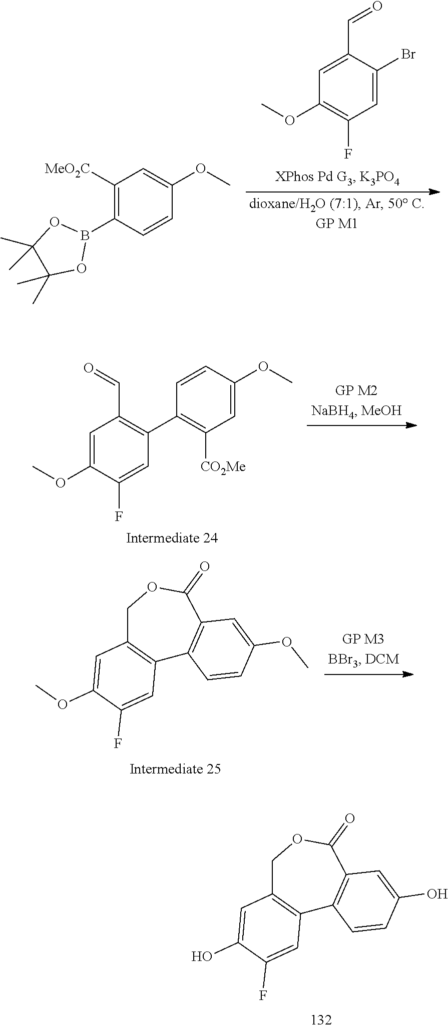

### GP M1

### Methyl 5′-fluoro-2′-formyl-4,4′-dimethoxy-[1,1′-biphenyl]-2-carboxylate (Intermediate 24)

A mixture of 2-bromo-4-fluoro-5-methoxybenzaldehyde (250 mg, 1.07 mmol), methyl 5-methoxy-2-(4,4,5,5-tetramethyl-1,3,2-dioxaborolan-2-yl)benzoate (345 mg, 1.18 mmol), and potassium phosphate (683 mg, 3.22 mmol) in dioxane (4 mL) and water (0.4 mL) was placed in a tube and degassed by purging with argon for 10 min. XPhos-Pd-G3 (45 mg, 0.05 mmol) was then added and the mixture was sealed, degassed and purged with argon. The mixture was heated at 50° C. for 1 h. The resulting cooled mixture was diluted with water then extracted with EtOAc (×3) and the combined organic extract was washed with brine then dried and concentrated in vacuo. The residue was purified by chromatography on silica using 0-25% EtOAc in cyclohexane as eluant to give the title compound as a pale-brown oil (280 mg, 82% yield). 1H NMR (400 MHz, CDCl₃) δ 9.66 (1H, s), 7.59 (1H, d, J=8.8 Hz), 7.55 (1H, d, J=2.8 Hz), 7.19 (1H, d, J=8.4 Hz), 7.14-7.08 (1H, m), 6.97 (1H, d, J=11.2 Hz), 3.99 (3H, s), 3.91 (3H, s), 3.67 (3H, s).

### GP M2

### 10-Fluoro-3,9-dimethoxydibenzo[c,e]oxepin-5(7H)-one (Intermediate 25)

To a solution of methyl 5′-fluoro-2′-formyl-4,4′-dimethoxy-[1,1′-biphenyl]-2-carboxylate (Intermediate 24) (270 mg, 0.85 mmol) in MeOH (4 mL) was added sodium borohydride (32 mg, 0.85 mmol) portionwise. The solution was stirred for 30 min then quenched with water and evaporated. The resulting mixture was diluted with water and EtOAc; this led to a large precipitate at the interface which was isolated by filtration and dissolved in a large volume of ethyl acetate and the combined organic extract was concentrated in vacuo. The solid residue was dissolved in CHCl₃/MeOH and evaporated to half volume, whereupon the product crystallised as a white solid; the mother liquors were adsorbed onto HMN and purified by chromatography on silica using 0-5% EtOAc in DCM as eluant to give additional product. The two batches of product were combined to give the title compound as a white solid (150 mg, 61% yield). 1H NMR (400 MHz, CDCl₃) δ 7.47 (1H, d, J=2.7 Hz), 7.44 (1H, d, J=8.6 Hz), 7.33 (1H, d, J=11.9 Hz), 7.21 (1H, dd, J=2.7, 8.8 Hz), 7.04 (1H, d, J=8.2 Hz), 5.03-4.89 (2H, m), 3.96 (3H, s), 3.91 (3H, s).

### GP M3

### 10-fluoro-3,9-dihydroxydibenzo[c,e]oxepin-5(7H)-one (132)

10-Fluoro-3,9-dimethoxydibenzo[c,e]oxepin-5(7H)-one (Intermediate 25) (145 mg, 0.5 mmol) was suspended in DCM (10 mL) and boron tribromide (2 mL. 1 M soln in DCM, 2.0 mmol) was added dropwise at RT. The resulting yellow solution was stirred overnight to give a yellow suspension. The mixture was cooled in ice-water and isopropanol (5 mL) was added dropwise to quench the reaction. The solvents were removed in vacuo. The resulting beige powder was dissolved in MeOH and adsorbed onto HMN and purified by chromatography on silica using 0-30% EtOAc in DCM to give the impure product which was re-purified on silica using 0-5% MeOH in DCM as eluant to give the title compound as an off-white solid (25 mg, 19%). ¹H NMR (400 MHz, DMSO-d₆) δ 10.59-9.60 (2H, m), 7.50 (1H, d, J=4.6 Hz), 7.47 (1H, d, J=8.3 Hz), 7.19 (1H, d, J=2.7 Hz), 7.16 (1H, d, J=8.9 Hz), 7.11 (1H, dd, J=2.7, 8.6 Hz), 5.10-4.74 (2H, m). LCMS (Method 3): Rₜ=2.96 min; m/z=259.0 [M−H]⁻.

### Synthesis of 9-ethylspiro[benzo[c]chromene-6,1′-cyclobutane]-3,8- (133)

### Step 1

Sodiumborohydride (756 mg, 20 mmol) was added portion wise to a stirred solution of 1-(4-bromo-2-hydroxyphenyl)ethan-1-one (4.3 g, 20 mmol) in 40 ml of methanol at 0° C. over 30 minutes. The reaction mixture was stirred at room temperature for 15 hours. Then the reaction mixture was quenched with acetone and solvent was evaporated. The residue was taken in water and extracted with ethyl acetate. The organic extracts were washed with water, brine, dried over anhydrous sodium sulphate and evaporated to afford crude 5-bromo-2-(1-hydroxyethyl)phenol which was taken for next step without any purification.

Trifluoroaceticacid (15 ml, 200 mmol) was added dropwise to a solution of 5-bromo-2-(1-hydroxyethyl)phenol (intermediate) (4 g), triethylsilane (3.19 ml, 20 mmol) in 25 ml of dichloromethane at 0° C. The reaction mixture was stirred at room temperature for 15 hour. Then the reaction mixture was evaporated under vacuum ad the crude was extracted with Na₂CO₃ sat sol and EtOAc twice. The organic solvent was dried over sodium sulfate and evaporated under vacuum. The crude was purified by FC eluent EtOAc/cyclohexane 0% to 7% to give 5-bromo-2-ethylphenol (2.2 g, 11 mmol, 55%) as yellowish oil which crystalizes at r.t. R_{f} 0.3 eluent EtOAc/cyclohexane 5%. ¹H NMR (400 MHz, CDCl₃) δ 7.01-6.99 (m, 2H), 6.95-6.92 (m, 1H), 4.83 (s, 1H), 2.58 (q, J=7.6 Hz, 2H), 1.21 (t, J=7.5 Hz, 3H).

### Step 2: Synthesis of 5-bromo-2-ethyl-4-iodophenol

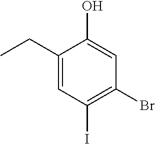

5-bromo-2-ethyl-4-iodophenol was prepared from 5-bromo-2-ethylphenol according to procedure described in Beatrice Felber, Francois Dietrich, Helvetica, 2005, vo188, 120-153.

### Step 3: Synthesis of 1-(benzyloxy)-5-bromo-2-ethyl-4-iodobenzene

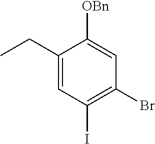 [SMILES: CCc1cc(I)c(Br)cc1OCc1ccccc1]

Benzyl Bromide (1.41 g, 9.17 mmol, 1 eq.) was added to a mixture of 5-bromo-2-ethyl-4-iodophenol (3 g, 9015 mmol) and potassium carbonate (2.5 g, 18.35 mmol, 2 eq.) in ACN (60 ml) and the mixture was heated at 50° C. for 3 h. The reaction mixture was filtered off concentrated under vacuum give 1-(benzyloxy)-5-bromo-2-ethyl-4-iodobenzene (3.5 g, 8.4 mmol, 91%) used to the next step without further purification. ¹H NMR (400 MHz, CDCl₃) δ 7.58 (d, J=0.7 Hz, 1H), 7.44-7.31 (m, 5H), 7.15 (s, 1H), 5.03 (s, 2H), 2.59 (qd, J=7.4, 0.7 Hz, 2H), 1.17 (t, J=7.5 Hz, 3H).

### Step 4: Synthesis of 4,4′-bis(benzyloxy)-2-bromo-5-ethyl-2′-fluoro-1,1′-biphenyl

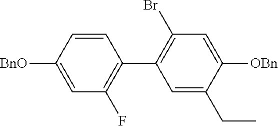 [SMILES: CCc1cc(-c2ccc(OCc3ccccc3)cc2F)c(Br)cc1OCc1ccccc1]

1-(benzyloxy)-5-bromo-2-ethyl-4-iodobenzene (1.5 g, 3.6 mmol) and (4-(benzyloxy)-2-fluorophenyl)boronic acid (1.3 g, 5.4 mmol, 1.5 eq.) were dissolved in dioxan (35 ml), Tetrakis(triphenylphosphine)palladium(o) (207 mg, 0.179 mmol, 0.05 eq.) was added and the solution was degassed then sodium bicarbonate solution (10.79 ml, 10.79 mmol, 3 eq.) was added and the mixture was heated at 90° C. overnight. NH4Cl saturated solution was added and the mixture was extracted with EtOAc twice. The combined organic phases were dried over sodium sulfate and concentrated under vacuum. The crude was purified by FC eluent EtOAc/cyclohexane 0% to 3% to give 4,4′-bis(benzyloxy)-2-bromo-5-ethyl-2′-fluoro-1,1′-biphenyl (1.5 g, 3.05 mmol, 84%). R_{f} 0.3 EA/cyclohexane 5%. ¹H NMR (400 MHz, CDCl₃) δ 7.49-7.33 (m, 10H), 7.22-7.16 (m, 2H), 7.10 (d, J=0.7 Hz, 1H), 6.85-6.81 (m, 1H), 6.80-6.76 (m, 1H), 5.09 (s, 4H), 2.67 (q, J=7.5 Hz, 2H), 1.21 (t, J=7.5 Hz, 3H).

### Step 5: Synthesis of 1-(4,4′-bis(benzyloxy)-5-ethyl-2′-fluoro-[1,1′-biphenyl]-2-yl)cyclobutan-1-ol

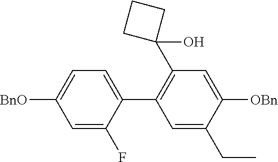 [SMILES: CCc1cc(-c2ccc(OCc3ccccc3)cc2F)c(C2(O)CCC2)cc1OCc1ccccc1]

n-BuLi (1M in THF) (1.37 ml, 2.19 mmol, 1.8 eq.) was added to a solution of 4,4′-bis(benzyloxy)-2-bromo-5-ethyl-2′-fluoro-1,1′-biphenyl (750 mg, 1.22 mmol) in THF (10 ml) at −78° C. The mixture was stirred for 30 min at −78° C. then cyclobutanone (684 mg, 9.78 mmol, 8 eq.) was added dropwise and the mixture was allowed to warm to rt. over 6 h. NH4Cl saturated solution was added and the mixture was extracted with EtOAc twice. The combined organic phases were dried over sodium sulfate and concentrated under vacuum. The crude was purified by FC eluent EtOAc/cyclohexane 0% to 20% to give 1-(4,4′-bis(benzyloxy)-5-ethyl-2′-fluoro-[1,1′-biphenyl]-2-yl)cyclobutan-1-ol (268 mg, 0.55 mmol, 45%). After purification the NMR still showed a mixture of products that could not be separated. However, this mixture was carried further to the next step where the desired product could eventually be isolated. Rf 0.25 EtOAc/cyclohexane 20%.

### Step 6: Synthesis of 3,8-bis(benzyloxy)-9-ethylspiro[benzo[c]chromene-6,1′-cyclobutane]

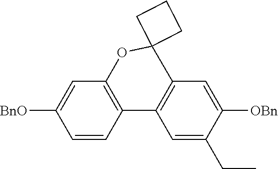 [SMILES: CCc1cc2c(cc1OCc1ccccc1)C1(CCC1)Oc1cc(OCc3ccccc3)ccc1-2]

Sodium hydride (62.2 mg, 1.55 mmol, 60% in oil) was added to a solution of 1-(4,4′-bis(benzyloxy)-5-ethyl-2′-fluoro-[1,1′-biphenyl]-2-yl)cyclobutan-1-ol (250 mg, 0.518 mmol) in DMF (5 ml) at r.t. The mixture was stirred overnight. NH4Cl saturated solution was added and the aqueous phase was extracted with EtOAC twice. The combined organic phases were dried over sodium sulfate and evaporated under vacuum. The crude was purified by FC eluent EtOAc/cyclohexane 0% to 5% to 10% to give 3,8-bis(benzyloxy)-9-ethylspiro [benzo[c]chromene-6,1′-cyclobutane] (150 mg, 0.324 mmole, 62%) as colorless oil. R_{f} 0.5 10% EA/Hexane. ¹H NMR (400 MHz, CDCl₃) δ 7.57 (d, J=8.4 Hz, 1H), 7.51-7.30 (m, 12H), 6.94 (s, 1H), 6.70-6.63 (m, 2H), 5.18 (s, 2H), 5.07 (s, 2H), 2.75 (q, J=7.5 Hz, 2H), 2.60-2.49 (m, 2H), 2.42-2.31 (m, 2H), 2.09-1.93 (m, 1H), 1.75 (dp, J=11.4, 8.7 Hz, 1H), 1.27 (t, J=7.5 Hz, 3H).

### Step 7: Synthesis of 9-ethylspiro[benzo[c]chromene-6,1′-cyclobutane]-3,8-diol

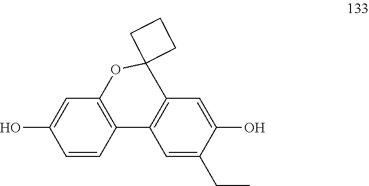 [SMILES: CCc1cc2c(cc1O)C1(CCC1)Oc1cc(O)ccc1-2]

3,8-bis(benzyloxy)-9-ethylspiro[benzo[c]chromene-1-6,1′-cyclobutane] (110 mg, 0.238 mmol) was dissolved in MeOH (4 ml). Nickel chloride hexahydrate (283 mg, 1.19 mmol 5 eq.) was added. NaBH4 (90 mg, 2.3 mmol, 10 eq.) were added portionwise (gaz evolution). TLC showed no more starting material. The mixture was filtered off and the crude was loaded on silica gel and purified by FC eluent MeOH/DCM 0% to 2% to 4% to give 9-ethylspiro[benzo[c]chromene-6,1′-cyclobutane]-3,8-diol UA-0852 (40 mg, 0.14 mmol 60%) as a white solid. R_{f} 0.4 eluent MeOH/DCM 4%. ¹H NMR (400 MHz, DMSO) δ 9.45 (s, 1H), 9.36 (s, 1H), 7.51 (d, J=8.5 Hz, 1H), 7.38 (s, 1H), 6.88 (s, 1H), 6.49-6.39 (m, 1H), 6.34 (d, J=2.4 Hz, 1H), 2.57 (q, J=7.5 Hz, 2H), 2.41 (ddd, J=12.6, 10.0, 8.5 Hz, 2H), 2.30-2.21 (m, 2H), 1.96 (dtt, J=14.2, 9.8, 4.2 Hz, 1H), 1.75 (dt, J=11.1, 8.7 Hz, 1H), 1.16 (t, J=7.5 Hz, 3H). MS (APCI+): m/z=283.1.

Additional compounds are prepared in accordance with methods adapted from the above procedures.

### Example 3: Urolithin A (UA)-induced mitophasgy promotes Wnt-mediated formation of T memory stem cells with superior antitumor immunity in colorectal cancer

Mice

Mice aged 6-30 weeks were maintained in a temperature controlled room with 12 h light and 12 h dark diurnal cycle. They were housed in filter-topped cages and fed standard laboratory chow and water ad libitum unless indicated otherwise. All animal procedures were performed in accordance with institutional guidelines. FvB and C57BL/6J mice were ordered from CharlesRiver and Janvier, while Rag1−/− and OT-1 mice were bred in-house. Pink1−/− mice (B6.129S4-PinkltmlShn/J; Strain #:017946) were purchased from The Jackson Laboratory. Pgam5−/− mice were a gift from C.B.

Urolithin A containing diet (2.28 g/kg) or AIN− 93G control food (Brogaarden) were given ad libitum once introduced for experimental purpose at the indicated time. Animal experiments were approved by the Regierungsprasidium Darmstadt under animal application numbers F123/1009; F123/1062 and F123/2008.

Human Samples

Human leukocytes were obtained from venous blood of mixed-sex donors aged 25-45 years after informed consent. CRC organoids were generated as part of the interdisciplinary Biobank and Database Frankfurt (iBDF) after prior written informed consent. The study was approved by the institutional review board of the UCT and the Ethical Committee at the University Hospital Frankfurt (Ethics vote: 4/09; project-numbers: SGI-06-2015 and SGI-17-2020).

In Vitro T Cell Isolation and Stimulation

Splenic murine T cells were isolated from mice aged 6-20 weeks by negative selection with EasySep mouse T cell isolation kit (Stem cell) according to the manufacturer's instructions. To monitor T cell proliferation, purified T cells were loaded with CellTrace Violet (C34557 ThermoFisher Scientific). T cell activation was initiated using αCD3/CD28 activation beads (11456D; ThermoFisher Scientific) at a ratio of 25 μl dynabeads per 0.5×10₆ T-cells. Cells were activated with 25-50 μM Urolithin A (Tocris) or DMSO in T cell activation Medium (RPMI (ThermoFisher Scientific) 10% FBS (South America origin), 10 mM Hepes (Sigma), 1×Non-Essential Amino Acid, 1 mM Sodium Pyruvate, 50 M β-Mercaptoethanol, 100 U/mL penicillin, 100 μg/mL streptomycin and 2 mM Glutamax (ThermoFisher Scientific) for the indicated durations. In selected experiments, cells were stimulated in the presence of inhibitors, namely TCFli (ICG-001; SelleckChem; 10 M) and PGC-lai (SR-18292, Sigma-Aldrich; 10 m). For subsequent analysis, activating beads were magnetically removed prior antibody staining. For human T cells, peripheral blood mononuclear cells were purified with a Ficoll-Paque (GE healthcare) gradient. T cells were enriched with EasySep Human T Cell Isolation Kit (StemCell) and activated with dynabead Human T-Activator CD3/CD28 for T Cell Expansion and Activation Kit (11131D; ThermoFisher Scientific), similar to murine T cell stimulation. When the subsequent activation and expansion of CAR T cells was studied, plates were coated with 1 μg/mL anti-human CD3 (αCD3; clone OKT3) and thawed PBMC were cultivated with 3 g/mL anti-human CD28 (αCD28; 15E8, both Miltenyi Biotec) containing media for three days. (NutriT media (4Cell® Nutri-T media (Sartorius) containing 0.5% penicillin/streptomycin) supplemented with IL-7 (25 U/mL) and IL-15 (U/mL) (all Miltenyi Biotec)).

Organoids

APTK colorectal organoids which harbor mutations for Apc, Trp53 and Tgfbr2 as well as expression of oncogenic KraSG12D were established and maintained as previously described (Nicolas et al., 2022; Varga et al., 2020). APTK organoids were seeded in 6 wells suspension plates: 6 droplets of 50 l Matrigel (Corning) per well were used. To generate OVA-APTK, colon organoids were isolated from unchallenged OVA mice (The Jackson Laboratory) as previously described (Nicolas et al., 2022). Liposomes CRISPR/Cas9 technology-based transfection was used for targeted knockout of the following mutated colorectal cancer-related genes: Apc (gRNA: TATGGAACCTGTCTGCACACTGCAC)(SEQ ID NO:1), Trp53 gRNA: GACACTCGGAGGGCTTCACT) (SEQ ID NO:2), and Tgfbr2 (gRNA: GGCCGCTGCATATCGTCCTG) (SEQ ID NO:3) as previously described (Schwank and Clevers, 2016). Cas9 plasmid (Addgene; #41815) was used along gRNA plasmids generated from addgene #41819 plasmid by cloning the gRNA of interest instead of GFP targeting gRNA. Firstly, Apc knockout (A) organoids were functionally selected by the withdrawal of R-spondin from the culture medium. Secondly, Apc Trp53 (AT) knockout organoids were selected for Trp53 loss with 5 μM of MDM2 inhibitor (Nutlin-3) (Biomole, cat no. 10004372). Thirdly, Apc Trp53 Tgfbr2 (APT) knockout organoids were selected with 5 μM recombinant TGFβ (R&D Systems; 7666-MB). Finally, KrasG12D gain of function mutation was obtained by overexpressing the active murine version of Kras gene (cloned in house based on Addgene plasmid #111164, Map-S5) in APT organoids. Apc, Trp53, Tgfbr2 and KrasG12D (APTK) OVA organoids were selected by 2 μg/mL puromycin (Merck; 58-58-2) and withdrawal of EGF and noggin from the culture medium. In order to study the effect of UA on lysosome formation, mitochondria reduction as well as antigen presentation, organoids were treated with 25-50 μM UA for 24-48 h. Organoids were subsequently stained with a MHC-I antibody (AF6-88.5; 116516; BioLegend), Mitotracker Deep Red (250 nm; ThermoFisher) and Lysotracker Red DND-99 (75 nM; ThermoFisher).

Human CRC organoids were established and cultured as previously described (Sato et al., 2011; van de Wetering et al., 2015). Organoids were cultured in medium composed of advanced DMEM/F12 supplemented with 20% R-spondin CM, 10% Noggin CM, 10 mM HEPES, 1×GlutaMax, 1× penicillin/streptomycin, 2% B27, 10 mM nicotinamide (Sigma-Aldrich), 12.5 mM N-acetylcystein (Sigma-Aldrich), 500 nM A83-01 (Tocris), 10 μM SB202190 (Biotrend), and 50 ng/mL human EGF (PeproTech). Unless stated otherwise, all media components were purchased from Life Technologies.

Cell Culture

Nalm-6 cells were cultivated in RPMI 1640 (Sigma, R₀₈₈₃-500 mL) medium supplemented with 10% fetal bovine serum (FBS; Sigma, F7524) and 2 mM glutamine (Sigma, G7513-100 mL). Their identity was confirmed by genetic phenotyping performed by the German cell culture collection (DSMZ).

Colorectal Cancer Models

Colorectal tumors were induced by 6 consecutive weekly injections of AOM on mice fed with food containing 2.28 g/kg Urolithin A (UA diet) or with control food. The diet was introduced one-week prior the first AOM injection and maintained until the end of the experiment at ˜20-24 weeks. The colons were collected for subsequent histological analysis as described below. For subcutaneous colorectal cancers, APTK organoids were mechanistically disrupted, incubated with Accutase to generate a single cell suspension and reconstituted in PBS 20% Matrigel (Corning) for subcutaneous transplantation. Tumor sizes were measured at the indicated time after transplantation and tumor volume quantified as ½(width₂×length). Urolithin A or control food regiment were initiated either one week before tumor challenge or 11 days after tumor development. Therapeutic diets were maintained throughout the experiment unless indicated otherwise. 200 g CD8-depleting antibodies (InvivoMab anti-mouse CD8; Hoelzel; BE0061) or isotype controls (InvivoMab rat IgG2b isotyp; Hoelzel; BE0090) were applied every three days, starting at five days after tumor initiation. To study combination efficacy with immunotherapy, mice were injected i.p. with 100 μl nVivoMAb anti-mouse PD-1 (Hoelzel; BE0146) and InVivoMab mouse IgG2b isotype control (Hoelzel; BE0086) every 2 days, starting three days after tumor injection.

Adoptive Cell Transfer

For adoptive cell transfer, purified splenic T cell from OT1 mice were isolated and activated using αCD3/αCD28 stimulation beads for 48 h. After magnetic bead removal, 1×10₆ T of activated T cell were intravenously injected into Rag1−/− recipients. To study survival of transferred T cells, stimulated OT-1 T cells were injected into mice without tumors. Subsequently, after 7 days, spleens were isolated and splenocytes were stained for the CD8+ subsets as described below. To understand antitumor immunity conferred by transferred OT-1 lymphocytes, cells were injected into mice carrying APTK-OVA tumors 10 days after subcutaneous organoid transplantation. Tumor growth was assessed as described above. Mice were kept under regular chow food for the T cell adoptive transfer experiment and sacrificed collectively when maximum tumor diameter extended 1.5 cm.

Lentiviral (LV) Production

Lentiviral vectors (LVs) were produced as described previously in detail for receptor targeted LVs (Weidner et al., 2021). In brief, HEK293T cells (ATCC CRL-11268) were transfected with a VSVG envelope (pMD2.G, Addgene: 12259), transfer and packaging plasmid in a ratio of 35:100:65 in presence of polyethylenimine (PEI; Sigma-Aldrich, 408727-100ML). The transfer vector plasmids encoded second generation CARs. The CEA-CAR encoding plasmid (Hombach et al., 2001) was modified by inserting the coding sequence for the reporter ΔLNGFR. The CD19-CAR contains a myc-tag for detection and was described (Pfeiffer et al., 2018). LVs were harvested from culture supernatants 48 h after transfection, concentrated by a 24 h centrifugation through a 20% sucrose cushion (Sigma, 84097) at 4,500×g (4° C.), and resuspended in Dulbecco's phosphate-buffered saline (DPBS, Mg2+ and Ca2+ free; Biowest, L0615-500). LVs were stored at −80° C. and thawed only once for each experimental use.

CAR Gene Delivery

For transduction, fully activated PBMC were seeded at 8×10₄ cells/well in NutriT media supplemented with IL-7 and IL-15 in a 96-well plate. 0.5 μL VSV-LV was added to the cells, following spinfection via centrifugation for 90 min at 850×g and 32° C. After spinfection, 100 μL of fresh media was added. Optionally, medium was replaced with fresh medium containing 25 M UA or DMSO 5 h post incubation with VSV-LV. Subsequently, cells were further cultivated at 37° C. Three days post transduction, CD19 CAR expression and the amount of T_{SCM} were determined. For CEA CAR T cells, T cells were frozen upon transduction and thawed for UA exposure experiments.

CAR T Cell Killing Assay

The cytotoxic activity of CD19-CAR T cells generated in presence or absence of UA was determined by CD19-positive Nalm-6 cell killing. Three days post transduction, CAR expression was determined and normalized to equal CAR T cell levels. Cells were washed twice and 0.5×10₄ CAR T cells were seeded into NutriT medium without cytokines, UA or DMSO. Nalm-6 cells were labeled using CellTrace™ Violet (CTV; Thermo Fisher Scientific, C34557) following manufacturer's instructions and 1×10₄ labeled Nalm-6 cells were added to the CAR T cells (0.5:1 effector to target ratio). The amount of dead target cells was determined via flow cytometry 24 h after start of killing.

In human CRC organoids, CEA-expression was confirmed by flow cytometry—APC (487609; FAB41281A; R&D Systems). Before co-culture, CEA-CAR T cells were cultivated in presence or absence of UA for three days. The cytotoxicity assay was performed as described (Schnalzger et al., 2019), using 96 well plates coated with 15 l Cultrex Basement Membrane Extract, Type 2 per well and organoids were seeded at a ratio of 1:8. Co-culture was performed for three days with 0.5×10₄ CAR T cells per well resuspended in organoid medium (as described above) without IL7/IL15. 100 l luciferase assay reagent composed of 10% One-Glo EX Reagent (Promega) in lysis buffer (50 mM NaCl, 50 mM Tris-HCl pH 7.4, 1% Triton X-100) was added to each well and incubated at room temperature for 1 h protected from light. 100 l of each sample was transferred to an opaque 96-well microtiter plate and measured using a SpectraMax iD3 microplate reader (Molecular Devices).

For freezing of CAR-T cells, pelleted cells were resuspended in 500 μl of NutriT medium supplemented with 20% FCS. 500 μl of NutriT medium supplemented with 20% FCS and 20% DMSO was added dropwise. For thawing, a vial of CAR T cells heated for 1 min in a 37° C. water bath. Thawed cells were washed twice with NutriT medium, centrifuged (5 min, 330 g) and resuspended in NutriT medium supplemented with IL-7 (25 U/mL) and IL-15 (U/mL).

Tumor Collection and Sample Preparation

Tumors were minced and digested with Tumor & Tissue Dissociation Reagent (BD Horizon) for 30 min. The remaining organ pieces were filtered through a 70 m cell strainer. Cells were then washed with PBS 1% BSA and 2 mM EDTA. Immune cells were enriched on a 30%/40%/75% Percoll gradient after a 1700 rpm 17 min centrifugation. Then, tumor immune filtrates or activated T cells were stained with Fixable Viability Dye-eF780 (65-0865-14; eBioscience), αCD45-FITC (30-F11; 11-0451-85; eBioscience), αCD11b-BV650 (M1/70; 56340; BD) α-CD11c-BV785 (HL3; 563735; BD), aLy6G-BV605 (53-6.7; 563005; BD), aLy6C-AF700 (HK1.4; 128024; Biolegend), aF4/80-PE (BM8; 12-4801-82, eBioscience), aPD-L1-PECy7 (MIH5; 25-5982-80, eBioscience); or with Fixable Viability Dye-eF780 (ebioscience), αCD45-BV786 (30-F11; 564225; BD), αCD4-BUV496 (GK1.5; 564667; BD), αCD8-BV605 (100744; Biolegend), aPD1-APC (J43; 17-9985-82; eBioscience), aTIM-3-PECy7 (RMT3-23; 119715; Biolegend), aCTLA4-PE (UC10-4B9; 106305; Biolegend) and aLag3-BV421 (C₉B7W; 125221, Biolegend); or with Fixable Viability Dye-eF780 (eBioscience), αCD4-FITC (H129.19; 553650; BD), αCD8-APC (53-6.7; 17-0081-83; eBioscience), αCD44-AF400 (IM7; 103026; Biolegend), αCD62L-BV785 (MEL-14; 104440; Biolegend), αSca-1-PerCP-Cy5.5 (D7; 45-5981-82; eBioscience), αCD95-PE (Jo2; 554258; BD); or with Fixable Viability Dye-eF780 (eBioscience), αCD8-BV650 (53-6.7; 563234; BD), αCD4-BUV496 (GK1.5; 564667; BD), αCD127-APC (A7R34; 17-1271-82; eBioscience), αCD95-FITC (SA367H8;152605; Biolegend), αCD44-AF400 (IM7; 103026; Biolegend), αCD62L-BV785 (MEL-14; 104440; Biolegend), αSca-1-PerCP-Cy5.5 (D7; 45-5981-82; eBioscience) followed by intracellular staining of aTCF1-PE (S33-966; 564217; BD), or PGC-1α (sc-518025; D-5; santa cruz), or aCyclinD1 (MA5-28534, DCS-6; Invitrogen) with transcription factor buffer set kit (BD Biosciences). To study cytokine expression, tumor immune infiltrates were stimulated ex vivo with 20 ng/mL PMA and 1 g/mL Ionomycin (Sigma) for 3 h in T cell activation medium with Brefeldin A (Biolegend). For T cell in vitro activation experiments over several days, Brefeldin A was added to the medium for the last 3 hours. Cells were then stained with Fixable Viability Dye-eF780 (eBioscience), αCD8-PeCy7 (53.6-7; 100722; Biolegend), αCD4-AF700 (GK1.5; 100429; Biolegend), and intracellular staining for cytokine assessment was performed with BD Cytofix/Cytoperm (BD) with 20 min fixation and overnight incubation with αGranzyme B-FITC (GB11; 515403; Biolegend), αIFNγ-PerCPCy5.5 (XMG1.2; 45-7311-82; eBioscience) and αTNFa-BV711 (MP6-XT22; 563944; BD). For flow cytometric analysis of mitochondria and lysosomes 500 μl of Mitotracker Red (200 nM; ThermoFisher) or Lysotracker Red DND-99 (75 nM; ThermoFisher) were applied followed by surface staining. To study changes in mitochondrial membrane potential, Mitoprobe TMRM kit (ThermoFisher) was used according to the manufacturer's instructions. Cell proliferation was assessed by CellTrace Violet (Invitrogen, C34557) at a concentration of 5 M according to manufacturer's instructions. For human samples, cells were stained as described above with Fixable Viability Dye-eF780, αCD95-BV421 (DX2; 562616), αCD62L-PE (DREG-56; 555544), αCCR7-AF700 (150503; 561143), αCD45RO-APC (UCHL; 559865), αCD45RAFITC (HI100; 555488), αCD8-PE-CF594 (RPA-T8; 562282) and αCD4-BV786 (OKT4; 566806) from BD.

For experiments studying activation, expansion and exhaustion of CAR T cells, cells were stained with αCD45RA-VioBlue (T6D11; 130-113-922; Miltenyi), αmycCAR-FITC (SH1-26e7.1.3; 130-116-653; Miltenyi), αCD8-PerCP-Cy5.5 (RPA-T8; 560662; BD), CD62L-PEVio770 (145/15; 130-113-621; Miltenyi), CD45RO-APC (UCHL1, 130-113-546; Miltenyi), Fixable Viability Dye-eF780; or mycCAR-FITC (SH1-26e7.1.3; 130-116-653; Miltenyi), αCD8-PerCP-Cy5.5 (RPA-T8; 560662; BD), aPD1-PE-Vio770 (PD1.3.1.3; 130-117-810; Miltenyi), αTim3-APC (REA635; 130-119-781; Miltenyi) and Fixable Viability DyeeF780. Finally, samples were fixed with 1% PFA until data acquisition.

Flow Cytometry Data Acquisition

Cell doublets and cells debris were excluded from the analysis by gating FSC-A vs FSC-H followed by SSC-A vs SSC-H dot plots and FSC-A vs SSC-A dot plots, respectively. eFluor 780 highly positive cells, i.e, dead cells, were gated out of the analysis as well as CD45 negative cells in the case of tumor samples. Data was acquired on Canto II (BD), Fortessa (BD) and Aurora (Cytek) cytometers and analyzed with FlowJo with the subsequent gating strategy described in the supplementary dataset.

Quantitative PCR with Reverse Transcription Analysis

In vitro activated T cells following the indicated treatments were collected followed by RNA isolation using the RNeasy kit (Qiagen). RNA quality and concentration were determined by Nanodrop spectrophotometer. Complementary DNA was generated using SuperScript II reverse transcriptase (ThermoFisher). A total reaction volume of 13 μl RNA was incubated with oligoDT and DNTP Mix (1 μl each) for 5 min at 65° C. The samples were allowed to cool down for 5 min on ice, and to each 4 μl of 5×First strand buffer, 1 μl DTT, 1 μl RNAseOUT™ and 1 μl Superscript II reverse transcriptase were added. Samples were incubated for 1 h at 42° C. Synthesized cDNA was 1:5 diluted in nuclease free water before being processed into gene expression analysis with the 96 wells StepOne real time PCR Machine (Applied biosystems) using 2× FastStart Universal SYBR Green Master Mix (Roche). CT values were normalized to the gene indicated in the respective figures.

The following primers were used:

Atg 5
(For: GGAGAGAAGAGGAGCCAGGT (SEQ ID NO: 4), Rev:
GCTGGGGGACAATGCTAATA (SEQ ID NO: 5)),
Atg7
(For: GCCTAACACAGATGCTGCAA (SEQ ID NO: 6), Rev:
TGCTCTTAAACCGAGGCTGT (SEQ ID NO: 7)),
Atg12
(For: TAAACTGGTGGCCTCGGAAC (SEQ ID NO: 8), Rev:
ATCCCCATGCCTGGGATTTG (SEQ ID NO: 9)),
Pik3c3
(For: GTGAAGTACCCTGACCTGCC (SEQ ID NO: 10), Rev:
AGTCATGCATTCCTTGGCGA (SEQ ID NO: 11)),
p62
(For: GCTGAAGGAAGCTGCCCTAT (SEQ ID NO: 12), Rev:
TTGGTCTGTAGGAGCCTGGT (SEQ ID NO: 13)),
Bnip3
(For: ATGCACAGCATGAGTCGGG (SEQ ID NO: 14), Rev:
CTGGTATGCATCTCAACATCAAACA (SEQ ID NO: 15)),
Lamp2
(TGTGCAACAAAGAGCAGGTG (SEQ ID NO: 16), Rev:
TCCAGTATGATGGCGCTTGAG (SEQ ID NO: 17)),
Pink1
(For: AAGCACCAGAAATTGCGACG (SEQ ID NO: 18), Rev:
ACGAGATGGGAGTGCTGGTA (SEQ ID NO: 19)),
TCF7
(For: CAATCTGCTCATGCCCTACC (SEQ ID NO: 20), Rev:
CTTGCTTCTGCGTGATGTCC (SEQ ID NO: 21)),
Rplp0
(For: TTCATTGTGGGAGCAGAC (SEQ ID NO: 22), Rev:
CAGCAGTTTCTCCAGAGC (SEQ ID NO: 23)),
Lef1
(For: GCAGCTATCAACCAGATCC (SEQ ID NO: 24), Rev
GATGTAGGCAGCTGTCATTC (SEQ ID NO: 25)),
Axin2
(For: CAGAGGTGGTACCTTGCCAAA (SEQ ID NO: 26), Rev
GCCGACAGTGCAAGACCC (SEQ ID NO: 27)),
cmyc
(For: AACTACGCAGCGCCTCCC (SEQ ID NO: 28), Rev:
ATTTTCGGTTGTTGCTGATCTGT (SEQ ID NO: 29)).

RNA Seq

For RNA isolation of in vitro stimulated T cells, RNeasy mini kit (Qiagen) was used. 200 ng of total RNA was used to prepare the library for RNA sequencing. Coexpression patterns of genes associated with effector and memory cell fate, immune checkpoints, effector molecules, and adhesion/homing were chosen based on the literature as depicted in the main text. Data underwent z-score normalization for display as described previously (Xiong et al., 2020). Upstream regulator analysis was performed using Ingenuity pathway analysis software, using a cut-off log 2 fold change of 1 and a p value of >0.05.

Imaging

Activated T cells were incubated on Poly-L-lysine coated coverslips for 10 min at 37° C. and then fixed with 4% PFA for 10 min, followed by permeabilization with 0.25% Triton X-100 for 10 mins. Staining for Pgam5 was performed as previously described (Yamaguchi et al., 2019), using rabbit Anti-Pgam5 antibody (ab126534, Abcam). Immune complexes were detected with 1:200-diluted anti-rabbit IgG Alexa Fluor 488 (Thermo Fisher). Nuclei were counterstained with a DAPI-containing liquid m liquid mountant (ProLong™ Gold Antifade Mountant, Thermo Fisher). Mitochondria were stained with 200 nM Mitotracker Red for 45 min Image were acquired by confocal microscopy.

Histology

For histological analysis of colon tissue, colons were paraffin embedded using the “swiss roll” technique and serially sectioned at 200 m. After deparaffinization and rehydration of the tissue, haematoxylin and eosin (H&E) staining and α-CD3 (IS503: DAKO) was performed using a Leica Bond Max following standard immunochemistry staining protocols. Stained sections were scanned using Aperio CS2 (Leica). AOM tumor lesion size and incidence were quantified using Aperio ImageScope software.

Protein Analysis

5×10₆ isolated T cells were stimulated on cell-culture plates coated with 1:200 α-CD3 (145-2C11; 100339; BioLegend) and α-CD28 (37.51; 102115, Biolegend) for the indicated time points. After centrifugation for 5 mins at 1500 rpm, cell pellets were resuspended in complete protein lysis buffer (50 mM Tris pH 7.5, 250 mM sodium chloride, 30 mM EDTA, 25 mM sodium pyrophosphate, 1% Triton-X 100, 0.5% NP-40, 10% glycerol and 1 mM DTT) supplemented with protease and phosphatase inhibitors (Roche). When subcellular location of Pgam5 was studied, a cell fractionation kit (Abcam, Ab109719) was used according to the manufacturer's instructions. Fractionation and whole-cell lysates were snap frozen and stored at −80° C. until further analysis. Collected lysates were mixed with 10% β-mercaptoethanol Laemmli buffer and denatured for 10 min at 70° C. Equal amounts of protein as assessed by Bradford measurement (Pierce™ Coomassie Plus (Bradford) Assay Reagent, ThermoFisher) were subjected to 10-15% SDS-PAGE, then transferred to 0.45 M PVDF membranes (MeckMillipore). Membranes were blocked for 30 mins in PBS-T/5% milk or PBST-T/5% BSA prior to antibody incubation overnight with the following antibodies: β-Actin (Sigma; #A4700), Gapdh (Santacruz; sc32233), Parkin (Santacruz; sc32283), Phospho-β-Catenin (CellSignaling; 9561), TOM20 (CellSignaling; D84TN;424065); pStat1 (Cellsignaling; 9167) and PGAM5 (Abcam; ab126534). Afterwards, membranes were incubated for one hour with their respective HRP-conjugated secondary antibodies (Santacruz: sc2314 and sc2313), followed by development using SuperSignal West Pico Chemoluminescence Substrate (ThermoFischer Scientific). 2D densitometry was performed using ImageJ Software (National Institutes of Health, Bethesda, Maryland).

Quantification and Statistical Analysis

All statistical analyses were performed using GraphPad Prism 8. Number of samples and experiments, as well as statistical test used are reported in each figure legend. Sample size was determined by prior experience. Data shown as ±SEM or ±SD according to figure legend. Statistical significance was assessed by the test depicted in the respective figure legend (*p<0.05, ** p<0.01, ***p<0.001 and ****p<0.0001)

Urolithin a Suppresses Intestinal Tumor Growth in a T Cell-Dependent Manner

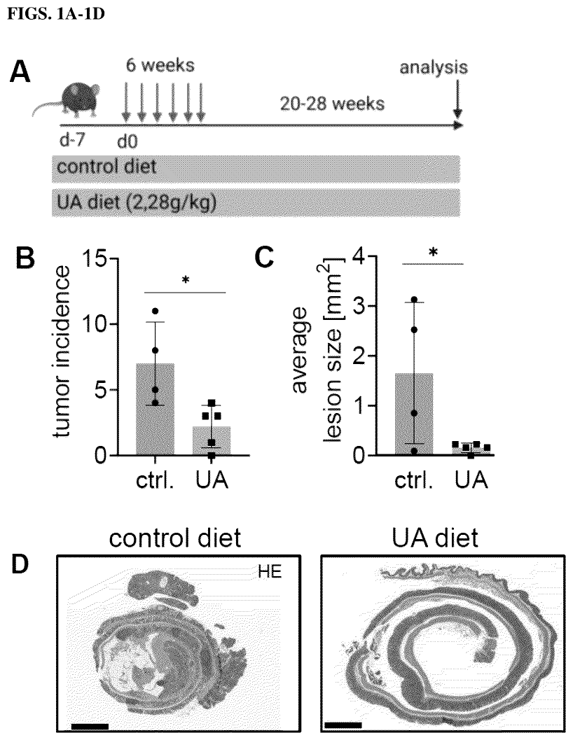

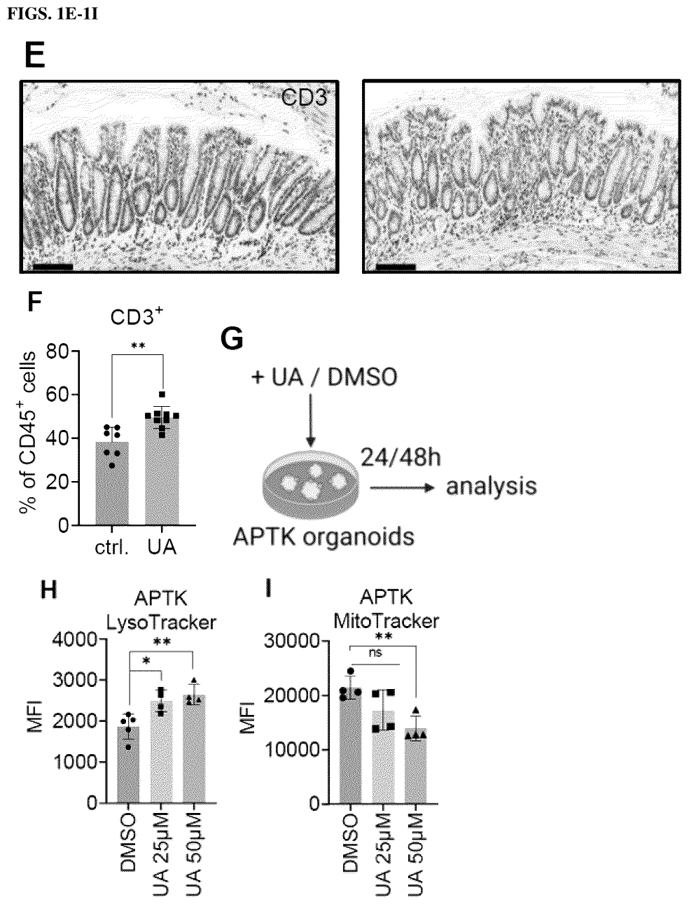

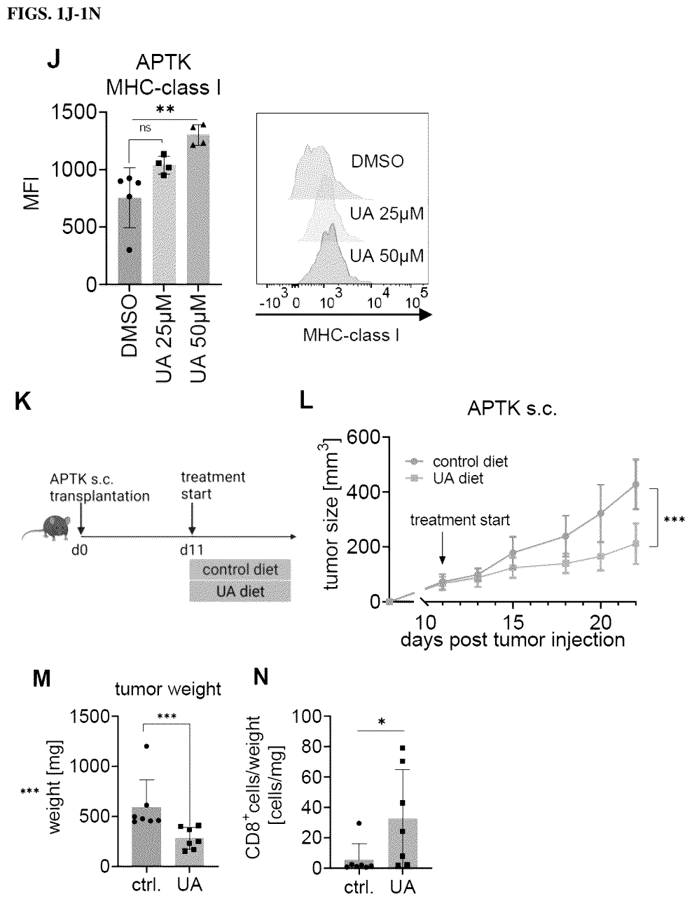

To examine whether Urolithin A (UA)-dependent mitophagy mimicked the effect of Stat3_{ΔIEC}mice (Ziegler et al., 2018) and prevented intestinal tumor development in a T cell dependent manner, a model of azoxymethane (AOM)-induced tumorigenesis was employed. FVB mice were injected with the procarcinogen AOM once a week for six weeks and kept for 18 weeks on an UA containing diet (2.28 g/kg) or control diet (FIG. 1A). Oral UA administration led to a significant decrease in tumor incidence and tumor size (FIG. 1B-D). Expectedly, this was accompanied by an increased infiltration of CD3+ T cells into the colonic mucosa (FIG. 1E, F).

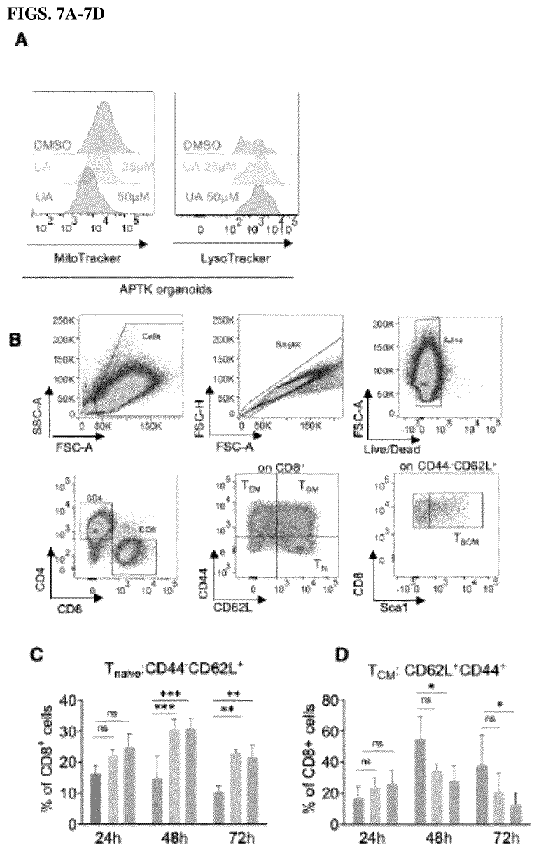

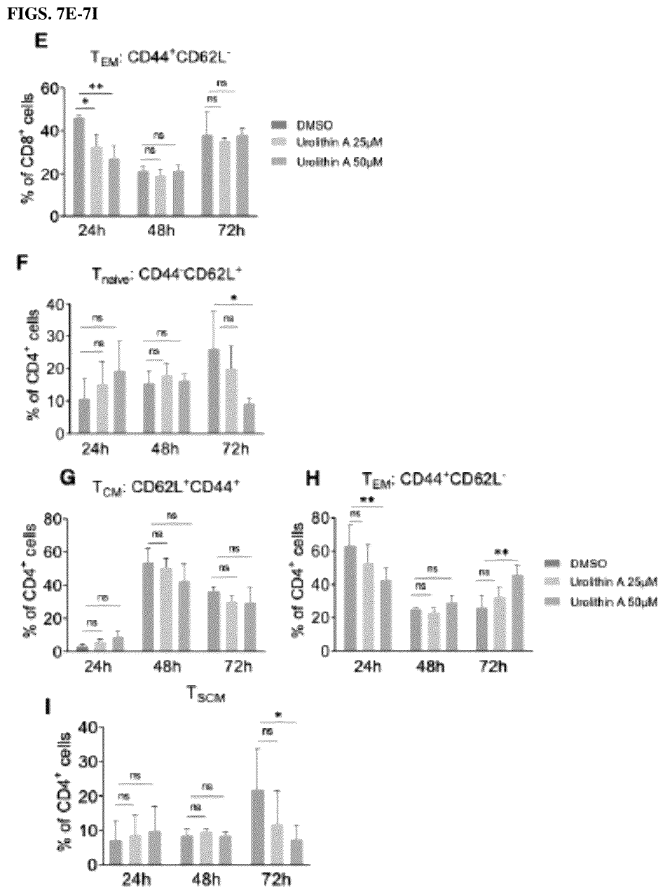

To confirm UA-induced mitophagy and to explore whether UA could be employed therapeutically in more advanced colorectal cancer, a recently developed tumor organoid system was used (Nicolas et al., 2022). We treated APTK organoids (characterized by loss of Apc, Trp53 and Tgfbr2 as well as expression of oncogenic Kras_{G12D}) for 48 hours with increasing concentrations of UA (FIG. 1G) and observed a dose-dependent formation of lysosomes (FIG. 1H, FIG. 7A) and a concomitant loss of MitoTracker staining (FIG. 1I, FIG. 7A). This was paralleled by a marked upregulation of MHC-I (FIG. 1J) validating our previous observation linking mitophagy in IEC to MHC-1 upregulation (Ziegler et al, 2018). To examine the therapeutic potential of UA, we subcutaneously (s.c.) transplanted APTK organoids into C57BL/6 mice (FIG. 1K). Tumor-bearing mice were subsequently subjected to the UA-supplemented diet, which resulted in significantly reduced tumor growth and enhanced CD8+ T cell infiltration (FIG. 1L-N). When we transplanted APTK organoids s.c. into Rag1−/− mice, the absence of mature T and B cells abrogated the protective effect of UA (FIG. 1O). Similarly, depletion of CD8+ T cells led to exacerbated tumor growth in both control and UA-fed cohorts (FIG. 1P-Q), confirming that UA-induced tumor suppression was T cell dependent. Consequently, we confirmed that UA sensitized APTK tumors to PD-1 blockade, while immune checkpoint inhibition alone did not affect APTK-tumor growth (FIG. 1R-S).

UA Promotes TSCM Differentiation

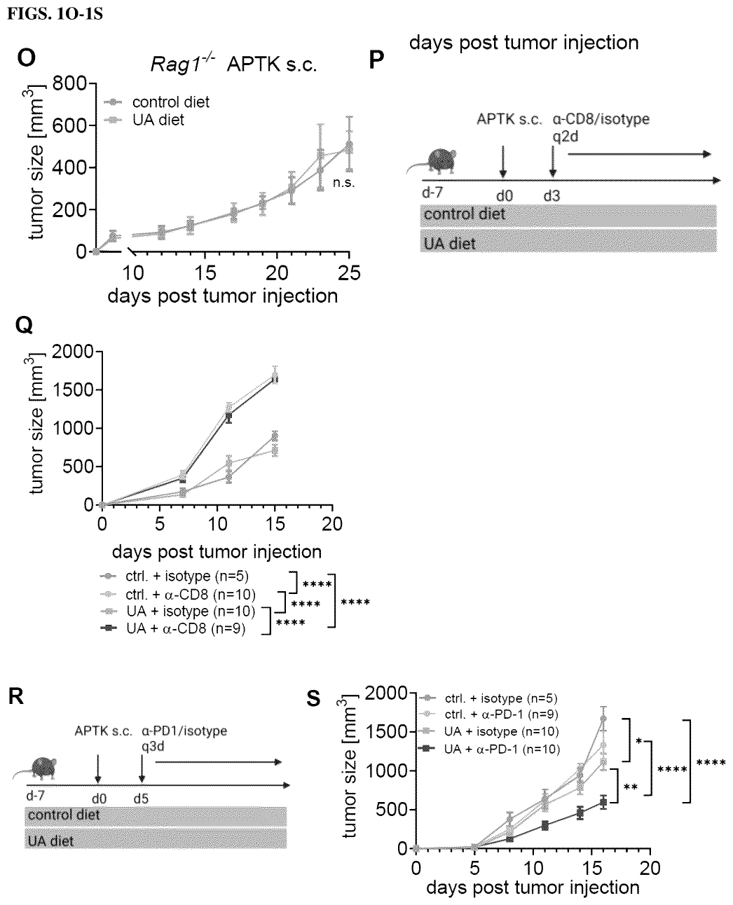

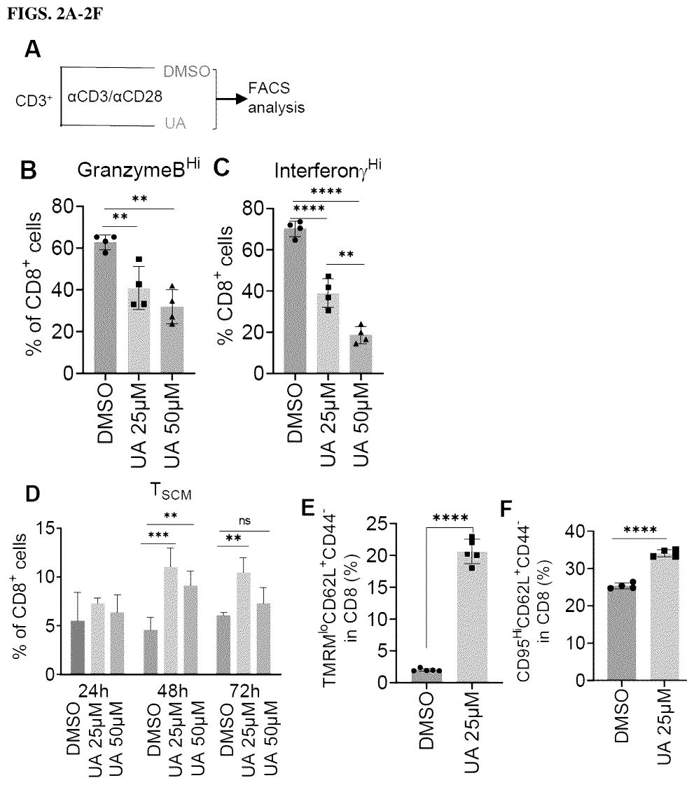

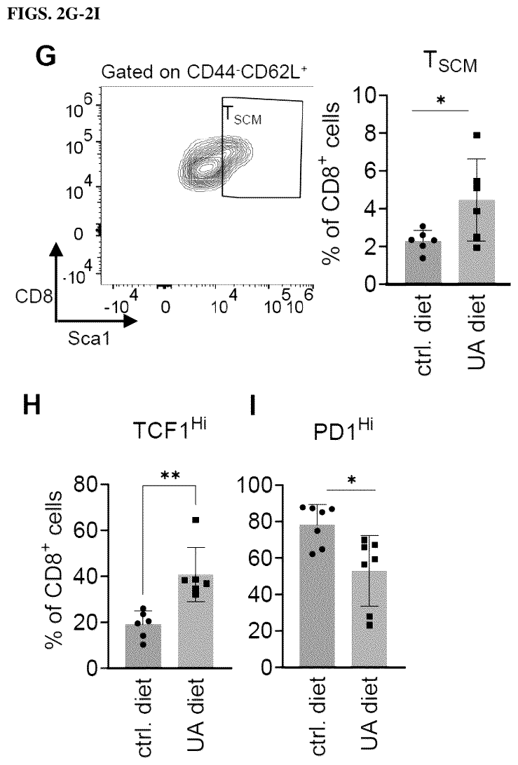

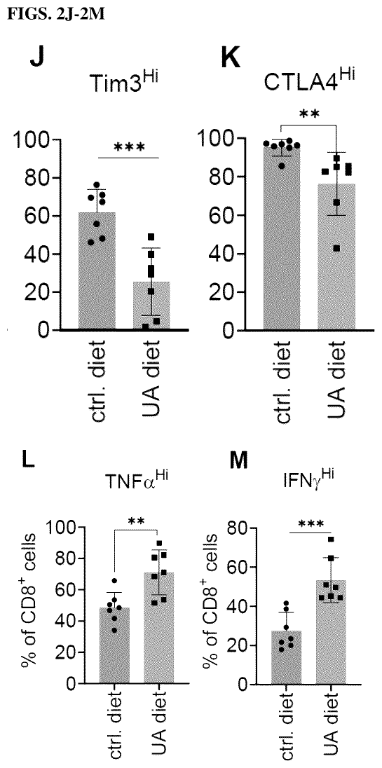

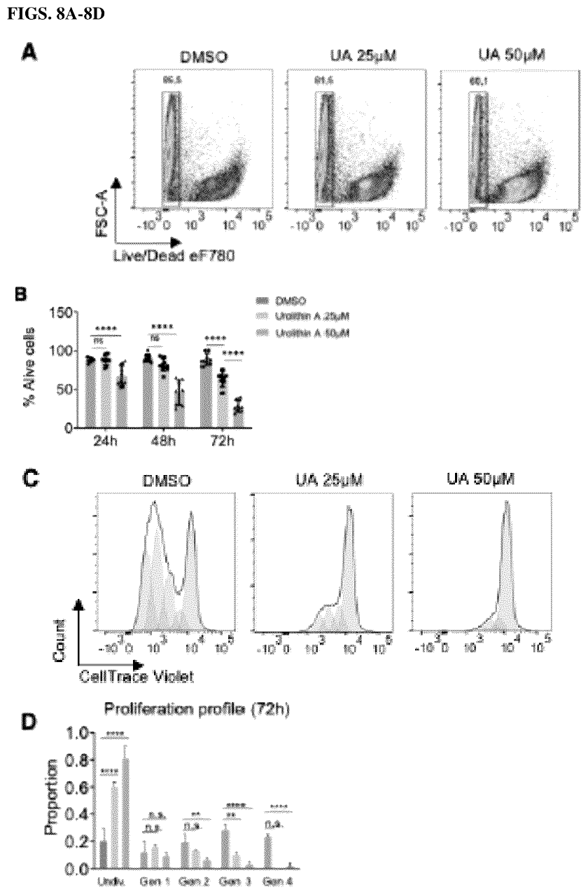

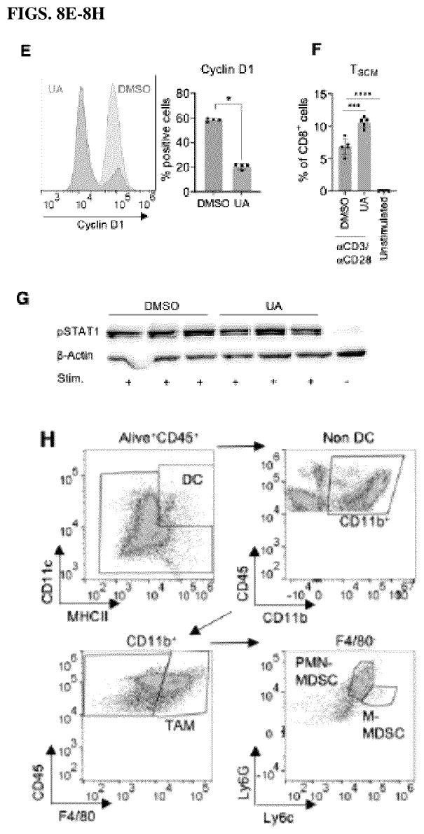

Mitochondrial remodelling has been previously associated with alterations in T cell fate (Buck et al., 2016; Yu et al., 2020b). Given the observed dependency of the UA effect on CD8+ T cells, it was analysed as to whether UA may also directly affect T cell fate commitment. Purified Tcells from FVB mice were stimulated with αCD3/αCD28 beads to induce T cell differentiation into effector T cell subsets in the presence or absence of UA for up to 72 hours (FIG. 2A). UA administration blocked differentiation into effector T cells (FIG. 2B, C) and resulted in a significant increase of naïve CD44ₗₒCD62Lₕᵢ T cells (FIG. 7B-C). In addition, a reduction of central memory cells (T_{CM}) was observed, whereas the frequency of effector memory cells (T_{EM}) remained unaltered (FIG. 7D-E). In CD4+ cells, we only observed changes at later timepoints, characterized by less naïve-like CD4+ cells, but enhanced frequency of T_{EM} cells (FIG. 7F-H). Previously, a rare naïve-like subset of T cells with enhanced stemness capabilities, termed T memory stem cells (T_{SCM}) has been identified (Gattinoni et al., 2009). T_{SCM} are marked by extreme longevity, their ability to self-renew, and potential for immune reconstitution (Gattinoni et al., 2017) which translates into potent anti-tumor immunity. Phenotypically, T_{SCM} represent a subset of minimally differentiated T cells, which share a CD44ₗₒCD62Lₕᵢ phenotype with naïve T cells, but are phenotypically distinct by expressing high levels of Sca1 (Gattinoni et al., 2009). Indeed, UA led to a significantly increased number of these CD44-CD62L+Sca1ₕᵢ T_{SCM} (FIG. 2D) that were characterized by a reduced mitochondrial membrane potential as well as increased CD95 expression (FIG. 2E, F). On the contrary, UA did not promote enhanced T_{SCM} formation in CD4+ cells (FIG. 7I). Furthermore, UA treatment led to dose-dependent T cell death in vitro (FIG. 8A-B) and restricted CD8+ T cell division (FIG. 2CD). This was associated with reduced cyclin D1 expression (FIG. 8E), in line with previous observations that link T_{SCM} reprogramming to halted proliferation (Gattinoni et al., 2009; Verma et al., 2021). Moreover, we observed that T_{SCM} were only formed in activated T cells (FIG. 8F). Excluding the possibility that UA reduces TCR-mediated activation of T cells, thus limiting effector formation by retaining a naïve-like state, we found that UA treated T cells show equal phosphorylation of Statl (Gamero and Lamer, 2000) compared to vehicle controls (FIG. 8G).

Also in vivo, UA administration led to a marked increase of CD8+CD44ₗₒCD62LₕᵢSca1ₕᵢ T cells in APTK-induced tumors (FIG. 2G), yet no change in CD4+T_{SCM} or in an antigen presenting or immune-suppressing TME represented by dendritic cells (DCs), TAMs, or MDSC subsets (FIG. 8H-M). This indicates that Urolithin A directly acts on T cells, and their reduced exhaustion state is not simply a by-product of an altered TME in this model. Indeed, fitting the observation of self-dividing memory subsets (Kratchmarov et al., 2018; Utzschneider et al., 2016), it was that found increased expression of the transcription factor 1 (TCF1) in tumor infiltrating CD8+ T cells (FIG. 2H). Additionally, it was observed a reduction ofexhaustion markers such as PD-1, CTLA-4 and Tim3 in CD8+ T cells of UA-fed mice (FIG. 2IK) which allowed stronger induction of TNFα and IFNγ when tumor infiltrating CD8+ T cells were restimulated with PMA/ionomycin ex vivo (FIG. 2L, M). Collectively, this data indicates that UA treatment results in the formation of T_{SCM} in the TME, conferring superior CD8+-mediated antitumor immunity.

UA Improves Tumor Therapy by Adoptive T Cell Transfer

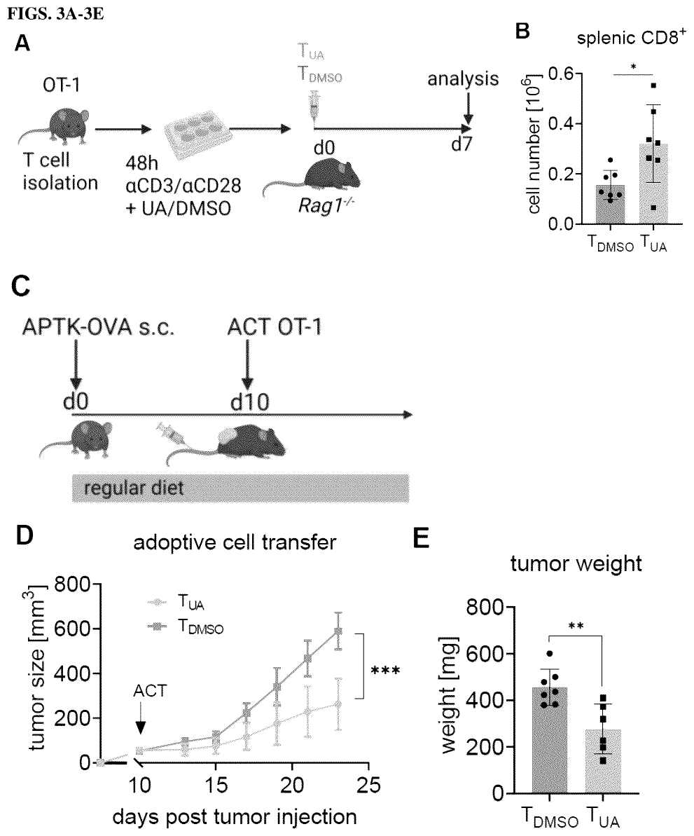

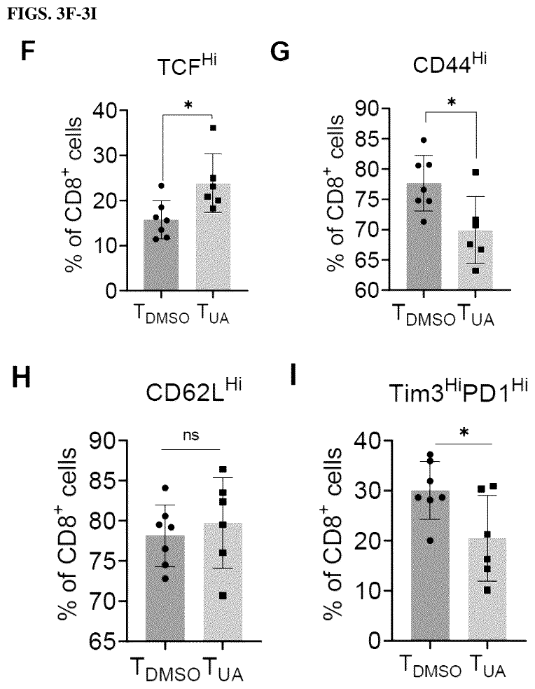

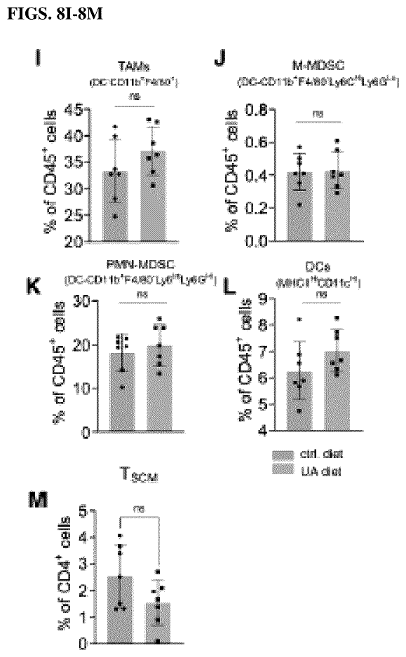

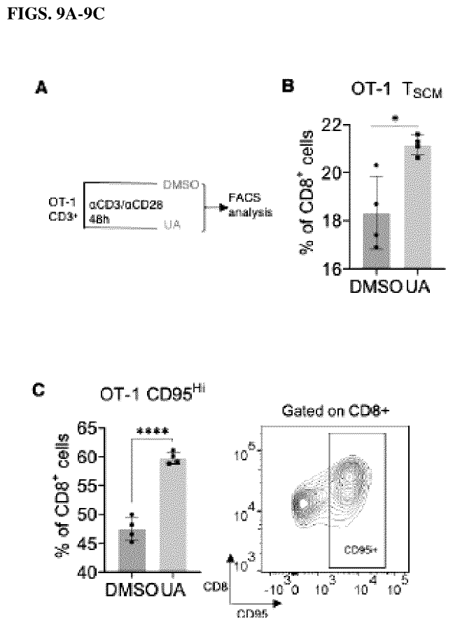

Adoptive cell transfer (ACT) represents the infusion of antigen-specific leukocytes with direct antitumor activity, yet identification, selection and expansion of lymphocyte subsets harboring optimal antitumor qualities remains one of the most crucial challenges (Rosenberg and Restifo, 2015). ACT benefits especially from minimally differentiated cells due to their improved survival and long-term potential to generate unexhausted effector cells (Luca Gattinoni et al; Mo et al., 2021; Roberto et al., 2015). In particular, CD8+ T cells restricted in a stem-like state have been associated with enhanced tumor suppressive properties upon adoptive cell transfer (Enrico Lugli et al; Verma et al., 2021). Having demonstrated that UA induces a T_{SCM} phenotype in murine T cells, we next investigated whether UA could be exploited to improve adoptive immunotherapy. T cells from OT-1 donor mice (FIG. 3A) were cultured for 48 hours in the presence of UA or vehicle control, followed by adoptive transfer into immunodeficient Rag1−/− mice. Prior to ACT, CD8+ T cells from OT-1 donors also displayed a T_{SCM} phenotype (FIG. 9A-B) with enhanced CD95 expression (FIG. 9C). Seven days following the transfer the number of engrafted CD8+ T cells was significantly higher in mice that had received Urolithin A-treated T cells (FIG. 3B) supporting an increased potential to expand. Moreover, when UA-conditioned OT-1 T cells were transplanted into mice bearing palpable ovalbumin-overexpressing APTK (APTK-OVA) tumors (FIG. 3C), this led to a more substantial tumor suppression in comparison to control OT-I T cells (FIG. 3D, E). Analysis of tumors revealed upon UA-T cell transfer a lower expression of CD44 and a higher number of TCF1_{Hi}CD8+ tumor infiltrating T cells (FIG. 3F-G) in tumors in line with maintenance of a UA induced memory phenotype (Schumann et al., 2015; Zhou et al., 2010). CD62L expression remained unchanged (FIG. 3H). Moreover, consistent with an improved memory response, animals receiving UA-pretreated T cells contained less Tim3_{Hi}PD1_{Hi} terminally exhausted CD8+ T cells in the TME (FIG. 3I). Thus, UA augments immune-mediated antitumor memory upon adoptive cell transfer.

Urolithin a Induces Pink1-Dependent Mitophagy in T Cells

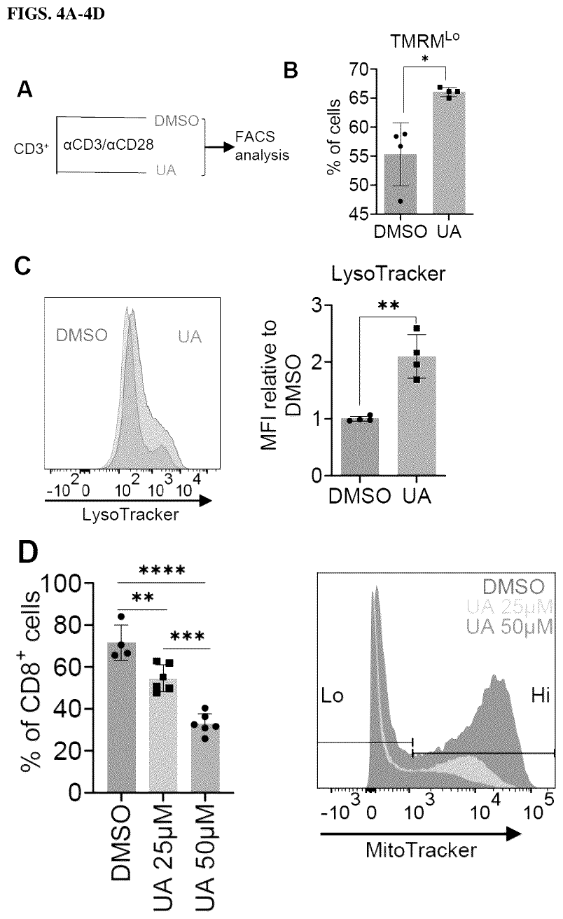

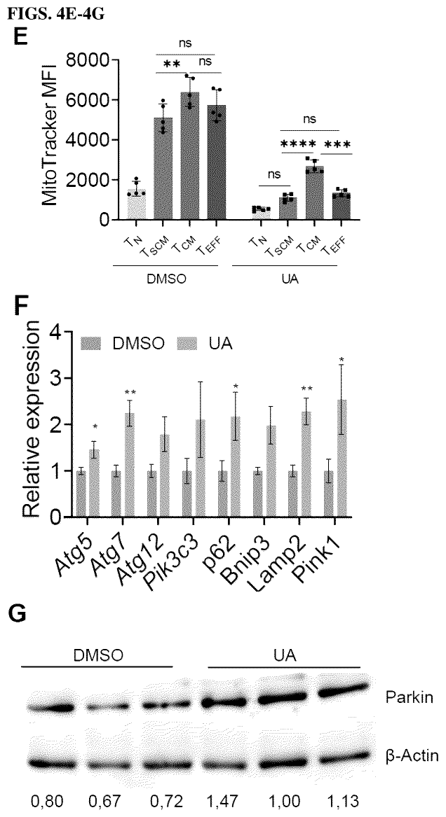

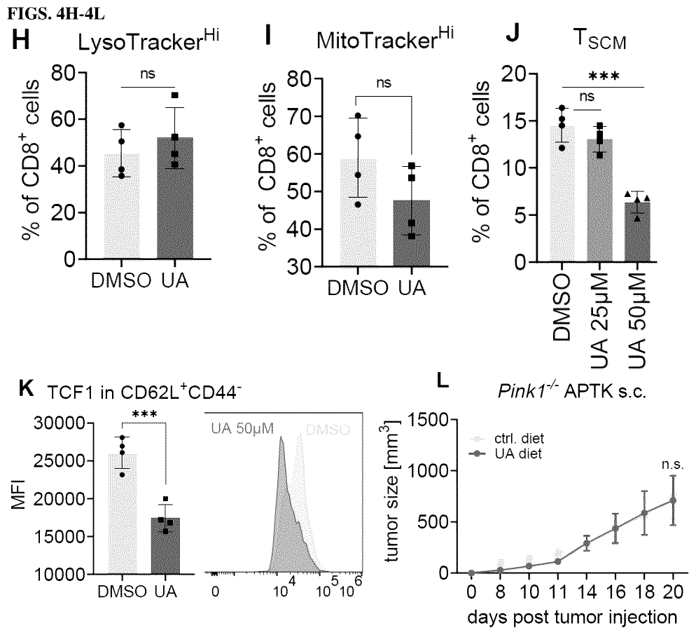

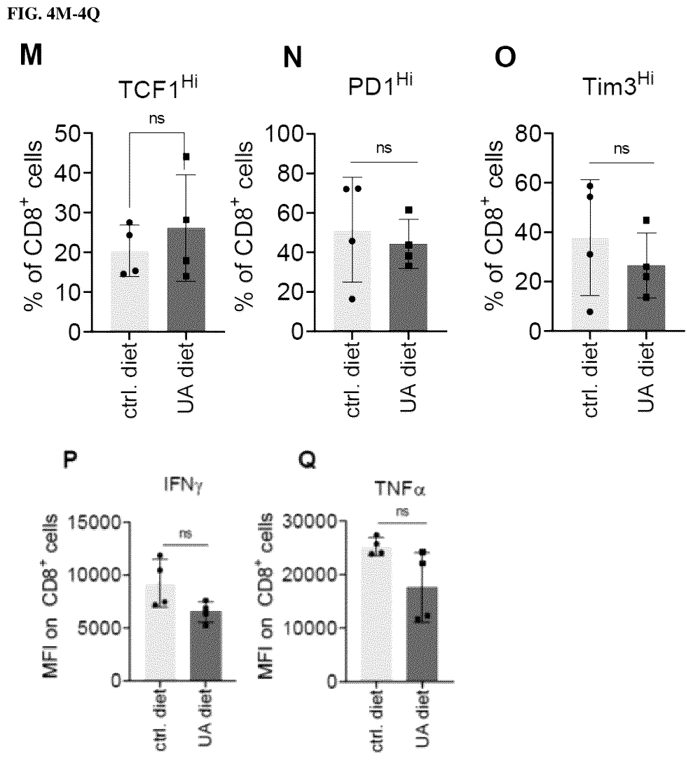

Next it was examined as to whether the shift towards CD44-CD62L+Sca1_{Hi} T_{SCM} cells was triggered by UA-induced mitophagy in T cells. A reduction of mitochondrial membrane potential within six hours after UA administration in CD8+ T cells was confirmed (FIG. 4A-B).

This was accompanied by enhanced lysosome formation (FIG. 4C) and after 24 hours followed by the loss of mitochondrial content in a dose-dependent manner (FIG. 4D). The latter could be detected in all T cell subsets analyzed (T_{SCM}, T_{CM} and T_{EFF}; FIG. 4E), thus suggesting induction of mitophagy.

UA activates mitophagy in myocytes and hippocampal neurons via a Pink1/Parkin mediated stress-response (D'Amico et al., 2021). Also in αCD3/αCD28 stimulated T cells, UA led to a significant upregulation of Atg5, Atg7, p62, Lamp2 and Pink1 gene expression (FIG. 4F). Moreover, UA stabilized Parkin protein expression (FIG. 4G). To functionally confirm the 9 dependence of UA induced T_{SCM} formation on Pink1/Parkin mediated mitophagy, we activated Pink1−/− T cells in the presence or absence of UA. Loss of Pink1 prevented the UA-induced lysosome formation as well as the decrease in mitochondrial content (FIG. 4H, I). Pink1−/− CD8+ T cells failed to exhibit a T_{SCM} phenotype (FIG. 4J), and expressed less TCF-1 in CD44-CD62L+CD8+ T cells (FIG. 4K). Consequently, Pink1 deletion abrogated the tumor suppressive effect of UA (FIG. 4L) while CD8+TIL of Pink1−/− mice did not exhibit differences in TCF-1 expression, exhaustion markers, or ex vivo cytokine release (FIG. 4M-Q) strongly supporting the notion that UA-induced Pink1-dependent mitophagy triggers T_{SCM} formation to enhance antitumor immunity.

UA Induces T_{SCM} Via Cytosolic Release of PGAM5 that Drives Wnt Signalling

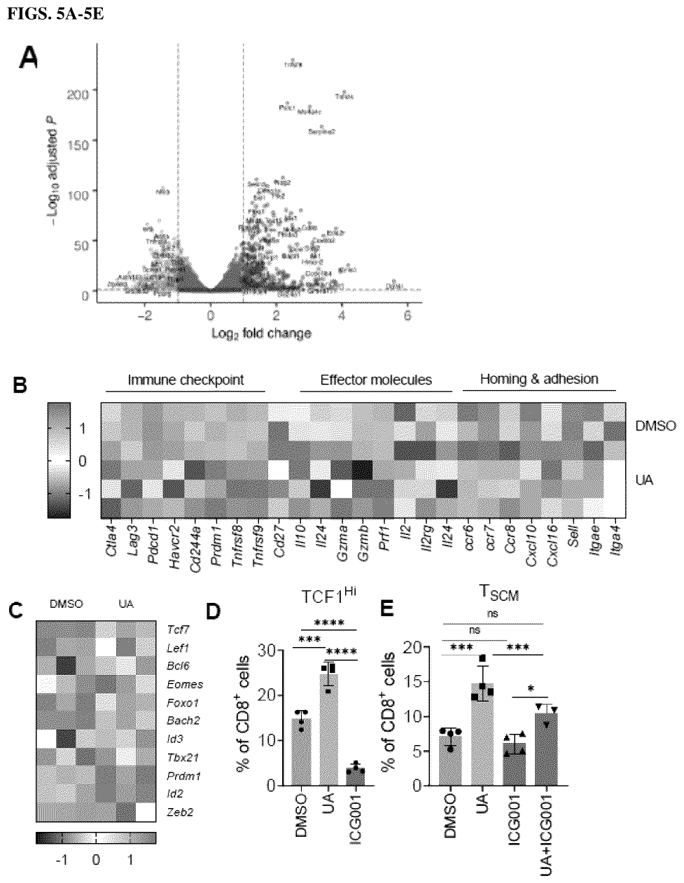

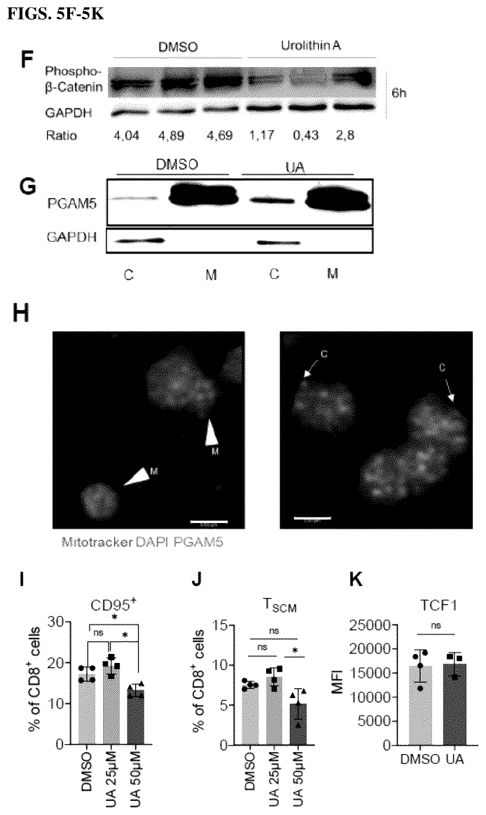

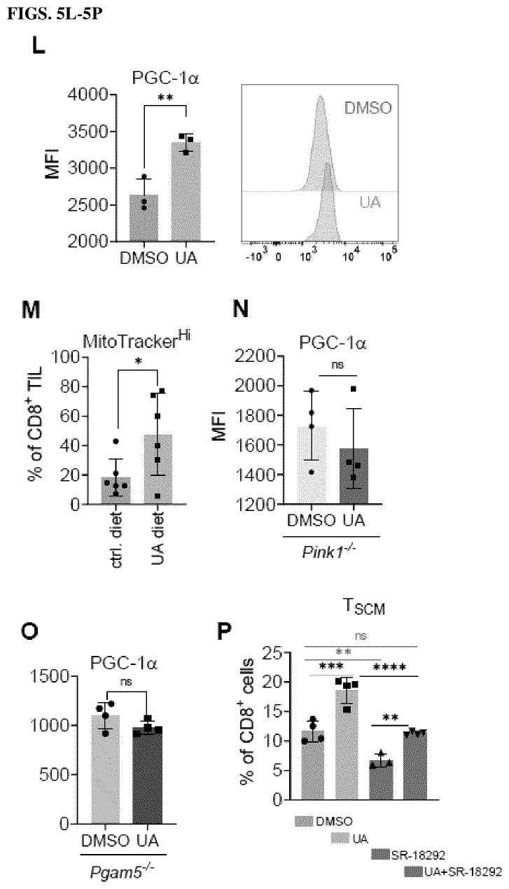

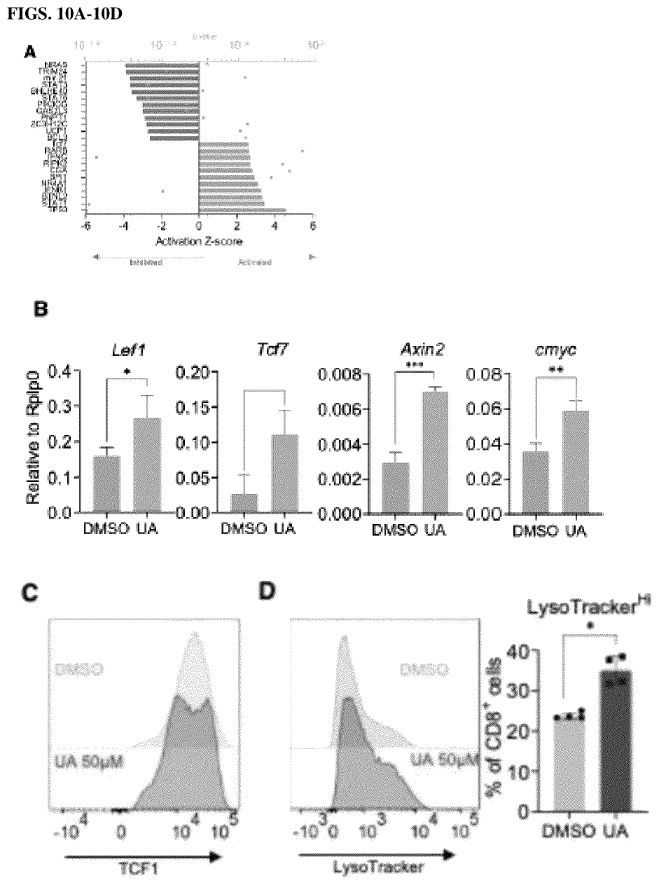

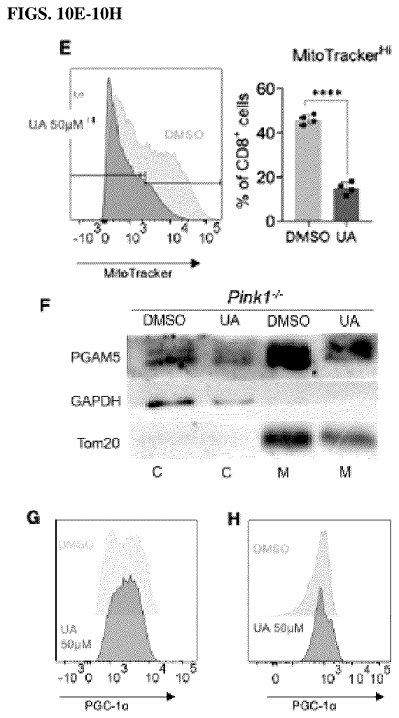

To explore the downstream events linking mitophagy to T_{SCM} formation, we performed RNA sequencing to globally assess differential gene expression in T cells exposed for 48 hours to either DMSO or UA in vitro. A total number of 1178 differentially expressed genes were identified of which 765 were significantly upregulated and 413 downregulated (FIG. 5A). UA treatment reduced the expression of genes that code for immune checkpoints and effector molecules, while enhancing expression of Cd27, Ccr7 and adhesion genes (FIG. 5B), a characteristic of stem-cell like CD8+ T cells (Enrico Lugli et al; Mo et al., 2021; Parisi et al., 2020; Reschke et al., 2021). Additionally, UA-treated cells revealed a special enrichment of genes involved in memory formation such as Tcf7, Bach2 and Bcl6 and reduced expression of effector fate associated genes Prdm1 and Id2 (Ichii et al., 2002; Roychoudhuri et al., 2016; Zhou et al., 2010) (FIG. 5C). In agreement with the increased number of TCF1_{Hi} T_{SCM} cells, upstream regulator analysis of RNAseq data revealed Tcf7 as a possible regulator of UA induced transcriptomic changes on T cells in vitro (FIG. 10A). Indeed, when applying ICG001 to pharmacologically block TCF-1, UA-induced T_{SCM} formation in vitro was abrogated (FIG. 5D-E).

Next it was examined as to whether the observed upregulation of TCF1 was a result of enhanced Wnt signalling. In line with this notion, an increased transcription of several Wnt target genes was observed (FIG. 4B). Moreover, UA led to a marked decrease of β-catenin phosphorylation already after 6 hours (FIG. 5F) indicating activation of Wnt-signaling prior to the transcriptional changes. Mitophagy releases the mitochondrial-bound protein phosphatase phosphoglycerate mutase family member 5 (Pgam5) to the cytoplasm where it has been suggested to block axin-dependent β-catenin degradation thereby inducing mitochondrial biogenesis (Bernkopf et al., 2018; Yamaguchi et al., 2019). To explore whether Pgam5 may be involved in UA-induced Wnt activation, Pgam5 was examined localization upon UA administration. Immunoblot analysis of sub-cellular fractionations revealed that Pgam5 was indeed rapidly released into the cytoplasma upon UA-mediated mitophagy (FIG. 5G), which could also be confirmed by immunofluorescence (FIG. 5H). More importantly, Pgam5 loss blocked the UA-dependent expansion of CD44-CD62L+Sca1_{Hi} T_{SCM} cells (FIG. 5I). UA treated Pgam5−/− CD8+ T cells further failed to upregulate TCF1 or the memory marker CD95 in vitro (FIG. 5J-K, FIG. 10C), despite the fact that UA still enhanced lysosome formation and reduced mitochondrial content (FIG. 10D-E) confirming that cytosolic Pgam5 contributed to UA-induced alterations of T cell function. In line with this notion, lack of cytoplasmic expression of Pgam5 in Pink1−/− CD8+ T cells upon UA exposure was confirmed (FIG. 10F).

Pgam5 dependent Wnt activation has been suggested to trigger compensatory mitochondrial biogenesis in response to mitophagy (Bernkopf et al., 2018). Peroxisome proliferator-activated receptor gamma coactivator 1-alpha (PGC-1α) is considered the master regulator of mitochondrial biogenesis (Finck and Kelly, 2006). Accordingly, a marked upregulation of PGC-1 was detected after prolonged UA administration in vitro (FIG. 5L), which was accompanied by an increased mitochondrial content in TIL of APTK tumors (FIG. 5M), indicating mitochondrial biogenesis and in line with superior T cell fitness in the TME (Dumauthioz et al., 2020; Scharping et al., 2016). UA failed to enhance PGC-1α in Pink1−/− or Pgam5−/− CD8+ T cells (FIG. 5N, 0; FIG. 10G-H), while chemical inhibition of PGC-1α blocked UA-dependent expansion of CD44-CD62L+Sca1_{Hi} T_{SCM} cells (FIG. 5P). Collectively, these results indicated that UA drives T_{SCM} formation via a Wnt-dependent upregulation of PGC-1α, promoted by the cytosolic release of Pgam5 in response to mitophagy.

UA Promotes Human T_{SCM}, Facilitating Generation of Potent CAR T_{SCM}

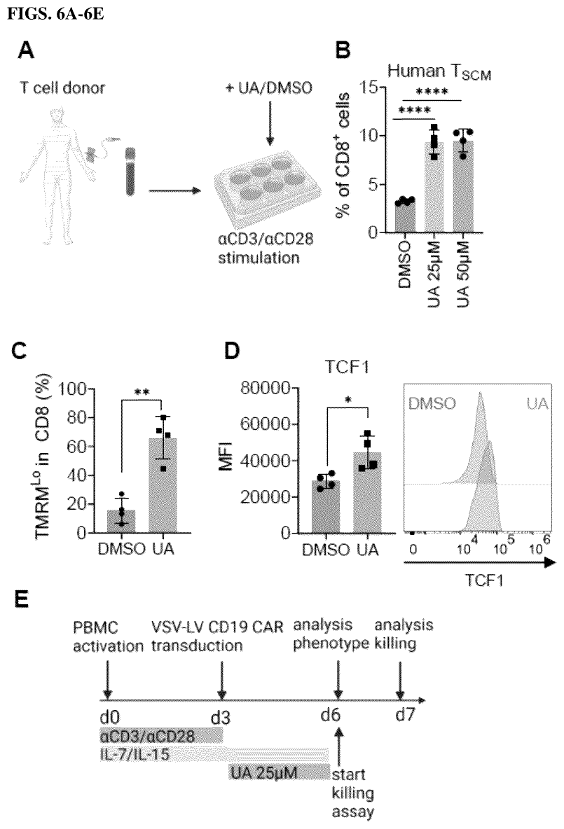

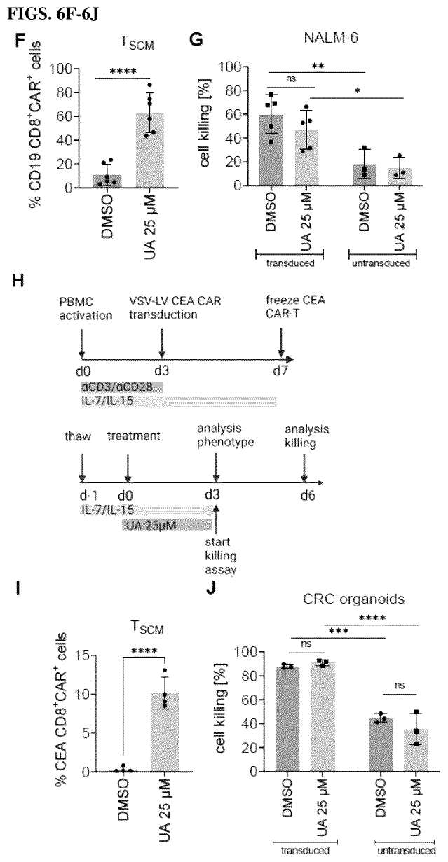

Finally, an examination as to if UA causes expansion of T_{SCM} cells in human CD8+ T cells was conducted. Human CD3+ T cells were isolated from PBMCs of healthy donors and stimulated them with αCD3/αCD28 beads in vitro in the presence of UA (FIG. 6A). Indeed, UA increased the frequency of human T_{SCM} cells based on the expression of CD45RA+CCR7_{Hi}CD62L+CD95+CD8+ in five out of five individual donors (FIG. 6B, FIG. 11A, B). Like in murine T cells, after 48 hours UA-treated human CD8+ T cells displayed a decreased mitochondrial membrane potential (FIG. 6C) and intracellular staining confirmed increased TCF1 expression (FIG. 6D) validating that UA induces memory stem cell features in both murine and human T cells.

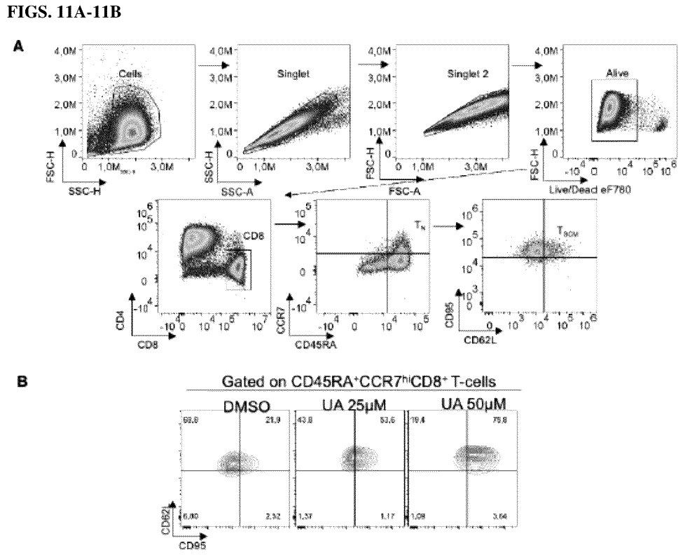

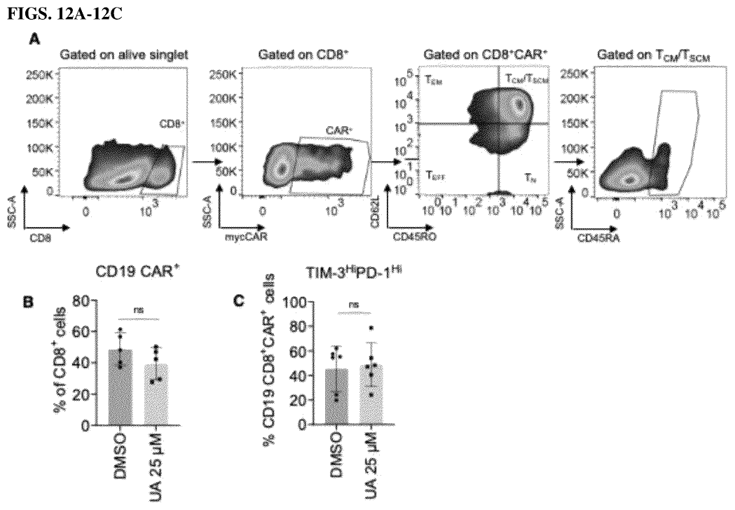

Infusion of T cells carrying an engineered chimeric antigen receptor (CAR), designed to specifically guide leukocytes to recognize and eliminate malignancies of interest, has shown impressive clinical results, but incomplete remissions and frequent relapses after successful therapy highlight the need to improve sustained antitumor response (June et al., 2018; Majzner and Mackall, 2019). Recent data suggests that CAR-T cells containing high fractions of T_{N/SCM} show improved tumor killing and the exclusive ability to counteract leukemia re-challenge in hematopoietic stem/precursor cell-humanized mice (Arcangeli et al., 2022). To determine whether UA-triggered mitophagy also constitutes a feasible strategy to induce CAR-T_{SCM}, activated T cells were transduced with the CD19-CAR gene by a lentiviral vector (VSV-LV) in the presence or absence of UA. Three days post transduction, the amount of CAR-expressing T_{SCM} was determined (FIG. 6E; FIG. 12A). UA did not impair gene delivery into CD8+ cells (FIG. 12B), yet while CAR expressing T_{SCM} cells were markedly increased and comprised about 60% of CD8+ cells upon UA exposure (FIG. 6F), this did not have a negative impact on CD19 CAR-T cell mediated killing of NALM-6 leukemia cells (FIG. 6G). Markers of exhaustion were not affected by UA supplementation (FIG. 12C).

Even when UA was applied to previously frozen CAR-T cells specific for carcinoembryonic antigen (CEA; FIG. 6H), this strongly enhanced CAR T_{SCM} formation (FIG. 6I) with comparable killing efficacy of CEA-expressing human CRC organoids (FIG. 6J). Thus, UA markedly enhances expansion of CAR-T_{SCM} cells.

### Example 4: In Vivo Model of Heart Failure

Generation of a rat model of cardiac impairment following myocardial infarction Fifty (50) Wistar male rats from Janvier Labs (France), 6-week-old (200-224 g on the day of arrival), were included in this study. Acclimatization of animals lasted at least 5 days. During the study, animals had free access to food and drinking water ad libitum.

Myocardial infarction (MI) was induced by chronic left anterior descending coronary artery (LAD) ligation performed at DO. Rats were anesthetized with an intraperitoneal (IP) injection of Medetomidine (0.5 mg/kg) and Ketamine (50 mg/kg), intubated and ventilated at 10 mL/kg tidal volume and 70-80 cycles/min. Body temperature was maintained between 36.5° C. and 37.5° C. via a thermoregulated heating pad connected to a rectal probe. A left lateral thoracotomy was carefully performed so as to expose the heart. Then, a suture was placed around the LAD (4/0 Silk, Ethicon), around 2 mm below the left atrium and close to the interventricular junction, in order to obtain infarct size (IS) near 40% of the total left ventricular (LV) area. Chest was closed, air was expelled from the rib cage to avoid pneumothorax and quick reanimation was performed using atipamezole hydrochloride (IM, 0.5 mg/mL, Zoetis). A peri-operative care of the animals was performed during the surgery:
Pre-operative care by one carprofen injection (SC, 5 mg/kg, Pfizer) and one lidocaine injection on the site of incision (SC, 4 mg/kg, Vetoquinol).
Post-operative care: 3 injections of buprenorphine (SC, 0.01 mg/kg, Axience) administered after awakening and during the first 24 hours after the surgery.

Sham animals from were subjected to the same protocol than MI groups. However, after the left lateral thoracotomy exposing the heart, the rib cage was closed without passing the suture thread around LAD.

Echocardiography

Rats underwent transthoracic echocardiographic examination in order to assess cardiac morphology and function in a noninvasively way. Echocardiography was performed by using a digital ultrasound system (Vivid 7, GE Medical Systems) equipped with a 10 MHz phased array and a 13 MHz linear-array transducer. All images acquisitions, measurements and calculations were done in accordance to the American Society of Echocardiography (Schiller, N. B. et al. 1989) and previous validation of the method in rat (Litwin et al. 1994). Rats were anesthetized with 4% isoflurane in 50% oxygen-50% air mixture and maintained with 2-2.5% isoflurane during the procedure. They were placed in supine position on a heating pad and thorax-shaved.

Body temperature was monitored and maintained between 36.5-37.5° C. via a rectal probe connected to a thermoregulated heating pad. Standard B-mode (Brightness-mode) and M-mode (Motion-mode) images of the heart were obtained in the two-dimensional (2D) parasternal long axis view (PSLA). LV parameters were measured and calculated as the mean of 3 consecutive cardiac cycles by a single blinded trained operator.

Left ventricle (LV) function was assessed using ejection fraction (EF), calculated as follow:

EF=ejection fraction=[(End diastolic volume−End systolic volume)/End diastolic volume]*100.

FS=fractional shortening=[(LVIDd−LVIDs)/LVIDd]*100.

Other parameters were measured for animal randomization, and for the calculation of FS: LVIDd and LVIDs=LV internal diameter in diastole and systole.

A total of two (2) echocardiographic exams were performed for all the animals. The first examination took place 5 days after surgery. It was used as a control of the surgery to exclude rats with small infarcts and limited reduction of EF. Animals were randomized in three homogeneous groups based on LVIDd and EF. After the randomization, a longitudinal follow-up study by echocardiography was performed on the 3 groups of rats. The 2nd echocardiographic examinations were done at Month 2 (M2) following the start of the treatments. They were used to assess the cardiac remodeling and function following MI, based on the echocardiographic parameters previously described.

Treatment of the Animals

Rats were administered by oral gavage from day 5 post-surgery up to 2 months following the start of the treatments. Groups were split as follow:
Group 1: Sham surgery, vehicle carboxylethylcellulose (CMC) 0.5%, n=10
Group 2: MI, CMC 0.5%, n=20
Group 3: MI, 66 0.5 mpk, n=20

Statistics

Statistical analysis was performed with the Graphpad 6 software. If values were normally distributed, a parametric analysis was performed. First, differences were assessed between Sham/Vehicle and MI/Vehicle using an unpaired ttest followed by the appropriate post-hoc test. Then, the MI/66 group was compared to MI/Vehicle using an unpaired t-test followed by the appropriate post-hoc test.

Results

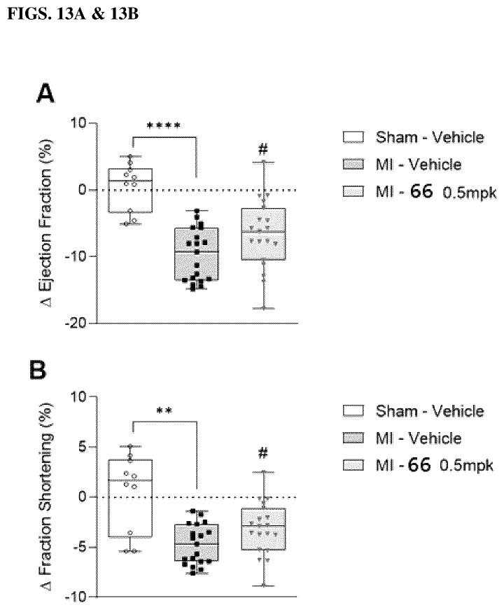

Progression of left ventricle (LV) dysfunction induced by myocardial infarct caused a significant reduction of EF and FS (p<0.0001 & p<0.01, MI/Vehicle vs Sham/Vehicle group; FIG. 13). Compound 66 prevented the LV dysfunction MI-induced, with significant increases of EF and FS parameter in M-mode (p<0.05 and p<0.05, MI/66 vs MI/Vehicle; FIG. 13.

In conclusion, compound 66 (0.5 mg/kg) was able to prevent LV systolic dysfunction induced by MI 2 months after LAD ligation, with a significant improvement of systolic function (EF & FS).

### Example 5: Effects of Test Compounds on Mitochondrial Content in Human T Lymphocytes

Human peripheral blood mononuclear cells were purified with a Ficoll-Paque (GE healthcare) gradient. T cells were enriched with EasySep Human T Cell Isolation Kit (StemCell) and activated with dynabead Human T-Activator CD3/CD28 for T Cell Expansion and Activation Kit (11131D; ThermoFisher Scientific). Cells were activated with the compound, i.e. compound 66, 77, and 77A, at the indicated concentration or DMSO. Mitochondria were stained with Mitotracker Red for 45 min before performing the quantification of Mitotracker Mean Fluorescent Intensity (MFI) by FACS.

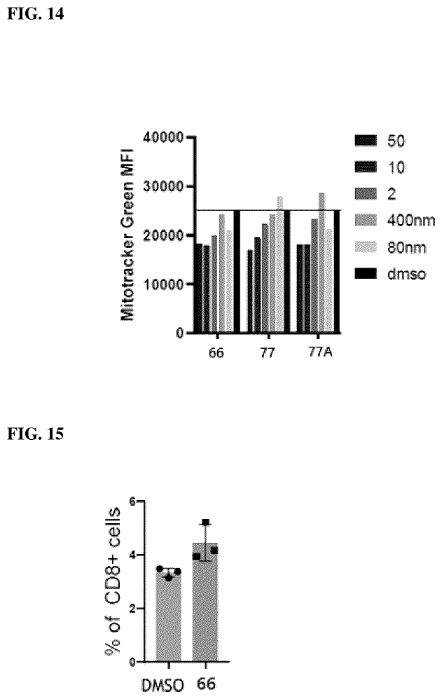

Results shown in FIG. 14 showed that there is a dose dependent decrease in mitochondrial content in Lymphocytes T cells incubated with the compound, which corresponds to an activation of the mitophagy pathway.

### Example 6: Effects of Test Compounds on the Proportion of Human T Memory Stem Cells (Tscm)

Human peripheral blood mononuclear cells were purified with a Ficoll-Paque (GE healthcare) gradient. T cells were enriched with EasySep Human T Cell Isolation Kit (StemCell) and activated with dynabead Human T-Activator CD3/CD28 for T Cell Expansion and Activation Kit (11131D; ThermoFisher Scientific). Cells were activated with compound 66 at the indicated concentration or DMSO. Tscm cells are identified and counted based on the expression of five different markers: CD45RA⁺CCR7^{Hi}CD62L⁺CD95⁺CD8⁺.

Results shown in FIG. 15 demonstrated that there is a significant increase in Tscm proportion of CD3+ lymphocytes following stimulation with compound A. This increase in the percentage of Tscm is known to be associated with an improved response to immunotherapy and can be exploited for CAR-T cell generation.

### Example 7: Compounds Reduce Tumor Growth and Sensitize Tumors to PD-1 Blockade

In one example, C57BL/6J mice are subcutaneously transplanted with APTK organoids, characterized by loss of Apc, Trp53 and Tgfbr2 as well as expression of oncogenic Kras^{G12D} (Nicolas et al., Cancer cell 2022, 40, 168-184.e13.; Varga et al., The Journal of experimental medicine, 2020; 217). APTK organoids are mechanistically disrupted, incubated with Accutase to generate a single cell suspension and reconstituted in PBS 20% Matrigel (Corning) for subcutaneous transplantation.

Ten days after organoid transplantation, mice are then administered with a compound of the present invention, e.g. 66, 77, or 77A, or vehicle orally either by gavage or by food admix for up to 20 days. Tumor sizes are measured at several timepoints after transplantation and tumor volume quantified as ½(width²×length). Results show that the administration of the compound lead to a significant decrease in tumor size, compared to vehicle treated animals.

In another experiment, C57BL/6J are first administered orally with compound or vehicle, either by gavage or by food admix, one week before subcutaneous transplantation with APTK organoids. Oral treatment with compound is continued for up to 20 days post transplantation. To study the combination efficacy with immunotherapy, mice are injected i.p. with 100 μl nVivoMAb anti-mouse PD-1 (Hoelzel; BE0146) and InVivoMab mouse IgG2b isotype control (Hoelzel; BE0086) every 2 days, starting three days after tumor injection. Tumor sizes are measured at several timepoints after transplantation and tumor volume quantified as ½(width²×length).

Results show that compound treatment sensitize APTK tumors to PD-1 blockade, while checkpoint inhibition alone does not affect APTK-tumor growth.

### Example 8: Compounds Improves Tumor Therapy by Adoptive T Cell Transfer

In one example, splenic T cells are isolated from OT-1 donor mice and activated using αCD3/αCD28 stimulation beads for 48 h in presence of with a compound of the present invention, e.g. 66, 77, or 77A, or vehicle control. After magnetic bead removal, activated T cell are intravenously injected into Rag1−/− recipients mice. Seven days following the transfer, spleens are collected in order to isolate splenic CD8+ T cells and count them. Results show that the number of engrafted CD8+ T cells is significantly higher when cells are pre-treated with compound before cell transfer, supporting an increased potential to expand.

In another experiment, compound-conditioned OT-1 T cells are transplanted into mice bearing palpable ovalbumin-overexpressing APTK (APTK-OVA) tumors, 10 days after subcutaneous transplantation of APTK-OVA cells. Tumor sizes are measured at several timepoints after transplantation and tumor volume quantified as ½(width²×length). Results demonstrate that tumor size is smaller in mice that received compound-conditioned OT-1 T cells, meaning that these cells have a stronger tumor suppression capacity, compared to control OT-I T cells.

At the end of the in vivo experiment, tumors are collected to isolate activated T cells. Quantification of the proportion of CD8+ cells also expressing high levels of Tim3 and PD1 show that animals receiving compound-pretreated T cells contained less Tim3^{Hi}PD1^{Hi} terminally exhausted CD8+ T cells in the tumor microenvironment. Thus, the compound augments immune-mediated antitumor memory upon adoptive cell transfer.

### Example 9: Compound Facilitate the Generation of Potent CAR Tscm Cells

In one experiment, human activated T cells are transduced with the CD19-CAR gene by a lentiviral vector (VSV-LV) in the presence or absence of with a compound of the present invention, e.g. 66, 77, or 77A. Three days post transduction, the amount of CAR-expressing TSCM is determined. Tscm cells are identified and counted based on the expression of five different markers: CD45RA+CCR7^{Hi}CD62L⁺CD95⁺CD8⁺.

Results show that CAR expressing T_{SCM} cells are markedly increased upon treatment with compound. Thus, the compound markedly enhances expansion of CAR-T_{SCM} cells.

### Discussion

UA has been suggested as an encouraging nutrient addition to target age-related conditions (D'Amico et al., 2021). In various preclinical models UA has proven effective to attenuate inflammation as well as to improve muscle, brain and joint functions. In addition to this wide spectrum of potential applications covering the fields of neuro-degeneration, muscle disorders and inflammatory diseases, we demonstrate here that UA induces a protective anti-tumor CD8+ T cell immunity strongly supporting its benefit for cancer therapy either in combination with immune checkpoint blockade or in the context of adoptive T cell therapy. The data disclosed herein suggests that mitophagy induction represents an attractive option for therapy of established tumors as it promotes an adaptive T cell response via a dual mechanism: enhancing antigen presentation in tumor epithelia as well as directly affecting T cell fate.

T_{SCM} are thought to enhance immunity by constantly giving rise to less exhausted effector T cells in the TME (Gattinoni et al., 2017). Multiple pathways are expected to govern T cell fate decisions, balancing between stemness and terminal differentiation (Luca Gattinoni et al; Pilipow et al., 2018; Scholz et al., 2016). We proposed a complex interplay linking intrinsic mitochondrial cues and subsequent activation of the stemness promoting Wnt-driven Tcf7 transcription. The effect of UA on T_{SCM} formation appears to be both dependent on Pink1 and Pgam5. Interestingly, Pgam5 has been suggested to regulate Pink1/Parkin mediated mitophagy (Yu et al., 2020a), yet we observed adequate induction of mitophagy in Pgam5−/− CD8+ T cells. Of note, it was also observed that compensatory mitochondrial biogenesis second to mitophagy drives T cell stemness decisions. Wnt-β-Catenin signalling has been suggested to induce mitochondrial biogenesis (Bernkopf et al., 2018) and we propose PGC-1α as one critical regulator that is directly activated by Wnt-signalling and essential to T_{SCM} formation as it has recently been proposed in the context of Mek inhibition (Verma et al., 2021). Similarly, we also observed a downregulation of cyclin D1 leading to an arrest of proliferation and effector differentiation (Gattinoni et al., 2009; Pilipow et al., 2018; Verma et al., 2021).

The concept of pharmacologically altering mitochondrial dynamics has recently been proven to be effective. Increased mitochondrial clearance via nicotinamide riboside (NR) could reinforce stem-cell fate decisions in hematopoetic stem cells (HSCs) (Vannini et al., 2019) and improved responsiveness to anti-PD1 treatment (Yu et al., 2020b). Our data highlight the requirement of mitophagy for TSCM formation and describe the downstream signaling pathway linking mitochondrial dynamics and adaptive immunity. Furthermore, a potential application of UA was identified due to its capacity to expand CAR TSCM. CAR TSCM cells confer an improved and sustained anti-tumor efficacy upon repeated exposure to tumor cells in vivo (Arcangeli et al., 2022). Furthermore, CAR-TSCM are intrinsically less prone to induce severe cytokine release syndrome (Arcangeli et al., 2022). Such subsets are currently selected via cell sorting, thus constituting a costly and time-consuming manufacturing step (Abou-El-Enein et al., 2021). By the addition of UA after CAR gene transduction, it suggests a readily-applicable, scalable method of rapidly generating potent CAR-TSCM that avoids premature activation and exhaustion, bypassing manufacturing challenges currently presenting an unmet need in CART cell therapy.

Previous studies have suggested in vitro antiproliferative and apoptotic activity of both pomegranate juice and purified elagatinnins in CRC cell lines (Jaganathan et al., 2014; Seeram et al., 2005). Considering the particular limitations of ellagitannin metabolization into the bioavailable derivate UA, which shows large inter-individual variations due to the requirement of an adequate gut microbiome (Cortds-Martin et al., 2018), clinical applications of pomegranate juice and ellagitannins may be limited. Of note, concentrated forms of UA have been associated with tumor-limiting properties both in pancreatic cancer cells in vitro and in murine PDAC upon oral gavaging, attributed to the suppression of the PI3K/AKT/mTOR pathway and alterations in the TME (Totiger et al., 2019). It has been demonstrated here that the effect of UA appears to be primarily dependent on CD8+ T cells. This could be in part explained by a context specific sensitivity to mTOR inhibition as our colorectal tumor organoids are only partially responsive to mTOR inhibition (Greten, unpublished observations). Nevertheless, the clear shift towards TSCM cells suggests that UA-dependent anti-tumor immunity may not be limited to colorectal cancer. Yet, the complex inflammatory TME of the colon is characterized by a diverse interplay between tumor cells, immune and stromal cells, as well as microbiome that governs carcinogenesis (Schmitt and Greten, 2021). Considering the broad effects of UA and its systemic nature (D'Amico et al., 2021), and in vitro evidence of UA affecting macrophage polarization (Boakye et al., 2018), we cannot exclude that other components that constitute the TME apart from CD8+ T cells and antigen-expressing tumor cells may also be affected by UA in a biologically-relevant manner.

Collectively, the data disclosed herein suggests that the orally-available and well tolerated mitophagy inducer UA confers CD8+ T cell dependent anti-tumor effects in CRC. The fact that UA induces a T_{SCM}-associated phenotype in human leukocytes and CAR T cells ex vivo indicates that these findings can be translated into human disease and provide a solid rationale for future clinical trials to examine whether oral UA supplementation or in vitro treatment of leukocytes can be used for more effective tumor therapy.

Additionally, the activity of UA disclosed herein can be extended to the UA derivatives disclosed herein, e.g. 66, 77, and 77A. Accordingly, the UA derivatives disclosed herein, e.g. 66, 77, and 77A, confer CD8+ T cell dependent anti-tumor effects, and are useful in treating cancer and/or enhancing cancer immunotherapy.

### Example 10: Effects of Compounds in a Rat Primary Culture of Cortical Neurons and Microglial Cells Injured with Aβ1-42

Primary Coculture of Cortical Neurons and Microglial Cells

Pregnant female rat (Wistar) of 15 days of gestation were killed using a deep anesthesia with CO₂ chamber and a cervical dislocation. Fetuses were collected and immediately placed in ice-cold L15 Leibovitz medium with a 2% penicillin (10,000 U/mL) and streptomycin (10 mg/mL) solution (PS) and 1% bovine serum albumin (BSA). Cortex were specifically dissected and then were treated for 20 min at 37° C. with a trypsin-EDTA solution at a final concentration of 0.05% trypsin and 0.02% EDTA. The dissociation was stopped by addition of Dulbecco's modified Eagle's medium (DMEM) with 4.5 g/L of glucose, containing DNAse I grade II (final concentration 0.5 mg/mL) and 10% fetal calf serum (FCS). Cells were mechanically dissociated by three forced passages through the tip of a 10-mL pipette. Cells were then centrifuged at 515×g for 10 min at 4° C. The supernatant was discarded, and the pellet was resuspended in a defined culture medium consisting of Neurobasal medium with a 2% solution of B27 supplement, 2 mmol/liter of L-glutamine, 2% of PS solution, 10 ng/mL of brain-derived neurotrophic factor (BDNF), 2% of heat-inactivated horse serum, 2% of heat-inactivated FCS, 1 g/L of glucose, 1 mM of sodium pyruvate, and 100 μM of non-essential amino acids. The presence of serum in the medium allows the growth of the microglial cells. Viable cells were counted in a Neubauer cytometer, using the trypan blue exclusion test.

The cells were seeded at a density of 45 000 per well in 96-well plate precoated with poly-L-lysine and were cultured at 37° C. in an air (95%)—CO₂ (5%) incubator. The medium was changed every 2 days.

Test Compound and Aβ1-42 Application

On day 11 of culture, the compounds were dissolved in the appropriate vehicle and pre-incubated with the primary cortical neurons for one hour (1 h), before Aβ1-42 exposure.

On day 11 of culture, the cortical neurons were injured with the AP 1-42 peptide. AP 1-42 peptide was dissolved in the defined culture medium mentioned above, at an initial concentration of 20 μM. The solution was gently agitated for 3 days at 37° C. in the dark and immediately used after being properly diluted in culture medium to the concentration used (5 μM, 0.5 μM oligomers precisely evaluated by automatic WB). The AP 1-42 solution was applied on the culture at 5 M (0.5 μM oligomers, AβO), in presence of the compound, for 72 hours.

Immunostaining: MAP-2 and OX-41

72 hours after the application of Aβ, the cell culture supernatant was removed. Cells were then washed with phosphate-buffered saline (PBS) and fixed with a cold solution of ethanol (95%) and acetic acid (5%) for 5 min at −20° C. After two washing steps with cold PBS, cells permeabilized and unspecific binding sites were blocked with a solution of PBS containing 0.1% of saponin and 1% of FCS, for 15 min at RT. Cells were incubated for 2 hours with:
a) Monoclonal anti-MAP2 antibody produced in mouse at dilution of 1/400 in PBS containing 1% FCS, 0.1% saponin, for 2 hours at room temperature.
b) Rabbit polyclonal antibody anti-SIRP alpha/CD172a (OX-41) at dilution of 1/400 in PBS containing 1% fetal calf serum and 0.1% of saponin (this antibody stains specifically the microglia and will allow to evaluate the activation of the microglial cells). These antibodies will be revealed with Alexa Fluor 488 goat anti-mouse IgG at the dilution 1/800 and with Alexa Fluor 568 goat anti-rabbit IgG at the dilution 1/400 in PBS containing 1% FCS, 0.1% saponin, for 1 h at room temperature.
c) Cell nuclei were counterstaining with Hoechst dye (1/1000, sigma).

Primary antibodies were revealed with anti-rabbit IgG and with anti-chicken IgG in PBS containing 1% FCS, 0.1% saponin, for 1 hour at room temperature. Nuclei were labeled with Hoechst dye.

Automatic Computer Analysis

For each condition, 30 pictures (representative of the whole well area) were automatically taken using ImageXpress® (Molecular Devices) at 20× magnification using the same acquisition parameters. From images, the analyses were automatically performed by MetaXpress® (Molecular Devices). The following read-outs were measured:
Total neuron survival (number of MAP-2(+) neurons),
Total length of neurite network of MAP-2(+) neurons (in μm)
Total area of activated microglia (area of OX-41 signal, in μm²)

Statistics

All values are expressed as mean+/−SEM (standard error of the mean). Statistical analysis was performed by one-way ANOVA followed by Fisher's LSD test, with GraphPad Prism. p<0.05 was considered significant.

Results

33 was able to significantly protect the injured neurons submitted to Aβ1-42 (survival and neurite network), at the concentrations of 123 and 370 nM (FIG. 16A-B). In addition, a significant decrease of the microglial activation was observed between 41.1 nM and 370 nM (FIG. 16C).

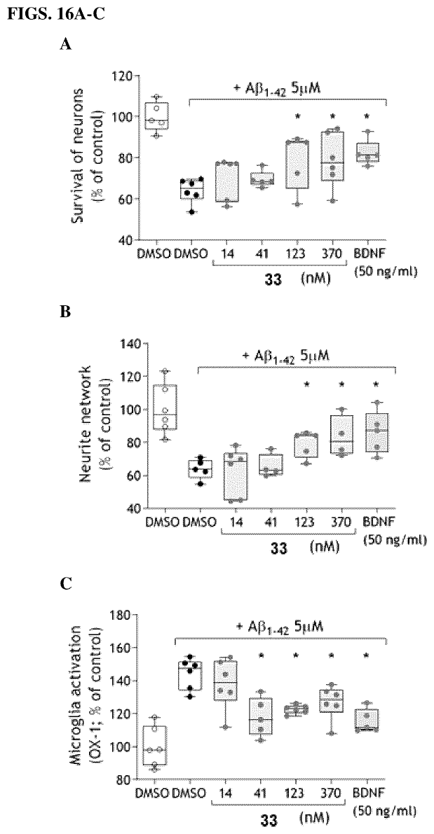

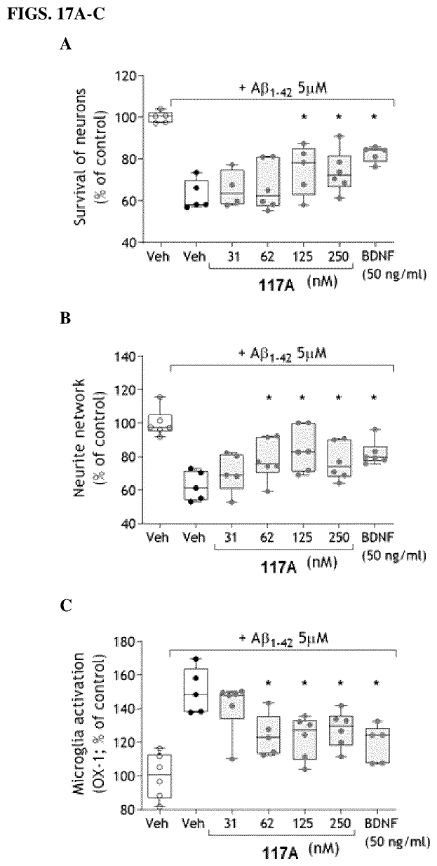

117A was able to significantly protect the injured neurons submitted to Aβ1-42 (survival and neurite network) and to decrease of the microglial activation at the concentrations of 125 and 250 nM (FIG. 17).

The results indicate that 33 and 117A are neuroprotective in this inflammatory in vitro model. In addition, the compounds were able to significantly reduce the microglial activation.

### Example 11: Effects of a Compound in an In Vivo Model of Alzheimer's Disease

Generation of a Mouse Model of Alzheimer's Disease, Based on Intrahippocampal Injection of Human Amyloid Beta 1-42 Oligomer Preparation.

A total of 18-month-old C57BL6 male mice were included in the study (n=12 per group). The animals were housed up to 4 to a cage, in clear polycarbonate cages with a stainless steel mesh lid and floor in a day/night inversed cycle (12 h/12 h). Drinking water and food were supplied ad libitum to each cage. The animals were kept for a period of acclimation of a minimum of 5 days before any handling.

All mice were subjected to surgery and were injected with 2 μL of vehicle or of A 1-42 solution. Aβ1-42 peptide (Bachem) was dissolved in the vehicle, at an initial concentration of 100 μM (the mother preparation was characterized by automatic WB, and precisely contained 15% of AβO). This solution was gently agitated for 3 days at 37° C. in the dark before any use until the day of the surgery.

The day of the surgery, mice were anesthetized by isoflurane (4%, for induction), in an induction chamber coupled with a vaporizer and to an oxygen concentrator. Mice were placed on the stereotaxic frame. Anesthesia was maintained by isoflurane (2%) with a face mask coupled to the isoflurane vaporizer and oxygen concentrator machine. Skull was exposed, and holes were drilled.

The Aβ1-42 preparation was bilaterally injected into the striatum oriens, striatum pyramidal and striatum radiatum of the CA1 area of the hippocampus (at three different depths). A total of 2 μL/side of A 1-42 preparation (100 μM), or vehicle, were bilaterally injected with a Hamilton syringe in the CA1 area of the hippocampus (0.2 μL/min with an Elite Nanomite syringe pump).

Depth of anesthesia and rectal temperature were verified every 5 minutes. After surgery, mice were allowed to recover before going back in their cages.

Oral Treatment of the Animals

Vehicle (0.5% carboxy methyl cellulose (CMC)) or 33 at 1 mg/kg/d was administrated daily by gavage (per os: p.o). The treatment started 1 day after the surgery (D+1) and lasted until the end of the study (D+27). The study was stopped 4 weeks after the surgery (on D28).

Evaluation of Short-Term Spatial Memory—Y-Maze.

The week before the surgery, a training session was performed, for habituation (5 minutes) to the maze. Fourteen (14) days after the surgery, mice were tested for short-term memory deficit in the Y-maze test.

The test was based on two trials:
Trial 1. Mice were allowed to freely explore two arms of the Y-maze during 5 min. The third arm remained closed during the trial. At the end of the 5 min, the animal was placed for 3 min in an empty cage. The Y-maze was cleaned with acetic acid to neutralize odors.
Trial 2. Mice were then allowed to freely explore the three arms of the Y-maze during 5 min. This trial corresponds to the test session.

Both trials were recorded by a video camera using Ethovision system (Noldus). The following items were automatically recorded: a) total distance in the Y-maze and b) the time spent in each arm.

Tissue Collection for Immunostaining

At the end of the experiment (on day 28), 5 mice per group were deeply anesthetized and plasma samples were collected. Mice were then perfused with cold PBS (3 minutes), and cold paraformaldehyde (PFA) 4% in PBS (for 3 minutes).

Brains were dissected and further fixed in PFA 4%, for 2 h at room temperature. After 2 hours, the brains were placed in 30% sucrose in Tris-phosphate saline (TBS) solution overnight at 4° C. The cutting was performed the following day on the whole area of interest.

Serial coronal sections, including the hippocampus area, of 40 μm-thickness were cut using a cryostat (Epredia, Cryostar NX50).

For immunostaining, sections were incubated in TBS with 0.25% bovine serum albumin, 0.3% Triton X-100 and 1% goat serum, for 1 hour at room temperature. This incubation blocked unspecific binding sites and permeabilized the tissues.

Histology

Six (n=6) brain sections per animal were processed and incubated for 24 hours at 4° C. or 2 hours at room temperature with selected antibodies (Abs). The following antibodies were used:
NeuN: Rabbit polyclonal antibody anti-NeuN (1/1000).
Iba1: Rabbit polyclonal antibody anti-Iba1 (1/500).
Tau phospho Ser212/Thr214 (AT100): Mouse monoclonal antibody anti-AT100 (1/200).

These antibodies were revealed with Alexa Fluor 488 anti-rabbit IgG, Alexa Fluor 568 anti-chicken IgG and Alexa Fluor 647 anti-rabbit IgG, at the dilution 1/500, incubated in TBS with 0.25% donkey serum albumin, 0.3% Triton X-100 and 1% goat serum.

Images were acquired with a confocal microscope LSM 900 with Zen software at 20× magnification (Zeiss) using the same acquisition parameters (automatic acquisition). From images, the analyses were automatically performed by MetaXpress® (Molecular Devices). The following read-outs were measured:
Number of NeuN positive pyramidal cells in the CA1 area.
Activation of Iba1-positive microglial cells in the CA1 area.
Activation of AT100 positive neurons in the CA1 area.

Statistics

Results were analyzed using GraphPad Prism software version 8.0.2. *p<0.05 after a One-way or Two-way ANOVA followed by Fisher's test was considered significant.

Results

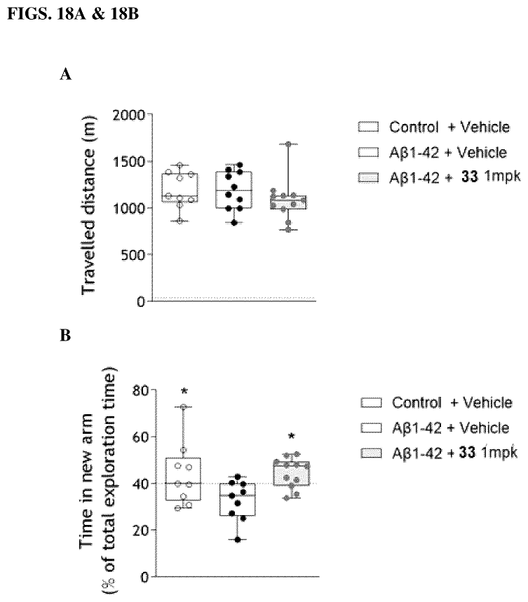

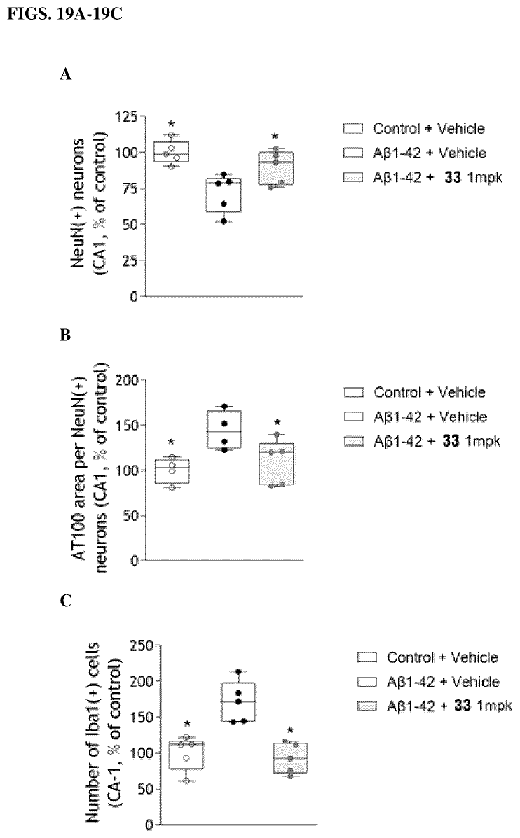

The forced alternation Y-maze test relies in the natural tendency of rodents to explore the new environments and was used to evaluate short-term spatial memory and exploratory behavior of injured mice. The total distance traveled in this test was not different between groups (FIG. 18A), meaning that there was no bias linked to motor deficit or any lack of motivation. Significant deficits in the short-term spatial memory were found 14 days post-surgery in Aβ1-42-injected mice compared to controls (FIG. 18B), demonstrated by the reduced exploration time of the new arm during the test session of the Y-maze. The daily oral administration of 33 (1 mg/kg) was able to prevent the short-term spatial memory deficit in mice injured with Aβ1-42. Injection of oligomers of Aβ1-42 in the CA1 area of the hippocampus induced a significant loss of hippocampal neurons (FIG. 19A), increased the colocalization of neurons with AT100 (FIG. 19B) and associated with a large activation of microglial cells (FIG. 19C). 33 daily administered from day 1 to day 28 at 1 mg/kg significantly improved the survival of NeuN(+) neurons. 33 was also able to decrease the AT100 into the neurons and to significantly decrease the activation of Iba1(+) microglial cells (FIG. 19).

Altogether, the results show beneficial effects of a chronic treatment of 33 on the short-term memory in an animal model of Alzheimer's disease, based on the toxicity of amyloid beta 1-42 oligomers in aged mice. The effects on the short-term memory are associated with protective effects of 33 on hippocampal neurons, reduction of hyperphosphorylated Tau protein and neuroinflammation decrease.

### Example 12: Effects of a Compound in an In Vivo Model of Heart Failure

Generation of a Rat Model of Cardiac Impairment Following Myocardial Infarction

Fifty (50) Wistar male rats from Janvier Labs (France), 6-week-old (200-224 g on the day of arrival), were included in this study. Acclimatization of animals lasted at least 5 days. During the study, animals had free access to food and drinking water ad libitum.

Myocardial infarction (MI) was induced by chronic left anterior descending coronary artery (LAD) ligation performed at DO. Rats were anesthetized with an intraperitoneal (IP) injection of Medetomidine (0.5 mg/kg) and Ketamine (50 mg/kg), intubated and ventilated at 10 ml/kg tidal volume and 70-80 cycles/min. Body temperature was maintained between 36.5° C. and 37.5° C. via a thermoregulated heating pad connected to a rectal probe. A left lateral thoracotomy was carefully performed so as to expose the heart. Then, a suture was placed around the LAD (4/0 Silk, Ethicon), around 2 mm below the left atrium and close to the interventricular junction, in order to obtain infarct size (IS) near 40% of the total left ventricular (LV) area. Chest was closed, air was expelled from the rib cage to avoid pneumothorax and quick reanimation was performed using atipamezole hydrochloride (IM, 0.5 mg/ml, Zoetis). A peri-operative care of the animals was performed during the surgery:
Pre-operative care by one carprofen injection (SC, 5 mg/kg, Pfizer) and one lidocaine injection on the site of incision (SC, 4 mg/kg, Vetoquinol).
Post-operative care: 3 injections of buprenorphine (SC, 0.01 mg/kg, Axience) administered after awakening and during the first 24 hours after the surgery.

Sham animals from were subjected to the same protocol than MI groups. However, after the left lateral thoracotomy exposing the heart, the rib cage was closed without passing the suture thread around LAD.

Echocardiography

Rats underwent transthoracic echocardiographic examination in order to assess cardiac morphology and function in a noninvasively way. Echocardiography was performed by using a digital ultrasound system (Vivid 7, GE Medical Systems) equipped with a 10 MHz phased array and a 13 MHz linear-array transducer. All images acquisitions, measurements and calculations were done in accordance to the American Society of Echocardiography (Schiller et al. 1999) and previous validation of the method in rat (Litwin et al. 1994). Rats were anesthetized with 4% isoflurane in 50% oxygen-50% air mixture and maintained with 2-2.5% isoflurane during the procedure. They were placed in supine position on a heating pad and thorax-shaved. Body temperature was monitored and maintained between 36.5-37.5° C. via a rectal probe connected to a thermoregulated heating pad. Standard B-mode (Brightness-mode) and M-mode (Motion-mode) images of the heart were obtained in the two-dimensional (2D) parasternal long axis view (PSLA). LV parameters were measured and calculated as the mean of 3 consecutive cardiac cycles by a single blinded trained operator. Left ventricle (LV) function was assessed using ejection fraction (EF), calculated as follow: EF=ejection fraction=[(End diastolic volume-End systolic volume)/End diastolic volume]*100. FS=fractional shortening=[(LVIDd-LVIDs)/LVIDd]*100.

Other parameters were measured for animal randomization, and for the calculation of FS: LVIDd and LVIDs=LV internal diameter in diastole and systole.

A total of two (2) echocardiographic exams were performed for all the animals. The first examination took place 5 days after surgery. It was used as a control of the surgery to exclude rats with small infarcts and limited reduction of EF. Animals were randomized in three homogeneous groups based on LVIDd and EF. After the randomization, a longitudinal follow-up study by echocardiography was performed on the 3 groups of rats. The 2nd echocardiographic examinations were done at Month 2 (M2) following the start of the treatments. They were used to assess the cardiac remodeling and function following MI, based on the echocardiographic parameters previously described.

Treatment of the Animals

Rats were administered by oral gavage from day 5 post-surgery up to 2 months following the start of the treatments. Groups were split as follow:
Group 1: Sham surgery, vehicle carboxylethylcellulose (CMC) 0.5%, n=10
Group 2: MI, CMC 0.5%, n=20
Group 3: MI, 66 0.5 mpk, n=20

Statistics

Statistical analysis was performed with the Graphpad 6 software. If values were normally distributed, a parametric analysis was performed. First, differences were assessed between Sham/Vehicle and MI/Vehicle using an unpaired test followed by the appropriate post-hoc test.

Then, the MI/66 group was compared to MI/Vehicle using an unpaired t-test followed by the appropriate post-hoc test.

Results

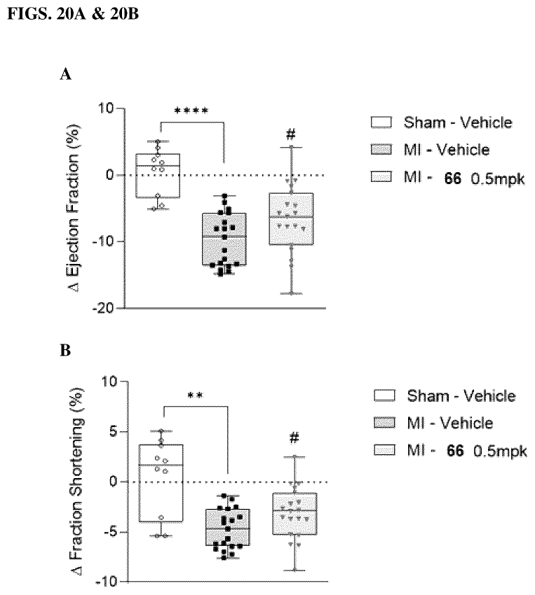

Progression of left ventricle (LV) dysfunction induced by myocardial infarct caused a significant reduction of EF and FS (p<0.0001 & p<0.01, MI/Vehicle vs Sham/Vehicle group; FIG. 20). 66 prevented the LV dysfunction MI-induced, with significant increases of EF and FS parameter in M-mode (p<0.05 and p<0.05, MI/66 vs MI/Vehicle; FIG. 20).

In conclusion, 66 (0.5 mg/kg) was able to prevent LV systolic dysfunction induced by MI 2 months after LAD ligation, with a significant improvement of systolic function (EF & FS).

### Example 13: Testing of Compounds in Primary Myoblasts Derived from Patients Suffering from Sporadic Inclusion Body Myositis

Human primary myoblasts lines are purchased from “Hospices Civils de Lyon.” Sporadic inclusion body myositis myoblasts (sIBM) are derived from a patient. The sIBM diagnosis in the patients is validated by the “Hospices Civils de Lyon” through scores for inflammation, p62-positive aggregates in fibers and vacuoles, mitochondrial dysfunction, and indicative clinical signs. Cells were grown in DMEM/F-10, supplemented with 12% FBS (GIBCO) and penicillin-streptomycin (1 3, GIBCO). Cells are cultured at 37° C. in a 5% CO₂ atmosphere and tested for mycoplasma using Mycoprobe (CULOO1B, R&D systems).

Cells are treated for 24 h with small molecules at indicated concentrations. Protein aggregates are detected using the Proteostat® dye and the Aggresome Detection kit (ENZ-51035-K100) was purchased from Enzo Life Sciences, Inc and all components were prepared according to manufacturer's instruction.

Confocal images are acquired with Zeiss LSM 700 Upright confocal microscope (Carl Zeiss AG) under non-saturating exposure conditions.

Quantitative analysis of the images is carried out by histogram analysis of the fluorescence intensity at each pixel across the images using ImageJ (Fiji; National Institutes of Health). Appropriate thresholding is employed to all the images of each single experiment to eliminate background signal in the images before histogram analysis. Fluorescence intensity and signal positive areas are calculated using the integrated “analyse particles” tool of the Fiji software.

Results show that treatment with small molecules lead to a reduction of protein aggregates in sIBM derived primary myoblasts.

### Example 14: Effects of Test Compounds on Mitochondrial Content in Mouse T Lymphocytes

In Vitro T Cell Isolation and Stimulation

Splenic murine T cells were isolated from mice aged 6-20 weeks by negative selection with EasySep mouse T cell isolation kit (Stem cell) according to the manufacturer's instructions. T cell activation was initiated using CD3/CD28 activation beads (11456D; ThermoFisher Scientific) at a ratio of 25 μl dynabeads per 0.5×10⁶ T-cells. Cells were activated with test compounds at 2 μM or DMSO in T cell activation Medium (RPMI (ThermoFisher Scientific) 10% FBS (South America origin), 10 mM Hepes (Sigma), 1× Non-Essential Amino Acid, 1 mM Sodium Pyruvate, 50 μM β-Mercaptoethanol, 100 U/ml penicillin, 100 μg/ml streptomycin and 2 mM Glutamax (ThermoFisher Scientific) for 72 h. For subsequent analysis, activating beads were magnetically removed prior staining.

Staining

For flow cytometric analysis of mitochondria 500 μl of Mitotracker Red (200 nM; ThermoFisher) was applied followed by surface staining.

Flow Cytometry and Data Acquisition

Cell doublets and cells debris were excluded from the analysis by gating FSC-A vs FSC-H followed by SSC-A vs SSC-H dot plots and FSC-A vs SSC-A dot plots, respectively. eFluor 780 highly positive cells, i.e, dead cells, were gated out of the analysis. Data was acquired on Canto II (BD), Fortessa (BD) and Aurora (Cytek) cytometers and analyzed with FlowJo.

Results

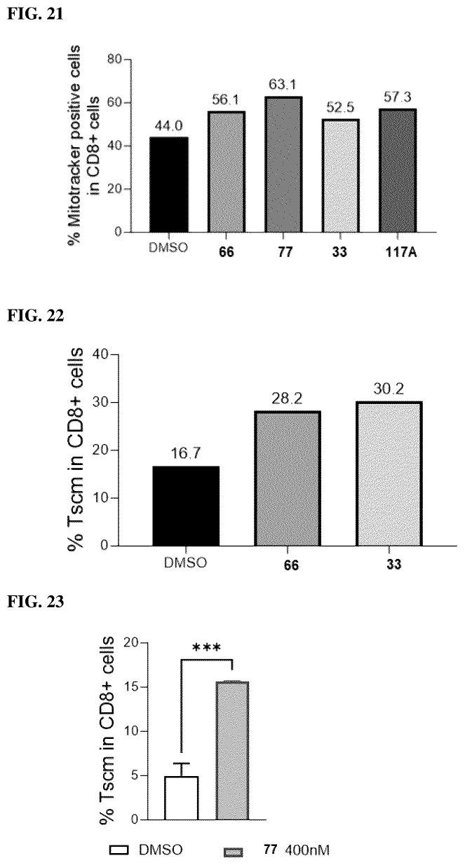

Test compounds were able to stimulate mitochondrial biogenesis, which is known to be triggered subsequently to the induction of mitophagy, in mouse T lymphocytes, as shown in the FIG. 21. In particular, there was an increase in the percentage of Mitotracker positive cells of 27.5% for 66 (56.1% vs 44% for DMSO), +43.4% for 77 (63.1% vs 44% for DMSO), +19% for 33 (52.5% vs 44% for DMSO) and +30% for 117A (57.3% vs 44% for DMSO).

### Example 15: Effects of Test Compounds on the Proportion of Mouse T Memory Stem Cells (Tscm)

T cells were isolated and stimulated as described in Example 14.

Staining

Stimulated T cells were stained with αCD44-AF400 (IM7; 103026; Biolegend), αCD62L BV785 (MEL-14; 104440; Biolegend) and αSca-1-PerCP-Cy5.5 (D7; 45-5981-82; eBioscience).

Tscm represent a subset of minimally differentiated T cells, which share a CD44^{lo}CD62L^{hi} phenotype with naïve T cells, but are phenotypically distinct by expressing high levels of Sca1 (Gattinoni et al., 2009). Tscm are therefore identified as CD44⁻CD62L⁺Sca1^{hi} cells.

Flow Cytometry and Data Acquisition

Flow cytometry and analysis was performed as described in Example 14.

Results

Test compounds were able to increase the percentage of CD44⁻CD62L⁺Sca1^{hi} Tscm cells, as shown in the FIG. 22. In particular, there was an increase in the percentage of Tscm in CD8+ cells of 69% for 66 (28.2% vs 16.7% for DMSO) and +81% for 33 (30.2% vs 16.7% for DMSO).

### Example 16: Effects of Test Compounds on the Proportion of Human T Memory Stem Cells (Tscm)

In Vitro T Cell Isolation and Stimulation

Human peripheral blood mononuclear cells were purified with a Ficoll-Paque (GE healthcare) gradient. T cells were enriched with EasySep Human T Cell Isolation Kit (StemCell) and activated with dynabead Human T-Activator CD3/CD28 for T Cell Expansion and Activation Kit (11131D; ThermoFisher Scientific). Cells were activated with the 77 at 400 nM or DMSO.

Staining

Staining was performed as described in Example 15.

Flow Cytometry and Data Acquisition

Flow cytometry and analysis was performed as described in Example 14.

Results

Results shown in FIG. 23 demonstrate that there is a significant increase (˜3-fold) in Tscm proportion of CD8+ lymphocytes following stimulation with 77. This increase in the percentage of Tscm is known to be associated with an improved response to immunotherapy and can be exploited for CAR-T cell generation.

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES CITED

Abou-El-Enein, M., et al. 2021. Scalable Manufacturing of CAR T cells for Cancer Immunotherapy. Blood cancer discovery 2, 408-422.
Andreux, P. A., et al. 2019. The mitophagy activator urolithin A is safe and induces a molecular signature of improved mitochondrial and cellular health in humans. Nature metabolism, 595-603.
Arcangeli, S., et al. 2022. CAR T-cell manufacturing from naïve/stem memory T-lymphocytes enhances antitumor responses while curtailing cytokine release syndrome. J Clin Invest, 132(12), e150807.
Bernkopf, D. B., et al. 2018. Pgam5 released from damaged mitochondria induces mitochondrial biogenesis via Wnt signaling. The Journal of cell biology 217, 1383-1394.
Boakye, Y. D., Groyer, L., Heiss, E. H., 2018. An increased autophagic flux contributes to the anti-inflammatory potential of urolithin A in macrophages. Biochimica et biophysica acta. General subjects 1862, 1, 61-70.
Buck, M. D., et al. 2016. Mitochondrial Dynamics Controls T Cell Fate through Metabolic Programming. Cell 166, 63-76.
Chen, D. S., Mellman, I., 2017. Elements of cancer immunity and the cancer-immune set point. Nature 541, 321-330.
Cortés-Martin, A. et al. 2018. The gut microbiota urolithin metabotypes revisited: the human metabolism of ellagic acid is mainly determined by aging. Food & function 9, 4100-4106.
D'Amico, D. et al. 2021. Impact of the Natural Compound Urolithin A on Health, Disease, and Aging. Trends in molecular medicine 27, 687-699.
Dumauthioz, N., et al. 2020. Enforced PGC-1α expression promotes CD8 T cell fitness, memory formation and antitumor immunity. Cellular & molecular immunology.
Luca Gattinoni, et al. A human memory T cell subset with stem cell like properties. 2011 Nature medicine, 17, 1290-1297
Espín, J. C., et al. 2013. Biological significance of urolithins, the gut microbial ellagic Acid-derived metabolites: the evidence so far. Evidence-based complementary and alternative medicine: eCAM 2013, 270418.
Finck, B. N., Kelly, D. P., 2006. PGC-1 coactivators: inducible regulators of energy metabolism in health and disease. The Journal of clinical investigation 116, 615-622.
Gamero, A. M., Larner, A. C., 2000. Signaling via the T cell antigen receptor induces phosphorylation of Statl on serine 727. The Journal of biological chemistry 275, 16574-16578.
Gattinoni, L., et al. 2017. T memory stem cells in health and disease. Nature medicine 23, 18-27.
Gattinoni, L., et al. 2009. Wnt signaling arrests effector T cell differentiation and generates CD8+ memory stem cells. Nature medicine 15, 808-813.
Ghosh, S. et al. 2021. Microbial metabolite Urolithin A induces expansion of Regulatory T Cells in aryl hydrocarbon receptor (AhR)-dependent manner. The Journal of Immunology 206, 113.03.
Hombach, A., et al. 2001. Tumor-specific T cell activation by recombinant immunoreceptors: CD3 zeta signaling and CD28 costimulation are simultaneously required for efficient IL-2 secretion and can be integrated into one combined CD28/CD3 zeta signaling receptor molecule. Journal of immunology (Baltimore, Md.: 1950) 167, 6123-6131.
Ichii, H., et al. 2002. Role for Bcl-6 in the generation and maintenance of memory CD8+ T cells. Nature immunology 3, 558-563.
Jaganathan, S. K., et al. 2014. Role of pomegranate and citrus fruit juices in colon cancer prevention. World journal of gastroenterology 20, 4618-4625.
June, C. H. et al. 2018. CAR T cell immunotherapy for human cancer. Science (New York, N.Y.) 359, 1361-1365.
Kratchmarov, R., Magun, A. M., Reiner, S. L., 2018. TCF1 expression marks self-renewing human CD8+ T cells. Blood advances 2, 1685-1690.
Litwin, S. E. et al. 1999. Serial echocardiographic assessment of left ventricular geometry and function after large myocardial infarction in the rat. Circulation, 89, 11, 345-354.
Le, D. T. et al. 2015. PD-1 Blockade in Tumors with Mismatch-Repair Deficiency. The New England Journal of medicine 372, 2509-2520.
Luan, P., et al. 2021. Urolithin A improves muscle function by inducing mitophagy in muscular dystrophy. Science translational medicine 13.
Gattinoni, L. et al. 2012. Paths to stemness: building the ultimate antitumour T cell. Nature Reviews, 12, 671-684.
Majzner, R. G., Mackall, C. L., 2019. Clinical lessons learned from the first leg of the CAR T cell journey. Nature medicine 25, 1341-1355.
Mo, F., et al. 2021. An engineered IL-2 partial agonist promotes CD8+ T cell stemness. Nature 597, 544-548.
Nicolas, A. M., et al. 2022. Inflammatory fibroblasts mediate resistance to neoadjuvant therapy in rectal cancer. Cancer cell 40, 168-184.e13.
Pagès, F., et al. 2018. International validation of the consensus Immunoscore for the classification of colon cancer: a prognostic and accuracy study. The Lancet 391, 2128-2139.
Parisi, G., et al. 2020. Persistence of adoptively transferred T cells with a kinetically engineered IL-2 receptor agonist. Nature communications 11, 660.
Pfeiffer, A., et al. 2018. In vivo generation of human CD19-CAR T cells results in B-cell depletion and signs of cytokine release syndrome. EMBO molecular medicine 10.
Pilipow, K., et al. 2018. Antioxidant metabolism regulates CD8+T memory stem cell formation and antitumor immunity. JCI insight 3.
Reschke, R., et al. 2021. Immune cell and tumor cell-derived CXCL10 is indicative of immunotherapy response in metastatic melanoma. Journal for immunotherapy of cancer 9, e003521.
Roberto, A., et al. 2015. Role of naive-derived T memory stem cells in T-cell reconstitution following allogeneic transplantation. Blood 125, 2855-2864.
Rosenberg, S. A., Restifo, N. P., 2015. Adoptive cell transfer as personalized immunotherapy for human cancer. Science (New York, N.Y.) 348, 62-68.
Roychoudhuri, R., et al. 2016. BACH2 regulates CD8(+) T cell differentiation by controlling access of AP-1 factors to enhancers. Nature immunology 17, 851-860.
Ryu, D., et al. 2016. Urolithin A induces mitophagy and prolongs lifespan in C. elegans and increases muscle function in rodents. Nature medicine, 879-888.
Sato, T., et al. 2011. Long-term expansion of epithelial organoids from human colon, adenoma, adenocarcinoma, and Barrett's epithelium. Gastroenterology 141, 1762-1772.
Scharping, N. E., et al. 2016. The Tumor Microenvironment Represses T Cell Mitochondrial Biogenesis to Drive Intratumoral T Cell Metabolic Insufficiency and Dysfunction. Immunity 45, 374-388.
Schillert et al. 1989. Recommendations for quantitation of the left ventricle by two-dimensional echocardiography. American Society of Echocardiography Committee on Standards, Subcommittee on Quantitation of Two-Dimensional Echocardiograms. J Am Soc Echocardiogr 2(5), 358-67.
Schmitt, M., Greten, F. R., 2021. The inflammatory pathogenesis of colorectal cancer. Nature reviews. Immunology 21, 653-667.
Schnalzger, T. E., et al. 2016. Modulation of mTOR Signalling Triggers the Formation of Stem Cell-like Memory T Cells. EBioMedicine 4, 50-61.
Schumann, J., Stanko, K., Schliesser, U., Appelt, C., Sawitzki, B., 2015. Differences in CD44 Surface Expression Levels and Function Discriminates IL-17 and IFN-7 Producing Helper T Cells. PloS one 10, e0132479.
Schwank, G., Clevers, H., 2016. CRISPR/Cas9-Mediated Genome Editing of Mouse Small Intestinal Organoids. Methods in molecular biology (Clifton, N.J.) 1422, 3-11.
Seeram, N. P., et al. 2005. In vitro antiproliferative, apoptotic and antioxidant activities of punicalagin, ellagic acid and a total pomegranate tannin extract are enhanced in combination with other polyphenols as found in pomegranate juice. The Journal of nutritional biochemistry 16, 360-367.
Shen, P.-X., et al. 2021. Urolithin A ameliorates experimental autoimmune encephalomyelitis by targeting aryl hydrocarbon receptor. EBioMedicine 64, 103227.
Sung, H., Ferlay, J., Siegel, R. L., Laversanne, M., Soerjomataram, I., Jemal, A., Bray, F., 2021. Global Cancer Statistics 2020: GLOBOCAN Estimates of Incidence and Mortality Worldwide for 36 Cancers in 185 Countries. CA: A cancer journal for clinicians 71, 209-249.
Toney, A. M., Fox, D., Chaidez, V., Ramer-Tait, A. E., Chung, S., 2021. Immunomodulatory Role of Urolithin A on Metabolic Diseases. Biomedicines 9.
Totiger, T. M., et al. 2019. Urolithin A, a Novel Natural Compound to Target PI3K/AKT/mTOR Pathway in Pancreatic Cancer. Molecular cancer therapeutics, 301-311.
Utzschneider, D. T., et al. 2016. T Cell Factor 1-Expressing Memory-like CD8(+) T Cells Sustain the Immune Response to Chronic Viral Infections. Immunity 45, 415-427.
van de Wetering, M., et al. 2015. Prospective derivation of a living organoid biobank of colorectal cancer patients. Cell 161, 933-945.
van den Eynde, M., et al. 2018. The Link between the Multiverse of Immune Microenvironments in Metastases and the Survival of Colorectal Cancer Patients. Cancer cell 34, 1012-1026.e3.
Vannini, N., et al., 2019. The NADBooster Nicotinamide Riboside Potently Stimulates Hematopoiesis through Increased Mitochondrial Clearance. Cell stem cell 24, 405-418.e7.
Varga, J., et al. 2020. AKT-dependent NOTCH3 activation drives tumor progression in a model of mesenchymal colorectal cancer. The Journal of experimental medicine 217.
Verma, V., et al. 2021. MEK inhibition reprograms CD8+T lymphocytes into memory stem cells with potent antitumor effects. Nature immunology 22, 53-66.
Weidner, T., et al. 2021. Genetic in vivo engineering of human T lymphocytes in mouse models. Nature protocols 16, 3210-3240.
Xiong, Y., et al. 2020. Inhibiting the coregulator CoREST impairs Foxp3+ Treg function and promotes antitumor immunity. The Journal of clinical investigation 130, 1830-1842.
Yamaguchi, A., et al. 2019. Cleaved PGAM5 is released from mitochondria depending on proteasome-mediated rupture of the outer mitochondrial membrane during mitophagy. Journal of biochemistry 165, 19-25.
Yu, B., et al. 2020a. Mitochondrial phosphatase PGAM5 modulates cellular senescence by regulating mitochondrial dynamics. Nature communications 11, 2549.
Yu, Y.-R., et al. 2020b. Disturbed mitochondrial dynamics in CD8+TILs reinforce T cell exhaustion. Nature immunology 21, 1540-1551.
Zhou, X., Yu, S., Zhao, D.-M., Harty, J. T., Badovinac, V. P., Xue, H.-H., 2010. Differentiation and persistence of memory CD8(+) T cells depend on T cell factor 1. Immunity 33, 229-240.
Ziegler, P. K., et al. 2018. Mitophagy in Intestinal Epithelial Cells Triggers Adaptive Immunity during Tumorigenesis. Cell 174, 88-101.e16.

1. A method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (Ia), 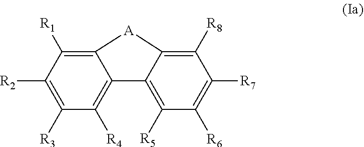
wherein
A is 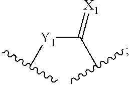
X₁ is selected from O and S;
Y₁ is O;
R₁, R₄, R₅ and R₈ are independently selected from H and halogen;
R₃ and R₆ are independently selected from H, CN, OH, CF₃, halogen, and alkyl;
one of R₂ and R₇ is H, OH, or OAc and the other of R₂ and R₇ is halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, C(O)-alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

2. The compound of claim 1, wherein A is

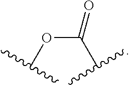 [SMILES: *OC(*)=O]

3. (canceled)

4. The method of claim 1, wherein R₂ is OH.

5.-9. (canceled)

10. The method of claim 1, wherein
R₇ is selected from haloalkyl, substituted cycloalkyl, alkynyl-R₉, OR₁₀, and C(O)NR₁₁R₁₂;
R₉ is selected from OH, substituted cycloalkyl and heterocycloalkyl;
R₁₀ is selected from alkyl, substituted cycloalkyl, heterocycloalkyl and alkyl heterocycloalkyl; and
R₁₁ is H and R₁₂ is alkyl-heterocycloalkyl.

11. The method of claim 10, wherein each occurrence of substituted cycloalkyl is independently substituted with OH, halogen, or hydroxyalkyl.

12. The method of claim 1, wherein R₁, R₃, R₄, R₅, R₆, and R₈ are each H.

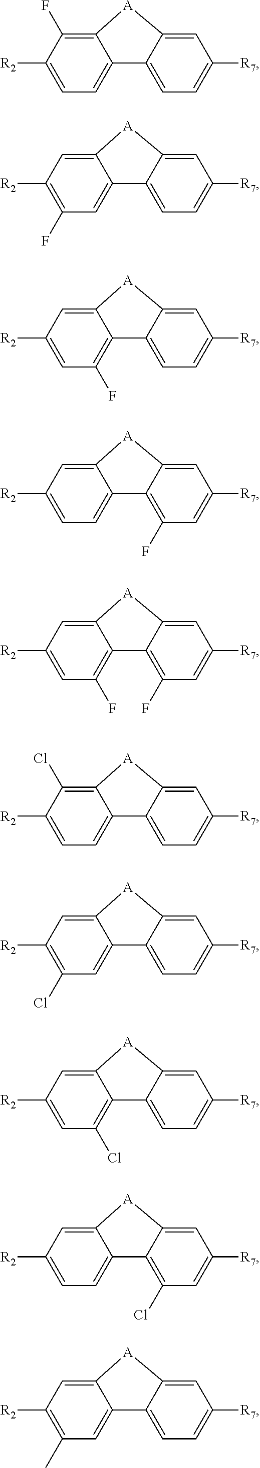

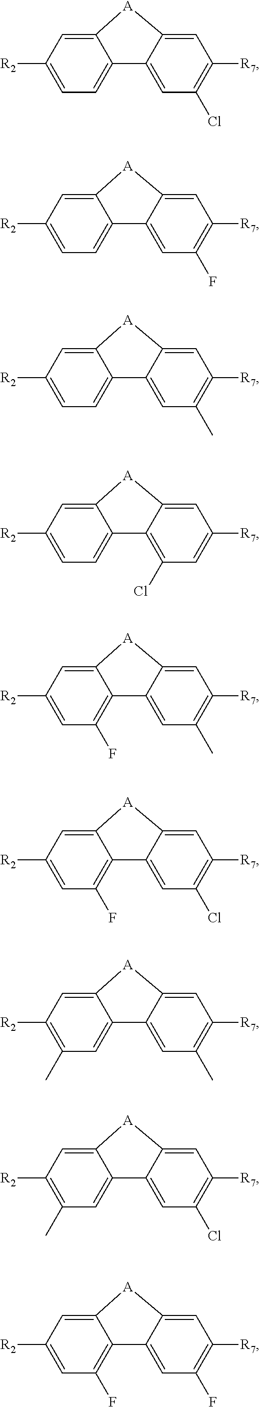

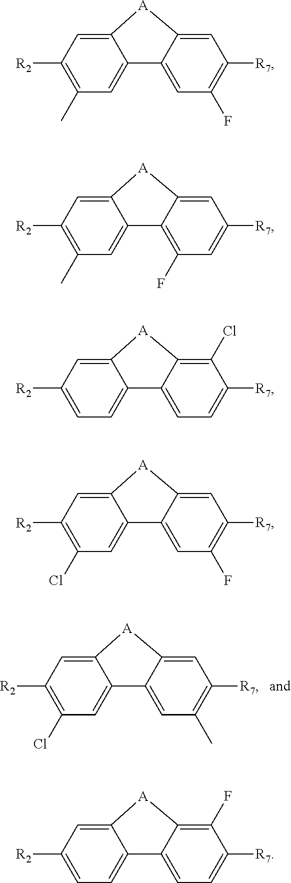

18. The method of claim 1, wherein the compound is selected from:

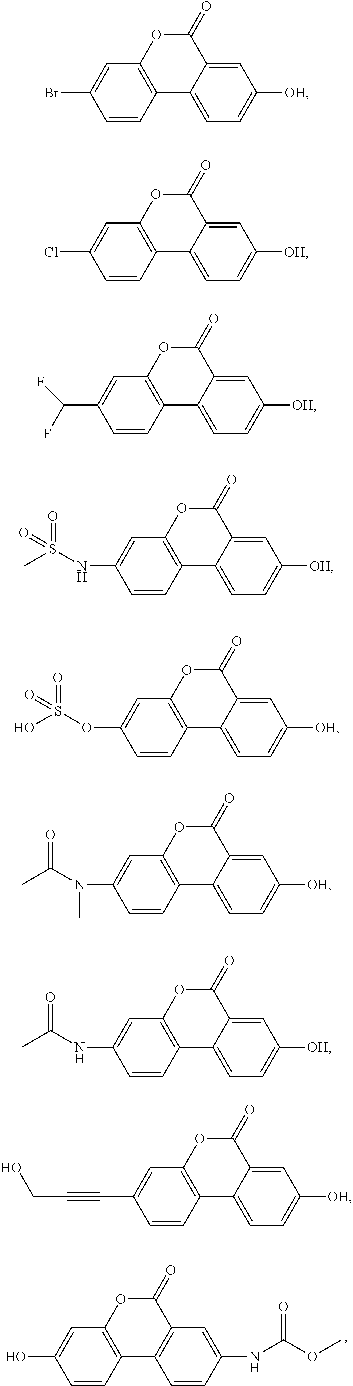

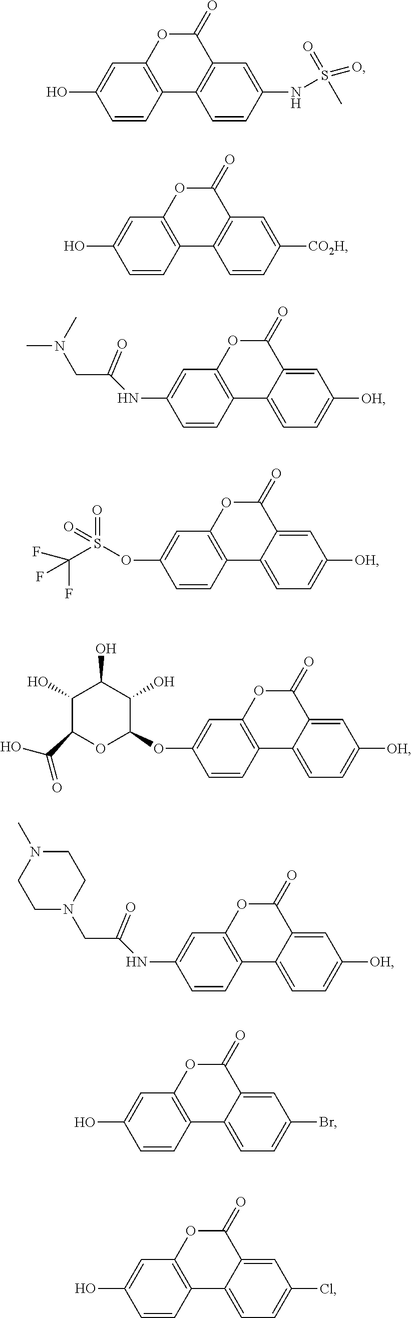

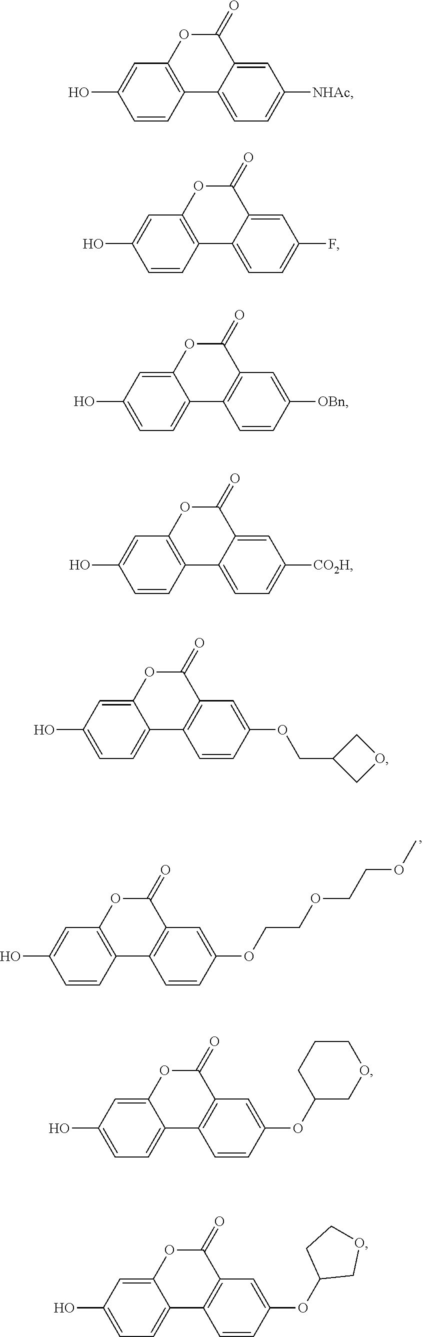

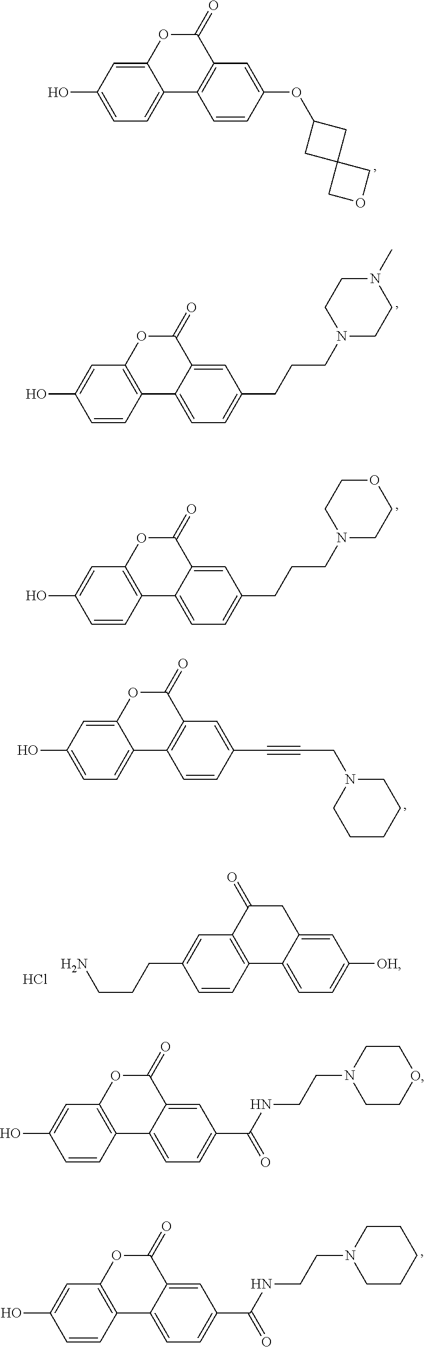

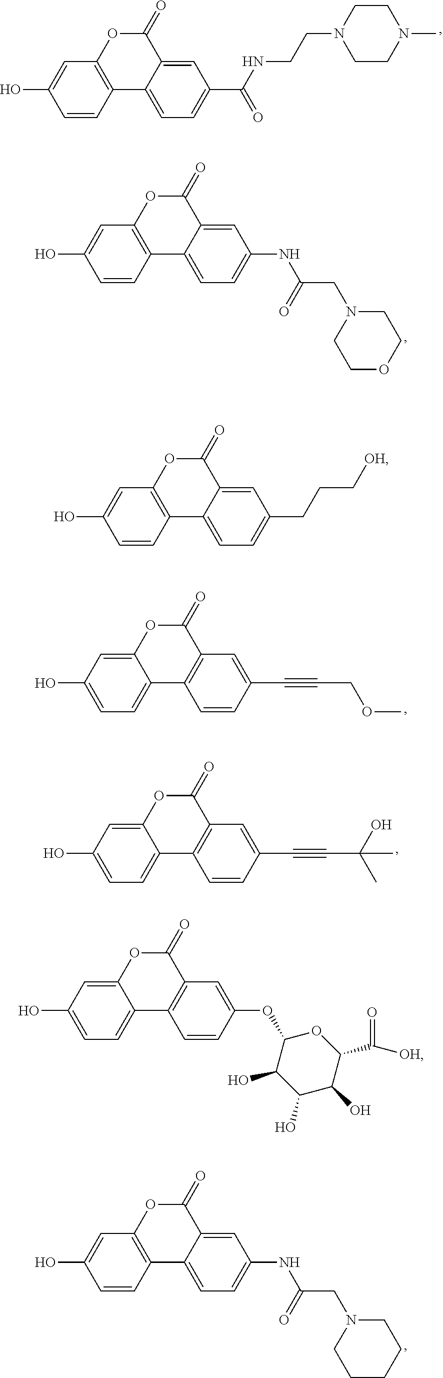

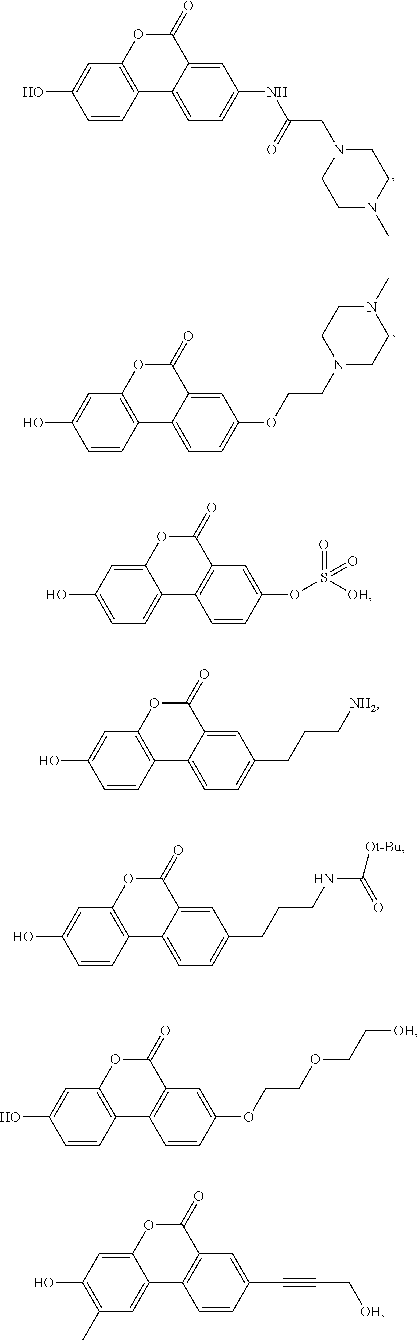

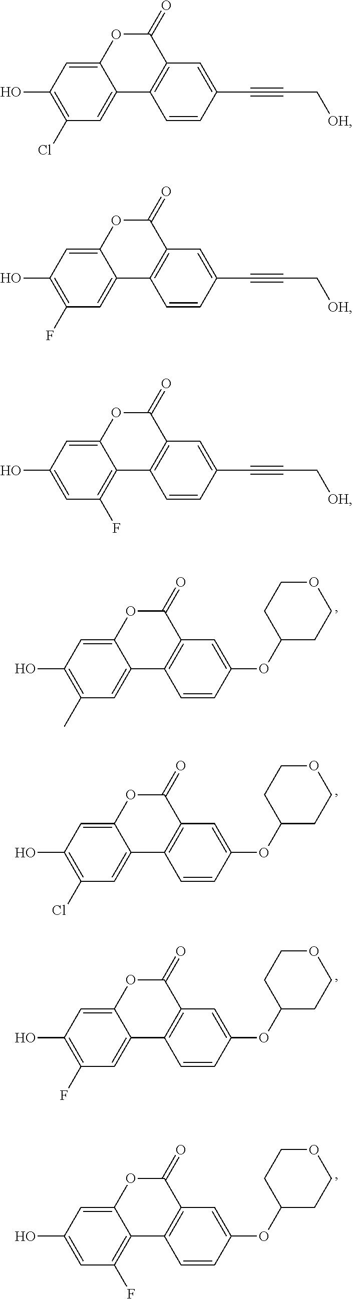

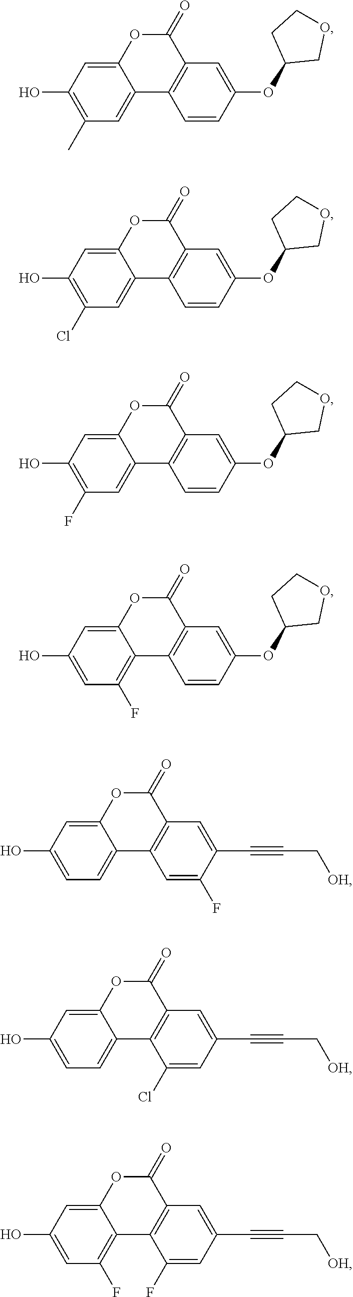

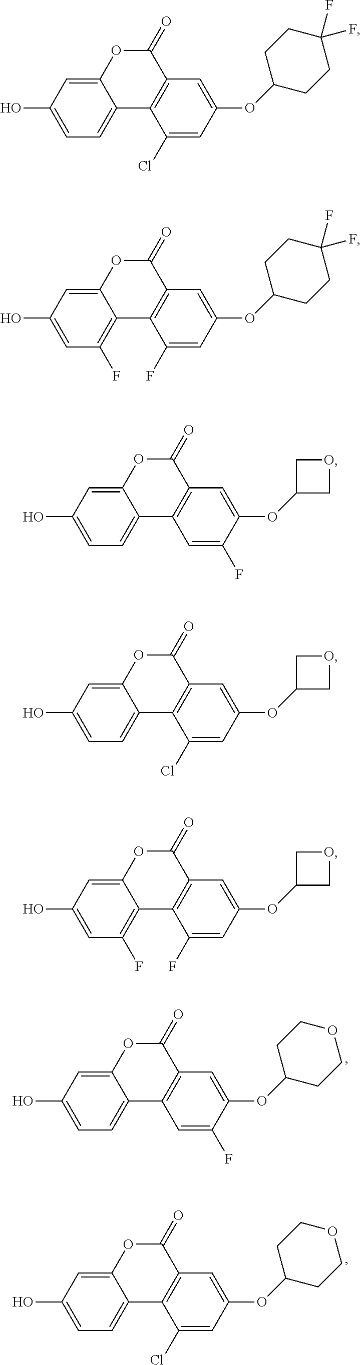

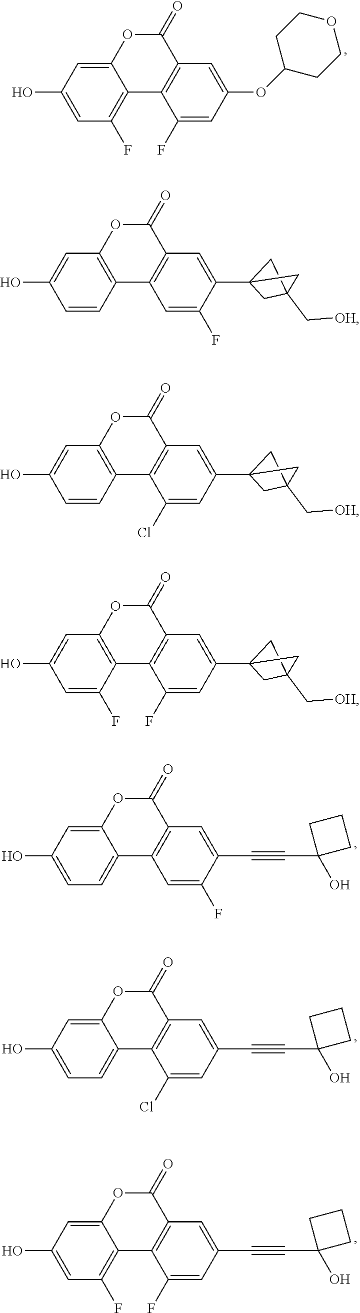

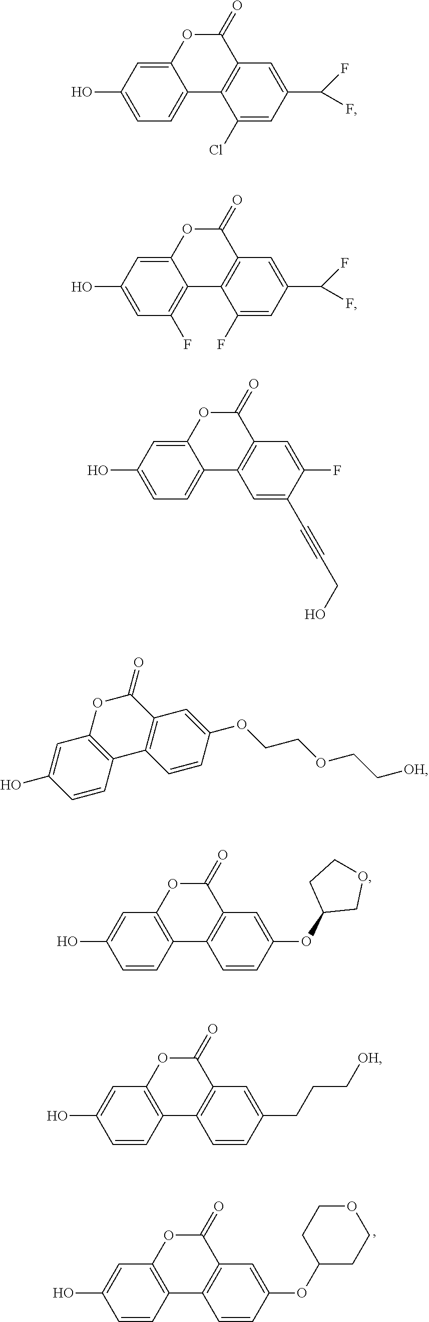

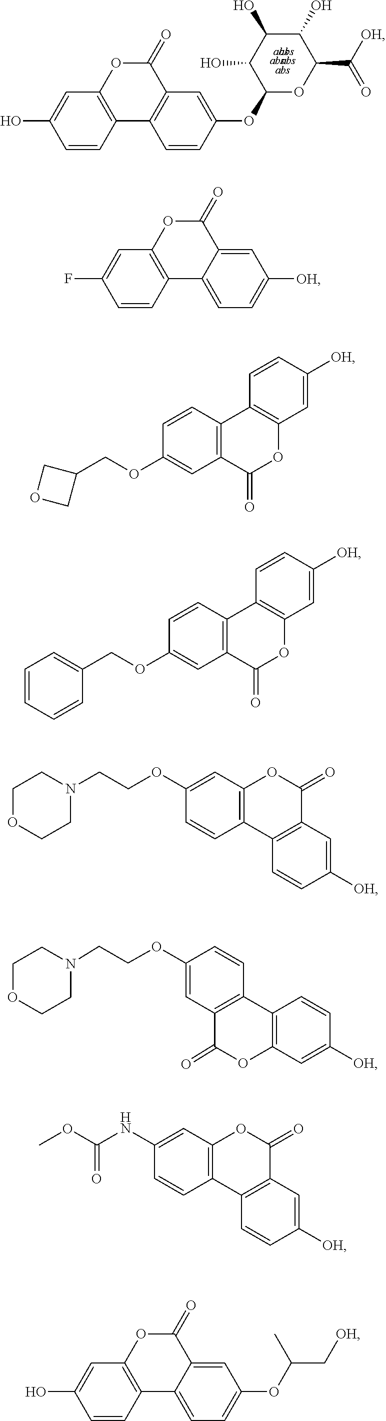

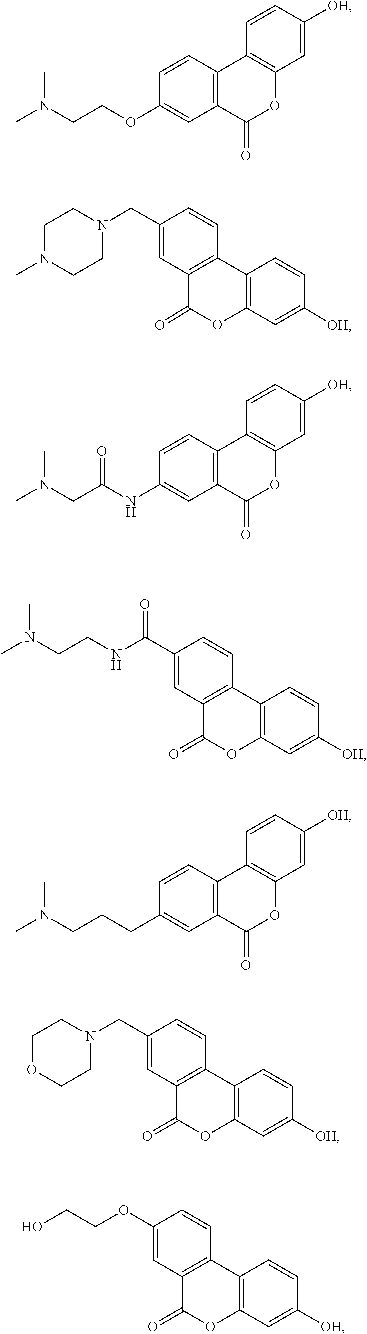

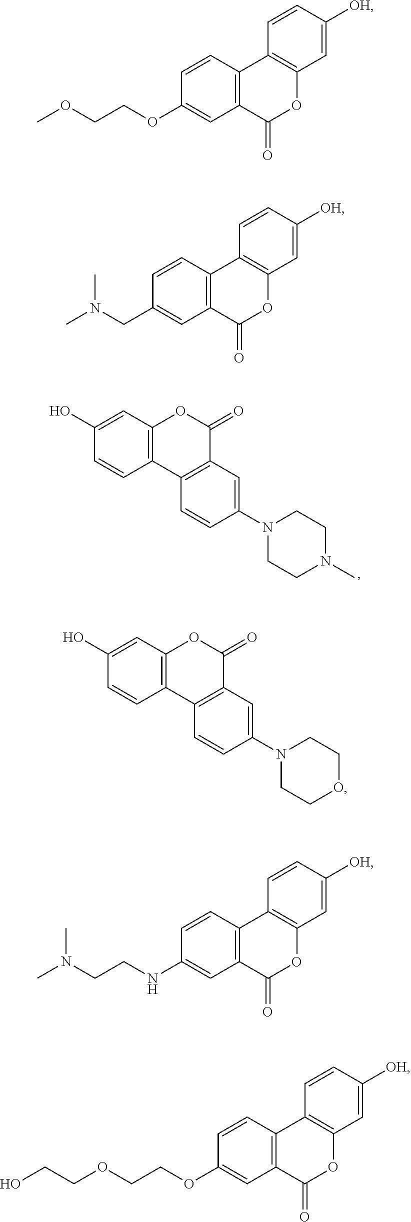

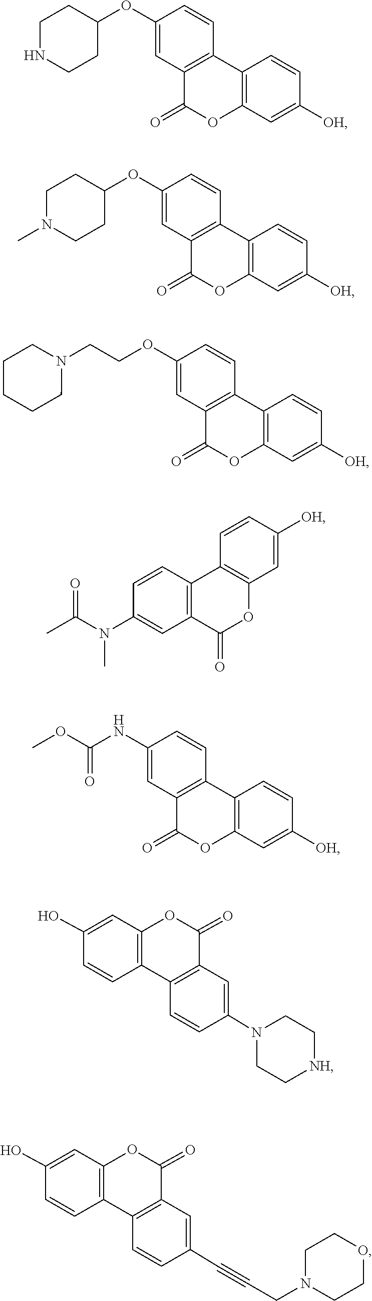

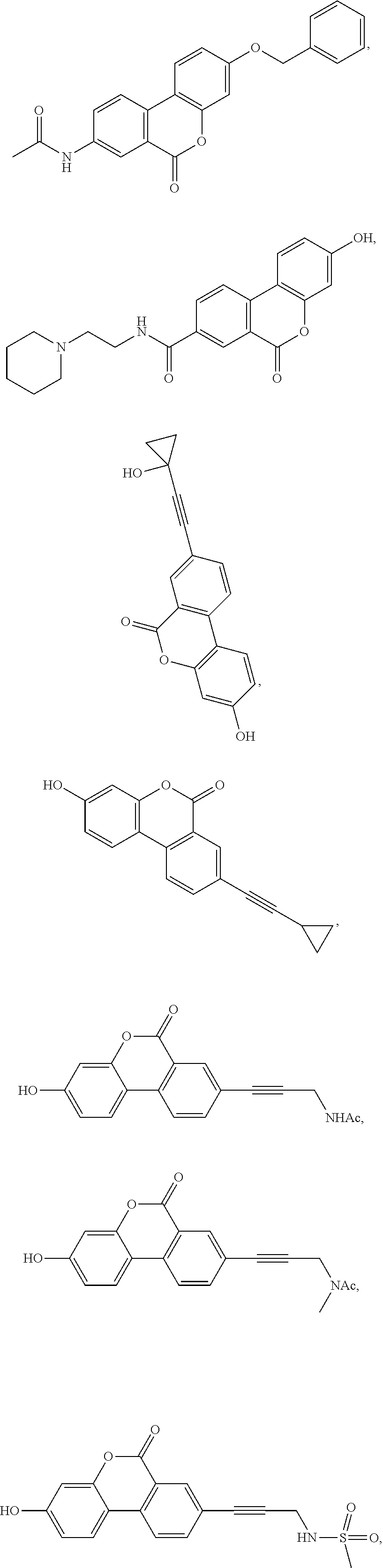

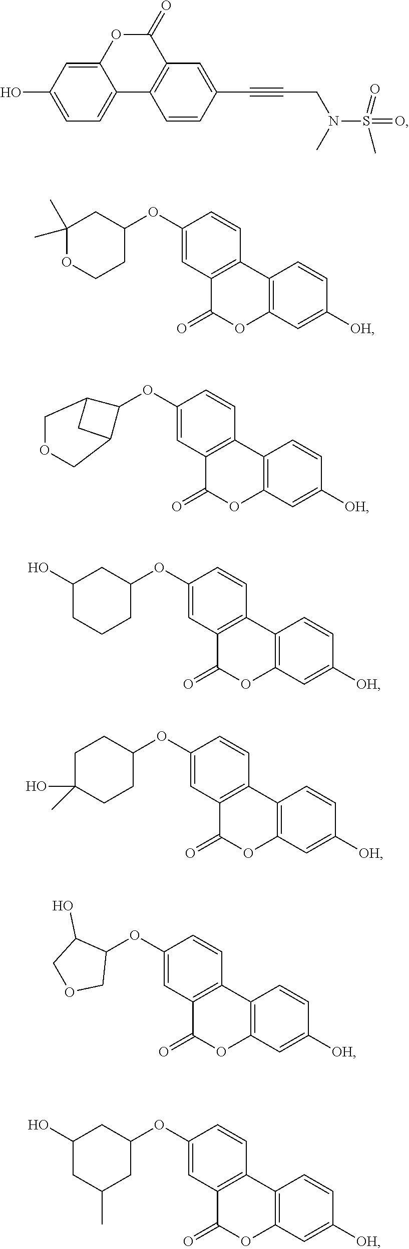

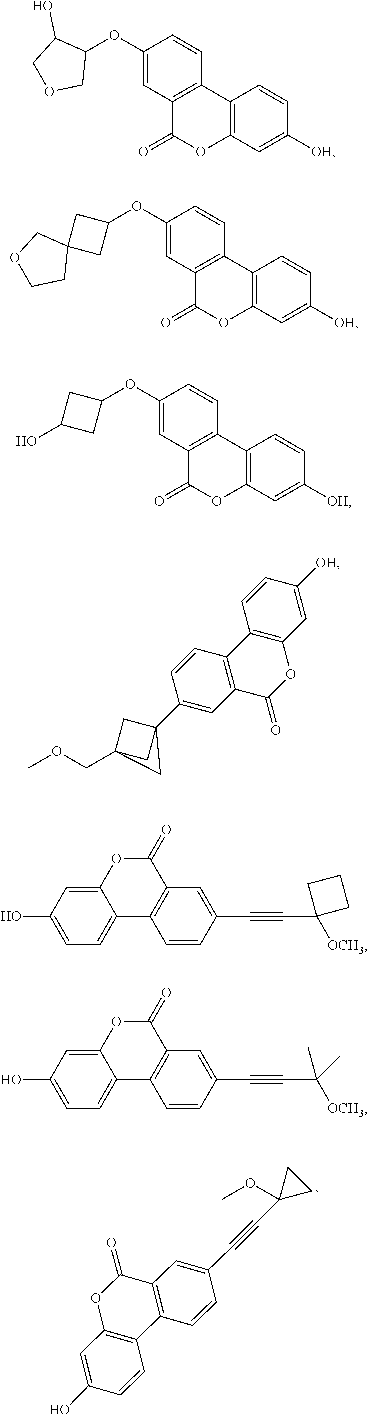

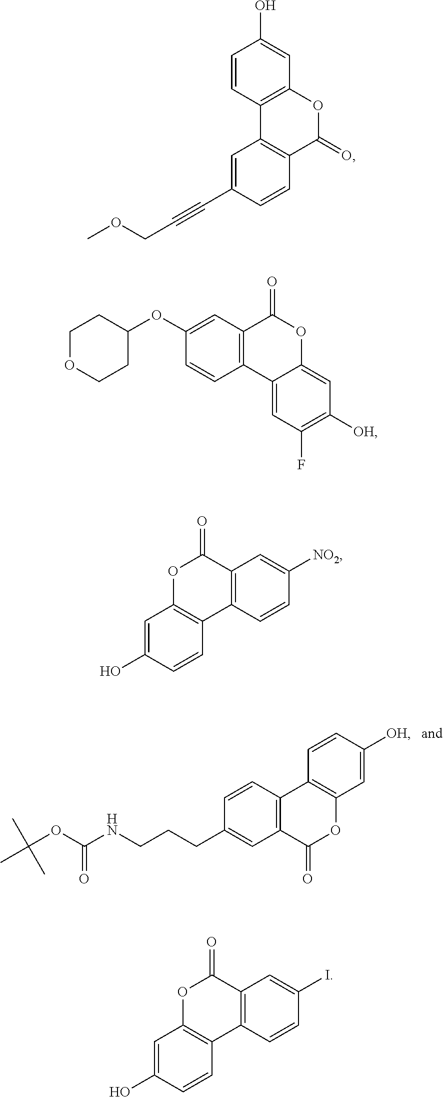 [SMILES: COCC#Cc1ccc2c(=O)oc3cc(O)ccc3c2c1 ; O=c1oc2cc(O)c(F)cc2c2ccc(OC3CCOCC3)cc12 ; O=c1oc2cc(O)ccc2c2ccc([N+](=O)[O-])cc12 ; CC(C)(C)OC(=O)NCCCc1ccc2c(c1)c(=O)oc1cc(O)ccc12 ; "and" ; O=c1oc2cc(O)ccc2c2ccc(I)cc12]

19. The method of claim 1, wherein the compound is selected from:

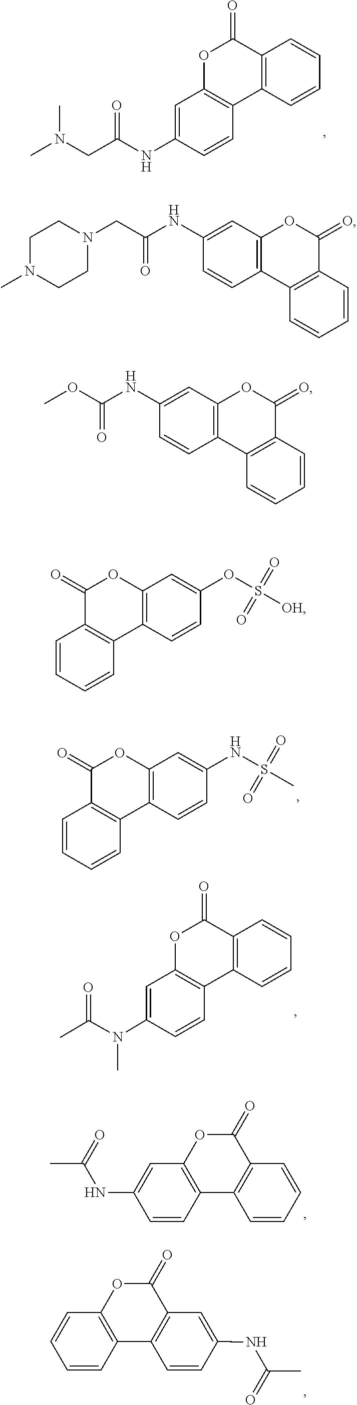

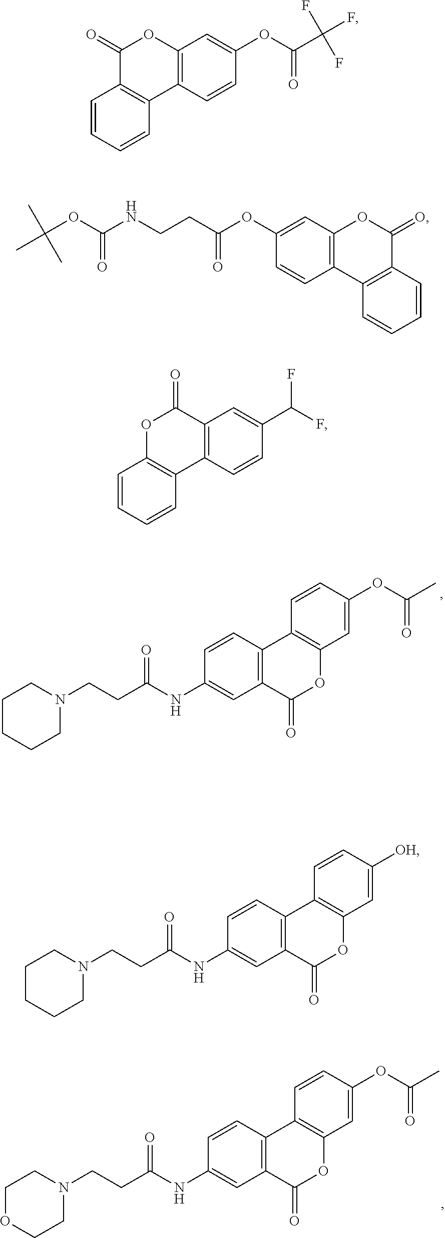 [SMILES: O=C(Oc1ccc2c(c1)oc(=O)c1ccccc12)C(F)(F)F ; CC(C)(C)OC(=O)NCCC(=O)Oc1ccc2c(c1)oc(=O)c1ccccc12 ; O=c1oc2ccccc2c2ccc(C(F)F)cc12 ; CC(=O)Oc1ccc2c(c1)oc(=O)c1cc(NC(=O)CCN3CCCCC3)ccc12 ; O=C(CCN1CCCCC1)Nc1ccc2c(c1)c(=O)oc1cc(O)ccc12 ; CC(=O)Oc1ccc2c(c1)oc(=O)c1cc(NC(=O)CCN3CCOCC3)ccc12]

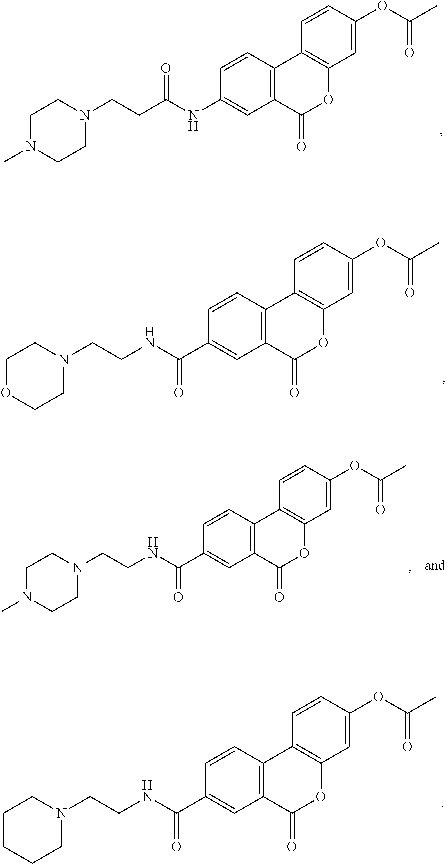

20. (canceled)

21. The method of claim 1, wherein the compound is selected from:

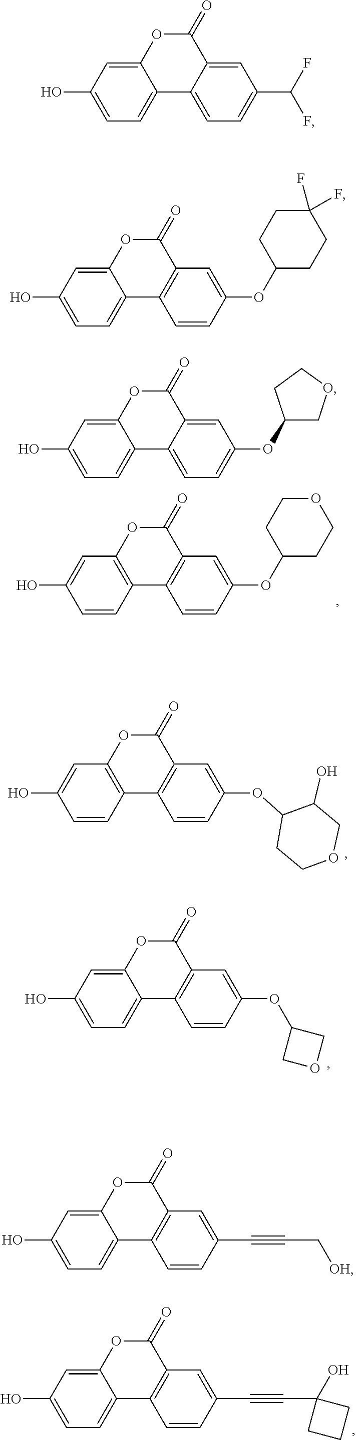

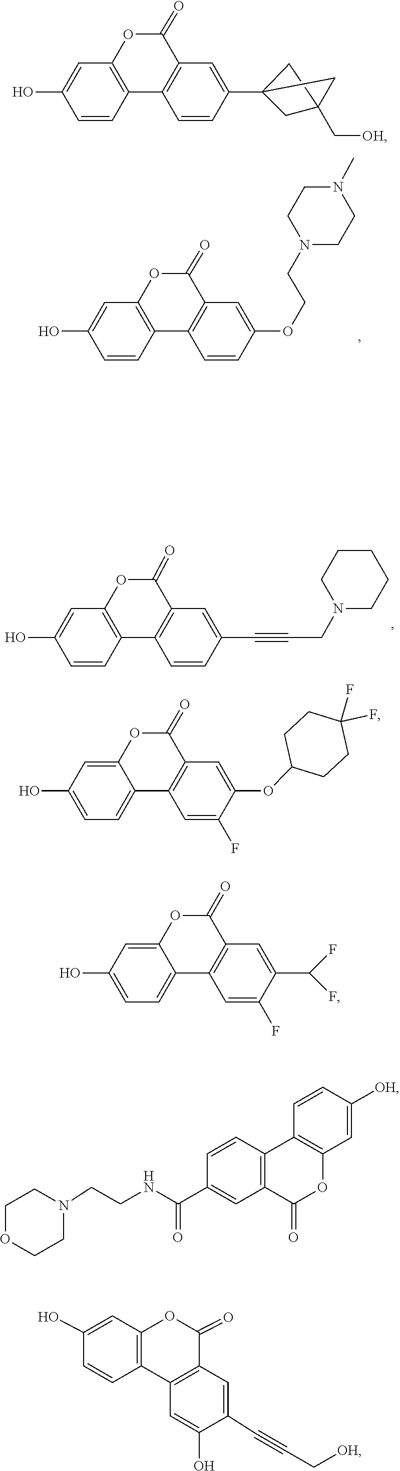

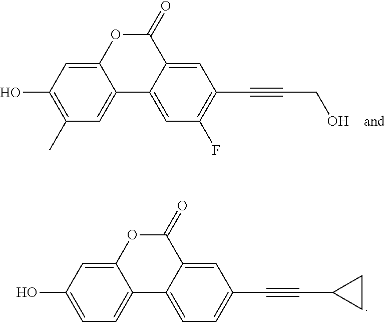 [SMILES: Cc1cc2c(cc1O)oc(=O)c1cc(C#CCO)c(F)cc12 ; O=c1oc2cc(O)ccc2c2ccc(C#CC3CC3)cc12]

22. A method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (Ic),
wherein 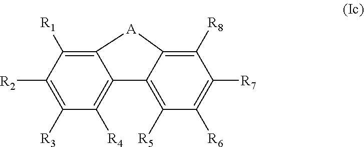
A is 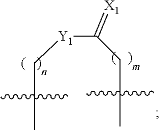
one of n and m is 0; and the other of n and m is 1;
X₁ and Y₁ are each O;
R₁, R₂, R₃, R₆, R₇, and R₈ are independently selected from H, OH, OCH₃, OAc, NH₂, halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
R₄ and R₅ are independently selected from H, halogen and alkyl;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl,
or a pharmaceutically acceptable salt thereof.

23.-35. (canceled)

36. A method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (Id), 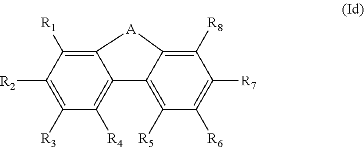
wherein
A is 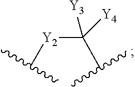
Y₂ is O;
Y₃ and Y₄ are independently selected from H, halogen and alkyl; or together with the carbon to which they are bonded combine to form a cycloalkyl or heterocycloalkyl;
R₁, R₄, R₅, and R₈ are independently selected from H and halogen;
R₂, R₃, R₆, and R₇ are independently selected from H, OH, OCH₃, OAc, NH₂, halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

37.-44. (canceled)

45. A method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (Ie), 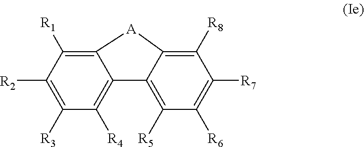
wherein
A is 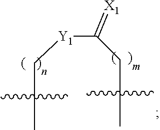
n and m are both 0; or one of n and m is 0, and the other of n and m is 1;
X₁ is O;
Y₁ is selected from NH, N—CH₃, N-t-Bu, N-cycloalkyl, and N-heterocycloalkyl;
R₁, R₂, R₃, R₆, R₇, and R₈ are independently selected from H, OH, OCH₃, OAc, NH₂, halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
R₄ and R₅ are independently selected from H, alkyl, and halogen;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of Ru is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

46.-62. (canceled)

63. A method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (If), 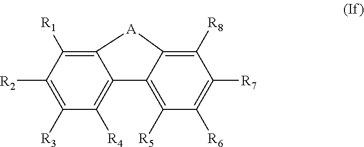
wherein
A is selected from 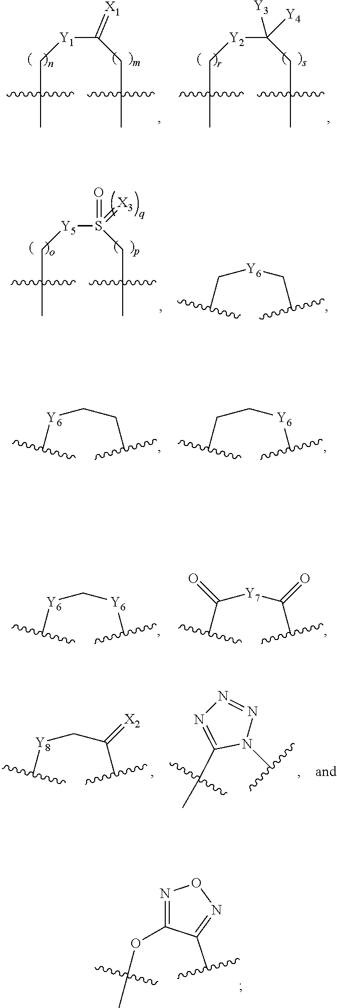
n and m are both 0; or one of n and m is 0 and the other of n and m is 1;
and p are both 0; or one of o and p is 0 and the other of o and p is 1;
q is 0 or 1;
r and s are both 0; or one of r and s is 0 and the other of r and s is 1;
X₁ and X₂ are each O;
X₃ is O or N(alkyl);
Y₁ is S;
Y₂ is selected from O, CH₂, NH, N-alkyl, S, S(O), and SO₂;
Y₃ and Y₄ are independently selected from H, halogen, OH, and alkyl, or together with the carbon to which they are bonded combine to form a cycloalkyl or cycloheteroalkyl;
Y₅ is selected from CH₂, NH, N-alkyl, N-arylalkyl, N-cycloalkyl, and N-heterocycloalkyl;
Each occurrence of Y₆ is independently selected from O, S, S(O), SO₂, NH, N-alkyl, N-alkylaryl, and N-cycloalkyl;
Y₇ is selected from O, NH and N-alkyl;
Y₈ is selected from O and S;
R₁, R₂, R₃, R₆, R₇, and R₈ are independently selected from H, OH, OCH₃, OAc, NH₂, halogen, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂,
R₄ and R₅ are independently selected from H, alkyl, and halogen;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

64.-83. (canceled)

84. A method of treating a neuromuscular disorder, muscle disorder, heart disease, pulmonary fibrosis, liver disease, inflammatory bowel disease, cancer, or cognitive impairment, comprising administering to a subject in need thereof an effective amount of a compound of Formula (Ih), 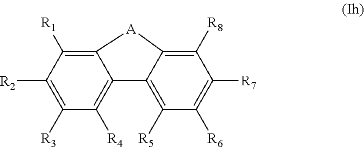
wherein
A is selected from 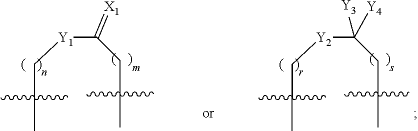
n and m are both 0; or one of n and m is 0 and the other of n and m is 1;
r and s are both 0; or one of r and s is 0 and the other of r and s is 1;
X₁ is O;
Y₁ is selected from O, NH, N-alkyl, and N-cycloalkyl;
Y₂ is O;
Y₃ and Y₄ are independently selected from H, halogen, and alkyl, or together with the carbon to which they are bonded combine to form a cycloalkyl or cycloheteroalkyl;
R₁, R₄, R₅ and R₈ are independently selected from H and halogen;
R₃ and R₆ are independently selected from H, CN, OH, CF₃, halogen, and alkyl;
one of R₂ and R₇ is selected NH₂, NHCH₃, and N(CH₃)₂ and the other of R₂ and R₇ is selected from H, halogen, OCH₃, CN, CF₃, CO₂H, NO₂, NHAc, alkyl, haloalkyl, alkenyl, alkynyl, cycloalkyl, heterocycloalkyl, alkylamino, alkyl-R₉, alkenyl-R₉, alkynyl-R₉, OR₁₀, NHR₁₀, NR₁₁C(O)R₁₂, C(O)NR₁₁R₁₂, and NR₁₁SO₂R₁₂;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from H, C₂-C₁₂ alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of Ru is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
or a pharmaceutically acceptable salt thereof.

85.-108. (canceled)

109. The method of claim 1, wherein the neuromuscular disorder is Charcot-Marie-Tooth disease; the muscle disorder is hereditary inclusion body myositis, oculopharyngeal muscular dystrophy, inclusion body myopathy, Paget's disease of bone, frontotemporal dementia, or Duchenne muscular disorder; the heart disease is heart failure; the pulmonary fibrosis is idiopathic pulmonary fibrosis; the liver disease is non-alcoholic steatohepatitis; the inflammatory bowel disease is ulcerative colitis or Crohn's disease; and the cancer is bladder cancer, breast cancer, cervical cancer, colorectal cancer, esophageal cancer, head and neck cancer, kidney cancer, leukemia, liver cancer, lung cancer, lymphoma, melanoma, prostate cancer, or skin cancer.

110.-121. (canceled)

122. The method of claim 1, wherein the compound enhances the effectiveness of the cancer immunotherapy.

123.-130. (canceled)

131. The method of claim 122, wherein the subject is concurrently being treated with an immune checkpoint inhibitor.

132.-138. (canceled)
